SEQUENCE LISTING (16 sequences)

SEQ ID NO: 1 - DNA, 601 bp, Homo sapiens
  misc_feature 10: n = A,T,C or G
cccccctcgngagtacttctagaattaattaacgcggggtccattctgcgcagtatctcaactgccgttcaacaatcgcaagaggaaggtggagcaggtttcttcatcttacagttgagaaaacagagactcagaagggcttcttagttcatgtttcccttagcgcctcagtgattttttcatggtggcttaggccaaaagaaatatctaaccattcaatttataaataattaggtccccaacgaattaaatattatgtcctaccaacttattagctgcttgaaaaatataatacacataaataaaaaaatatatttttcatttctatttcattgttaatcacaactacttactaaggagatgtatgcacctattggacactgtgcaacttctcacctggaatgagattggacactgctgccctcattttctgctccatgttggtgtccatatagtacttgattttttatcagatggcctggaaaacccagtctcacaaaaatatgaaattatcagaaggattatagtgcaatcttatgttgaaagaatgaactacctcactagtagttcacgtgatgtctgacagatgttgagtttcatt

SEQ ID NO: 2 - DNA, 602 bp, Homo sapiens
  misc_feature 24: n = A,T,C or G
gccccccctcggaagtcttctagnattaattaacgcggggggaaagccttcagtcctttctgtttctttcaactacgtgaaaggattcacagtggagaaagaccctgtaagataattggctttaaattacgagagacttgtgataggacagtaaaacctagagttggagttggatctctggattgtgttatgtcagtgttggtaggttaggaactagatttcccagaatccattccatttgtgattccatgatacaattcaccagtaacctatcttacatgagattcggaagtaagttaagaaggcattagtcatggtttggaagcaccatacagggagacagctgtgtgaatacaggctgtatggacacttgcttccatcccattttcctgcttctttgggttgccaatcaagagtatcctcaaaacgacttgactttaattttctcggaggtgataggcttccacacaggtctccagaagccctgcattgaatatccatccacactttggttttccttcagacattattatgtctgtactaggcaactaattcagactgtcctggttgggaatattctgtgatgctctgactcccctagt

SEQ ID NO: 3 - DNA, 599 bp, Homo sapiens
cgggccccccctcggaagtcttctagaattaattaacgcggggggagcgggcgagcggagttagcagggctttactgcagagcgcgccgggcactccagcgaccgtggggatcagcgtaggtgagctgtggccttttgcgaggtgctgcagccatagctacgtgcgttcgctacgaggattgagcgtctccacccagtaagtgggcaagaggcggcaggaagtgggtacgcaggggcgcaaggcgcacagcctctagacgactcgctttccctccggccaacctctgaagccgcgtcctactttgacagctgcagggccgcggcctggtcttctgtgcttcaccatctacataatgaatcccagtatgaagcagaaacaagaagaaatcaaagagaatataaagaatagttctgtcccaagaagaactctgaagatgattcagccttctgcatctggatctcttgttggaagagaaaatgagctgtccgcaggcttgtccaaaaggaaacatcggaatgaccacttaacatctacaacttccagccctggggttattgtcccagaatctagtgaaaataaaaatcttggaggagtcacc

SEQ ID NO: 4 - DNA, 618 bp, Homo sapiens
  misc_feature 33: n = A,T,C or G
gtaccgggcccccccctcggaaatacttctagnattaattaacgcgggtgaagaaaaaaaacgacttgaggaaaaacaaagagcagcccgcaaaaacaggtccaagtcagaagaggactggaagacgaggtggttccatcaaggtcctaatccctacaatggagcacaggactggatttactctggcagctactgggacagaaattacttcaatttgcctgacatttattaaaatgcatacaagtcagggtgtttggctaatctacaaataagtcttaaacctatgtttttaaatttttttcccttggtttctacttatcttttaaaaaaaaaatgaaaaaacactcatgagataactgcatttcacccaacaaaagcagggtataaggcgatattggtgatgaaagtcttaggaaaaatgcataattttgctataaaatgtacttatttggaatactattttatatagaggtaagagaacactgctggggaatatgctttttatggttgctgttgccatatttactgaaggtttatacctaaatgtaactttagctttatggaactatatagtaatcccaaatcaagttattttgaatatttttatgctgtcatgct

SEQ ID NO: 5 - DNA, 245 bp, Homo sapiens
atgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacaagtttgaacgacataacagc

SEQ ID NO: 6 - DNA, 567 bp, Homo sapiens
ccgggccccccctcggaagtcttctagaattaattaacgcgggtatattgtggcatatagtcaacatggaagtagaccagctctgctgatttgaaatttagattttttaaattatgtactggggacaggtttttgtcgctttacattgcttcctagtttacagcatgatgcaaatgattttctaacttagtgttaggagaaattattttccatctttaacctcttagttgtctaagagttaaatattactgaatttcagacgttcaaattgatcatcacaaatcctttaaaacaattacctaaaagaaaccaaaaatcctgccttctttgtgggggaggggggagagaggggaaggaaatggaacaagttgtgtttgtgttagcatgtgggtgatgtaaacttcaaattgggagatgttccgacccccactcccatataagcattcaatcagtgtaacttgcaaaatgcataaacatccgacagtttgaatttatagtgttgatggaagaaatcatttttaatgtgtactgtaaaacttgaaatactcaggagctgagtgaatatgt

SEQ ID NO: 7 - DNA, 598 bp, Homo sapiens
  misc_feature 24: n = A,T,C or G
gccccccctcggaagtcttctagnattaattaacgcgggtttacatacagatggcaaacttcatttcctttttctcttaatgcaacaaggtcatcccaagatcaggcttccttcagtttctgtggtaagtagtgatggacacttatggagttttcagagacttatgcattgggtaacaaggcactgcaagaaaccccagatagcacagcatcatctcacatttacaccacatcacatcaacatcgatgctaggaggtctaaagctgatgccaccttcagagctgcaagtatccaaaagactccactcatgacaccagctcactcctgagtcacatcagcccctttcagagtcacagccccagaactaaggctatccatatgggttgattataaaatccaaaaagcagctccaaatattagaaacacttatttgtctggcagacacattccctaatttaaagcctcttatactgtgcttaaaaatattcaacagtcttagcaaaatgacaaaataaacaggtagcagccatggggtgggcagggtaaggtggcgttgaaagaaaaggcatttagcaaggtaagtagagagtgctgatgg

SEQ ID NO: 8 - DNA, 555 bp, Homo sapiens
  misc_feature 28: n = A,T,C or G
ccgggccccccctcgaaatacttctagnattaattaacgcggggggggaagccatctgccttcgagcctgccactgaaatgcaaaagtctgtcccaaataaagccttggaattgaaaaatgaacaaacattgagagcagatgagatactcccatcagaatccaaacaaaaggactatgaagaaaattcttgggatactgagagtctctgtgagactgtttcacagaaggatgtgtgtttacccaaggctgcgcatcaaaaagaaatagataaaataaatggaaaattagaagggtctcctgttaaagatggtcttctgaaggctaactgcggaatgaaagtttctattccaactaaagccttagaattgatggacatgcaaactttcaaagcagagcctcccgagaagccatctgccttcgagcctgccattgaaatgcaaaagtctgttccaaataaagccttggaattgaagaatgaacaaacattgagagcagatgagatactcccatcagaaatcaaacaaaggactatgaagaaagttcttgggattctg

SEQ ID NO: 9 - DNA, 678 bp, Homo sapiens
  misc_feature 623, 641: n = A,T,C or G
acagttcataaggcctttgtcattgttagtcaccttcgatgtgaatatcagtagggccattaaattaaagctggcctaaaaagttttcaaagtgctactgtttccaactgatgaaatcttcagttttctggattttctgggtgacgattattttcaattctttttttctggtgatatatacaagaagttatagcagatatataaagggaagatcagaagcctgctgtccaagttcatcaccacttgttcctgatccttttcaagtgtaagtatttgatagccagttgttctactacatattagttttccactactatcaaaagaagccaaacgtaatctaaatgctatgctccttcgaggctgtaaactgacagatccgctacatggctgattcagtgtattgcgtttgaaaatgatgtatcgattggtgtaagttacaagtaggtcgaagccgaagttaaaacctgtccaacgccagcagtattcaccatctttggcaagctttctaccacacctcatgctgtttccctctagtttcttctttattgatttcttgaggccccacctcactgtcctgttctgcccgcagcatagcaaaccactgctgtttatacacagaagangagccattcttgaaaggnacctccggcccgtctagaactaggtggaatccccccg

SEQ ID NO: 10 - DNA, 544 bp, Homo sapiens
  misc_feature 3, 23, 28, 46, 266, 291, 296, 329, 376, 382, 389,391, 392, 393, 394, 396, 432, 440: n = A,T,C or G
ccnccgcgggggcggccgaggtnccttncattggattgtccatggngcatcgctttagcaagcggcgtgactctccactgcccgtcatcttggccaatatctatctgctggttcctcctgtgctcaacccaattgtctatggagtgaagacaaaggagattcgacagcgcatccttcgacttttccatgtggccacacacgcttcagaaccctaggtgtcagtgatcaaacttcttttccattcagagtcctctgattcagatttnaatgttaacattgtggaagacagtnttcanaaaaaaaatttccttaataaaaatacaactcanatccttcaaatatgaaactggttggggaatctccattttttcaatantatttncttcttngnnnncntgctacatataattattaataccctgactaggttgnggttgganggttattacttttgattttaccatgcagtccaaatctaaactgcttctactgatggtttacagcattctgagataagaatggtcatctagagaacatttgccaa

SEQ ID NO: 11 - DNA, 382 bp, Homo sapiens
  misc_feature 22, 52: n = A,T,C or G
actccaccgcggtggcggccgnggtaccatggtacaaaccagcatgccgggnaacacttgtcctctgtttgcctcaaataaagattattagtgctgggcacaagtatatggaacctctgcaggagattccatttgttatcccacgacccatccttgaagaaggtgatgcttttccttggacgatcagcttgcataatttcagcatatatacccttcttggaaaacaagtgacactttgcctagtggaacctatgggttgcacctccactctagctgtcacgtctcaaaaactgcttgctacgggacctgatacacgacattcatttgttgtctgtctccatgttgacctagagtcactagagataaaatgctctaatcccca

SEQ ID NO: 12 - DNA, 574 bp, Homo sapiens
  misc_feature 28, 34: n = A,T,C or G
ccgcggtggcggccgaggtacctccagnacaggnaggatctgccagttactacttggaacctcctcccttctctcttccctgtatgatgtattttctttcatggcttagtggtagctcaaagctcaaggtgaccagaatgattcccttacagcagtggttcccaaacattgatgtgcaacagaacaacttgggtagtttgtagaaagggcagatttcctggccctattcccagagattcggaattagtaggtttgaggtggggcccaggaatctgcactcaaatgtgctactctcaggatctgacctaaggggcctttcatatgtggccccctttcctcaccggaggacataagtgcatcacacaaacctcttgctgtgggcacttcctttggggttaaaaatgggaccatttgattaaggcaaccattgctaaccatcttacttccctgggctggagaaatatcaaatgtcacattttatgttcttaaaatctcagggccacagatttcctttgacaatgacatgctctgtaacatgaaatttatatcctaaagccttttgtccttccttctt

SEQ ID NO: 13 - DNA, 536 bp, Homo sapiens
  misc_feature 441, 443, 455, 479, 486, 488, 492, 502, 508, 514,516, 524, 529: n = A,T,C or G
acaggtccccggaggcatcctggctgggtgggaagtttctggcggtcacgccctgtccgctttcgctccaggtcacactgagtggctcctgggggaagaagccctggaccaggcaggcgatgaccacgttcccatctggctgggtgctgcagaggctcagcgggaagaccttggggctggtcggggatgctgaggagacggtgaccagagttccctggccccaggggtcaatccactgttcgtcaaaatactcggcttttgccgcacagtgatatagagccgtgtctgcagcggtcacagaggtcaacttcagggagaactgattcttggacgtgtcaaaggacatggtgactcgactcttgagggacgggttgtggaaaatactcccataatatgcgatggtcccaacccactccaggcccttccttggggggcttggcngnatccacccccanaaggaaccactactggtaatgganacaccananacantgcaagtganggacanggtctncnaaggcttnaccantcctggg

SEQ ID NO: 14 - DNA, 266 bp, Homo sapiens
acctttttttttttttttttttttttggggaagggggggtaaacccaaaaccctttttaaatgggggggttttttaacccccacatggggggaaaaatttttaccctttttaaaaaggttttttccaaaggcaaaaaaaactttccttttttgaacaaaaattaaatttcaaagggggttttaaggggttttggggccaaattaaaagggaaacaaaaatttttttttgaaaaaccatttttcaccagggtgggggggtttgcccc

SEQ ID NO: 15 - DNA, 301 bp, Homo sapiens
acgcggggtgggaaataaatcagggcggcctttggcatttcacaggcataactcctctattcttctgctgcaaatttagagcttacaagatgctaatggcacctcagtttccaggatagtctctgcagactgctttcactcagcaggcttcctttcatgatggctgccagtaacagctacccctcatttacatccagcagagatgaggggtatctcttcggggagttcgtgcaaaaaaaagcaagaaaagctgaaatcttctcacttcaaaggaatggcacatgacagcacttagccaata

SEQ ID NO: 16 - DNA, 27 bp, Homo sapiens
tccggcgcgccgttttcccagtcacga

(19) United States
(12) Patent Application Publication
(10) Pub. No.: US 2004/0009481 A1
(43) Pub. Date: Jan. 15, 2004

(54) Compositions, kits, and methods for identification, assessment, prevention, and therapy of human prostate cancer

(75) Inventors: Robert Schlegel, Auburndale, MA (US); Wilson O. Endege, Norwood, MA (US)

Correspondence Address: LAHIVE & COCKFIELD, 28 STATE STREET, BOSTON, MA, 02109, US

(73) Assignee: Millennium Pharmaceuticals, Inc., Cambridge, MA

(21) Appl. No.: 10/166,883

(22) Filed: Jun. 11, 2002

Related U.S. Application Data
(60) Provisional application No. 60/297,285, filed on Jun. 11, 2001.

(51) Int. Cl. C12Q 1/68; G01N 33/574

(52) U.S. Cl. 435/6; 435/7.23

(57) ABSTRACT

The invention relates to compositions, kits, and methods for diagnosing, staging, prognosing, monitoring and treating human prostate cancers. A variety of marker genes are provided, wherein changes in the levels of expression of one or more of the marker genes is correlated with the presence of prostate cancer.

### RELATED APPLICATIONS

The present application claims priority to U.S. provisional patent application serial No. 60/297,285, filed on Jun. 11, 2001, which is expressly incorporated by reference.

### FIELD OF THE INVENTION

The field of the invention is prostate cancer, including diagnosis, characterization, management, and therapy of prostate cancer.

### BACKGROUND OF THE INVENTION

The increased number of cancer cases reported in the United States, and, indeed, around the world, is a major concern. Currently there are only a handful of treatments available for specific types of cancer, and these provide no absolute guarantee of success. In order to be most effective, these treatments require not only an early detection of the malignancy, but also a reliable assessment of the severity of the malignancy.

Carcinoma of the prostate (PCA) is the most frequently diagnosed cancer in men in the United States, and is the second leading cause of male cancer deaths (Karp et al., 1996, Cancer Res. 56:5547-5556). The acute susceptibility of this organ to cancer in men is not understood. Skenes glands represent a tissue in females that is homologous to the male prostate, but not a site where significant neoplastic transformation is observed.

An unusual challenge presented by prostate cancer is that most prostate tumors do not represent life threatening conditions. Projections from autopsy surveys indicate that as many as 11 million American men have prostate cancer (Dhom, 1983, J. Cancer Res. Clin. Oncol., 106:210-218). These figures are consistent with clinical observations of prostate carcinomas, which normally exhibit a slow and lingering course of progression. Such disease progression results in relatively few prostate tumors developing into cases of clinical concern during the lifetime of the patient. If, upon detection with available methods, the cancer appears well-differentiated, organ-confined and focal, treatment normally can not extend the life expectancy of older patients.

Unfortunately, the prostate carcinomas that are progressive in nature frequently have already metastasized by the time of clinical detection with available methods. Survival rates for individuals with metastatic prostate cancer are quite low. Between these two extremes are patients with prostate tumors that will metastasize during their lifetimes, but have not yet done so. For these patients, surgical removal of the prostate is curative and extends life expectancy. Therefore, accurate determination of which group a newly diagnosed patient falls into is critical in determining optimal treatment and patient survival.

Currently there is at least one early and noninvasive test available to the physician for detecting a symptomatic disease. The presence of Prostate Specific Antigen (PSA) can be measured with relative ease from blood samples using standard antibody-based detection kits. Abnormally high levels of this antigen in a patient's serum indicate a likelihood of prostate disease, possibly either a carcinoma, Benign Prostatic Hyperplasia (BPH) or prostatitis. In the majority of cases, PSA elevation is due to BPH or prostatitis rather than carcinoma.

Although clinical and pathologic stage and histological grading systems (e.g., Gleason's) have been used to indicate prognosis for groups of patients based on the degree of tumor differentiation or the type of glandular pattern (Carter and Coffey, In: J. P. Karr and H. Yamanak (eds.), Prostate Cancer: The Second Tokyo Symposium, pp. 19-27, New York: Elsevier, 1989.; Diamond et al., J. Urol., 128: 729-734, 1982), these systems do not adequately predict the progression rate of the cancer. While the use of computer-system image analysis of histologic sections of primary lesions for “nuclear roundness” has been suggested as an aide in the management of individual patients (Diamond et al., 1982, J. Urol., 128:729-734), this method is of limited use in studying the progression of the disease.

The analysis of DNA content/ploidy using flow cytometry and FISH has been demonstrated to have utility predicting prostate cancer aggressiveness (Pearsons et al., 1993, J. Urol., 150:120-125; Macoska et al., 1994, Cancer Res., 54: 3824-3830; Visakorpi et al., 1994, Am. J. Pathol., 145:1-7; Takahashi et al., 1994, Cancer Res., 54:3574-3579; Alcaraz et al., Cancer Res., 55:3998-4002, 1994), but these methods are expensive, time-consuming, and the latter methodology requires the construction of centromere-specific probes for analysis. There also exist specific nuclear matrix proteins whose expression has been reported to be associated with prostate cancer. However, these protein markers apparently do not distinguish between BPH and prostate cancer (Partin et al., 1993, Cancer Res., 53:744-746). Unfortunately, markers that cannot distinguish between benign and malignant prostate tumors are of little value.

It would therefore be beneficial to provide specific methods and reagents for the diagnosis, staging, prognosis, monitoring, and treatment of diseases associated with prostate cancer, or to indicate a predisposition to such for preventative medicine.

### SUMMARY OF THE INVENTION

The invention relates to various methods, reagents and kits for diagnosing, staging prognosing, monitoring and treating prostate cancer. The methods of the present invention comprise comparing the level of expression of a single or plurality (e.g. 2, 3, 5, or 10 or more) of prostate cancer marker genes (hereinafter “marker genes”) in a patient sample, wherein the marker genes are listed within Tables 1-3D, and the control level of expression of the marker gene(s) in a sample from a control subject (e.g., a human subject without prostate cancer). In preferred embodiments, the control level of expression is the average level of expression of the marker gene(s) in samples from several (e.g., 2, 3, 4, 5, 8, 10, 12, 15, 20, 30 or 50) control subjects. A significant change in the level of expression of one or more of the marker genes in the patient sample relative to the control level provides significant information regarding the patient's prostate cancer status.

In one embodiment of the methods of the present invention, the sample comprises cells obtained from the patient. The cells may be found in a prostate tissue sample collected, for example, by a prostate tissue biopsy or histology section, or a bone marrow biopsy. In another embodiment, the patient sample is a prostate-associated body fluid. Such fluids include, for example, blood fluids, lymph, urine, prostatic fluid and semen.

In accordance with the methods of the present invention, the presence and/or level of expression of the marker gene in a sample can be assessed, for example, by detecting the presence in the sample of:

a protein encoded by the marker gene (e.g. using a reagent, such as an antibody, an antibody derivative, or an antibody fragment, which binds specifically with the protein or protein fragment) or a fragment of the protein.

a metabolite which is produced directly (i.e., catalyzed) or indirectly by a protein encoded by the marker gene

a transcribed polynucleotide (e.g. an mRNA or a cDNA), or fragment thereof, having at least a portion with which the marker gene is substantially homologous (e.g. by contacting a mixture of transcribed polynucleotides obtained from the sample with a substrate having a sequence of one or more of the marker genes listed within Tables 1-3D fixed thereto at selected positions)

a transcribed polynucleotide or fragment thereof, wherein the polynucleotide anneals with the marker gene under stringent hybridization conditions.

The methods of the present invention are useful for further diagnosing patients having an identified prostate mass or symptoms associated with prostate cancer, e.g. abnormally high levels of PSA. The methods of the present invention can be of special use in identifying the metastatic potential of prostate cancer in a patient or the specific type of metastatic prostate cancer, e.g., metastasis to the liver, bones or lymph nodes. The methods of the present invention can further be of particular use with patients having an enhanced risk of developing prostate cancer (e.g., patients having a familial history of prostate cancer and patients identified as having a mutant oncogene). The methods of the present invention may further be of particular use in monitoring the efficacy of treatment of a prostate cancer patient (e.g. the efficacy of chemotherapy).

All cancers have staging schemes that are used to describe the degree to which the cancer has progressed. The TNM staging approach assigns the primary tumor (T) to one of four stages (and to additional substages within these categories) based on the size and location of the primary tumor within the prostate. A T1 designation indicates a microscopic tumor which cannot be detected by a digital rectal exam. A T2NO designation refers to a tumor palpable upon a digital rectal exam but are contained within the prostate capsule (local disease). In all forms of stage T3 disease the tumors have extended through the prostate capsule into the surrounding connective tissue or seminal vesicles. The T4 designation refers to tumors that have escaped from the prostate and can be found in the pelvic region. The N stage refers to whether the primary tumor has spread to the regional lymph nodes (pelvic lymph nodes). The M stage refers to whether the tumor cells have metastasized to distant sites. The marker genes of the present invention are particularly useful in identifying whether prostate cancer has metastasized or is likely to metastasize. In particular, the marker genes set forth in Table 3A can be used to determine whether prostate cancer has metastasized, or is likely to metastasize, to the liver (M stage). The marker genes set forth in Table 3B can be used to determine whether prostate cancer has metastasized, or is likely to metastasize, to the bone (M stage). The marker genes set forth in Tables 3C and 3D can be used to determine whether prostate cancer has metastasized, or is likely to metastasize, to the lymph nodes (N stage and/or M stage).

The methods of the present invention may be performed using a plurality (e.g. 2, 3, 5, or 10 or more) of marker genes. According to a method involving a plurality of marker genes, the level of expression in the sample of each of a plurality of marker genes independently selected from the marker genes listed in Tables 1-3D is compared with the normal level of expression of each of the plurality of marker genes in samples of the same type obtained from control human subjects not afflicted with prostate cancer. A significantly increased level of expression in the sample of one or more of the marker genes listed in Tables 1-3D, or some combination thereof, relative to that marker gene's corresponding normal levels, is an indication that the patient is afflicted with prostate cancer. The marker genes of Tables 1-3D may also be used in combination with known prostate cancer marker genes in the methods of the present invention, e.g. PSA analysis.

In a method of assessing whether a patient is afflicted with prostate cancer (e.g., new detection (“screening”), detection of recurrence, reflex testing), the method comprises comparing:

a) the level of expression of a single or plurality of marker genes in a patient sample, wherein at least one marker gene is selected from the marker genes of Tables 1-3D, and

b) the normal level of expression of the same marker gene(s) in a sample from a control subject having no prostate cancer.

A significant increase in the level of expression of one or more of the marker genes in the patient sample, relative to the normal level is an indication that the patient is afflicted with prostate cancer.

The present invention further includes a method for determining whether prostate cancer has metastasized or is likely to metastasize in the future, the method comprising comparing:

a) the level of expression of a single or plurality of marker genes in a patient sample, wherein at least one marker gene is selected from the marker genes of Tables 1-3D and

b) the level of expression of the same marker gene(s) in a sample from a control subject having non-metastasized prostate cancer.

A significantly increased level of expression of one or more of the marker genes in the patient sample, relative to the level of expression in the control, is an indication that the patient is afflicted with metastatic prostate cancer that has metastasized, or is likely to metastasize in the future.

The present invention further includes a method for determining whether prostate cancer has metastasized to the liver, or is likely to metastasize to the liver, the method comprising comparing:

a) the level of expression of a single or plurality of marker genes in a patient sample, wherein at least one such marker gene is selected from marker genes 1734-3683 of Table 1 or the marker genes of Table 3A and

b) the level of expression of the same marker gene(s) in a sample from a control subject having non-metastasized prostate cancer.

A significantly increased level of expression of one or more of the marker genes in the patient sample, relative to the level of expression in the control, is an indication that the patient is afflicted with metastatic prostate cancer that has metastasized to the liver, or is likely to metastasize to the liver.

In another such embodiment, the present invention includes a method for determining whether prostate cancer has metastasized to bone tissue, or is likely to metastasize to bone tissue, the method comprising comparing:

a) the level of expression of a single or plurality of marker genes in a patient sample, wherein at least one such marker gene is selected from marker genes 3684-5660 of Table 1 or the marker genes of Table 3B and

b) the level of expression of the same marker gene(s) in a sample from a control subject having non-metastasized prostate cancer.

A significantly increased level of expression of one or more of the marker genes in the patient sample, relative to the level of expression in the control, is an indication that the patient is afflicted with metastatic prostate cancer that has metastasized to bone tissue, or is likely to metastasize to bone tissue.

In another such embodiment, the present invention includes a method for determining whether prostate cancer has metastasized to lymph nodes, or is likely to metastasize to lymph nodes, the method comprising comparing:

a) the level of expression of a single or plurality of marker genes in a patient sample, wherein at least one such marker gene is selected from marker genes 1-1733 and 5661-11617 of Table 1 or the marker genes of Tables 3C or 3D, and

b) the level of expression of the same marker gene(s) in a sample from a control subject having non-metastasized prostate cancer.

A significantly increased level of expression of one or more of the marker genes in the patient sample, relative to the level of expression in the control, is an indication that the patient is afflicted with metastatic prostate cancer that has metastasized to lymph nodes, or is likely to metastasize to lymph nodes.

The present invention also includes a method for assessing the aggressiveness of prostate cancer, the method comprising comparing:

a) the level of expression of a single or plurality of marker genes in a patient sample, wherein at least one such marker gene is selected from the marker genes listed in Tables 1-3D, and

b) the level of expression of the same marker gene(s) in a control sample from a subject having non-metastasized prostate cancer.

A significantly increased level of expression of one or more of the marker genes in the patient sample, relative to the level in the control sample, is an indication that the patient is afflicted with an aggressive prostate cancer.

The present invention also includes a method for assessing the indolence of prostate cancer, the method comprising comparing:

a) the level of expression of a single or plurality of marker genes in a patient sample, wherein at least one such marker gene is selected from the marker genes listed in Tables 1-3D, and

b) the level of expression of the same marker gene(s) in a control sample from a subject having non-metastasized prostate cancer.

A significantly decreased level of expression of one or more of the marker genes in the patient sample, relative to the level in the control sample, is an indication that the patient is afflicted with an indolent prostate cancer.

The invention further relates to a method of assessing the efficacy of a therapy for inhibiting prostate cancer in a patient. This method comprises comparing:

a) the level of expression of a single or plurality of marker genes in a first sample obtained from the patient prior to providing at least a portion of the therapy to the patient, wherein at least one marker gene(s) is selected from the group consisting of the marker genes listed within Tables 1-3D, and

b) the level of expression of the same marker gene(s) in a second sample obtained from the patient following provision of the portion of the therapy.

A significant decreased level of expression of one or more of the marker genes in the second sample, relative to the level in the first sample, is an indication that the therapy is efficacious for inhibiting prostate cancer in the patient.

It will be appreciated that in this method the “therapy” may be any therapy for treating prostate cancer including, but not limited to, chemotherapy, immunotherapy, gene therapy, radiation therapy and surgical removal of tissue. Thus, the methods of the invention may be used to evaluate a patient before, during and after therapy, for example, to evaluate the reduction in tumor burden.

The present invention therefore further comprises a method for monitoring the progression of prostate cancer in a patient, the method comprising:

a) detecting in a patient sample at a first time point, the expression of a single or plurality of marker genes, wherein at least one of the marker genes is selected from the group consisting of the marker genes listed in Tables 1-3D;

b) repeating step a) at a subsequent time point in time; and

c) comparing the level of expression of the same marker gene(s) detected in steps a) and b), and therefrom monitoring the progression of prostate cancer in the patient.

The invention also includes a method of selecting a composition for inhibiting prostate cancer in a patient. This method comprises the steps of:

a) obtaining a sample comprising cancer cells from the patient;

b) separately maintaining aliquots of the sample in the presence of a plurality of test compositions;

c) comparing expression of a single or plurality of marker genes listed within Tables 1-3D in each of the aliquots; and

d) selecting one of the test compositions which decreases the level of expression of one or more of the marker genes in the aliquot containing that test composition, relative to other test compositions.

In addition, the invention includes a method of inhibiting prostate cancer in a patient. This method comprises the steps of:

a) obtaining a sample comprising cancer cells from the patient;

b) separately maintaining aliquots of the sample in the presence of a plurality of test compositions;

c) comparing expression of a single or plurality of marker genes listed within Tables 1-3D in each of the aliquots; and

d) administering to the patient at least one of the test compositions which decreases the level of expression of one or more of the marker genes in the aliquot containing that test composition, relative to other test compositions.

The invention also includes a kit for assessing whether a patient is afflicted with prostate cancer. This kit comprises reagents for assessing expression of a marker gene listed within Tables 1-3D.

In another aspect, the invention also relates to a kit for assessing the specific type of metastatic prostate cancer, e.g., cancer that has metastasized to the liver, bone or lymph nodes. For liver metastasis, the kit may comprise reagents for assessing the expression of any of the marker genes 1734-3683 of Table 1 and the marker genes of Table 3A. For bone metastasis, the kit may comprise reagents for assessing the expression of any of the marker genes 3684-5660 of Table 1 and the marker genes of Table 3B. For metastasis to lymph nodes, the kit may comprise reagents for assessing the expression of any of the marker genes 1-1773 and 5661-11617 of Table 1 and the marker genes of Tables 3C-3D.

In another aspect, the invention relates to a kit for assessing the suitability of each of a plurality of compounds for inhibiting a prostate cancer in a patient. The kit comprises a reagent for assessing expression of a marker gene listed within Tables 1-3D, and may also comprise a plurality of compounds.

In another aspect, the invention relates to a kit for assessing the presence of prostate cancer cells. This kit comprises an antibody, wherein the antibody binds specifically with a protein or protein fragment corresponding to a marker gene listed within Tables 1-3D. The kit may also comprise a plurality of antibodies, wherein the plurality binds specifically with a protein or protein fragment corresponding to a different marker gene listed within Tables 1-3D.

The invention also includes a kit for assessing the presence of prostate cancer cells, wherein the kit comprises a nucleic acid probe. The probe binds specifically with a transcribed polynucleotide corresponding to a marker gene listed within Tables 1-3D. The kit may also comprise a plurality of probes, wherein each of the probes binds specifically with a transcribed polynucleotide corresponding to a different marker gene listed within Tables 1-3D.

The invention further relates to a method of making an isolated hybridoma which produces an antibody useful for assessing whether a patient is afflicted with prostate cancer. The method comprises isolating a protein or protein fragment corresponding to a marker gene listed within Tables 1-3D, immunizing a mammal using the isolated protein or protein fragment, isolating splenocytes from the immunized mammal, fusing the isolated splenocytes with an immortalized cell line to form hybridomas, and screening individual hybridomas for production of an antibody which specifically binds with the protein or protein fragment, to isolate the hybridoma. The invention also includes an antibody produced by this method.

The invention further includes a method of assessing the prostate carcinogenic potential of a test compound. This method comprises the steps of:

a) maintaining separate aliquots of prostate cells in the presence and absence of the test compound; and

b) comparing the level of expression of a single or plurality of marker genes in each of the aliquots, wherein at least one of the marker genes is selected from those listed within Tables 1-3D. A significant increase in the level of expression of one or more of the marker genes in the aliquot maintained in the presence of (or exposed to) the test compound, relative to the level of expression of the same marker gene(s) in the aliquot maintained in the absence of the test compound, is an indication that the test compound possesses prostate carcinogenic potential.

Additionally, the invention includes a kit for assessing the prostate carcinogenic potential of a test compound. The kit comprises prostate cells and a reagent for assessing expression of a single or plurality of marker genes in each of the aliquots, wherein at least one of the marker genes is selected from those listed within Tables 1-3D.

The invention further relates to a method of treating a patient afflicted with prostate cancer. This method comprises providing to cells of the patient an antisense oligonucleotide complementary to a polynucleotide corresponding to a marker gene listed within Tables 1-3D, which is over-expressed in prostate cancer cells.

The invention includes a method of inhibiting prostate cancer in a patient at risk for developing prostate cancer. This method comprises inhibiting expression (or overexpression) of a marker gene listed within Tables 1-3D.

It will be appreciated that the methods and kits of the present invention may also include known cancer marker genes including known prostate cancer marker genes. It will further be appreciated that the methods and kits may be used to identify cancers other than prostate cancer.

### DETAILED DESCRIPTION OF THE INVENTION

The invention relates to newly discovered correlations between expression of certain marker genes and the cancerous state of prostate cells. It has been discovered that the over expression of individual marker genes and combinations of marker genes described herein correlates with the presence of prostate cancer or the metastatic status and/or potential of such a cancer, or the specific type of metastatic prostate cancer in a patient. Methods are provided for detecting the presence of prostate cancer in a sample, the absence of prostate cancer in a sample, the metastatic potential of a prostate cancer, the specific type of metastic prostate cancer, e.g., metastasis to the liver, bone or lymph nodes, the stage of a prostate cancer, the indolence or aggressiveness of the cancer, and other characteristics of prostate cancer that are relevant to prevention, diagnosis, characterization and therapy of prostate cancer in a patient.

Definitions

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

A “marker gene” is a gene whose altered level of expression in a tissue or cell from its expression level in normal or healthy tissue or cell is associated with a disease state, such as prostate cancer. A “marker nucleic acid” is a nucleic acid (e.g., mRNA, cDNA) encoded by or corresponding to a marker gene of the invention. Such marker nucleic acids can be DNA (e.g., cDNA) comprising the sequence of any of the sequences of Table 1 or the complement of such sequences. The marker nucleic acids also can be RNA comprising the sequence of any of the sequences of Table 1 or the complement of such sequence, wherein all thymidine residues are replaced with uridine residues. A “marker protein” is a protein encoded by or corresponding to a marker gene of the invention. The terms “protein” and “polypeptide” are used interchangeably.

As used herein a polynucleotide “corresponds to” another (a first) polynucleotide if it is related to the first polynucleotide by any of the following relationships: The second polynucleotide comprises the first polynucleotide and the second polynucleotide encodes a gene product; 2) The second polynucleotide is 5′ or 3′ to the first polynucleotide in cDNA, RNA, genomic DANA, or fragment of any of these polynucleotides. For example, a second polynucleotide may be a fragment of a gene that includes the first and second polynucleotides. The first and second polynucleotides are related in that they are components of the gene coding for a gene product, such as a protein or antibody. However, it is not necessary that the second polynucleotide comprises or overlaps with the first polynucleotide to be encompassed within the definition of “corresponding to” as used herein. For example, the first polynucleotide may be a fragment of a 3′ untranslated region of the second polynucleotide. The first and second polynucleotide may be fragments of a gene coding for a gene product. The second polynucleotide may be an exon of the gene while the first polynucleotide may be an intron of the gene; 3) The second polynucleotide is the complement of the first polynucleotide.

The term “probe” refers to any molecule which is capable of selectively binding to a specifically intended target molecule, for example a marker gene of the invention. Probes can be either synthesized by one skilled in the art, or derived from appropriate biological preparations. For purposes of detection of the target molecule, probes may be specifically designed to be labeled, as described herein. Examples of molecules that can be utilized as probes include, but are not limited to, RNA, DNA, cDNA, proteins, antibodies, and organic monomers.

A “prostate-associated” body fluid is a fluid which, when in the body of a patient, contacts or passes through prostate cells or into which cells or proteins shed from prostate cells are capable of passing. Exemplary prostate-associated body fluids include blood fluids, semen, prostate fluid, lymph and urine.

The “normal” level of expression of a marker gene is the level of expression of the marker gene in prostate cells or prostate-associated body fluids of a patient, e.g. a human, not afflicted with prostate cancer.

“Over-expression” of a marker gene refers to expression of the marker gene of a patient at a greater level, respectively, than the normal level of expression of the marker gene (e.g. at least two-fold greater or lesser level).

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue-specific manner.

A “constitutive” promoter is a nucleotide sequence which, when operably linked with a polynucleotide which encodes or specifies a gene product, causes the gene product to be produced in a living human cell under most or all physiological conditions of the cell.

An “inducible” promoter is a nucleotide sequence which, when operably linked with a polynucleotide which encodes or specifies a gene product, causes the gene product to be produced in a living human cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living human cell substantially only if the cell is a cell of the tissue type corresponding to the promoter.

A “transcribed polynucleotide” is a polynucleotide (e.g. an RNA, a cDNA, or an analog of one of an RNA or cDNA) which is complementary to or homologous with all or a portion of a mature RNA made by transcription of a genomic DNA corresponding to a marker gene of the invention and normal post-transcriptional processing (e.g. splicing), if any, of the transcript.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

“Homologous” as used herein, refers to nucleotide sequence similarity between two regions of the same nucleic acid strand or between regions of two different nucleic acid strands. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

A marker gene is “fixed” to a substrate if it is covalently or non-covalently associated with the substrate such that the substrate can be rinsed with a fluid (e.g. standard saline citrate, pH 7.4) without a substantial fraction of the marker gene dissociating from the substrate.

As used herein, a “naturally-occurring” nucleic acid molecule refers to an RNA or DNA molecule having a nucleotide sequence that occurs in nature.

Expression of a marker gene in a patient is “significantly” higher than the control (e.g., normal) level of expression of a marker gene if the level of expression of the marker gene is greater than the control level by an amount greater than the standard error of the assay employed to assess expression, and preferably at least twice, and more preferably three, four, five or ten times that amount. Alternately, expression of the marker gene in the patient can be considered “significantly” higher or lower than the control level of expression if the level of expression is at least about two, and preferably at least about three, four, or five times, higher or lower, respectively, than the control level of expression of the marker gene.

Prostate cancer is “inhibited” if at least one symptom of the cancer is alleviated, terminated, slowed, or prevented. As used herein, prostate cancer is also “inhibited” if recurrence or metastasis of the cancer is reduced, slowed, delayed, or prevented.

A kit is any manufacture (e.g. a package or container) comprising at least one reagent, e.g. a probe, for specifically detecting a marker gene of the invention, the manufacture being promoted, distributed, or sold as a unit for performing the methods of the present invention.

Description

The present invention is based, in part, on identification of marker genes which are over-expressed in metastatic prostate cancer cells when compared with normal prostate cells and/or non-metastatic prostate cancer cells. The present invention is also based, in part, on identification of marker genes which are indicative of the specific type of metastatic prostate cancer e.g., metastasis to the liver, bone or lymph nodes.

The over-expression of one or more of these marker genes in metastasized prostate cells is herein correlated with the metastatic state of the prostate cancer. The invention thus includes compositions, kits, and methods for assessing the cancerous state of prostate cells (e.g. cells obtained from a human, cultured human cells, archived or preserved human cells and in vivo cells) and/or the metastatic state of prostate cancer.

The compositions, kits, and methods of the invention have the following uses, among others:

1) assessing whether a patient is afflicted with prostate cancer;

2) assessing the stage of prostate cancer in a human patient;

3) assessing the grade of prostate cancer in a patient;

4) assessing the benign or malignant nature of prostate cancer in a patient;

5) assessing the metastatic status and/or potential of prostate cancer in a patient;

6) identifying the specific type of metastatic prostate cancer, e.g., that metastasized to the liver, bone or lymph nodes.

7) assessing the histological type of neoplasm (e.g. Adenocarcinoma) associated with prostate cancer in a patient;

8) assessing the indolent or aggressive nature of prostate cancer in a patient;

9) making an isolated hybridoma which produces an antibody useful for assessing whether a patient is afflicted with prostate cancer;

10) assessing the presence of prostate cancer cells;

11) assessing the efficacy of one or more test compounds for inhibiting prostate cancer in a patient;

12) assessing the efficacy of a therapy for inhibiting prostate cancer in a patient;

13) monitoring the progression of prostate cancer in a patient;

14) selecting a composition or therapy for inhibiting prostate cancer in a patient;

15) treating a patient afflicted with prostate cancer;

16) inhibiting prostate cancer in a patient;

17) assessing the prostate carcinogenic potential of a test compound; and

18) inhibiting prostate cancer in a patient at risk for developing prostate cancer.

The invention thus includes a method of assessing whether a patient is afflicted with prostate cancer which includes assessing whether the patient has metastasized prostate cancer. This method comprises comparing the level of expression of a marker gene in a patient sample and the level of expression of the marker gene in a control, e.g., a non-metastasized prostate cancer sample. A significant increase between the level of expression of the marker gene in the patient sample and the control level is an indication that the patient is afflicted with metastasized prostate cancer. The marker gene is selected from the group consisting of the marker genes listed within Tables 1-3D. Although one or more molecules corresponding to the marker genes listed within Tables 1-3D may have been described by others, the significance of the level of expression of these marker genes with regard to the cancerous state of prostate cells, or specific type of metastatic cancer, has not previously been recognized.

The invention also encompasses polynucleotides which differ from that of the polynucleotides described herein, but which produce the same phenotypic effect, such as an allelic variant. These altered, but phenotypically equivalent polynucleotides are referred to as “equivalent nucleic acids.” This invention also encompasses polynucleotides characterized by changes in non-coding regions that do not alter the polypeptide produced therefrom when compared to the polynucleotide herein. This invention further encompasses polynucleotides, which hybridize to the polynucleotides of the subject invention under conditions of moderate or high stringency. Alternatively, the polynucleotides are at least 85%, or at least 90%, or more preferably, greater or equal to 95% identical as determined by a sequence alignment program when run under default parameters.

Table 1 shows the accession number (“Ace. No.”) and corresponding GenBank GI number (“GI No.”) for the marker genes of the present invention that were identified through subtracted library experiments described herein, using metastatic prostate cancer samples as a source of RNA. Table 2 includes sequences, identified as SEQ ID NOS: 1-15, for the marker genes of Table 1, which are found in published international applications.

Tables 3A-3D list marker genes identified through transcriptional profiling, that were expressed at least 2-fold or greater in the following sample comparisons:

a) liver metastasis samples, i.e. prostate cancer that had metastasized to the liver, compared to primary prostate tumor samples of good clinical outcome (i.e. tumor samples that were not metastatic) and normal lymph nodes, normal liver and normal prostate samples (Table 3A, “Liver Mets”).

b) bone metastasis samples, i.e. prostate cancer that had metastasized to the bone, compared to primary prostate tumor samples of good clinical outcome and normal lymph nodes, normal liver and normal prostate samples (Table 3B, “Bone Mets”).

c) lymph node metastasis from two different sample sources, i.e. prostate cancer that had metastasized to the lymph nodes, compared to primary prostate tumor samples of good clinical outcome and normal lymph nodes and normal prostate samples (Table 3C, “Nodes 1” and Table 3D, “Nodes 2”).

Any marker gene or combination of marker genes listed within Tables 1-3D, as well as any known marker genes in combination with the marker genes set forth within Tables 1-3D, may be used in the compositions, kits, and methods of the present invention. In general, it is preferable to use marker genes for which the increase in the level of expression of the marker gene in prostate cancer cells or prostate-associated body fluids, as compared to the level of expression of the same marker gene in normal prostate cells or prostate-associated body fluids is as great as possible. Although this increase can be as small as the limit of detection of the method for assessing expression of the marker gene, it is preferred that the increase be at least greater than the standard error of the assessment method, and preferably an increase of at least 2-, 3-, 4-, 5-, 6-, 7-, 8-, 9-, 10-, 15-, 20-, 25-, 100-, 500-, 1000-fold or greater.

It will be appreciated that patient samples containing prostate cells may be used in the methods of the present invention. In these embodiments, the level of expression of the marker gene can be assessed by assessing the amount (e.g. absolute amount or concentration) of the marker gene in a prostate cell sample, e.g., prostate tissue sample obtained from a patient. The cell sample can, of course, be subjected to a variety of well-known post-collection preparative and storage techniques (e.g. fixation, storage, freezing, lysis, homogenization, DNA or RNA extraction, ultrafiltration, concentration, evaporation, centrifugation, etc.) prior to assessing the amount of the marker gene in the sample.

It will also be appreciated that certain marker genes correspond to proteins which are secreted from prostate cells (i.e. one or both of normal and cancerous cells) to the extracellular space surrounding the cells. These marker genes are preferably used in certain embodiments of the compositions, kits, and methods of the invention, owing to the fact that the protein corresponding to each of these marker genes can be detected in a prostate-associated body fluid sample. In addition, preferred in vivo techniques for detection of a protein corresponding to a marker gene of the invention include introducing into a subject a labeled antibody directed against the protein. For example, the antibody can be labeled with a radioactive marker gene whose presence and location in a subject can be detected by standard imaging techniques.

Although not every marker gene corresponding to a secreted protein is indicated as such herein, it is a simple matter for the skilled artisan to determine whether any particular marker gene corresponds to a secreted protein. In order to make this determination, the protein corresponding to a marker gene is expressed in a test cell (e.g. a cell of a prostate cell line), extracellular fluid is collected, and the presence or absence of the protein in the extracellular fluid is assessed (e.g. using a labeled antibody which binds specifically with the protein).

The following is an example of a method which can be used to detect secretion of a protein corresponding to a marker gene of the invention. About 8×10⁵ 293T cells are incubated at 37° C. in wells containing growth medium (Dulbecco's modified Eagle's medium {DMEM} supplemented with 10% fetal bovine serum) under a 5% (v/v) CO₂, 95% air atmosphere to about 60-70% confluence. The cells are then transfected using a standard transfection mixture comprising 2 micrograms of DNA comprising an expression vector encoding the protein and 10 microliters of LipofectAMINE™ (GIBCO/BRL Catalog no. 18342-012) per well. The transfection mixture is maintained for about 5 hours, and then replaced with fresh growth medium and maintained in an air atmosphere. Each well is gently rinsed twice with DMEM which does not contain methionine or cysteine (DMEM-MC; ICN Catalog no. 16-424-54). About 1 milliliter of DMEM-MC and about 50 microcuries of Trans-³⁵S™ reagent (ICN Catalog no. 51006) are added to each well. The wells are maintained under the 5% CO₂ atmosphere described above and incubated at 37° C. for a selected period. Following incubation, 150 microliters of conditioned medium is removed and centrifuged to remove floating cells and debris. The presence of the protein in the supernatant is an indication that the protein is secreted.

Examples of prostate-associated body fluids include blood fluids (e.g. whole blood, blood serum, blood having platelets removed therefrom, lymph, urine, prostatic fluid and semen. Many prostate-associated body fluids (i.e. usually excluding urine) can have prostate cells therein, particularly when the prostate cells are cancerous, and, more particularly, when the prostate cancer is metastasizing. Cell-containing fluids which can contain prostate cancer cells include, but are not limited to, whole blood, blood having platelets removed therefrom, lymph, prostatic fluid, and semen. Thus, the compositions, kits, and methods of the invention can be used to detect expression of marker genes corresponding to proteins having at least one portion which is displayed on the surface of cells which express it. Although the proteins having at least one cell-surface portion are not set forth herein, it is a simple matter for the skilled artisan to determine whether the protein corresponding to any particular marker gene comprises a cell-surface protein. For example, immunological methods may be used to detect such proteins on whole cells, or well known computer-based sequence analysis methods (e.g. the SIGNALP program; Nielsen et al., 1997, Protein Engineering 10:1-6) may be used to predict the presence of at least one extracellular domain (i.e. including both secreted proteins and proteins having at least one cell-surface domain). Expression of a marker gene corresponding to a protein having at least one portion which is displayed on the surface of a cell which expresses it may be detected without necessarily lysing the cell (e.g. using a labeled antibody which binds specifically with a cell-surface domain of the protein).

Expression of a marker gene of the invention may be assessed by any of a wide variety of well known methods for detecting expression of a transcribed molecule or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

In another preferred embodiment, expression of a marker gene is assessed using an antibody (e.g. a radio-labeled, chromophore-labeled, fluorophore-labeled, or enzyme-labeled antibody), an antibody derivative (e.g. an antibody conjugated with a substrate or with the protein or ligand of a protein-ligand pair {e.g. biotin-streptavidin} or an antibody fragment (e.g. a single-chain antibody, an isolated antibody hypervariable domain, etc.) which binds specifically with a protein or protein fragment corresponding to the marker gene, such as the protein encoded by the open reading frame corresponding to the marker gene or such a protein which has undergone all or a portion of its normal post-translational modification.

In another preferred embodiment, expression of a marker gene is assessed by preparing mRNA/cDNA (i.e. a transcribed polynucleotide) from cells in a patient sample, and by hybridizing the mRNA/cDNA with a reference polynucleotide which is a complement of a polynucleotide comprising the marker gene, and fragments thereof. cDNA can, optionally, be amplified using any of a variety of polymerase chain reaction methods prior to hybridization with the reference polynucleotide. Expression of one or more marker genes can likewise be detected using quantitative PCR to assess the level of expression of the marker gene(s). Alternatively, any of the many known methods of detecting mutations or variants (e.g. single nucleotide polymorphisms, deletions, etc.) of a marker gene of the invention may be used to detect occurrence of a marker gene in a patient.

In a related embodiment, a mixture of transcribed polynucleotides obtained from the sample is contacted with a substrate having fixed thereto a polynucleotide complementary to or homologous with at least a portion (e.g. at least 7, 10, 15, 20, 25, 30, 40, 50, 100, 500, or more nucleotide residues) of a marker gene of the invention. If polynucleotides complementary to or homologous with a marker gene of the invention are differentially detectable on the substrate (e.g. detectable using radioactivity, different chromophores or fluorophores), are fixed to different selected positions, then the levels of expression of a plurality of marker genes can be assessed simultaneously using a single substrate (e.g. a “gene chip” microarray of polynucleotides fixed at selected positions). When a method of assessing marker gene expression is used which involves hybridization of one nucleic acid with another, it is preferred that the hybridization be performed under stringent hybridization conditions.

Because the compositions, kits, and methods of the invention rely on detection of an increase in expression levels of one or more marker genes of the invention, it is preferable that the level of expression of the marker gene is significantly greater than the minimum detection limit of the method used to assess expression in at least one of normal prostate cells and cancerous prostate cells.

It is understood that by routine screening of additional patient samples using one or more of the marker genes of the invention, it will be realized that certain of the marker genes are over- or underexpressed in cancers of various types, including specific prostate cancers, as well as other cancers such as ovarian cancers. For example, it will be confirmed that some of the marker genes of the invention are over-expressed in most (i.e. 50% or more) or substantially all (i.e. 80% or more) of prostate cancer. Furthermore, it will be confirmed that certain of the marker genes of the invention are associated with prostate cancer of various stages.

It will be appreciated that as a greater number of patient samples are assessed for expression of the marker genes of the invention and the outcomes of the individual patients from whom the samples were obtained are correlated, it will also be confirmed that increased expression of certain of the marker genes of the invention are strongly correlated with malignant cancers and that decreased expression of other marker genes of the invention are strongly correlated with benign tumors. It will also be confirmed that increased expression of certain of the marker genes of the invention are strongly correlated with specific types of metastatic prostate cancers (e.g., metastasis to the liver, bone or lymph nodes). The compositions, kits, and methods of the invention are thus useful for characterizing one or more of the stage, grade, histological type, metastatic type, metastatic potential, indolent vs. aggressive phenotype and benign/malignant nature of prostate cancer in patients.

When the compositions, kits, and methods of the invention are used for characterizing one or more of the stage, grade, histological type, metastatic potential, indolent vs. aggressive phenotype and benign/malignant nature of prostate cancer in a patient, it is preferred that the marker gene or panel of marker genes of the invention is selected such that a positive result is obtained in at least about 20%, and preferably at least about 40%, 60%, or 80%, and more preferably in substantially all patients afflicted with a prostate cancer of the corresponding stage, grade, histological type, metastatic potential, indolent vs. aggressive phenotype or benign/malignant nature. Preferably, the marker gene or panel of marker genes of the invention is selected such that a positive predictive value (PPV) of greater than about 10% is obtained for the general population.

When a plurality of marker genes of the invention are used in the compositions, kits, and methods of the invention, the level of expression of each marker gene in a patient sample can be compared with the normal level of expression of each of the plurality of marker genes in non-cancerous samples of the same type, either in a single reaction mixture (i.e. using reagents, such as different fluorescent probes, for each marker gene or a mixture of similarly labeled probes to access a plurality of marker genes that are fixed to a single substrate at different positions) or in individual reaction mixtures corresponding to one or more of the marker genes. In one embodiment, a significantly enhanced level of expression of more than one of the plurality of marker genes in the sample, relative to the corresponding normal levels, is an indication that the patient is afflicted with prostate cancer. When a plurality of marker genes is used, it is preferred that 2, 3, 4, 5, 8, 10, 12, 15, 20, 30, or 50 or more individual marker genes be used, wherein fewer marker genes are preferred.

In order to maximize the sensitivity of the compositions, kits, and methods of the invention (i.e. by interference attributable to cells of non-prostate origin in a patient sample), it is preferable that the marker gene of the invention used therein be a marker gene which has a restricted tissue distribution, e.g., normally not expressed in non-prostate tissue.

Only a small number of marker genes are known to be associated with prostate cancers (e.g. PSA, PSMA, PAP, PCA3, PCTA-1, PSCA and STEAP). These marker genes are not, of course, included among the marker genes of the invention, although they may be used together with one or more marker genes of the invention in a panel of marker genes, for example. It is well known that certain types of genes, such as oncogenes, tumor suppressor genes, growth factor-like genes, protease-like genes, and protein kinase-like genes are often involved with development of cancers of various types. Thus, among the marker genes of the invention, use of those which correspond to proteins which resemble known proteins encoded by known oncogenes and tumor suppressor genes, and those which correspond to proteins which resemble growth factors, proteases, and protein kinases are preferred.

Known oncogenes and tumor suppressor genes include, for example, abl, abr, akT2NO, apc, bcl2α, bcl2β, bcl3, bcr, brca1, brca2, cbl, ccnd1, cdc42, cdk4, crk-II, csflr/fms, dbl, dcc, dpc4/smad4, e-cad, e2f/rbap, egfr/erbb-1, elk1, elk3, eph, erg, ets1, ets2, fer, fgr/src2, fli1/ergb2, fos, fps/fes, fra1, fra2, fyn, hck, hek, her2/erbb-2/neu, her3/erbb-3, her4/erbb-4, hras1, hsT2NO, hstf1, igfbp2, ink4a, ink4b, inT2NO/fgf3, jun, junb, jund, kip2, kit, kras2a, kras2b, lck, lyn, mas, max, mcc, mdm2, met, mlh1, mmp10, mos, msh2, msh3, msh6, myb, myba, mybb, myc, mycl1, mycn, nf1, nf2, nme2, nras, p53, pdgfb, phb, pim1, pms1, pms2, ptc, pten, raf1, rap1a, rb1, rel, ret, ros1, ski, src1, tal1, tgfbr2, tgfb3, tgfbr3, thra1, thrb, tiam1, timp3, typ1, tp53, trk, vav, vh1, vil2, waf1, wnt1, wnT2NO, wt1, and yes1 (Hesketh, 1997, In: The Oncogene and Tumour Suppressor Gene Facts Book, 2nd Ed., Academic Press; Fishel et al., 1994, Science 266:1403-1405).

Known growth factors include platelet-derived growth factor alpha, platelet-derived growth factor beta (simian sarcoma viral {v-sis} oncogene homolog), o thrombopoietin (myeloproliferative leukemia virus oncogene ligand, megakaryocyte growth and development factor), erythropoietin, B cell growth factor, macrophage stimulating factor 1 (hepatocyte growth factor-like protein), hepatocyte growth factor (hepapoietin A), insulin-like growth factor 1 (somatomedia C), hepatoma-derived growth factor, amphiregulin (schwannoma-derived growth factor), bone morphogenetic proteins 1, 2, 3, 3 beta, and 4, bone morphogenetic protein 7 (osteogenic protein 1), bone morphogenetic protein 8 (osteogenic protein 2), connective tissue growth factor, connective tissue activation peptide 3, epidermal growth factor (EGF), teratocarcinoma-derived growth factor 1, endothelin, endothelin 2, endothelin 3, stromal cell-derived factor 1, vascular endothelial growth factor (VEGF), VEGF-B, VEGF-C, placental growth factor (vascular endothelial growth factor-related protein), transforming growth factor alpha, transforming growth factor beta 1 and its precursors, transforming growth factor beta 2 and its precursors, fibroblast growth factor 1 (acidic), fibroblast growth factor 2 (basic), fibroblast growth factor 5 and its precursors, fibroblast growth factor 6 and its precursors, fibroblast growth factor 7 (keratinocyte growth factor), fibroblast growth factor 8 (androgen-induced), fibroblast growth factor 9 (glia-activating factor), pleiotrophin (heparin binding growth factor 8, neurite growth-promoting factor 1), brain-derived neurotrophic factor, and recombinant glial growth factor 2.

Known proteases include interleukin-1 beta convertase and its precursors, Mch6 and its precursors, Mch2 isoform alpha, Mch4, Cpp32 isoform alpha, Lice2 gamma cysteine protease, Ich-1S, Ich-1L, Ich-2 and its precursors, TY protease, matrix metalloproteinase 1 (interstitial collagenase), matrix metalloproteinase 2 (gelatinase A, 72 kD gelatinase, 72 kD type IV collagenase), matrix metalloproteinase 7 (matrilysin), matrix metalloproteinase 8 (neutrophil collagenase), matrix metalloproteinase 12 (macrophage elastase), matrix metalloproteinase 13 (collagenase 3), metallopeptidase 1, cysteine-rich metalloprotease (disintegrin) and its precursors, subtilisin-like protease Pc8 and its precursors, chymotrypsin, snake venom-like protease, cathepsin 1, cathepsin D (lysosomal aspartyl protease), stromelysin, aminopeptidase N, plasminogen, tissue plasminogen activator, plasminogen activator inhibitor type II, and urokinase-type plasminogen activator.

Known protein kinases include DAP kinase, serine/threonine protein kinases NIK, PK428, Krs-2, SAK, and EMK, interferon-inducible double stranded RNA dependent protein kinase, FAST kinase, AIM1, IPL1-like midbody-associated protein kinase-1, NIMA-like protein kinase 1 (NLK1), the cyclin-dependent kinases (cdk1-10), checkpoint kinase Chk1, Nek3 protein kinase, BMK1 beta kinase, Clk1, Clk2, Clk3, extracellular signal-regulated kinases 1, 3, and 6, cdc28 protein kinase 1, cdc28 protein kinase 2, pLK, Myt1, c-Jun N-terminal kinase 2, Cam kinase 1, the MAP kinases, insulin-stimulated protein kinase 1, beta-adrenergic receptor kinase 2, ribosomal protein S6 kinase, kinase suppressor of ras-1 (KSR1), putative serine/threonine protein kinase Prk, PkB kinase, cAMP-dependent protein kinase, cGMP-dependent protein kinase, type II cGMP-dependent protein kinase, protein kinases Dyrk2, Dyrk3, and Dyrk4, Rho-associated coiled-coil containing protein kinase p160ROCK, protein tyrosine kinase t-Ror1, Ste20-related kinases, cell adhesion kinase beta, protein kinase 3, stress-activated protein kinase 4, protein kinase Zpk, serine kinase hPAK65, dual specificity mitogen-activated protein kinases 1 and 2, casein kinase I gamma 2, p21-activated protein kinase Pak1, lipid-activated protein kinase PRK2, focal adhesion kinase, dual-specificity tyrosine-phosphorylation regulated kinase, myosin light chain kinase, serine kinases SRPK2, TESK1, and VRK2, B lymphocyte serine/threonine protein kinase, stress-activated protein kinases JNK1 and JNK2, phosphorylase kinase, protein tyrosine kinase Tec, Jak2 kinase, protein kinase Ndr, MEK kinase 3, SHB adaptor protein (a Src homology 2 protein), agammaglobulinaemia protein-tyrosine kinase (Atk), protein kinase ATR, guanylate kinase 1, thrombopoeitin receptor and its precursors, DAG kinase epsilon, and kinases encoded by oncogenes or viral oncogenes such as v-fgr (Gardner-Rasheed), v-abl (Abelson murine leukemia viral oncogene homolog 1), v-arg (Abelson murine leukemia viral oncogene homolog, Abelson-related gene), v-fes and v-fps (feline sarcoma viral oncogene and Fujinami avian sarcoma viral oncogene homologs), proto-oncogene c-cot, oncogene pim-1, and oncogene mas1.

It is recognized that the compositions, kits, and methods of the invention will be of particular utility to patients having an enhanced risk of developing prostate cancer and their medical advisors. Patients recognized as having an enhanced risk of developing prostate cancer include, for example, patients having a familial history of prostate cancer, patients identified as having a mutant oncogene (i.e. at least one allele), and patients determined through any other established medical criteria to be at risk for cancer or other malignancy.

The level of expression of a marker gene in normal (i.e. non-cancerous) human prostate tissue can be assessed in a variety of ways. In one embodiment, this normal level of expression is assessed by assessing the level of expression of the marker gene in a portion of prostate cells which appears to be non-cancerous and by comparing this normal level of expression with the level of expression in a portion of the prostate cells which is suspected of being cancerous. For example, the normal level of expression of a marker gene may be assessed using a non-affected portion of the prostate and this normal level of expression may be compared with the level of expression of the same marker gene in an affected portion of the prostate. Alternately, and particularly as further information becomes available as a result of routine performance of the methods described herein, population-average values for normal expression of the marker genes of the invention may be used. In other embodiments, the ‘normal’ level of expression of a marker gene may be determined by assessing expression of the marker gene in a patient sample obtained from a non-cancer-afflicted patient, from a patient sample obtained from a patient before the suspected onset of prostate cancer in the patient, from archived patient samples, and the like.

The invention includes compositions, kits, and methods for assessing the presence of prostate cancer cells in a sample (e.g. an archived tissue sample or a sample obtained from a patient). These compositions, kits, and methods are substantially the same as those described above, except that, where necessary, the compositions, kits, and methods are adapted for use with samples other than patient samples. For example, when the sample to be used is a parafinized, archived human tissue sample, it can be necessary to adjust the ratio of compounds in the compositions of the invention, in the kits of the invention, or the methods used to assess levels of marker gene expression in the sample. Such methods are well known in the art and within the skill of the ordinary artisan.

The invention includes a kit for assessing the presence of prostate cancer cells (e.g. in a sample such as a patient sample). The kit comprises a plurality of reagents, each of which is capable of binding specifically with a nucleic acid or polypeptide corresponding to a marker gene of the invention. Suitable reagents for binding with a polypeptide corresponding to a marker gene of the invention include antibodies, antibody derivatives, antibody fragments, and the like. Suitable reagents for binding with a nucleic acid (e.g. a genomic DNA, an mRNA, a spliced mRNA, a cDNA, or the like) include complementary nucleic acids. For example, the nucleic acid reagents may include oligonucleotides (labeled or non-labeled) fixed to a substrate, labeled oligonucleotides not bound with a substrate, pairs of PCR primers, molecular beacon probes, and the like.

The kit of the invention may optionally comprise additional components useful for performing the methods of the invention. By way of example, the kit may comprise fluids (e.g. SSC buffer) suitable for annealing complementary nucleic acids or for binding an antibody with a protein with which it specifically binds, one or more sample compartments, an instructional material which describes performance of a method of the invention, a sample of normal prostate cells, a sample of prostate cancer cells, and the like.

The invention also includes a method of making an isolated hybridoma which produces an antibody useful for assessing whether a patient is afflicted with prostate cancer. In this method, a protein or protein fragment corresponding to a marker gene of the invention is isolated (e.g. by purification from a cell in which it is expressed or by transcription and translation of a nucleic acid encoding the protein in vivo or in vitro using known methods). A vertebrate, preferably a mammal such as a mouse, rat, rabbit, or sheep, is immunized using the isolated protein or protein fragment. The vertebrate may optionally (and preferably) be immunized at least one additional time with the isolated protein or protein fragment, so that thevertebrate exhibits a robust immune response to the protein or protein fragment. Splenocytes are isolated from the immunized vertebrate and fused with an immortalized cell line to form hybridomas, using any of a variety of methods well known in the art. Hybridomas formed in this manner are then screened using standard methods to identify one or more hybridomas which produce an antibody which specifically binds with the protein or protein fragment. The invention also includes hybridomas made by this method and antibodies made using such hybridomas.

The invention also includes a method of assessing the efficacy of a test compound for inhibiting prostate cancer cells. As described above, increases in the level of expression of the marker genes of the invention correlate with the cancerous state of prostate cells. Although it is recognized that changes in the levels of expression of certain of the marker genes of the invention likely result from the cancerous state of prostate cells, it is likewise recognized that changes in the levels of expression of other of the marker genes of the invention induce, maintain, and promote the cancerous state of those cells. Thus, compounds which inhibit prostate cancer in a patient will cause the level of expression of one or more of the marker genes of the invention to change to a level nearer the normal level of expression for that marker gene (i.e. the level of expression for the marker gene in non-cancerous prostate cells).

This method thus comprises comparing expression of a marker gene in a first prostate cell sample and maintained in the presence of the test compound and expression of the marker gene in a second prostate cell sample and maintained in the absence of the test compound. A significant decreased level of expression of a marker gene listed within Tables 1-3D is an indication that the test compound inhibits prostate cancer. The prostate cell samples may, for example, be aliquots of a single sample of normal prostate cells obtained from a patient, pooled samples of normal prostate cells obtained from a patient, cells of a normal prostate cell line, aliquots of a single sample of prostate cancer cells obtained from a patient, pooled samples of prostate cancer cells obtained from a patient, cells of a prostate cancer cell line, or the like. In one embodiment, the samples are prostate cancer cells obtained from a patient and a plurality of compounds known to be effective for inhibiting various prostate cancers are tested in order to identify the compound which is likely to best inhibit the prostate cancer in the patient.

This method may likewise be used to assess the efficacy of a therapy for inhibiting prostate cancer in a patient. In this method, the level of expression of one or more marker genes of the invention in a pair of samples (one subjected to the therapy, the other not subjected to the therapy) is assessed. As with the method of assessing the efficacy of test compounds, if the therapy induces a significant decrease in the level of expression of a marker gene listed within Tables 1-3D then the therapy is efficacious for inhibiting prostate cancer. As above, if samples from a selected patient are used in this method, then alternative therapies can be assessed in vitro in order to select a therapy most likely to be efficacious for inhibiting prostate cancer in the patient.

As described herein, prostate cancer in patients is associated with an increased level of expression of one or more marker genes listed within Tables 1-3D. While, as discussed above, some of these changes in expression level result from occurrence of the prostate cancer, others of these changes induce, maintain, and promote the cancerous state of prostate cancer cells. Thus, prostate cancer characterized by an increased the level of expression of one or more marker genes listed within Tables 1-3D can be controlled or suppressed by decreasing expression of those marker genes.

Expression of a marker gene listed within Tables 1-3D can be inhibited in a number of ways generally known in the art. For example, an antisense oligonucleotide can be provided to the prostate cancer cells in order to inhibit transcription, translation, or both, of the marker gene(s). Alternately, a polynucleotide encoding an antibody, an antibody derivative, or an antibody fragment, and operably linked with an appropriate promoter/regulator region, can be provided to the cell in order to generate intracellular antibodies which will inhibit the function or activity of the protein corresponding to the marker gene(s). Using the methods described herein, a variety of molecules, particularly including molecules sufficiently small that they are able to cross the cell membrane, can be screened in order to identify molecules which inhibit expression of the marker gene(s). The compound so identified can be provided to the patient in order to inhibit expression of the marker gene(s) in the prostate cancer cells of the patient.

Expression of a marker gene listed within Tables 1-3D can be enhanced in a number of ways generally known in the art. For example, a polynucleotide encoding the marker gene and operably linked with an appropriate promoter/regulator region can be provided to prostate cancer cells of the patient in order to induce enhanced expression of the protein (and mRNA) corresponding to the marker gene therein. Alternatively, if the protein is capable of crossing the cell membrane, inserting itself in the cell membrane, or is normally a secreted protein, then expression of the protein can be enhanced by providing the protein (e.g. directly or by way of the bloodstream or another prostate-associated fluid) to prostate cancer cells in the patient.

As described above, the cancerous state of human prostate cells is correlated with changes in the levels of expression of the marker genes of the invention. Thus, compounds which increase expression of one or more of the marker genes listed within Tables 1-3D can induce prostate cell carcinogenesis. The invention thus includes a method for assessing the human prostate cell carcinogenic potential of a test compound. This method comprises maintaining separate aliquots of human prostate cells in the presence and absence of the test compound. Expression of a marker gene of the invention in each of the aliquots is compared. A significant increase in the level of expression of a marker gene listed within Tables 1-3D in the aliquot maintained in the presence of the test compound (relative to the aliquot maintained in the absence of the test compound) is an indication that the test compound possesses human prostate cell carcinogenic potential. The relative carcinogenic potentials of various test compounds can be assessed by comparing the degree of enhancement or inhibition of the level of expression of the relevant marker genes, by comparing the number of marker genes for which the level of expression is enhanced or inhibited, or by comparing both.

Various aspects of the invention are described in further detail in the following subsections.

I. Isolated Nucleic Acid Molecules

One aspect of the invention pertains to isolated nucleic acid molecules that correspond to a marker gene of the invention, including nucleic acids which encode a polypeptide corresponding to a marker gene of the invention or a portion of such a polypeptide. Isolated nucleic acids of the invention also include nucleic acid molecules sufficient for use as hybridization probes to identify nucleic acid molecules that correspond to a marker gene of the invention, including nucleic acids which encode a polypeptide corresponding to a marker gene of the invention, and fragments of such nucleic acid molecules, e.g., those suitable for use as PCR primers for the amplification or mutation of nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid molecule. Preferably, an “isolated” nucleic acid molecule is free of sequences (preferably protein-encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kB, 4 kB, 3 kB, 2 kB, 1 kB, 0.5 kB or 0.1 kB of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid encoding a protein corresponding to a marker gene listed in one or more of Tables 1-3D, can be isolated using standard molecular biology techniques and the sequence information in the database records described herein. Using all or a portion of such nucleic acid sequences, nucleic acid molecules of the invention can be isolated using standard hybridization and cloning techniques (e.g., as described in Sambrook et al., ed., Molecular Cloning: A Laboratory Manual, 2nd ed., Cold Spring Harbor Laboratory Press, Cold Spring Harbor, N.Y., 1989).

A process for identifying a larger fragment or the full-length coding sequence of a marker gene of the present invention is thus also provided. Any conventional recombinant DNA techniques applicable for isolating polynucleotides may be employed. One such method involves the 5′-RACE-PCR technique, in which the poly-A mRNA that contains the coding sequence of particular interest is first reverse transcribed with a 3′-primer comprising a sequence disclosed herein. The newly synthesized cDNA strand is then tagged with an anchor primer with a known sequence, which preferably contains a convenient cloning restriction site attached at the 5′end. The tagged cDNA is then amplified with the 3′-primer (or a nested primer sharing sequence homology to the internal sequences of the coding region) and the 5′-anchor primer. The amplification may be conducted under conditions of various levels of stringency to optimize the amplification specificity. 5′-RACE-PCR can be readily performed using commercial kits (available from, e.g., BRL Life Technologies Inc., Clotech) according to the manufacturer's instructions.

Isolating the complete coding sequence of a gene can also be carried out in a hybridization assay using a suitable probe. The probe preferably comprises at least 10 nucleotides, and more preferably exhibits sequence homology to the polynucleotides of the marker genes of the present invention. Other high throughput screens for cDNAs, such as those involving gene chip technology, can also be employed in obtaining the complete cDNA sequence.

In addition, databases exist that reduce the complexity of ESTs by assembling contiguous EST sequences into tentative genes. For example, TIGR has assembled human ESTs into a database called THC for tentative human consensus sequences. The THC database allows for a more definitive assignment compared to ESTs alone. Software programs exist (TIGR assembler and TIGEM EST assembly machine and contig assembly program (see Huang, X., 1996, Genomes 33:21-23)) that allow for assembling ESTs into contiguous sequences from any organism.

Alternatively, mRNA from a sample preparation is used to construct cDNA library in the ZAP Express vector following the procedure described in Velculescu et al, 1997, Science 270:484. The ZAP Express cDNA synthesis kit (Stratagene) is used accordingly to the manufacturer's protocol. Plates containing 250 to 2000 plaques are hybridized as described in Rupert et al., 1988, Mol. Cell. Bio. 8:3104 to oligonucleotide probes with the same conditions previously described for standard probes except that the hybridization temperature is reduced to a room temperature. Washes are performed in 6× standard-saline-citrate 0.1% SDS for 30 minutes at room temperature. The probes are labeled with ³²P-ATP trough use of T4 polynucleotide kinase.

A partial cDNA (3′ fragment) can be isolated by 3′ directed PCR reaction. This procedure is a modification of the protocol described in Polyak et al., 1997, Nature 389:300. Briefly, the procedure uses SAGE tags in PCR reaction such that the resultant PCR product contains the SAGE tag of interest as well as additional cDNA, the length of which is defined by the position of the tag with respect to the 3′ end of the cDNA. The cDNA product derived from such a transcript driven PCR reaction can be used for many applications.

RNA from a source to express the cDNA corresponding to a given tag is first converted to double-stranded cDNA using any standard cDNA protocol. Similar conditions used to generate cDNA for SAGE library construction can be employed except that a modified oligo-dT primer is used to derive the first strand synthesis. For example, the oligonucleotide of composition 5′-B-TCC GGC GCG CCG TTT TCC CAG TCA CGA(30)-3′, SEQ ID NO: 16, contains a poly-T stretch at the 3′ end for hybridization and priming from poly-A tails, an M13 priming site for use in subsequent PCR steps, a 5′ Biotin label (B) for capture to strepavidin-coated magnetic beads, and an AscI restriction endonuclease site for releasing the cDNA from the strepavidin-coated magnetic beads. Theoretically, any sufficiently-sized DNA region capable of hybridizing to a PCR primer can be used as well as any other 8 base pair recognizing endonuclease. cDNA constructed utilizing this or similar modified oligo-dT primer is then processed as described in U.S. Pat. No. 5,695,937 up until adapter ligation where only one adapter is ligated to the cDNA pool. After adapter ligation, the cDNA is released from the streptavidin-coated magnetic beads and is then used as a template for cDNA amplification.

Various PCR protocols can be employed using PCR priming sites within the 3′ modified oligo-dT primer and the SAGE tag. The SAGE tag-derived PCR primer employed can be of varying length dictated by 5′ extension of the tag into the adaptor sequence. cDNA products are now available for a variety of applications.

This technique can be further modified by: (1) altering the length and/or content of the modified oligo-dT primer; (2) ligating adaptors other than that previously employed within the SAGE protocol; (3) performing PCR from template retained on the streptavidin-coated magnetic beads; and (4) priming first strand cDNA synthesis with non-oligo-dT based primers.

Gene trapper technology can also be used. The reagents and manufacturer's instructions for this technology are commercially available from Life Technologies, Inc., Gaithsburg, Md. Briefly, a complex population of single-stranded phagemid DNA containing directional cDNA inserts is enriched for the target sequence by hybridization in solution to a biotinylated oligonucleotide probe complementary to the target sequence. The hybrids are captured on streptavidin-coated paramagnetic beads. A magnet retrieves the paramagnetic beads from the solution, leaving nonhybridized single-stranded DNAs behind. Subsequently, the captured single-stranded DNA target is released from the biotinylated oligonucleotide. After release, the cDNA clone is further enriched by using a nonbiotinylated target oligonucleotide to specifically prime conversion of the single-stranded DNA. Following transformation and plating, typically 20% to 100% of the colonies represent the cDNA clone of interest. To identify the desired cDNA clone, the colonies may be screened by colony hybridization using the ³²P-labeled oligonucleotide, or alternatively by DNA sequencing and alignment of all sequences obtained from numerous clones to determine a consensus sequence.

A nucleic acid molecule of the invention can be amplified using cDNA, mRNA, or genomic DNA as a template and appropriate oligonucleotide primers according to standard PCR amplification techniques. The nucleic acid so amplified can be cloned into an appropriate vector and characterized by DNA sequence analysis. Furthermore, oligonucleotides corresponding to all or a portion of a nucleic acid molecule of the invention can be prepared by standard synthetic techniques, e.g., using an automated DNA synthesizer.

In another preferred embodiment, an isolated nucleic acid molecule of the invention comprises a nucleic acid molecule which has a nucleotide sequence complementary to the nucleotide sequence of a nucleic acid corresponding to a marker gene of the invention or to the nucleotide sequence of a nucleic acid encoding a protein which corresponds to a marker gene of the invention. A nucleic acid molecule which is complementary to a given nucleotide sequence is one which is sufficiently complementary to the given nucleotide sequence that it can hybridize to the given nucleotide sequence thereby forming a stable duplex.

Moreover, a nucleic acid molecule of the invention can comprise only a portion of a nucleic acid sequence, wherein the full length nucleic acid sequence comprises a marker gene of the invention or which encodes a polypeptide corresponding to a marker gene of the invention. Such nucleic acids can be used, for example, as a probe or primer. The probe/primer typically is used as one or more substantially purified oligonucleotides. The oligonucleotide typically comprises a region of nucleotide sequence that hybridizes under stringent conditions to at least about 7, preferably about 15, more preferably about 25, 50, 75, 100, 125, 150, 175, 200, 250, 300, 350, or 400 or more consecutive nucleotides of a nucleic acid of the invention.

Probes based on the sequence of a nucleic acid molecule of the invention can be used to detect transcripts or genomic sequences corresponding to one or more marker genes of the invention. The probe comprises a label group attached thereto, e.g., a radioisotope, a fluorescent compound, an enzyme, or an enzyme co-factor. Such probes can be used as part of a diagnostic test kit for identifying cells or tissues which mis-express the protein, such as by measuring levels of a nucleic acid molecule encoding the protein in a sample of cells from a subject, e.g., detecting mRNA levels or determining whether a gene encoding the protein has been mutated or deleted.

The invention further encompasses nucleic acid molecules that differ, due to degeneracy of the genetic code, from the nucleotide sequence of nucleic acids encoding a protein which corresponds to a marker gene of the invention, and thus encode the same protein.

In addition to the nucleotide sequences described in the GenBank and IMAGE Consortium database records described herein, and SEQ ID NOS:1-15 set forth in Table 2, it will be appreciated by those skilled in the art that DNA sequence polymorphisms that lead to changes in the amino acid sequence can exist within a population (e.g., the human population). Such genetic polymorphisms can exist among individuals within a population due to natural allelic variation. An allele is one of a group of genes which occur alternatively at a given genetic locus. In addition, it will be appreciated that DNA polymorphisms that affect RNA expression levels can also exist that may affect the overall expression level of that gene (e.g., by affecting regulation or degradation).

As used herein, the phrase “allelic variant” refers to a nucleotide sequence which occurs at a given locus or to a polypeptide encoded by the nucleotide sequence.

As used herein, the terms “gene” and “recombinant gene” refer to nucleic acid molecules comprising an open reading frame encoding a polypeptide corresponding to a marker gene of the invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

In another embodiment, an isolated nucleic acid molecule of the invention is at least 7, 15, 20, 25, 30, 40, 60, 80, 100, 150, 200, 250, 300, 350, 400, 450, 550, 650, 700, 800, 900, 1000, 1200, 1400, 1600, 1800, 2000, 2200, 2400, 2600, 2800, 3000, 3500, 4000, 4500, or more nucleotides in length and hybridizes under stringent conditions to a nucleic acid corresponding to a marker gene of the invention or to a nucleic acid encoding a protein corresponding to a marker gene of the invention. As used herein, the term “hybridizes under stringent conditions” is intended to describe conditions for hybridization and washing under which nucleotide sequences at least 75% (80%, 85%, preferably 90%) identical to each other typically remain hybridized to each other. Such stringent conditions are known to those skilled in the art and can be found in sections 6.3.1-6.3.6 of Current Protocols in Molecular Biology, John Wiley & Sons, N.Y. (1989). A preferred, non-limiting example of stringent hybridization conditions for annealing two single-stranded DNA each of which is at least about 100 bases in length and/or for annealing a single-stranded DNA and a single-stranded RNA each of which is at least about 100 bases in length, are hybridization in 6×sodium chloride/sodium citrate (SSC) at about 45° C., followed by one or more washes in 0.2×SSC, 0.1% SDS at 50-65° C. Further preferred hybridization conditions are taught in Lockhart, et al., Nature Biotechnology, Volume 14, 1996 August:1675-1680; Breslauer, et al., Proc. Natl. Acad. Sci. USA, Volume 83, 1986 June: 3746-3750; Van Ness, et al., Nucleic Acids Research, Volume 19, No. 19, 1991 September: 5143-5151; McGraw, et al., BioTechniques, Volume 8, No. 6 1990: 674-678; and Milner, et al., Nature Biotechnology, Volume 15, 1997 June: 537-541, all expressly incorporated by reference.

In addition to naturally-occurring allelic variants of a nucleic acid molecule of the invention that can exist in the population, the skilled artisan will further appreciate that sequence changes can be introduced by mutation thereby leading to changes in the amino acid sequence of the encoded protein, without altering the biological activity of the protein encoded thereby. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologs of various species may be non-essential for activity and thus would be likely targets for alteration. Alternatively, amino acid residues that are conserved among the homologs of various species (e.g., murine and human) may be essential for activity and thus would not be likely targets for alteration.

Accordingly, another aspect of the invention pertains to nucleic acid molecules encoding a polypeptide of the invention that contain changes in amino acid residues that are not essential for activity. Such polypeptides differ in amino acid sequence from the naturally-occurring proteins which correspond to the marker genes of the invention, yet retain biological activity. In one embodiment, such a protein has an amino acid sequence that is at least about 40% identical, 50%, 60%, 70%, 80%, 90%, 95%, or 98% identical to the amino acid sequence of one of the proteins which correspond to the marker genes of the invention.

An isolated nucleic acid molecule encoding a variant protein can be created by introducing one or more nucleotide substitutions, additions or deletions into the nucleotide sequence of nucleic acids of the invention, such that one or more amino acid residue substitutions, additions, or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

The present invention encompasses antisense nucleic acid molecules, i.e., molecules which are complementary to a sense nucleic acid of the invention, e.g., complementary to the coding strand of a double-stranded cDNA molecule corresponding to a marker gene of the invention or complementary to an mRNA sequence corresponding to a marker gene of the invention. Accordingly, an antisense nucleic acid of the invention can hydrogen bond to (i.e. anneal with) a sense nucleic acid of the invention. The antisense nucleic acid can be complementary to an entire coding strand, or to only a portion thereof, e.g., all or part of the protein coding region (or open reading frame). An antisense nucleic acid molecule can also be antisense to all or part of a non-coding region of the coding strand of a nucleotide sequence encoding a polypeptide of the invention. The non-coding regions (“5′ and 3′ untranslated regions”) are the 5′ and 3′ sequences which flank the coding region and are not translated into amino acids.

An antisense oligonucleotide can be, for example, about 5, 10, 15, 20, 25, 30, 35, 40, 45, or 50 or more nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been sub-cloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a polypeptide corresponding to a selected marker gene of the invention to thereby inhibit expression of the marker gene, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. Examples of a route of administration of antisense nucleic acid molecules of the invention includes direct injection at a tissue site or infusion of the antisense nucleic acid into a prostate-associated body fluid. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An a-anomeric nucleic acid molecule forms specific double-stranded hybrids with complementary RNA in which, contrary to the usual α-units, the strands run parallel to each other (Gaultier et al., 1987, Nucleic Acids Res. 15:6625-6641). The antisense nucleic acid molecule can also comprise a 2′-o-methylribonucleotide (Inoue et al., 1987, Nucleic Acids Res. 15:6131-6148) or a chimeric RNA-DNA analogue (Inoue et al., 1987, FEBS Lett. 215:327-330).

The invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes as described in Haselhoff and Gerlach, 1988, Nature 334:585-591) can be used to catalytically cleave mRNA transcripts to thereby inhibit translation of the protein encoded by the mRNA. A ribozyme having specificity for a nucleic acid molecule encoding a polypeptide corresponding to a marker gene of the invention can be designed based upon the nucleotide sequence of a cDNA corresponding to the marker gene. For example, a derivative of a Tetrahymena L-19 IVS RNA can be constructed in which the nucleotide sequence of the active site is complementary to the nucleotide sequence to be cleaved (see Cech et al. U.S. Pat. No. 4,987,071; and Cech et al U.S. Pat. No. 5,116,742). Alternatively, an mRNA encoding a polypeptide of the invention can be used to select a catalytic RNA having a specific ribonuclease activity from a pool of RNA molecules (see, e.g., Bartel and Szostak, 1993, Science 261:1411-1418).

The invention also encompasses nucleic acid molecules which form triple helical structures. For example, expression of a polypeptide of the invention can be inhibited by targeting nucleotide sequences complementary to the regulatory region of the gene encoding the polypeptide (e.g., the promoter and/or enhancer) to form triple helical structures that prevent transcription of the gene in target cells. See generally Helene (1991) Anticancer Drug Des. 6(6):569-84; Helene (1992) Ann. N. Y Acad. Sci. 660:27-36; and Maher (1992) Bioassays 14(12):807-15.

In various embodiments, the nucleic acid molecules of the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al., 1996, Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. USA 93:14670-675.

PNAs can be used in therapeutic and diagnostic applications. For example, PNAs can be used as antisense or antigene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S1 nucleases (Hyrup (1996), supra; or as probes or primers for DNA sequence and hybridization (Hyrup, 1996, supra; Perry-O'Keefe et al., 1996, Proc. Natl. Acad. Sci. USA 93:14670-675).

In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which can combine the advantageous properties of PNA and DNA. Such chimeras allow DNA recognition enzymes, e.g., RNASE H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup, 1996, supra). The synthesis of PNA-DNA chimeras can be performed as described in Hyrup (1996), supra, and Finn et al. (1996) Nucleic Acids Res. 24(17):3357-63. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al., 1989, Nucleic Acids Res. 17:5973-88). PNA monomers are then coupled in a step-wise manner to produce a chimeric molecule with a 5′ PNA segment and a 3′ DNA segment (Finn et al., 1996, Nucleic Acids Res. 24(17):3357-63). Alternatively, chimeric molecules can be synthesized with a 5′ DNA segment and a 3′ PNA segment (Peterser et al., 1975, Bioorganic Med. Chem. Lett. 5:1119-11124).

In other embodiments, the oligonucleotide can include other appended groups such as peptides (e.g., for targeting host cell receptors in vivo), or agents facilitating transport across the cell membrane (see, e.g., Letsinger et al., 1989, Proc. Natl. Acad. Sci. USA 86:6553-6556; Lemaitre et al., 1987, Proc. Natl. Acad. Sci. USA 84:648-652; PCT Publication No. WO 88/09810) or the blood-brain barrier (see, e.g., PCT Publication No. WO 89/10134). In addition, oligonucleotides can be modified with hybridization-triggered cleavage agents (see, e.g., Krol et al., 1988, Bio/Techniques 6:958-976) or intercalating agents (see, e.g., Zon, 1988, Pharm. Res. 5:539-549). To this end, the oligonucleotide can be conjugated to another molecule, e.g., a peptide, hybridization triggered cross-linking agent, transport agent, hybridization-triggered cleavage agent, etc.

The invention also includes molecular beacon nucleic acids having at least one region which is complementary to a nucleic acid of the invention, such that the molecular beacon is useful for quantitating the presence of the nucleic acid of the invention in a sample. A “molecular beacon” nucleic acid is a nucleic acid comprising a pair of complementary regions and having a fluorophore and a fluorescent quencher associated therewith. The fluorophore and quencher are associated with different portions of the nucleic acid in such an orientation that when the complementary regions are annealed with one another, fluorescence of the fluorophore is quenched by the quencher. When the complementary regions of the nucleic acid are not annealed with one another, fluorescence of the fluorophore is quenched to a lesser degree. Molecular beacon nucleic acids are described, for example, in U.S. Pat. No. 5,876,930.

II. Isolated Proteins and Antibodies

One aspect of the invention pertains to isolated proteins which correspond to individual marker genes of the invention, and biologically active portions thereof, as well as polypeptide fragments suitable for use as immunogens to raise antibodies directed against a polypeptide corresponding to a marker gene of the invention. In one embodiment, the native polypeptide corresponding to a marker gene can be isolated from cells or tissue sources by an appropriate purification scheme using standard protein purification techniques. In another embodiment, polypeptides corresponding to a marker gene of the invention are produced by recombinant DNA techniques. Alternative to recombinant expression, a polypeptide corresponding to a marker gene of the invention can be synthesized chemically using standard peptide synthesis techniques.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

Biologically active portions of a polypeptide corresponding to a marker gene of the invention include polypeptides comprising amino acid sequences sufficiently identical to or derived from the amino acid sequence of the protein corresponding to the marker gene (e.g., the amino acid sequence listed in the GenBank and IMAGE Consortium database records described herein), which include fewer amino acids than the full length protein, and exhibit at least one activity of the corresponding full-length protein. Typically, biologically active portions comprise a domain or motif with at least one activity of the corresponding protein. A biologically active portion of a protein of the invention can be a polypeptide which is, for example, 10, 25, 50, 100 or more amino acids in length. Moreover, other biologically active portions, in which other regions of the protein are deleted, can be prepared by recombinant techniques and evaluated for one or more of the functional activities of the native form of a polypeptide of the invention.

Preferred polypeptides have the amino acid sequence listed in the one of the GenBank and IMAGE Consortium database records described herein. Other useful proteins are substantially identical (e.g., at least about 40%, preferably 50%, 60%, 70%, 80%, 90%, 95%, or 99%) to one of these sequences and retain the functional activity of the protein of the corresponding naturally-occurring protein yet differ in amino acid sequence due to natural allelic variation or mutagenesis.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment the two sequences are the same length.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLASTand XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules. When utilizing BLAST, Gapped BLAST, and PSI-Blast programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller, (1988) CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad. Sci. USA 85:2444-2448. When using the FASTA algorithm for comparing nucleotide or amino acid sequences, a PAM120 weight residue table can, for example, be used with a k-tuple value of 2.

The percent identity between two sequences can be determined using techniques similar to those described above, with or without allowing gaps. In calculating percent identity, only exact matches are counted.

The invention also provides chimeric or fusion proteins corresponding to a marker gene of the invention. As used herein, a “chimeric protein” or “fusion protein” comprises all or part (preferably a biologically active part) of a polypeptide corresponding to a marker gene of the invention operably linked to a heterologous polypeptide (i.e., a polypeptide other than the polypeptide corresponding to the marker gene). Within the fusion protein, the term “operably linked” is intended to indicate that the polypeptide of the invention and the heterologous polypeptide are fused in-frame to each other. The heterologous polypeptide can be fused to the amino-terminus or the carboxyl-terminus of the polypeptide of the invention.

One useful fusion protein is a GST fusion protein in which a polypeptide corresponding to a marker gene of the invention is fused to the carboxyl terminus of GST sequences. Such fusion proteins can facilitate the purification of a recombinant polypeptide of the invention.

In another embodiment, the fusion protein contains a heterologous signal sequence at its amino terminus. For example, the native signal sequence of a polypeptide corresponding to a marker gene of the invention can be removed and replaced with a signal sequence from another protein. For example, the gp67 secretory sequence of the baculovirus envelope protein can be used as a heterologous signal sequence (Ausubel et al., ed., Current Protocols in Molecular Biology, John Wiley & Sons, NY, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

In yet another embodiment, the fusion protein is an immunoglobulin fusion protein in which all or part of a polypeptide corresponding to a marker gene of the invention is fused to sequences derived from a member of the immunoglobulin protein family. The immunoglobulin fusion proteins of the invention can be incorporated into pharmaceutical compositions and administered to a subject to inhibit an interaction between a ligand (soluble or membrane-bound) and a protein on the surface of a cell (receptor), to thereby suppress signal transduction in vivo. The immunoglobulin fusion protein can be used to affect the bioavailability of a cognate ligand of a polypeptide of the invention. Inhibition of ligand/receptor interaction can be useful therapeutically, both for treating proliferative and differentiative disorders and for modulating (e.g. promoting or inhibiting) cell survival. Moreover, the immunoglobulin fusion proteins of the invention can be used as immunogens to produce antibodies directed against a polypeptide of the invention in a subject, to purify ligands and in screening assays to identify molecules which inhibit the interaction of receptors with ligands.

Chimeric and fusion proteins of the invention can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, e.g., Ausubel et al., supra). Moreover, many expression vectors are commercially available that already encode a fusion moiety (e.g., a GST polypeptide). A nucleic acid encoding a polypeptide of the invention can be cloned into such an expression vector such that the fusion moiety is linked in-frame to the polypeptide of the invention.

A signal sequence can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to polypeptides from which the signal sequence has been proteolytically cleaved (i.e., the cleavage products). In one embodiment, a nucleic acid sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain.

The present invention also pertains to variants of the polypeptides corresponding to individual marker genes of the invention. Such variants have an altered amino acid sequence which can function as either agonists (mimetics) or as antagonists. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a protein of the invention which function as either agonists (mimetics) or as antagonists can be identified by screening combinatorial libraries of mutants, e.g., truncation mutants, of the protein of the invention for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of the polypeptides of the invention from a degenerate oligonucleotide sequence. Methods for synthesizing degenerate oligonucleotides are known in the art (see, e.g., Narang, 1983, Tetrahedron 39:3; Itakura et al., 1984, Annu. Rev. Biochem. 53:323; Itakura et al., 1984, Science 198:1056; Ike et al., 1983 Nucleic Acid Res. 11:477).

In addition, libraries of fragments of the coding sequence of a polypeptide corresponding to a marker gene of the invention can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes amino terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the invention (Arkin and Yourvan, 1992, Proc. Natl. Acad. Sci. USA 89:7811-7815; Delgrave et al., 1993, Protein Engineering 6(3):327-331).

An isolated polypeptide corresponding to a marker gene of the invention, or a fragment thereof, can be used as an immunogen to generate antibodies using standard techniques for polyclonal and monoclonal antibody preparation. The full-length polypeptide or protein can be used or, alternatively, the invention provides antigenic peptide fragments for use as immunogens. The antigenic peptide of a protein of the invention comprises at least 8 (preferably 10, 15, 20, or 30 or more) amino acid residues of the amino acid sequence of one of the polypeptides of the invention, and encompasses an epitope of the protein such that an antibody raised against the peptide forms a specific immune complex with a marker gene of the invention to which the protein corresponds. Preferred epitopes encompassed by the antigenic peptide are regions that are located on the surface of the protein, e.g., hydrophilic regions. Hydrophobicity sequence analysis, hydrophilicity sequence analysis, or similar analyses can be used to identify hydrophilic regions.

An immunogen typically is used to prepare antibodies by immunizing a suitable (i.e. immunocompetent) subject such as a rabbit, goat, mouse, or other mammal or vertebrate. An appropriate immunogenic preparation can contain, for example, recombinantly-expressed or chemically-synthesized polypeptide. The preparation can further include an adjuvant, such as Freund's complete or incomplete adjuvant, or a similar immunostimulatory agent.

Accordingly, another aspect of the invention pertains to antibodies directed against a polypeptide of the invention. The terms “antibody” and “antibody substance” as used interchangeably herein refer to immunoglobulin molecules and immunologically active portions of immunoglobulin molecules, i.e., molecules that contain an antigen binding site which specifically binds an antigen, such as a polypeptide of the invention, e.g., an epitope of a polypeptide of the invention. A molecule which specifically binds to a given polypeptide of the invention is a molecule which binds the polypeptide, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies. The term “monoclonal antibody” or “monoclonal antibody composition”, as used herein, refers to a population of antibody molecules that contain only one species of an antigen binding site capable of immunoreacting with a particular epitope.

Polyclonal antibodies can be prepared as described above by immunizing a suitable subject with a polypeptide of the invention as an immunogen. Preferred polyclonal antibody compositions are ones that have been selected for antibodies directed against a polypeptide or polypeptides of the invention. Particularly preferred polyclonal antibody preparations are ones that contain only antibodies directed against a polypeptide or polypeptides of the invention. Particularly preferred immunogen compositions are those that contain no other human proteins such as, for example, immunogen compositions made using a non-human host cell for recombinant expression of a polypeptide of the invention. In such a manner, the only human epitope or epitopes recognized by the resulting antibody compositions raised against this immunogen will be present as part of a polypeptide or polypeptides of the invention.

The antibody titer in the immunized subject can be monitored over time by standard techniques, such as with an enzyme linked immunosorbent assay (ELISA) using immobilized polypeptide. If desired, the antibody molecules can be harvested or isolated from the subject (e.g., from the blood or serum of the subject) and further purified by well-known techniques, such as protein A chromatography to obtain the IgG fraction. Alternatively, antibodies specific for a protein or polypeptide of the invention can be selected or (e.g., partially purified) or purified by, e.g., affinity chromatography. For example, a recombinantly expressed and purified (or partially purified) protein of the invention is produced as described herein, and covalently or non-covalently coupled to a solid support such as, for example, a chromatography column. The column can then be used to affinity purify antibodies specific for the proteins of the invention from a sample containing antibodies directed against a large number of different epitopes, thereby generating a substantially purified antibody composition, i.e., one that is substantially free of contaminating antibodies. By a substantially purified antibody composition is meant, in this context, that the antibody sample contains at most only 30% (by dry weight) of contaminating antibodies directed against epitopes other than those of the desired protein or polypeptide of the invention, and preferably at most 20%, yet more preferably at most 10%, and most preferably at most 5% (by dry weight) of the sample is contaminating antibodies. A purified antibody composition means that at least 99% of the antibodies in the composition are directed against the desired protein or polypeptide of the invention.

At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (see Kozbor et al., 1983, Immunol. Today 4:72), the EBV-hybridoma technique (see Cole et al., pp. 77-96 In Monoclonal Antibodies and Cancer Therapy, Alan R. Liss, Inc., 1985) or trioma techniques. The technology for producing hybridomas is well known (see generally Current Protocols in Immunology, Coligan et al. ed., John Wiley & Sons, New York, 1994). Hybridoma cells producing a monoclonal antibody of the invention are detected by screening the hybridoma culture supernatants for antibodies that bind the polypeptide of interest, e.g., using a standard ELISA assay.

Alternative to preparing monoclonal antibody-secreting hybridomas, a monoclonal antibody directed against a polypeptide of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant antibodies, such as chimeric and humanized monoclonal antibodies, comprising both human and non-human portions, which can be made using standard recombinant DNA techniques, are within the scope of the invention. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety.) Humanized antibodies are antibody molecules from non-human species having one or more complementarily determining regions (CDRs) from the non-human species and a framework region from a human immunoglobulin molecule. (See, e.g., Queen, U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

Antibodies of the invention may be used as therapeutic agents in treating cancers. In a preferred embodiment, completely human antibodies of the invention are used for therapeutic treatment of human cancer patients, particularly those having an ovarian cancer. Such antibodies can be produced, for example, using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide corresponding to a marker gene of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995) Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a murine antibody, is used to guide the selection of a completely human antibody recognizing the same epitope (Jespers et al., 1994, Bio/technology 12:899-903).

An antibody directed against a polypeptide corresponding to a marker gene of the invention (e.g., a monoclonal antibody) can be used to isolate the polypeptide by standard techniques, such as affinity chromatography or immunoprecipitation. Moreover, such an antibody can be used to detect the marker gene (e.g., in a cellular lysate or cell supernatant) in order to evaluate the level and pattern of expression of the marker gene. The antibodies can also be used diagnostically to monitor protein levels in tissues or body fluids as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Further, an antibody (or fragment thereof) can be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, .alpha.-interferon, .beta.-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

Alternatively, an antibody can be conjugated to a second antibody to form an antibody heteroconjugate as described by Segal in U.S. Pat. No. 4,676,980.

Accordingly, in one aspect, the invention provides substantially purified antibodies or fragments thereof, and non-human antibodies or fragments thereof, which antibodies or fragments specifically bind to a polypeptide comprising an amino acid sequence selected from the group consisting of the amino acid sequences of the present invention, an amino acid sequence encoded by the cDNA of the present invention, a fragment of at least 15 amino acid residues of an amino acid sequence of the present invention, an amino acid sequence which is at least 95% identical to the amino acid sequence of the present invention (wherein the-percent identity is determined using the ALIGN program of the GCG software package with a PAM120 weight residue table, a gap length penalty of 12, and a gap penalty of 4) and an amino acid sequence which is encoded by a nucleic acid molecule which hybridizes to a nucleic acid molecule consisting of the nucleic acid molecules of the present invention, or a complement thereof, under conditions of hybridization of 6×SSC at 45° C. and washing in 0.2×SSC, 0.1% SDS at 65° C. In various embodiments, the substantially purified antibodies of the invention, or fragments thereof, can be human, non-human, chimeric and/or humanized antibodies.

In another aspect, the invention provides non-human antibodies or fragments thereof, which antibodies or fragments specifically bind to a polypeptide comprising an amino acid sequence selected from the group consisting of: the amino acid sequence of the present invention, an amino acid sequence encoded by the cDNA of the present invention, a fragment of at least 15 amino acid residues of the amino acid sequence of the present invention, an amino acid sequence which is at least 95% identical to the amino acid sequence of the present invention (wherein the percent identity is determined using the ALIGN program of the GCG software package with a PAM120 weight residue table, a gap length penalty of 12, and a gap penalty of 4) and an amino acid sequence which is encoded by a nucleic acid molecule which hybridizes to a nucleic acid molecule consisting of the nucleic acid molecules of the present invention, or a complement thereof, under conditions of hybridization of 6×SSC at 45° C. and washing in 0.2×SSC, 0.1% SDS at 65° C. Such non-human antibodies can be goat, mouse, sheep, horse, chicken, rabbit, or rat antibodies. Alternatively, the non-human antibodies of the invention can be chimeric and/or humanized antibodies. In addition, the non-human antibodies of the invention can be polyclonal antibodies or monoclonal antibodies.

In still a further aspect, the invention provides monoclonal antibodies or fragments thereof, which antibodies or fragments specifically bind to a polypeptide comprising an amino acid sequence selected from the group consisting of the amino acid sequences of the present invention, an amino acid sequence encoded by the cDNA of the present invention, a fragment of at least 15 amino acid residues of an amino acid sequence of the present invention, an amino acid sequence which is at least 95% identical to an amino acid sequence of the present invention (wherein the percent identity is determined using the ALIGN program of the GCG software package with a PAM120 weight residue table, a gap length penalty of 12, and a gap penalty of 4) and an amino acid sequence which is encoded by a nucleic acid molecule which hybridizes to a nucleic acid molecule consisting of the nucleic acid molecules of the present invention, or a complement thereof, under conditions of hybridization of 6×SSC at 45° C. and washing in 0.2×SSC, 0.1% SDS at 65° C. The monoclonal antibodies can be human, humanized, chimeric and/or non-human antibodies.

The substantially purified antibodies or fragments thereof may specifically bind to a signal peptide, a secreted sequence, an extracellular domain, a transmembrane or a cytoplasmic domain or cytoplasmic membrane of a polypeptide of the invention. In a particularly preferred embodiment, the substantially purified antibodies or fragments thereof, the non-human antibodies or fragments thereof, and/or the monoclonal antibodies or fragments thereof, of the invention specifically bind to a secreted sequence or an extracellular domain of the amino acid sequences of the present invention.

Any of the antibodies of the invention can be conjugated to a therapeutic moiety or to a detectable substance. Non-limiting examples of detectable substances that can be conjugated to the antibodies of the invention are an enzyme, a prosthetic group, a fluorescent material, a luminescent material, a bioluminescent material, and a radioactive material.

The invention also provides a kit containing an antibody of the invention conjugated to a detectable substance, and instructions for use. Still another aspect of the invention is a pharmaceutical composition comprising an antibody of the invention and a pharmaceutically acceptable carrier. In preferred embodiments, the pharmaceutical composition contains an antibody of the invention, a therapeutic moiety, and a pharmaceutically acceptable carrier.

Still another aspect of the invention is a method of making an antibody that specifically recognizes a polypeptide of the present invention, the method comprising immunizing a mammal with a polypeptide. The polypeptide used as an immungen comprises an amino acid sequence selected from the group consisting of the amino acid sequence of the present invention, an amino acid sequence encoded by the cDNA of the nucleic acid molecules of the present invention, a fragment of at least 15 amino acid residues of the amino acid sequence of the present invention, an amino acid sequence which is at least 95% identical to the amino acid sequence of the present invention (wherein the percent identity is determined using the ALIGN program of the GCG software package with a PAM120 weight residue table, a gap length penalty of 12, and a gap penalty of 4) and an amino acid sequence which is encoded by a nucleic acid molecule which hybridizes to a nucleic acid molecule consisting of the nucleic acid molecules of the present invention, or a complement thereof, under conditions of hybridization of 6×SSC at 45° C. and washing in 0.2×SSC, 0.1% SDS at 65° C.

After immunization, a sample is collected from the mammal that contains an antibody that specifically recognizes the polypeptide. Preferably, the polypeptide is recombinantly produced using a non-human host cell. Optionally, the antibodies can be further purified from the sample using techniques well known to those of skill in the art. The method can further comprise producing a monoclonal antibody-producing cell from the cells of the mammal. Optionally, antibodies are collected from the antibody-producing cell.

III. Recombinant Expression Vectors and Host Cells

Another aspect of the invention pertains to vectors, preferably expression vectors, containing a nucleic acid encoding a polypeptide corresponding to a marker gene of the invention (or a portion of such a polypeptide). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, namely expression vectors, are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Methods in Enzymology: Gene Expression Technology vol. 185, Academic Press, San Diego, Calif. (1991). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein.

The recombinant expression vectors of the invention can be designed for expression of a polypeptide corresponding to a marker gene of the invention in prokaryotic (e.g., E. coli) or eukaryotic cells (e.g., insect cells {using baculovirus expression vectors}, yeast cells or mammalian cells). Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988, Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., 1988, Gene 69:301-315) and pET ld (Studier et al., p. 60-89, In Gene Expression Technology: Methods in Enzymology vol. 185, Academic Press, San Diego, Calif., 1991). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a co-expressed viral RNA polymerase (T7 gn1). This viral polymerase is supplied by host strains BL21 (DE3) or HMS 174(DE3) from a resident prophage harboring a T7 gn1 gene under the transcriptional control of the lactn 5 promoter.

One strategy to maximize recombinant protein expression in E. coli is to express the protein in a host bacteria with an impaired capacity to proteolytically cleave the recombinant protein (Gottesman, p. 119-128, In Gene Expression Technology: Methods in Enzymology vol. 185, Academic Press, San Diego, Calif., 1990. Another strategy is to alter the nucleic acid sequence of the nucleic acid to be inserted into an expression vector so that the individual codons for each amino acid are those preferentially utilized in E. coli (Wada et al., 1992, Nucleic Acids Res. 20:2111-2118). Such alteration of nucleic acid sequences of the invention can be carried out by standard DNA synthesis techniques.

In another embodiment, the expression vector is a yeast expression vector. Examples of vectors for expression in yeast S. cerevisiae include pYepSec1 (Baldari et al., 1987, EMBO J. 6:229-234), pMFa (Kuijan and Herskowitz, 1982, Cell 30:933-943), pJRY88 (Schultz et al., 1987, Gene 54:113-123), pYES2 (Invitrogen Corporation, San Diego, Calif.), and pPicZ (Invitrogen Corp, San Diego, Calif.).

Alternatively, the expression vector is a baculovirus expression vector. Baculovirus vectors available for expression of proteins in cultured insect cells (e.g., Sf 9 cells) include the pAc series (Smith et al., 1983, Mol. Cell Biol. 3:2156-2165) and the pVL series (Lucklow and Summers, 1989, Virology 170:31-39).

In yet another embodiment, a nucleic acid of the invention is expressed in mammalian cells using a mammalian expression vector. Examples of mammalian expression vectors include pCDM8 (Seed, 1987, Nature 329:840) and pMT2NOPC (Kaufman et al., 1987, EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al., supra.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al., 1987, Genes Dev. 1:268-277), lymphoid-specific promoters (Calame and Eaton, 1988, Adv. Immunol. 43:235-275), in particular promoters of T cell receptors (Winoto and Baltimore, 1989, EMBO J. 8:729-733) and immunoglobulins (Baneiji et al., 1983, Cell 33:729-740; Queen and Baltimore, 1983, Cell 33:741-748), neuron-specific promoters (e.g., the neurofilament promoter; Byrne and Ruddle, 1989, Proc. Natl. Acad. Sci. USA 86:5473-5477), pancreas-specific promoters (Edlund et al., 1985, Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss, 1990, Science 249:374-379) and the α-fetoprotein promoter (Camper and Tilghman, 1989, Genes Dev. 3:537-546).

The invention further provides a recombinant expression vector comprising a DNA molecule of the invention cloned into the expression vector in an antisense orientation. That is, the DNA molecule is operably linked to a regulatory sequence in a manner which allows for expression (by transcription of the DNA molecule) of an RNA molecule which is antisense to the mRNA encoding a polypeptide of the invention. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue-specific or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al., 1986, Trends in Genetics, Vol. 1(1).

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic (e.g., E. coli) or eukaryotic cell (e.g., insect cells, yeast or mammalian cells).

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker gene (e.g., for resistance to antibiotics) is generally introduced into the host cells along with the gene of interest. Preferred selectable marker genes include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce a polypeptide corresponding to a marker gene of the invention. Accordingly, the invention further provides methods for producing a polypeptide corresponding to a marker gene of the invention using the host cells of the invention. In one embodiment, the method comprises culturing the host cell of invention (into which a recombinant expression vector encoding a polypeptide of the invention has been introduced) in a suitable medium such that the marker gene is produced. In another embodiment, the method further comprises isolating the marker gene polypeptide from the medium or the host cell.

The host cells of the invention can also be used to produce nonhuman transgenic animals. For example, in one embodiment, a host cell of the invention is a fertilized oocyte or an embryonic stem cell into which a sequences encoding a polypeptide corresponding to a marker gene of the invention have been introduced. Such host cells can then be used to create non-human transgenic animals in which exogenous sequences encoding a marker gene protein of the invention have been introduced into their genome or homologous recombinant animals in which endogenous gene(s) encoding a polypeptide corresponding to a marker gene of the invention sequences have been altered. Such animals are useful for studying the function and/or activity of the polypeptide corresponding to the marker gene and for identifying and/or evaluating modulators of polypeptide activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, an “homologous recombinant animal” is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

A transgenic animal of the invention can be created by introducing a nucleic acid encoding a polypeptide corresponding to a marker gene of the invention into the male pronuclei of a fertilized oocyte, e.g., by microinjection, retroviral infection, and allowing the oocyte to develop in a pseudopregnant female foster animal. Intronic sequences and polyadenylation signals can also be included in the transgene to increase the efficiency of expression of the transgene. A tissue-specific regulatory sequence(s) can be operably linked to the transgene to direct expression of the polypeptide of the invention to particular cells. Methods for generating transgenic animals via embryo manipulation and microinjection, particularly animals such as mice, have become conventional in the art and are described, for example, in U.S. Pat. Nos. 4,736,866 and 4,870,009, U.S. Pat. No. 4,873,191 and in Hogan, Manipulating the Mouse Embryo, Cold Spring Harbor Laboratory Press, Cold Spring Harbor, N.Y., 1986. Similar methods are used for production of other transgenic animals. A transgenic founder animal can be identified based upon the presence of the transgene in its genome and/or expression of mRNA encoding the transgene in tissues or cells of the animals. A transgenic founder animal can then be used to breed additional animals carrying the transgene. Moreover, transgenic animals carrying the transgene can further be bred to other transgenic animals carrying other transgenes.

To create an homologous recombinant animal, a vector is prepared which contains at least a portion of a gene encoding a polypeptide corresponding to a marker gene of the invention into which a deletion, addition or substitution has been introduced to thereby alter, e.g., functionally disrupt, the gene. In a preferred embodiment, the vector is designed such that, upon homologous recombination, the endogenous gene is functionally disrupted (i.e., no longer encodes a functional protein; also referred to as a “knock out” vector). Alternatively, the vector can be designed such that, upon homologous recombination, the endogenous gene is mutated or otherwise altered but still encodes functional protein (e.g., the upstream regulatory region can be altered to thereby alter the expression of the endogenous protein). In the homologous recombination vector, the altered portion of the gene is flanked at its 5′ and 3′ ends by additional nucleic acid of the gene to allow for homologous recombination to occur between the exogenous gene carried by the vector and an endogenous gene in an embryonic stem cell. The additional flanking nucleic acid sequences are of sufficient length for successful homologous recombination with the endogenous gene. Typically, several kilobases of flanking DNA (both at the 5′ and 3′ ends) are included in the vector (see, e.g., Thomas and Capecchi, 1987, Cell 51:503 for a description of homologous recombination vectors). The vector is introduced into an embryonic stem cell line (e.g., by electroporation) and cells in which the introduced gene has homologously recombined with the endogenous gene are selected (see, e.g., Li et al., 1992, Cell 69:915). The selected cells are then injected into a blastocyst of an animal (e.g., a mouse) to form aggregation chimeras (see, e.g., Bradley, Teratocarcinomas and Embryonic Stem Cells: A Practical Approach, Robertson, Ed., IRL, Oxford, 1987, pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Current Opinion in Bio/Technology 2:823-829 and in PCT Publication NOS. WO 90/11354, WO 91/01140, WO 92/0968, and WO 93/04169.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al., 1991, Science 251:1351-1355). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385:810-813 and PCT Publication NOS. WO 97/07668 and WO 97/07669.

IV. Pharmaceutical Compositions

The nucleic acid molecules, polypeptides, and antibodies (also referred to herein as “active compounds”) corresponding to a marker gene of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The invention includes methods for preparing pharmaceutical compositions for modulating the expression or activity of a polypeptide or nucleic acid corresponding to a marker gene of the invention. Such methods comprise formulating a pharmaceutically acceptable carrier with an agent which modulates expression or activity of a polypeptide or nucleic acid corresponding to a marker gene of the invention. Such compositions can further include additional active agents. Thus, the invention further includes methods for preparing a pharmaceutical composition by formulating a pharmaceutically acceptable carrier with an agent which modulates expression or activity of a polypeptide or nucleic acid corresponding to a marker gene of the invention and one or more, additional active compounds.

The invention also provides methods (also referred to herein as “screening assays”) for identifying modulators, i.e., candidate or test compounds or agents (e.g., peptides, peptidomimetics, peptoids, small molecules or other drugs) which (a) bind to the marker gene, or (b) have a modulatory (e.g., stimulatory or inhibitory) effect on the activity of the marker gene or, more specifically, (c) have a modulatory effect on the interactions of the marker gene with one or more of its natural substrates (e.g., peptide, protein, hormone, co-factor, or nucleic acid), or (d) have a modulatory effect on the expression of the marker gene. Such assays typically comprise a reaction between the marker gene and one or more assay components. The other components may be either the test compound itself, or a combination of test compound and a natural binding partner of the marker gene.

The test compounds of the present invention may be obtained from any available source, including systematic libraries of natural and/or synthetic compounds. Test compounds may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckerrnann et al., 1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten, 1992, Biotechniques 13:412-421), or on beads (Lam, 1991, Nature 354:82-84), chips (Fodor, 1993, Nature 364:555-556), bacteria and/or spores, (Ladner, U.S. Pat. No. 5,223,409), plasmids (Cull et al, 1992, Proc Natl Acad Sci USA 89:1865-1869) or on phage (Scott and Smith, 1990, Science 249:386-390; Devlin, 1990, Science 249:404-406; Cwirla et al, 1990, Proc. Natl. Acad. Sci. 87:6378-6382; Felici, 1991, J. Mol. Biol. 222:301-310; Ladner, supra.).

In one embodiment, the invention provides assays for screening candidate or test compounds which are substrates of a marker gene or biologically active portion thereof. In another embodiment, the invention provides assays for screening candidate or test compounds which bind to a marker gene or biologically active portion thereof. Determining the ability of the test compound to directly bind to a marker gene can be accomplished, for example, by coupling the compound with a radioisotope or enzymatic label such that binding of the compound to the marker gene can be determined by detecting the labeled marker gene compound in a complex. For example, compounds (e.g., marker gene substrates) can be labeled with ¹²⁵I, ³⁵S, ¹⁴C, or ³H, either directly or indirectly, and the radioisotope detected by direct counting of radioemission or by scintillation counting. Alternatively, assay components can be enzymatically labeled with, for example, horseradish peroxidase, alkaline phosphatase, or luciferase, and the enzymatic label detected by determination of conversion of an appropriate substrate to product.

In another embodiment, the invention provides assays for screening candidate or test compounds which modulate the activity of a marker gene or a biologically active portion thereof. In all likelihood, the marker gene can, in vivo, interact with one or more molecules, such as but not limited to, peptides, proteins, hormones, cofactors and nucleic acids. For the purposes of this discussion, such cellular and extracellular molecules are referred to herein as “binding partners” or marker gene “substrate”.

One necessary embodiment of the invention in order to facilitate such screening is the use of the marker gene to identify its natural in vivo binding partners. There are many ways to accomplish this which are known to one skilled in the art. One example is the use of the marker gene protein as “bait protein” in a two-hybrid assay or three-hybrid assay (see, e.g., U.S. Pat. No. 5,283,317; Zervos et al, 1993, Cell 72:223-232; Madura et al, 1993, J. Biol. Chem. 268:12046-12054; Bartel et al, 1993, Biotechniques 14:920-924; Iwabuchi et al, 1993 Oncogene 8:1693-1696; Brent WO94/10300) in order to identify other proteins which bind to or interact with the marker gene (binding partners) and, therefore, are possibly involved in the natural function of the marker gene. Such marker gene binding partners are also likely to be involved in the propagation of signals by the marker gene or downstream elements of a marker gene-mediated signaling pathway. Alternatively, such marker gene binding partners may also be found to be inhibitors of the marker gene.

The two-hybrid system is based on the modular nature of most transcription factors, which consist of separable DNA-binding and activation domains. Briefly, the assay utilizes two different DNA constructs. In one construct, the gene that encodes a marker gene protein fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a marker gene-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be readily detected and cell colonies containing the functional transcription factor can be isolated and used to obtain the cloned gene which encodes the protein which interacts with the marker gene protein.

In a further embodiment, assays may be devised through the use of the invention for the purpose of identifying compounds which modulate (e.g., affect either positively or negatively) interactions between a marker gene and its substrates and/or binding partners. Such compounds can include, but are not limited to, molecules such as antibodies, peptides, hormones, oligonucleotides, nucleic acids, and analogs thereof. Such compounds may also be obtained from any available source, including systematic libraries of natural and/or synthetic compounds. The preferred assay components for use in this embodiment is an prostate cancer marker gene identified herein, the known binding partner and/or substrate of same, and the test compound. Test compounds can be supplied from any source.

The basic principle of the assay systems used to identify compounds that interfere with the interaction between the marker gene and its binding partner involves preparing a reaction mixture containing the marker gene and its binding partner under conditions and for a time sufficient to allow the two products to interact and bind, thus forming a complex. In order to test an agent for inhibitory activity, the reaction mixture is prepared in the presence and absence of the test compound. The test compound can be initially included in the reaction mixture, or can be added at a time subsequent to the addition of the marker gene and its binding partner. Control reaction mixtures are incubated without the test compound or with a placebo. The formation of any complexes between the marker gene and its binding partner is then detected. The formation of a complex in the control reaction, but less or no such formation in the reaction mixture containing the test compound, indicates that the compound interferes with the interaction of the marker gene and its binding partner. Conversely, the formation of more complex in the presence of compound than in the control reaction indicates that the compound may enhance interaction of the marker gene and its binding partner.

The assay for compounds that interfere with the interaction of the marker gene with its binding partner may be conducted in a heterogeneous or homogeneous format. Heterogeneous assays involve anchoring either the marker gene or its binding partner onto a solid phase and detecting complexes anchored to the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the marker genes and the binding partners (e.g., by competition) can be identified by conducting the reaction in the presence of the test substance, i.e., by adding the test substance to the reaction mixture prior to or simultaneously with the marker gene and its interactive binding partner. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the marker gene or its binding partner is anchored onto a solid surface or matrix, while the other corresponding non-anchored component may be labeled, either directly or indirectly. In practice, microtitre plates are often utilized for this approach. The anchored species can be immobilized by a number of methods, either non-covalent or covalent, that are typically well known to one who practices the art. Non-covalent attachment can often be accomplished simply by coating the solid surface with a solution of the marker gene or its binding partner and drying. Alternatively, an immobilized antibody specific for the assay component to be anchored can be used for this purpose. Such surfaces can often be prepared in advance and stored.

In related embodiments, a fusion protein can be provided which adds a domain that allows one or both of the assay components to be anchored to a matrix. For example, glutathione-S-transferase/marker gene fusion proteins or glutathione-S-transferase/binding partner can be adsorbed onto glutathione sepharose beads (Sigma Chemical, St. Louis, Mo.) or glutathione derivatized microtiter plates, which are then combined with the test compound or the test compound and either the non-adsorbed marker gene or its binding partner, and the mixture incubated under conditions conducive to complex formation (e.g., physiological conditions). Following incubation, the beads or microtiter plate wells are washed to remove any unbound assay components, the immobilized complex assessed either directly or indirectly, for example, as described above. Alternatively, the complexes can be dissociated from the matrix, and the level of marker gene binding or activity determined using standard techniques.

Other techniques for immobilizing proteins on matrices can also be used in the screening assays of the invention. For example, either a marker gene or a marker gene binding partner can be immobilized utilizing conjugation of biotin and streptavidin. Biotinylated marker gene protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the protein-immobilized surfaces can be prepared in advance and stored.

In order to conduct the assay, the corresponding partner of the immobilized assay component is exposed to the coated surface with or without the test compound. After the reaction is complete, unreacted assay components are removed (e.g., by washing) and any complexes formed will remain immobilized on the solid surface. The detection of complexes anchored on the solid surface can be accomplished in a number of ways. Where the non-immobilized component is pre-labeled, the detection of label immobilized on the surface indicates that complexes were formed. Where the non-immobilized component is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds which modulate (inhibit or enhance) complex formation or which disrupt preformed complexes can be detected.

In an alternate embodiment of the invention, a homogeneous assay may be used. This is typically a reaction, analogous to those mentioned above, which is conducted in a liquid phase in the presence or absence of the test compound. The formed complexes are then separated from unreacted components, and the amount of complex formed is determined. As mentioned for heterogeneous assay systems, the order of addition of reactants to the liquid phase can yield information about which test compounds modulate (inhibit or enhance) complex formation and which disrupt preformed complexes.

In such a homogeneous assay, the reaction products may be separated from unreacted assay components by any of a number of standard techniques, including but not limited to: differential centrifugation, chromatography, electrophoresis and immunoprecipitation. In differential centrifugation, complexes of molecules may be separated from uncomplexed molecules through a series of centrifugal steps, due to the different sedimentation equilibria of complexes based on their different sizes and densities (see, for example, Rivas, G., and Minton, A. P., Trends Biochem Sci August 1993;18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the complex as compared to the uncomplexed molecules may be exploited to differentially separate the complex from the remaining individual reactants, for example through the use of ion-exchange chromatography resins. Such resins and chromatographic techniques are well known to one skilled in the art (see, e.g., Heegaard, 1998, J. Mol. Recognit. 11: 141-148; Hage and Tweed, 1997, J. Chromatogr. B. Biomed. Sci. Appl., 699:499-525). Gel electrophoresis may also be employed to separate complexed molecules from unbound species (see, e.g., Ausubel et al (eds.), In: Current Protocols in Molecular Biology, J. Wiley & Sons, New York. 1999). In this technique, protein or nucleic acid complexes are separated based on size or charge, for example. In order to maintain the binding interaction during the electrophoretic process, nondenaturing gels in the absence of reducing agent are typically preferred, but conditions appropriate to the particular interactants will be well known to one skilled in the art. Immunoprecipitation is another common technique utilized for the isolation of a protein-protein complex from solution (see, e.g., Ausubel et al (eds.), In: Current Protocols in Molecular Biology, J. Wiley & Sons, New York. 1999). In this technique, all proteins binding to an antibody specific to one of the binding molecules are precipitated from solution by conjugating the antibody to a polymer bead that may be readily collected by centrifugation. The bound assay components are released from the beads (through a specific proteolysis event or other technique well known in the art which will not disturb the protein-protein interaction in the complex), and a second immunoprecipitation step is performed, this time utilizing antibodies specific for the correspondingly different interacting assay component. In this manner, only formed complexes should remain attached to the beads. Variations in complex formation in both the presence and the absence of a test compound can be compared, thus offering information about the ability of the compound to modulate interactions between the marker gene and its binding partner.

Also within the scope of the present invention are methods for direct detection of interactions between the marker gene and its natural binding partner and/or a test compound in a homogeneous or heterogeneous assay system without further sample manipulation. For example, the technique of fluorescence energy transfer may be utilized (see, e.g., Lakowicz et al, U.S. Pat. No. 5,631,169; Stavrianopoulos et al, U.S. Pat. No. 4,868,103). Generally, this technique involves the addition of a fluorophore label on a first ‘donor’ molecule (e.g., marker gene or test compound) such that its emitted fluorescent energy will be absorbed by a fluorescent label on a second, ‘acceptor’ molecule (e.g., marker gene or test compound), which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter). A test substance which either enhances or hinders participation of one of the species in the preformed complex will result in the generation of a signal variant to that of background. In this way, test substances that modulate interactions between a marker gene and its binding partner can be identified in controlled assays.

In another embodiment, modulators of marker gene expression are identified in a method wherein a cell is contacted with a candidate compound and the expression of mRNA or protein, corresponding to a marker gene in the cell, is determined. The level of expression of mRNA or protein in the presence of the candidate compound is compared to the level of expression of mRNA or protein in the absence of the candidate compound. The candidate compound can then be identified as a modulator of marker gene expression based on this comparison. For example, when expression of marker gene mRNA or protein is greater (statistically significantly greater) in the presence of the candidate compound than in its absence, the candidate compound is identified as a stimulator of marker gene mRNA or protein expression. Conversely, when expression of marker gene mRNA or protein is less (statistically significantly less) in the presence of the candidate compound than in its absence, the candidate compound is identified as an inhibitor of marker gene mRNA or protein expression. The level of marker gene mRNA or protein expression in the cells can be determined by methods described herein for detecting marker gene mRNA or protein.

In another aspect, the invention pertains to a combination of two or more of the assays described herein. For example, a modulating agent can be identified using a cell-based or a cell free assay, and the ability of the agent to modulate the activity of a marker gene protein can be further confirmed in vivo, e.g., in a whole animal model for cellular transformation and/or tumorigenesis.

This invention further pertains to novel agents identified by the above-described screening assays. Accordingly, it is within the scope of this invention to further use an agent identified as described herein in an appropriate animal model. For example, an agent identified as described herein (e.g., an marker gene modulating agent, an antisense marker gene nucleic acid molecule, an marker gene-specific antibody, or an marker gene-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

It is understood that appropriate doses of small molecule agents and protein or polypeptide agents depends upon a number of factors within the knowledge of the ordinarily skilled physician, veterinarian, or researcher. The dose(s) of these agents will vary, for example, depending upon the identity, size, and condition of the subject or sample being treated, further depending upon the route by which the composition is to be administered, if applicable, and the effect which the practitioner desires the agent to have upon the nucleic acid or polypeptide of the invention. Exemplary doses of a small molecule include milligram or microgram amounts per kilogram of subject or sample weight (e.g. about 1 microgram per kilogram to about 500 milligrams per kilogram, about 100 micrograms per kilogram to about 5 milligrams per kilogram, or about 1 microgram per kilogram to about 50 micrograms per kilogram). Exemplary doses of a protein or polypeptide include gram, milligram or microgram amounts per kilogram of subject or sample weight (e.g. about 1 microgram per kilogram to about 5 grams per kilogram, about 100 micrograms per kilogram to about 500 milligrams per kilogram, or about 1 milligram per kilogram to about 50 milligrams per kilogram). It is furthermore understood that appropriate doses of one of these agents depend upon the potency of the agent with respect to the expression or activity to be modulated. Such appropriate doses can be determined using the assays described herein. When one or more of these agents is to be administered to an animal (e.g. a human) in order to modulate expression or activity of a polypeptide or nucleic acid of the invention, a physician, veterinarian, or researcher can, for example, prescribe a relatively low dose at first, subsequently increasing the dose until an appropriate response is obtained. In addition, it is understood that the specific dose level for any particular animal subject will depend upon a variety of factors including the activity of the specific agent employed, the age, body weight, general health, gender, and diet of the subject, the time of administration, the route of administration, the rate of excretion, any drug combination, and the degree of expression or activity to be modulated.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediamine-tetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium, and then incorporating the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches, and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes having monoclonal antibodies incorporated therein or thereon) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

For antibodies, the preferred dosage is 0.1 mg/kg to 100 mg/kg of body weight (generally 10 mg/kg to 20 mg/kg). If the antibody is to act in the brain, a dosage of 50 mg/kg to 100 mg/kg is usually appropriate. Generally, partially human antibodies and fuilly human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the prostate epithelium). A method for lipidation of antibodies is described by Cruikshank et al. (1997) J. Acquired Immune Deficiency Syndromes and Human Retrovirology 14:193.

The nucleic acid molecules corresponding to a marker gene of the invention can be inserted into vectors and used as gene therapy vectors. Gene therapy vectors can be delivered to a subject by, for example, intravenous injection, local administration (U.S. Pat. No. 5,328,470), or by stereotactic injection (see, e.g., Chen et al., 1994, Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The pharmaceutical compositions can be included in a container, pack, or dispenser together with instructions for administration.

V. Predictive Medicine

The present invention pertains to the field of predictive medicine in which diagnostic assays, prognostic assays, pharmacogenomics, and monitoring clinical trails are used for prognostic (predictive) purposes to thereby treat an individual prophylactically. Accordingly, one aspect of the present invention relates to diagnostic assays for determining the level of expression of polypeptides or nucleic acids corresponding to one or more marker genes of the invention, in order to determine whether an individual is at risk of developing prostate cancer. Such assays can be used for prognostic or predictive purposes to thereby prophylactically treat an individual prior to the onset of the cancer.

Yet another aspect of the invention pertains to monitoring the influence of agents (e.g., drugs or other compounds administered either to inhibit prostate cancer or to treat or prevent any other disorder {i.e. in order to understand any prostate carcinogenic effects that such treatment may have}) on the expression or activity of a marker gene of the invention in clinical trials. These and other agents are described in further detail in the following sections.

A. Diagnostic Assays

An exemplary method for detecting the presence or absence of a polypeptide or nucleic acidecorresponding to a marker gene of the invention in a biological sample involves obtaining a biological sample (e.g. a prostate smear) from a test subject and contacting the biological sample with a compound or an agent capable of detecting the polypeptide or nucleic acid (e.g., mRNA, genomic DNA, or cDNA). The detection methods of the invention can thus be used to detect mRNA, protein, cDNA, or genomic DNA, for example, in a biological sample in vitro as well as in vivo. For example, in vitro techniques for detection of mRNA include Northern hybridizations and in situ hybridizations. In vitro techniques for detection of a polypeptide corresponding to a marker gene of the invention include enzyme linked immunosorbent assays (ELISAs), Western blots, immunoprecipitations, immunohistochemistry and immunofluorescence. In vitro techniques for detection of genomic DNA include Southern hybridizations. Furthermore, in vivo techniques for detection of a polypeptide corresponding to a marker gene of the invention include introducing into a subject a labeled antibody directed against the polypeptide. For example, the antibody can be labeled with a radioactive marker gene whose presence and location in a subject can be detected by standard imaging techniques.

A general principle of such diagnostic and prognostic assays involves preparing a sample or reaction mixture that may contain a marker gene, and a probe, under appropriate conditions and for a time sufficient to allow the marker gene and probe to interact and bind, thus forming a complex that can be removed and/or detected in the reaction mixture. These assays can be conducted in a variety of ways.

For example, one method to conduct such an assay would involve anchoring the marker gene or probe onto a solid phase support, also referred to as a substrate, and detecting target marker gene/probe complexes anchored on the solid phase at the end of the reaction. In one embodiment of such a method, a sample from a subject, which is to be assayed for presence and/or concentration of marker gene, can be anchored onto a carrier or solid phase support. In another embodiment, the reverse situation is possible, in which the probe can be anchored to a solid phase and a sample from a subject can be allowed to react as an unanchored component of the assay.

There are many established methods for anchoring assay components to a solid phase. These include, without limitation, marker gene or probe molecules which are immobilized through conjugation of biotin and streptavidin. Such biotinylated assay components can be prepared from biotin-NHS(N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the surfaces with immobilized assay components can be prepared in advance and stored.

Other suitable carriers or solid phase supports for such assays include any material capable of binding the class of molecule to which the marker gene or probe belongs. Well-known supports or carriers include, but are not limited to, glass, polystyrene, nylon, polypropylene, nylon, polyethylene, dextran, amylases, natural and modified celluloses, polyacrylamides, gabbros, and magnetite.

In order to conduct assays with the above mentioned approaches, the non-immobilized component is added to the solid phase upon which the second component is anchored. After the reaction is complete, uncomplexed components may be removed (e.g., by washing) under conditions such that any complexes formed will remain immobilized upon the solid phase. The detection of marker gene/probe complexes anchored to the solid phase can be accomplished in a number of methods outlined herein.

In a preferred embodiment, the probe, when it is the unanchored assay component, can be labeled for the purpose of detection and readout of the assay, either directly or indirectly, with detectable labels discussed herein and which are well-known to one skilled in the art.

It is also possible to directly detect marker gene/probe complex formation without further manipulation or labeling of either component (marker gene or probe), for example by utilizing the technique of fluorescence energy transfer (see, for example, Lakowicz et al., U.S. Pat. No. 5,631,169; Stavrianopoulos, et al., U.S. Pat. No. 4,868,103). A fluorophore label on the first, ‘donor’ molecule is selected such that, upon excitation with incident light of appropriate wavelength, its emitted fluorescent energy will be absorbed by a fluorescent label on a second ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determination of the ability of a probe to recognize a marker gene can be accomplished without labeling either assay component (probe or marker gene) by utilizing a technology such as real-time Biomolecular Interaction Analysis (BIA) (see, e.g., Sjolander, S. and Urbaniczky, C., 1991, Anal. Chem. 63:2338-2345 and Szabo et al., 1995, Curr. Opin. Struct. Biol. 5:699-705). As used herein, “BIA” or “surface plasmon resonance” is a technology for studying biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

Alternatively, in another embodiment, analogous diagnostic and prognostic assays can be conducted with marker gene and probe as solutes in a liquid phase. In such an assay, the complexed marker gene and probe are separated from uncomplexed components by any of a number of standard techniques, including but not limited to: differential centrifugation, chromatography, electrophoresis and immunoprecipitation. In differential centrifugation, marker gene/probe complexes may be separated from uncomplexed assay components through a series of centrifugal steps, due to the different sedimentation equilibria of complexes based on their different sizes and densities (see, for example, Rivas, G., and Minton, A. P., 1993, Trends Biochem Sci. 18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the marker gene/probe complex as compared to the uncomplexed components may be exploited to differentiate the complex from uncomplexed components, for example through the utilization of ion-exchange chromatography resins. Such resins and chromatographic techniques are well known to one skilled in the art (see, e.g., Heegaard, N. H., 1998, J. Mol. Recognit. Winter 11(1-6):141-8; Hage, D. S., and Tweed, S. A. J. Chromatogr B Biomed Sci Appl Oct. 10, 1997;699(1-2):499-525). Gel electrophoresis may also be employed to separate complexed assay components from unbound components (see, e.g., Ausubel et al., ed., Current Protocols in Molecular Biology, John Wiley & Sons, New York, 1987-1999). In this technique, protein or nucleic acid complexes are separated based on size or charge, for example. In order to maintain the binding interaction during the electrophoretic process, non-denaturing gel matrix materials and conditions in the absence of reducing agent are typically preferred. Appropriate conditions to the particular assay and components thereof will be well known to one skilled in the art.

In a particular embodiment, the level of mRNA corresponding to the marker gene can be determined both by in situ and by in vitro formats in a biological sample using methods known in the art. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA can be utilized for the purification of RNA from prostate cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

The isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding a marker gene of the present invention. Other suitable probes for use in the diagnostic assays of the invention are described herein. Hybridization of an mRNA with the probe indicates that the marker gene in question is being expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods for use in detecting the level of mRNA encoded by the marker genes of the present invention.

An alternative method for determining the level of mRNA corresponding to a marker gene of the present invention in a sample involves the process of nucleic acid amplification, e.g., by rtPCR (the experimental embodiment set forth in Mullis, 1987, U.S. Pat. No. 4,683,202), ligase chain reaction (Barany, 1991, Proc. Natl. Acad. Sci. USA, 88:189-193), self sustained sequence replication (Guatelli et al, 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the prostate cells prior to detection. In such methods, a cell or tissue sample is prepared/processed using known histological methods. The sample is then immobilized on a support, typically a glass slide, and then contacted with a probe that can hybridize to mRNA that encodes the marker gene.

As an alternative to making determinations based on the absolute expression level of the marker gene, determinations may be based on the normalized expression level of the marker gene. Expression levels are normalized by correcting the absolute expression level of a marker gene by comparing its expression to the expression of a gene that is not a marker gene, e.g., a housekeeping gene that is constitutively expressed. Suitable genes for normalization include housekeeping genes such as the actin gene, or epithelial cell-specific genes. This normalization allows the comparison of the expression level in one sample, e.g., a patient sample, to another sample, e.g., a non-prostate cancer sample, or between samples from different sources.

Alternatively, the expression level can be provided as a relative expression level. To determine a relative expression level of a marker gene, the level of expression of the marker gene is determined for 10 or more samples of normal versus cancer cell isolates, preferably 50 or more samples, prior to the determination of the expression level for the sample in question. The mean expression level of each of the genes assayed in the larger number of samples is determined and this is used as a baseline expression level for the marker gene. The expression level of the marker gene determined for the test sample (absolute level of expression) is then divided by the mean expression value obtained for that marker gene. This provides a relative expression level.

Preferably, the samples used in the baseline determination will be from prostate cancer or from non-prostate cancer cells of prostate tissue. The choice of the cell source is dependent on the use of the relative expression level. Using expression found in normal tissues as a mean expression score aids in validating whether the marker gene assayed is prostate specific (versus normal cells). In addition, as more data is accumulated, the mean expression value can be revised, providing improved relative expression values based on accumulated data. Expression data from prostate cells provides a means for grading the severity of the prostate cancer state.

In another embodiment of the present invention, a polypeptide corresponding to a marker gene is detected. A preferred agent for detecting a polypeptide of the invention is an antibody capable of binding to a polypeptide corresponding to a marker gene of the invention, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

Proteins from prostate cells can be isolated using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, N.Y.).

A variety of formats can be employed to determine whether a sample contains a protein that binds to a given antibody. Examples of such formats include, but are not limited to, enzyme immunoassay (EIA), radioimmunoassay (RIA), Western blot analysis, immunohistochemistry and enzyme linked immunoabsorbant assay (ELISA). A skilled artisan can readily adapt known protein/antibody detection methods for use in determining whether prostate cells express a marker gene of the present invention.

In one format, antibodies, or antibody fragments, can be used in methods such as Western blots, immunohistochemistry or immunofluorescence techniques to detect the expressed proteins. In such uses, it is generally preferable to immobilize either the antibody, proteins, or cells containing proteins, on a solid support. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present invention. For example, protein isolated from prostate cells can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means.

The invention also encompasses kits for detecting the presence of a polypeptide or nucleic acid corresponding to a marker gene of the invention in a biological sample (e.g. a prostate sample). Such kits can be used to determine if a subject is suffering from or is at increased risk of developing prostate cancer. For example, the kit can comprise a labeled compound or agent capable of detecting a polypeptide or an mRNA encoding a polypeptide corresponding to a marker gene of the invention in a biological sample and means for determining the amount of the polypeptide or mRNA in the sample (e.g., an antibody which binds the polypeptide or an oligonucleotide probe which binds to DNA or mRNA encoding the polypeptide). Kits can also include instructions for interpreting the results obtained using the kit.

For antibody-based kits, the kit can comprise, for example: (1) a first antibody (e.g., attached to a solid support) which binds to a polypeptide corresponding to a marker gene of the invention; and, optionally, (2) a second, different antibody which binds to either the polypeptide or the first antibody and is conjugated to a detectable label.

For oligonucleotide-based kits, the kit can comprise, for example: (1) an oligonucleotide, e.g., a detectably labeled oligonucleotide, which hybridizes to a nucleic acid sequence encoding a polypeptide corresponding to a marker gene of the invention or (2) a pair of primers useful for amplifying a nucleic acid molecule corresponding to a marker gene of the invention. The kit can also comprise, e.g., a buffering agent, a preservative, or a protein stabilizing agent. The kit can further comprise components necessary for detecting the detectable label (e.g., an enzyme or a substrate). The kit can also contain a control sample or a series of control samples which can be assayed and compared to the test sample. Each component of the kit can be enclosed within an individual container and all of the various containers can be within a single package, along with instructions for interpreting the results of the assays performed using the kit.

B. Pharmacogenomics

Agents or modulators which have a stimulatory or inhibitory effect on expression of a marker gene of the invention can be administered to individuals to treat (prophylactically or therapeutically) prostate cancer in the patient. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the level of expression of a marker gene of the invention in an individual can be determined to thereby select appropriate agent(s) for therapeutic or prophylactic treatment of the individual.

Pharmacogenomics deals with clinically significant variations in the response to drugs due to altered drug disposition and abnormal action in affected persons. See, e.g., Linder (1997) Clin. Chem. 43(2):254-266. In general, two types of pharmacogenetic conditions can be differentiated. Genetic conditions transmitted as a single factor altering the way drugs act on the body are referred to as “altered drug action.” Genetic conditions transmitted as single factors altering the way the body acts on drugs are referred to as “altered drug metabolism”. These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C 19 quite frequently experience exaggerated drug response and side effects when they receive standard doses. If a metabolite is the active therapeutic moiety, a PM will show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the level of expression of a marker gene of the invention in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a modulator of expression of a marker gene of the invention.

This invention also provides a process for preparing a database comprising at least one of the marker genes set forth in Tables 1-3D. For example, the polynucleotide sequences are stored in a digital storage medium such that a data processing system for standardized representation of the genes that identify a prostate cancer cell is compiled. The data processing system is useful to analyze gene expression between two cells by first selecting a cell suspected of being of a neoplastic phenotype or genotype and then isolating polynucleotides from the cell. The isolated polynucleotides are sequenced. The sequences from the sample are compared with the sequence(s) present in the database using homology search techniques. Greater than 90%, more preferably greater than 95% and more preferably, greater than or equal to 97% sequence identity between the test sequence and the polynucleotides of the present invention is a positive indication that the polynucleotide has been isolated from a prostate cancer cell as defined above.

In an alternative embodiment, the polynucleotides of this invention are sequenced and the information regarding sequence and in some embodiments, relative expression, is stored in any functionally relevant program, e.g., in Compare Report using the SAGE software (available though Dr. Ken Kinzler at John Hopkins University). The Compare Report provides a tabulation of the polynucleotide sequences and their abundance for the samples normalized to a defined number of polynucleotides per library (say 25,000). This is then imported into MS-ACCESS either directly or via copying the data into an Excel spreadsheet first and then from there into MS-ACCESS for additional manipulations. Other programs such as SYBASE or Oracle that permit the comparison of polynucleotide numbers could be used as alternatives to MS-ACCESS. Enhancements to the software can be designed to incorporate these additional functions. These functions consist in standard Boolean, algebraic, and text search operations, applied in various combinations to reduce a large input set of polynucleotides to a manageable subset of a polynucleotide of specifically defined interest.

One skilled in the art may create groups containing one or more project(s) by combining the counts of specific polynucleotides within a group (e.g., GroupNormal=Normal1+Normal2, GroupTumor1+Tumor CellLine). Additional characteristic values are also calculated for each tag in the group (e.g., average count, minimum count, maximum count). One skilled in the art may calculate individual tag count ratios between groups, for example the ratio of the average GroupNormal count to the average GroupTumor count for each polynucleotide. A statistical measure of the significance of observed differences in tag counts between groups may be calculated.

C. Monitoring Clinical Trials

Monitoring the influence of agents (e.g., drug compounds) on the level of expression of a marker gene of the invention can be applied not only in basic drug screening, but also in clinical trials. For example, the effectiveness of an agent to affect marker gene expression can be monitored in clinical trials of subjects receiving treatment for prostate cancer. In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate) comprising the steps of (i) obtaining a pre-administration sample from a subject prior to administration of the agent; (ii) detecting the level of expression of one or more selected marker genes of the invention in the pre-administration sample; (iii) obtaining one or more post-administration samples from the subject; (iv) detecting the level of expression of the marker gene(s) in the post-administration samples; (v) comparing the level of expression of the marker gene(s) in the pre-administration sample with the level of expression of the marker gene(s) in the post-administration sample or samples; and (vi) altering the administration of the agent to the subject accordingly. For example, increased administration of the agent can be desirable to increase expression of the marker gene(s) to higher levels than detected, i.e., to increase the effectiveness of the agent. Alternatively, decreased administration of the agent can be desirable to decrease expression of the marker gene(s) to lower levels than detected, i.e., to decrease the effectiveness of the agent.

D. Surrogate Marker genes

The marker genes of the invention may serve as surrogate marker genes for one or more disorders or disease states or for conditions leading up to disease states, and in particular, prostate cancer. As used herein, a “surrogate marker gene” is an objective biochemical marker gene which correlates with the absence or presence of a disease or disorder, or with the progression of a disease or disorder (e.g., with the presence or absence of a tumor). The presence or quantity of such marker genes is independent of the disease., Therefore, these marker genes may serve to indicate whether a particular course of treatment is effective in lessening a disease state or disorder. Surrogate marker genes are of particular use when the presence or extent of a disease state or disorder is difficult to assess through standard methodologies (e.g., early stage tumors), or when an assessment of disease progression is desired before a potentially dangerous clinical endpoint is reached (e.g., an assessment of cardiovascular disease may be made using cholesterol levels as a surrogate marker gene, and an analysis of HIV infection may be made using HIV RNA levels as a surrogate marker gene, well in advance of the undesirable clinical outcomes of myocardial infarction or fully-developed AIDS). Examples of the use of surrogate marker genes in the art include: Koomen et al. (2000) J. Mass. Spectrom. 35: 258-264; and James (1994) AIDS Treatment News Archive 209.

The marker genes of the invention are also useful as pharmacodynamic marker genes. As used herein, a “pharmacodynamic marker gene” is an objective biochemical marker gene which correlates specifically with drug effects. The presence or quantity of a pharmacodynamic marker gene is not related to the disease state or disorder for which the drug is being administered; therefore, the presence or quantity of the marker gene is indicative of the presence or activity of the drug in a subject. For example, a pharmacodynamic marker gene may be indicative of the concentration of the drug in a biological tissue, in that the marker gene is either expressed or transcribed or not expressed or transcribed in that tissue in relationship to the level of the drug. In this fashion, the distribution or uptake of the drug may be monitored by the pharmacodynamic marker gene. Similarly, the presence or quantity of the pharmacodynamic marker gene may be related to the presence or quantity of the metabolic product of a drug, such that the presence or quantity of the marker gene is indicative of the relative breakdown rate of the drug in vivo. Pharmacodynamic marker genes are of particular use in increasing the sensitivity of detection of drug effects, particularly when the drug is administered in low doses. Since even a small amount of a drug may be sufficient to activate multiple rounds of marker gene transcription or expression, the amplified marker gene may be in a quantity which is more readily detectable than the drug itself. Also, the marker gene may be more easily detected due to the nature of the marker gene itself; for example, using the methods described herein, antibodies may be employed in an immune-based detection system for a protein marker gene, or marker gene-specific radiolabeled probes may be used to detect a mRNA marker gene. Furthermore, the use of a pharmacodynamic marker gene may offer mechanism-based prediction of risk due to drug treatment beyond the range of possible direct observations. Examples of the use of pharmacodynamic marker genes in the art include: Matsuda et al. U.S. Pat. No. 6,033,862; Hattis et al. (1991) Env. Health Perspect. 90: 229-238; Schentag (1999) Am. J. Health-Syst. Pharm. 56 Suppl. 3: S21-S24; and Nicolau (1999) Am, J. Health-Syst. Pharm. 56 Suppl. 3: S16-S20.

The marker genes of the invention are also useful as pharmacogenomic marker genes. As used herein, a “pharmacogenomic marker gene” is an objective biochemical marker gene which correlates with a specific clinical drug response or susceptibility in a subject (see, e.g., McLeod et al. (1999) Eur. J. Cancer 35(12): 1650-1652). The presence or quantity of the pharmacogenomic marker gene is related to the predicted response of the subject to a specific drug or class of drugs prior to administration of the drug. By assessing the presence or quantity of one or more pharmacogenomic marker genes in a subject, a drug therapy which is most appropriate for the subject, or which is predicted to have a greater degree of success, may be selected. For example, based on the presence or quantity of RNA or protein for specific tumor marker genes in a subject, a drug or course of treatment miay be selected that is optimized for the treatment of the specific tumor likely to be present in the subject. Similarly, the presence or absence of a specific sequence mutation in marker gene DNA may correlate with drug response. The use of pharmacogenomic marker genes therefore permits the application of the most appropriate treatment for each subject without having to administer the therapy.

VI. Electronic Apparatus Readable Media and Arrays

Electronic apparatus readable media comprising a prostate cancer marker gene of the present invention is also provided. As used herein, “electronic apparatus readable media” refers to any suitable medium for storing, holding or containing data or information that can be read and accessed directly by an electronic apparatus. Such media can include, but are not limited to: magnetic storage media, such as floppy discs, hard disc storage medium, and magnetic tape; optical storage media such as compact disc; electronic storage media such as RAM, ROM, EPROM, EEPROM and the like; general hard disks and hybrids of these categories such as magnetic/optical storage media. The medium is adapted or configured for having recorded thereon a marker gene of the present invention.

As used herein, the term “electronic apparatus” is intended to include any suitable computing or processing apparatus or other device configured or adapted for storing data or information. Examples of electronic apparatus suitable for use with the present invention include stand-alone computing apparatus; networks, including a local area network (LAN), a wide area network (WAN) Internet, Intranet, and Extranet; electronic appliances such as a personal digital assistants (PDAs), cellular phone, pager and the like; and local and distributed processing systems.

As used herein, “recorded” refers to a process for storing or encoding information on the electronic apparatus readable medium. Those skilled in the art can readily adopt any of the presently known methods for recording information on known media to generate manufactures comprising the marker genes of the present invention.

A variety of software programs and formats can be used to store the marker gene information of the present invention on the electronic apparatus readable medium. For example, the nucleic acid sequence corresponding to the marker genes can be represented in a word processing text file, formatted in commercially-available software such as WordPerfect and MicroSoft Word, or represented in the form of an ASCII file, stored in a database application, such as DB2, Sybase, Oracle, or the like, as well as in other forms. Any number of dataprocessor structuring formats (e.g., text file or database) may be employed in order to obtain or create a medium having recorded thereon the marker genes of the present invention.

By providing the marker genes of the invention in readable form, one can routinely access the marker gene sequence information for a variety of purposes. For example, one skilled in the art can use the nucleotide or amino acid sequences of the present invention in readable form to compare a target sequence or target structural motif with the sequence information stored within the data storage means. Search means are used to identify fragments or regions of the sequences of the invention which match a particular target sequence or target motif.

The present invention therefore provides a medium for holding instructions for performing a method for determining whether a subject has prostate cancer or a pre-disposition to prostate cancer, wherein the method comprises the steps of determining the presence or absence of a prostate cancer marker gene and based on the presence or absence of the prostate cancer marker gene, determining whether the subject has prostate cancer or a pre-disposition to prostate cancer and/or recommending a particular treatment for the prostate cancer or pre-prostate cancer condition.

The present invention further provides in an electronic system and/or in a network, a method for determining whether a subject has prostate cancer or a pre-disposition to prostate cancer associated with a prostate cancer marker gene wherein the method comprises the steps of determining the presence or absence of the prostate cancer marker gene, and based on the presence or absence of the prostate cancer marker gene, determining whether the subject has prostate cancer or a pre-disposition to prostate cancer, and/or recommending a particular treatment for the prostate cancer or pre-prostate cancer condition. The method may further comprise the step of receiving phenotypic information associated with the subject and/or acquiring from a network phenotypic information associated with the subject.

The present invention also provides in a network, a method for determining whether a subject has prostate cancer or a pre-disposition to prostate cancer associated with a prostate cancer marker gene, said method comprising the steps of receiving information associated with the prostate cancer marker gene receiving phenotypic information associated with the subject, acquiring information from the network corresponding to the prostate cancer marker gene and/or prostate cancer, and based on one or more of the phenotypic information, the prostate cancer marker gene, and the acquired information, determining whether the subject has prostate cancer or a pre-disposition to prostate cancer. The method may further comprise the step of recommending a particular treatment for the prostate cancer or pre-prostate cancer condition.

The present invention also provides a business method for determining whether a subject has prostate cancer or a pre-disposition to prostate cancer, said method comprising the steps of receiving information associated with the prostate cancer marker gene, receiving phenotypic information associated with the subject, acquiring information from the network corresponding to the prostate cancer marker gene and/or prostate cancer, and based on one or more of the phenotypic information, the prostate cancer marker gene, and the acquired information, determining whether the subject has prostate cancer or a pre-disposition to prostate cancer. The method may further comprise the step of recommending a particular treatment for the prostate cancer or pre-prostate cancer condition.

The invention also includes an array comprising a prostate cancer marker gene of the present invention. The array can be used to assay expression of one or more genes in the array. In one embodiment, the array can be used to assay gene expression in a tissue to ascertain tissue specificity of genes in the array. In this manner, up to about 7600 genes can be simultaneously assayed for expression. This allows a profile to be developed showing a battery of genes specifically expressed in one or more tissues.

In addition to such qualitative determination, the invention allows the quantitation of gene expression. Thus, not only tissue specificity, but also the level of expression of a battery of genes in the tissue is ascertainable. Thus, genes can be grouped on the basis of their tissue expression per se and level of expression in that tissue. This is useful, for example, in ascertaining the relationship of gene expression between or among tissues. Thus, one tissue can be perturbed and the effect on gene expression in a second tissue can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor the time course of expression of one or more genes in the array. This can occur in various biological contexts, as disclosed herein, for example development of prostate cancer, progression of prostate cancer, and processes, such a cellular transformation associated with prostate cancer.

The array is also useful for ascertaining the effect of the expression of a gene on the expression of other genes in the same cell or in different cells. This provides, for example, for a selection of alternate molecular targets for therapeutic intervention if the ultimate or downstream target cannot be regulated.

The array is also useful for ascertaining differential expression patterns of one or more genes in normal and abnormal cells. This provides a battery of genes that could serve as a molecular target for diagnosis or therapeutic intervention.

VII. Experimental Protocol

A. Subtracted Libraries and Transcript Profiling

Subtracted libraries are generated using a PCR based method that allows the isolation of clones expressed at higher levels in one population of mRNA (tester) compared to another population (driver). Both tester and driver mRNA populations are converted into cDNA by reverse transcription, and then PCR amplified using the SMART PCR kit from Clontech. Tester and driver cDNAs are then hybridized using the PCR-Select cDNA subtraction kit from Clontech. This technique results in both subtraction and normalization, which is an equalization of copy number of low-abundance and high-abundance sequences. After generation of the subtractive libraries, a group of 96 or more clones from each library is tested to confirm differential expression by reverse Southern hybridization.

Marker genes 1-11617 (Table 1) were identified through the above-identified subtractive library hybridization techniques. Marker genes 1-1733 were from Library cMhqap; marker genes 1734-3683 were from Library cMhqaq; marker genes 3684-5660 were from Library cMhqar; marker genes 5661-8518 were from Library cMhqao; marker genes 8519-10528 were from Library cMhqaj; marker genes 10529-11617 were from Library cMhqal. The “tester” DNA for Libraries cMhqao, cMhqaj, cMhqal and cMhqap was cDNA obtained from prostate cancer lymph node metastasis RNA. The “tester” DNA for Library cMhqaq was cDNA obtained from prostate cancer liver metastasis RNA. The “tester” DNA for Library cMhqar was cDNA obtained from prostate cancer bone metastasis RNA. The “driver” source for Libraries cMhqao, cMhqap, cMhqaj and cMhqal comprised of cDNAs obtained from clinical good outcome RNA, normal lymph node RNA and tonsil RNA mixed in a 3:1:0.5 ratio respectively. The “driver” source for Library cMhqaq comprised of cDNA obtained from clinical good outcome RNA and normal liver RNA mixed in a 3:1 ratio. The “driver” source for Library cMhqar comprised of cDNA obtained from clinical good outcome RNA. Table 2 includes sequences, identified as SEQ ID NOS: 1-15, for the marker genes of Table 1 which are found in published international applications.

For transcriptional profiling, nylon arrays are prepared by spotting purified PCR product onto a nylon membrane using a robotic gridding system linked to a sample database. Several thousand clones are spotted on each nylon filter. The RNA or DNA is labeled utilizing an in vitro reverse transcription reaction that contains a radiolabeled nucleotide that is incorporated during the reaction. Alternatively, mRNA is converted into cDNA by reverse transcription, and then PCR amplified using the SMART PCR kit from Clontech. Hybridization experiments are carried out by combining labeled RNA or DNA samples with nylon filters in a hybridization chamber. Duplicate, independent hybridization experiments are performed to generate transcriptional profiling data. See, Nature Genetics, 21 (1999). Amplified cDNA is then radiolabelled using random priming with PRIME IT from Stratagene.

Tables 3A-3D list marker genes identified through the above-described transcriptional profiling experiments. Expression of these markers was increased by at least 2-fold or more in the following sequence comparisons:

a) liver metastasis samples, i.e. prostate cancer that had metastasized to the liver, compared to primary prostate tumor samples of good clinical outcome (i.e. tumor samples that were not metastatic) and normal lymph nodes, normal liver and normal prostate samples (Table 3A. “Liver Mets”).

b) bone metastasis samples, i.e. prostate cancer that had metastasized to the bone, compared to primary prostate tumor samples of good clinical outcome and normal lymph nodes, normal liver and normal prostatesamples (Table 3B, “Bone Mets”).

c) metastasized lymph node samples from two different sample sources, i.e. prostate cancer that had metastasized to the lymph nodes, compared to primary prostate tumor samples of good clinical outcome and normal lymph nodes and normal prostate samples (Table 3C, “Nodes 1” and Table 3D, “Nodes 2”).

VIII. Summary Of The Data Provided In The Tables

The description of the fields for Table 1 is listed below:

“Marker Gene” corresponds to the identification number assigned each marker gene of the invention.

“Ace No” corresponds to the accession number assigned to the nucleotide sequence in the relevant database.

“Database” refers to the relevant database where the nucleotide sequence may be found according to its accession number. Those databases which are public include GenBank, dbEST (a division of GenBank), and NUCPATENT (a GENESEQ database, available through Derwent). For examples, see http://www.ncbi.nlm.nih.gov/Entrez/nucleotide.html for GenBank and www.derwent.com for GENESEQ. “GI no” is the GI identification number assigned to the marker gene in the GenBank database (see supra). Nucleic acid sequences of marker genes from Table 1 which are from published international applications are given in Table 2. All referenced database sequences are expressly incorporated herein by reference.

The description of the fields for Tables 3A-3D is listed below:

In Table 3A, “Liver Mets” corresponds to the marker genes of the invention (identified by GI number) that showed increased expression by at least 2-fold or more in liver metastasis samples, i.e. prostate cancer that had metastasized to the liver, compared to primary prostate tumor samples of good clinical outcome (i.e. tumor samples that were not metastatic) and normal lymph nodes, normal liver and normal prostate samples.

In Table 3B, “Bone Mets” corresponds to the marker genes of the invention (identified by GI number) that showed increased expression by at least 2-fold or more in bone metastasis samples, i.e. prostate cancer that had metastasized to the bone, compared to primary prostate tumor samples of good clinical outcome and normal lymph nodes, normal liver and normal prostate samples.

In Tables 3C and 3D, “Nodes 1” and “Nodes 2”, respectively, correspond to the marker genes of the invention (identified by GI number) that showed increased expression by at least 2-fold or more in metastatic lymph node samples from two different sample sources, i.e. prostate cancer that had metastasized to the lymph nodes, compared to primary prostate tumor samples of good clinical outcome, normal lymph node samples and normal prostate samples.

Other Embodiments

Those skilled in the art will recognize, or be able to ascertain using no more than routine experimentation, many equivalents to the specific embodiments of the invention described herein. Such equivalents are intended to be encompassed by the following claims.

All publications including journal references, patents and databases are expressly incorporated by reference.

TABLE 1

| Marker Gene | Acc No | Database | GI no |
|---|---|---|---|
| 1. | A02759 | Genbank | 345130 |
| 2. | A12178 | Genbank | 490111 |
| 3. | A63633 | Genbank | 3717281 |
| 4. | AB000509 | Genbank | 2982670 |
| 5. | AB000878 | Genbank | 3021694 |
| 6. | AB001523 | Genbank | 2244605 |
| 7. | AB002323 | Genbank | 2224590 |
| 8. | AB002346 | Genbank | 6634018 |
| 9. | AB002381 | Genbank | 2224706 |
| 10. | AB002382 | Genbank | 2224708 |
| 11. | AB002387 | Genbank | 2224718 |
| 12. | AB002533 | Genbank | 1944124 |
| 13. | AB003177 | Genbank | 2055255 |
| 14. | AB003476 | Genbank | 2081606 |
| 15. | AB007458 | Genbank | 5006272 |
| 16. | AB007892 | Genbank | 2887434 |
| 17. | AB007959 | Genbank | 3413933 |
| 18. | AB009997 | Genbank | 3219174 |
| 19. | AB011149 | Genbank | 3043677 |
| 20. | AB014519 | Genbank | 3327051 |
| 21. | AB014562 | Genbank | 3327137 |
| 22. | AB015338 | Genbank | 3970863 |
| 23. | AB016043 | Genbank | 4586865 |
| 24. | AB017103 | Genbank | 3493334 |
| 25. | AB018266 | Genbank | 3882166 |
| 26. | AB018276 | Genbank | 3882186 |
| 27. | AB018302 | Genbank | 3882238 |
| 28. | AB018330 | Genbank | 3882294 |
| 29. | AB018342 | Genbank | 3882318 |
| 30. | AB018693 | Genbank | 4587071 |
| 31. | AB020627 | Genbank | 4240125 |
| 32. | AB020657 | Genbank | 4240188 |
| 33. | AB020669 | Genbank | 4240212 |
| 34. | AB020676 | Genbank | 4240226 |
| 35. | AB020685 | Genbank | 4240244 |
| 36. | AB020693 | Genbank | 4240260 |
| 37. | AB020711 | Genbank | 4240296 |
| 38. | AB020724 | Genbank | 4240322 |
| 39. | AB020860 | Genbank | 4003380 |
| 40. | AB020864 | Genbank | 4003384 |
| 41. | AB020865 | Genbank | 4003385 |
| 42. | AB021179 | Genbank | 4062855 |
| 43. | AB021288 | Genbank | 4038732 |
| 44. | AB023049 | Genbank | 5672604 |
| 45. | AB023050 | Genbank | 5672605 |
| 46. | AB023051 | Genbank | 5672606 |
| 47. | AB023056 | Genbank | 5672625 |
| 48. | AB023157 | Genbank | 4589523 |
| 49. | AB023208 | Genbank | 4589625 |
| 50. | AB023230 | Genbank | 4589675 |
| 51. | AB023691 | Genbank | 6519222 |
| 52. | AB024334 | Genbank | 6016837 |
| 53. | AB026898 | Genbank | 4835382 |
| 54. | AB027013 | Genbank | 5931609 |
| 55. | AB028988 | Genbank | 5689466 |
| 56. | AB032160 | Genbank | 7328967 |
| 57. | AB032951 | Genbank | 6329748 |
| 58. | AB033049 | Genbank | 6330616 |
| 59. | AB033082 | Genbank | 6330910 |
| 60. | AB037744 | Genbank | 7243026 |
| 61. | AB037745 | Genbank | 7243028 |
| 62. | AB037752 | Genbank | 7243042 |
| 63. | AB037796 | Genbank | 7243130 |
| 64. | AB037802 | Genbank | 7243142 |
| 65. | AB037807 | Genbank | 7243152 |
| 66. | AB037825 | Genbank | 7243188 |
| 67. | AB037860 | Genbank | 7243275 |
| 68. | AB038161 | Genbank | 10280531 |
| 69. | AB042031 | Genbank | 9711455 |
| 70. | AB045357 | Genbank | 8918412 |
| 71. | AB046767 | Genbank | 10047158 |
| 72. | AB046773 | Genbank | 10047170 |
| 73. | AB046813 | Genbank | 10047260 |
| 74. | AB048955 | Genbank | 10241853 |
| 75. | AC000034 | Genbank | 4582474 |
| 76. | AC000043 | Genbank | 7923869 |
| 77. | AC000047 | Genbank | 5174830 |
| 78. | AC000065 | Genbank | 1669367 |
| 79. | AC000105 | Genbank | 7923862 |
| 80. | AC000117 | Genbank | 1809228 |
| 81. | AC000120 | Genbank | 1809224 |
| 82. | AC000353 | Genbank | 6970735 |
| 83. | AC000378 | Genbank | 2270906 |
| 84. | AC000388 | Genbank | 2160131 |
| 85. | AC000394 | Genbank | 2133911 |
| 86. | AC001228 | Genbank | 1935053 |
| 87. | AC001234 | Genbank | 4028939 |
| 88. | AC002040 | Genbank | 2347081 |
| 89. | AC002044 | Genbank | 2347079 |
| 90. | AC002045 | Genbank | 2951945 |
| 91. | AC002078 | Genbank | 2078458 |
| 92. | AC002109 | Genbank | 2347182 |
| 93. | AC002117 | Genbank | 2281075 |
| 94. | AC002123 | Genbank | 2121321 |
| 95. | AC002124 | Genbank | 2121320 |
| 96. | AC002297 | Genbank | 2182280 |
| 97. | AC002306 | Genbank | 2213634 |
| 98. | AC002326 | Genbank | 2288970 |
| 99. | AC002352 | Genbank | 3858888 |
| 100. | AC002364 | Genbank | 3399662 |
| 101. | AC002381 | Genbank | 2275186 |
| 102. | AC002386 | Genbank | 2275181 |
| 103. | AC002390 | Genbank | 2282011 |
| 104. | AC002395 | Genbank | 3540146 |
| 105. | AC002402 | Genbank | 2642148 |
| 106. | AC002418 | Genbank | 2822137 |
| 107. | AC002433 | Genbank | 2335063 |
| 108. | AC002449 | Genbank | 2337886 |
| 109. | AC002450 | Genbank | 2337884 |
| 110. | AC002463 | Genbank | 2337866 |
| 111. | AC002464 | Genbank | 2337864 |
| 112. | AC002469 | Genbank | 8810486 |
| 113. | AC002481 | Genbank | 2340092 |
| 114. | AC002542 | Genbank | 2393733 |
| 115. | AC002544 | Genbank | 3337382 |
| 116. | AC002546 | Genbank | 2583101 |
| 117. | AC002550 | Genbank | 2570261 |
| 118. | AC002553 | Genbank | 3126783 |
| 119. | AC002558 | Genbank | 2580474 |
| 120. | AC002563 | Genbank | 2439515 |
| 121. | AC002996 | Genbank | 3132461 |
| 122. | AC003009 | Genbank | 2734100 |
| 123. | AC003071 | Genbank | 2588643 |
| 124. | AC003077 | Genbank | 2588634 |
| 125. | AC003080 | Genbank | 2588627 |
| 126. | AC003082 | Genbank | 2588625 |
| 127. | AC003101 | Genbank | 3184508 |
| 128. | AC003111 | Genbank | 2636670 |
| 129. | AC003663 | Genbank | 3097871 |
| 130. | AC003664 | Genbank | 2828774 |
| 131. | AC003985 | Genbank | 2769692 |
| 132. | AC003991 | Genbank | 2772535 |
| 133. | AC003999 | Genbank | 2772566 |
| 134. | AC004001 | Genbank | 2772562 |
| 135. | AC004003 | Genbank | 2772557 |
| 136. | AC004022 | Genbank | 3598727 |
| 137. | AC004052 | Genbank | 3075380 |
| 138. | AC004059 | Genbank | 4263637 |
| 139. | AC004061 | Genbank | 3108028 |
| 140. | AC004062 | Genbank | 3342741 |
| 141. | AC004078 | Genbank | 2822148 |
| 142. | AC004083 | Genbank | 2822159 |
| 143. | AC004084 | Genbank | 2822156 |
| 144. | AC004109 | Genbank | 2828788 |
| 145. | AC004140 | Genbank | 2880082 |
| 146. | AC004148 | Genbank | 3482960 |
| 147. | AC004166 | Genbank | 8887011 |
| 148. | AC004216 | Genbank | 3097816 |
| 149. | AC004227 | Genbank | 2911718 |
| 150. | AC004228 | Genbank | 4263838 |
| 151. | AC004254 | Genbank | 2920818 |
| 152. | AC004382 | Genbank | 3252819 |
| 153. | AC004386 | Genbank | 3046272 |
| 154. | AC004417 | Genbank | 2979583 |
| 155. | AC004453 | Genbank | 2979600 |
| 156. | AC004459 | Genbank | 2979594 |
| 157. | AC004460 | Genbank | 2981263 |
| 158. | AC004462 | Genbank | 7122648 |
| 159. | AC004466 | Genbank | 3617739 |
| 160. | AC004467 | Genbank | 3132844 |
| 161. | AC004518 | Genbank | 3004535 |
| 162. | AC004531 | Genbank | 3337392 |
| 163. | AC004584 | Genbank | 3417305 |
| 164. | AC004585 | Genbank | 3212882 |
| 165. | AC004594 | Genbank | 3063516 |
| 166. | AC004597 | Genbank | 3068558 |
| 167. | AC004610 | Genbank | 3080646 |
| 168. | AC004612 | Genbank | 3080666 |
| 169. | AC004617 | Genbank | 11128429 |
| 170. | AC004620 | Genbank | 3093423 |
| 171. | AC004626 | Genbank | 3337396 |
| 172. | AC004671 | Genbank | 3810574 |
| 173. | AC004686 | Genbank | 3688105 |
| 174. | AC004704 | Genbank | 4417258 |
| 175. | AC004707 | Genbank | 3249127 |
| 176. | AC004739 | Genbank | 3152634 |
| 177. | AC004741 | Genbank | 3152631 |
| 178. | AC004760 | Genbank | 3168626 |
| 179. | AC004774 | Genbank | 3169209 |
| 180. | AC004775 | Genbank | 3169300 |
| 181. | AC004776 | Genbank | 3169299 |
| 182. | AC004782 | Genbank | 3172145 |
| 183. | AC004814 | Genbank | 5708498 |
| 184. | AC004819 | Genbank | 4153878 |
| 185. | AC004837 | Genbank | 3845417 |
| 186. | AC004842 | Genbank | 4753286 |
| 187. | AC004849 | Genbank | 3980553 |
| 188. | AC004855 | Genbank | 3980550 |
| 189. | AC004858 | Genbank | 6624125 |
| 190. | AC004859 | Genbank | 5091656 |
| 191. | AC004876 | Genbank | 4508148 |
| 192. | AC004881 | Genbank | 3900853 |
| 193. | AC004884 | Genbank | 4156189 |
| 194. | AC004894 | Genbank | 3478667 |
| 195. | AC004904 | Genbank | 4156180 |
| 196. | AC004908 | Genbank | 4156179 |
| 197. | AC004909 | Genbank | 5001545 |
| 198. | AC004911 | Genbank | 3478665 |
| 199. | AC004922 | Genbank | 4753276 |
| 200. | AC004928 | Genbank | 4587636 |
| 201. | AC004938 | Genbank | 5103908 |
| 202. | AC004943 | Genbank | 3924671 |
| 203. | AC004955 | Genbank | 4753267 |
| 204. | AC004960 | Genbank | 3845412 |
| 205. | AC004968 | Genbank | 4156160 |
| 206. | AC004973 | Genbank | 3694660 |
| 207. | AC004975 | Genbank | 4753261 |
| 208. | AC004980 | Genbank | 9755474 |
| 209. | AC004984 | Genbank | 3355524 |
| 210. | AC004985 | Genbank | 5708490 |
| 211. | AC004986 | Genbank | 4309812 |
| 212. | AC004990 | Genbank | 3924668 |
| 213. | AC004997 | Genbank | 5441941 |
| 214. | AC004999 | Genbank | 3970963 |
| 215. | AC005011 | Genbank | 4753254 |
| 216. | AC005034 | Genbank | 3947437 |
| 217. | AC005041 | Genbank | 4508118 |
| 218. | AC005043 | Genbank | 4926890 |
| 219. | AC005052 | Genbank | 10122134 |
| 220. | AC005057 | Genbank | 6587915 |
| 221. | AC005065 | Genbank | 4153861 |
| 222. | AC005076 | Genbank | 4508110 |
| 223. | AC005082 | Genbank | 5836159 |
| 224. | AC005086 | Genbank | 5708461 |
| 225. | AC005088 | Genbank | 4753221 |
| 226. | AC005091 | Genbank | 4156134 |
| 227. | AC005094 | Genbank | 3478660 |
| 228. | AC005154 | Genbank | 3242763 |
| 229. | AC005159 | Genbank | 3242756 |
| 230. | AC005179 | Genbank | 3258607 |
| 231. | AC005189 | Genbank | 3264580 |
| 232. | AC005213 | Genbank | 3282167 |
| 233. | AC005229 | Genbank | 4153865 |
| 234. | AC005249 | Genbank | 3287720 |
| 235. | AC005255 | Genbank | 3289998 |
| 236. | AC005300 | Genbank | 6492490 |
| 237. | AC005301 | Genbank | 7107554 |
| 238. | AC005316 | Genbank | 3738342 |
| 239. | AC005318 | Genbank | 3885345 |
| 240. | AC005324 | Genbank | 3366582 |
| 241. | AC005326 | Genbank | 3341714 |
| 242. | AC005328 | Genbank | 3342735 |
| 243. | AC005347 | Genbank | 3366565 |
| 244. | AC005368 | Genbank | 3367506 |
| 245. | AC005369 | Genbank | 3367505 |
| 246. | AC005382 | Genbank | 3386587 |
| 247. | AC005389 | Genbank | 3419836 |
| 248. | AC005412 | Genbank | 11128436 |
| 249. | AC005479 | Genbank | 4508155 |
| 250. | AC005481 | Genbank | 7321905 |
| 251. | AC005484 | Genbank | 5091654 |
| 252. | AC005495 | Genbank | 3810670 |
| 253. | AC005500 | Genbank | 7798766 |
| 254. | AC005520 | Genbank | 5001541 |
| 255. | AC005523 | Genbank | 3451032 |
| 256. | AC005531 | Genbank | 4153875 |
| 257. | AC005536 | Genbank | 4454514 |
| 258. | AC005537 | Genbank | 4753251 |
| 259. | AC005546 | Genbank | 3478635 |
| 260. | AC005548 | Genbank | 3687279 |
| 261. | AC005565 | Genbank | 3493166 |
| 262. | AC005574 | Genbank | 3510227 |
| 263. | AC005576 | Genbank | 3510225 |
| 264. | AC005582 | Genbank | 3510268 |
| 265. | AC005599 | Genbank | 4388706 |
| 266. | AC005610 | Genbank | 3540155 |
| 267. | AC005612 | Genbank | 3540153 |
| 268. | AC005618 | Genbank | 3548785 |
| 269. | AC005630 | Genbank | 4159882 |
| 270. | AC005666 | Genbank | 4049331 |
| 271. | AC005670 | Genbank | 3688099 |
| 272. | AC005682 | Genbank | 5757537 |
| 273. | AC005686 | Genbank | 3608161 |
| 274. | AC005690 | Genbank | 6850335 |
| 275. | AC005694 | Genbank | 4996908 |
| 276. | AC005726 | Genbank | 3810672 |
| 277. | AC005741 | Genbank | 3687209 |
| 278. | AC005763 | Genbank | 3694628 |
| 279. | AC005768 | Genbank | 6598827 |
| 280. | AC005785 | Genbank | 3702290 |
| 281. | AC005799 | Genbank | 3789715 |
| 282. | AC005828 | Genbank | 3789713 |
| 283. | AC005829 | Genbank | 3873300 |
| 284. | AC005837 | Genbank | 3849820 |
| 285. | AC005841 | Genbank | 4731044 |
| 286. | AC005854 | Genbank | 4454568 |
| 287. | AC005866 | Genbank | 4731043 |
| 288. | AC005876 | Genbank | 6249672 |
| 289. | AC005880 | Genbank | 6249667 |
| 290. | AC005886 | Genbank | 6382479 |
| 291. | AC005903 | Genbank | 6579238 |
| 292. | AC005905 | Genbank | 4090182 |
| 293. | AC005908 | Genbank | 4165007 |
| 294. | AC005909 | Genbank | 4165006 |
| 295. | AC005913 | Genbank | 9966229 |
| 296. | AC005939 | Genbank | 3873180 |
| 297. | AC005949 | Genbank | 6862981 |
| 298. | AC005968 | Genbank | 3907448 |
| 299. | AC005969 | Genbank | 4309946 |
| 300. | AC005971 | Genbank | 3900838 |
| 301. | AC005996 | Genbank | 4309892 |
| 302. | AC005999 | Genbank | 10801431 |
| 303. | AC006012 | Genbank | 4309814 |
| 304. | AC006021 | Genbank | 4731073 |
| 305. | AC006029 | Genbank | 4508135 |
| 306. | AC006033 | Genbank | 4309948 |
| 307. | AC006038 | Genbank | 7321960 |
| 308. | AC006050 | Genbank | 4079627 |
| 309. | AC006058 | Genbank | 4204247 |
| 310. | AC006059 | Genbank | 4544348 |
| 311. | AC006063 | Genbank | 4204699 |
| 312. | AC006115 | Genbank | 3962498 |
| 313. | AC006121 | Genbank | 4062176 |
| 314. | AC006128 | Genbank | 3970934 |
| 315. | AC006141 | Genbank | 4580411 |
| 316. | AC006146 | Genbank | 5836157 |
| 317. | AC006151 | Genbank | 6094678 |
| 318. | AC006160 | Genbank | 5701616 |
| 319. | AC006208 | Genbank | 4558540 |
| 320. | AC006211 | Genbank | 4071020 |
| 321. | AC006241 | Genbank | 4160141 |
| 322. | AC006254 | Genbank | 4726099 |
| 323. | AC006256 | Genbank | 4090190 |
| 324. | AC006271 | Genbank | 4092817 |
| 325. | AC006350 | Genbank | 4508140 |
| 326. | AC006355 | Genbank | 4753266 |
| 327. | AC006364 | Genbank | 4753253 |
| 328. | AC006372 | Genbank | 4753237 |
| 329. | AC006373 | Genbank | 5708468 |
| 330. | AC006376 | Genbank | 4508126 |
| 331. | AC006392 | Genbank | 4150933 |
| 332. | AC006396 | Genbank | 4156191 |
| 333. | AC006409 | Genbank | 4454486 |
| 334. | AC006427 | Genbank | 6437518 |
| 335. | AC006453 | Genbank | 4753285 |
| 336. | AC006460 | Genbank | 7243904 |
| 337. | AC006475 | Genbank | 4753283 |
| 338. | AC006480 | Genbank | 6289252 |
| 339. | AC006512 | Genbank | 4926863 |
| 340. | AC006518 | Genbank | 4713939 |
| 341. | AC006529 | Genbank | 4225932 |
| 342. | AC006530 | Genbank | 4680764 |
| 343. | AC006557 | Genbank | 4263667 |
| 344. | AC006977 | Genbank | 5931479 |
| 345. | AC006987 | Genbank | 5123989 |
| 346. | AC006994 | Genbank | 8748916 |
| 347. | AC007006 | Genbank | 4827306 |
| 348. | AC007041 | Genbank | 5708471 |
| 349. | AC007051 | Genbank | 4432877 |
| 350. | AC007057 | Genbank | 4680440 |
| 351. | AC007066 | Genbank | 4508098 |
| 352. | AC007094 | Genbank | 7637802 |
| 353. | AC007114 | Genbank | 4581193 |
| 354. | AC007124 | Genbank | 4454586 |
| 355. | AC007160 | Genbank | 5348408 |
| 356. | AC007172 | Genbank | 4731066 |
| 357. | AC007193 | Genbank | 4558635 |
| 358. | AC007216 | Genbank | 6806842 |
| 359. | AC007226 | Genbank | 6715702 |
| 360. | AC007244 | Genbank | 7705226 |
| 361. | AC007254 | Genbank | 6560919 |
| 362. | AC007271 | Genbank | 7408074 |
| 363. | AC007276 | Genbank | 6587937 |
| 364. | AC007279 | Genbank | 10440749 |
| 365. | AC007283 | Genbank | 5931446 |
| 366. | AC007285 | Genbank | 5931439 |
| 367. | AC007312 | Genbank | 4586080 |
| 368. | AC007314 | Genbank | 4662678 |
| 369. | AC007320 | Genbank | 10801478 |
| 370. | A0007321 | Genbank | 7243928 |
| 371. | AC007324 | Genbank | 7923342 |
| 372. | AC007327 | Genbank | 4587630 |
| 373. | AC007370 | Genbank | 4883671 |
| 374. | AC007371 | Genbank | 5542041 |
| 375. | AC007390 | Genbank | 6358845 |
| 376. | AC007406 | Genbank | 4689442 |
| 377. | AC007428 | Genbank | 9369503 |
| 378. | AC007429 | Genbank | 5069494 |
| 379. | AC007435 | Genbank | 5091640 |
| 380. | AC007465 | Genbank | 10801482 |
| 381. | AC007511 | Genbank | 9369505 |
| 382. | AC007533 | Genbank | 6682595 |
| 383. | AC007563 | Genbank | 6289217 |
| 384. | AC007617 | Genbank | 5230397 |
| 385. | AC007652 | Genbank | 4887764 |
| 386. | AC007664 | Genbank | 5903121 |
| 387. | AC007677 | Genbank | 5931475 |
| 388. | AC007682 | Genbank | 10440752 |
| 389. | AC007684 | Genbank | 5836173 |
| 390. | AC007688 | Genbank | 5815499 |
| 391. | AC007690 | Genbank | 5815494 |
| 392. | AC007744 | Genbank | 7243926 |
| 393. | AC007750 | Genbank | 6094634 |
| 394. | AC007751 | Genbank | 5001551 |
| 395. | AC007773 | Genbank | 5032322 |
| 396. | AC007790 | Genbank | 5042474 |
| 397. | AC007860 | Genbank | 5649179 |
| 398. | AC007911 | Genbank | 6682264 |
| 399. | AC007934 | Genbank | 6721208 |
| 400. | AC007969 | Genbank | 7534288 |
| 401. | AC008010 | Genbank | 6137874 |
| 402. | AC008011 | Genbank | 9558562 |
| 403. | AC008040 | Genbank | 5922025 |
| 404. | AC008045 | Genbank | 6751673 |
| 405. | AC008062 | Genbank | 5931476 |
| 406. | AC008067 | Genbank | 6587932 |
| 407. | AC008069 | Genbank | 6094640 |
| 408. | AC008070 | Genbank | 8748906 |
| 409. | AC008102 | Genbank | 9797815 |
| 410. | AC008109 | Genbank | 6984389 |
| 411. | AC008115 | Genbank | 5801654 |
| 412. | AC008116 | Genbank | 6001959 |
| 413. | AC008122 | Genbank | 5931373 |
| 414. | AC008123 | Genbank | 6006046 |
| 415. | AC008124 | Genbank | 5815491 |
| 416. | AC008149 | Genbank | 6630488 |
| 417. | AC008151 | Genbank | 5629925 |
| 418. | AC008154 | Genbank | 6729061 |
| 419. | AC008164 | Genbank | 7243886 |
| 420. | AC008169 | Genbank | 6587935 |
| 421. | AC008171 | Genbank | 10716651 |
| 422. | AC008243 | Genbank | 8347984 |
| 423. | AC008249 | Genbank | 6137875 |
| 424. | AC008268 | Genbank | 6604540 |
| 425. | AC008269 | Genbank | 10716637 |
| 426. | AC008270 | Genbank | 7243891 |
| 427. | AC008279 | Genbank | 7408141 |
| 428. | AC008394 | Genbank | 6850300 |
| 429. | AC008417 | Genbank | 6730695 |
| 430. | AC008524 | Genbank | 9625319 |
| 431. | AC008534 | Genbank | 9558573 |
| 432. | AC008543 | Genbank | 8886965 |
| 433. | AC008545 | Genbank | 6721145 |
| 434. | AC008551 | Genbank | 9625320 |
| 435. | AC008556 | Genbank | 9558574 |
| 436. | AC008572 | Genbank | 7341444 |
| 437. | AC008649 | Genbank | 9690315 |
| 438. | AC008727 | Genbank | 7582548 |
| 439. | AC008745 | Genbank | 9937749 |
| 440. | AC008753 | Genbank | 9937751 |
| 441. | AC008770 | Genbank | 9954580 |
| 442. | AC008794 | Genbank | 9558576 |
| 443. | AC008804 | Genbank | 9958011 |
| 444. | AC008934 | Genbank | 7158896 |
| 445. | AC008993 | Genbank | 7272070 |
| 446. | AC009060 | Genbank | 9690317 |
| 447. | AC009087 | Genbank | 7656694 |
| 448. | AC009134 | Genbank | 8886970 |
| 449. | AC009178 | Genbank | 6642684 |
| 450. | AC009227 | Genbank | 6289249 |
| 451. | AC009228 | Genbank | 10140829 |
| 452. | AC009229 | Genbank | 10716650 |
| 453. | AC009247 | Genbank | 6539155 |
| 454. | AC009305 | Genbank | 9857572 |
| 455. | AC009307 | Genbank | 9454630 |
| 456. | AC009332 | Genbank | 9789639 |
| 457. | AC009464 | Genbank | 6492471 |
| 458. | AC009470 | Genbank | 9454623 |
| 459. | AC009475 | Genbank | 8468974 |
| 460. | AC009478 | Genbank | 7770675 |
| 461. | AC009479 | Genbank | 10801458 |
| 462. | AC009487 | Genbank | 7321968 |
| 463. | AC009757 | Genbank | 8072443 |
| 464. | AC009946 | Genbank | 6604542 |
| 465. | AC009953 | Genbank | 8954248 |
| 466. | AC009958 | Genbank | 7408124 |
| 467. | AC010077 | Genbank | 5870275 |
| 468. | AC010102 | Genbank | 7670242 |
| 469. | AC010125 | Genbank | 7243876 |
| 470. | AC010135 | Genbank | 7243895 |
| 471. | AC010168 | Genbank | 6855156 |
| 472. | AC010200 | Genbank | 6492469 |
| 473. | AC010219 | Genbank | 7549619 |
| 474. | AC010285 | Genbank | 7651784 |
| 475. | AC010517 | Genbank | 6223625 |
| 476. | AC010525 | Genbank | 7248932 |
| 477. | AC010588 | Genbank | 8886974 |
| 478. | AC010645 | Genbank | 7670119 |
| 479. | AC010656 | Genbank | 9994134 |
| 480. | AC010792 | Genbank | 7363427 |
| 481. | AC011239 | Genbank | 9795955 |
| 482. | AC011248 | Genbank | 7658299 |
| 483. | AC011309 | Genbank | 7770057 |
| 484. | AC011310 | Genbank | 7243953 |
| 485. | AC011311 | Genbank | 6850247 |
| 486. | AC011331 | Genbank | 9929686 |
| 487. | AC011362 | Genbank | 6094545 |
| 488. | AC011450 | Genbank | 7670120 |
| 489. | AC011462 | Genbank | 7684381 |
| 490. | AC011465 | Genbank | 7211902 |
| 491. | AC011470 | Genbank | 9211204 |
| 492. | AC011599 | Genbank | 7113871 |
| 493. | AC011609 | Genbank | 7114193 |
| 494. | AC011815 | Genbank | 9280746 |
| 495. | AC012000 | Genbank | 8050952 |
| 496. | AC012061 | Genbank | 8747257 |
| 497. | AC012072 | Genbank | 7684569 |
| 498. | AC012083 | Genbank | 6649264 |
| 499. | AC012085 | Genbank | 6634755 |
| 500. | AC012156 | Genbank | 7363387 |
| 501. | AC012351 | Genbank | 9795658 |
| 502. | AC012472 | Genbank | 7715629 |
| 503. | AC012519 | Genbank | 9558566 |
| 504. | AC012614 | Genbank | 9625330 |
| 505. | AC013734 | Genbank | 9454645 |
| 506. | AC016138 | Genbank | 7021552 |
| 507. | AC016254 | Genbank | 9845100 |
| 508. | AC016395 | Genbank | 9929646 |
| 509. | AC016396 | Genbank | 7715613 |
| 510. | AC016656 | Genbank | 8099264 |
| 511. | AC017015 | Genbank | 10047965 |
| 512. | AC017092 | Genbank | 9887823 |
| 513. | AC017093 | Genbank | 9454641 |
| 514. | AC018514 | Genbank | 9802725 |
| 515. | AC018755 | Genbank | 9454515 |
| 516. | AC018758 | Genbank | 9954648 |
| 517. | AC018764 | Genbank | 11128366 |
| 518. | AC018797 | Genbank | 8747372 |
| 519. | AC018816 | Genbank | 9755439 |
| 520. | AC019058 | Genbank | 8748181 |
| 521. | AC019183 | Genbank | 7684557 |
| 522. | AC019215 | Genbank | 10048057 |
| 523. | AC019221 | Genbank | 10048073 |
| 524. | AC019226 | Genbank | 9858446 |
| 525. | AC020663 | Genbank | 6682593 |
| 526. | AC020728 | Genbank | 7770044 |
| 527. | AC020898 | Genbank | 10280746 |
| 528. | AC020910 | Genbank | 9558584 |
| 529. | AC020943 | Genbank | 7272102 |
| 530. | AC020946 | Genbank | 7670123 |
| 531. | AC022150 | Genbank | 8567765 |
| 532. | AC022336 | Genbank | 7684378 |
| 533. | AC022436 | Genbank | 9958018 |
| 534. | AC023473 | Genbank | 9581895 |
| 535. | AC024083 | Genbank | 9789634 |
| 536. | AC024085 | Genbank | 9295772 |
| 537. | AC024159 | Genbank | 9581896 |
| 538. | AC024561 | Genbank | 8099271 |
| 539. | AC024571 | Genbank | 10280747 |
| 540. | AC025613 | Genbank | 9964950 |
| 541. | AC026722 | Genbank | 10086451 |
| 542. | AC037199 | Genbank | 7527775 |
| 543. | AC040163 | Genbank | 8191116 |
| 544. | AC058791 | Genbank | 8810490 |
| 545. | AC061958 | Genbank | 8698765 |
| 546. | AC062033 | Genbank | 9858445 |
| 547. | AC069080 | Genbank | 9802814 |
| 548. | AC069298 | Genbank | 9964951 |
| 549. | AC074331 | Genbank | 9502399 |
| 550. | AC078899 | Genbank | 9789611 |
| 551. | AE000658 | Genbank | 2358019 |
| 552. | AF000364 | Genbank | 2697102 |
| 553. | AF000984 | Genbank | 2580553 |
| 554. | AF002697 | Genbank | 2511528 |
| 555. | AF002992 | Genbank | 2121300 |
| 556. | AF004162 | Genbank | 3046385 |
| 557. | AF006083 | Genbank | 2282031 |
| 558. | AF006501 | Genbank | 4836840 |
| 559. | AF010227 | Genbank | 2318005 |
| 560. | AF015043 | Genbank | 4102710 |
| 561. | AF016507 | Genbank | 2909776 |
| 562. | AF020202 | Genbank | 2431999 |
| 563. | AF028593 | Genbank | 2599081 |
| 564. | AF028832 | Genbank | 3287488 |
| 565. | AF030876 | Genbank | 3002589 |
| 566. | AF031403 | Genbank | 2920574 |
| 567. | AF032862 | Genbank | 3449363 |
| 568. | AF035298 | Genbank | 2661054 |
| 569. | AF035737 | Genbank | 2827179 |
| 570. | AF038172 | Genbank | 2795890 |
| 571. | AF038202 | Genbank | 2795923 |
| 572. | AF039656 | Genbank | 2773159 |
| 573. | AF041432 | Genbank | 2791803 |
| 574. | AF042090 | Genbank | 2829108 |
| 575. | AF042284 | Genbank | 5256829 |
| 576. | AF043045 | Genbank | 3282770 |
| 577. | AF052051 | Genbank | 5668857 |
| 578. | AF052113 | Genbank | 3360420 |
| 579. | AF052129 | Genbank | 3360438 |
| 580. | AF052135 | Genbank | 3360444 |
| 581. | AF054990 | Genbank | 3005703 |
| 582. | AF055032 | Genbank | 3005762 |
| 583. | AF059529 | Genbank | 4454663 |
| 584. | AF061736 | Genbank | 4335936 |
| 585. | AF061786 | Genbank | 3420027 |
| 586. | AF064245 | Genbank | 3930528 |
| 587. | AF064603 | Genbank | 3152659 |
| 588. | AF069506 | Genbank | 4959037 |
| 589. | AF070539 | Genbank | 3387896 |
| 590. | AF070556 | Genbank | 3387921 |
| 591. | AF070665 | Genbank | 4454705 |
| 592. | AF071771 | Genbank | 3243078 |
| 593. | AF072097 | Genbank | 5725511 |
| 594. | AF072864 | Genbank | 5441951 |
| 595. | AF073298 | Genbank | 3641537 |
| 596. | AF075061 | Genbank | 3377602 |
| 597. | AF077050 | Genbank | 4689147 |
| 598. | AF077202 | Genbank | 4679017 |
| 599. | AF077205 | Genbank | 4679023 |
| 600. | AF078848 | Genbank | 5531810 |
| 601. | AF078932 | Genbank | 5725528 |
| 602. | AF080092 | Genbank | 4322311 |
| 603. | AF083106 | Genbank | 7555470 |
| 604. | AF083441 | Genbank | 5813822 |
| 605. | AF084225 | Genbank | 4249652 |
| 606. | AF085832 | Genbank | 3483146 |
| 607. | AF086210 | Genbank | 3483555 |
| 608. | AF086334 | Genbank | 3483679 |
| 609. | AF086420 | Genbank | 3483765 |
| 610. | AF087481 | Genbank | 4322487 |
| 611. | AF088851 | Genbank | 9621743 |
| 612. | AF090936 | Genbank | 6690236 |
| 613. | AF091083 | Genbank | 3860003 |
| 614. | AF091882 | Genbank | 8515078 |
| 615. | AF097832 | Genbank | 5231268 |
| 616. | AF099137 | Genbank | 4566496 |
| 617. | AF099989 | Genbank | 3851170 |
| 618. | AF100755 | Genbank | 5410295 |
| 619. | AF104669 | Genbank | 4160446 |
| 620. | AF106656 | Genbank | 3983438 |
| 621. | AF107406 | Genbank | 5531905 |
| 622. | AF110774 | Genbank | 6523794 |
| 623. | AF111167 | Genbank | 4572570 |
| 624. | AF113016 | Genbank | 6642755 |
| 625. | AF113514 | Genbank | 6002685 |
| 626. | AF113680 | Genbank | 6855605 |
| 627. | AF113696 | Genbank | 6855628 |
| 628. | AF116606 | Genbank | 7959715 |
| 629. | AF116679 | Genbank | 7959856 |
| 630. | AF118090 | Genbank | 6650825 |
| 631. | AF118092 | Genbank | 6650829 |
| 632. | AF118808 | Genbank | 11131781 |
| 633. | AF119863 | Genbank | 7770162 |
| 634. | AF119897 | Genbank | 7770230 |
| 635. | AF119905 | Genbank | 7770246 |
| 636. | AF124523 | Genbank | 4325309 |
| 637. | AF124731 | Genbank | 4262558 |
| 638. | AF126962 | Genbank | 6469610 |
| 639. | AF131215 | Genbank | 8272457 |
| 640. | AF131216 | Genbank | 8272458 |
| 641. | AF131752 | Genbank | 4406570 |
| 642. | AF131777 | Genbank | 4406602 |
| 643. | AF131820 | Genbank | 4406655 |
| 644. | AF134583 | Genbank | 6066199 |
| 645. | AF135405 | Genbank | 7622401 |
| 646. | AF135830 | Genbank | 6179884 |
| 647. | AF141347 | Genbank | 4929133 |
| 648. | AF147271 | Genbank | 5823152 |
| 649. | AF147334 | Genbank | 4761685 |
| 650. | AF148457 | Genbank | 6164673 |
| 651. | AF151075 | Genbank | 7106871 |
| 652. | AF151103 | Genbank | 5758136 |
| 653. | AF151897 | Genbank | 4929746 |
| 654. | AF152363 | Genbank | 5669134 |
| 655. | AF152462 | Genbank | 5565976 |
| 656. | AF155114 | Genbank | 5360122 |
| 657. | AF155330 | Genbank | 5107389 |
| 658. | AF156098 | Genbank | 5070697 |
| 659. | AF161381 | Genbank | 6841175 |
| 660. | AF162130 | Genbank | 6997271 |
| 661. | AF165138 | Genbank | 5499744 |
| 662. | AF168787 | Genbank | 7239175 |
| 663. | AF168956 | Genbank | 5702387 |
| 664. | AF173003 | Genbank | 7329978 |
| 665. | AF178030 | Genbank | 6539738 |
| 666. | AF182002 | Genbank | 7648682 |
| 667. | AF187554 | Genbank | 6653225 |
| 668. | AF192784 | Genbank | 6572963 |
| 669. | AF195417 | Genbank | 6118554 |
| 670. | AF200465 | Genbank | 6690789 |
| 671. | AF201077 | Genbank | 6456748 |
| 672. | AF201950 | Genbank | 6563297 |
| 673. | AF202445 | Genbank | 9081980 |
| 674. | AF203815 | Genbank | 6979641 |
| 675. | AF207955 | Genbank | 6513835 |
| 676. | AF212220 | Genbank | 9437506 |
| 677. | AF217490 | Genbank | 8650408 |
| 678. | AF217506 | Genbank | 7688954 |
| 679. | AF220053 | Genbank | 7689012 |
| 680. | AF225898 | Genbank | 7021527 |
| 681. | AF227509 | Genbank | 6960196 |
| 682. | AF227948 | Genbank | 10803732 |
| 683. | AF239727 | Genbank | 7271932 |
| 684. | AF240627 | Genbank | 7263183 |
| 685. | AF254983 | Genbank | 7671239 |
| 686. | AF261085 | Genbank | 9802301 |
| 687. | AF265340 | Genbank | 8038131 |
| 688. | AF281906 | Genbank | 9828571 |
| 689. | AF282851 | Genbank | 9837244 |
| 690. | AF282904 | Genbank | 9885649 |
| 691. | AF292100 | Genbank | 9896485 |
| 692. | AF302505 | Genbank | 10242358 |
| 693. | AJ001612 | Genbank | 2407908 |
| 694. | AJ002385 | Genbank | 2597930 |
| 695. | AJ006973 | Genbank | 3319952 |
| 696. | AJ006996 | Genbank | 3378627 |
| 697. | AJ010069 | Genbank | 3483012 |
| 698. | AJ010395 | Genbank | 5542013 |
| 699. | AJ011930 | Genbank | 3859769 |
| 700. | AJ012166 | Genbank | 6562360 |
| 701. | AJ131959 | Genbank | 9650706 |
| 702. | AJ132695 | Genbank | 8574037 |
| 703. | AJ222700 | Genbank | 2665384 |
| 704. | AJ223352 | Genbank | 3255996 |
| 705. | AJ225782 | Genbank | 4165269 |
| 706. | AJ238374 | Genbank | 6634418 |
| 707. | AJ246003 | Genbank | 6273492 |
| 708. | AJ277892 | Genbank | 8249466 |
| 709. | AJ293009 | Genbank | 9967553 |
| 710. | AJ400717 | Genbank | 7573518 |
| 711. | AK000022 | Genbank | 7019835 |
| 712. | AK000086 | Genbank | 7019944 |
| 713. | AK000129 | Genbank | 7020013 |
| 714. | AK000379 | Genbank | 7020429 |
| 715. | AK000462 | Genbank | 7020566 |
| 716. | AK000464 | Genbank | 7020570 |
| 717. | AK000762 | Genbank | 7021052 |
| 718. | AK000799 | Genbank | 7021103 |
| 719. | AK000827 | Genbank | 7021141 |
| 720. | AK001088 | Genbank | 7022136 |
| 721. | AK001192 | Genbank | 7022292 |
| 722. | AK001313 | Genbank | 7022490 |
| 723. | AK001401 | Genbank | 7022634 |
| 724. | AK001569 | Genbank | 7022902 |
| 725. | AK001690 | Genbank | 7023105 |
| 726. | AK001777 | Genbank | 7023259 |
| 727. | AK001875 | Genbank | 7023414 |
| 728. | AK001878 | Genbank | 7023418 |
| 729. | AK001981 | Genbank | 7023587 |
| 730. | AK024888 | Genbank | 10437301 |
| 731. | AL008583 | Genbank | 4160195 |
| 732. | AL008628 | Genbank | 3094986 |
| 733. | AL008728 | Genbank | 3820992 |
| 734. | AL008730 | Genbank | 2842415 |
| 735. | AL009050 | Genbank | 6018756 |
| 736. | AL009182 | Genbank | 6624884 |
| 737. | AL009657 | Genbank | 2664820 |
| 738. | AL020994 | Genbank | 3355593 |
| 739. | AL020995 | Genbank | 10862821 |
| 740. | AL020996 | Genbank | 6599063 |
| 741. | AL021069 | Genbank | 2853183 |
| 742. | AL021329 | Genbank | 3873468 |
| 743. | AL021368 | Genbank | 3080468 |
| 744. | AL021396 | Genbank | 10190811 |
| 745. | AL021528 | Genbank | 3115987 |
| 746. | AL021578 | Genbank | 10242473 |
| 747. | AL021579 | Genbank | 4375984 |
| 748. | AL021707 | Genbank | 4582132 |
| 749. | AL021920 | Genbank | 3264535 |
| 750. | AL021939 | Genbank | 3135969 |
| 751. | AL022067 | Genbank | 3395491 |
| 752. | AL022144 | Genbank | 3421038 |
| 753. | AL022149 | Genbank | 4455439 |
| 754. | AL022153 | Genbank | 3319674 |
| 755. | AL022312 | Genbank | 4914501 |
| 756. | AL022320 | Genbank | 4914513 |
| 757. | AL022325 | Genbank | 3242175 |
| 758. | AL022393 | Genbank | 3046744 |
| 759. | AL022398 | Genbank | 3355547 |
| 760. | AL022721 | Genbank | 3367610 |
| 761. | AL022726 | Genbank | 3676217 |
| 762. | AL023284 | Genbank | 3355875 |
| 763. | AL023584 | Genbank | 3790154 |
| 764. | AL023753 | Genbank | 3702426 |
| 765. | AL024474 | Genbank | 3395511 |
| 766. | AL024498 | Genbank | 5924006 |
| 767. | AL024508 | Genbank | 3445456 |
| 768. | AL030999 | Genbank | 4490890 |
| 769. | AL031003 | Genbank | 4007185 |
| 770. | AL031120 | Genbank | 3805922 |
| 771. | AL031177 | Genbank | 4071056 |
| 772. | AL031224 | Genbank | 3947808 |
| 773. | AL031259 | Genbank | 3790132 |
| 774. | AL031281 | Genbank | 4826460 |
| 775. | AL031282 | Genbank | 3860395 |
| 776. | AL031287 | Genbank | 4914533 |
| 777. | AL031311 | Genbank | 3947674 |
| 778. | AL031315 | Genbank | 3820998 |
| 779. | AL031390 | Genbank | 5002609 |
| 780. | AL031447 | Genbank | 4826431 |
| 781. | AL031584 | Genbank | 3980333 |
| 782. | AL031591 | Genbank | 6006484 |
| 783. | AL031651 | Genbank | 6065866 |
| 784. | AL031655 | Genbank | 5360979 |
| 785. | AL031656 | Genbank | 5360983 |
| 786. | AL031660 | Genbank | 7160951 |
| 787. | AL031662 | Genbank | 9716901 |
| 788. | AL031666 | Genbank | 10198598 |
| 789. | AL031667 | Genbank | 10198629 |
| 790. | AL031668 | Genbank | 10198603 |
| 791. | AL031670 | Genbank | 4469083 |
| 792. | AL031682 | Genbank | 9795187 |
| 793. | AL031685 | Genbank | 9368423 |
| 794. | AL031716 | Genbank | 4826474 |
| 795. | AL031730 | Genbank | 4090216 |
| 796. | AL031775 | Genbank | 4071041 |
| 797. | AL031777 | Genbank | 10198609 |
| 798. | AL031780 | Genbank | 4140349 |
| 799. | AL031785 | Genbank | 3687446 |
| 800. | AL033397 | Genbank | 4902626 |
| 801. | AL033539 | Genbank | 6465824 |
| 802. | AL034347 | Genbank | 6911927 |
| 803. | AL034421 | Genbank | 10198621 |
| 804. | AL034551 | Genbank | 11125134 |
| 805. | AL034553 | Genbank | 5419653 |
| 806. | AL034554 | Genbank | 4585646 |
| 807. | AL034555 | Genbank | 4455444 |
| 808. | AL035071 | Genbank | 5002606 |
| 809. | AL035411 | Genbank | 6136940 |
| 810. | AL035415 | Genbank | 6522966 |
| 811. | AL035467 | Genbank | 6522967 |
| 812. | AL035468 | Genbank | 4581349 |
| 813. | AL035652 | Genbank | 10242468 |
| 814. | AL035653 | Genbank | 5764011 |
| 815. | AL035667 | Genbank | 5295847 |
| 816. | AL035683 | Genbank | 7288039 |
| 817. | AL035690 | Genbank | 5441633 |
| 818. | AL035692 | Genbank | 4775607 |
| 819. | AL035699 | Genbank | 4826515 |
| 820. | AL035705 | Genbank | 7159723 |
| 821. | AL049246 | Genbank | 4499983 |
| 822. | AL049471 | Genbank | 4500266 |
| 823. | AL049540 | Genbank | 5441688 |
| 824. | AL049542 | Genbank | 7406726 |
| 825. | AL049545 | Genbank | 5002650 |
| 826. | AL049564 | Genbank | 4902757 |
| 827. | AL049570 | Genbank | 4938316 |
| 828. | AL049589 | Genbank | 5679567 |
| 829. | AL049636 | Genbank | 5531522 |
| 830. | AL049691 | Genbank | 6018787 |
| 831. | AL049696 | Genbank | 5931893 |
| 832. | AL049715 | Genbank | 9581791 |
| 833. | AL049766 | Genbank | 5763746 |
| 834. | AL049767 | Genbank | 10198636 |
| 835. | AL049776 | Genbank | 6681704 |
| 836. | AL049820 | Genbank | 8247261 |
| 837. | AL049829 | Genbank | 8217859 |
| 838. | AL049835 | Genbank | 5708093 |
| 839. | AL049874 | Genbank | 7838310 |
| 840. | AL049951 | Genbank | 4884198 |
| 841. | AL050072 | Genbank | 4884304 |
| 842. | AL050137 | Genbank | 4884149 |
| 843. | AL050139 | Genbank | 4884349 |
| 844. | AL050325 | Genbank | 5830470 |
| 845. | AL050331 | Genbank | 5668655 |
| 846. | AL050343 | Genbank | 6137008 |
| 847. | AL050347 | Genbank | 4902836 |
| 848. | AL050349 | Genbank | 8655549 |
| 849. | AL078463 | Genbank | 5777575 |
| 850. | AL078581 | Genbank | 6967288 |
| 851. | AL078584 | Genbank | 5650668 |
| 852. | AL078587 | Genbank | 5918399 |
| 853. | AL078605 | Genbank | 7406515 |
| 854. | AL078614 | Genbank | 6425557 |
| 855. | AL078621 | Genbank | 6013067 |
| 856. | AL078623 | Genbank | 6522988 |
| 857. | AL078634 | Genbank | 6523636 |
| 858. | AL078645 | Genbank | 8218091 |
| 859. | AL079303 | Genbank | 7798987 |
| 860. | AL079342 | Genbank | 6018784 |
| 861. | AL080092 | Genbank | 5262512 |
| 862. | AL080234 | Genbank | 5262727 |
| 863. | AL080242 | Genbank | 5911841 |
| 864. | AL080250 | Genbank | 5804917 |
| 865. | AL080276 | Genbank | 5763753 |
| 866. | AL080277 | Genbank | 5738683 |
| 867. | AL096701 | Genbank | 5912599 |
| 868. | AL096773 | Genbank | 5918011 |
| 869. | AL096794 | Genbank | 5596739 |
| 870. | AL096800 | Genbank | 7242638 |
| 871. | AL096817 | Genbank | 5881344 |
| 872. | AL096821 | Genbank | 7009579 |
| 873. | AL096870 | Genbank | 8346725 |
| 874. | AL096888 | Genbank | 9581828 |
| 875. | AL109612 | Genbank | 5881342 |
| 876. | AL109627 | Genbank | 6742145 |
| 877. | AL109628 | Genbank | 8176898 |
| 878. | AL109755 | Genbank | 6165524 |
| 879. | AL109827 | Genbank | 6706902 |
| 880. | AL109828 | Genbank | 8218114 |
| 881. | AL109865 | Genbank | 6911646 |
| 882. | AL109914 | Genbank | 6594176 |
| 883. | AL109920 | Genbank | 7406500 |
| 884. | AL109921 | Genbank | 9588678 |
| 885. | AL109923 | Genbank | 8894632 |
| 886. | AL109930 | Genbank | 7838241 |
| 887. | AL109939 | Genbank | 6523730 |
| 888. | AL109976 | Genbank | 7406502 |
| 889. | AL110122 | Genbank | 5805143 |
| 890. | AL110167 | Genbank | 5817072 |
| 891. | AL110197 | Genbank | 5817115 |
| 892. | AL110297 | Genbank | 5817256 |
| 893. | AL110588 | Genbank | 5830289 |
| 894. | AL117187 | Genbank | 8217869 |
| 895. | AL117334 | Genbank | 7248316 |
| 896. | AL117342 | Genbank | 6580447 |
| 897. | AL117461 | Genbank | 5911922 |
| 898. | AL117519 | Genbank | 5912035 |
| 899. | AL117596 | Genbank | 5912164 |
| 900. | AL117621 | Genbank | 5912202 |
| 901. | AL118511 | Genbank | 9801559 |
| 902. | AL118519 | Genbank | 9187342 |
| 903. | AL118524 | Genbank | 7635676 |
| 904. | AL121579 | Genbank | 7271127 |
| 905. | AL121580 | Genbank | 6456854 |
| 906. | AL121585 | Genbank | 8248733 |
| 907. | AL121591 | Genbank | 9581797 |
| 908. | AL121612 | Genbank | 11191233 |
| 909. | AL121652 | Genbank | 7159615 |
| 910. | AL121675 | Genbank | 8388706 |
| 911. | AL121694 | Genbank | 6318179 |
| 912. | AL121790 | Genbank | 8919824 |
| 913. | AL121809 | Genbank | 8018146 |
| 914. | AL121825 | Genbank | 6911603 |
| 915. | AL121845 | Genbank | 8246778 |
| 916. | AL121846 | Genbank | 6630794 |
| 917. | AL121891 | Genbank | 9367201 |
| 918. | AL121896 | Genbank | 8218066 |
| 919. | AL121904 | Genbank | 7263976 |
| 920. | AL121909 | Genbank | 8218087 |
| 921. | AL121914 | Genbank | 9864672 |
| 922. | AL121928 | Genbank | 8247025 |
| 923. | AL121944 | Genbank | 7406632 |
| 924. | AL121958 | Genbank | 8247268 |
| 925. | AL121963 | Genbank | 7161783 |
| 926. | AL122072 | Genbank | 6102870 |
| 927. | AL122125 | Genbank | 8574298 |
| 928. | AL132640 | Genbank | 8574299 |
| 929. | AL132642 | Genbank | 8217878 |
| 930. | AL132656 | Genbank | 8439394 |
| 931. | AL132708 | Genbank | 7009592 |
| 932. | AL132715 | Genbank | 8217879 |
| 933. | AL132777 | Genbank | 6562026 |
| 934. | AL132822 | Genbank | 7838255 |
| 935. | AL132827 | Genbank | 8217885 |
| 936. | AL132986 | Genbank | 7159620 |
| 937. | AL132992 | Genbank | 6996086 |
| 938. | AL132994 | Genbank | 6491712 |
| 939. | AL133009 | Genbank | 6453414 |
| 940. | AL133074 | Genbank | 6453517 |
| 941. | AL133153 | Genbank | 9368785 |
| 942. | AL133173 | Genbank | 8217417 |
| 943. | AL133215 | Genbank | 7228177 |
| 944. | AL133228 | Genbank | 8217426 |
| 945. | AL133240 | Genbank | 8346743 |
| 946. | AL133241 | Genbank | 7630038 |
| 947. | AL133245 | Genbank | 7159621 |
| 948. | AL133260 | Genbank | 8439041 |
| 949. | AL133289 | Genbank | 6729371 |
| 950. | AL133330 | Genbank | 9650514 |
| 951. | AL133340 | Genbank | 7330646 |
| 952. | AL133353 | Genbank | 6706037 |
| 953. | AL133367 | Genbank | 7708222 |
| 954. | AL133370 | Genbank | 8248722 |
| 955. | AL133372 | Genbank | 7271128 |
| 956. | AL133380 | Genbank | 8217443 |
| 957. | AL133396 | Genbank | 6562003 |
| 958. | AL133415 | Genbank | 7160477 |
| 959. | AL133418 | Genbank | 8039190 |
| 960. | AL133439 | Genbank | 6562638 |
| 961. | AL133444 | Genbank | 7708223 |
| 962. | AL133445 | Genbank | 6691783 |
| 963. | AL133453 | Genbank | 7009594 |
| 964. | AL133454 | Genbank | 8919826 |
| 965. | AL133545 | Genbank | 9368987 |
| 966. | AL133551 | Genbank | 9407715 |
| 967. | AL135744 | Genbank | 6682291 |
| 968. | AL135787 | Genbank | 9588110 |
| 969. | AL135790 | Genbank | 9864450 |
| 970. | AL135838 | Genbank | 9187470 |
| 971. | AL135911 | Genbank | 9864452 |
| 972. | AL135999 | Genbank | 7799026 |
| 973. | AL136000 | Genbank | 7940101 |
| 974. | AL136097 | Genbank | 7799632 |
| 975. | AL136107 | Genbank | 9944117 |
| 976. | AL136179 | Genbank | 8649149 |
| 977. | AL136223 | Genbank | 9662907 |
| 978. | AL136228 | Genbank | 8894183 |
| 979. | AL136294 | Genbank | 8452637 |
| 980. | AL136295 | Genbank | 6850939 |
| 981. | AL136315 | Genbank | 8248843 |
| 982. | AL136365 | Genbank | 8246868 |
| 983. | AL136370 | Genbank | 8018015 |
| 984. | AL136381 | Genbank | 9801296 |
| 985. | AL136418 | Genbank | 7710966 |
| 986. | AL136438 | Genbank | 7406537 |
| 987. | AL136452 | Genbank | 7009497 |
| 988. | AL136504 | Genbank | 7018294 |
| 989. | AL136981 | Genbank | 9621476 |
| 990. | AL136985 | Genbank | 9542710 |
| 991. | AL136987 | Genbank | 9843896 |
| 992. | AL136999 | Genbank | 9581551 |
| 993. | AL137020 | Genbank | 10086020 |
| 994. | AL137100 | Genbank | 6983534 |
| 995. | AL137129 | Genbank | 7009596 |
| 996. | AL137141 | Genbank | 8217499 |
| 997. | AL137164 | Genbank | 7019736 |
| 998. | AL137226 | Genbank | 7708225 |
| 999. | AL137302 | Genbank | 6807766 |
| 1000. | AL137412 | Genbank | 6807964 |
| 1001. | AL137681 | Genbank | 6807931 |
| 1002. | AL137730 | Genbank | 6808260 |
| 1003. | AL137784 | Genbank | 9863487 |
| 1004. | AL137798 | Genbank | 9187169 |
| 1005. | AL137800 | Genbank | 9926422 |
| 1006. | AL137802 | Genbank | 8452475 |
| 1007. | AL137861 | Genbank | 9187172 |
| 1008. | AL138499 | Genbank | 8217926 |
| 1009. | AL138752 | Genbank | 8452480 |
| 1010. | AL138761 | Genbank | 8573811 |
| 1011. | AL138775 | Genbank | 8246884 |
| 1012. | AL138832 | Genbank | 9856663 |
| 1013. | AL138836 | Genbank | 10185444 |
| 1014. | AL138954 | Genbank | 8978058 |
| 1015. | AL138976 | Genbank | 7799028 |
| 1016. | AL138996 | Genbank | 7413824 |
| 1017. | AL139001 | Genbank | 9187180 |
| 1018. | AL139008 | Genbank | 8574139 |
| 1019. | AL139099 | Genbank | 8217934 |
| 1020. | AL139120 | Genbank | 7739105 |
| 1021. | AL139186 | Genbank | 9856677 |
| 1022. | AL139279 | Genbank | 9716311 |
| 1023. | AL139375 | Genbank | 9367942 |
| 1024. | AL139803 | Genbank | 8670911 |
| 1025. | AL157469 | Genbank | 7018485 |
| 1026. | AL157713 | Genbank | 10045328 |
| 1027. | AL157756 | Genbank | 7329704 |
| 1028. | AL157773 | Genbank | 9944137 |
| 1029. | AL157789 | Genbank | 8217949 |
| 1030. | AL157792 | Genbank | 7327842 |
| 1031. | AL157879 | Genbank | 8217606 |
| 1032. | AL157915 | Genbank | 7710968 |
| 1033. | AL158167 | Genbank | 10045340 |
| 1034. | AL158832 | Genbank | 9944141 |
| 1035. | AL160058 | Genbank | 9366919 |
| 1036. | AL160191 | Genbank | 7708226 |
| 1037. | AL160233 | Genbank | 7799784 |
| 1038. | AL160234 | Genbank | 8217960 |
| 1039. | AL160236 | Genbank | 7708227 |
| 1040. | AL160414 | Genbank | 9863596 |
| 1041. | AL161622 | Genbank | 10045356 |
| 1042. | AL161669 | Genbank | 8247492 |
| 1043. | AL161952 | Genbank | 7328002 |
| 1044. | AL161965 | Genbank | 7328021 |
| 1045. | AL162010 | Genbank | 7328036 |
| 1046. | AL162086 | Genbank | 7328174 |
| 1047. | AL162151 | Genbank | 8655500 |
| 1048. | AL162291 | Genbank | 9407802 |
| 1049. | AL162390 | Genbank | 8894273 |
| 1050. | AL162811 | Genbank | 8546786 |
| 1051. | AL163011 | Genbank | 8217990 |
| 1052. | AL163202 | Genbank | 7717242 |
| 1053. | AL163203 | Genbank | 7717244 |
| 1054. | AL163204 | Genbank | 7717247 |
| 1055. | AL163252 | Genbank | 7717317 |
| 1056. | AL163279 | Genbank | 7717362 |
| 1057. | AL163284 | Genbank | 7717380 |
| 1058. | AL163285 | Genbank | 7717384 |
| 1059. | AL163302 | Genbank | 7717445 |
| 1060. | AL163932 | Genbank | 8546788 |
| 1061. | AL352977 | Genbank | 8246738 |
| 1062. | AL353194 | Genbank | 9368012 |
| 1063. | AL353753 | Genbank | 9988303 |
| 1064. | AL353759 | Genbank | 8745068 |
| 1065. | AL353771 | Genbank | 8894296 |
| 1066. | AL353812 | Genbank | 8670913 |
| 1067. | AL354720 | Genbank | 8979470 |
| 1068. | AL354751 | Genbank | 9187237 |
| 1069. | AL354766 | Genbank | 10185557 |
| 1070. | AL354950 | Genbank | 9908994 |
| 1071. | AL355178 | Genbank | 9369121 |
| 1072. | AL355476 | Genbank | 10045405 |
| 1073. | AL357153 | Genbank | 8919854 |
| 1074. | AL357374 | Genbank | 9368149 |
| 1075. | AL358214 | Genbank | 9926659 |
| 1076. | AL359212 | Genbank | 8546790 |
| 1077. | AL359234 | Genbank | 8546796 |
| 1078. | AL359397 | Genbank | 8574316 |
| 1079. | AL389887 | Genbank | 9944194 |
| 1080. | AL390039 | Genbank | 10186780 |
| 1081. | AL391071 | Genbank | 9716842 |
| 1082. | AP000010 | Genbank | 4666256 |
| 1083. | AP000011 | Genbank | 4666257 |
| 1084. | AP000037 | Genbank | 3132347 |
| 1085. | AP000038 | Genbank | 3132348 |
| 1086. | AP000046 | Genbank | 3132356 |
| 1087. | AP000051 | Genbank | 3132361 |
| 1088. | AP000089 | Genbank | 4730830 |
| 1089. | AP000173 | Genbank | 4827138 |
| 1090. | AP000344 | Genbank | 5103007 |
| 1091. | AP000345 | Genbank | 5103008 |
| 1092. | AP000350 | Genbank | 5103013 |
| 1093. | AP000354 | Genbank | 5103017 |
| 1094. | AP000426 | Genbank | 8698836 |
| 1095. | AP000456 | Genbank | 6683121 |
| 1096. | AP000493 | Genbank | 5926660 |
| 1097. | AP000495 | Genbank | 5926677 |
| 1098. | AP000496 | Genbank | 5926683 |
| 1099. | AP000525 | Genbank | 5931503 |
| 1100. | AP000528 | Genbank | 5931506 |
| 1101. | AP000555 | Genbank | 5931541 |
| 1102. | AP000697 | Genbank | 6712194 |
| 1103. | AP000957 | Genbank | 7077200 |
| 1104. | AP000958 | Genbank | 6705920 |
| 1105. | AP001053 | Genbank | 6693603 |
| 1106. | AP001137 | Genbank | 6970361 |
| 1107. | AP001207 | Genbank | 8698839 |
| 1108. | AP001208 | Genbank | 8698840 |
| 1109. | AP001343 | Genbank | 7209831 |
| 1110. | AP001468 | Genbank | 7768596 |
| 1111. | AP001469 | Genbank | 7768597 |
| 1112. | AP001477 | Genbank | 7768605 |
| 1113. | AP001599 | Genbank | 7670553 |
| 1114. | AP001610 | Genbank | 7670564 |
| 1115. | AP001759 | Genbank | 7768685 |
| 1116. | AP002022 | Genbank | 7798582 |
| 1117. | AP002028 | Genbank | 9293863 |
| 1118. | AX012184 | Genbank | 9998287 |
| 1119. | AX013102 | Genbank | 10040268 |
| 1120. | AX013726 | Genbank | 10040436 |
| 1121. | AX013777 | Genbank | 10040487 |
| 1122. | AX013784 | Genbank | 10040494 |
| 1123. | AX014092 | Genbank | 10040562 |
| 1124. | AX014301 | Genbank | 10040655 |
| 1125. | AX014326 | Genbank | 10040680 |
| 1126. | AX014829 | Genbank | 10041096 |
| 1127. | AX014868 | Genbank | 10041135 |
| 1128. | AX015381 | Genbank | 10041361 |
| 1129. | AX015384 | Genbank | 10041364 |
| 1130. | AX015912 | Genbank | 10041655 |
| 1131. | AX024599 | Genbank | 10184739 |
| 1132. | AY007086 | Genbank | 9955975 |
| 1133. | AY007110 | Genbank | 9956004 |
| 1134. | AY007115 | Genbank | 9956010 |
| 1135. | D00761 | Genbank | 220025 |
| 1136. | D12765 | Genbank | 219610 |
| 1137. | D14664 | Genbank | 285952 |
| 1138. | D14696 | Genbank | 285962 |
| 1139. | D17171 | Genbank | 598659 |
| 1140. | D17391 | Genbank | 440365 |
| 1141. | D26445 | Genbank | 452520 |
| 1142. | D28126 | Genbank | 559316 |
| 1143. | D29805 | Genbank | 474986 |
| 1144. | D31890 | Genbank | 505107 |
| 1145. | D38048 | Genbank | 1531532 |
| 1146. | D38073 | Genbank | 862331 |
| 1147. | D38305 | Genbank | 1580723 |
| 1148. | D45248 | Genbank | 1008914 |
| 1149. | D50663 | Genbank | 1747307 |
| 1150. | D67031 | Genbank | 2696053 |
| 1151. | D79983 | Genbank | 1136383 |
| 1152. | D79986 | Genbank | 1136389 |
| 1153. | D80004 | Genbank | 1136423 |
| 1154. | D84145 | Genbank | 2114143 |
| 1155. | D86993 | Genbank | 2114214 |
| 1156. | D87018 | Genbank | 2114280 |
| 1157. | D87116 | Genbank | 1711248 |
| 1158. | D87666 | Genbank | 1620016 |
| 1159. | D87953 | Genbank | 1596166 |
| 1160. | D88357 | Genbank | 3126638 |
| 1161. | E02628 | Genbank | 2170856 |
| 1162. | E05934 | Genbank | 2174121 |
| 1163. | E12495 | Genbank | 3251328 |
| 1164. | G05042 | Genbank | 722000 |
| 1165. | G12671 | Genbank | 1113284 |
| 1166. | G28850 | Genbank | 1526743 |
| 1167. | G51878 | Genbank | 5223205 |
| 1168. | G52432 | Genbank | 5223609 |
| 1169. | G52625 | Genbank | 5223952 |
| 1170. | G53306 | Genbank | 5224483 |
| 1171. | G55786 | Genbank | 6120955 |
| 1172. | J00128 | Genbank | 182425 |
| 1173. | J00129 | Genbank | 182429 |
| 1174. | J00200 | Genbank | 188411 |
| 1175. | J02683 | Genbank | 179246 |
| 1176. | J02843 | Genbank | 181355 |
| 1177. | J03746 | Genbank | 183655 |
| 1178. | J04611 | Genbank | 178649 |
| 1179. | K00558 | Genbank | 340020 |
| 1180. | K01763 | Genbank | 184316 |
| 1181. | K01886 | Genbank | 182820 |
| 1182. | K02215 | Genbank | 178639 |
| 1183. | K02569 | Genbank | 182441 |
| 1184. | L06237 | Genbank | 437000 |
| 1185. | L10400 | Genbank | 187509 |
| 1186. | L12535 | Genbank | 434050 |
| 1187. | L12711 | Genbank | 388890 |
| 1188. | L15702 | Genbank | 291921 |
| 1189. | L17411 | Genbank | 1199855 |
| 1190. | L22569 | Genbank | 348706 |
| 1191. | L31610 | Genbank | 1220360 |
| 1192. | L47647 | Genbank | 1000861 |
| 1193. | L76465 | Genbank | 1203983 |
| 1194. | L77701 | Genbank | 1280205 |
| 1195. | M10036 | Genbank | 339840 |
| 1196. | M11146 | Genbank | 182504 |
| 1197. | M11167 | Genbank | 337381 |
| 1198. | M11353 | Genbank | 184092 |
| 1199. | M11725 | Genbank | 181067 |
| 1200. | M12387 | Genbank | 184325 |
| 1201. | M13692 | Genbank | 178256 |
| 1202. | M14333 | Genbank | 181171 |
| 1203. | M17885 | Genbank | 190231 |
| 1204. | M19997 | Genbank | 181968 |
| 1205. | M21154 | Genbank | 178517 |
| 1206. | M24194 | Genbank | 187701 |
| 1207. | M27504 | Genbank | 339809 |
| 1208. | M37583 | Genbank | 184059 |
| 1209. | M61841 | Genbank | 187042 |
| 1210. | M62839 | Genbank | 178856 |
| 1211. | M64241 | Genbank | 190813 |
| 1212. | M64779 | Genbank | 181449 |
| 1213. | M67480 | Genbank | 190364 |
| 1214. | M74491 | Genbank | 178161 |
| 1215. | M75099 | Genbank | 337369 |
| 1216. | M83665 | Genbank | 184235 |
| 1217. | M88006 | Genbank | 178522 |
| 1218. | M88108 | Genbank | 189499 |
| 1219. | M90309 | Genbank | 182643 |
| 1220. | M96995 | Genbank | 181975 |
| 1221. | S54005 | Genbank | 264772 |
| 1222. | S69272 | Genbank | 546087 |
| 1223. | S74506 | Genbank | 807059 |
| 1224. | U03494 | Genbank | 476098 |
| 1225. | U07550 | Genbank | 469170 |
| 1226. | U12465 | Genbank | 562073 |
| 1227. | U13369 | Genbank | 555853 |
| 1228. | U14972 | Genbank | 550024 |
| 1229. | U22431 | Genbank | 881345 |
| 1230. | U26424 | Genbank | 1203795 |
| 1231. | U29589 | Genbank | 903978 |
| 1232. | U38784 | Genbank | 1574947 |
| 1233. | U38864 | Genbank | 1055340 |
| 1234. | U43747 | Genbank | 1237438 |
| 1235. | U50078 | Genbank | 4220427 |
| 1236. | U51903 | Genbank | 1262925 |
| 1237. | U58196 | Genbank | 1388159 |
| 1238. | U67122 | Genbank | 1762972 |
| 1239. | U73168 | Genbank | 1613899 |
| 1240. | U73634 | Genbank | 1737189 |
| 1241. | U73641 | Genbank | 2281065 |
| 1242. | U73645 | Genbank | 1737200 |
| 1243. | U79271 | Genbank | 1710237 |
| 1244. | U79282 | Genbank | 1710254 |
| 1245. | U80017 | Genbank | 1737211 |
| 1246. | U82613 | Genbank | 1773066 |
| 1247. | U82828 | Genbank | 2304970 |
| 1248. | U89935 | Genbank | 2190717 |
| 1249. | U90339 | Genbank | 1906010 |
| 1250. | U90920 | Genbank | 2522321 |
| 1251. | U91323 | Genbank | 3582311 |
| 1252. | X01037 | Genbank | 36086 |
| 1253. | X03205 | Genbank | 36162 |
| 1254. | X04098 | Genbank | 28338 |
| 1255. | X07315 | Genbank | 35578 |
| 1256. | X07369 | Genbank | 35319 |
| 1257. | X15187 | Genbank | 37260 |
| 1258. | X15327 | Genbank | 28699 |
| 1259. | X15341 | Genbank | 1197215 |
| 1260. | X51445 | Genbank | 36320 |
| 1261. | X52851 | Genbank | 30167 |
| 1262. | X52882 | Genbank | 311380 |
| 1263. | X55733 | Genbank | 288099 |
| 1264. | X56932 | Genbank | 23690 |
| 1265. | X58141 | Genbank | 28381 |
| 1266. | X60221 | Genbank | 509290 |
| 1267. | X62534 | Genbank | 32332 |
| 1268. | X62741 | Genbank | 36058 |
| 1269. | X63564 | Genbank | 36123 |
| 1270. | X70649 | Genbank | 3123573 |
| 1271. | X71973 | Genbank | 311699 |
| 1272. | X75535 | Genbank | 1644300 |
| 1273. | X83218 | Genbank | 1008079 |
| 1274. | X84908 | Genbank | 1502344 |
| 1275. | X85117 | Genbank | 1161563 |
| 1276. | X97064 | Genbank | 1296663 |
| 1277. | X99585 | Genbank | 1770518 |
| 1278. | Y00815 | Genbank | 34266 |
| 1279. | Y08614 | Genbank | 1707479 |
| 1280. | Y10196 | Genbank | 1834504 |
| 1281. | Y15286 | Genbank | 2584788 |
| 1282. | Z11692 | Genbank | 31107 |
| 1283. | Z12006 | Genbank | 28884 |
| 1284. | Z12011 | Genbank | 28888 |
| 1285. | Z14070 | Genbank | 35822 |
| 1286. | Z15005 | Genbank | 29864 |
| 1287. | Z35093 | Genbank | 895848 |
| 1288. | Z47087 | Genbank | 860989 |
| 1289. | Z48501 | Genbank | 693936 |
| 1290. | Z48570 | Genbank | 695580 |
| 1291. | Z64407 | Genbank | 1037229 |
| 1292. | Z69374 | Genbank | 1183912 |
| 1293. | Z73678 | Genbank | 1770487 |
| 1294. | Z75889 | Genbank | 1430780 |
| 1295. | Z81007 | Genbank | 1621233 |
| 1296. | Z81310 | Genbank | 1638831 |
| 1297. | Z81370 | Genbank | 1657265 |
| 1298. | Z82193 | Genbank | 2370074 |
| 1299. | Z82203 | Genbank | 3164070 |
| 1300. | Z82215 | Genbank | 3135984 |
| 1301. | Z82899 | Genbank | 1945756 |
| 1302. | Z83826 | Genbank | 4902653 |
| 1303. | Z83848 | Genbank | 3164072 |
| 1304. | Z84469 | Genbank | 3204451 |
| 1305. | Z84478 | Genbank | 3171880 |
| 1306. | Z84480 | Genbank | 3150089 |
| 1307. | Z84488 | Genbank | 2058316 |
| 1308. | Z84496 | Genbank | 2094801 |
| 1309. | Z84813 | Genbank | 1841925 |
| 1310. | Z86061 | Genbank | 2253035 |
| 1311. | Z86062 | Genbank | 2058315 |
| 1312. | Z86064 | Genbank | 3191972 |
| 1313. | Z92542 | Genbank | 4490841 |
| 1314. | Z92543 | Genbank | 3164068 |
| 1315. | Z94044 | Genbank | 2245342 |
| 1316. | Z94056 | Genbank | 2326510 |
| 1317. | Z94721 | Genbank | 2462374 |
| 1318. | Z95325 | Genbank | 5596940 |
| 1319. | Z95329 | Genbank | 2225930 |
| 1320. | Z97055 | Genbank | 2916859 |
| 1321. | Z97353 | Genbank | 4455632 |
| 1322. | Z97989 | Genbank | 2760549 |
| 1323. | Z98048 | Genbank | 2582746 |
| 1324. | Z98051 | Genbank | 5679749 |
| 1325. | Z98052 | Genbank | 3947833 |
| 1326. | Z99129 | Genbank | 3281967 |
| 1327. | Z99496 | Genbank | 2780183 |
| 1328. | Z99716 | Genbank | 4456457 |
| 1329. | Z99758 | Genbank | 4775628 |
| 1330. | Z99943 | Genbank | 2887308 |
| 1331. | AA001348 | EST | 1437452 |
| 1332. | AA001609 | EST | 1445186 |
| 1333. | AA001792 | EST | 1445606 |
| 1334. | AA009518 | EST | 1470717 |
| 1335. | AA010257 | EST | 1471434 |
| 1336. | AA010721 | EST | 1471768 |
| 1337. | AA022854 | EST | 1486934 |
| 1338. | AA025727 | EST | 1491403 |
| 1339. | AA026455 | EST | 1492355 |
| 1340. | AA026758 | EST | 1492556 |
| 1341. | AA028108 | EST | 1494195 |
| 1342. | AA029269 | EST | 1496710 |
| 1343. | AA033875 | EST | 1505693 |
| 1344. | AA037177 | EST | 1512321 |
| 1345. | AA040621 | EST | 1517034 |
| 1346. | AA042812 | EST | 1522467 |
| 1347. | AA046429 | EST | 1526340 |
| 1348. | AA059132 | EST | 1552002 |
| 1349. | AA059337 | EST | 1553194 |
| 1350. | AA064860 | EST | 1558981 |
| 1351. | AA079880 | EST | 1618772 |
| 1352. | AA081040 | EST | 1622975 |
| 1353. | AA090004 | EST | 1636552 |
| 1354. | AA098807 | EST | 1644778 |
| 1355. | AA098864 | EST | 1645048 |
| 1356. | AA101090 | EST | 1647618 |
| 1357. | AA101533 | EST | 1648619 |
| 1358. | AA112516 | EST | 1665065 |
| 1359. | AA121394 | EST | 1679017 |
| 1360. | AA122400 | EST | 1678776 |
| 1361. | AA127600 | EST | 1686916 |
| 1362. | AA128006 | EST | 1687286 |
| 1363. | AA130532 | EST | 1691971 |
| 1364. | AA132709 | EST | 1694217 |
| 1365. | AA134011 | EST | 1691079 |
| 1366. | AA134380 | EST | 1691874 |
| 1367. | AA136725 | EST | 1697935 |
| 1368. | AA147747 | EST | 1717310 |
| 1369. | AA148511 | EST | 1721555 |
| 1370. | AA151993 | EST | 1720967 |
| 1371. | AA152181 | EST | 1721233 |
| 1372. | AA152273 | EST | 1721676 |
| 1373. | AA159500 | EST | 1735043 |
| 1374. | AA160288 | EST | 1734865 |
| 1375. | AA164613 | EST | 1740791 |
| 1376. | AA165312 | EST | 1740540 |
| 1377. | AA165705 | EST | 1741756 |
| 1378. | AA166853 | EST | 1745071 |
| 1379. | AA186876 | EST | 1775011 |
| 1380. | AA190363 | EST | 1779057 |
| 1381 | AA194656 | EST | 1784413 |
| 1382. | AA195259 | EST | 1784959 |
| 1383. | AA205647 | EST | 1803639 |
| 1384. | AA229399 | EST | 1851232 |
| 1385. | AA232557 | EST | 1855429 |
| 1386. | AA233204 | EST | 1856216 |
| 1387. | AA255879 | EST | 1891420 |
| 1388. | AA278702 | EST | 1920022 |
| 1389. | AA290683 | EST | 1938946 |
| 1390. | AA292008 | EST | 1939985 |
| 1391. | AA296394 | EST | 1948728 |
| 1392. | AA301248 | EST | 1953642 |
| 1393. | AA306545 | EST | 1958874 |
| 1394. | AA329981 | EST | 1982265 |
| 1395. | AA343038 | EST | 1995274 |
| 1396. | AA349726 | EST | 2002046 |
| 1397. | AA393934 | EST | 2046903 |
| 1398. | AA400644 | EST | 2054515 |
| 1399. | AA400670 | EST | 2054604 |
| 1400. | AA404289 | EST | 2059013 |
| 1401. | AA404343 | EST | 2059068 |
| 1402. | AA417168 | EST | 2077249 |
| 1403. | AA417603 | EST | 2079431 |
| 1404. | AA418632 | EST | 2080442 |
| 1405. | AA421015 | EST | 2099848 |
| 1406. | AA425334 | EST | 2107359 |
| 1407. | AA425576 | EST | 2106332 |
| 1408. | AA428977 | EST | 2110527 |
| 1409. | AA429263 | EST | 2110787 |
| 1410. | AA442415 | EST | 2154293 |
| 1411. | AA447163 | EST | 2159828 |
| 1412. | AA447254 | EST | 2159919 |
| 1413. | AA447745 | EST | 2161415 |
| 1414. | AA453183 | EST | 2166852 |
| 1415. | AA454977 | EST | 2177753 |
| 1416. | AA459016 | EST | 2183923 |
| 1417. | AA459850 | EST | 2184757 |
| 1418. | AA460963 | EST | 2186083 |
| 1419. | AA461070 | EST | 2186190 |
| 1420. | AA464646 | EST | 2189530 |
| 1421. | AA485253 | EST | 2214472 |
| 1422. | AA490342 | EST | 2219515 |
| 1423. | AA491222 | EST | 2220395 |
| 1424. | AA492457 | EST | 2222019 |
| 1425. | AA495988 | EST | 2229309 |
| 1426. | AA523678 | EST | 2264606 |
| 1427. | AA525477 | EST | 2264499 |
| 1428. | AA534419 | EST | 2278672 |
| 1429. | AA534948 | EST | 2279201 |
| 1430. | AA536208 | EST | 2280461 |
| 1431. | AA541677 | EST | 2288111 |
| 1432. | AA551141 | EST | 2321393 |
| 1433. | AA553908 | EST | 2324447 |
| 1434. | AA554358 | EST | 2324897 |
| 1435. | AA555312 | EST | 2325851 |
| 1436. | AA557205 | EST | 2327682 |
| 1437. | AA564512 | EST | 2336151 |
| 1438. | AA574235 | EST | 2348750 |
| 1439. | AA580413 | EST | 2355740 |
| 1440. | AA584705 | EST | 2369314 |
| 1441. | AA595070 | EST | 2410420 |
| 1442. | AA595339 | EST | 2410689 |
| 1443. | AA602550 | EST | 2436484 |
| 1444. | AA639587 | EST | 2563366 |
| 1445. | AA648922 | EST | 2575351 |
| 1446. | AA654316 | EST | 2590470 |
| 1447. | AA657518 | EST | 2593672 |
| 1448. | AA659388 | EST | 2595542 |
| 1449. | AA669052 | EST | 2630551 |
| 1450. | AA669657 | EST | 2631156 |
| 1451. | AA687631 | EST | 2674537 |
| 1452. | AA701639 | EST | 2704804 |
| 1453. | AA702186 | EST | 2705299 |
| 1454. | AA704332 | EST | 2714250 |
| 1455. | AA725239 | EST | 2742946 |
| 1456. | AA730280 | EST | 2753492 |
| 1457. | AA732780 | EST | 2754139 |
| 1458. | AA740821 | EST | 2779413 |
| 1459. | AA749446 | EST | 2789404 |
| 1460. | AA775180 | EST | 2834514 |
| 1461. | AA779948 | EST | 2839279 |
| 1462. | AA833879 | EST | 2907478 |
| 1463. | AA834017 | EST | 2907616 |
| 1464. | AA843539 | EST | 2930057 |
| 1465. | AA854398 | EST | 2941936 |
| 1466. | AA854815 | EST | 2942353 |
| 1467. | AA856697 | EST | 2944999 |
| 1468. | AA878415 | EST | 2987380 |
| 1469. | AA885898 | EST | 3001006 |
| 1470. | AA910353 | EST | 3049643 |
| 1471. | AA913153 | EST | 3052545 |
| 1472. | AA916867 | EST | 3056259 |
| 1473. | AA923175 | EST | 3070484 |
| 1474. | AA926713 | EST | 3075610 |
| 1475. | AA928802 | EST | 3078159 |
| 1476. | AA939115 | EST | 3099028 |
| 1477. | AA947871 | EST | 3109124 |
| 1478. | AA971639 | EST | 3146929 |
| 1479. | AA974055 | EST | 3149235 |
| 1480. | AA992985 | EST | 3179530 |
| 1481. | AA994848 | EST | 3181337 |
| 1482. | AF001542 | EST | 2529714 |
| 1483. | AF035745 | EST | 2662514 |
| 1484. | AF150245 | EST | 5133681 |
| 1485. | AI000675 | EST | 3191229 |
| 1486. | AI015610 | EST | 3229946 |
| 1487. | AI016237 | EST | 3230573 |
| 1488. | AI026708 | EST | 3246196 |
| 1489. | AI027428 | EST | 3244944 |
| 1490. | AI032033 | EST | 3250245 |
| 1491. | AI034044 | EST | 3254997 |
| 1492. | AI052376 | EST | 3308367 |
| 1493. | AI052390 | EST | 3308381 |
| 1494. | AI052439 | EST | 3308430 |
| 1495. | AI052745 | EST | 3308736 |
| 1496. | AI056320 | EST | 3330186 |
| 1497. | AI064691 | EST | 6358963 |
| 1498. | AI064863 | EST | 6359135 |
| 1499. | AI064942 | EST | 6359214 |
| 1500. | AI095282 | EST | 3434258 |
| 1501. | AI095447 | EST | 3434423 |
| 1502. | AI096490 | EST | 3445984 |
| 1503. | AI096820 | EST | 3446402 |
| 1504. | AI114630 | EST | 6359975 |
| 1505. | AI114651 | EST | 6359996 |
| 1506. | AI126126 | EST | 3594640 |
| 1507. | AI127227 | EST | 3595741 |
| 1508. | AI133386 | EST | 6360702 |
| 1509. | AI139947 | EST | 3647404 |
| 1510. | AI146568 | EST | 3674250 |
| 1511. | AI174394 | EST | 3721247 |
| 1512. | AI185118 | EST | 3735756 |
| 1513. | AI207411 | EST | 6361419 |
| 1514. | AI224992 | EST | 3807705 |
| 1515. | AI247193 | EST | 3842590 |
| 1516. | AI251694 | EST | 3848223 |
| 1517. | AI268571 | EST | 3887738 |
| 1518. | AI269205 | EST | 3888372 |
| 1519. | AI271786 | EST | 3890953 |
| 1520. | AI272718 | EST | 3894986 |
| 1521. | AI274508 | EST | 3896776 |
| 1522. | AI274769 | EST | 3897043 |
| 1523. | AI278317 | EST | 3900585 |
| 1524. | AI280747 | EST | 3918980 |
| 1525. | AI282039 | EST | 3920272 |
| 1526. | AI287890 | EST | 3927643 |
| 1527. | AI290667 | EST | 3933441 |
| 1528. | AI298904 | EST | 3958558 |
| 1529. | AI302808 | EST | 3962154 |
| 1530. | AI310332 | EST | 4005203 |
| 1531. | AI354283 | EST | 4094436 |
| 1532. | AI358226 | EST | 4109847 |
| 1533. | AI360459 | EST | 4112080 |
| 1534. | AI375191 | EST | 4175181 |
| 1535. | AI375615 | EST | 4175605 |
| 1536. | AI400580 | EST | 4243667 |
| 1537. | AI420634 | EST | 4266565 |
| 1538. | AI423356 | EST | 4269287 |
| 1539. | AI432656 | EST | 4283443 |
| 1540. | AI436456 | EST | 4281845 |
| 1541. | AI445131 | EST | 4286848 |
| 1542. | AI457521 | EST | 4310390 |
| 1543. | AI469532 | EST | 4331622 |
| 1544. | AI475134 | EST | 4328179 |
| 1545. | AI523884 | EST | 4438019 |
| 1546. | AI538017 | EST | 4452152 |
| 1547. | AI547277 | EST | 4464765 |
| 1548. | AI557226 | EST | 4489589 |
| 1549. | AI559531 | EST | 4509736 |
| 1550. | AI559947 | EST | 4510152 |
| 1551. | AI564247 | EST | 4522704 |
| 1552. | AI564835 | EST | 4523292 |
| 1553. | AI619779 | EST | 4628905 |
| 1554. | AI620639 | EST | 4629765 |
| 1555. | AI623190 | EST | 4648115 |
| 1556. | AI624454 | EST | 4649385 |
| 1557. | AI625316 | EST | 4650247 |
| 1558. | AI634701 | EST | 4686031 |
| 1559. | AI635164 | EST | 4686494 |
| 1560. | AI672534 | EST | 4852265 |
| 1561. | AI682391 | EST | 4892573 |
| 1562. | AI682779 | EST | 4892961 |
| 1563. | AI684591 | EST | 4895885 |
| 1564. | AI689840 | EST | 4900934 |
| 1565. | AI692476 | EST | 4969816 |
| 1566. | AI693153 | EST | 4970493 |
| 1567. | AI693263 | EST | 4970603 |
| 1568. | AI700092 | EST | 4987992 |
| 1569. | AI700720 | EST | 4988620 |
| 1570. | AI701845 | EST | 4989745 |
| 1571. | AI738816 | EST | 5100797 |
| 1572. | AI743652 | EST | 5111940 |
| 1573. | AI754461 | EST | 5132725 |
| 1574. | AI761117 | EST | 5176784 |
| 1575. | AI762801 | EST | 5178468 |
| 1576. | AI768545 | EST | 5235054 |
| 1577. | AI796795 | EST | 5362258 |
| 1578. | AI797716 | EST | 5363188 |
| 1579. | AI804292 | EST | 5369764 |
| 1580. | AI805556 | EST | 5392122 |
| 1581. | AI810813 | EST | 5397471 |
| 1582. | AI816806 | EST | 5435885 |
| 1583. | AI829333 | EST | 5450004 |
| 1584. | AI857560 | EST | 5511176 |
| 1585. | AI859799 | EST | 5513404 |
| 1586. | AI860630 | EST | 5514246 |
| 1587. | AI880736 | EST | 5514352 |
| 1588. | AI863067 | EST | 5527174 |
| 1589. | AI866602 | EST | 5530709 |
| 1590. | AI868969 | EST | 5542937 |
| 1591. | AI871317 | EST | 5545287 |
| 1592. | AI872545 | EST | 5546594 |
| 1593. | AI879221 | EST | 5553270 |
| 1594. | AI880389 | EST | 5554438 |
| 1595. | AI884783 | EST | 5589947 |
| 1596. | AI885591 | EST | 5590755 |
| 1597. | AI886415 | EST | 5591579 |
| 1598. | AI887948 | EST | 5593112 |
| 1599. | AI904067 | EST | 6494454 |
| 1600. | AI904309 | EST | 6494696 |
| 1601. | AI907465 | EST | 6497895 |
| 1602. | AI927993 | EST | 5663957 |
| 1603. | AI929217 | EST | 5665181 |
| 1604. | AI948772 | EST | 5741082 |
| 1605. | AI951277 | EST | 5743587 |
| 1606. | AI962969 | EST | 5755682 |
| 1607. | AI969269 | EST | 5766087 |
| 1608. | AI972614 | EST | 5769440 |
| 1609. | AL042900 | EST | 5422335 |
| 1610. | AL120189 | EST | 5926088 |
| 1611. | AL120455 | EST | 5926354 |
| 1612. | AV645545 | EST | 9866559 |
| 1613. | AV647552 | EST | 9868566 |
| 1614. | AV651256 | EST | 9872270 |
| 1615. | AV656303 | EST | 9877317 |
| 1616. | AV684275 | EST | 10286138 |
| 1617. | AW006470 | EST | 5855248 |
| 1618. | AW013827 | EST | 5862584 |
| 1619. | AW027984 | EST | 5886740 |
| 1620. | AW051710 | EST | 5913992 |
| 1621. | AW063595 | EST | 8887532 |
| 1622. | AW073536 | EST | 6028534 |
| 1623. | AW083912 | EST | 6039064 |
| 1624. | AW083968 | EST | 6039120 |
| 1625. | AW129432 | EST | 6117376 |
| 1626. | AW131227 | EST | 6132834 |
| 1627. | AW151775 | EST | 6199673 |
| 1628. | AW173161 | EST | 6439109 |
| 1629. | AW237295 | EST | 6569684 |
| 1630. | AW263421 | EST | 6640237 |
| 1631. | AW268670 | EST | 6655700 |
| 1632. | AW274640 | EST | 6661670 |
| 1633. | AW302677 | EST | 6712357 |
| 1634. | AW361153 | EST | 6865803 |
| 1635. | AW377225 | EST | 6881888 |
| 1636. | AW392056 | EST | 6896715 |
| 1637. | AW402883 | EST | 6921653 |
| 1638. | AW440855 | EST | 6976086 |
| 1639. | AW451105 | EST | 6991881 |
| 1640. | AW511111 | EST | 7149189 |
| 1641. | AW512504 | EST | 7150582 |
| 1642. | AW514850 | EST | 7152932 |
| 1643. | AW753352 | EST | 7668284 |
| 1644. | AW806898 | EST | 7899892 |
| 1645. | AW807747 | EST | 7900741 |
| 1646. | AW809305 | EST | 7902299 |
| 1647. | AW864069 | EST | 7998119 |
| 1648. | AW873105 | EST | 8007158 |
| 1649. | AW884334 | EST | 8046346 |
| 1650. | AW889976 | EST | 8054181 |
| 1651. | AW936566 | EST | 8111972 |
| 1652. | AW945629 | EST | 8123384 |
| 1653. | AW951818 | EST | 8141497 |
| 1654. | AW953381 | EST | 8143064 |
| 1655. | AW955131 | EST | 8144814 |
| 1656. | AW958100 | EST | 8147783 |
| 1857. | AW958344 | EST | 8148027 |
| 1658. | AW959608 | EST | 8149292 |
| 1659. | AW961068 | EST | 8150647 |
| 1660. | AW971643 | EST | 8161489 |
| 1661. | AW973231 | EST | 8163077 |
| 1662. | AW975279 | EST | 8166488 |
| 1663. | AW975603 | EST | 8166819 |
| 1664. | AW991955 | EST | 8252030 |
| 1665. | BE000581 | EST | 8260814 |
| 1666. | BE046509 | EST | 8363562 |
| 1667. | BE047758 | EST | 8364811 |
| 1668. | BE047766 | EST | 8364819 |
| 1669. | BE062077 | EST | 8406727 |
| 1670. | BE082802 | EST | 8473107 |
| 1671. | BE086006 | EST | 8476399 |
| 1672. | BE143562 | EST | 8606283 |
| 1673. | BE163198 | EST | 8625919 |
| 1674. | BE176845 | EST | 8639574 |
| 1675. | BE185081 | EST | 8664265 |
| 1676. | BE222728 | EST | 8910046 |
| 1677. | BE244882 | EST | 9096712 |
| 1678. | BE276767 | EST | 9151730 |
| 1679. | BE277504 | EST | 9152474 |
| 1680. | BE277605 | EST | 9152576 |
| 1681. | BE300078 | EST | 9183826 |
| 1682. | BE315321 | EST | 9145964 |
| 1683. | BE315408 | EST | 9146166 |
| 1684. | BE327329 | EST | 9201105 |
| 1685. | BE336870 | EST | 9189255 |
| 1686. | BE390367 | EST | 9335732 |
| 1687. | BE394923 | EST | 9340288 |
| 1688. | BE538492 | EST | 9767137 |
| 1689. | BE539033 | EST | 9767678 |
| 1690. | BE550909 | EST | 9792601 |
| 1691. | BE614974 | EST | 9896573 |
| 1692. | BE646434 | EST | 9970745 |
| 1693. | BE670259 | EST | 10030800 |
| 1694. | BE670566 | EST | 10031107 |
| 1695. | BE676699 | EST | 10037240 |
| 1696. | BE736674 | EST | 10150666 |
| 1697. | BE737615 | EST | 10151607 |
| 1698. | BE738997 | EST | 10152989 |
| 1699. | BE739157 | EST | 10153149 |
| 1700. | BE739423 | EST | 10153415 |
| 1701. | BE739764 | EST | 10153756 |
| 1702. | BE782705 | EST | 10203903 |
| 1703. | BE813522 | EST | 10245860 |
| 1704. | BE873774 | EST | 10322550 |
| 1705. | D20141 | EST | 501238 |
| 1706. | F23291 | EST | 3925692 |
| 1707. | H53413 | EST | 993560 |
| 1708. | H77782 | EST | 1055871 |
| 1709. | N25039 | EST | 1139189 |
| 1710. | N27865 | EST | 1142346 |
| 1711. | N73032 | EST | 1230136 |
| 1712. | R11660 | EST | 764395 |
| 1713. | R20950 | EST | 775731 |
| 1714. | R81264 | EST | 857867 |
| 1715. | R94664 | EST | 970059 |
| 1716. | T75153 | EST | 691915 |
| 1717. | T85745 | EST | 714097 |
| 1718. | T87878 | EST | 716230 |
| 1719. | U69197 | EST | 2739420 |
| 1720. | W32113 | EST | 1313154 |
| 1721. | W37520 | EST | 1319268 |
| 1722. | W47315 | EST | 1331954 |
| 1723. | W56081 | EST | 1357971 |
| 1724. | W56871 | EST | 1358767 |
| 1725. | W95023 | EST | 1424143 |
| 1726. | A26357 | NUCPATENT | 904918 |
| 1727. | T39279 | NUCPATENT | 647033 |
| 1728. | V82787 | NUCPATENT | N/A |
| 1729. | X07429 | NUCPATENT | 29524 |
| 1730. | Z15485 | NUCPATENT | N/A |
| 1731. | Z16636 | NUCPATENT | 23193 |
| 1732. | Z56704 | NUCPATENT | 1027935 |
| 1733. | Z86967 | NUCPATENT | 1883879 |
| 1734. | A02759 | Genbank | 345130 |
| 1735. | A14133 | Genbank | 490127 |
| 1736. | A16753 | Genbank | 512413 |
| 1737. | A69527 | Genbank | 4774176 |
| 1738. | AB000888 | Genbank | 2467297 |
| 1739. | AB002323 | Genbank | 2224590 |
| 1740. | AB002366 | Genbank | 2224676 |
| 1741. | AB002368 | Genbank | 2224680 |
| 1742. | AB002386 | Genbank | 2224716 |
| 1743. | AB002387 | Genbank | 2224718 |
| 1744. | AB003151 | Genbank | 3702678 |
| 1745. | AB006077 | Genbank | 2564010 |
| 1746. | AB007860 | Genbank | 2662080 |
| 1747. | AB007876 | Genbank | 2887452 |
| 1748. | AB011135 | Genbank | 3043649 |
| 1749. | AB011141 | Genbank | 3043661 |
| 1750. | AB011178 | Genbank | 3043735 |
| 1751. | AB011399 | Genbank | 3452571 |
| 1752. | AB011792 | Genbank | 3786311 |
| 1753. | AB014085 | Genbank | 5672597 |
| 1754. | AB014519 | Genbank | 3327051 |
| 1755. | AB014527 | Genbank | 3327067 |
| 1756. | AB014596 | Genbank | 3327205 |
| 1757. | AB014888 | Genbank | 3402484 |
| 1758. | AB015331 | Genbank | 3970851 |
| 1759. | AB015639 | Genbank | 5821139 |
| 1760. | AB015752 | Genbank | 3746106 |
| 1761. | AB016043 | Genbank | 4586865 |
| 1762. | AB017018 | Genbank | 4512253 |
| 1763. | AB018272 | Genbank | 3882178 |
| 1764. | AB018281 | Genbank | 3882196 |
| 1765. | AB018282 | Genbank | 3882198 |
| 1766. | AB018310 | Genbank | 3882254 |
| 1767. | AB018357 | Genbank | 6009486 |
| 1768. | AB019568 | Genbank | 3885371 |
| 1769. | AB020335 | Genbank | 6518494 |
| 1770. | AB020638 | Genbank | 4240150 |
| 1771. | AB020662 | Genbank | 4240198 |
| 1772. | AB020663 | Genbank | 4240200 |
| 1773. | AB020708 | Genbank | 4240290 |
| 1774. | AB020864 | Genbank | 4003384 |
| 1775. | AB020865 | Genbank | 4003385 |
| 1776. | AB020866 | Genbank | 4003386 |
| 1777. | AB020871 | Genbank | 4003391 |
| 1778. | AB020872 | Genbank | 4003392 |
| 1779. | AB021288 | Genbank | 4038732 |
| 1780. | AB022430 | Genbank | 4587270 |
| 1781. | AB023048 | Genbank | 5672603 |
| 1782. | AB023145 | Genbank | 4589487 |
| 1783. | AB023181 | Genbank | 4589571 |
| 1784. | AB023208 | Genbank | 4589625 |
| 1785. | AB024334 | Genbank | 6016837 |
| 1786. | AB027466 | Genbank | 6172220 |
| 1787. | AB028624 | Genbank | 5103045 |
| 1788. | AB028893 | Genbank | 6552364 |
| 1789. | AB028948 | Genbank | 5689386 |
| 1790. | AB028951 | Genbank | 5689392 |
| 1791. | AB028969 | Genbank | 5689428 |
| 1792. | AB028972 | Genbank | 5689434 |
| 1793. | AB033079 | Genbank | 6382025 |
| 1794. | AB033094 | Genbank | 6331212 |
| 1795. | AB037675 | Genbank | 9650961 |
| 1796. | AB037803 | Genbank | 7243144 |
| 1797. | AB040903 | Genbank | 7959200 |
| 1798. | AB040922 | Genbank | 7959238 |
| 1799. | AB040938 | Genbank | 7959270 |
| 1800. | AB041267 | Genbank | 7594731 |
| 1801. | AB041511 | Genbank | 9711368 |
| 1802. | AB041512 | Genbank | 9711431 |
| 1803. | AB042030 | Genbank | 9711448 |
| 1804. | AB044702 | Genbank | 8671558 |
| 1805. | AB045360 | Genbank | 8918544 |
| 1806. | AB045362 | Genbank | 8918546 |
| 1807. | AB046774 | Genbank | 10047172 |
| 1808. | AB046778 | Genbank | 10047180 |
| 1809. | AB046844 | Genbank | 10047324 |
| 1810. | AC000064 | Genbank | 1669369 |
| 1811. | AC000066 | Genbank | 3645943 |
| 1812. | AC000079 | Genbank | 3845380 |
| 1813. | AC000116 | Genbank | 1809229 |
| 1814. | AC000122 | Genbank | 2772559 |
| 1815. | AC000124 | Genbank | 1814192 |
| 1816. | AC000353 | Genbank | 6970735 |
| 1817. | AC000367 | Genbank | 2347066 |
| 1818. | AC000394 | Genbank | 2133911 |
| 1819. | AC000403 | Genbank | 2133864 |
| 1820. | AC001082 | Genbank | 1930895 |
| 1821. | AC001546 | Genbank | 1944727 |
| 1822. | AC002038 | Genbank | 2226439 |
| 1823. | AC002040 | Genbank | 2347081 |
| 1824. | AC002041 | Genbank | 2576343 |
| 1825. | AC002044 | Genbank | 2347079 |
| 1826. | AC002045 | Genbank | 2951945 |
| 1827. | AC002060 | Genbank | 7143420 |
| 1828. | AC002064 | Genbank | 2076723 |
| 1829. | AC002080 | Genbank | 2078455 |
| 1830. | AC002081 | Genbank | 2078453 |
| 1831. | AC002112 | Genbank | 2347181 |
| 1832. | AC002124 | Genbank | 2121320 |
| 1833. | AC002297 | Genbank | 2182280 |
| 1834. | AC002352 | Genbank | 3858888 |
| 1835. | AC002366 | Genbank | 2739349 |
| 1836. | AC002377 | Genbank | 2275192 |
| 1837. | AC002379 | Genbank | 2275190 |
| 1838. | AC002381 | Genbank | 2275186 |
| 1839. | AC002394 | Genbank | 2815550 |
| 1840. | AC002395 | Genbank | 3540146 |
| 1841. | AC002426 | Genbank | 2734102 |
| 1842. | AC002429 | Genbank | 2335067 |
| 1843. | AC002450 | Genbank | 2337884 |
| 1844. | AC002464 | Genbank | 2337864 |
| 1845. | AC002465 | Genbank | 2337862 |
| 1846. | AC002476 | Genbank | 2340101 |
| 1847. | AC002480 | Genbank | 2340098 |
| 1848. | AC002483 | Genbank | 3598729 |
| 1849. | AC002488 | Genbank | 2580480 |
| 1850. | AC002519 | Genbank | 2815551 |
| 1851. | AC002528 | Genbank | 2388554 |
| 1852. | AC002531 | Genbank | 2580476 |
| 1853. | AC002540 | Genbank | 2393736 |
| 1854. | AC002545 | Genbank | 2828781 |
| 1855. | AC002553 | Genbank | 3126783 |
| 1856. | AC003003 | Genbank | 2865207 |
| 1857. | AC003007 | Genbank | 2911728 |
| 1858. | AC003034 | Genbank | 3219338 |
| 1859. | AC003037 | Genbank | 2920805 |
| 1860. | AC003078 | Genbank | 2588631 |
| 1861. | AC003080 | Genbank | 2588627 |
| 1862. | AC003087 | Genbank | 2588617 |
| 1863. | AC003091 | Genbank | 2588612 |
| 1864. | AC003092 | Genbank | 2588611 |
| 1865. | AC003103 | Genbank | 2842782 |
| 1866. | AC003108 | Genbank | 2833632 |
| 1867. | AC003667 | Genbank | 2995482 |
| 1868. | AC003682 | Genbank | 3264845 |
| 1869. | AC003684 | Genbank | 3165399 |
| 1870. | AC003953 | Genbank | 2708755 |
| 1871. | AC003954 | Genbank | 2708754 |
| 1872. | AC003977 | Genbank | 3219341 |
| 1873. | AC003985 | Genbank | 2769692 |
| 1874. | AC003989 | Genbank | 2772538 |
| 1875. | AC003992 | Genbank | 2772533 |
| 1876. | AC003998 | Genbank | 2772568 |
| 1877. | AC003999 | Genbank | 2772566 |
| 1878. | AC004002 | Genbank | 2772560 |
| 1879. | AC004003 | Genbank | 2772557 |
| 1880. | AC004013 | Genbank | 2781380 |
| 1881. | AC004020 | Genbank | 3219330 |
| 1882. | AC004028 | Genbank | 2804348 |
| 1883. | AC004050 | Genbank | 4001528 |
| 1884. | AC004053 | Genbank | 3687218 |
| 1885. | AC004057 | Genbank | 3004542 |
| 1886. | AC004059 | Genbank | 4263637 |
| 1887. | AC004063 | Genbank | 3299825 |
| 1888. | AC004068 | Genbank | 3046280 |
| 1889. | AC004072 | Genbank | 2944110 |
| 1890. | AC004083 | Genbank | 2822159 |
| 1891. | AC004107 | Genbank | 2828770 |
| 1892. | AC004108 | Genbank | 3108047 |
| 1893. | AC004109 | Genbank | 2828788 |
| 1894. | AC004141 | Genbank | 2880080 |
| 1895. | AC004148 | Genbank | 3482960 |
| 1896. | AC004164 | Genbank | 2905827 |
| 1897. | AC004166 | Genbank | 8887011 |
| 1898. | AC004222 | Genbank | 3108042 |
| 1899. | AC004223 | Genbank | 3253129 |
| 1900. | AC004230 | Genbank | 4028938 |
| 1901. | AC004236 | Genbank | 2914668 |
| 1902. | AC004381 | Genbank | 2982169 |
| 1903. | AC004386 | Genbank | 3046272 |
| 1904. | AC004388 | Genbank | 3046271 |
| 1905. | AC004451 | Genbank | 2979604 |
| 1906. | AC004456 | Genbank | 2979597 |
| 1907. | AC004457 | Genbank | 2979596 |
| 1908. | AC004464 | Genbank | 3093420 |
| 1909. | AC004472 | Genbank | 2984582 |
| 1910. | AC004477 | Genbank | 3688107 |
| 1911. | AC004485 | Genbank | 2992497 |
| 1912. | AC004492 | Genbank | 2995606 |
| 1913. | AC004502 | Genbank | 2996637 |
| 1914. | AC004513 | Genbank | 3242735 |
| 1915. | AC004526 | Genbank | 3779035 |
| 1916. | AC004527 | Genbank | 4760422 |
| 1917. | AC004539 | Genbank | 3041851 |
| 1918. | AC004552 | Genbank | 3273378 |
| 1919. | AC004582 | Genbank | 3337307 |
| 1920. | AC004584 | Genbank | 3417305 |
| 1921. | AC004594 | Genbank | 3063516 |
| 1922. | AC004606 | Genbank | 3097870 |
| 1923. | AC004613 | Genbank | 3080664 |
| 1924. | AC004614 | Genbank | 3080662 |
| 1925. | AC004662 | Genbank | 3451369 |
| 1926. | AC004668 | Genbank | 3115345 |
| 1927. | AC004677 | Genbank | 3126881 |
| 1928. | AC004679 | Genbank | 3128155 |
| 1929. | AC004686 | Genbank | 3688105 |
| 1930. | AC004691 | Genbank | 3135285 |
| 1931. | AC004773 | Genbank | 3169212 |
| 1932. | AC004782 | Genbank | 3172145 |
| 1933. | AC004801 | Genbank | 4204244 |
| 1934. | AC004808 | Genbank | 3192566 |
| 1935. | AC004814 | Genbank | 5708498 |
| 1936. | AC004831 | Genbank | 3980559 |
| 1937. | AC004837 | Genbank | 3845417 |
| 1938. | AC004841 | Genbank | 4454523 |
| 1939. | AC004842 | Genbank | 4753286 |
| 1940. | AC004843 | Genbank | 3845416 |
| 1941. | AC004849 | Genbank | 3980553 |
| 1942. | AC004850 | Genbank | 4309890 |
| 1943. | AC004853 | Genbank | 3766130 |
| 1944. | AC004858 | Genbank | 6624125 |
| 1945. | AC004861 | Genbank | 3818412 |
| 1946. | AC004866 | Genbank | 3980548 |
| 1947. | AC004875 | Genbank | 3980546 |
| 1948. | AC004885 | Genbank | 4926909 |
| 1949. | AC004886 | Genbank | 4156188 |
| 1950. | AC004896 | Genbank | 3845414 |
| 1951. | AC004902 | Genbank | 5757546 |
| 1952. | AC004903 | Genbank | 4454521 |
| 1953. | AC004908 | Genbank | 4156179 |
| 1954. | AC004911 | Genbank | 3478665 |
| 1955. | AC004914 | Genbank | 4156177 |
| 1956. | AC004917 | Genbank | 4508144 |
| 1957. | AC004923 | Genbank | 4263742 |
| 1958. | AC004924 | Genbank | 4263741 |
| 1959. | AC004925 | Genbank | 4156174 |
| 1960. | AC004926 | Genbank | 4263559 |
| 1961. | AC004931 | Genbank | 3406053 |
| 1962. | AC004944 | Genbank | 4156165 |
| 1963. | AC004955 | Genbank | 4753267 |
| 1964. | AC004961 | Genbank | 5001540 |
| 1965. | AC004967 | Genbank | 5001539 |
| 1966. | AC004980 | Genbank | 9755474 |
| 1967. | AC004982 | Genbank | 3419846 |
| 1968. | AC004983 | Genbank | 4309885 |
| 1969. | AC004984 | Genbank | 3355524 |
| 1970. | AC004986 | Genbank | 4309812 |
| 1971. | AC004987 | Genbank | 5306306 |
| 1972. | AC004989 | Genbank | 3900851 |
| 1973. | AC004998 | Genbank | 5091651 |
| 1974. | AC004999 | Genbank | 3970963 |
| 1975. | AC005000 | Genbank | 9857564 |
| 1976. | AC005023 | Genbank | 3900847 |
| 1977. | AC005031 | Genbank | 3688109 |
| 1978. | AC005040 | Genbank | 4508119 |
| 1979. | AC005045 | Genbank | 4508117 |
| 1980. | AC005052 | Genbank | 10122134 |
| 1981. | AC005053 | Genbank | 3924666 |
| 1982. | AC005058 | Genbank | 4156135 |
| 1983. | AC005061 | Genbank | 4508115 |
| 1984. | AC005070 | Genbank | 3406049 |
| 1985. | AC005083 | Genbank | 4150930 |
| 1986. | AC005084 | Genbank | 3659503 |
| 1987. | AC005088 | Genbank | 4753221 |
| 1988. | AC005099 | Genbank | 4150927 |
| 1989. | AC005104 | Genbank | 4218027 |
| 1990. | AC005154 | Genbank | 3242763 |
| 1991. | AC005158 | Genbank | 5091650 |
| 1992. | AC005184 | Genbank | 3687200 |
| 1993. | AC005189 | Genbank | 3264580 |
| 1994. | AC005213 | Genbank | 3282167 |
| 1995. | AC005215 | Genbank | 3282165 |
| 1996. | AC005230 | Genbank | 4156158 |
| 1997. | AC005235 | Genbank | 5091645 |
| 1998. | AC005244 | Genbank | 3402739 |
| 1999. | AC005250 | Genbank | 3287719 |
| 2000. | AC005261 | Genbank | 3289984 |
| 2001. | AC005271 | Genbank | 3293212 |
| 2002. | AC005281 | Genbank | 4263751 |
| 2003. | AC005283 | Genbank | 7243875 |
| 2004. | AC005288 | Genbank | 3492893 |
| 2005. | AC005301 | Genbank | 7107554 |
| 2006. | AC005317 | Genbank | 3808082 |
| 2007. | AC005326 | Genbank | 3341714 |
| 2008. | AC005328 | Genbank | 3342735 |
| 2009. | AC005332 | Genbank | 3659494 |
| 2010. | AC005352 | Genbank | 3366560 |
| 2011. | AC005365 | Genbank | 3367509 |
| 2012. | AC005373 | GenbanK | 3367501 |
| 2013. | AC005392 | Genbank | 3399669 |
| 2014. | AC005401 | Genbank | 3402734 |
| 2015. | AC005409 | Genbank | 4249432 |
| 2016. | AC005476 | Genbank | 5732183 |
| 2017. | AC005497 | Genbank | 8570495 |
| 2018. | AC005518 | Genbank | 4263560 |
| 2019. | AC005520 | Genbank | 5001541 |
| 2020. | AC005521 | Genbank | 4156161 |
| 2021. | AC005532 | Genbank | 4156192 |
| 2022. | AC005548 | Genbank | 3687279 |
| 2023. | AC005611 | Genbank | 3540154 |
| 2024. | AC005629 | Genbank | 7243877 |
| 2025. | AC005630 | Genbank | 4159882 |
| 2026. | AC005701 | Genbank | 3757543 |
| 2027. | AC005703 | Genbank | 7740042 |
| 2028. | AC005723 | Genbank | 3660463 |
| 2029. | AC005730 | Genbank | 3779042 |
| 2030. | AC005738 | Genbank | 3687213 |
| 2031. | AC005771 | Genbank | 3757545 |
| 2032. | AC005778 | Genbank | 3702301 |
| 2033. | AC005783 | Genbank | 3702294 |
| 2034. | AC005817 | Genbank | 7923527 |
| 2035. | AC005829 | Genbank | 3873300 |
| 2036. | AC005856 | Genbank | 3849821 |
| 2037. | AC005874 | Genbank | 6249674 |
| 2038. | AC005876 | Genbank | 6249672 |
| 2039. | AC005879 | Genbank | 6249669 |
| 2040. | AC005880 | Genbank | 6249667 |
| 2041. | AC005899 | Genbank | 3935221 |
| 2042. | AC005905 | Genbank | 4090182 |
| 2043. | AC005909 | Genbank | 4165006 |
| 2044. | AC005968 | Genbank | 3907448 |
| 2045. | AC006001 | Genbank | 5708496 |
| 2046. | AC006002 | Genbank | 3983570 |
| 2047. | AC006016 | Genbank | 4508142 |
| 2048. | AC006017 | Genbank | 4508141 |
| 2049. | AC006033 | Genbank | 4309948 |
| 2050. | AC006035 | Genbank | 4309810 |
| 2051. | AC006036 | Genbank | 4753244 |
| 2052. | AC006040 | Genbank | 10801470 |
| 2053. | AC006042 | Genbank | 4508120 |
| 2054. | AC006045 | Genbank | 4753227 |
| 2055. | AC006050 | Genbank | 4079627 |
| 2056. | AC006052 | Genbank | 4827293 |
| 2057. | AC006055 | Genbank | 4071011 |
| 2058. | AC006059 | Genbank | 4544348 |
| 2059. | AC006084 | Genbank | 3953485 |
| 2060. | AC006145 | Genbank | 4454525 |
| 2061. | AC006151 | Genbank | 6094678 |
| 2062. | AC006157 | Genbank | 4314424 |
| 2063. | AC006195 | Genbank | 3983557 |
| 2064. | AC006226 | Genbank | 4454497 |
| 2065. | AC006239 | Genbank | 4544480 |
| 2066. | AC006249 | Genbank | 4062902 |
| 2067. | AC006253 | Genbank | 4309922 |
| 2068. | AC006257 | Genbank | 4092478 |
| 2069. | AC006287 | Genbank | 4210508 |
| 2070. | AC006296 | Genbank | 4827292 |
| 2071. | AC006299 | Genbank | 4106997 |
| 2072. | AC006317 | Genbank | 4827327 |
| 2073. | AC006320 | Genbank | 8747085 |
| 2074. | AC006323 | Genbank | 4753259 |
| 2075. | AC006332 | Genbank | 6358848 |
| 2076. | AC006333 | Genbank | 5523811 |
| 2077. | AC006337 | Genbank | 9581973 |
| 2078. | AC006344 | Genbank | 4508150 |
| 2079. | AC006347 | Genbank | 4309919 |
| 2080. | AC006350 | Genbank | 4508140 |
| 2081. | AC006352 | Genbank | 4753268 |
| 2082. | AC006353 | Genbank | 4699957 |
| 2083. | AC006355 | Genbank | 4753266 |
| 2084. | AC006359 | Genbank | 4753263 |
| 2085. | AC006368 | Genbank | 4753243 |
| 2086. | AC006369 | Genbank | 5757529 |
| 2087. | AC006374 | Genbank | 5454238 |
| 2088. | AC006377 | Genbank | 4753233 |
| 2089. | AC006387 | Genbank | 4926888 |
| 2090. | AC006396 | Genbank | 4156191 |
| 2091. | AC006397 | Genbank | 4156148 |
| 2092. | AC006430 | Genbank | 6453929 |
| 2093. | AC006443 | Genbank | 4204325 |
| 2094. | AC006458 | Genbank | 4508134 |
| 2095. | AC006460 | Genbank | 7243904 |
| 2096. | AC006463 | Genbank | 4753236 |
| 2097. | AC006465 | Genbank | 4508124 |
| 2098. | AC006477 | Genbank | 7243871 |
| 2099. | AC006480 | Genbank | 6289252 |
| 2100. | AC006484 | Genbank | 4263399 |
| 2101. | AC006487 | Genbank | 11128438 |
| 2102. | AC006501 | Genbank | 4309874 |
| 2103. | AC006504 | Genbank | 4220442 |
| 2104. | AC006515 | Genbank | 4558536 |
| 2105. | AC006530 | Genbank | 4680764 |
| 2106. | AC006572 | Genbank | 4309773 |
| 2107. | AC006597 | Genbank | 4512725 |
| 2108. | AC006602 | Genbank | 4263802 |
| 2109. | AC006840 | Genbank | 6227027 |
| 2110. | AC006924 | Genbank | 4314414 |
| 2111. | AC006925 | Genbank | 4417322 |
| 2112. | AC006947 | Genbank | 4406829 |
| 2113. | AC006948 | Genbank | 4689496 |
| 2114. | AC006960 | Genbank | 4337211 |
| 2115. | AC006987 | Genbank | 5123989 |
| 2116. | AC007009 | Genbank | 7322010 |
| 2117. | AC007023 | Genbank | 6289261 |
| 2118. | AC007035 | Genbank | 9887792 |
| 2119. | AC007040 | Genbank | 5306302 |
| 2120. | AC007041 | Genbank | 5708471 |
| 2121. | AC007051 | Genbank | 4432877 |
| 2122. | AC007055 | Genbank | 4885691 |
| 2123. | AC007056 | Genbank | 4966341 |
| 2124. | AC007057 | Genbank | 4680440 |
| 2125. | AC007068 | Genbank | 4926862 |
| 2126. | AC007097 | Genbank | 7770678 |
| 2127. | AC007115 | Genbank | 4895146 |
| 2128. | AC007131 | Genbank | 8748848 |
| 2129. | AC007161 | Genbank | 4508162 |
| 2130. | AC007199 | Genbank | 4558768 |
| 2131. | AC007216 | Genbank | 6806842 |
| 2132. | AC007225 | Genbank | 6715703 |
| 2133. | AC007250 | Genbank | 5836191 |
| 2134. | AC007253 | Genbank | 5649375 |
| 2135. | AC007271 | Genbank | 7408074 |
| 2136. | AC007285 | Genbank | 5931439 |
| 2137. | AC007286 | Genbank | 5819124 |
| 2138. | AC007319 | Genbank | 5001496 |
| 2139. | AC007360 | Genbank | 10440739 |
| 2140. | AC007384 | Genbank | 5708473 |
| 2141. | AC007404 | Genbank | 8469030 |
| 2142. | AC007446 | Genbank | 5069495 |
| 2143. | AC007463 | Genbank | 5708477 |
| 2144. | AC007541 | Genbank | 4982536 |
| 2145. | AC007543 | Genbank | 4926860 |
| 2146. | AC007544 | Genbank | 6094534 |
| 2147. | AC007551 | Genbank | 4809347 |
| 2148. | AC007560 | Genbank | 5306301 |
| 2149. | AC007563 | Genbank | 6289217 |
| 2150. | AC007567 | Genbank | 11120966 |
| 2151. | AC007568 | Genbank | 4835813 |
| 2152. | AC007639 | Genbank | 5091642 |
| 2153. | AC007671 | Genbank | 6600816 |
| 2154. | AC007679 | Genbank | 9795944 |
| 2155. | AC007680 | Genbank | 6598918 |
| 2156. | AC007682 | Genbank | 10440752 |
| 2157. | AC007684 | Genbank | 5836173 |
| 2158. | AC007686 | Genbank | 6466519 |
| 2159. | AC007707 | Genbank | 5764724 |
| 2160. | AC007738 | Genbank | 6094647 |
| 2161. | AC007878 | Genbank | 5732161 |
| 2162. | AC007880 | Genbank | 5931460 |
| 2163. | AC007883 | Genbank | 7622521 |
| 2164. | AC007966 | Genbank | 5836190 |
| 2165. | AC007969 | Genbank | 7534288 |
| 2166. | AC007993 | Genbank | 6693756 |
| 2167. | AC008013 | Genbank | 5801645 |
| 2168. | AC008014 | Genbank | 5815493 |
| 2169. | AC008039 | Genbank | 5454236 |
| 2170. | AC008056 | Genbank | 6456144 |
| 2171. | AC008066 | Genban | 10048003 |
| 2172. | AC008068 | Genbank | 8748885 |
| 2173. | AC008070 | Genbank | 8748906 |
| 2174. | AC008123 | Genbank | 6006046 |
| 2175. | AC008154 | Genbank | 6729061 |
| 2176. | AC008168 | Genbank | 6289251 |
| 2177. | AC008171 | Genbank | 10716651 |
| 2178. | AC008179 | Genbank | 5931440 |
| 2179. | AC008243 | Genbank | 8347984 |
| 2180. | AC008277 | Genbank | 10716648 |
| 2181. | AC008278 | Genbank | 7408138 |
| 2182. | AC008279 | Genbank | 7408141 |
| 2183. | AC008394 | Genbank | 6850300 |
| 2184. | AC008403 | Genbank | 9690314 |
| 2185. | AC008469 | Genbank | 7019608 |
| 2186. | AC008471 | Genbank | 8134855 |
| 2187. | AC008483 | Genbank | 9958006 |
| 2188. | AC008509 | Genbank | 7574819 |
| 2189. | AC008510 | Genbank | 8190971 |
| 2190. | AC008534 | Genbank | 9558573 |
| 2191. | AC008554 | Genbank | 8886966 |
| 2192. | AC008572 | Genbank | 7341444 |
| 2193. | AC008649 | Genbank | 9690315 |
| 2194. | AC008772 | Genbank | 8844106 |
| 2195. | AC008860 | Genbank | 7549618 |
| 2196. | AC008920 | Genbank | 9625321 |
| 2197. | AC008959 | Genbank | 7582554 |
| 2198. | AC008970 | Genbank | 7839901 |
| 2199. | AC008990 | Genbank | 7209394 |
| 2200. | AC009007 | Genbank | 7527772 |
| 2201. | AC009046 | Genbank | 7229217 |
| 2202. | AC009073 | Genbank | 9211198 |
| 2203. | AC009075 | Genbank | 9211199 |
| 2204. | AC009079 | Genbank | 7340296 |
| 2205. | AC009086 | Genbank | 8122167 |
| 2206. | AC009178 | Genbank | 6642684 |
| 2207. | AC009196 | Genbank | 8927686 |
| 2208. | AC009223 | Genbank | 6042098 |
| 2209. | AC009229 | Genbank | 10716650 |
| 2210. | AC009247 | Genbank | 6539155 |
| 2211. | AC009294 | Genbank | 6272468 |
| 2212. | AC009298 | Genbank | 10716632 |
| 2213. | AC009315 | Genbank | 9945290 |
| 2214. | AC009319 | Genbank | 9558561 |
| 2215. | AC009329 | Genbank | 8810493 |
| 2216. | AC009331 | Genbank | 9789636 |
| 2217. | AC009399 | Genbank | 6682219 |
| 2218. | AC009410 | Genbank | 7243912 |
| 2219. | AC009411 | Genbank | 6587929 |
| 2220. | AC009430 | Genbank | 7131931 |
| 2221. | AC009464 | Genbank | 6492471 |
| 2222. | AC009476 | Genbank | 7243900 |
| 2223. | AC009478 | Genbank | 7770675 |
| 2224. | AC009507 | Genbank | 7243931 |
| 2225. | AC009510 | Genbank | 6449479 |
| 2226. | AC009511 | Genbank | 9558563 |
| 2227. | AC009516 | Genbank | 6437562 |
| 2228. | AC009531 | Genbank | 9802820 |
| 2229. | AC009567 | Genbank | 8072467 |
| 2230. | AC009892 | Genbank | 9937752 |
| 2231. | AC009953 | Genbank | 8954248 |
| 2232. | AC010139 | Genbank | 9581955 |
| 2233. | AC010140 | Genbank | 7622503 |
| 2234. | AC010168 | Genbank | 6855156 |
| 2235. | AC010175 | Genbank | 6139075 |
| 2236. | AC010200 | Genbank | 6492469 |
| 2237. | AC010202 | Genbank | 6598666 |
| 2238. | AC010209 | Genbank | 6648135 |
| 2239. | AC010283 | Genbank | 7328723 |
| 2240. | AC010285 | Genbank | 7651784 |
| 2241. | AC010329 | Genbank | 7328724 |
| 2242. | AC010352 | Genbank | 7109394 |
| 2243. | AC010358 | Genbank | 7019610 |
| 2244. | AC010389 | Genbank | 8844109 |
| 2245. | AC010498 | Genbank | 7243822 |
| 2246. | AC010516 | Genbank | 7289936 |
| 2247. | AC010517 | Genbank | 6223625 |
| 2248. | AC010588 | Genbank | 8886974 |
| 2249. | AC010645 | Genbank | 7670119 |
| 2250. | AC010685 | Genbank | 7534287 |
| 2251. | AC010849 | Genbank | 6524048 |
| 2252. | AC011282 | Genbank | 7534246 |
| 2253. | AC011331 | Genbank | 9929686 |
| 2254. | AC011449 | Genbank | 7839902 |
| 2255. | AC011506 | Genbank | 7630361 |
| 2256. | AC011523 | Genbank | 8778953 |
| 2257. | AC011609 | Genbank | 7114193 |
| 2258. | AC011716 | Genbank | 9857511 |
| 2259. | AC011742 | Genbank | 9887800 |
| 2260. | AC011904 | Genbank | 8051573 |
| 2261. | AC012067 | Genbank | 7656648 |
| 2262. | AC012156 | Genbank | 7363387 |
| 2263. | AC012467 | Genbank | 7363385 |
| 2264. | AC012519 | Genbank | 9558566 |
| 2265. | AC012558 | Genbank | 8886959 |
| 2266. | AC012598 | Genbank | 9864738 |
| 2267. | AC013738 | Genbank | 8567747 |
| 2268. | AC015600 | Genbank | 11128428 |
| 2269. | AC015853 | Genbank | 6721261 |
| 2270. | AC016027 | Genbank | 7958974 |
| 2271. | AC016397 | Genbank | 8567750 |
| 2272. | AC016644 | Genbank | 9937756 |
| 2273. | AC016752 | Genbank | 7637819 |
| 2274. | AC016831 | Genbank | 6539289 |
| 2275. | AC016940 | Genbank | 7381646 |
| 2276. | AC016950 | Genbank | 7114949 |
| 2277. | AC016955 | Genbank | 9864740 |
| 2278. | AC018511 | Genbank | 7715630 |
| 2279. | AC018728 | Genbank | 9743465 |
| 2280. | AC018741 | Genbank | 9665210 |
| 2281. | AC018757 | Genbank | 9625331 |
| 2282. | AC018760 | Genbank | 7019613 |
| 2283. | AC018764 | Genbank | 11128366 |
| 2284. | AC018797 | Genbank | 8747372 |
| 2285. | AC019106 | Genbank | 8570227 |
| 2286. | AC019117 | Genbank | 8748931 |
| 2287. | AC019215 | Genbank | 10048057 |
| 2288. | AC019226 | Genbank | 9858446 |
| 2289. | AC020626 | Genbank | 7656676 |
| 2290. | AC020629 | Genbank | 7656675 |
| 2291. | AC020633 | Genbank | 7658301 |
| 2292. | AC020728 | Genbank | 7770044 |
| 2293. | AC020910 | Genbank | 9558584 |
| 2294. | AC020923 | Genbank | 8886982 |
| 2295. | AC021036 | Genbank | 9885981 |
| 2296. | AC021863 | Genbank | 9887834 |
| 2297. | AC021887 | Genbank | 9581979 |
| 2298. | AC022013 | Genbank | 9858955 |
| 2299. | AC022073 | Genbank | 7363377 |
| 2300. | AC022143 | Genbank | 8099269 |
| 2301. | AC022309 | Genbank | 9945155 |
| 2302. | AC022333 | Genbank | 7341380 |
| 2303. | AC022392 | Genbank | 9885980 |
| 2304. | AC022493 | Genbank | 9558552 |
| 2305. | AC023281 | Genbank | 9558555 |
| 2306. | AC023602 | Genbank | 7276296 |
| 2307. | AC023796 | Genbank | 9558557 |
| 2308. | AC023798 | Genbank | 9910026 |
| 2309. | AC024057 | Genbank | 9887573 |
| 2310. | AC024082 | Genbank | 8810491 |
| 2311. | AC024153 | Genbank | 10190646 |
| 2312. | AC026122 | Genbank | 9929321 |
| 2313. | AC026161 | Genbank | 9858926 |
| 2314. | AC026426 | Genbank | 10086450 |
| 2315. | AC026439 | Genbank | 9690324 |
| 2316. | AC026694 | Genbank | 9625334 |
| 2317. | AC027612 | Genbank | 10179393 |
| 2318. | AC027663 | Genbank | 8698752 |
| 2319. | AC037199 | Genbank | 7527775 |
| 2320. | AC062033 | Genbank | 9858445 |
| 2321. | AC063956 | Genbank | 9910030 |
| 2322. | AC067744 | Genbank | 10198303 |
| 2323. | AC068323 | Genbank | 8698763 |
| 2324. | AC068811 | Genbank | 8810478 |
| 2325. | AC073130 | Genbank | 10140841 |
| 2326. | AC073165 | Genbank | 9857510 |
| 2327. | AD000090 | Genbank | 1905894 |
| 2328. | AE000660 | Genbank | 2358042 |
| 2329. | AE001374 | Genbank | 3845100 |
| 2330. | AF001548 | Genbank | 2104552 |
| 2331. | AF001550 | Genbank | 2335061 |
| 2332. | AF002999 | Genbank | 2529439 |
| 2333. | AF006513 | Genbank | 2645428 |
| 2334. | AF006514 | Genbank | 2645430 |
| 2335. | AF007216 | Genbank | 2281471 |
| 2336. | AF007544 | Genbank | 2970122 |
| 2337. | AF010193 | Genbank | 2252821 |
| 2338. | AF012072 | Genbank | 9967556 |
| 2339. | AF013591 | Genbank | 2338557 |
| 2340. | AF015262 | Genbank | 2897092 |
| 2341. | AF016052 | Genbank | 2394173 |
| 2342. | AF017257 | Genbank | 2736086 |
| 2343. | AF019413 | Genbank | 2347130 |
| 2344. | AF020352 | Genbank | 2655054 |
| 2345. | AF021819 | Genbank | 2460317 |
| 2346. | AF022211 | Genbank | 2465723 |
| 2347. | AF022789 | Genbank | 3220153 |
| 2348. | AF026126 | Genbank | 2828601 |
| 2349. | AF026166 | Genbank | 4090928 |
| 2350. | AF027153 | Genbank | 2739093 |
| 2351. | AF028832 | Genbank | 3287488 |
| 2352. | AF029308 | Genbank | 2564750 |
| 2353. | AF029750 | Genbank | 2587057 |
| 2354. | AF030403 | Genbank | 4103970 |
| 2355. | AF036272 | Genbank | 2921415 |
| 2356. | AF037261 | Genbank | 3004947 |
| 2357. | AF038187 | Genbank | 2795907 |
| 2358. | AF038955 | Genbank | 3329379 |
| 2359. | AF039594 | Genbank | 3366667 |
| 2360. | AF039656 | Genbank | 2773159 |
| 2361. | AF041081 | Genbank | 3660663 |
| 2362. | AF042081 | Genbank | 3337419 |
| 2363. | AF042091 | Genbank | 2829109 |
| 2364. | AF042378 | Genbank | 2801698 |
| 2365. | AF042384 | Genbank | 2828146 |
| 2366. | AF044959 | Genbank | 3348136 |
| 2367. | AF045569 | Genbank | 4226042 |
| 2368. | AF047020 | Genbank | 4204096 |
| 2369. | AF047181 | Genbank | 2909853 |
| 2370. | AF048693 | Genbank | 3170416 |
| 2371. | AF049140 | Genbank | 2947300 |
| 2372. | AF049895 | Genbank | 4126312 |
| 2373. | AF051334 | Genbank | 3126794 |
| 2374. | AF051976 | Genbank | 6138992 |
| 2375. | AF052051 | Genbank | 5668857 |
| 2376. | AF052129 | Genbank | 3360438 |
| 2377. | AF052174 | Genbank | 3360485 |
| 2378. | AF055475 | Genbank | 3511026 |
| 2379. | AF060568 | Genbank | 4138921 |
| 2380. | AF062529 | Genbank | 3978223 |
| 2381. | AF065388 | Genbank | 3152700 |
| 2382. | AF067420 | Genbank | 3201899 |
| 2383. | AF067575 | Genbank | 3789866 |
| 2384. | AF068754 | Genbank | 3283408 |
| 2385. | AF068755 | Genbank | 4321979 |
| 2386. | AF068846 | Genbank | 3201999 |
| 2387. | AF069307 | Genbank | 4884549 |
| 2388. | AF070539 | Genbank | 3387896 |
| 2389. | AF070627 | Genbank | 3283894 |
| 2390. | AF070646 | Genbank | 3283920 |
| 2391. | AF070650 | Genbank | 4454675 |
| 2392. | AF070655 | Genbank | 4454685 |
| 2393. | AF070658 | Genbank | 4454691 |
| 2394. | AF070664 | Genbank | 4454703 |
| 2395. | AF070673 | Genbank | 3978241 |
| 2396. | AF071771 | Genbank | 3243078 |
| 2397. | AF072097 | Genbank | 5725511 |
| 2398. | AF073298 | Genbank | 3641537 |
| 2399. | AF075061 | Genbank | 3377602 |
| 2400. | AF077050 | Genbank | 4689147 |
| 2401. | AF077202 | Genbank | 4679017 |
| 2402. | AF078862 | Genbank | 5531838 |
| 2403. | AF085936 | Genbank | 3483279 |
| 2404. | AF085986 | Genbank | 3483331 |
| 2405. | AF086080 | Genbank | 3483425 |
| 2406. | AF086095 | Genbank | 3483440 |
| 2407. | AF086133 | Genbank | 3483478 |
| 2408. | AF086182 | Genbank | 3483527 |
| 2409. | AF086337 | Genbank | 3483682 |
| 2410. | AF087993 | Genbank | 3523199 |
| 2411. | AF088004 | Genbank | 3523210 |
| 2412. | AF090384 | Genbank | 4226053 |
| 2413. | AF090938 | Genbank | 6690239 |
| 2414. | AF091095 | Genbank | 4165094 |
| 2415. | AF097721 | Genbank | 7211185 |
| 2416. | AF099810 | Genbank | 3800891 |
| 2417. | AF100740 | Genbank | 5138990 |
| 2418. | AF105036 | Genbank | 5353532 |
| 2419. | AF105253 | Genbank | 7532779 |
| 2420. | AF107045 | Genbank | 5006419 |
| 2421. | AF110131 | Genbank | 5565857 |
| 2422. | AF111168 | Genbank | 4186181 |
| 2423. | AF113016 | Genbank | 6642755 |
| 2424. | AF113682 | Genbank | 6855608 |
| 2425. | AF116679 | Genbank | 7959856 |
| 2426. | AF116682 | Genbank | 7959862 |
| 2427. | AF119897 | Genbank | 7770230 |
| 2428. | AF119905 | Genbank | 7770246 |
| 2429. | AF121898 | Genbank | 4235127 |
| 2430. | AF124730 | Genbank | 4262557 |
| 2431. | AF128536 | Genbank | 5305705 |
| 2432. | AF129085 | Genbank | 4928063 |
| 2433. | AF130342 | Genbank | 5430714 |
| 2434. | AF130358 | Genbank | 7547270 |
| 2435. | AF131743 | Genbank | 4406555 |
| 2436. | AF131748 | Genbank | 4406563 |
| 2437. | AF131763 | Genbank | 4406585 |
| 2438. | AF131767 | Genbank | 4406591 |
| 2439. | AF131792 | Genbank | 4406620 |
| 2440. | AF131820 | Genbank | 4406655 |
| 2441. | AF143235 | Genbank | 6449465 |
| 2442. | AF145385 | Genbank | 4929329 |
| 2443. | AF146191 | Genbank | 5678818 |
| 2444. | AF147331 | Genbank | 4761682 |
| 2445. | AF151056 | Genbank | 7106833 |
| 2446. | AF151103 | Genbank | 5758136 |
| 2447. | AF151884 | Genbank | 4929720 |
| 2448. | AF151897 | Genbank | 4929746 |
| 2449. | AF152363 | Genbank | 5669134 |
| 2450. | AF153609 | Genbank | 5231142 |
| 2451. | AF161448 | Genbank | 6841309 |
| 2452. | AF164526 | Genbank | 6815271 |
| 2453. | AF165281 | Genbank | 5734100 |
| 2454. | AF172066 | Genbank | 5733811 |
| 2455. | AF172081 | Genbank | 7528196 |
| 2456. | AF172932 | Genbank | 5726642 |
| 2457. | AF176574 | Genbank | 5762481 |
| 2458. | AF178030 | Genbank | 6539738 |
| 2459. | AF180322 | Genbank | 5881245 |
| 2460. | AF186194 | Genbank | 5923927 |
| 2461. | AF187554 | Genbank | 6653225 |
| 2462. | AF199339 | Genbank | 6708280 |
| 2463. | AF201077 | Genbank | 6456748 |
| 2464. | AF201933 | Genbank | 9295169 |
| 2465. | AF203815 | Genbank | 6979641 |
| 2466. | AF205588 | Genbank | 6531675 |
| 2467. | AF208853 | Genbank | 7582293 |
| 2468. | AF211972 | Genbank | 9885302 |
| 2469. | AF217506 | Genbank | 7688954 |
| 2470. | AF225898 | Genbank | 7021527 |
| 2471. | AF227510 | Genbank | 6984114 |
| 2472. | AF228603 | Genbank | 6984179 |
| 2473. | AF233395 | Genbank | 7243746 |
| 2474. | AF240629 | Genbank | 7263185 |
| 2475. | AF241734 | Genbank | 7417369 |
| 2476. | AF242380 | Genbank | 8248261 |
| 2477. | AF247704 | Genbank | 9963969 |
| 2478. | AF250324 | Genbank | 9930130 |
| 2479. | AF252279 | Genbank | 8926171 |
| 2480. | AF265205 | Genbank | 9864061 |
| 2481. | AF276886 | Genbank | 9502246 |
| 2482. | AF278605 | Genbank | 9966507 |
| 2483. | AF283772 | Genbank | 10281741 |
| 2484. | AF285120 | Genbank | 9858830 |
| 2485. | AJ000882 | Genbank | 2924310 |
| 2486. | AJ006837 | Genbank | 3282045 |
| 2487. | AJ010597 | Genbank | 3559873 |
| 2488. | AJ010598 | Genbank | 3559851 |
| 2489. | AJ132695 | Genbank | 8574037 |
| 2490. | AJ223812 | Genbank | 2894518 |
| 2491. | AJ239321 | Genbank | 5931819 |
| 2492. | AJ239323 | Genbank | 7018303 |
| 2493. | AJ243211 | Genbank | 6624919 |
| 2494. | AJ243213 | Genbank | 6900061 |
| 2495. | AJ271735 | Genbank | 8979788 |
| 2496. | AJ276674 | Genbank | 7329720 |
| 2497. | AJ277892 | Genbank | 8249466 |
| 2498. | AK000023 | Genbank | 7019837 |
| 2499. | AK000058 | Genbank | 7019896 |
| 2500. | AK000078 | Genbank | 7019932 |
| 2501. | AK000168 | Genbank | 7020079 |
| 2502. | AK000285 | Genbank | 7020262 |
| 2503. | AK000348 | Genbank | 7020373 |
| 2504. | AK000445 | Genbank | 7020539 |
| 2505. | AK000500 | Genbank | 7020630 |
| 2506. | AK000560 | Genbank | 7020738 |
| 2507. | AK000796 | Genbank | 7021099 |
| 2508. | AK001029 | Genbank | 7022050 |
| 2509. | AK001061 | Genbank | 7022095 |
| 2510. | AK001152 | Genbank | 7022225 |
| 2511. | AK001282 | Genbank | 7022438 |
| 2512. | AK001313 | Genbank | 7022490 |
| 2513. | AK001569 | Genbank | 7022902 |
| 2514. | AK001635 | Genbank | 7023008 |
| 2515. | AK001680 | Genbank | 7023088 |
| 2516. | AK001718 | Genbank | 7023153 |
| 2517. | AK002161 | Genbank | 7023871 |
| 2518. | AK002191 | Genbank | 7023916 |
| 2519. | AL008582 | Genbank | 5360981 |
| 2520. | AL008635 | Genbank | 3183882 |
| 2521. | AL008637 | Genbank | 3136000 |
| 2522. | AL008639 | Genbank | 6599068 |
| 2523. | AL008718 | Genbank | 6006479 |
| 2524. | AL008720 | Genbank | 2956679 |
| 2525. | AL008725 | Genbank | 2791551 |
| 2526. | AL008730 | Genbank | 2842415 |
| 2527. | AL020993 | Genbank | 3980107 |
| 2528. | AL020995 | Genbank | 10862821 |
| 2529. | AL021368 | Genbank | 3080468 |
| 2530. | AL021408 | Genbank | 3123551 |
| 2531. | AL021451 | Genbank | 2815069 |
| 2532. | AL021546 | Genbank | 2826890 |
| 2533. | AL021807 | Genbank | 9367202 |
| 2534. | AL021938 | Genbank | 3242170 |
| 2535. | AL022100 | Genbank | 5881340 |
| 2536. | AL022101 | Genbank | 3171895 |
| 2537. | AL022319 | Genbank | 4884062 |
| 2538. | AL022336 | Genbank | 3336981 |
| 2539. | AL022394 | Genbank | 9581758 |
| 2540. | AL022395 | Genbank | 4468287 |
| 2541. | AL022725 | Genbank | 5679748 |
| 2542. | AL022726 | Genbank | 3676217 |
| 2543. | AL023284 | Genbank | 3355875 |
| 2544. | AL023580 | Genbank | 3702133 |
| 2545. | AL023656 | Genbank | 5679746 |
| 2546. | AL023773 | Genbank | 3449129 |
| 2547. | AL023798 | Genbank | 3550031 |
| 2548. | AL023800 | Genbank | 3288055 |
| 2549. | AL023806 | Genbank | 3288443 |
| 2550. | AL023878 | Genbank | 3947686 |
| 2551. | AL024498 | Genbank | 5924006 |
| 2552. | AL024508 | Genbank | 3445456 |
| 2553. | AL030996 | Genbank | 3688349 |
| 2554. | AL031114 | Genbank | 3618151 |
| 2555. | AL031281 | Genbank | 4826460 |
| 2556. | AL031293 | Genbank | 4140361 |
| 2557. | AL031347 | Genbank | 4160208 |
| 2558. | AL031428 | Genbank | 5931719 |
| 2559. | AL031432 | Genbank | 4375969 |
| 2560. | AL031542 | Genbank | 4007105 |
| 2561. | AL031594 | Genbank | 5050980 |
| 2562. | AL031602 | Genbank | 6729581 |
| 2563. | AL031655 | Genbank | 5360979 |
| 2564. | AL031665 | Genbank | 4826504 |
| 2565. | AL031666 | Genbank | 10198598 |
| 2566. | AL031667 | Genbank | 10198629 |
| 2567. | AL031668 | Genbank | 10198603 |
| 2568. | AL031673 | Genbank | 10198607 |
| 2569. | AL031679 | Genbank | 4007546 |
| 2570. | AL031681 | Genbank | 10198606 |
| 2571. | AL031683 | Genbank | 9368419 |
| 2572. | AL031767 | Genbank | 6969156 |
| 2573. | AL031770 | Genbank | 5050946 |
| 2574. | AL031776 | Genbank | 6468548 |
| 2575. | AL031780 | Genbank | 4140349 |
| 2576. | AL031885 | Genbank | 4464237 |
| 2577. | AL031904 | Genbank | 3766261 |
| 2578. | AL031965 | Genbank | 4582124 |
| 2579. | AL032821 | Genbank | 4572584 |
| 2580. | AL033384 | Genbank | 4455570 |
| 2581. | AL033527 | Genbank | 6456853 |
| 2582. | AL033531 | Genbank | 6807582 |
| 2583. | AL033533 | Genbank | 4468336 |
| 2584. | AL033547 | Genbank | 4884064 |
| 2585. | AL034349 | Genbank | 4680410 |
| 2586. | AL034429 | Genbank | 4376012 |
| 2587. | AL034553 | Genbank | 5419653 |
| 2588. | AL034558 | Genbank | 4493878 |
| 2589. | AL035070 | Genbank | 4455405 |
| 2590. | AL035089 | Genbank | 6742111 |
| 2591. | AL035409 | Genbank | 5514663 |
| 2592. | AL035410 | Genbank | 4775617 |
| 2593. | AL035419 | Genbank | 10198625 |
| 2594. | AL035448 | Genbank | 5830352 |
| 2595. | AL035533 | Genbank | 4455648 |
| 2596. | AL035541 | Genbank | 10198628 |
| 2597. | AL035652 | Genbank | 10242468 |
| 2598. | AL035663 | Genbank | 6018761 |
| 2599. | AL035682 | Genbank | 5596686 |
| 2600. | AL035688 | Genbank | 4902610 |
| 2601. | AL035697 | Genbank | 5924005 |
| 2602. | AL049229 | Genbank | 4499961 |
| 2603. | AL049265 | Genbank | 4500013 |
| 2604. | AL049266 | Genbank | 4500015 |
| 2605. | AL049338 | Genbank | 4500120 |
| 2606. | AL049367 | Genbank | 4500158 |
| 2607. | AL049415 | Genbank | 4500196 |
| 2608. | AL049471 | Genbank | 4500266 |
| 2609. | AL049564 | Genbank | 4902757 |
| 2610. | AL049575 | Genbank | 5304892 |
| 2611. | AL049595 | Genbank | 4775657 |
| 2612. | AL049597 | Genbank | 9663352 |
| 2613. | AL049610 | Genbank | 5679448 |
| 2614. | AL049649 | Genbank | 9795225 |
| 2615. | AL049699 | Genbank | 5419832 |
| 2616. | AL049715 | Genbank | 9581791 |
| 2617. | AL049735 | Genbank | 5596978 |
| 2618. | AL049742 | Genbank | 5419783 |
| 2619. | AL049744 | Genbank | 6782357 |
| 2620. | AL049762 | Genbank | 8218084 |
| 2621. | AL049775 | Genbank | 5019423 |
| 2622. | AL049776 | Genbank | 6681704 |
| 2623. | AL049777 | Genbank | 7838309 |
| 2624. | AL049781 | Genbank | 6425538 |
| 2625. | AL049794 | Genbank | 10280528 |
| 2626. | AL049795 | Genbank | 6010175 |
| 2627. | AL049820 | Genbank | 8247261 |
| 2628. | AL049830 | Genbank | 6729561 |
| 2629. | AL049835 | Genbank | 5708093 |
| 2630. | AL049870 | Genbank | 7248268 |
| 2631. | AL049875 | Genbank | 5524775 |
| 2632. | AL049911 | Genbank | 6114591 |
| 2633. | AL049925 | Genbank | 4884172 |
| 2634. | AL049929 | Genbank | 4884174 |
| 2635. | AL049951 | Genbank | 4884198 |
| 2636. | AL049969 | Genbank | 4884218 |
| 2637. | AL050268 | Genbank | 4886442 |
| 2638. | AL050307 | Genbank | 6562569 |
| 2639. | AL050310 | Genbank | 5459289 |
| 2640. | AL050319 | Genbank | 6015564 |
| 2641. | AL050339 | Genbank | 9795229 |
| 2642. | AL050343 | Genbank | 6137008 |
| 2643. | AL050349 | Genbank | 8655549 |
| 2644. | AL050363 | Genbank | 4914597 |
| 2645. | AL050376 | Genbank | 4914609 |
| 2646. | AL078463 | Genbank | 5777575 |
| 2647. | AL078585 | Genbank | 6010190 |
| 2648. | AL078588 | Genbank | 5823985 |
| 2649. | AL078591 | Genbank | 6562085 |
| 2650. | AL078593 | Genbank | 5650629 |
| 2651. | AL078599 | Genbank | 5804908 |
| 2652. | AL078614 | Genbank | 6425557 |
| 2653. | AL078639 | Genbank | 6523669 |
| 2654. | AL078645 | Genbank | 8218091 |
| 2655. | AL079295 | Genbank | 5102607 |
| 2656. | AL079341 | Genbank | 8655533 |
| 2657. | AL079343 | Genbank | 7619709 |
| 2658. | AL080209 | Genbank | 5262698 |
| 2659. | AL080241 | Genbank | 5730204 |
| 2660. | AL080243 | Genbank | 5870622 |
| 2661. | AL080272 | Genbank | 5725275 |
| 2662. | AL080284 | Genbank | 8546828 |
| 2663. | AL080285 | Genbank | 5566556 |
| 2664. | AL096678 | Genbank | 5912558 |
| 2665. | AL096703 | Genbank | 5791541 |
| 2666. | AL096711 | Genbank | 5763763 |
| 2667. | AL096769 | Genbank | 5596937 |
| 2668. | AL096771 | Genbank | 6165369 |
| 2669. | AL096801 | Genbank | 6478163 |
| 2670. | AL096862 | Genbank | 6706906 |
| 2671. | AL109614 | Genbank | 8745190 |
| 2672. | AL109621 | Genbank | 5918014 |
| 2673. | AL109653 | Genbank | 6318116 |
| 2674. | AL109658 | Genbank | 7161806 |
| 2675. | AL109662 | Genbank | 8176899 |
| 2676. | AL109759 | Genbank | 8176900 |
| 2677. | AL109766 | Genbank | 7159614 |
| 2678. | AL109839 | Genbank | 6634462 |
| 2679. | AL109925 | Genbank | 7105935 |
| 2680. | AL109928 | Genbank | 7981303 |
| 2681. | AL109939 | Genbank | 6523730 |
| 2682. | AL109941 | Genbank | 6822193 |
| 2683. | AL109944 | Genbank | 8218116 |
| 2684. | AL110122 | Genbank | 5805143 |
| 2685. | AL110180 | Genbank | 5817090 |
| 2686. | AL110194 | Genbank | 5817111 |
| 2687. | AL110197 | Genbank | 5817115 |
| 2688. | AL117336 | Genbank | 9581769 |
| 2689. | AL117340 | Genbank | 6002147 |
| 2690. | AL117345 | Genbank | 8218101 |
| 2691. | AL117347 | Genbank | 6143597 |
| 2692. | AL117354 | Genbank | 6822199 |
| 2693. | AL117355 | Genbank | 6469348 |
| 2694. | AL117356 | Genbank | 6469349 |
| 2695. | AL117383 | Genbank | 7406730 |
| 2696. | AL117406 | Genbank | 5911991 |
| 2697. | AL117667 | Genbank | 9187469 |
| 2698. | AL117693 | Genbank | 6967147 |
| 2699. | AL118497 | Genbank | 6453387 |
| 2700. | AL118510 | Genbank | 6572394 |
| 2701. | AL118519 | Genbank | 9187342 |
| 2702. | AL118523 | Genbank | 9795217 |
| 2703. | AL121579 | Genbank | 7271127 |
| 2704. | AL121580 | Genbank | 6456854 |
| 2705. | AL121582 | Genbank | 8218070 |
| 2706. | AL121583 | Genbank | 9588417 |
| 2707. | AL121584 | Genbank | 8218072 |
| 2708. | AL121601 | Genbank | 7159760 |
| 2709. | AL121652 | Genbank | 7159615 |
| 2710. | AL121714 | Genbank | 9369303 |
| 2711. | AL121716 | Genbank | 7159797 |
| 2712. | AL121724 | Genbank | 7378737 |
| 2713. | AL121755 | Genbank | 7406634 |
| 2714. | AL121757 | Genbank | 6114793 |
| 2715. | AL121766 | Genbank | 6433829 |
| 2716. | AL121767 | Genbank | 10944719 |
| 2717. | AL121790 | Genbank | 8919824 |
| 2718. | AL121809 | Genbank | 8018146 |
| 2719. | AL121820 | Genbank | 8176917 |
| 2720. | AL121821 | Genbank | 7799025 |
| 2721. | AL121838 | Genbank | 8217875 |
| 2722. | AL121852 | Genbank | 8176919 |
| 2723. | AL121870 | Genbank | 7161190 |
| 2724. | AL121893 | Genbank | 9187340 |
| 2725. | AL121914 | Genbank | 9864672 |
| 2726. | AL121927 | Genbank | 8977863 |
| 2727. | AL121929 | Genbank | 8218073 |
| 2728. | AL121931 | Genbank | 7406486 |
| 2729. | AL121947 | Genbank | 9501323 |
| 2730. | AL121958 | Genbank | 8247268 |
| 2731. | AL121961 | Genbank | 8894647 |
| 2732. | AL121967 | Genbank | 9944262 |
| 2733. | AL121968 | Genbank | 9581789 |
| 2734. | AL121973 | Genbank | 6434691 |
| 2735. | AL121989 | Genbank | 8919105 |
| 2736. | AL122023 | Genbank | 8217876 |
| 2737. | AL122057 | Genbank | 7710965 |
| 2738. | AL122125 | Genbank | 8574298 |
| 2739. | AL132639 | Genbank | 7708219 |
| 2740. | AL132655 | Genbank | 9187351 |
| 2741. | AL132665 | Genbank | 6137021 |
| 2742. | AL132777 | Genbank | 6562026 |
| 2743. | AL132794 | Genbank | 9944264 |
| 2744. | AL132826 | Genbank | 10198649 |
| 2745. | AL133009 | Genbank | 6453414 |
| 2746. | AL133014 | Genbank | 6453427 |
| 2747. | AL133173 | Genbank | 8217417 |
| 2748. | AL133227 | Genbank | 9187135 |
| 2749. | AL133228 | Genbank | 8217426 |
| 2750. | AL133230 | Genbank | 8574103 |
| 2751. | AL133240 | Genbank | 8346743 |
| 2752. | AL133241 | Genbank | 7630038 |
| 2753. | AL133243 | Genbank | 6491713 |
| 2754. | AL133247 | Genbank | 6491719 |
| 2755. | AL133255 | Genbank | 8894156 |
| 2756. | AL133282 | Genbank | 8246854 |
| 2757. | AL133283 | Genbank | 7267032 |
| 2758. | AL133284 | Genbank | 8217432 |
| 2759. | AL133289 | Genbank | 6729371 |
| 2760. | AL133295 | Genbank | 8217436 |
| 2761. | AL133304 | Genbank | 6624624 |
| 2762. | AL133367 | Genbank | 7708222 |
| 2763. | AL133380 | Genbank | 8217443 |
| 2764. | AL133404 | Genbank | 6706820 |
| 2765. | AL133415 | Genbank | 7160477 |
| 2766. | AL133419 | Genbank | 7767917 |
| 2767. | AL133449 | Genbank | 10432563 |
| 2768. | AL133453 | Genbank | 7009594 |
| 2769. | AL133454 | Genbank | 8919826 |
| 2770. | AL133479 | Genbank | 8919196 |
| 2771. | AL133500 | Genbank | 8248740 |
| 2772. | AL133515 | Genbank | 8248647 |
| 2773. | AL133544 | Genbank | 7406719 |
| 2774. | AL133655 | Genbank | 6599245 |
| 2775. | AL135744 | Genbank | 6682291 |
| 2776. | AL135749 | Genbank | 7768193 |
| 2777. | AL135786 | Genbank | 8894169 |
| 2778. | AL135787 | Genbank | 9588110 |
| 2779. | AL135788 | Genbank | 8894170 |
| 2780. | AL135901 | Genbank | 10185402 |
| 2781. | AL135928 | Genbank | 9863460 |
| 2782. | AL136000 | Genbank | 7940101 |
| 2783. | AL136090 | Genbank | 9662903 |
| 2784. | AL136094 | Genbank | 9187149 |
| 2785. | AL136097 | Genbank | 7799632 |
| 2786. | AL136131 | Genbank | 10045276 |
| 2787. | AL136167 | Genbank | 7159407 |
| 2788. | AL136231 | Genbank | 9662908 |
| 2789. | AL136321 | Genbank | 7801524 |
| 2790. | AL136370 | Genbank | 8018015 |
| 2791. | AL136374 | Genbank | 8919204 |
| 2792. | AL136382 | Genbank | 8452462 |
| 2793. | AL136441 | Genbank | 8217480 |
| 2794. | AL136504 | Genbank | 7018294 |
| 2795. | AL136985 | Genbank | 9542710 |
| 2796. | AL137000 | Genbank | 9944121 |
| 2797. | AL137020 | Genbank | 10086020 |
| 2798. | AL137022 | Genbank | 8217493 |
| 2799. | AL137072 | Genbank | 9650521 |
| 2800. | AL137129 | Genbank | 7009596 |
| 2801. | AL137159 | Genbank | 9796041 |
| 2802. | AL137164 | Genbank | 7019736 |
| 2803. | AL137221 | Genbank | 9650522 |
| 2804. | AL137438 | Genbank | 6807997 |
| 2805. | AL137440 | Genbank | 6808001 |
| 2806. | AL137628 | Genbank | 6808426 |
| 2807. | AL137784 | Genbank | 9863487 |
| 2808. | AL137818 | Genbank | 7009598 |
| 2809. | AL137861 | Genbank | 9187172 |
| 2810. | AL138783 | Genbank | 9712551 |
| 2811. | AL138954 | Genbank | 8978058 |
| 2812. | AL138976 | Genbank | 7799028 |
| 2813. | AL139120 | Genbank | 7739105 |
| 2814. | AL139182 | Genbank | 9581567 |
| 2815. | AL139186 | Genbank | 9856677 |
| 2816. | AL139195 | Genbank | 8218586 |
| 2817. | AL139233 | Genbank | 9944132 |
| 2818. | AL139295 | Genbank | 8439264 |
| 2819. | AL139316 | Genbank | 8217940 |
| 2820. | AL139317 | Genbank | 8217941 |
| 2821. | AL139342 | Genbank | 9796485 |
| 2822. | AL139353 | Genbank | 6996091 |
| 2823. | AL139379 | Genbank | 8894228 |
| 2824. | AL139806 | Genbank | 9588501 |
| 2825. | AL139807 | Genbank | 8217582 |
| 2826. | AL157360 | Genbank | 7899160 |
| 2827. | AL157398 | Genbank | 10045326 |
| 2828. | AL157448 | Genbank | 7018462 |
| 2829. | AL157488 | Genbank | 7018533 |
| 2830. | AL157792 | Genbank | 7327842 |
| 2831. | AL157826 | Genbank | 8918980 |
| 2832. | AL157837 | Genbank | 9801319 |
| 2833. | AL157879 | Genbank | 8217606 |
| 2834. | AL157903 | Genbank | 9967449 |
| 2835. | AL158040 | Genbank | 8745067 |
| 2836. | AL158111 | Genbank | 7288041 |
| 2837. | AL158157 | Genbank | 9662926 |
| 2838. | AL158206 | Genbank | 8977646 |
| 2839. | AL158832 | Genbank | 9944141 |
| 2840. | AL160161 | Genbank | 10045353 |
| 2841. | AL160231 | Genbank | 7768126 |
| 2842. | AL161426 | Genbank | 9369063 |
| 2843. | AL161449 | Genbank | 8894259 |
| 2844. | AL161725 | Genbank | 10045359 |
| 2845. | AL161756 | Genbank | 9187474 |
| 2846. | AL161951 | Genbank | 7327996 |
| 2847. | AL161952 | Genbank | 7328002 |
| 2848. | AL162047 | Genbank | 7328089 |
| 2849. | AL162151 | Genbank | 8655500 |
| 2850. | AL162390 | Genbank | 8894273 |
| 2851. | AL162718 | Genbank | 9944151 |
| 2852. | AL163203 | Genbank | 7717244 |
| 2853. | AL163204 | Genbank | 7717247 |
| 2854. | AL163249 | Genbank | 7717307 |
| 2855. | AL163279 | Genbank | 7717362 |
| 2856. | AL163284 | Genbank | 7717380 |
| 2857. | AL163302 | Genbank | 7717445 |
| 2858. | AL163533 | Genbank | 9800736 |
| 2859. | AL352979 | Genbank | 8546789 |
| 2860. | AL352984 | Genbank | 8248727 |
| 2861. | AL353738 | Genbank | 9581619 |
| 2862. | AL353746 | Genbank | 10045383 |
| 2863. | AL353753 | Genbank | 9988303 |
| 2864. | AL353759 | Genbank | 8745068 |
| 2865. | AL355072 | Genbank | 8017357 |
| 2866. | AL355272 | Genbank | 9187242 |
| 2867. | AL355520 | Genbank | 9863726 |
| 2868. | AL355708 | Genbank | 7799100 |
| 2869. | AL355871 | Genbank | 9407857 |
| 2870. | AL356276 | Genbank | 9407868 |
| 2871. | AL356388 | Genbank | 10045425 |
| 2872. | AL357095 | Genbank | 8655502 |
| 2873. | AL357153 | Genbank | 8919854 |
| 2874. | AL358337 | Genbank | 8346767 |
| 2875. | AL359457 | Genbank | 9944181 |
| 2876. | AL359951 | Genbank | 9187972 |
| 2877. | AL389883 | Genbank | 9864270 |
| 2878. | AP000009 | Genbank | 4666255 |
| 2879. | AP000021 | Genbank | 4666265 |
| 2880. | AP000022 | Genbank | 4666266 |
| 2881. | AP000030 | Genbank | 3132340 |
| 2882. | AP000072 | Genbank | 4579993 |
| 2883. | AP000081 | Genbank | 4580002 |
| 2884. | AP000084 | Genbank | 4730825 |
| 2885. | AP000097 | Genbank | 4730871 |
| 2886. | AP000203 | Genbank | 4827141 |
| 2887. | AP000345 | Genbank | 5103008 |
| 2888. | AP000403 | Genbank | 5811596 |
| 2889. | AP000426 | Genbank | 8698836 |
| 2890. | AP000476 | Genbank | 5922638 |
| 2891. | AP000495 | Genbank | 5926677 |
| 2892. | AP000523 | Genbank | 5931501 |
| 2893. | AP000525 | Genbank | 5931503 |
| 2894. | AP000527 | Genbank | 5931505 |
| 2895. | AP000533 | Genbank | 5931511 |
| 2896. | AP000534 | Genbank | 5931512 |
| 2897. | AP000561 | Genbank | 6015478 |
| 2898. | AP000693 | Genbank | 6693637 |
| 2899. | AP000855 | Genbank | 6683139 |
| 2900. | AP000957 | Genbank | 7077200 |
| 2901. | AP000961 | Genbank | 6705921 |
| 2902. | AP000962 | Genbank | 6942330 |
| 2903. | AP000964 | Genbank | 6778730 |
| 2904. | AP001037 | Genbank | 6693587 |
| 2905. | AP001053 | Genbank | 6693603 |
| 2906. | AP001116 | Genbank | 6899855 |
| 2907. | AP001136 | Genbank | 6970360 |
| 2908. | AP001207 | Genbank | 8698839 |
| 2909. | AP001213 | Genbank | 10121098 |
| 2910. | AP001214 | Genbank | 10121099 |
| 2911. | AP001230 | Genbank | 10121135 |
| 2912. | AP001251 | Genbank | 7077180 |
| 2913. | AP001252 | Genbank | 7077183 |
| 2914. | AP001253 | Genbank | 7077185 |
| 2915. | AP001340 | Genbank | 7209828 |
| 2916. | AP001341 | Genbank | 7209829 |
| 2917. | AP001343 | Genbank | 7209831 |
| 2918. | AP001625 | Genbank | 7670579 |
| 2919. | AP001626 | Genbank | 7670580 |
| 2920. | AP001630 | Genbank | 7670584 |
| 2921. | AP001820 | Genbank | 7594885 |
| 2922. | AP002028 | Genbank | 9293863 |
| 2923. | AX011717 | Genbank | 9998241 |
| 2924. | AX011750 | Genbank | 9998274 |
| 2925. | AX012177 | Genbank | 9998280 |
| 2926. | AX013082 | Genbank | 10040248 |
| 2927. | AX013695 | Genbank | 10040405 |
| 2928. | AX013712 | Genbank | 10040422 |
| 2929. | AX013777 | Genbank | 10040487 |
| 2930. | AX014146 | Genbank | 10040593 |
| 2931. | AX014313 | Genbank | 10040667 |
| 2932. | AX014316 | Genbank | 10040670 |
| 2933. | AX014868 | Genbank | 10041135 |
| 2934. | AX014883 | Genbank | 10041150 |
| 2935. | AX014910 | Genbank | 10041177 |
| 2936. | AX015525 | Genbank | 10041407 |
| 2937. | AX017269 | Genbank | 10042187 |
| 2938. | AX017296 | Genbank | 10042214 |
| 2939. | AX017496 | Genbank | 10042293 |
| 2940. | AX023978 | Genbank | 10184295 |
| 2941. | D00763 | Genbank | 220029 |
| 2942. | D00860 | Genbank | 220019 |
| 2943. | D13119 | Genbank | 285909 |
| 2944. | D13665 | Genbank | 393318 |
| 2945. | D14658 | Genbank | 285940 |
| 2946. | D14661 | Genbank | 285946 |
| 2947. | D14696 | Genbank | 285962 |
| 2948. | D21063 | Genbank | 434752 |
| 2949. | D21092 | Genbank | 540512 |
| 2950. | D21260 | Genbank | 434760 |
| 2951. | D25218 | Genbank | 434778 |
| 2952. | D28118 | Genbank | 529640 |
| 2953. | D30036 | Genbank | 1060902 |
| 2954. | D31767 | Genbank | 505091 |
| 2955. | D38551 | Genbank | 1531549 |
| 2956. | D45132 | Genbank | 1405347 |
| 2957. | D45370 | Genbank | 871884 |
| 2958. | D45915 | Genbank | 1483130 |
| 2959. | D45917 | Genbank | 666015 |
| 2960. | D49400 | Genbank | 1395161 |
| 2961. | D49547 | Genbank | 710654 |
| 2962. | D50916 | Genbank | 1469174 |
| 2963. | D55654 | Genbank | 1255603 |
| 2964. | D63477 | Genbank | 1469867 |
| 2965. | D67031 | Genbank | 2696053 |
| 2966. | D79993 | Genbank | 1136401 |
| 2967. | D84907 | Genbank | 1596053 |
| 2968. | D86960 | Genbank | 1503993 |
| 2969. | D87003 | Genbank | 2114242 |
| 2970. | D87005 | Genbank | 2114244 |
| 2971. | D87018 | Genbank | 2114280 |
| 2972. | D87450 | Genbank | 1665788 |
| 2973. | D87666 | Genbank | 1620016 |
| 2974. | D88546 | Genbank | 3123717 |
| 2975. | E01816 | Genbank | 2170068 |
| 2976. | E01915 | Genbank | 2170164 |
| 2977. | E07218 | Genbank | 2175359 |
| 2978. | E13299 | Genbank | 3252104 |
| 2979. | G15797 | Genbank | 1161686 |
| 2980. | G17579 | Genbank | 1215005 |
| 2981. | G50025 | Genbank | 5221202 |
| 2982. | G51725 | Genbank | 5223052 |
| 2983. | G55393 | Genbank | 6120712 |
| 2984. | G55734 | Genbank | 6120903 |
| 2985. | G56177 | Genbank | 6121346 |
| 2986. | G57377 | Genbank | 6122546 |
| 2987. | G57404 | Genbank | 6122573 |
| 2988. | G57826 | Genbank | 6122995 |
| 2989. | G58374 | Genbank | 6123693 |
| 2990. | G58708 | Genbank | 6124027 |
| 2991. | G62532 | Genbank | 6599651 |
| 2992. | G62837 | Genbank | 6599956 |
| 2993. | G63211 | Genbank | 6600180 |
| 2994. | G63650 | Genbank | 6600619 |
| 2995. | J00200 | Genbank | 188411 |
| 2996. | J03040 | Genbank | 338312 |
| 2997. | L05092 | Genbank | 388031 |
| 2998. | L11910 | Genbank | 292420 |
| 2999. | L12136 | Genbank | 181536 |
| 3000. | L12468 | Genbank | 347892 |
| 3001. | L13460 | Genbank | 499855 |
| 3002. | L17697 | Genbank | 308181 |
| 3003. | L19739 | Genbank | 431318 |
| 3004. | L28809 | Genbank | 454151 |
| 3005. | L34789 | Genbank | 514934 |
| 3006. | L38941 | Genbank | 1008855 |
| 3007. | L38951 | Genbank | 893287 |
| 3008. | L47168 | Genbank | 976207 |
| 3009. | L47234 | Genbank | 1196459 |
| 3010. | L77701 | Genbank | 1280205 |
| 3011. | M11146 | Genbank | 182504 |
| 3012. | M11147 | Genbank | 182513 |
| 3013. | M11167 | Genbank | 337381 |
| 3014. | M11560 | Genbank | 178350 |
| 3015. | M11718 | Genbank | 180912 |
| 3016. | M11725 | Genbank | 181067 |
| 3017. | M12267 | Genbank | 189328 |
| 3018. | M13082 | Genbank | 332048 |
| 3019. | M15007 | Genbank | 339173 |
| 3020. | M16985 | Genbank | 187282 |
| 3021. | M18366 | Genbank | 179131 |
| 3022. | M22430 | Genbank | 190888 |
| 3023. | M22489 | Genbank | 179501 |
| 3024. | M22538 | Genbank | 986883 |
| 3025. | M22865 | Genbank | 181226 |
| 3026. | M22918 | Genbank | 189019 |
| 3027. | M23492 | Genbank | 187005 |
| 3028. | M23613 | Genbank | 189271 |
| 3029. | M25246 | Genbank | 340233 |
| 3030. | M26481 | Genbank | 619789 |
| 3031. | M26663 | Genbank | 618463 |
| 3032. | M29366 | Genbank | 181979 |
| 3033. | M33600 | Genbank | 188240 |
| 3034. | M34840 | Genbank | 189620 |
| 3035. | M37104 | Genbank | 179274 |
| 3036. | M37583 | Genbank | 184059 |
| 3037. | M65181 | Genbank | 177128 |
| 3038. | M73554 | Genbank | 179364 |
| 3039. | M81757 | Genbank | 337732 |
| 3040. | M86400 | Genbank | 189952 |
| 3041. | M87790 | Genbank | 185363 |
| 3042. | M88108 | Genbank | 189499 |
| 3043. | M90058 | Genbank | 338031 |
| 3044. | M93651 | Genbank | 338038 |
| 3045. | M94859 | Genbank | 179831 |
| 3046. | M95724 | Genbank | 180246 |
| 3047. | S73498 | Genbank | 688010 |
| 3048. | S75421 | Genbank | 913537 |
| 3049. | S82496 | Genbank | 1699161 |
| 3050. | U00115 | Genbank | 392426 |
| 3051. | U07550 | Genbank | 469170 |
| 3052. | U07919 | Genbank | 995897 |
| 3053. | U10248 | Genbank | 984280 |
| 3054. | U12404 | Genbank | 531170 |
| 3055. | U12465 | Genbank | 562073 |
| 3056. | U13369 | Genbank | 555853 |
| 3057. | U13616 | Genbank | 608024 |
| 3058. | U14972 | Genbank | 550024 |
| 3059. | U15008 | Genbank | 600747 |
| 3060. | U16738 | Genbank | 608516 |
| 3061. | U18300 | Genbank | 1536965 |
| 3062. | U19759 | Genbank | 2745950 |
| 3063. | U24223 | Genbank | 1215670 |
| 3064. | U27460 | Genbank | 881393 |
| 3065. | U28416 | Genbank | 902884 |
| 3066. | U29669 | Genbank | 1002601 |
| 3067. | U32944 | Genbank | 1209060 |
| 3068. | U38784 | Genbank | 1574947 |
| 3069. | U38817 | Genbank | 1401054 |
| 3070. | U41384 | Genbank | 1145886 |
| 3071. | U41514 | Genbank | 1136284 |
| 3072. | U41515 | Genbank | 1209723 |
| 3073. | U41668 | Genbank | 1477481 |
| 3074. | U43399 | Genbank | 1184724 |
| 3075. | U46689 | Genbank | 1870243 |
| 3076. | U51244 | Genbank | 1255786 |
| 3077. | U53930 | Genbank | 4204850 |
| 3078. | U61397 | Genbank | 1518693 |
| 3079. | U64898 | Genbank | 2897866 |
| 3080. | U66661 | Genbank | 1857125 |
| 3081. | U68105 | Genbank | 1562509 |
| 3082. | U68385 | Genbank | 1679673 |
| 3083. | U70439 | Genbank | 1698782 |
| 3084. | U73024 | Genbank | 1613887 |
| 3085. | U73646 | Genbank | 2281067 |
| 3086. | U79251 | Genbank | 1710199 |
| 3087. | U80735 | Genbank | 2565045 |
| 3088. | U82670 | Genbank | 7274891 |
| 3089. | U90028 | Genbank | 2745975 |
| 3090. | U90426 | Genbank | 1905997 |
| 3091. | U91321 | Genbank | 2951946 |
| 3092. | U91322 | Genbank | 2344876 |
| 3093. | U91326 | Genbank | 1931583 |
| 3094. | U94728 | Genbank | 2055423 |
| 3095. | V00507 | Genbank | 30774 |
| 3096. | X05014 | Genbank | 29599 |
| 3097. | X05236 | Genbank | 28596 |
| 3098. | X06747 | Genbank | 36101 |
| 3099. | X12791 | Genbank | 36112 |
| 3100. | X15341 | Genbank | 1197215 |
| 3101. | X16064 | Genbank | 37495 |
| 3102. | X16560 | Genbank | 30154 |
| 3103. | X52851 | Genbank | 30167 |
| 3104. | X54304 | Genbank | 34755 |
| 3105. | X54941 | Genbank | 29976 |
| 3106. | X61123 | Genbank | 29508 |
| 3107. | X63469 | Genbank | 37069 |
| 3108. | X66290 | Genbank | 28642 |
| 3109. | X67336 | Genbank | 871300 |
| 3110. | X69908 | Genbank | 38431 |
| 3111. | X70326 | Genbank | 38434 |
| 3112. | X76061 | Genbank | 416030 |
| 3113. | X77748 | Genbank | 1171563 |
| 3114. | X80910 | Genbank | 531475 |
| 3115. | X87344 | Genbank | 1054740 |
| 3116. | X91648 | Genbank | 1495073 |
| 3117. | Y10196 | Genbank | 1834504 |
| 3118. | Z11692 | Genbank | 31107 |
| 3119. | Z12006 | Genbank | 28884 |
| 3120. | Z12011 | Genbank | 28888 |
| 3121. | Z21507 | Genbank | 38521 |
| 3122. | Z31696 | Genbank | 479156 |
| 3123. | Z36836 | Genbank | 533950 |
| 3124. | Z50022 | Genbank | 1107702 |
| 3125. | Z51141 | Genbank | 1232441 |
| 3126. | Z68226 | Genbank | 1122887 |
| 3127. | Z70051 | Genbank | 1223746 |
| 3128. | Z74619 | Genbank | 1405894 |
| 3129. | Z75889 | Genbank | 1430780 |
| 3130. | Z76735 | Genbank | 1438501 |
| 3131. | Z80232 | Genbank | 1552545 |
| 3132. | Z82196 | Genbank | 6572205 |
| 3133. | Z82201 | Genbank | 1843447 |
| 3134. | Z82203 | Genbank | 3164070 |
| 3135. | Z82217 | Genbank | 2696013 |
| 3136. | Z83307 | Genbank | 1730464 |
| 3137. | Z83826 | Genbank | 4902653 |
| 3138. | Z83846 | Genbank | 3702439 |
| 3139. | Z84480 | Genbank | 3150089 |
| 3140. | Z84719 | Genbank | 2094785 |
| 3141. | Z84988 | Genbank | 1834699 |
| 3142. | Z85032 | Genbank | 1834743 |
| 3143. | Z86061 | Genbank | 2253035 |
| 3144. | Z92540 | Genbank | 2370068 |
| 3145. | Z92542 | Genbank | 4490841 |
| 3146. | Z93016 | Genbank | 9650703 |
| 3147. | Z93018 | Genbank | 2094788 |
| 3148. | Z93783 | Genbank | 4581336 |
| 3149. | Z93784 | Genbank | 2315175 |
| 3150. | Z93930 | Genbank | 4775603 |
| 3151. | Z94044 | Genbank | 2245342 |
| 3152. | Z94056 | Genbank | 2326510 |
| 3153. | Z95704 | Genbank | 2121307 |
| 3154. | Z97056 | Genbank | 2832593 |
| 3155. | Z98048 | Genbank | 2582746 |
| 3156. | Z98051 | Genbank | 5679749 |
| 3157. | Z98748 | Genbank | 2558551 |
| 3158. | Z98751 | Genbank | 2814366 |
| 3159. | Z98946 | Genbank | 4902640 |
| 3160. | Z99127 | Genbank | 2653426 |
| 3161. | Z99758 | Genbank | 4775628 |
| 3162. | Z99943 | Genbank | 2887308 |
| 3163. | AA001277 | EST | 1437430 |
| 3164. | AA001792 | EST | 1445606 |
| 3165. | AA005064 | EST | 1447761 |
| 3166. | AA005093 | EST | 1447790 |
| 3167. | AA010325 | EST | 1471512 |
| 3168. | AA010811 | EST | 1471867 |
| 3169. | AA011035 | EST | 1472082 |
| 3170. | AA011171 | EST | 1472198 |
| 3171. | AA018338 | EST | 1481805 |
| 3172. | AA018556 | EST | 1481956 |
| 3173. | AA021544 | EST | 1485428 |
| 3174. | AA024922 | EST | 1489828 |
| 3175. | AA026399 | EST | 1492300 |
| 3176. | AA026455 | EST | 1492355 |
| 3177. | AA029474 | EST | 1496896 |
| 3178. | AA035607 | EST | 1507554 |
| 3179. | AA037773 | EST | 1512881 |
| 3180. | AA040373 | EST | 1516650 |
| 3181. | AA043259 | EST | 1521133 |
| 3182. | AA047878 | EST | 1527574 |
| 3183. | AA047890 | EST | 1527586 |
| 3184. | AA053219 | EST | 1544560 |
| 3185. | AA056281 | EST | 1548685 |
| 3186. | AA056457 | EST | 1548797 |
| 3187. | AA065170 | EST | 1559065 |
| 3188. | AA071006 | EST | 1578559 |
| 3189. | AA074093 | EST | 1613980 |
| 3190. | AA079075 | EST | 1618159 |
| 3191. | AA082501 | EST | 1624682 |
| 3192. | AA083194 | EST | 1625455 |
| 3193. | AA084529 | EST | 1626585 |
| 3194. | AA085220 | EST | 1627340 |
| 3195. | AA089796 | EST | 1636288 |
| 3196. | AA101270 | EST | 1647951 |
| 3197. | AA112071 | EST | 1664157 |
| 3198. | AA121142 | EST | 1678881 |
| 3199. | AA128410 | EST | 1689708 |
| 3200. | AA129173 | EST | 1689008 |
| 3201. | AA131533 | EST | 1693084 |
| 3202. | AA131853 | EST | 1693343 |
| 3203. | AA135656 | EST | 1696695 |
| 3204. | AA136707 | EST | 1697917 |
| 3205. | AA136875 | EST | 1698085 |
| 3206. | AA147678 | EST | 1717118 |
| 3207. | AA148119 | EST | 1717494 |
| 3208. | AA148693 | EST | 1718872 |
| 3209. | AA149801 | EST | 1720899 |
| 3210. | AA150271 | EST | 1721792 |
| 3211. | AA151678 | EST | 1720233 |
| 3212. | AA152273 | EST | 1721676 |
| 3213. | AA164729 | EST | 1740889 |
| 3214. | AA166845 | EST | 1745080 |
| 3215. | AA167203 | EST | 1745770 |
| 3216. | AA169612 | EST | 1748000 |
| 3217. | AA178940 | EST | 1760293 |
| 3218. | AA179224 | EST | 1760576 |
| 3219. | AA181116 | EST | 1764599 |
| 3220. | AA187197 | EST | 1773608 |
| 3221. | AA187236 | EST | 1773462 |
| 3222. | AA188301 | EST | 1775387 |
| 3223. | AA193580 | EST | 1782981 |
| 3224. | AA207171 | EST | 1802663 |
| 3225. | AA213743 | EST | 1812361 |
| 3226. | AA214237 | EST | 1813058 |
| 3227. | AA226154 | EST | 1847470 |
| 3228. | AA227968 | EST | 1849595 |
| 3229. | AA228990 | EST | 1852023 |
| 3230. | AA233670 | EST | 1856663 |
| 3231. | AA235072 | EST | 1859508 |
| 3232. | AA258577 | EST | 1893701 |
| 3233. | AA259243 | EST | 1894695 |
| 3234. | AA280083 | EST | 1921557 |
| 3235. | AA281647 | EST | 1924345 |
| 3236. | AA284698 | EST | 1927389 |
| 3237. | AA287862 | EST | 1933570 |
| 3238. | AA291073 | EST | 1939180 |
| 3239. | AA292780 | EST | 1941602 |
| 3240. | AA313487 | EST | 1965816 |
| 3241. | AA313517 | EST | 1965918 |
| 3242. | AA316493 | EST | 1969063 |
| 3243. | AA320258 | EST | 1972618 |
| 3244. | AA328529 | EST | 1980794 |
| 3245. | AA342065 | EST | 1994300 |
| 3246. | AA343881 | EST | 1996117 |
| 3247. | AA393873 | EST | 2046842 |
| 3248. | AA397755 | EST | 2050583 |
| 3249. | AA399309 | EST | 2053046 |
| 3250. | AA406386 | EST | 2064369 |
| 3251. | AA412671 | EST | 2071277 |
| 3252. | AA418807 | EST | 2080608 |
| 3253. | AA420746 | EST | 2094625 |
| 3254. | AA427801 | EST | 2111617 |
| 3255. | AA428584 | EST | 2112581 |
| 3256. | AA429006 | EST | 2110641 |
| 3257. | AA429137 | EST | 2111912 |
| 3258. | AA431881 | EST | 2115589 |
| 3259. | AA434447 | EST | 2139361 |
| 3260. | AA435599 | EST | 2140513 |
| 3261. | AA442759 | EST | 2155434 |
| 3262. | AA443493 | EST | 2156168 |
| 3263. | AA447732 | EST | 2161402 |
| 3264. | AA453018 | EST | 2166687 |
| 3265. | AA453503 | EST | 2167172 |
| 3266. | AA455018 | EST | 2177794 |
| 3267. | AA455253 | EST | 2178029 |
| 3268. | AA455281 | EST | 2178057 |
| 3269. | AA455686 | EST | 2178462 |
| 3270. | AA457547 | EST | 2180267 |
| 3271. | AA458692 | EST | 2183599 |
| 3272. | AA468831 | EST | 2195365 |
| 3273. | AA469467 | EST | 2194262 |
| 3274. | AA469929 | EST | 2197238 |
| 3275. | AA470523 | EST | 2197832 |
| 3276. | AA476478 | EST | 2204689 |
| 3277. | AA482054 | EST | 2209732 |
| 3278. | AA490731 | EST | 2219904 |
| 3279. | AA491176 | EST | 2220349 |
| 3280. | AA492560 | EST | 2222122 |
| 3281. | AA494167 | EST | 2224008 |
| 3282. | AA495978 | EST | 2229299 |
| 3283. | AA508403 | EST | 2245906 |
| 3284. | AA512893 | EST | 2251316 |
| 3285. | AA516511 | EST | 2256035 |
| 3286. | AA523486 | EST | 2264198 |
| 3287. | AA524333 | EST | 2265261 |
| 3288. | AA527277 | EST | 2269346 |
| 3289. | AA531249 | EST | 2273955 |
| 3290. | AA534550 | EST | 2278803 |
| 3291. | AA535621 | EST | 2279874 |
| 3292. | AA541381 | EST | 2287815 |
| 3293. | AA541537 | EST | 2287971 |
| 3294. | AA551591 | EST | 2321843 |
| 3295. | AA554867 | EST | 2325406 |
| 3296. | AA570540 | EST | 2344520 |
| 3297. | AA574245 | EST | 2348760 |
| 3298. | AA576733 | EST | 2354207 |
| 3299. | AA587219 | EST | 2398033 |
| 3300. | AA595393 | EST | 2410743 |
| 3301. | AA600363 | EST | 2433988 |
| 3302. | AA603339 | EST | 2437200 |
| 3303. | AA608749 | EST | 2457177 |
| 3304. | AA608848 | EST | 2457276 |
| 3305. | AA620486 | EST | 2524425 |
| 3306. | AA621900 | EST | 2525776 |
| 3307. | AA622056 | EST | 2525932 |
| 3308. | AA622748 | EST | 2526624 |
| 3309. | AA625138 | EST | 2537523 |
| 3310. | AA625881 | EST | 2538268 |
| 3311. | AA629903 | EST | 2552514 |
| 3312. | AA630642 | EST | 2553253 |
| 3313. | AA630805 | EST | 2553416 |
| 3314. | AA631024 | EST | 2553635 |
| 3315. | AA635382 | EST | 2559224 |
| 3316. | AA641195 | EST | 2566445 |
| 3317. | AA644420 | EST | 2569638 |
| 3318. | AA652266 | EST | 2583918 |
| 3319. | AA657833 | EST | 2593987 |
| 3320. | AA668886 | EST | 2630385 |
| 3321. | AA677091 | EST | 2657613 |
| 3322. | AA680147 | EST | 2656614 |
| 3323. | AA687226 | EST | 2675417 |
| 3324. | AA689292 | EST | 2690536 |
| 3325. | AA693886 | EST | 2694824 |
| 3326. | AA694160 | EST | 2695098 |
| 3327. | AA707101 | EST | 2717019 |
| 3328. | AA723833 | EST | 2741540 |
| 3329. | AA729028 | EST | 2750387 |
| 3330. | AA738104 | EST | 2768861 |
| 3331. | AA741565 | EST | 2780157 |
| 3332. | AA745246 | EST | 2785232 |
| 3333. | AA745714 | EST | 2785700 |
| 3334. | AA747577 | EST | 2787535 |
| 3335. | AA748063 | EST | 2788021 |
| 3336. | AA748805 | EST | 2788763 |
| 3337. | AA758966 | EST | 2806829 |
| 3338. | AA760912 | EST | 2809842 |
| 3339. | AA775446 | EST | 2834780 |
| 3340. | AA806720 | EST | 2875470 |
| 3341. | AA811666 | EST | 2881277 |
| 3342. | AA813221 | EST | 2883206 |
| 3343. | AA813244 | EST | 2883229 |
| 3344. | AA824613 | EST | 2896635 |
| 3345. | AA828278 | EST | 2901377 |
| 3346. | AA831497 | EST | 2904596 |
| 3347. | AA832512 | EST | 2898463 |
| 3348. | AA833760 | EST | 2908528 |
| 3349. | AA834584 | EST | 2908183 |
| 3350. | AA835660 | EST | 2909979 |
| 3351. | AA836765 | EST | 2911964 |
| 3352. | AA845366 | EST | 2933125 |
| 3353. | AA846613 | EST | 2932753 |
| 3354. | AA857333 | EST | 2945635 |
| 3355. | AA879430 | EST | 2988541 |
| 3356. | AA884309 | EST | 2993839 |
| 3357. | AA887132 | EST | 3002240 |
| 3358. | AA888589 | EST | 3004264 |
| 3359. | AA903067 | EST | 3038190 |
| 3360. | AA903389 | EST | 3038512 |
| 3361. | AA903767 | EST | 3038890 |
| 3362. | AA903894 | EST | 3039017 |
| 3363. | AA910949 | EST | 3050239 |
| 3364. | AA911115 | EST | 3050405 |
| 3365. | AA916271 | EST | 3055663 |
| 3366. | AA916393 | EST | 3055785 |
| 3367. | AA947758 | EST | 3109011 |
| 3368. | AA961215 | EST | 3127769 |
| 3369. | AA975192 | EST | 3150984 |
| 3370. | AA976004 | EST | 3151796 |
| 3371. | AA993434 | EST | 3179979 |
| 3372. | AA995358 | EST | 3181847 |
| 3373. | AF001540 | EST | 2529712 |
| 3374. | AF062713 | EST | 4731769 |
| 3375. | AF150234 | EST | 5133670 |
| 3376. | AI003516 | EST | 3203055 |
| 3377. | AI004311 | EST | 3213821 |
| 3378. | AI017011 | EST | 3231347 |
| 3379. | AI018024 | EST | 3232360 |
| 3380. | AI023741 | EST | 3238785 |
| 3381. | AI023786 | EST | 3238830 |
| 3382. | AI024757 | EST | 3240370 |
| 3383. | AI053751 | EST | 3321538 |
| 3384. | AI061223 | EST | 3336591 |
| 3385. | AI074253 | EST | 3400897 |
| 3386. | AI081636 | EST | 3418428 |
| 3387. | AI089782 | EST | 3428841 |
| 3388. | AI092862 | EST | 3431838 |
| 3389. | AI095447 | EST | 3434423 |
| 3390. | AI130847 | EST | 3600863 |
| 3391. | AI132935 | EST | 6360251 |
| 3392. | AI133695 | EST | 6361011 |
| 3393. | AI138207 | EST | 3644179 |
| 3394. | AI140618 | EST | 3648075 |
| 3395. | AI143587 | EST | 3665396 |
| 3396. | AI148051 | EST | 3675733 |
| 3397. | AI151016 | EST | 3679485 |
| 3398. | AI174394 | EST | 3721247 |
| 3399. | AI174693 | EST | 6361071 |
| 3400. | AI186319 | EST | 3736957 |
| 3401. | AI199982 | EST | 3752588 |
| 3402. | AI207956 | EST | 3769898 |
| 3403. | AI224992 | EST | 3807705 |
| 3404. | AI239975 | EST | 3835372 |
| 3405. | AI241763 | EST | 3837160 |
| 3406. | AI244380 | EST | 3839777 |
| 3407. | AI246319 | EST | 3841716 |
| 3408. | AI247193 | EST | 3842590 |
| 3409. | AI249323 | EST | 3845852 |
| 3410. | AI249800 | EST | 3846329 |
| 3411. | AI251434 | EST | 3847963 |
| 3412. | AI254731 | EST | 3862256 |
| 3413. | AI261857 | EST | 3870060 |
| 3414. | AI264741 | EST | 3872944 |
| 3415. | AI267341 | EST | 3886508 |
| 3416. | AI269205 | EST | 3888372 |
| 3417. | AI269580 | EST | 3888747 |
| 3418. | AI269862 | EST | 3889029 |
| 3419. | AI270657 | EST | 3889824 |
| 3420. | AI271786 | EST | 3890953 |
| 3421. | AI273048 | EST | 3895316 |
| 3422. | AI273964 | EST | 3896232 |
| 3423. | AI274508 | EST | 3896776 |
| 3424. | AI274728 | EST | 3897002 |
| 3425. | AI275175 | EST | 3897449 |
| 3426. | AI275686 | EST | 3897960 |
| 3427. | AI276121 | EST | 3898395 |
| 3428. | AI276172 | EST | 3898446 |
| 3429. | AI279906 | EST | 3918140 |
| 3430. | AI280747 | EST | 3918980 |
| 3431. | AI281762 | EST | 3919995 |
| 3432. | AI281837 | EST | 3920070 |
| 3433. | AI282281 | EST | 3920514 |
| 3434. | AI282326 | EST | 3920559 |
| 3435. | AI282688 | EST | 3920921 |
| 3436. | AI282695 | EST | 3920928 |
| 3437. | AI290325 | EST | 3933099 |
| 3438. | AI312325 | EST | 4017930 |
| 3439. | AI334738 | EST | 4071665 |
| 3440. | AI334902 | EST | 4071829 |
| 3441. | AI339038 | EST | 4075965 |
| 3442. | AI341838 | EST | 4078765 |
| 3443. | AI342746 | EST | 4079952 |
| 3444. | AI343037 | EST | 4080243 |
| 3445. | AI343112 | EST | 4080318 |
| 3446. | AI351382 | EST | 4088588 |
| 3447. | AI357115 | EST | 4108736 |
| 3448. | AI374447 | EST | 4164959 |
| 3449. | AI375640 | EST | 4175630 |
| 3450. | AI375702 | EST | 4175692 |
| 3451. | AI379869 | EST | 4189722 |
| 3452. | AI420521 | EST | 4266452 |
| 3453. | AI421654 | EST | 4267585 |
| 3454. | AI432218 | EST | 4308644 |
| 3455. | AI432304 | EST | 4309247 |
| 3456. | AI432656 | EST | 4283443 |
| 3457. | AI433023 | EST | 4286271 |
| 3458. | AI433261 | EST | 4287937 |
| 3459. | AI434020 | EST | 4293255 |
| 3460. | AI435500 | EST | 4303618 |
| 3461. | AI439717 | EST | 4306274 |
| 3462. | AI440263 | EST | 4281427 |
| 3463. | AI445131 | EST | 4286848 |
| 3464. | AI445829 | EST | 4290946 |
| 3465. | AI446597 | EST | 4296324 |
| 3466. | AI457135 | EST | 4310004 |
| 3467. | AI458675 | EST | 4311254 |
| 3468. | AI459581 | EST | 4312462 |
| 3469. | AI469468 | EST | 4331558 |
| 3470. | AI471527 | EST | 4333617 |
| 3471. | AI472120 | EST | 4334210 |
| 3472. | AI475134 | EST | 4328179 |
| 3473. | AI476305 | EST | 4329350 |
| 3474. | AI480414 | EST | 4373582 |
| 3475. | AI492488 | EST | 4393491 |
| 3476. | AI499986 | EST | 4391968 |
| 3477. | AI500077 | EST | 4392059 |
| 3478. | AI500659 | EST | 4392641 |
| 3479. | AI521275 | EST | 4435410 |
| 3480. | AI538790 | EST | 4452925 |
| 3481. | AI539071 | EST | 4453206 |
| 3482. | AI567935 | EST | 4526387 |
| 3483. | AI568855 | EST | 4532229 |
| 3484. | AI570786 | EST | 4534160 |
| 3485. | AI571861 | EST | 4535235 |
| 3486. | AI580927 | EST | 4565303 |
| 3487. | AI587289 | EST | 4573730 |
| 3488. | AI589391 | EST | 4598439 |
| 3489. | AI590557 | EST | 4599605 |
| 3490. | AI591201 | EST | 4600249 |
| 3491. | AI597750 | EST | 4606798 |
| 3492. | AI609375 | EST | 4618542 |
| 3493. | AI610362 | EST | 4619529 |
| 3494. | AI612721 | EST | 4621888 |
| 3495. | AI613492 | EST | 4622659 |
| 3496. | AI620003 | EST | 4629129 |
| 3497. | AI620270 | EST | 4629396 |
| 3498. | AI624950 | EST | 4649881 |
| 3499. | AI625316 | EST | 4650247 |
| 3500. | AI628815 | EST | 4665615 |
| 3501. | AI630747 | EST | 4682077 |
| 3502. | AI630827 | EST | 4682157 |
| 3503. | AI633388 | EST | 4684718 |
| 3504. | AI634293 | EST | 4685623 |
| 3505. | AI637919 | EST | 4690153 |
| 3506. | AI638096 | EST | 4690330 |
| 3507. | AI648473 | EST | 4729307 |
| 3508. | AI650787 | EST | 4734766 |
| 3509. | AI657001 | EST | 4740980 |
| 3510. | AI670846 | EST | 4850577 |
| 3511. | AI677810 | EST | 4887992 |
| 3512. | AI680226 | EST | 4890408 |
| 3513. | AI680458 | EST | 4890640 |
| 3514. | AI681558 | EST | 4891740 |
| 3515. | AI682779 | EST | 4892961 |
| 3516. | AI683340 | EST | 4893522 |
| 3517. | AI689640 | EST | 4900934 |
| 3518. | AI690043 | EST | 4901337 |
| 3519. | AI695292 | EST | 4983192 |
| 3520. | AI701480 | EST | 4989380 |
| 3521. | AI702433 | EST | 4990333 |
| 3522. | AI735341 | EST | 5056865 |
| 3523. | AI741480 | EST | 5109768 |
| 3524. | AI744509 | EST | 5112797 |
| 3525. | AI745605 | EST | 5113893 |
| 3526. | AI749022 | EST | 5127221 |
| 3527. | AI754605 | EST | 5132869 |
| 3528. | AI758437 | EST | 5152160 |
| 3529. | AI760357 | EST | 5176024 |
| 3530. | AI762216 | EST | 5177883 |
| 3531. | AI762247 | EST | 5177914 |
| 3532. | AI765871 | EST | 5232380 |
| 3533. | AI783504 | EST | 5325313 |
| 3534. | AI784252 | EST | 5326061 |
| 3535. | AI799305 | EST | 5364777 |
| 3536. | AI801152 | EST | 5366624 |
| 3537. | AI802372 | EST | 5367844 |
| 3538. | AI803205 | EST | 5368677 |
| 3539. | AI819309 | EST | 5438388 |
| 3540. | AI823585 | EST | 5444256 |
| 3541. | AI824566 | EST | 5445237 |
| 3542. | AI827311 | EST | 5447982 |
| 3543. | AI830029 | EST | 5450700 |
| 3544. | AI869367 | EST | 5543335 |
| 3545. | AI869639 | EST | 5543607 |
| 3546. | AI870523 | EST | 5544491 |
| 3547. | AI871849 | EST | 5545898 |
| 3548. | AI871981 | EST | 5546030 |
| 3549. | AI878836 | EST | 5552885 |
| 3550. | AI885879 | EST | 5591043 |
| 3551. | AI887430 | EST | 5592594 |
| 3552. | AI887664 | EST | 5592828 |
| 3553. | AI887776 | EST | 5592940 |
| 3554. | AI905464 | EST | 6495851 |
| 3555. | AI912666 | EST | 5632521 |
| 3556. | AI925156 | EST | 5661120 |
| 3557. | AI948813 | EST | 5741123 |
| 3558. | AI982630 | EST | 5809849 |
| 3559. | AI984360 | EST | 5811637 |
| 3560. | AI990911 | EST | 5837806 |
| 3561. | AL039945 | EST | 5408928 |
| 3562. | AL043913 | EST | 5432141 |
| 3563. | AL046836 | EST | 5936250 |
| 3564. | AL048871 | EST | 5935406 |
| 3565. | AL118639 | EST | 5924538 |
| 3566. | AL121193 | EST | 5927194 |
| 3567. | AL157508 | EST | 7057909 |
| 3568. | AV646708 | EST | 9867722 |
| 3569. | AV648443 | EST | 9869457 |
| 3570. | AV652443 | EST | 9873457 |
| 3571. | AV691783 | EST | 10293646 |
| 3572. | AW005030 | EST | 5853808 |
| 3573. | AW008472 | EST | 5857250 |
| 3574. | AW015018 | EST | 5863775 |
| 3575. | AW022615 | EST | 5876145 |
| 3576. | AW028516 | EST | 5887272 |
| 3577. | AW029349 | EST | 5888105 |
| 3578. | AW069569 | EST | 6024567 |
| 3579. | AW080079 | EST | 6035231 |
| 3580. | AW089572 | EST | 6046916 |
| 3581. | AW089689 | EST | 6047033 |
| 3582. | AW117743 | EST | 6086327 |
| 3583. | AW138881 | EST | 6143199 |
| 3584. | AW157183 | EST | 6228584 |
| 3585. | AW168795 | EST | 6400320 |
| 3586. | AW169658 | EST | 6401183 |
| 3587. | AW172722 | EST | 6438670 |
| 3588. | AW173161 | EST | 6439109 |
| 3589. | AW188031 | EST | 6462467 |
| 3590. | AW192906 | EST | 6471605 |
| 3591. | AW195881 | EST | 6475111 |
| 3592. | AW204876 | EST | 6504348 |
| 3593. | AW243297 | EST | 6577137 |
| 3594. | AW249638 | EST | 6592631 |
| 3595. | AW302552 | EST | 6712232 |
| 3596. | AW362091 | EST | 6866741 |
| 3597. | AW369732 | EST | 6874386 |
| 3598. | AW406841 | EST | 6925898 |
| 3599. | AW513892 | EST | 7151970 |
| 3600. | AW516241 | EST | 7154323 |
| 3601. | AW572048 | EST | 7236779 |
| 3602. | AW574774 | EST | 7246313 |
| 3603. | AW615336 | EST | 7320522 |
| 3604. | AW748399 | EST | 7663331 |
| 3605. | AW752836 | EST | 7667768 |
| 3606. | AW753919 | EST | 7668851 |
| 3607. | AW771845 | EST | 7703905 |
| 3608. | AW793729 | EST | 7845599 |
| 3609. | AW802240 | EST | 7854110 |
| 3610. | AW809560 | EST | 7902554 |
| 3611. | AW814692 | EST | 7907686 |
| 3612. | AW852432 | EST | 7947949 |
| 3613. | AW853953 | EST | 7949646 |
| 3614. | AW889913 | EST | 8054118 |
| 3615. | AW901289 | EST | 8065598 |
| 3616. | AW949539 | EST | 8139169 |
| 3617. | AW954169 | EST | 8143852 |
| 3618. | AW955462 | EST | 8145145 |
| 3619. | AW956540 | EST | 8146223 |
| 3620. | AW958798 | EST | 8148482 |
| 3621. | AW962143 | EST | 8151901 |
| 3622. | AW962511 | EST | 8152347 |
| 3623. | AW963309 | EST | 8153145 |
| 3624. | AW966042 | EST | 8155878 |
| 3625. | AW975215 | EST | 8166423 |
| 3626. | AW998156 | EST | 8258390 |
| 3627. | BE003583 | EST | 8263816 |
| 3628. | BE073702 | EST | 8420932 |
| 3629. | BE080122 | EST | 8470409 |
| 3630. | BE145038 | EST | 8607762 |
| 3631. | BE161669 | EST | 8624390 |
| 3632. | BE175873 | EST | 8638602 |
| 3633. | BE274196 | EST | 9149136 |
| 3634. | BE380117 | EST | 9325482 |
| 3635. | BE393497 | EST | 9338862 |
| 3636. | BE538686 | EST | 9767331 |
| 3637. | BE539191 | EST | 9767836 |
| 3638. | BE543906 | EST | 9772551 |
| 3639. | BE550805 | EST | 9792497 |
| 3640. | BE564779 | EST | 9808499 |
| 3641. | BE565183 | EST | 9808903 |
| 3642. | BE567133 | EST | 9810853 |
| 3643. | BE568411 | EST | 9812131 |
| 3644. | BE670287 | EST | 10030828 |
| 3645. | BE695938 | EST | 10083111 |
| 3646. | BE708268 | EST | 10096533 |
| 3647. | BE709664 | EST | 10097929 |
| 3648. | BE717766 | EST | 10106044 |
| 3649. | BE739633 | EST | 10153625 |
| 3650. | BE837515 | EST | 10269893 |
| 3651. | D54603 | EST | 956500 |
| 3652. | D59851 | EST | 960957 |
| 3653. | H72652 | EST | 1044468 |
| 3654. | H92571 | EST | 1088149 |
| 3655. | N25067 | EST | 1139217 |
| 3656. | N36289 | EST | 1157431 |
| 3657. | N41827 | EST | 1165858 |
| 3658. | N58888 | EST | 1202778 |
| 3659. | N74353 | EST | 1231638 |
| 3660. | R25849 | EST | 781984 |
| 3661. | R32646 | EST | 788489 |
| 3662. | R49117 | EST | 820187 |
| 3663. | R89779 | EST | 954606 |
| 3664. | T96395 | EST | 735019 |
| 3665. | W94483 | EST | 1423762 |
| 3666. | A00475 | NUCPATENT | 1566707 |
| 3667. | A06349 | NUCPATENT | 412832 |
| 3668. | A23431 | NUCPATENT | 1247591 |
| 3669. | A26323 | NUCPATENT | 904887 |
| 3670. | N91467 | NUCPATENT | 1444794 |
| 3671. | T39279 | NUCPATENT | 647033 |
| 3672. | V05384 | NUCPATENT | N/A |
| 3673. | V59659 | NUCPATENT | N/A |
| 3674. | V84596 | NUCPATENT | N/A |
| 3675. | V89126 | NUCPATENT | N/A |
| 3676. | X04323 | NUCPATENT | 16478 |
| 3677. | X99865 | NUCPATENT | 1881527 |
| 3678. | Z52972 | NUCPATENT | 1234272 |
| 3679. | Z58977 | NUCPATENT | 1030890 |
| 3680. | Z65287 | NUCPATENT | 1038109 |
| 3681. | Z86967 | NUCPATENT | 1883879 |
| 3682. | Z94893 | NUCPATENT | 2463302 |
| 3683. | AC46107 | PREPATNUC | N/A |
| 3684. | A02759 | Genbank | 345130 |
| 3685. | A36461 | Genbank | 2293779 |
| 3686. | A69527 | Genbank | 4774176 |
| 3687. | A94982 | Genbank | 6779163 |
| 3688. | AB000516 | Genbank | 2723379 |
| 3689. | AB000888 | Genbank | 2467297 |
| 3690. | AB001901 | Genbank | 2281756 |
| 3691. | AB002282 | Genbank | 6526354 |
| 3692. | AB002303 | Genbank | 2224550 |
| 3693. | AB002323 | Genbank | 2224590 |
| 3694. | AB002351 | Genbank | 2224646 |
| 3695. | AB002365 | Genbank | 2224674 |
| 3696. | AB002368 | Genbank | 2224680 |
| 3697. | AB002391 | Genbank | 6683696 |
| 3698. | AB003151 | Genbank | 3702678 |
| 3699. | AB004066 | Genbank | 2308996 |
| 3700. | AB004317 | Genbank | 3036927 |
| 3701. | AB005047 | Genbank | 3116213 |
| 3702. | AB007884 | Genbank | 2887422 |
| 3703. | AB007885 | Genbank | 2887424 |
| 3704. | AB007889 | Genbank | 2887432 |
| 3705. | AB007899 | Genbank | 2662158 |
| 3706. | AB007901 | Genbank | 2662162 |
| 3707. | AB007944 | Genbank | 3413911 |
| 3708. | AB009282 | Genbank | 2662290 |
| 3709. | AB011085 | Genbank | 3043549 |
| 3710. | AB011100 | Genbank | 6683714 |
| 3711. | AB011105 | Genbank | 3043589 |
| 3712. | AB011135 | Genbank | 3043649 |
| 3713. | AB011161 | Genbank | 3043701 |
| 3714. | AB011399 | Genbank | 3452571 |
| 3715. | AB011421 | Genbank | 3834355 |
| 3716. | AB014525 | Genbank | 3327063 |
| 3717. | AB014560 | Genbank | 3327133 |
| 3718. | AB014583 | Genbank | 6635130 |
| 3719. | AB014603 | Genbank | 3327219 |
| 3720. | AB015856 | Genbank | 3953530 |
| 3721. | AB017019 | Genbank | 4512256 |
| 3722. | AB017602 | Genbank | 6681700 |
| 3723. | AB018266 | Genbank | 3882166 |
| 3724. | AB018283 | Genbank | 6705974 |
| 3725. | AB018295 | Genbank | 3882224 |
| 3726. | AB018309 | Genbank | 3882252 |
| 3727. | AB018331 | Genbank | 3882296 |
| 3728. | AB018341 | Genbank | 3882316 |
| 3729. | AB020662 | Genbank | 4240198 |
| 3730. | AB020687 | Genbank | 4240248 |
| 3731. | AB020693 | Genbank | 4240260 |
| 3732. | AB020723 | Genbank | 4240320 |
| 3733. | AB020777 | Genbank | 6467211 |
| 3734. | AB020867 | Genbank | 4003387 |
| 3735. | AB020880 | Genbank | 4996281 |
| 3736. | AB021288 | Genbank | 4038732 |
| 3737. | AB021868 | Genbank | 4996562 |
| 3738. | AB022435 | Genbank | 4996607 |
| 3739. | AB023049 | Genbank | 5672604 |
| 3740. | AB023163 | Genbank | 4589535 |
| 3741. | AB023197 | Genbank | 4589603 |
| 3742. | AB023208 | Genbank | 4589625 |
| 3743. | AB023222 | Genbank | 4589653 |
| 3744. | AB023420 | Genbank | 4579908 |
| 3745. | AB024334 | Genbank | 6016837 |
| 3746. | AB024597 | Genbank | 7209838 |
| 3747. | AB026898 | Genbank | 4835382 |
| 3748. | AB027466 | Genbank | 6172220 |
| 3749. | AB028128 | Genbank | 8100053 |
| 3750. | AB028893 | Genbank | 6552364 |
| 3751. | AB028986 | Genbank | 5689462 |
| 3752. | AB029020 | Genbank | 5689530 |
| 3753. | AB029023 | Genbank | 5689536 |
| 3754. | AB029027 | Genbank | 5689544 |
| 3755. | AB029636 | Genbank | 9836647 |
| 3756. | AB030653 | Genbank | 5689376 |
| 3757. | AB030814 | Genbank | 9757509 |
| 3758. | AB030815 | Genbank | 9757511 |
| 3759. | AB032971 | Genbank | 6330018 |
| 3760. | AB032998 | Genbank | 6330210 |
| 3761. | AB033042 | Genbank | 6330568 |
| 3762. | AB033050 | Genbank | 6330623 |
| 3763. | AB033070 | Genbank | 6330818 |
| 3764. | AB033087 | Genbank | 6330947 |
| 3765. | AB037729 | Genbank | 7242970 |
| 3766. | AB037742 | Genbank | 7243022 |
| 3767. | AB037772 | Genbank | 7243082 |
| 3768. | AB037797 | Genbank | 7243132 |
| 3769. | AB037807 | Genbank | 7243152 |
| 3770. | AB037857 | Genbank | 7243269 |
| 3771. | AB040927 | Genbank | 7959248 |
| 3772. | AB046801 | Genbank | 10047236 |
| 3773. | AB046821 | Genbank | 10047276 |
| 3774. | AB046830 | Genbank | 10047294 |
| 3775. | AB046861 | Genbank | 10047358 |
| 3776. | AC000021 | Genbank | 2133892 |
| 3777. | AC000062 | Genbank | 1669374 |
| 3778. | AC000066 | Genbank | 3645943 |
| 3779. | AC000353 | Genbank | 6970735 |
| 3780. | AC000378 | Genbank | 2270906 |
| 3781. | AC000386 | Genbank | 2431612 |
| 3782. | AC001228 | Genbank | 1935053 |
| 3783. | AC001234 | Genbank | 4028939 |
| 3784. | AC002039 | Genbank | 2342716 |
| 3785. | AC002044 | Genbank | 2347079 |
| 3786. | AC002094 | Genbank | 2155224 |
| 3787. | AC002119 | Genbank | 2599244 |
| 3788. | AC002314 | Genbank | 2252637 |
| 3789. | AC002326 | Genbank | 2288970 |
| 3790. | AC002349 | Genbank | 2809270 |
| 3791. | AC002366 | Genbank | 2739349 |
| 3792. | AC002381 | Genbank | 2275186 |
| 3793. | AC002383 | Genbank | 3947432 |
| 3794. | AC002395 | Genbank | 3540146 |
| 3795. | AC002403 | Genbank | 2598081 |
| 3796. | AC002404 | Genbank | 2329922 |
| 3797. | AC002418 | Genbank | 2822137 |
| 3798. | AC002428 | Genbank | 2335068 |
| 3799. | AC002429 | Genbank | 2335067 |
| 3800. | AC002430 | Genbank | 2335066 |
| 3801. | AC002449 | Genbank | 2337886 |
| 3802. | AC002450 | Genbank | 2337884 |
| 3803. | AC002452 | Genbank | 2337880 |
| 3804. | AC002460 | Genbank | 2337869 |
| 3805. | AC002461 | Genbank | 2337868 |
| 3806. | AC002480 | Genbank | 2340098 |
| 3807. | AC002485 | Genbank | 2341016 |
| 3808. | AC002528 | Genbank | 2388554 |
| 3809. | AC002558 | Genbank | 2580474 |
| 3810. | AC002978 | Genbank | 2961441 |
| 3811. | AC003003 | Genbank | 2865207 |
| 3812. | AC003006 | Genbank | 2529399 |
| 3813. | AC003007 | Genbank | 2911728 |
| 3814. | AC003029 | Genbank | 9965520 |
| 3815. | AC003035 | Genbank | 4204245 |
| 3816. | AC003037 | Genbank | 2920805 |
| 3817. | AC003041 | Genbank | 3264572 |
| 3818. | AC003077 | Genbank | 2588634 |
| 3819. | AC003080 | Genbank | 2588627 |
| 3820. | AC003085 | Genbank | 2588620 |
| 3821. | AC003098 | Genbank | 2822155 |
| 3822. | AC003100 | Genbank | 2618658 |
| 3823. | AC003108 | Genbank | 2833632 |
| 3824. | AC003663 | Genbank | 3097871 |
| 3825. | AC003666 | Genbank | 2992476 |
| 3826. | AC003682 | Genbank | 3264845 |
| 3827. | AC003953 | Genbank | 2708755 |
| 3828. | AC003959 | Genbank | 2731598 |
| 3829. | AC003989 | Genbank | 2772538 |
| 3830. | AC003991 | Genbank | 2772535 |
| 3831. | AC003999 | Genbank | 2772566 |
| 3832. | AC004055 | Genbank | 3482937 |
| 3833. | AC004061 | Genbank | 3108028 |
| 3834. | AC004074 | Genbank | 3046270 |
| 3835. | AC004080 | Genbank | 2822164 |
| 3836. | AC004084 | Genbank | 2822156 |
| 3837. | AC004098 | Genbank | 3097872 |
| 3838. | AC004099 | Genbank | 3650064 |
| 3839. | AC004103 | Genbank | 3063487 |
| 3840. | AC004130 | Genbank | 2842785 |
| 3841. | AC004152 | Genbank | 2894631 |
| 3842. | AC004166 | Genbank | 8887011 |
| 3843. | AC004227 | Genbank | 2911718 |
| 3844. | AC004230 | Genbank | 4028938 |
| 3845. | AC004381 | Genbank | 2982169 |
| 3846. | AC004382 | Genbank | 3252819 |
| 3847. | AC004383 | Genbank | 2944111 |
| 3848. | AC004408 | Genbank | 3873185 |
| 3849. | AC004451 | Genbank | 2979604 |
| 3850. | AC004453 | Genbank | 2979600 |
| 3851. | AC004457 | Genbank | 2979596 |
| 3852. | AC004472 | Genbank | 2984582 |
| 3853. | AC004474 | Genbank | 3097873 |
| 3854. | AC004477 | Genbank | 3688107 |
| 3855. | AC004485 | Genbank | 2992497 |
| 3856. | AC004526 | Genbank | 3779035 |
| 3857. | AC004528 | Genbank | 3025444 |
| 3858. | AC004537 | Genbank | 3041854 |
| 3859. | AC004538 | Genbank | 3041853 |
| 3860. | AC004540 | Genbank | 3041850 |
| 3861. | AC004542 | Genbank | 3041846 |
| 3862. | AC004544 | Genbank | 3041843 |
| 3863. | AC004549 | Genbank | 3046306 |
| 3864. | AC004582 | Genbank | 3337307 |
| 3865. | AC004583 | Genbank | 3293210 |
| 3866. | AC004614 | Genbank | 3080662 |
| 3867. | AC004651 | Genbank | 3097833 |
| 3868. | AC004686 | Genbank | 3688105 |
| 3869. | AC004687 | Genbank | 3258613 |
| 3870. | AC004741 | Genbank | 3152631 |
| 3871. | AC004742 | Genbank | 3152630 |
| 3872. | AC004765 | Genbank | 4731046 |
| 3873. | AC004768 | Genbank | 3169153 |
| 3874. | AC004771 | Genbank | 3668108 |
| 3875. | AC004772 | Genbank | 11128432 |
| 3876. | AC004779 | Genbank | 3493170 |
| 3877. | AC004782 | Genbank | 3172145 |
| 3878. | AC004804 | Genbank | 3406032 |
| 3879. | AC004820 | Genbank | 6175373 |
| 3880. | AC004821 | Genbank | 11181836 |
| 3881. | AC004825 | Genbank | 4753289 |
| 3882. | AC004846 | Genbank | 7243869 |
| 3883. | AC004848 | Genbank | 3900858 |
| 3884. | AC004849 | Genbank | 3980553 |
| 3885. | AC004865 | Genbank | 4156195 |
| 3886. | AC004876 | Genbank | 4508148 |
| 3887. | AC004881 | Genbank | 3900853 |
| 3888. | AC004884 | Genbank | 4156189 |
| 3889. | AC004895 | Genbank | 4926908 |
| 3890. | AC004896 | Genbank | 3845414 |
| 3891. | AC004902 | Genbank | 5757546 |
| 3892. | AC004904 | Genbank | 4156180 |
| 3893. | AC004918 | Genbank | 4153871 |
| 3894. | AC004919 | Genbank | 3638950 |
| 3895. | AC004921 | Genbank | 4156176 |
| 3896. | AC004923 | Genbank | 4263742 |
| 3897. | AC004924 | Genbank | 4263741 |
| 3898. | AC004926 | Genbank | 4263559 |
| 3899. | AC004943 | Genbank | 3924671 |
| 3900. | AC004954 | Genbank | 3818410 |
| 3901. | AC004955 | Genbank | 4753267 |
| 3902. | AC004968 | Genbank | 4156160 |
| 3903. | AC004969 | Genbank | 4156159 |
| 3904. | AC004974 | Genbank | 3970965 |
| 3905. | AC004982 | Genbank | 3419846 |
| 3906. | AC004985 | Genbank | 5708490 |
| 3907. | AC004989 | Genbank | 3900851 |
| 3908. | AC004994 | Genbank | 3900849 |
| 3909. | AC005007 | Genbank | 4156151 |
| 3910. | AC005034 | Genbank | 3947437 |
| 3911. | AC005041 | Genbank | 4508118 |
| 3912. | AC005042 | Genbank | 4156138 |
| 3913. | AC005043 | Genbank | 4926890 |
| 3914. | AC005046 | Genbank | 6094632 |
| 3915. | AC005060 | Genbank | 10445386 |
| 3916. | AC005067 | Genbank | 4508114 |
| 3917. | AC005070 | Genbank | 3406049 |
| 3918. | AC005071 | Genbank | 4508112 |
| 3919. | AC005074 | Genbank | 4153859 |
| 3920. | AC005075 | Genbank | 4926889 |
| 3921. | AC005083 | Genbank | 4150930 |
| 3922. | AC005088 | Genbank | 4753221 |
| 3923. | AC005099 | Genbank | 4150927 |
| 3924. | AC005104 | Genbank | 4218027 |
| 3925. | AC005158 | Genbank | 5091650 |
| 3926. | AC005164 | Genbank | 3242749 |
| 3927. | AC005176 | Genbank | 3253115 |
| 3928. | AC005182 | Genbank | 4558525 |
| 3929. | AC005192 | Genbank | 3264575 |
| 3930. | AC005203 | Genbank | 3273381 |
| 3931. | AC005210 | Genbank | 6249673 |
| 3932. | AC005212 | Genbank | 3287444 |
| 3933. | AC005230 | Genbank | 4156158 |
| 3934. | AC005231 | Genbank | 4731071 |
| 3935. | AC005239 | Genbank | 3287673 |
| 3936. | AC005247 | Genbank | 3287723 |
| 3937. | AC005249 | Genbank | 3287720 |
| 3938. | AC005283 | Genbank | 7243875 |
| 3939. | AC005301 | Genbank | 7107554 |
| 3940. | AC005306 | Genbank | 3334980 |
| 3941. | AC005316 | Genbank | 3738342 |
| 3942. | AC005324 | Genbank | 3366582 |
| 3943. | AC005325 | Genbank | 3366581 |
| 3944. | AC005368 | Genbank | 3367506 |
| 3945. | AC005409 | Genbank | 4249432 |
| 3946. | AC005488 | Genbank | 5836194 |
| 3947. | AC005510 | Genbank | 4432790 |
| 3948. | AC005517 | Genbank | 7670171 |
| 3949. | AC005525 | Genbank | 3451333 |
| 3950. | AC005531 | Genbank | 4153875 |
| 3951. | AC005534 | Genbank | 4753272 |
| 3952. | AC005538 | Genbank | 4508129 |
| 3953. | AC005544 | Genbank | 3650045 |
| 3954. | AC005550 | Genbank | 3478668 |
| 3955. | AC005551 | Genbank | 3482904 |
| 3956. | AC005562 | Genbank | 4153858 |
| 3957. | AC005567 | Genbank | 3493327 |
| 3958. | AC005570 | Genbank | 3510231 |
| 3959. | AC005587 | Genbank | 4156166 |
| 3960. | AC005592 | Genbank | 3513292 |
| 3961. | AC005595 | Genbank | 3513299 |
| 3962. | AC005596 | Genbank | 3513298 |
| 3963. | AC005609 | Genbank | 3540156 |
| 3964. | AC005618 | Genbank | 3548785 |
| 3965. | AC005629 | Genbank | 7243877 |
| 3966. | AC005632 | Genbank | 4827304 |
| 3967. | AC005663 | Genbank | 7126292 |
| 3968. | AC005667 | Genbank | 3641773 |
| 3969. | AC005670 | Genbank | 3688099 |
| 3970. | AC005690 | Genbank | 6850335 |
| 3971. | AC005696 | Genbank | 3819097 |
| 3972. | AC005702 | Genbank | 3688100 |
| 3973. | AC005704 | Genbank | 3659406 |
| 3974. | AC005722 | Genbank | 3738108 |
| 3975. | AC005726 | Genbank | 3810672 |
| 3976. | AC005752 | Genbank | 3688076 |
| 3977. | AC005755 | Genbank | 3688073 |
| 3978. | AC005785 | Genbank | 3702290 |
| 3979. | AC005829 | Genbank | 3873300 |
| 3980. | AC005831 | Genbank | 4165331 |
| 3981. | AC005837 | Genbank | 3849820 |
| 3982. | AC005844 | Genbank | 4726091 |
| 3983. | AC005871 | Genbank | 6249681 |
| 3984. | AC005876 | Genbank | 6249672 |
| 3985. | AC005877 | Genbank | 6249671 |
| 3986. | AC005905 | Genbank | 4090182 |
| 3987. | AC005913 | Genbank | 9966229 |
| 3988. | AC005923 | Genbank | 4309927 |
| 3989. | AC005924 | Genbank | 4309926 |
| 3990. | AC005953 | Genbank | 3851204 |
| 3991. | AC005972 | Genbank | 3928123 |
| 3992. | AC005996 | Genbank | 4309892 |
| 3993. | AC006010 | Genbank | 4753277 |
| 3994. | AC006023 | Genbank | 4753260 |
| 3995. | AC006028 | Genbank | 7958982 |
| 3996. | AC006033 | Genbank | 4309948 |
| 3997. | AC006038 | Genbank | 7321960 |
| 3998. | AC006043 | Genbank | 4156139 |
| 3999. | AC006057 | Genbank | 4731048 |
| 4000. | AC006084 | Genbank | 3953485 |
| 4001. | AC006088 | Genbank | 4204701 |
| 4002. | AC006116 | Genbank | 3962497 |
| 4003. | AC006121 | Genbank | 4062176 |
| 4004. | AC006148 | Genbank | 4753275 |
| 4005. | AC006157 | Genbank | 4314424 |
| 4006. | AC006213 | Genbank | 4160143 |
| 4007. | AC006229 | Genbank | 6670859 |
| 4008. | AC006238 | Genbank | 4204704 |
| 4009. | AC006241 | Genbank | 4160141 |
| 4010. | AC006256 | Genbank | 4090190 |
| 4011. | AC006265 | Genbank | 4199962 |
| 4012. | AC006270 | Genbank | 4153853 |
| 4013. | AC006287 | Genbank | 4210508 |
| 4014. | AC006299 | Genbank | 4106997 |
| 4015. | AC006312 | Genbank | 4582483 |
| 4016. | AC006317 | Genbank | 4827327 |
| 4017. | AC006323 | Genbank | 4753259 |
| 4018. | AC006324 | Genbank | 6358867 |
| 4019. | AC006344 | Genbank | 4508150 |
| 4020. | AC006353 | Genbank | 4699957 |
| 4021. | AC006371 | Genbank | 5757526 |
| 4022. | AC006376 | Genbank | 4508126 |
| 4023. | AC006378 | Genbank | 4508123 |
| 4024. | AC006384 | Genbank | 4309808 |
| 4025. | AC006443 | Genbank | 4204325 |
| 4026. | AC006452 | Genbank | 9755473 |
| 4027. | AC006453 | Genbank | 4753285 |
| 4028. | AC006465 | Genbank | 4508124 |
| 4029. | AC006473 | Genbank | 11128437 |
| 4030. | AC006474 | Genbank | 4454627 |
| 4031. | AC006482 | Genbank | 6624076 |
| 4032. | AC006487 | Genbank | 11128438 |
| 4033. | AC006500 | Genbank | 4309875 |
| 4034. | AC006502 | Genbank | 4263368 |
| 4035. | AC006512 | Genbank | 4926863 |
| 4036. | AC006518 | Genbank | 4713939 |
| 4037. | AC006544 | Genbank | 4630756 |
| 4038. | AC006559 | Genbank | 4887253 |
| 4039. | AC006576 | Genbank | 4827300 |
| 4040. | AC006966 | Genbank | 5091657 |
| 4041. | AC006977 | Genbank | 5931479 |
| 4042. | AC006991 | Genbank | 10801457 |
| 4043. | AC007015 | Genbank | 4371262 |
| 4044. | AC007028 | Genbank | 6693583 |
| 4045. | AC007032 | Genbank | 5523832 |
| 4046. | AC007038 | Genbank | 5708475 |
| 4047. | AC007041 | Genbank | 5708471 |
| 4048. | AC007065 | Genbank | 4510439 |
| 4049. | AC007066 | Genbank | 4508098 |
| 4050. | AC007096 | Genbank | 5757525 |
| 4051. | AC007115 | Genbank | 4895146 |
| 4052. | AC007157 | Genbank | 4646258 |
| 4053. | AC007191 | Genbank | 4558643 |
| 4054. | AC007199 | Genbank | 4558768 |
| 4055. | AC007204 | Genbank | 4559317 |
| 4056. | AC007246 | Genbank | 7656638 |
| 4057. | AC007269 | Genbank | 5732139 |
| 4058. | AC007286 | Genbank | 5819124 |
| 4059. | AC007297 | Genbank | 5080744 |
| 4060. | AC007308 | Genbank | 5903110 |
| 4061. | AC007316 | Genbank | 8469003 |
| 4062. | AC007321 | Genbank | 7243928 |
| 4063. | AC007322 | Genbank | 9053584 |
| 4064. | AC007347 | Genbank | 6806840 |
| 4065. | AC007384 | Genbank | 5708473 |
| 4066. | AC007386 | Genbank | 5649377 |
| 4067. | AC007388 | Genbank | 5931452 |
| 4068. | AC007401 | Genbank | 10440760 |
| 4069. | AC007406 | Genbank | 4689442 |
| 4070. | AC007436 | Genbank | 4726097 |
| 4071. | AC007447 | Genbank | 4982552 |
| 4072. | AC007465 | Genbank | 10801482 |
| 4073. | AC007488 | Genbank | 5656676 |
| 4074. | AC007546 | Genbank | 5668756 |
| 4075. | AC007566 | Genbank | 11181861 |
| 4076. | AC007642 | Genbank | 9454611 |
| 4077. | AC007655 | Genbank | 4895153 |
| 4078. | AC007684 | Genbank | 5836173 |
| 4079. | AC007688 | Genbank | 5815499 |
| 4080. | AC007690 | Genbank | 5815494 |
| 4081. | AC007738 | Genbank | 6094647 |
| 4082. | AC007739 | Genbank | 7534255 |
| 4083. | AC007744 | Genbank | 7243926 |
| 4084. | AC007751 | Genbank | 5001551 |
| 4085. | AC007842 | Genbank | 5080755 |
| 4086. | AC007845 | Genbank | 7109495 |
| 4087. | AC007860 | Genbank | 5649179 |
| 4088. | AC007969 | Genbank | 7534288 |
| 4089. | AC008013 | Genbank | 5801645 |
| 4090. | AC008039 | Genbank | 5454236 |
| 4091. | AC008041 | Genbank | 5649181 |
| 4092. | AC008056 | Genbank | 6456144 |
| 4093. | AC008062 | Genbank | 5931476 |
| 4094. | AC008068 | Genbank | 8748885 |
| 4095. | AC008115 | Genbank | 5801654 |
| 4096. | AC008116 | Genbank | 6001959 |
| 4097. | AC008123 | Genbank | 6006046 |
| 4098. | AC008149 | Genbank | 6630488 |
| 4099. | AC008151 | Genbank | 5629925 |
| 4100. | AC008169 | Genbank | 6587935 |
| 4101. | AC008174 | Genbank | 6624082 |
| 4102. | AC008178 | Genbank | 9795977 |
| 4103. | AC008269 | Genbank | 10716637 |
| 4104. | AC008273 | Genbank | 6042091 |
| 4105. | AC008277 | Genbank | 10716648 |
| 4106. | AC008278 | Genbank | 7408138 |
| 4107. | AC008518 | Genbank | 7582547 |
| 4108. | AC008534 | Genbank | 9558573 |
| 4109. | AC008648 | Genbank | 9958009 |
| 4110. | AC008664 | Genbank | 9211191 |
| 4111. | AC008783 | Genbank | 7582549 |
| 4112. | AC008804 | Genbank | 9958011 |
| 4113. | AC008833 | Genbank | 7582550 |
| 4114. | AC008865 | Genbank | 7019284 |
| 4115. | AC008893 | Genbank | 7259691 |
| 4116. | AC008925 | Genbank | 7363399 |
| 4117. | AC008934 | Genbank | 7158896 |
| 4118. | AC008982 | Genbank | 9625322 |
| 4119. | AC009004 | Genbank | 9625323 |
| 4120. | AC009007 | Genbank | 7527772 |
| 4121. | AC009044 | Genbank | 6850298 |
| 4122. | AC009060 | Genbank | 9690317 |
| 4123. | AC009178 | Genbank | 6642684 |
| 4124. | AC009228 | Genbank | 10140829 |
| 4125. | AC009229 | Genbank | 10716650 |
| 4126. | AC009245 | Genbank | 8810492 |
| 4127. | AC009277 | Genbank | 9789644 |
| 4128. | AC009307 | Genbank | 9454630 |
| 4129. | AC009314 | Genbank | 8748928 |
| 4130. | AC009319 | Genbank | 9558561 |
| 4131. | AC009329 | Genbank | 8810493 |
| 4132. | AC009331 | Genbank | 9789636 |
| 4133. | AC009358 | Genbank | 9295771 |
| 4134. | AC009363 | Genbank | 6648145 |
| 4135. | AC009399 | Genbank | 6682219 |
| 4136. | AC009403 | Genbank | 10440729 |
| 4137. | AC009411 | Genbank | 6587929 |
| 4138. | AC009478 | Genbank | 7770675 |
| 4139. | AC009505 | Genbank | 8748933 |
| 4140. | AC009516 | Genbank | 6437562 |
| 4141. | AC009948 | Genbank | 7408084 |
| 4142. | AC009953 | Genbank | 8954248 |
| 4143. | AC009977 | Genbank | 10048082 |
| 4144. | AC010092 | Genbank | 8099095 |
| 4145. | AC010169 | Genbank | 6648139 |
| 4146. | AC010205 | Genbank | 6468049 |
| 4147. | AC010285 | Genbank | 7651784 |
| 4148. | AC010293 | Genbank | 10337626 |
| 4149. | AC010305 | Genbank | 7109393 |
| 4150. | AC010329 | Genbank | 7328724 |
| 4151. | AC010352 | Genbank | 7109394 |
| 4152. | AC010363 | Genbank | 9211203 |
| 4153. | AC010480 | Genbank | 10280744 |
| 4154. | AC010491 | Genbank | 7651783 |
| 4155. | AC010494 | Genbank | 7109400 |
| 4156. | AC010582 | Genbank | 6721135 |
| 4157. | AC010598 | Genbank | 9558579 |
| 4158. | AC010645 | Genbank | 7670119 |
| 4159. | AC010678 | Genbank | 10440740 |
| 4160. | AC010725 | Genbank | 10047972 |
| 4161. | AC010731 | Genbank | 9857582 |
| 4162. | AC010749 | Genbank | 7243954 |
| 4163. | AC011331 | Genbank | 9929686 |
| 4164. | AC011362 | Genbank | 6094545 |
| 4165. | AC011449 | Genbank | 7839902 |
| 4166. | AC011462 | Genbank | 7684381 |
| 4167. | AC011477 | Genbank | 9558580 |
| 4168. | AC011489 | Genbank | 9690320 |
| 4169. | AC011497 | Genbank | 8844110 |
| 4170. | AC011523 | Genbank | 8778953 |
| 4171. | AC011533 | Genbank | 8844111 |
| 4172. | AC011555 | Genbank | 9929689 |
| 4173. | AC011595 | Genbank | 7109320 |
| 4174. | AC011609 | Genbank | 7114193 |
| 4175. | AC011742 | Genbank | 9887800 |
| 4176. | AC011890 | Genbank | 7705211 |
| 4177. | AC012087 | Genbank | 7114465 |
| 4178. | AC012150 | Genbank | 9558560 |
| 4179. | AC012262 | Genbank | 7381642 |
| 4180. | AC012467 | Genbank | 7363385 |
| 4181. | AC012472 | Genbank | 7715629 |
| 4182. | AC013738 | Genbank | 8567747 |
| 4183. | AC015973 | Genbank | 9211403 |
| 4184. | AC016138 | Genbank | 7021552 |
| 4185. | AC016144 | Genbank | 10440579 |
| 4186. | AC016254 | Genbank | 9845100 |
| 4187. | AC016395 | Genbank | 9929646 |
| 4188. | AC016397 | Genbank | 8567750 |
| 4189. | AC016576 | Genbank | 10337630 |
| 4190. | AC016604 | Genbank | 9958015 |
| 4191. | AC017092 | Genbank | 9887823 |
| 4192. | AC018511 | Genbank | 7715630 |
| 4193. | AC018633 | Genbank | 6729063 |
| 4194. | AC018751 | Genbank | 8714562 |
| 4195. | AC018758 | Genbank | 9954648 |
| 4196. | AC018797 | Genbank | 8747372 |
| 4197. | AC018816 | Genbank | 9755439 |
| 4198. | AC019063 | Genbank | 8569822 |
| 4199. | AC019100 | Genbank | 9454638 |
| 4200. | AC019117 | Genbank | 8748931 |
| 4201. | AC019181 | Genbank | 8748861 |
| 4202. | AC019221 | Genbank | 10048073 |
| 4203. | AC020549 | Genbank | 9795680 |
| 4204. | AC020610 | Genbank | 7340292 |
| 4205. | AC020633 | Genbank | 7658301 |
| 4206. | AC020663 | Genbank | 6682593 |
| 4207. | AC020728 | Genbank | 7770044 |
| 4208. | AC020898 | Genbank | 10280746 |
| 4209. | AC020910 | Genbank | 9558584 |
| 4210. | AC021012 | Genbank | 10280932 |
| 4211. | AC021036 | Genbank | 9885981 |
| 4212. | AC021037 | Genbank | 9857500 |
| 4213. | AC021887 | Genbank | 9581979 |
| 4214. | AC022149 | Genbank | 7527774 |
| 4215. | AC022150 | Genbank | 8567765 |
| 4216. | AC022392 | Genbank | 9885980 |
| 4217. | AC022402 | Genbank | 8705033 |
| 4218. | AC022469 | Genbank | 9309504 |
| 4219. | AC022517 | Genbank | 6910562 |
| 4220. | AC022596 | Genbank | 10280853 |
| 4221. | AC023172 | Genbank | 6957691 |
| 4222. | AC023602 | Genbank | 7276296 |
| 4223. | AC024153 | Genbank | 10190646 |
| 4224. | AC024571 | Genbank | 10280747 |
| 4225. | AC026371 | Genbank | 9910029 |
| 4226. | AC026439 | Genbank | 9690324 |
| 4227. | AC026693 | Genbank | 10044353 |
| 4228. | AC027279 | Genbank | 8134857 |
| 4229. | AC027345 | Genbank | 10044360 |
| 4230. | AC037199 | Genbank | 7527775 |
| 4231. | AC058791 | Genbank | 8810490 |
| 4232. | AC062033 | Genbank | 9858445 |
| 4233. | AC067744 | Genbank | 10198303 |
| 4234. | AC069298 | Genbank | 9964951 |
| 4235. | AC078937 | Genbank | 9795656 |
| 4236. | AD000092 | Genbank | 1905905 |
| 4237. | AF001548 | Genbank | 2104552 |
| 4238. | AF004162 | Genbank | 3046385 |
| 4239. | AF005067 | Genbank | 6979018 |
| 4240. | AF005889 | Genbank | 2738491 |
| 4241. | AF006043 | Genbank | 2674061 |
| 4242. | AF007544 | Genbank | 2970122 |
| 4243. | AF015262 | Genbank | 2897092 |
| 4244. | AF015287 | Genbank | 4102714 |
| 4245. | AF015308 | Genbank | 2384716 |
| 4246. | AF015724 | Genbank | 2801427 |
| 4247. | AF017178 | Genbank | 4755084 |
| 4248. | AF017257 | Genbank | 2736086 |
| 4249. | AF020202 | Genbank | 2431999 |
| 4250. | AF023259 | Genbank | 3746337 |
| 4251. | AF025438 | Genbank | 2815603 |
| 4252. | AF027153 | Genbank | 2739093 |
| 4253. | AF029786 | Genbank | 3403166 |
| 4254. | AF035279 | Genbank | 2661028 |
| 4255. | AF035315 | Genbank | 2661077 |
| 4256. | AF036613 | Genbank | 2687639 |
| 4257. | AF037222 | Genbank | 2921499 |
| 4258. | AF038176 | Genbank | 2795896 |
| 4259. | AF038954 | Genbank | 3329377 |
| 4260. | AF039019 | Genbank | 2828109 |
| 4261. | AF039575 | Genbank | 2773157 |
| 4262. | AF039594 | Genbank | 3366667 |
| 4263. | AF039656 | Genbank | 2773159 |
| 4264. | AF040707 | Genbank | 3688794 |
| 4265. | AF041429 | Genbank | 6594626 |
| 4266. | AF044958 | Genbank | 4164447 |
| 4267. | AF044959 | Genbank | 3348136 |
| 4268. | AF047440 | Genbank | 3335135 |
| 4269. | AF050171 | Genbank | 5668577 |
| 4270. | AF052094 | Genbank | 3360400 |
| 4271. | AF052578 | Genbank | 2967847 |
| 4272. | AF052955 | Genbank | 8117711 |
| 4273. | AF054184 | Genbank | 4092055 |
| 4274. | AF054284 | Genbank | 4033734 |
| 4275. | AF055006 | Genbank | 3005726 |
| 4276. | AF055584 | Genbank | 3649607 |
| 4277. | AF060568 | Genbank | 4138921 |
| 4278. | AF061326 | Genbank | 4337461 |
| 4279. | AF064084 | Genbank | 3135668 |
| 4280. | AF067844 | Genbank | 4240386 |
| 4281. | AF069301 | Genbank | 3193335 |
| 4282. | AF070418 | Genbank | 3236451 |
| 4283. | AF070595 | Genbank | 3387972 |
| 4284. | AF070627 | Genbank | 3283894 |
| 4285. | AF070644 | Genbank | 3283917 |
| 4286. | AF070649 | Genbank | 3283923 |
| 4287. | AF070665 | Genbank | 4454705 |
| 4288. | AF070669 | Genbank | 4454713 |
| 4289. | AF071172 | Genbank | 5107833 |
| 4290. | AF071218 | Genbank | 9757713 |
| 4291. | AF072097 | Genbank | 5725511 |
| 4292. | AF073298 | Genbank | 3641537 |
| 4293. | AF073518 | Genbank | 3641541 |
| 4294. | AF073770 | Genbank | 5305442 |
| 4295. | AF077052 | Genbank | 4689151 |
| 4296. | AF077202 | Genbank | 4679017 |
| 4297. | AF078855 | Genbank | 5531824 |
| 4298. | AF080092 | Genbank | 4322311 |
| 4299. | AF084555 | Genbank | 5813858 |
| 4300. | AF085832 | Genbank | 3483146 |
| 4301. | AF086097 | Genbank | 3483442 |
| 4302. | AF086178 | Genbank | 3483523 |
| 4303. | AF086182 | Genbank | 3483527 |
| 4304. | AF086539 | Genbank | 3483884 |
| 4305. | AF091076 | Genbank | 3859989 |
| 4306. | AF092128 | Genbank | 5138905 |
| 4307. | AF092737 | Genbank | 4741762 |
| 4308. | AF097492 | Genbank | 6002670 |
| 4309. | AF098066 | Genbank | 6002668 |
| 4310. | AF100745 | Genbank | 5410275 |
| 4311. | AF101044 | Genbank | 4877591 |
| 4312. | AF103907 | Genbank | 6165973 |
| 4313. | AF104913 | Genbank | 3941723 |
| 4314. | AF105253 | Genbank | 7532779 |
| 4315. | AF106622 | Genbank | 4378528 |
| 4316. | AF106684 | Genbank | 5410333 |
| 4317. | AF107406 | Genbank | 5531905 |
| 4318. | AF107885 | Genbank | 3928926 |
| 4319. | AF109733 | Genbank | 4566529 |
| 4320. | AF110774 | Genbank | 6523794 |
| 4321. | AF113015 | Genbank | 6642753 |
| 4322. | AF113016 | Genbank | 6642755 |
| 4323. | AF116606 | Genbank | 7959715 |
| 4324. | AF116637 | Genbank | 7959775 |
| 4325. | AF116679 | Genbank | 7959856 |
| 4326. | AF116682 | Genbank | 7959862 |
| 4327. | AF116696 | Genbank | 7959890 |
| 4328. | AF117829 | Genbank | 4151947 |
| 4329. | AF119854 | Genbank | 7770144 |
| 4330. | AF119897 | Genbank | 7770230 |
| 4331. | AF119905 | Genbank | 7770246 |
| 4332. | AF124523 | Genbank | 4325309 |
| 4333. | AF127035 | Genbank | 5726288 |
| 4334. | AF128525 | Genbank | 4325379 |
| 4335. | AF128536 | Genbank | 5305705 |
| 4336. | AF131215 | Genbank | 8272457 |
| 4337. | AF131742 | Genbank | 4406554 |
| 4338. | AF131847 | Genbank | 4406688 |
| 4339. | AF132939 | Genbank | 4680648 |
| 4340. | AF134726 | Genbank | 4529886 |
| 4341. | AF140240 | Genbank | 7406867 |
| 4342. | AF141347 | Genbank | 4929133 |
| 4343. | AF143235 | Genbank | 6449465 |
| 4344. | AF147424 | Genbank | 4761775 |
| 4345. | AF151057 | Genbank | 7106835 |
| 4346. | AF151103 | Genbank | 5758136 |
| 4347. | AF151807 | Genbank | 4929566 |
| 4348. | AF151832 | Genbank | 4929616 |
| 4349. | AF151844 | Genbank | 4929640 |
| 4350. | AF151866 | Genbank | 4929684 |
| 4351. | AF151892 | Genbank | 4929736 |
| 4352. | AF151893 | Genbank | 4929738 |
| 4353. | AF151903 | Genbank | 4929758 |
| 4354. | AF152365 | Genbank | 5669135 |
| 4355. | AF152462 | Genbank | 5565976 |
| 4356. | AF153608 | Genbank | 5231140 |
| 4357. | AF155120 | Genbank | 6448866 |
| 4358. | AF157318 | Genbank | 7688686 |
| 4359. | AF161448 | Genbank | 6841309 |
| 4360. | AF161455 | Genbank | 6841323 |
| 4361. | AF161500 | Genbank | 6841523 |
| 4362. | AF163475 | Genbank | 7453521 |
| 4363. | AF164609 | Genbank | 5802809 |
| 4364. | AF164795 | Genbank | 8895092 |
| 4365. | AF165147 | Genbank | 5457160 |
| 4366. | AF166339 | Genbank | 5726557 |
| 4367. | AF172066 | Genbank | 5733811 |
| 4368. | AF175265 | Genbank | 9622849 |
| 4369. | AF178948 | Genbank | 8925845 |
| 4370. | AF179633 | Genbank | 5823550 |
| 4371. | AF186194 | Genbank | 5923927 |
| 4372. | AF186783 | Genbank | 10185670 |
| 4373. | AF187554 | Genbank | 6653225 |
| 4374. | AF187983 | Genbank | 6176320 |
| 4375. | AF191017 | Genbank | 6457337 |
| 4376. | AF191340 | Genbank | 6180014 |
| 4377. | AF196968 | Genbank | 6289084 |
| 4378. | AF200465 | Genbank | 6690789 |
| 4379. | AF201694 | Genbank | 9651485 |
| 4380. | AF201941 | Genbank | 9295185 |
| 4381. | AF203815 | Genbank | 6979641 |
| 4382. | AF207550 | Genbank | 6470333 |
| 4383. | AF208852 | Genbank | 7582291 |
| 4384. | AF208859 | Genbank | 7582305 |
| 4385. | AF212303 | Genbank | 8132761 |
| 4386. | AF217490 | Genbank | 8650408 |
| 4387. | AF217787 | Genbank | 7230513 |
| 4388. | AF225981 | Genbank | 7021496 |
| 4389. | AF227906 | Genbank | 7670747 |
| 4390. | AF231512 | Genbank | 10436080 |
| 4391. | AF235093 | Genbank | 7417261 |
| 4392. | AF247565 | Genbank | 7649252 |
| 4393. | AF250841 | Genbank | 8896064 |
| 4394. | AF253417 | Genbank | 8050709 |
| 4395. | AF261085 | Genbank | 9802301 |
| 4396. | AF262027 | Genbank | 9799079 |
| 4397. | AF266851 | Genbank | 9988622 |
| 4398. | AF272833 | Genbank | 8926325 |
| 4399. | AF283990 | Genbank | 10179949 |
| 4400. | AF284574 | Genbank | 9367115 |
| 4401. | AF285442 | Genbank | 9230789 |
| 4402. | AJ000644 | Genbank | 2695707 |
| 4403. | AJ005259 | Genbank | 3043444 |
| 4404. | AJ007509 | Genbank | 3319955 |
| 4405. | AJ010069 | Genbank | 3483012 |
| 4406. | AJ010071 | Genbank | 3483016 |
| 4407. | AJ010395 | Genbank | 5542013 |
| 4408. | AJ010442 | Genbank | 3954884 |
| 4409. | AJ010597 | Genbank | 3559873 |
| 4410. | AJ010952 | Genbank | 3646131 |
| 4411. | AJ011930 | Genbank | 3859769 |
| 4412. | AJ012159 | Genbank | 3805946 |
| 4413. | AJ012409 | Genbank | 3881975 |
| 4414. | AJ131016 | Genbank | 6911928 |
| 4415. | AJ132695 | Genbank | 8574037 |
| 4416. | AJ224112 | Genbank | 3413294 |
| 4417. | AJ225782 | Genbank | 4165269 |
| 4418. | AJ238093 | Genbank | 5725369 |
| 4419. | AJ239329 | Genbank | 5931933 |
| 4420. | AJ250044 | Genbank | 6469369 |
| 4421. | AJ251385 | Genbank | 9408099 |
| 4422. | AJ251760 | Genbank | 7362976 |
| 4423. | AJ270778 | Genbank | 9188427 |
| 4424. | AJ293624 | Genbank | 9650748 |
| 4425. | AJ295622 | Genbank | 9501304 |
| 4426. | AJ400877 | Genbank | 8052236 |
| 4427. | AK000009 | Genbank | 7019813 |
| 4428. | AK000049 | Genbank | 7019880 |
| 4429. | AK000061 | Genbank | 7019902 |
| 4430. | AK000095 | Genbank | 7019960 |
| 4431. | AK000121 | Genbank | 7020001 |
| 4432. | AK000269 | Genbank | 7020237 |
| 4433. | AK000341 | Genbank | 7020360 |
| 4434. | AK000426 | Genbank | 7020505 |
| 4435. | AK000432 | Genbank | 7020517 |
| 4436. | AK000628 | Genbank | 7020846 |
| 4437. | AK000667 | Genbank | 7020906 |
| 4438. | AK000685 | Genbank | 7020931 |
| 4439. | AK000709 | Genbank | 7020966 |
| 4440. | AK000800 | Genbank | 7021104 |
| 4441. | AK000882 | Genbank | 7021210 |
| 4442. | AK000959 | Genbank | 7021945 |
| 4443. | AK001061 | Genbank | 7022095 |
| 4444. | AK001138 | Genbank | 7022206 |
| 4445. | AK001143 | Genbank | 7022213 |
| 4446. | AK001313 | Genbank | 7022490 |
| 4447. | AK001335 | Genbank | 7022529 |
| 4448. | AK001441 | Genbank | 7022699 |
| 4449. | AK001443 | Genbank | 7022702 |
| 4450. | AK001445 | Genbank | 7022707 |
| 4451. | AK001451 | Genbank | 7022717 |
| 4452. | AK001569 | Genbank | 7022902 |
| 4453. | AK001768 | Genbank | 7023243 |
| 4454. | AK001805 | Genbank | 7023305 |
| 4455. | AK001885 | Genbank | 7023428 |
| 4456. | AK001950 | Genbank | 7023531 |
| 4457. | AK002150 | Genbank | 7023855 |
| 4458. | AK021895 | Genbank | 10433184 |
| 4459. | AK022100 | Genbank | 10433421 |
| 4460. | AK022204 | Genbank | 10433548 |
| 4461. | AK022503 | Genbank | 10433927 |
| 4462. | AK022855 | Genbank | 10434491 |
| 4463. | AK022868 | Genbank | 10434509 |
| 4464. | AK022993 | Genbank | 10434705 |
| 4465. | AK023096 | Genbank | 10434859 |
| 4466. | AK023161 | Genbank | 10434959 |
| 4467. | AK023284 | Genbank | 10435152 |
| 4468. | AK023329 | Genbank | 10435219 |
| 4469. | AK023455 | Genbank | 10435396 |
| 4470. | AK023535 | Genbank | 10435496 |
| 4471. | AK023562 | Genbank | 10435533 |
| 4472. | AK023673 | Genbank | 10435666 |
| 4473. | AK023985 | Genbank | 10436210 |
| 4474. | AK024077 | Genbank | 10436364 |
| 4475. | AK024090 | Genbank | 10436383 |
| 4476. | AK024129 | Genbank | 10436434 |
| 4477. | AK024146 | Genbank | 10436456 |
| 4478. | AK024327 | Genbank | 10436684 |
| 4479. | AK024436 | Genbank | 10440380 |
| 4480. | AK024692 | Genbank | 10437038 |
| 4481. | AK024888 | Genbank | 10437301 |
| 4482. | AK025120 | Genbank | 10437573 |
| 4483. | AK025317 | Genbank | 10437808 |
| 4484. | AK025375 | Genbank | 10437878 |
| 4485. | AK025588 | Genbank | 10438149 |
| 4486. | AK026110 | Genbank | 10438854 |
| 4487. | AK026132 | Genbank | 10438884 |
| 4488. | AK026164 | Genbank | 10438926 |
| 4489. | AK026515 | Genbank | 10439391 |
| 4490. | AK026568 | Genbank | 10439450 |
| 4491. | AK026569 | Genbank | 10439451 |
| 4492. | AK026683 | Genbank | 10439590 |
| 4493. | AK026933 | Genbank | 10439907 |
| 4494. | AK027003 | Genbank | 10440005 |
| 4495. | AL008627 | Genbank | 2769539 |
| 4496. | AL008631 | Genbank | 2760544 |
| 4497. | AL008633 | Genbank | 2578092 |
| 4498. | AL008639 | Genbank | 6599068 |
| 4499. | AL008987 | Genbank | 2695813 |
| 4500. | AL009029 | Genbank | 3135965 |
| 4501. | AL009051 | Genbank | 2995195 |
| 4502. | AL009181 | Genbank | 2853179 |
| 4503. | AL020990 | Genbank | 3980348 |
| 4504. | AL020995 | Genbank | 10862821 |
| 4505. | AL021026 | Genbank | 3171875 |
| 4506. | AL021069 | Genbank | 2853183 |
| 4507. | AL021368 | Genbank | 3080468 |
| 4508. | AL021807 | Genbank | 9367202 |
| 4509. | AL021937 | Genbank | 4165210 |
| 4510. | AL022069 | Genbank | 3256174 |
| 4511. | AL022100 | Genbank | 5881340 |
| 4512. | AL022144 | Genbank | 3421038 |
| 4513. | AL022147 | Genbank | 4808257 |
| 4514. | AL022152 | Genbank | 3150086 |
| 4515. | AL022238 | Genbank | 4176442 |
| 4516. | AL022240 | Genbank | 4826471 |
| 4517. | AL022313 | Genbank | 4200326 |
| 4518. | AL022316 | Genbank | 4691242 |
| 4519. | AL022326 | Genbank | 3550039 |
| 4520. | AL022329 | Genbank | 5002625 |
| 4521. | AL022333 | Genbank | 3281972 |
| 4522. | AL022393 | Genbank | 3046744 |
| 4523. | AL022395 | Genbank | 4468287 |
| 4524. | AL022476 | Genbank | 4493520 |
| 4525. | AL022722 | Genbank | 3367616 |
| 4526. | AL022727 | Genbank | 3093312 |
| 4527. | AL023284 | Genbank | 3355875 |
| 4528. | AL024497 | Genbank | 4680187 |
| 4529. | AL024498 | Genbank | 5924006 |
| 4530. | AL024508 | Genbank | 3445456 |
| 4531. | AL030996 | Genbank | 3688349 |
| 4532. | AL031005 | Genbank | 3287156 |
| 4533. | AL031055 | Genbank | 4375937 |
| 4534. | AL031177 | Genbank | 4071056 |
| 4535. | AL031183 | Genbank | 4468272 |
| 4536. | AL031276 | Genbank | 3947780 |
| 4537. | AL031277 | Genbank | 4375907 |
| 4538. | AL031281 | Genbank | 4826460 |
| 4539. | AL031282 | Genbank | 3860395 |
| 4540. | AL031293 | Genbank | 4140361 |
| 4541. | AL031297 | Genbank | 4902714 |
| 4542. | AL031315 | Genbank | 3820998 |
| 4543. | AL031320 | Genbank | 5457169 |
| 4544. | AL031431 | Genbank | 4938290 |
| 4545. | AL031432 | Genbank | 4375969 |
| 4546. | AL031433 | Genbank | 4826442 |
| 4547. | AL031655 | Genbank | 5360979 |
| 4548. | AL031665 | Genbank | 4826504 |
| 4549. | AL031670 | Genbank | 4469083 |
| 4550. | AL031673 | Genbank | 10198607 |
| 4551. | AL031677 | Genbank | 4464256 |
| 4552. | AL031714 | Genbank | 4775608 |
| 4553. | AL031730 | Genbank | 4090216 |
| 4554. | AL031777 | Genbank | 10198609 |
| 4555. | AL031847 | Genbank | 9369286 |
| 4556. | AL031905 | Genbank | 5918351 |
| 4557. | AL033378 | Genbank | 7159717 |
| 4558. | AL033392 | Genbank | 4826487 |
| 4559. | AL033531 | Genbank | 6807582 |
| 4560. | AL034344 | Genbank | 6624640 |
| 4561. | AL034375 | Genbank | 5763835 |
| 4562. | AL034379 | Genbank | 5918013 |
| 4563. | AL034395 | Genbank | 4581311 |
| 4564. | AL034417 | Genbank | 5102616 |
| 4565. | AL034423 | Genbank | 10198622 |
| 4566. | AL034452 | Genbank | 4678496 |
| 4567. | AL034551 | Genbank | 11125134 |
| 4568. | AL034553 | Genbank | 5419653 |
| 4569. | AL035079 | Genbank | 4775614 |
| 4570. | AL035209 | Genbank | 4160217 |
| 4571. | AL035411 | Genbank | 6136940 |
| 4572. | AL035420 | Genbank | 5918161 |
| 4573. | AL035448 | Genbank | 5830352 |
| 4574. | AL035461 | Genbank | 5123778 |
| 4575. | AL035465 | Genbank | 4581301 |
| 4576. | AL035466 | Genbank | 4775602 |
| 4577. | AL035534 | Genbank | 4455651 |
| 4578. | AL035610 | Genbank | 6249454 |
| 4579. | AL035670 | Genbank | 7838233 |
| 4580. | AL035681 | Genbank | 4902689 |
| 4581. | AL035686 | Genbank | 9795192 |
| 4582. | AL035689 | Genbank | 8218045 |
| 4583. | AL035693 | Genbank | 5817249 |
| 4584. | AL035701 | Genbank | 5679754 |
| 4585. | AL035705 | Genbank | 7159723 |
| 4586. | AL049378 | Genbank | 4500166 |
| 4587. | AL049386 | Genbank | 4500176 |
| 4588. | AL049450 | Genbank | 4500236 |
| 4589. | AL049471 | Genbank | 4500266 |
| 4590. | AL049552 | Genbank | 6010194 |
| 4591. | AL049563 | Genbank | 4902753 |
| 4592. | AL049564 | Genbank | 4902757 |
| 4593. | AL049565 | Genbank | 4826554 |
| 4594. | AL049715 | Genbank | 9581791 |
| 4595. | AL049742 | Genbank | 5419783 |
| 4596. | AL049748 | Genbank | 6572235 |
| 4597. | AL049757 | Genbank | 6002325 |
| 4598. | AL049758 | Genbank | 5263056 |
| 4599. | AL049776 | Genbank | 6681704 |
| 4600. | AL049792 | Genbank | 5763761 |
| 4601. | AL049823 | Genbank | 6273553 |
| 4602. | AL049830 | Genbank | 6729561 |
| 4603. | AL049835 | Genbank | 5708093 |
| 4604. | AL049838 | Genbank | 7248260 |
| 4605. | AL049839 | Genbank | 7009578 |
| 4606. | AL049840 | Genbank | 9368356 |
| 4607. | AL049849 | Genbank | 4826529 |
| 4608. | AL049856 | Genbank | 4826512 |
| 4609. | AL049859 | Genbank | 5706480 |
| 4610. | AL049871 | Genbank | 6523626 |
| 4611. | AL049872 | Genbank | 5708105 |
| 4612. | AL049911 | Genbank | 6114591 |
| 4613. | AL049951 | Genbank | 4884198 |
| 4614. | AL050254 | Genbank | 4886422 |
| 4615. | AL050269 | Genbank | 4886444 |
| 4616. | AL050290 | Genbank | 4886512 |
| 4617. | AL050311 | Genbank | 5679847 |
| 4618. | AL050341 | Genbank | 5791509 |
| 4619. | AL050343 | Genbank | 6137008 |
| 4620. | AL050349 | Genbank | 8655549 |
| 4621. | AL078463 | Genbank | 5777575 |
| 4622. | AL078472 | Genbank | 9886715 |
| 4623. | AL078477 | Genbank | 5419778 |
| 4624. | AL078581 | Genbank | 6967288 |
| 4625. | AL078590 | Genbank | 8218068 |
| 4626. | AL078612 | Genbank | 5419788 |
| 4627. | AL078645 | Genbank | 8218091 |
| 4628. | AL079341 | Genbank | 8655533 |
| 4629. | AL080089 | Genbank | 5262506 |
| 4630. | AL080121 | Genbank | 5262554 |
| 4631. | AL080210 | Genbank | 5262699 |
| 4632. | AL096755 | Genbank | 5420192 |
| 4633. | AL096774 | Genbank | 6465842 |
| 4634. | AL096814 | Genbank | 7159757 |
| 4635. | AL096818 | Genbank | 5911847 |
| 4636. | AL096819 | Genbank | 9650753 |
| 4637. | AL109678 | Genbank | 5689803 |
| 4638. | AL109758 | Genbank | 8217865 |
| 4639. | AL109797 | Genbank | 8249456 |
| 4640. | AL109824 | Genbank | 8218095 |
| 4641. | AL109829 | Genbank | 9581829 |
| 4642. | AL109865 | Genbank | 6911646 |
| 4643. | AL109921 | Genbank | 9588678 |
| 4644. | AL109922 | Genbank | 6434774 |
| 4645. | AL109928 | Genbank | 7981303 |
| 4646. | AL109930 | Genbank | 7838241 |
| 4647. | AL109939 | Genbank | 6523730 |
| 4648. | AL109942 | Genbank | 8894643 |
| 4649. | AL109943 | Genbank | 7242680 |
| 4650. | AL109944 | Genbank | 8218116 |
| 4651. | AL109964 | Genbank | 7288048 |
| 4652. | AL109983 | Genbank | 9368492 |
| 4653. | AL110100 | Genbank | 5777884 |
| 4654. | AL110115 | Genbank | 10120310 |
| 4655. | AL110183 | Genbank | 5817095 |
| 4656. | AL110212 | Genbank | 5817134 |
| 4657. | AL110502 | Genbank | 5830428 |
| 4658. | AL110505 | Genbank | 6433827 |
| 4659. | AL117339 | Genbank | 6624931 |
| 4660. | AL117340 | Genbank | 6002147 |
| 4661. | AL117350 | Genbank | 7159817 |
| 4662. | AL117352 | Genbank | 6911681 |
| 4663. | AL117354 | Genbank | 6822199 |
| 4664. | AL117412 | Genbank | 5912102 |
| 4665. | AL117558 | Genbank | 5912097 |
| 4666. | AL117596 | Genbank | 5912164 |
| 4667. | AL117599 | Genbank | 5912167 |
| 4668. | AL117645 | Genbank | 5912235 |
| 4669. | AL118499 | Genbank | 8546815 |
| 4670. | AL118523 | Genbank | 9795217 |
| 4671. | AL118556 | Genbank | 8346742 |
| 4672. | AL121583 | Genbank | 9588417 |
| 4673. | AL121601 | Genbank | 7159760 |
| 4674. | AL121603 | Genbank | 6434634 |
| 4675. | AL121656 | Genbank | 7159617 |
| 4676. | AL121713 | Genbank | 7330680 |
| 4677. | AL121716 | Genbank | 7159797 |
| 4678. | AL121755 | Genbank | 7406634 |
| 4679. | AL121790 | Genbank | 8919824 |
| 4680. | AL121808 | Genbank | 7105860 |
| 4681. | AL121809 | Genbank | 8018146 |
| 4682. | AL121820 | Genbank | 8176917 |
| 4683. | AL121903 | Genbank | 7330682 |
| 4684. | AL121914 | Genbank | 9864672 |
| 4685. | AL121918 | Genbank | 8388653 |
| 4686. | AL121928 | Genbank | 8247025 |
| 4687. | AL121953 | Genbank | 9944244 |
| 4688. | AL121981 | Genbank | 9931107 |
| 4689. | AL121984 | Genbank | 8218063 |
| 4690. | AL122001 | Genbank | 9588441 |
| 4691. | AL122003 | Genbank | 6580480 |
| 4692. | AL122125 | Genbank | 8574298 |
| 4693. | AL132656 | Genbank | 8439394 |
| 4694. | AL132657 | Genbank | 8388638 |
| 4695. | AL132671 | Genbank | 9885189 |
| 4696. | AL132777 | Genbank | 6562026 |
| 4697. | AL132986 | Genbank | 7159620 |
| 4698. | AL133238 | Genbank | 6624577 |
| 4699. | AL133241 | Genbank | 7630038 |
| 4700. | AL133247 | Genbank | 6491719 |
| 4701. | AL133255 | Genbank | 8894156 |
| 4702. | AL133269 | Genbank | 9662888 |
| 4703. | AL133281 | Genbank | 8217430 |
| 4704. | AL133294 | Genbank | 9650513 |
| 4705. | AL133340 | Genbank | 7330646 |
| 4706. | AL133353 | Genbank | 6706037 |
| 4707. | AL133367 | Genbank | 7708222 |
| 4708. | AL133371 | Genbank | 6634066 |
| 4709. | AL133395 | Genbank | 10045255 |
| 4710. | AL133415 | Genbank | 7160477 |
| 4711. | AL133502 | Genbank | 8217902 |
| 4712. | AL133514 | Genbank | 7406359 |
| 4713. | AL133548 | Genbank | 7899142 |
| 4714. | AL133552 | Genbank | 10185399 |
| 4715. | AL133593 | Genbank | 6599181 |
| 4716. | AL135744 | Genbank | 6682291 |
| 4717. | AL135786 | Genbank | 8894169 |
| 4718. | AL135787 | Genbank | 9588110 |
| 4719. | AL135928 | Genbank | 9863460 |
| 4720. | AL135959 | Genbank | 6635874 |
| 4721. | AL135961 | Genbank | 8452636 |
| 4722. | AL135978 | Genbank | 7452884 |
| 4723. | AL136000 | Genbank | 7940101 |
| 4724. | AL136083 | Genbank | 8247507 |
| 4725. | AL136097 | Genbank | 7799632 |
| 4726. | AL136169 | Genbank | 7160488 |
| 4727. | AL136296 | Genbank | 8217913 |
| 4728. | AL136460 | Genbank | 9650519 |
| 4729. | AL136461 | Genbank | 7939218 |
| 4730. | AL136504 | Genbank | 7018294 |
| 4731. | AL136973 | Genbank | 9650520 |
| 4732. | AL136980 | Genbank | 8218328 |
| 4733. | AL136985 | Genbank | 9542710 |
| 4734. | AL137000 | Genbank | 9944121 |
| 4735. | AL137020 | Genbank | 10086020 |
| 4736. | AL137073 | Genbank | 9863480 |
| 4737. | AL137141 | Genbank | 8217499 |
| 4738. | AL137302 | Genbank | 6807766 |
| 4739. | AL137440 | Genbank | 6808001 |
| 4740. | AL137628 | Genbank | 6808426 |
| 4741. | AL137721 | Genbank | 6808159 |
| 4742. | AL137795 | Genbank | 9187168 |
| 4743. | AL137798 | Genbank | 9187169 |
| 4744. | AL137818 | Genbank | 7009598 |
| 4745. | AL138735 | Genbank | 9662918 |
| 4746. | AL138758 | Genbank | 8670587 |
| 4747. | AL139021 | Genbank | 8919851 |
| 4748. | AL139099 | Genbank | 8217934 |
| 4749. | AL139120 | Genbank | 7739105 |
| 4750. | AL139123 | Genbank | 10178428 |
| 4751. | AL139289 | Genbank | 7529223 |
| 4752. | AL139316 | Genbank | 8217940 |
| 4753. | AL139395 | Genbank | 7960578 |
| 4754. | AL157361 | Genbank | 7899161 |
| 4755. | AL157455 | Genbank | 7018470 |
| 4756. | AL157773 | Genbank | 9944137 |
| 4757. | AL157792 | Genbank | 7327842 |
| 4758. | AL157837 | Genbank | 9801319 |
| 4759. | AL157838 | Genbank | 9588158 |
| 4760. | AL157952 | Genbank | 8894241 |
| 4761. | AL158040 | Genbank | 8745067 |
| 4762. | AL158093 | Genbank | 8546622 |
| 4763. | AL158172 | Genbank | 9801324 |
| 4764. | AL158801 | Genbank | 8574301 |
| 4765. | AL158832 | Genbank | 9944141 |
| 4766. | AL160236 | Genbank | 7708227 |
| 4767. | AL161420 | Genbank | 10443397 |
| 4768. | AL161628 | Genbank | 10129841 |
| 4769. | AL161644 | Genbank | 9794996 |
| 4770. | AL161670 | Genbank | 7523421 |
| 4771. | AL161725 | Genbank | 10045359 |
| 4772. | AL161747 | Genbank | 7619714 |
| 4773. | AL161952 | Genbank | 7328002 |
| 4774. | AL161991 | Genbank | 7328122 |
| 4775. | AL162047 | Genbank | 7328089 |
| 4776. | AL162049 | Genbank | 7328093 |
| 4777. | AL162294 | Genbank | 8218483 |
| 4778. | AL162390 | Genbank | 8894273 |
| 4779. | AL163151 | Genbank | 7452889 |
| 4780. | AL163279 | Genbank | 7717362 |
| 4781. | AL163284 | Genbank | 7717380 |
| 4782. | AL163301 | Genbank | 7717439 |
| 4783. | AL163302 | Genbank | 7717445 |
| 4784. | AL163541 | Genbank | 9581603 |
| 4785. | AL353759 | Genbank | 8745068 |
| 4786. | AL353771 | Genbank | 8894296 |
| 4787. | AL353950 | Genbank | 7669991 |
| 4788. | AL354720 | Genbank | 8979470 |
| 4789. | AL354766 | Genbank | 10185557 |
| 4790. | AL354772 | Genbank | 8217726 |
| 4791. | AL354773 | Genbank | 8894303 |
| 4792. | AL354915 | Genbank | 10129445 |
| 4793. | AL354984 | Genbank | 9988323 |
| 4794. | AL355094 | Genbank | 7799787 |
| 4795. | AL355708 | Genbank | 7799100 |
| 4796. | AL357153 | Genbank | 8919854 |
| 4797. | AL359332 | Genbank | 8573959 |
| 4798. | AL359402 | Genbank | 9186923 |
| 4799. | AL359600 | Genbank | 8655667 |
| 4800. | AL359763 | Genbank | 10045472 |
| 4801. | AL360190 | Genbank | 8919391 |
| 4802. | AL389887 | Genbank | 9944194 |
| 4803. | AL390755 | Genbank | 9864606 |
| 4804. | AP000009 | Genbank | 4666255 |
| 4805. | AP000010 | Genbank | 4666256 |
| 4806. | AP000012 | Genbank | 4666258 |
| 4807. | AP000021 | Genbank | 4666265 |
| 4808. | AP000022 | Genbank | 4666266 |
| 4809. | AP000038 | Genbank | 3132348 |
| 4810. | AP000045 | Genbank | 3132355 |
| 4811. | AP000049 | Genbank | 3132359 |
| 4812. | AP000069 | Genbank | 4579990 |
| 4813. | AP000075 | Genbank | 4579996 |
| 4814. | AP000079 | Genbank | 4580000 |
| 4815. | AP000086 | Genbank | 4730827 |
| 4816. | AP000087 | Genbank | 4730828 |
| 4817. | AP000089 | Genbank | 4730830 |
| 4818. | AP000097 | Genbank | 4730871 |
| 4819. | AP000131 | Genbank | 4730900 |
| 4820. | AP000173 | Genbank | 4827138 |
| 4821. | AP000433 | Genbank | 6177852 |
| 4822. | AP000497 | Genbank | 5926684 |
| 4823. | AP000526 | Genbank | 5931504 |
| 4824. | AP000948 | Genbank | 6691627 |
| 4825. | AP000953 | Genbank | 6693632 |
| 4826. | AP000962 | Genbank | 6942330 |
| 4827. | AP001053 | Genbank | 6693603 |
| 4828. | AP001137 | Genbank | 6970361 |
| 4829. | AP001207 | Genbank | 8698839 |
| 4830. | AP001330 | Genbank | 8698845 |
| 4831. | AP001343 | Genbank | 7209831 |
| 4832. | AP001597 | Genbank | 7670551 |
| 4833. | AP001598 | Genbank | 7670552 |
| 4834. | AP001603 | Genbank | 7670557 |
| 4835. | AP001605 | Genbank | 7670559 |
| 4836. | AP001619 | Genbank | 7670573 |
| 4837. | AP001631 | Genbank | 7670585 |
| 4838. | AX011717 | Genbank | 9998241 |
| 4839. | AX013090 | Genbank | 10040256 |
| 4840. | AX014092 | Genbank | 10040562 |
| 4841. | AX014108 | Genbank | 10040578 |
| 4842. | AX014211 | Genbank | 10040612 |
| 4843. | AX014313 | Genbank | 10040667 |
| 4844. | AX014326 | Genbank | 10040680 |
| 4845. | AX014332 | Genbank | 10040686 |
| 4846. | AX014337 | Genbank | 10040691 |
| 4847. | AX014868 | Genbank | 10041135 |
| 4848. | AX014878 | Genbank | 10041145 |
| 4849. | AX014884 | Genbank | 10041151 |
| 4850. | AX014886 | Genbank | 10041153 |
| 4851. | AX014898 | Genbank | 10041165 |
| 4852. | AX014914 | Genbank | 10041181 |
| 4853. | AX015387 | Genbank | 10041367 |
| 4854. | AX015422 | Genbank | 10041402 |
| 4855. | AX015525 | Genbank | 10041407 |
| 4856. | AX017261 | Genbank | 10042179 |
| 4857. | AX017518 | Genbank | 10042315 |
| 4858. | AY007110 | Genbank | 9956004 |
| 4859. | AY007135 | Genbank | 9956038 |
| 4860. | D00649 | Genbank | 219419 |
| 4861. | D00761 | Genbank | 220025 |
| 4862. | D00860 | Genbank | 220019 |
| 4863. | D10537 | Genbank | 220073 |
| 4864. | D14530 | Genbank | 414348 |
| 4865. | D14658 | Genbank | 285940 |
| 4866. | D14662 | Genbank | 285948 |
| 4867. | D14696 | Genbank | 285962 |
| 4868. | D14710 | Genbank | 559324 |
| 4869. | D16307 | Genbank | 303594 |
| 4870. | D16431 | Genbank | 598955 |
| 4871. | D16562 | Genbank | 506336 |
| 4872. | D21063 | Genbank | 434752 |
| 4873. | D26600 | Genbank | 565650 |
| 4874. | D29011 | Genbank | 558525 |
| 4875. | D29805 | Genbank | 474986 |
| 4876. | D29956 | Genbank | 473944 |
| 4877. | D31767 | Genbank | 505091 |
| 4878. | D32257 | Genbank | 1000446 |
| 4879. | D38047 | Genbank | 1037163 |
| 4880. | D38583 | Genbank | 560790 |
| 4881. | D43682 | Genbank | 1060913 |
| 4882. | D43717 | Genbank | 600558 |
| 4883. | D45248 | Genbank | 1008914 |
| 4884. | D49355 | Genbank | 1020405 |
| 4885. | D50544 | Genbank | 1345403 |
| 4886. | D50663 | Genbank | 1747307 |
| 4887. | D63875 | Genbank | 961441 |
| 4888. | D79205 | Genbank | 1754620 |
| 4889. | D83004 | Genbank | 1181557 |
| 4890. | D83770 | Genbank | 2388541 |
| 4891. | D86979 | Genbank | 6634000 |
| 4892. | D87127 | Genbank | 1817551 |
| 4893. | D87445 | Genbank | 6634006 |
| 4894. | D87666 | Genbank | 1620016 |
| 4895. | D89729 | Genbank | 2626839 |
| 4896. | E01497 | Genbank | 2169753 |
| 4897. | E02164 | Genbank | 2170402 |
| 4898. | E02628 | Genbank | 2170856 |
| 4899. | E02651 | Genbank | 2170879 |
| 4900. | E07218 | Genbank | 2175359 |
| 4901. | J02611 | Genbank | 178840 |
| 4902. | J03040 | Genbank | 338312 |
| 4903. | J03191 | Genbank | 190385 |
| 4904. | J04046 | Genbank | 179887 |
| 4905. | J04456 | Genbank | 187109 |
| 4906. | J04799 | Genbank | 190197 |
| 4907. | K01911 | Genbank | 189273 |
| 4908. | K03021 | Genbank | 339817 |
| 4909. | L05092 | Genbank | 388031 |
| 4910. | L05367 | Genbank | 189152 |
| 4911. | L09159 | Genbank | 307374 |
| 4912. | L11910 | Genbank | 292420 |
| 4913. | L13689 | Genbank | 291872 |
| 4914. | L13850 | Genbank | 292165 |
| 4915. | L25851 | Genbank | 4406707 |
| 4916. | L31610 | Genbank | 1220360 |
| 4917. | L32976 | Genbank | 488295 |
| 4918. | L34587 | Genbank | 551605 |
| 4919. | L34645 | Genbank | 598209 |
| 4920. | L38490 | Genbank | 601847 |
| 4921. | L39068 | Genbank | 994714 |
| 4922. | L41887 | Genbank | 950423 |
| 4923. | M10901 | Genbank | 183032 |
| 4924. | M11146 | Genbank | 182504 |
| 4925. | M11147 | Genbank | 182513 |
| 4926. | M11315 | Genbank | 180817 |
| 4927. | M11353 | Genbank | 184092 |
| 4928. | M14058 | Genbank | 179643 |
| 4929. | M14113 | Genbank | 182817 |
| 4930. | M15796 | Genbank | 181271 |
| 4931. | M15885 | Genbank | 338414 |
| 4932. | M16597 | Genbank | 181237 |
| 4933. | M19997 | Genbank | 181968 |
| 4934. | M20132 | Genbank | 178627 |
| 4935. | M21575 | Genbank | 1311702 |
| 4936. | M21895 | Genbank | 189523 |
| 4937. | M22414 | Genbank | 186260 |
| 4938. | M22918 | Genbank | 189019 |
| 4939. | M24194 | Genbank | 187701 |
| 4940. | M25160 | Genbank | 189059 |
| 4941. | M27024 | Genbank | 341598 |
| 4942. | M29929 | Genbank | 186471 |
| 4943. | M31606 | Genbank | 189940 |
| 4944. | M33216 | Genbank | 338234 |
| 4945. | M33651 | Genbank | 338060 |
| 4946. | M34840 | Genbank | 189620 |
| 4947. | M37510 | Genbank | 188897 |
| 4948. | M37583 | Genbank | 184059 |
| 4949. | M55643 | Genbank | 189179 |
| 4950. | M60457 | Genbank | 181249 |
| 4951. | M60828 | Genbank | 186738 |
| 4952. | M63391 | Genbank | 181539 |
| 4953. | M63573 | Genbank | 337998 |
| 4954. | M65181 | Genbank | 177128 |
| 4955. | M67480 | Genbank | 190364 |
| 4956. | M73554 | Genbank | 179364 |
| 4957. | M84526 | Genbank | 178625 |
| 4958. | S74678 | Genbank | 241477 |
| 4959. | S77127 | Genbank | 998582 |
| 4960. | S77512 | Genbank | 957311 |
| 4961. | S81752 | Genbank | 1438780 |
| 4962. | U03105 | Genbank | 476094 |
| 4963. | U04286 | Genbank | 460133 |
| 4964. | U07550 | Genbank | 469170 |
| 4965. | U07563 | Genbank | 514264 |
| 4966. | U10990 | Genbank | 758381 |
| 4967. | U12465 | Genbank | 562073 |
| 4968. | U12821 | Genbank | 642396 |
| 4969. | U13369 | Genbank | 555853 |
| 4970. | U14970 | Genbank | 550020 |
| 4971. | U14972 | Genbank | 550024 |
| 4972. | U15008 | Genbank | 600747 |
| 4973. | U17899 | Genbank | 717053 |
| 4974. | U17989 | Genbank | 805094 |
| 4975. | U21281 | Genbank | 732506 |
| 4976. | U23803 | Genbank | 773643 |
| 4977. | U24231 | Genbank | 809486 |
| 4978. | U24266 | Genbank | 1353247 |
| 4979. | U34605 | Genbank | 1144510 |
| 4980. | U38784 | Genbank | 1574947 |
| 4981. | U40763 | Genbank | 1117967 |
| 4982. | U41668 | Genbank | 1477481 |
| 4983. | U42412 | Genbank | 1335855 |
| 4984. | U49184 | Genbank | 1276978 |
| 4985. | U51561 | Genbank | 1236711 |
| 4986. | U54993 | Genbank | 4097314 |
| 4987. | U57847 | Genbank | 1373420 |
| 4988. | U61397 | Genbank | 1518693 |
| 4989. | U63630 | Genbank | 9800660 |
| 4990. | U64898 | Genbank | 2897866 |
| 4991. | U66871 | Genbank | 1519518 |
| 4992. | U68105 | Genbank | 1562509 |
| 4993. | U70734 | Genbank | 2360942 |
| 4994. | U73634 | Genbank | 1737189 |
| 4995. | U77085 | Genbank | 1684789 |
| 4996. | U78305 | Genbank | 2218062 |
| 4997. | U80763 | Genbank | 2231366 |
| 4998. | U81834 | Genbank | 1764141 |
| 4999. | U82696 | Genbank | 2735037 |
| 5000. | U83246 | Genbank | 1791256 |
| 5001. | U85193 | Genbank | 1814408 |
| 5002. | U87460 | Genbank | 2076881 |
| 5003. | U90304 | Genbank | 1899219 |
| 5004. | U90920 | Genbank | 2522321 |
| 5005. | U91321 | Genbank | 2951946 |
| 5006. | U91323 | Genbank | 3582311 |
| 5007. | U91324 | Genbank | 2339958 |
| 5008. | U95626 | Genbank | 2104517 |
| 5009. | X03963 | Genbank | 30091 |
| 5010. | X04098 | Genbank | 28338 |
| 5011. | X05278 | Genbank | 37201 |
| 5012. | X05332 | Genbank | 35740 |
| 5013. | X05610 | Genbank | 29550 |
| 5014. | X06747 | Genbank | 36101 |
| 5015. | X12791 | Genbank | 36112 |
| 5016. | X13238 | Genbank | 1200056 |
| 5017. | X13923 | Genbank | 30294 |
| 5018. | X14787 | Genbank | 37464 |
| 5019. | X15822 | Genbank | 30146 |
| 5020. | X15949 | Genbank | 33966 |
| 5021. | X16064 | Genbank | 37495 |
| 5022. | X16940 | Genbank | 36502 |
| 5023. | X51408 | Genbank | 35012 |
| 5024. | X52426 | Genbank | 30376 |
| 5025. | X52882 | Genbank | 311380 |
| 5026. | X52943 | Genbank | 28912 |
| 5027. | X54941 | Genbank | 29976 |
| 5028. | X55733 | Genbank | 288099 |
| 5029. | X56932 | Genbank | 23690 |
| 5030. | X56998 | Genbank | 37564 |
| 5031. | X57352 | Genbank | 311374 |
| 5032. | X63564 | Genbank | 36123 |
| 5033. | X67951 | Genbank | 287640 |
| 5034. | X70326 | Genbank | 38434 |
| 5035. | X74979 | Genbank | 400462 |
| 5036. | X76302 | Genbank | 431952 |
| 5037. | X80692 | Genbank | 763112 |
| 5038. | X81109 | Genbank | 535057 |
| 5039. | X82877 | Genbank | 1617112 |
| 5040. | X86163 | Genbank | 1220163 |
| 5041. | X87241 | Genbank | 1107686 |
| 5042. | X90583 | Genbank | 1071680 |
| 5043. | X98259 | Genbank | 1770465 |
| 5044. | Y00345 | Genbank | 35569 |
| 5045. | Y00815 | Genbank | 34266 |
| 5046. | Y13286 | Genbank | 2853173 |
| 5047. | Y14155 | Genbank | 2251148 |
| 5048. | Y15286 | Genbank | 2584788 |
| 5049. | Y18314 | Genbank | 5457356 |
| 5050. | Z11692 | Genbank | 31107 |
| 5051. | Z14227 | Genbank | 296120 |
| 5052. | Z21852 | Genbank | 311617 |
| 5053. | Z25535 | Genbank | 406224 |
| 5054. | Z68269 | Genbank | 1124903 |
| 5055. | Z68284 | Genbank | 1130698 |
| 5056. | Z68870 | Genbank | 1164910 |
| 5057. | Z73965 | Genbank | 1370122 |
| 5058. | Z75889 | Genbank | 1430780 |
| 5059. | Z81308 | Genbank | 6562055 |
| 5060. | Z81370 | Genbank | 1657265 |
| 5061. | Z82198 | Genbank | 6572207 |
| 5062. | Z82901 | Genbank | 1669681 |
| 5063. | Z83745 | Genbank | 1754650 |
| 5064. | Z83839 | Genbank | 2769655 |
| 5065. | Z83843 | Genbank | 2578095 |
| 5066. | Z83844 | Genbank | 4467204 |
| 5067. | Z84465 | Genbank | 6016927 |
| 5068. | Z84478 | Genbank | 3171880 |
| 5069. | Z84487 | Genbank | 4493528 |
| 5070. | Z84814 | Genbank | 1834458 |
| 5071. | Z85986 | Genbank | 4034056 |
| 5072. | Z86063 | Genbank | 1945152 |
| 5073. | Z93021 | Genbank | 4539080 |
| 5074. | Z93930 | Genbank | 4775603 |
| 5075. | Z93931 | Genbank | 2326511 |
| 5076. | Z95126 | Genbank | 2342581 |
| 5077. | Z95331 | Genbank | 6572282 |
| 5078. | Z97056 | Genbank | 2832593 |
| 5079. | Z97200 | Genbank | 3115994 |
| 5080. | Z98048 | Genbank | 2582746 |
| 5081. | Z98200 | Genbank | 6002294 |
| 5082. | Z98742 | Genbank | 5679741 |
| 5083. | Z99496 | Genbank | 2780183 |
| 5084. | Z99572 | Genbank | 2769646 |
| 5085. | Z99758 | Genbank | 4775628 |
| 5086. | Z99774 | Genbank | 2632107 |
| 5087. | Z99943 | Genbank | 2887308 |
| 5088. | G54862 | Genbank | |
| 5089. | G48942 | Genbank | 4529602 |
| 5090. | G50324 | Genbank | 5221501 |
| 5091. | G61630 | Genbank | 6126799 |
| 5092. | AL078630 | Genbank | 5051393 |
| 5093. | G16997 | Genbank | 1214423 |
| 5094. | AL009272 | Genbank | 2664435 |
| 5095. | G42611 | Genbank | 4115897 |
| 5096. | AL158629 | Genbank | 7161231 |
| 5097. | G00315 | Genbank | 485173 |
| 5098. | AA001546 | EST | 1437011 |
| 5099. | AA001792 | EST | 1445606 |
| 5100. | AA004871 | EST | 1447688 |
| 5101. | AA005402 | EST | 1447864 |
| 5102. | AA007634 | EST | 1463620 |
| 5103. | AA013088 | EST | 1474124 |
| 5104. | AA016190 | EST | 1477228 |
| 5105. | AA016209 | EST | 1477322 |
| 5106. | AA017190 | EST | 1479373 |
| 5107. | AA022854 | EST | 1486934 |
| 5108. | AA025352 | EST | 1489320 |
| 5109. | AA026455 | EST | 1492355 |
| 5110. | AA026671 | EST | 1492524 |
| 5111. | AA026687 | EST | 1492486 |
| 5112. | AA026689 | EST | 1492488 |
| 5113. | AA027143 | EST | 1492812 |
| 5114. | AA027171 | EST | 1492589 |
| 5115. | AA027816 | EST | 1493903 |
| 5116. | AA029500 | EST | 1497112 |
| 5117. | AA029664 | EST | 1497067 |
| 5118. | AA032079 | EST | 1502042 |
| 5119. | AA032220 | EST | 1502182 |
| 5120. | AA035190 | EST | 1507371 |
| 5121. | AA035372 | EST | 1507172 |
| 5122. | AA037216 | EST | 1512395 |
| 5123. | AA040429 | EST | 1516707 |
| 5124. | AA043221 | EST | 1521076 |
| 5125. | AA043442 | EST | 1521298 |
| 5126. | AA044829 | EST | 1523032 |
| 5127. | AA045965 | EST | 1525886 |
| 5128. | AA046053 | EST | 1525965 |
| 5129. | AA053583 | EST | 1544571 |
| 5130. | AA054125 | EST | 1545048 |
| 5131. | AA054357 | EST | 1545301 |
| 5132. | AA054729 | EST | 1545674 |
| 5133. | AA055921 | EST | 1548260 |
| 5134. | AA057620 | EST | 1550455 |
| 5135. | AA062983 | EST | 1557635 |
| 5136. | AA070493 | EST | 1577880 |
| 5137. | AA071165 | EST | 1578526 |
| 5138. | AA074729 | EST | 1614736 |
| 5139. | AA078983 | EST | 1617875 |
| 5140. | AA079801 | EST | 1618684 |
| 5141. | AA083210 | EST | 1625267 |
| 5142. | AA085273 | EST | 1627348 |
| 5143. | AA086480 | EST | 1629477 |
| 5144. | AA088553 | EST | 1634058 |
| 5145. | AA090151 | EST | 1636667 |
| 5146. | AA090257 | EST | 1636749 |
| 5147. | AA102835 | EST | 1648689 |
| 5148. | AA113376 | EST | 1665216 |
| 5149. | AA114907 | EST | 1670119 |
| 5150. | AA115745 | EST | 1670758 |
| 5151. | AA122064 | EST | 1678083 |
| 5152. | AA122239 | EST | 1678540 |
| 5153. | AA126689 | EST | 1686226 |
| 5154. | AA126814 | EST | 1686314 |
| 5155. | AA127488 | EST | 1686760 |
| 5156. | AA131735 | EST | 1693242 |
| 5157. | AA132284 | EST | 1693772 |
| 5158. | AA132709 | EST | 1694217 |
| 5159. | AA134425 | EST | 1691918 |
| 5160. | AA134446 | EST | 1691939 |
| 5161. | AA135542 | EST | 1696591 |
| 5162. | AA135757 | EST | 1696769 |
| 5163. | AA146684 | EST | 1716058 |
| 5164. | AA148083 | EST | 1717499 |
| 5165. | AA151678 | EST | 1720233 |
| 5166. | AA156054 | EST | 1727679 |
| 5167. | AA156092 | EST | 1727708 |
| 5168. | AA156867 | EST | 1728482 |
| 5169. | AA159473 | EST | 1735032 |
| 5170. | AA160569 | EST | 1735937 |
| 5171. | AA164775 | EST | 1740999 |
| 5172. | AA165068 | EST | 1740296 |
| 5173. | AA165165 | EST | 1740393 |
| 5174. | AA169229 | EST | 1748269 |
| 5175. | AA173288 | EST | 1753420 |
| 5176. | AA179187 | EST | 1760556 |
| 5177. | AA179224 | EST | 1760576 |
| 5178. | AA181019 | EST | 1764221 |
| 5179. | AA181634 | EST | 1765101 |
| 5180. | AA182043 | EST | 1764517 |
| 5181. | AA191079 | EST | 1779671 |
| 5182. | AA195244 | EST | 1784944 |
| 5183. | AA196038 | EST | 1791629 |
| 5184. | AA197305 | EST | 1792914 |
| 5185. | AA211710 | EST | 1810514 |
| 5186. | AA214368 | EST | 1813004 |
| 5187. | AA223183 | EST | 1843726 |
| 5188. | AA229607 | EST | 1851604 |
| 5189. | AA234994 | EST | 1858126 |
| 5190. | AA244131 | EST | 1874833 |
| 5191. | AA247529 | EST | 1879234 |
| 5192. | AA252028 | EST | 1887008 |
| 5193. | AA259243 | EST | 1894695 |
| 5194. | AA261960 | EST | 1898149 |
| 5195. | AA278456 | EST | 1919829 |
| 5196. | AA278930 | EST | 1920396 |
| 5197. | AA280063 | EST | 1921537 |
| 5198. | AA280327 | EST | 1921865 |
| 5199. | AA281639 | EST | 1924318 |
| 5200. | AA282654 | EST | 1925625 |
| 5201. | AA282758 | EST | 1925692 |
| 5202. | AA284243 | EST | 1928543 |
| 5203. | AA284519 | EST | 1928828 |
| 5204. | AA287975 | EST | 1933991 |
| 5205. | AA306963 | EST | 1959313 |
| 5206. | AA308340 | EST | 1960689 |
| 5207. | AA313204 | EST | 1965563 |
| 5208. | AA316962 | EST | 1969310 |
| 5209. | AA318158 | EST | 1970685 |
| 5210. | AA327175 | EST | 1979422 |
| 5211. | AA331942 | EST | 1984184 |
| 5212. | AA360261 | EST | 2012619 |
| 5213. | AA398127 | EST | 2051236 |
| 5214. | AA403032 | EST | 2056769 |
| 5215. | AA406187 | EST | 2064213 |
| 5216. | AA416637 | EST | 2077571 |
| 5217. | AA416731 | EST | 2077666 |
| 5218. | AA417367 | EST | 2077677 |
| 5219. | AA418821 | EST | 2080622 |
| 5220. | AA419178 | EST | 2078925 |
| 5221. | AA420614 | EST | 2094586 |
| 5222. | AA421015 | EST | 2099848 |
| 5223. | AA421139 | EST | 2099964 |
| 5224. | AA421950 | EST | 2100766 |
| 5225. | AA428236 | EST | 2110137 |
| 5226. | AA428584 | EST | 2112581 |
| 5227. | AA429767 | EST | 2112940 |
| 5228. | AA430212 | EST | 2113385 |
| 5229. | AA432373 | EST | 2114756 |
| 5230. | AA435599 | EST | 2140513 |
| 5231. | AA436886 | EST | 2141800 |
| 5232. | AA437159 | EST | 2142073 |
| 5233. | AA442415 | EST | 2154293 |
| 5234. | AA443828 | EST | 2156503 |
| 5235. | AA443900 | EST | 2156575 |
| 5236. | AA445998 | EST | 2158663 |
| 5237. | AA447320 | EST | 3025406 |
| 5238. | AA448760 | EST | 2162430 |
| 5239. | AA449768 | EST | 2163518 |
| 5240. | AA450122 | EST | 2163872 |
| 5241. | AA452114 | EST | 2165783 |
| 5242. | AA452665 | EST | 2166334 |
| 5243. | AA455686 | EST | 2178462 |
| 5244. | AA456287 | EST | 2179497 |
| 5245. | AA463961 | EST | 2188845 |
| 5246. | AA464646 | EST | 2189530 |
| 5247. | AA464792 | EST | 2189676 |
| 5248. | AA464866 | EST | 2189750 |
| 5249. | AA477388 | EST | 2206022 |
| 5250. | AA480099 | EST | 2208250 |
| 5251. | AA481058 | EST | 2210610 |
| 5252. | AA481325 | EST | 2210877 |
| 5253. | AA483857 | EST | 2212670 |
| 5254. | AA486837 | EST | 2217001 |
| 5255. | AA494555 | EST | 2224342 |
| 5256. | AA506576 | EST | 2242331 |
| 5257. | AA513015 | EST | 2251438 |
| 5258. | AA513399 | EST | 2251811 |
| 5259. | AA513597 | EST | 2252009 |
| 5260. | AA515361 | EST | 2254961 |
| 5261. | AA516531 | EST | 2256055 |
| 5262. | AA521342 | EST | 2261885 |
| 5263. | AA523252 | EST | 2263964 |
| 5264. | AA527493 | EST | 2269562 |
| 5265. | AA528154 | EST | 2270223 |
| 5266. | AA533727 | EST | 2277743 |
| 5267. | AA534586 | EST | 2278839 |
| 5268. | AA534641 | EST | 2278894 |
| 5269. | AA535621 | EST | 2279874 |
| 5270. | AA541515 | EST | 2287949 |
| 5271. | AA551591 | EST | 2321843 |
| 5272. | AA552696 | EST | 2322950 |
| 5273. | AA552702 | EST | 2322956 |
| 5274. | AA554692 | EST | 2325231 |
| 5275. | AA564018 | EST | 2335657 |
| 5276. | AA570691 | EST | 2344671 |
| 5277. | AA573135 | EST | 2347663 |
| 5278. | AA574090 | EST | 2348605 |
| 5279. | AA574340 | EST | 2348855 |
| 5280. | AA582196 | EST | 2360874 |
| 5281. | AA582548 | EST | 2359908 |
| 5282. | AA595186 | EST | 2410536 |
| 5283. | AA595339 | EST | 2410689 |
| 5284. | AA609045 | EST | 2457473 |
| 5285. | AA610247 | EST | 2458675 |
| 5286. | AA625304 | EST | 2537689 |
| 5287. | AA629269 | EST | 2541656 |
| 5288. | AA631460 | EST | 2554071 |
| 5289. | AA631706 | EST | 2554317 |
| 5290. | AA635305 | EST | 2559147 |
| 5291. | AA640241 | EST | 2565491 |
| 5292. | AA641195 | EST | 2566445 |
| 5293. | AA643275 | EST | 2568493 |
| 5294. | AA654789 | EST | 2590943 |
| 5295. | AA659388 | EST | 2595542 |
| 5296. | AA662347 | EST | 2616438 |
| 5297. | AA662764 | EST | 2616755 |
| 5298. | AA677583 | EST | 2658105 |
| 5299. | AA678913 | EST | 2659435 |
| 5300. | AA680156 | EST | 2656623 |
| 5301. | AA682647 | EST | 2669928 |
| 5302. | AA687931 | EST | 2674837 |
| 5303. | AA688365 | EST | 2675271 |
| 5304. | AA689292 | EST | 2690536 |
| 5305. | AA701603 | EST | 2704768 |
| 5306. | AA703249 | EST | 2706362 |
| 5307. | AA704322 | EST | 2714240 |
| 5308. | AA707411 | EST | 2717329 |
| 5309. | AA713791 | EST | 2726065 |
| 5310. | AA720676 | EST | 2736811 |
| 5311. | AA723194 | EST | 2740971 |
| 5312. | AA736481 | EST | 2767715 |
| 5313. | AA740723 | EST | 2779315 |
| 5314. | AA743584 | EST | 2783090 |
| 5315. | AA744862 | EST | 2783626 |
| 5316. | AA745867 | EST | 2785853 |
| 5317. | AA748038 | EST | 2787996 |
| 5318. | AA748559 | EST | 2788517 |
| 5319. | AA767784 | EST | 2818799 |
| 5320. | AA770207 | EST | 2821445 |
| 5321. | AA777621 | EST | 2837100 |
| 5322. | AA777631 | EST | 2837110 |
| 5323. | AA778767 | EST | 2838098 |
| 5324. | AA804326 | EST | 2873613 |
| 5325. | AA811828 | EST | 2881439 |
| 5326. | AA812993 | EST | 2883057 |
| 5327. | AA813187 | EST | 2883172 |
| 5328. | AA833812 | EST | 2908580 |
| 5329. | AA837457 | EST | 2912656 |
| 5330. | AA843122 | EST | 2929640 |
| 5331. | AA845415 | EST | 2933174 |
| 5332. | AA846735 | EST | 2932875 |
| 5333. | AA856697 | EST | 2944999 |
| 5334. | AA885486 | EST | 2994563 |
| 5335. | AA886068 | EST | 3001176 |
| 5336. | AA889841 | EST | 3016720 |
| 5337. | AA910949 | EST | 3050239 |
| 5338. | AA917420 | EST | 3057310 |
| 5339. | AA918401 | EST | 3058291 |
| 5340. | AA928019 | EST | 3077175 |
| 5341. | AA931246 | EST | 3085632 |
| 5342. | AA932735 | EST | 3086700 |
| 5343. | AA935517 | EST | 3092674 |
| 5344. | AA947511 | EST | 3108764 |
| 5345. | AA969498 | EST | 3144678 |
| 5346. | AA987973 | EST | 3173337 |
| 5347. | AA999966 | EST | 3190521 |
| 5348. | AF001542 | EST | 2529714 |
| 5349. | AF150232 | EST | 5133668 |
| 5350. | AF150245 | EST | 5133681 |
| 5351. | AI002431 | EST | 3202765 |
| 5352. | AI004627 | EST | 3214137 |
| 5353. | AI014677 | EST | 3229058 |
| 5354. | AI022037 | EST | 3239390 |
| 5355. | AI028763 | EST | 3246072 |
| 5356. | AI034384 | EST | 3255337 |
| 5357. | AI052626 | EST | 3308617 |
| 5358. | AI064691 | EST | 6358963 |
| 5359. | AI074350 | EST | 3400994 |
| 5360. | AI078597 | EST | 3413005 |
| 5361. | AI085346 | EST | 3423769 |
| 5362. | AI092862 | EST | 3431838 |
| 5363. | AI122870 | EST | 3538636 |
| 5364. | AI132931 | EST | 6360247 |
| 5365. | AI132935 | EST | 6360251 |
| 5366. | AI139470 | EST | 3645442 |
| 5367. | AI140677 | EST | 3648134 |
| 5368. | AI140777 | EST | 3648234 |
| 5369. | AI141584 | EST | 3649041 |
| 5370. | AI143587 | EST | 3665396 |
| 5371. | AI148326 | EST | 3676008 |
| 5372. | AI160389 | EST | 3693769 |
| 5373. | AI174693 | EST | 6361071 |
| 5374. | AI188340 | EST | 3739549 |
| 5375. | AI206732 | EST | 3765404 |
| 5376. | AI207272 | EST | 3765944 |
| 5377. | AI214446 | EST | 3778047 |
| 5378. | AI215841 | EST | 3784882 |
| 5379. | AI224992 | EST | 3807705 |
| 5380. | AI239783 | EST | 3835180 |
| 5381. | AI241763 | EST | 3837160 |
| 5382. | AI246555 | EST | 3841952 |
| 5383. | AI262138 | EST | 3870341 |
| 5384. | AI267883 | EST | 3887050 |
| 5385. | AI268324 | EST | 3887491 |
| 5386. | AI276172 | EST | 3898446 |
| 5387. | AI277008 | EST | 3899276 |
| 5388. | AI279747 | EST | 3917981 |
| 5389. | AI280561 | EST | 3918794 |
| 5390. | AI281762 | EST | 3919995 |
| 5391. | AI282281 | EST | 3920514 |
| 5392. | AI284863 | EST | 3923096 |
| 5393. | AI292237 | EST | 3935011 |
| 5394. | AI298114 | EST | 3957850 |
| 5395. | AI298437 | EST | 3958173 |
| 5396. | AI299734 | EST | 3957648 |
| 5397. | AI301294 | EST | 3960640 |
| 5398. | AI306424 | EST | 3989495 |
| 5399. | AI307786 | EST | 4002390 |
| 5400. | AI346431 | EST | 4083637 |
| 5401. | AI350287 | EST | 4087493 |
| 5402. | AI356228 | EST | 4107849 |
| 5403. | AI357625 | EST | 4109246 |
| 5404. | AI361513 | EST | 4113134 |
| 5405. | AI362768 | EST | 4114378 |
| 5406. | AI364193 | EST | 4123882 |
| 5407. | AI369397 | EST | 4148150 |
| 5408. | AI373065 | EST | 4152931 |
| 5409. | AI374822 | EST | 4174812 |
| 5410. | AI375148 | EST | 4175138 |
| 5411. | AI376548 | EST | 4186397 |
| 5412. | AI378044 | EST | 4187897 |
| 5413. | AI379940 | EST | 4189793 |
| 5414. | AI418843 | EST | 4264774 |
| 5415. | AI420521 | EST | 4266452 |
| 5416. | AI433550 | EST | 4289963 |
| 5417. | AI436092 | EST | 4307765 |
| 5418. | AI452458 | EST | 4285752 |
| 5419. | AI458732 | EST | 4311311 |
| 5420. | AI473700 | EST | 4326745 |
| 5421. | AI475134 | EST | 4328179 |
| 5422. | AI478478 | EST | 4371704 |
| 5423. | AI479011 | EST | 4372179 |
| 5424. | AI492377 | EST | 4393380 |
| 5425. | AI554779 | EST | 4487142 |
| 5426. | AI557112 | EST | 4489475 |
| 5427. | AI559492 | EST | 4509697 |
| 5428. | AI564010 | EST | 4522467 |
| 5429. | AI573093 | EST | 4536467 |
| 5430. | AI608936 | EST | 4618103 |
| 5431. | AI613490 | EST | 4622657 |
| 5432. | AI625142 | EST | 4650073 |
| 5433. | AI625316 | EST | 4650247 |
| 5434. | AI628363 | EST | 4665163 |
| 5435. | AI632622 | EST | 4683952 |
| 5436. | AI634293 | EST | 4685623 |
| 5437. | AI635397 | EST | 4686727 |
| 5438. | AI638468 | EST | 4690702 |
| 5439. | AI658967 | EST | 4762537 |
| 5440. | AI659386 | EST | 4762956 |
| 5441. | AI660483 | EST | 4764053 |
| 5442. | AI660901 | EST | 4764484 |
| 5443. | AI684171 | EST | 4895465 |
| 5444. | AI685658 | EST | 4896952 |
| 5445. | AI689640 | EST | 4900934 |
| 5446. | AI697014 | EST | 4984914 |
| 5447. | AI740859 | EST | 5109147 |
| 5448. | AI750569 | EST | 5128833 |
| 5449. | AI753683 | EST | 5131947 |
| 5450. | AI761927 | EST | 5177594 |
| 5451. | AI769144 | EST | 5235653 |
| 5452. | AI803018 | EST | 5368490 |
| 5453. | AI810399 | EST | 5396965 |
| 5454. | AI825708 | EST | 5446379 |
| 5455. | AI825799 | EST | 5446470 |
| 5456. | AI863332 | EST | 5527439 |
| 5457. | AI865749 | EST | 5529856 |
| 5458. | AI872979 | EST | 5547028 |
| 5459. | AI879185 | EST | 5553234 |
| 5460. | AI885574 | EST | 5590738 |
| 5461. | AI909662 | EST | 6500342 |
| 5462. | AI914372 | EST | 5634227 |
| 5463. | AI928446 | EST | 5664410 |
| 5464. | AI933926 | EST | 5672796 |
| 5465. | AI936575 | EST | 5675445 |
| 5466. | AI969679 | EST | 5766497 |
| 5467. | AL035910 | EST | 5927622 |
| 5468. | AL039464 | EST | 5408512 |
| 5469. | AL040214 | EST | 5409178 |
| 5470. | AL043562 | EST | 5422949 |
| 5471. | AL043942 | EST | 5432170 |
| 5472. | AL046717 | EST | 5434780 |
| 5473. | AL047150 | EST | 5435180 |
| 5474. | AL119267 | EST | 5925166 |
| 5475. | AL121031 | EST | 5927032 |
| 5476. | AL135535 | EST | 6603722 |
| 5477. | AL138298 | EST | 6854979 |
| 5478. | AV648085 | EST | 9869099 |
| 5479. | AV648142 | EST | 9869156 |
| 5480. | AV648633 | EST | 9869647 |
| 5481. | AV648669 | EST | 9869683 |
| 5482. | AV651107 | EST | 9872121 |
| 5483. | AV683063 | EST | 10284926 |
| 5484. | AV686268 | EST | 10288131 |
| 5485. | AV690809 | EST | 10292672 |
| 5486. | AV697336 | EST | 10299199 |
| 5487. | AV700220 | EST | 10302191 |
| 5488. | AW009854 | EST | 5858632 |
| 5489. | AW014268 | EST | 5863025 |
| 5490. | AW063595 | EST | 8887532 |
| 5491. | AW075969 | EST | 6030967 |
| 5492. | AW082272 | EST | 6037424 |
| 5493. | AW103282 | EST | 6074017 |
| 5494. | AW129284 | EST | 6117228 |
| 5495. | AW160559 | EST | 6299660 |
| 5496. | AW169297 | EST | 6400822 |
| 5497. | AW169657 | EST | 6401182 |
| 5498. | AW173161 | EST | 6439109 |
| 5499. | AW173312 | EST | 6439260 |
| 5500. | AW183892 | EST | 6452406 |
| 5501. | AW188641 | EST | 6463001 |
| 5502. | AW241932 | EST | 6575686 |
| 5503. | AW249638 | EST | 6592631 |
| 5504. | AW250733 | EST | 6593726 |
| 5505. | AW274506 | EST | 6661536 |
| 5506. | AW276618 | EST | 6663648 |
| 5507. | AW277121 | EST | 6664151 |
| 5508. | AW292155 | EST | 6698791 |
| 5509. | AW293994 | EST | 6700630 |
| 5510. | AW301984 | EST | 6711661 |
| 5511. | AW327325 | EST | 6797820 |
| 5512. | AW362055 | EST | 6866705 |
| 5513. | AW362906 | EST | 6867556 |
| 5514. | AW364524 | EST | 6869174 |
| 5515. | AW390251 | EST | 6894910 |
| 5516. | AW390861 | EST | 6895520 |
| 5517. | AW418673 | EST | 6946556 |
| 5518. | AW468427 | EST | 7038533 |
| 5519. | AW470799 | EST | 7040905 |
| 5520. | AW500392 | EST | 7112961 |
| 5521. | AW503310 | EST | 7118578 |
| 5522. | AW571863 | EST | 7236594 |
| 5523. | AW572402 | EST | 7237135 |
| 5524. | AW578038 | EST | 7253087 |
| 5525. | AW581923 | EST | 7256972 |
| 5526. | AW591390 | EST | 7278635 |
| 5527. | AW629039 | EST | 7375829 |
| 5528. | AW749041 | EST | 7663973 |
| 5529. | AW771419 | EST | 7703474 |
| 5530. | AW780136 | EST | 7794739 |
| 5531. | AW802240 | EST | 7854110 |
| 5532. | AW813039 | EST | 7906033 |
| 5533. | AW813512 | EST | 7906506 |
| 5534. | AW816896 | EST | 7909890 |
| 5535. | AW819807 | EST | 7912801 |
| 5536. | AW827207 | EST | 7920904 |
| 5537. | AW835524 | EST | 7929498 |
| 5538. | AW845658 | EST | 7941175 |
| 5539. | AW850705 | EST | 7946222 |
| 5540. | AW854326 | EST | 7950019 |
| 5541. | AW856514 | EST | 7952207 |
| 5542. | AW862934 | EST | 7996953 |
| 5543. | AW880769 | EST | 8042779 |
| 5544. | AW892192 | EST | 8056306 |
| 5545. | AW896564 | EST | 8060769 |
| 5546. | AW950675 | EST | 8140333 |
| 5547. | AW951161 | EST | 8140828 |
| 5548. | AW951819 | EST | 8141498 |
| 5549. | AW952752 | EST | 8142435 |
| 5550. | AW953991 | EST | 8143569 |
| 5551. | AW954111 | EST | 8143794 |
| 5552. | AW954336 | EST | 8144019 |
| 5553. | AW960065 | EST | 8149749 |
| 5554. | AW961455 | EST | 8151139 |
| 5555. | AW962252 | EST | 8152051 |
| 5556. | AW963747 | EST | 8153583 |
| 5557. | AW964301 | EST | 8154137 |
| 5558. | AW964586 | EST | 8154422 |
| 5559. | AW965890 | EST | 8155726 |
| 5560. | AW974807 | EST | 8166010 |
| 5561. | AW975713 | EST | 8166931 |
| 5562. | AW977033 | EST | 8168273 |
| 5563. | AW979224 | EST | 8170512 |
| 5564. | AW994037 | EST | 8254233 |
| 5565. | AW999396 | EST | 8259630 |
| 5566. | BE003464 | EST | 8263697 |
| 5567. | BE005843 | EST | 8266076 |
| 5568. | BE044400 | EST | 8361453 |
| 5569. | BE045344 | EST | 8362397 |
| 5570. | BE046368 | EST | 8363421 |
| 5571. | BE065701 | EST | 8410351 |
| 5572. | BE069007 | EST | 8413657 |
| 5573. | BE093289 | EST | 8483741 |
| 5574. | BE152048 | EST | 8614769 |
| 5575. | BE161669 | EST | 8624390 |
| 5576. | BE222840 | EST | 8910158 |
| 5577. | BE258873 | EST | 9129368 |
| 5578. | BE267154 | EST | 9140742 |
| 5579. | BE269312 | EST | 9142930 |
| 5580. | BE273257 | EST | 9147766 |
| 5581. | BE326532 | EST | 9200308 |
| 5582. | BE348273 | EST | 9260126 |
| 5583. | BE379434 | EST | 9324799 |
| 5584. | BE379808 | EST | 9325173 |
| 5585. | BE390332 | EST | 9335697 |
| 5586. | BE390367 | EST | 9335732 |
| 5587. | BE467413 | EST | 9513188 |
| 5588. | BE467460 | EST | 9513235 |
| 5589. | BE501374 | EST | 9703782 |
| 5590. | BE503499 | EST | 9705907 |
| 5591. | BE536737 | EST | 9765382 |
| 5592. | BE551271 | EST | 9792963 |
| 5593. | BE551352 | EST | 9793044 |
| 5594. | BE560614 | EST | 9804334 |
| 5595. | BE562940 | EST | 9806660 |
| 5596. | BE567335 | EST | 9811055 |
| 5597. | BE621256 | EST | 9892194 |
| 5598. | BE676190 | EST | 10036731 |
| 5599. | BE676253 | EST | 10036794 |
| 5600. | BE695857 | EST | 10083017 |
| 5601. | BE707549 | EST | 10095814 |
| 5602. | BE741622 | EST | 10155614 |
| 5603. | BE744492 | EST | 10158484 |
| 5604. | BE745086 | EST | 10159078 |
| 5605. | BE780647 | EST | 10201845 |
| 5606. | BE782769 | EST | 10203967 |
| 5607. | BE828512 | EST | 10260890 |
| 5608. | BE834547 | EST | 10266925 |
| 5609. | BE839483 | EST | 10271861 |
| 5610. | BE859073 | EST | 10374770 |
| 5611. | BE872750 | EST | 10321526 |
| 5612. | BE873461 | EST | 10322237 |
| 5613. | BE876502 | EST | 10325278 |
| 5614. | BE879972 | EST | 10328748 |
| 5615. | BE881118 | EST | 10329894 |
| 5616. | BE882391 | EST | 10331167 |
| 5617. | BE905132 | EST | 10398108 |
| 5618. | F18858 | EST | 4825306 |
| 5619. | H04588 | EST | 867521 |
| 5620. | H06973 | EST | 870505 |
| 5621. | H09023 | EST | 873845 |
| 5622. | H54419 | EST | 994566 |
| 5623. | H66745 | EST | 1025485 |
| 5624. | H87301 | EST | 1068880 |
| 5625. | L44375 | EST | 1048812 |
| 5626. | N25954 | EST | 1140302 |
| 5627. | N29069 | EST | 1147305 |
| 5628. | N29320 | EST | 1147840 |
| 5629. | N30486 | EST | 1149006 |
| 5630. | N55484 | EST | 1198363 |
| 5631. | N58888 | EST | 1202778 |
| 5632. | N58953 | EST | 1202843 |
| 5633. | N63710 | EST | 1211539 |
| 5634. | R02531 | EST | 752267 |
| 5635. | R07749 | EST | 759672 |
| 5636. | T07054 | EST | 318203 |
| 5637. | T85745 | EST | 714097 |
| 5638. | T86669 | EST | 715021 |
| 5639. | T86761 | EST | 715113 |
| 5640. | W25904 | EST | 1306027 |
| 5641. | W28503 | EST | 1308658 |
| 5642. | W35202 | EST | 1317128 |
| 5643 | W80390 | EST | 1391407 |
| 5644. | W88882 | EST | 1404364 |
| 5645. | A01657 | NUCPATENT | 345215 |
| 5646. | T39279 | NUCPATENT | 647033 |
| 5647. | V40539 | NUCPATENT | N/A |
| 5648. | V59678 | NUCPATENT | N/A |
| 5649. | V82787 | NUCPATENT | N/A |
| 5650. | V88357 | NUCPATENT | N/A |
| 5651. | X35723 | NUCPATENT | N/A |
| 5652. | X52222 | NUCPATENT | 31213 |
| 5653. | X87149 | NUCPATENT | 1870187 |
| 5654. | X97841 | NUCPATENT | 1322145 |
| 5655. | Z00870 | NUCPATENT | N/A |
| 5656. | Z15917 | NUCPATENT | N/A |
| 5657. | Z97257 | NUCPATENT | 2245237 |
| 5658. | AC38551 | PREPATNUC | N/A |
| 5659. | AC44458 | PREPATNUC | N/A |
| 5660. | AC45686 | PREPATNUC | N/A |
| 5661. | A18757 | Genbank | 512463 |
| 5662. | A23013 | Genbank | 825583 |
| 5663. | A26221 | Genbank | 904883 |
| 5664. | A27284 | Genbank | 1247482 |
| 5665. | A31920 | Genbank | 1247569 |
| 5666. | AB000220 | Genbank | 3426162 |
| 5667. | AB000221 | Genbank | 2289718 |
| 5668. | AB000584 | Genbank | 1813326 |
| 5669. | AB000888 | Genbank | 2467297 |
| 5670. | AB001106 | Genbank | 1850083 |
| 5671. | AB002303 | Genbank | 2224550 |
| 5672. | AB002323 | Genbank | 2224590 |
| 5673. | AB002330 | Genbank | 2224604 |
| 5674. | AB002365 | Genbank | 2224674 |
| 5675. | AB002368 | Genbank | 2224680 |
| 5676. | AB002389 | Genbank | 2224722 |
| 5877. | AB002409 | Genbank | 2335034 |
| 5678. | AB002449 | Genbank | 2943812 |
| 5679. | AB002451 | Genbank | 2943817 |
| 5680. | AB002533 | Genbank | 1944124 |
| 5681. | AB003103 | Genbank | 1945610 |
| 5682. | AB005047 | Genbank | 3116213 |
| 5683. | AB006077 | Genbank | 2564010 |
| 5684. | AB006202 | Genbank | 2351036 |
| 5685. | AB006757 | Genbank | 2979421 |
| 5686. | AB007618 | Genbank | 2465177 |
| 5687. | AB007865 | Genbank | 2662090 |
| 5688. | AB007887 | Genbank | 2887428 |
| 5689. | AB007888 | Genbank | 2887430 |
| 5690. | AB007892 | Genbank | 2887434 |
| 5691. | AB007893 | Genbank | 2887436 |
| 5692. | AB007898 | Genbank | 2662156 |
| 5693. | AB007899 | Genbank | 2662158 |
| 5694. | AB007927 | Genbank | 3413877 |
| 5695. | AB007934 | Genbank | 3413891 |
| 5696. | AB007944 | Genbank | 3413911 |
| 5697. | AB007962 | Genbank | 3413936 |
| 5698. | AB007963 | Genbank | 3413937 |
| 5699. | AB007964 | Genbank | 3413939 |
| 5700. | AB008226 | Genbank | 3370992 |
| 5701. | AB009356 | Genbank | 2924623 |
| 5702. | AB009398 | Genbank | 3618342 |
| 5703. | AB011087 | Genbank | 3043553 |
| 5704. | AB011089 | Genbank | 3043557 |
| 5705. | AB011101 | Genbank | 3043581 |
| 5706. | AB011147 | Genbank | 3043673 |
| 5707. | AB011151 | Genbank | 3043681 |
| 5708. | AB011154 | Genbank | 3043687 |
| 5709. | AB011158 | Genbank | 3043695 |
| 5710. | AB011165 | Genbank | 3043709 |
| 5711. | AB011182 | Genbank | 3043743 |
| 5712. | AB011399 | Genbank | 3452571 |
| 5713. | AB012109 | Genbank | 6463665 |
| 5714. | AB012770 | Genbank | 3721579 |
| 5715. | AB013139 | Genbank | 3169124 |
| 5716. | AB013818 | Genbank | 6518430 |
| 5717. | AB014528 | Genbank | 3327069 |
| 5718. | AB014529 | Genbank | 3327071 |
| 5719. | AB014536 | Genbank | 3327085 |
| 5720. | AB014540 | Genbank | 3327093 |
| 5721. | AB014548 | Genbank | 3327109 |
| 5722. | AB014558 | Genbank | 3327129 |
| 5723. | AB014584 | Genbank | 6635132 |
| 5724. | AB014888 | Genbank | 3402484 |
| 5725. | AB015051 | Genbank | 3868937 |
| 5726. | AB015639 | Genbank | 5821139 |
| 5727. | AB015752 | Genbank | 3746106 |
| 5728. | AB016043 | Genbank | 4586865 |
| 5729. | AB016789 | Genbank | 4239882 |
| 5730. | AB017566 | Genbank | 4586379 |
| 5731. | AB017708 | Genbank | 3650434 |
| 5732. | AB018010 | Genbank | 5926733 |
| 5733. | AB018256 | Genbank | 3882146 |
| 5734. | AB018266 | Genbank | 3882166 |
| 5735. | AB018276 | Genbank | 3882186 |
| 5736. | AB018280 | Genbank | 3882194 |
| 5737. | AB018285 | Genbank | 3882204 |
| 5738. | AB018288 | Genbank | 3882210 |
| 5739. | AB018319 | Genbank | 3882272 |
| 5740. | AB018330 | Genbank | 3882294 |
| 5741. | AB018337 | Genbank | 3882308 |
| 5742. | AB018342 | Genbank | 3882318 |
| 5743. | AB018580 | Genbank | 6624210 |
| 5744. | AB019392 | Genbank | 4587122 |
| 5745. | AB019524 | Genbank | 4519939 |
| 5746. | AB019568 | Genbank | 3885371 |
| 5747. | AB020335 | Genbank | 6518494 |
| 5748. | AB020646 | Genbank | 4240166 |
| 5749. | AB020661 | Genbank | 4240196 |
| 5750. | AB020674 | Genbank | 4240222 |
| 5751. | AB020679 | Genbank | 4240232 |
| 5752. | AB020692 | Genbank | 4240258 |
| 5753. | AB020693 | Genbank | 4240260 |
| 5754. | AB020721 | Genbank | 4240316 |
| 5755. | AB020863 | Genbank | 4003383 |
| 5756. | AB020867 | Genbank | 4003387 |
| 5757. | AB021288 | Genbank | 4038732 |
| 5758. | AB022785 | Genbank | 4210446 |
| 5759. | AB023137 | Genbank | 4589471 |
| 5760. | AB023159 | Genbank | 4589527 |
| 5761. | AB023169 | Genbank | 4589547 |
| 5762. | AB023209 | Genbank | 4589627 |
| 5763. | AB023218 | Genbank | 4589645 |
| 5764. | AB023223 | Genbank | 4589655 |
| 5765. | AB023225 | Genbank | 4589659 |
| 5766. | AB023230 | Genbank | 4589675 |
| 5767. | AB024334 | Genbank | 6016837 |
| 5768. | AB026906 | Genbank | 5295993 |
| 5769. | AB027466 | Genbank | 6172220 |
| 5770. | AB028128 | Genbank | 8100053 |
| 5771. | AB028859 | Genbank | 6567165 |
| 5772. | AB028893 | Genbank | 6552364 |
| 5773. | AB029014 | Genbank | 5689518 |
| 5774. | AB029022 | Genbank | 5689534 |
| 5775. | AB029826 | Genbank | 9049351 |
| 5776. | AB031230 | Genbank | 8918521 |
| 5777. | AB032251 | Genbank | 6683491 |
| 5778. | AB032261 | Genbank | 7415720 |
| 5779. | AB032945 | Genbank | 6329707 |
| 5780. | AB032959 | Genbank | 6329832 |
| 5781. | AB032973 | Genbank | 6330032 |
| 5782. | AB032983 | Genbank | 6330128 |
| 5783. | AB032988 | Genbank | 6330164 |
| 5784. | AB033007 | Genbank | 6330242 |
| 5785. | AB033047 | Genbank | 6330603 |
| 5786. | AB033055 | Genbank | 6330699 |
| 5787. | AB033070 | Genbank | 6330818 |
| 5788. | AB033079 | Genbank | 6382025 |
| 5789. | AB033115 | Genbank | 6331419 |
| 5790. | AB034205 | Genbank | 6899845 |
| 5791. | AB034989 | Genbank | 9711684 |
| 5792. | AB037675 | Genbank | 9650961 |
| 5793. | AB037720 | Genbank | 7242952 |
| 5794. | AB037743 | Genbank | 7243024 |
| 5795. | AB037767 | Genbank | 7243072 |
| 5796. | AB037775 | Genbank | 7243088 |
| 5797. | AB037806 | Genbank | 7243150 |
| 5798. | AB037851 | Genbank | 7243240 |
| 5799. | AB037860 | Genbank | 7243275 |
| 5800. | AB040883 | Genbank | 7959158 |
| 5801. | AB040927 | Genbank | 7959248 |
| 5802. | AB040940 | Genbank | 7959274 |
| 5803. | AB040971 | Genbank | 7959342 |
| 5804. | AB042425 | Genbank | 7770094 |
| 5805. | AB043104 | Genbank | 8096475 |
| 5806. | AB046830 | Genbank | 10047294 |
| 5807. | AB046844 | Genbank | 10047324 |
| 5808. | AC000015 | Genbank | 4263638 |
| 5809. | AC000028 | Genbank | 11192143 |
| 5810. | AC000036 | Genbank | 7958979 |
| 5811. | AC000074 | Genbank | 7122733 |
| 5812. | AC000099 | Genbank | 1764159 |
| 5813. | AC000353 | Genbank | 6970735 |
| 5814. | AC000394 | Genbank | 2133911 |
| 5815. | AC000403 | Genbank | 2133864 |
| 5816. | AC001226 | Genbank | 2133862 |
| 5817. | AC001479 | Genbank | 1944795 |
| 5818. | AC002052 | Genbank | 7212008 |
| 5819. | AC002060 | Genbank | 7143420 |
| 5820. | AC002064 | Genbank | 2076723 |
| 5821. | AC002072 | Genbank | 2078473 |
| 5822. | AC002090 | Genbank | 2160130 |
| 5823. | AC002094 | Genbank | 2155224 |
| 5824. | AC002105 | Genbank | 2288973 |
| 5825. | AC002112 | Genbank | 2347181 |
| 5826. | AC002115 | Genbank | 2098573 |
| 5827. | AC002319 | Genbank | 2828782 |
| 5828. | AC002347 | Genbank | 2828783 |
| 5829. | AC002398 | Genbank | 2529398 |
| 5830. | AC002400 | Genbank | 2576344 |
| 5831. | AC002429 | Genbank | 2335067 |
| 5832. | AC002430 | Genbank | 2335066 |
| 5833. | AC002470 | Genbank | 6478944 |
| 5834. | AC002476 | Genbank | 2340101 |
| 5835. | AC002480 | Genbank | 2340098 |
| 5836. | AC002481 | Genbank | 2340092 |
| 5837. | AC002528 | Genbank | 2388554 |
| 5838. | AC002543 | Genbank | 3645947 |
| 5839. | AC002550 | Genbank | 2570261 |
| 5840. | AC002558 | Genbank | 2580474 |
| 5841. | AC003025 | Genbank | 3337308 |
| 5842. | AC003029 | Genbank | 9965520 |
| 5843. | AC003042 | Genbank | 3319121 |
| 5844. | AC003075 | Genbank | 2588637 |
| 5845. | AC003103 | Genbank | 2842782 |
| 5846. | AC003657 | Genbank | 2935593 |
| 5847. | AC003682 | Genbank | 3264845 |
| 5848. | AC003950 | Genbank | 4262275 |
| 5849. | AC003998 | Genbank | 2772568 |
| 5850. | AC004003 | Genbank | 2772557 |
| 5851. | AC004036 | Genbank | 2811104 |
| 5852. | AC004066 | Genbank | 4001527 |
| 5853. | AC004069 | Genbank | 3252802 |
| 5854. | AC004080 | Genbank | 2822164 |
| 5855. | AC004106 | Genbank | 3150276 |
| 5856. | AC004108 | Genbank | 3108047 |
| 5857. | AC004142 | Genbank | 2880078 |
| 5858. | AC004148 | Genbank | 3482960 |
| 5859. | AC004151 | Genbank | 2896795 |
| 5860. | AC004156 | Genbank | 2896799 |
| 5861. | AC004160 | Genbank | 2897862 |
| 5862. | AC004166 | Genbank | 8887011 |
| 5863. | AC004223 | Genbank | 3253129 |
| 5864. | AC004230 | Genbank | 4028938 |
| 5865. | AC004236 | Genbank | 2914668 |
| 5866. | AC004253 | Genbank | 3169206 |
| 5867. | AC004263 | Genbank | 2935616 |
| 5868. | AC004381 | Genbank | 2982169 |
| 5869. | AC004448 | Genbank | 11128431 |
| 5870. | AC004477 | Genbank | 3688107 |
| 5871. | AC004522 | Genbank | 3006227 |
| 5872. | AC004525 | Genbank | 3219333 |
| 5873. | AC004549 | Genbank | 3046306 |
| 5874. | AC004554 | Genbank | 3169141 |
| 5875. | AC004584 | Genbank | 3417305 |
| 5876. | AC004585 | Genbank | 3212882 |
| 5877. | AC004587 | Genbank | 3150014 |
| 5878. | AC004593 | Genbank | 3063518 |
| 5879. | AC004600 | Genbank | 4388746 |
| 5880. | AC004602 | Genbank | 3075375 |
| 5881. | AC004612 | Genbank | 3080666 |
| 5882. | AC004650 | Genbank | 3097834 |
| 5883. | AC004668 | Genbank | 3115345 |
| 5884. | AC004686 | Genbank | 3688105 |
| 5885. | AC004752 | Genbank | 3165402 |
| 5886. | AC004775 | Genbank | 3169300 |
| 5887. | AC004782 | Genbank | 3172145 |
| 5888. | AC004797 | Genbank | 3492891 |
| 5889. | AC004821 | Genbank | 11181836 |
| 5890. | AC004836 | Genbank | 4508152 |
| 5891. | AC004837 | Genbank | 3845417 |
| 5892. | AC004850 | Genbank | 4309890 |
| 5893. | AC004854 | Genbank | 4827328 |
| 5894. | AC004869 | Genbank | 4156194 |
| 5895. | AC004877 | Genbank | 3638954 |
| 5896. | AC004885 | Genbank | 4926909 |
| 5897. | AC004890 | Genbank | 4508146 |
| 5898. | AC004943 | Genbank | 3924671 |
| 5899. | AC004955 | Genbank | 4753267 |
| 5900. | AC004962 | Genbank | 4156163 |
| 5901. | AC004985 | Genbank | 5708490 |
| 5902. | AC004997 | Genbank | 5441941 |
| 5903. | AC005005 | Genbank | 4156153 |
| 5904. | AC005045 | Genbank | 4508117 |
| 5905. | AC005052 | Genbank | 10122134 |
| 5906. | AC005060 | Genbank | 10445386 |
| 5907. | AC005062 | Genbank | 4150931 |
| 5908. | AC005065 | Genbank | 4153861 |
| 5909. | AC005075 | Genbank | 4926889 |
| 5910. | AC005089 | Genbank | 5732140 |
| 5911. | AC005090 | Genbank | 3478661 |
| 5912. | AC005104 | Genbank | 4218027 |
| 5913. | AC005154 | Genbank | 3242763 |
| 5914. | AC005161 | Genbank | 3242754 |
| 5915. | AC005166 | Genbank | 3242769 |
| 5916. | AC005183 | Genbank | 4558524 |
| 5917. | AC005207 | Genbank | 3342221 |
| 5918. | AC005213 | Genbank | 3282167 |
| 5919. | AC005217 | Genbank | 3282163 |
| 5920. | AC005255 | Genbank | 3289998 |
| 5921. | AC005291 | Genbank | 3402737 |
| 5922. | AC005305 | Genbank | 3329352 |
| 5923. | AC005332 | Genbank | 3659494 |
| 5924. | AC005335 | Genbank | 3347824 |
| 5925. | AC005480 | Genbank | 4835818 |
| 5926. | AC005531 | Genbank | 4153875 |
| 5927. | AC005544 | Genbank | 3650045 |
| 5928. | AC005546 | Genbank | 3478635 |
| 5929. | AC005550 | Genbank | 3478668 |
| 5930. | AC005553 | Genbank | 4090193 |
| 5931. | AC005575 | Genbank | 3510226 |
| 5932. | AC005618 | Genbank | 3548785 |
| 5933. | AC005630 | Genbank | 4159882 |
| 5934. | AC005678 | Genbank | 4156137 |
| 5935. | AC005703 | Genbank | 7740042 |
| 5936. | AC005722 | Genbank | 3738108 |
| 5937. | AC005726 | Genbank | 3810672 |
| 5938. | AC005758 | Genbank | 3688087 |
| 5939. | AC005768 | Genbank | 6598827 |
| 5940. | AC005799 | Genbank | 3789715 |
| 5941. | AC005837 | Genbank | 3849820 |
| 5942. | AC005839 | Genbank | 4079626 |
| 5943. | AC005841 | Genbank | 4731044 |
| 5944. | AC005874 | Genbank | 6249674 |
| 5945. | AC005876 | Genbank | 6249672 |
| 5946. | AC005907 | Genbank | 4204700 |
| 5947. | AC005911 | Genbank | 6139058 |
| 5948. | AC005912 | Genbank | 4165005 |
| 5949. | AC005924 | Genbank | 4309926 |
| 5950. | AC005940 | Genbank | 4454511 |
| 5951. | AC005972 | Genbank | 3928123 |
| 5952. | AC005995 | Genbank | 9857562 |
| 5953. | AC005996 | Genbank | 4309892 |
| 5954. | AC006019 | Genbank | 4309959 |
| 5955. | AC006020 | Genbank | 4827323 |
| 5956. | AC006032 | Genbank | 4309957 |
| 5957. | AC006033 | Genbank | 4309948 |
| 5958. | AC006040 | Genbank | 10801470 |
| 5959. | AC006057 | Genbank | 4731048 |
| 5960. | AC006059 | Genbank | 4544348 |
| 5961. | AC006064 | Genbank | 4572650 |
| 5962. | AC006084 | Genbank | 3953485 |
| 5963. | AC006138 | Genbank | 3970958 |
| 5964. | AC006146 | Genbank | 5836157 |
| 5965. | AC006211 | Genbank | 4071020 |
| 5966. | AC006238 | Genbank | 4204704 |
| 5967. | AC006251 | Genbank | 4309945 |
| 5968. | AC006265 | Genbank | 4199962 |
| 5969. | AC006299 | Genbank | 4106997 |
| 5970. | AC006312 | Genbank | 4582483 |
| 5971. | AC006359 | Genbank | 4753263 |
| 5972. | AC006362 | Genbank | 5732137 |
| 5973. | AC006366 | Genbank | 7684534 |
| 5974. | AC006388 | Genbank | 4753219 |
| 5975. | AC006441 | Genbank | 4760435 |
| 5976. | AC006455 | Genbank | 5103909 |
| 5977. | AC006460 | Genbank | 7243904 |
| 5978. | AC006479 | Genbank | 6358868 |
| 5979. | AC006480 | Genbank | 6289252 |
| 5980. | AC006483 | Genbank | 9211523 |
| 5981. | AC006487 | Genbank | 11128438 |
| 5982. | AC006501 | Genbank | 4309874 |
| 5983. | AC006504 | Genbank | 4220442 |
| 5984. | AC006536 | Genbank | 4235135 |
| 5985. | AC006559 | Genbank | 4887253 |
| 5986. | AC006561 | Genbank | 4558534 |
| 5987. | AC006581 | Genbank | 4914350 |
| 5988. | AC006599 | Genbank | 4454485 |
| 5989. | AC006840 | Genbank | 6227027 |
| 5990. | AC006948 | Genbank | 4689496 |
| 5991. | AC007027 | Genbank | 7574876 |
| 5992. | AC007038 | Genbank | 5708475 |
| 5993. | AC007041 | Genbank | 5708471 |
| 5994. | AC007051 | Genbank | 4432877 |
| 5995. | AC007073 | Genbank | 4416547 |
| 5996. | AC007115 | Genbank | 4895146 |
| 5997. | AC007172 | Genbank | 4731066 |
| 5998. | AC007192 | Genbank | 4558639 |
| 5999. | AC007198 | Genbank | 4726133 |
| 6000. | AC007199 | Genbank | 4558768 |
| 6001. | AC007207 | Genbank | 6466489 |
| 6002. | AC007254 | Genbank | 6560919 |
| 6003. | AC007271 | Genbank | 7408074 |
| 6004. | AC007281 | Genbank | 4926893 |
| 6005. | AC007283 | Genbank | 5931446 |
| 6006. | AC007319 | Genbank | 5001496 |
| 6007. | AC007347 | Genbank | 6806840 |
| 6008. | AC007406 | Genbank | 4689442 |
| 6009. | AC007421 | Genbank | 5788043 |
| 6010. | AC007428 | Genbank | 9369503 |
| 6011. | AC007435 | Genbank | 5091640 |
| 6012. | AC007528 | Genbank | 5263309 |
| 6013. | AC007541 | Genbank | 4982536 |
| 6014. | AC007546 | Genbank | 5668756 |
| 6015. | AC007563 | Genbank | 6289217 |
| 6016. | AC007671 | Genbank | 6600816 |
| 6017. | AC007678 | Genbank | 7656639 |
| 6018. | AC007707 | Genbank | 5764724 |
| 6019. | AC007739 | Genbank | 7534255 |
| 6020. | AC007793 | Genbank | 5051724 |
| 6021. | AC007845 | Genbank | 7109495 |
| 6022. | AC007899 | Genbank | 6289213 |
| 6023. | AC007956 | Genbank | 7341426 |
| 6024. | AC007969 | Genbank | 7534288 |
| 6025. | AC008009 | Genbank | 5668757 |
| 6026. | AC008040 | Genbank | 5922025 |
| 6027. | AC008069 | Genbank | 6094640 |
| 6028. | AC008119 | Genbank | 5931374 |
| 6029. | AC008122 | Genbank | 5931373 |
| 6030. | AC008134 | Genbank | 5801655 |
| 6031. | AC008164 | Genbank | 7243886 |
| 6032. | AC008417 | Genbank | 6730695 |
| 6033. | AC008474 | Genbank | 8886964 |
| 6034. | AC008772 | Genbank | 8844106 |
| 6035. | AC008860 | Genbank | 7549618 |
| 6036. | AC008865 | Genbank | 7019284 |
| 6037. | AC008976 | Genbank | 9211196 |
| 6038. | AC009000 | Genbank | 8810256 |
| 6039. | AC009007 | Genbank | 7527772 |
| 6040. | AC009060 | Genbank | 9690317 |
| 6041. | AC009073 | Genbank | 9211198 |
| 6042. | AC009079 | Genbank | 7340296 |
| 6043. | AC009145 | Genbank | 7636352 |
| 6044. | AC009229 | Genbank | 10716650 |
| 6045. | AC009235 | Genbank | 9454633 |
| 6046. | AC009239 | Genbank | 6094637 |
| 6047. | AC009247 | Genbank | 6539155 |
| 6048. | AC009311 | Genbank | 10716649 |
| 6049. | AC009314 | Genbank | 8748928 |
| 6050. | AC009319 | Genbank | 9558561 |
| 6051. | AC009336 | Genbank | 10835353 |
| 6052. | AC009475 | Genbank | 8468974 |
| 6053. | AC009478 | Genbank | 7770675 |
| 6054. | AC009509 | Genbank | 6492473 |
| 6055. | AC009516 | Genbank | 6437562 |
| 6056. | AC009958 | Genbank | 7408124 |
| 6057. | AC010092 | Genbank | 8099095 |
| 6058. | AC010102 | Genbank | 7670242 |
| 6059. | AC010168 | Genbank | 6855156 |
| 6060. | AC010183 | Genbank | 6143843 |
| 6061. | AC010202 | Genbank | 6598666 |
| 6062. | AC010282 | Genbank | 9958012 |
| 6063. | AC010352 | Genbank | 7109394 |
| 6064. | AC010436 | Genbank | 6938861 |
| 6065. | AC010457 | Genbank | 8122195 |
| 6066. | AC010491 | Genbank | 7651783 |
| 6067. | AC010605 | Genbank | 7019286 |
| 6068. | AC010644 | Genbank | 8567762 |
| 6069. | AC010876 | Genbank | 7243901 |
| 6070. | AC011088 | Genbank | 7960348 |
| 6071. | AC011362 | Genbank | 6094545 |
| 6072. | AC011452 | Genbank | 9885999 |
| 6073. | AC011455 | Genbank | 7839903 |
| 6074. | AC011464 | Genbank | 8886976 |
| 6075. | AC011500 | Genbank | 9929688 |
| 6076. | AC011523 | Genbank | 8778953 |
| 6077. | AC011599 | Genbank | 7113871 |
| 6078. | AC011716 | Genbank | 9857511 |
| 6079. | AC011742 | Genbank | 9887800 |
| 6080. | AC011815 | Genbank | 9280746 |
| 6081. | AC012309 | Genbank | 8886978 |
| 6082. | AC012467 | Genbank | 7363385 |
| 6083. | AC015853 | Genbank | 6721261 |
| 6084. | AC016254 | Genbank | 9845100 |
| 6085. | AC016396 | Genbank | 7715613 |
| 6086. | AC016397 | Genbank | 8567750 |
| 6087. | AC016656 | Genbank | 8099264 |
| 6088. | AC016939 | Genbank | 7114618 |
| 6089. | AC016940 | Genbank | 7381646 |
| 6090. | AC017016 | Genbank | 8954235 |
| 6091. | AC018452 | Genbank | 8567646 |
| 6092. | AC018513 | Genbank | 9280696 |
| 6093. | AC018764 | Genbank | 11128366 |
| 6094. | AC018766 | Genbank | 7272097 |
| 6095. | AC018797 | Genbank | 8747372 |
| 6096. | AC019181 | Genbank | 8748861 |
| 6097. | AC019227 | Genbank | 9755498 |
| 6098. | AC020626 | Genbank | 7656676 |
| 6099. | AC020633 | Genbank | 7658301 |
| 6100. | AC020663 | Genbank | 6682593 |
| 6101. | AC020751 | Genbank | 9858927 |
| 6102. | AC020910 | Genbank | 9558584 |
| 6103. | AC021036 | Genbank | 9885981 |
| 6104. | AC021037 | Genbank | 9857500 |
| 6105. | AC022149 | Genbank | 7527774 |
| 6106. | AC022336 | Genbank | 7684378 |
| 6107. | AC022515 | Genbank | 8810270 |
| 6108. | AC023480 | Genbank | 10881053 |
| 6109. | AC023796 | Genbank | 9558557 |
| 6110. | AC024083 | Genbank | 9789634 |
| 6111. | AC024084 | Genbank | 9256843 |
| 6112. | AC037199 | Genbank | 7527775 |
| 6113. | AC064878 | Genbank | 8698767 |
| 6114. | AC067967 | Genbank | 7920832 |
| 6115. | AC078899 | Genbank | 9789611 |
| 6116. | AF000231 | Genbank | 2149974 |
| 6117. | AF000364 | Genbank | 2697102 |
| 6118. | AF000381 | Genbank | 2565195 |
| 6119. | AF000652 | Genbank | 2795862 |
| 6120. | AF000993 | Genbank | 2580571 |
| 6121. | AF003837 | Genbank | 2228792 |
| 6122. | AF004561 | Genbank | 2209346 |
| 6123. | AF004849 | Genbank | 2627330 |
| 6124. | AF006082 | Genbank | 2282029 |
| 6125. | AF006086 | Genbank | 2282037 |
| 6126. | AF006386 | Genbank | 2352533 |
| 6127. | AF006515 | Genbank | 2645432 |
| 6128. | AF007748 | Genbank | 2440003 |
| 6129. | AF011333 | Genbank | 3165456 |
| 6130. | AF012072 | Genbank | 9967556 |
| 6131. | AF013168 | Genbank | 2331280 |
| 6132. | AF013759 | Genbank | 3153208 |
| 6133. | AF013970 | Genbank | 2801421 |
| 6134. | AF015040 | Genbank | 4102704 |
| 6135. | AF015287 | Genbank | 4102714 |
| 6136. | AF015812 | Genbank | 2599359 |
| 6137. | AF016507 | Genbank | 2909776 |
| 6138. | AF017456 | Genbank | 2408231 |
| 6139. | AF017782 | Genbank | 3986404 |
| 6140. | AF020202 | Genbank | 2431999 |
| 6141. | AF020736 | Genbank | 3450954 |
| 6142. | AF021819 | Genbank | 2460317 |
| 6143. | AF023268 | Genbank | 2564910 |
| 6144. | AF023917 | Genbank | 3387789 |
| 6145. | AF024617 | Genbank | 7212804 |
| 6146. | AF025439 | Genbank | 2815605 |
| 6147. | AF026029 | Genbank | 2895275 |
| 6148. | AF026126 | Genbank | 2828601 |
| 6149. | AF026166 | Genbank | 4090928 |
| 6150. | AF026169 | Genbank | 5091668 |
| 6151. | AF026291 | Genbank | 2559007 |
| 6152. | AF026292 | Genbank | 2559009 |
| 6153. | AF026850 | Genbank | 3603229 |
| 6154. | AF026977 | Genbank | 2583080 |
| 6155. | AF029750 | Genbank | 2587057 |
| 6156. | AF029786 | Genbank | 3403166 |
| 6157. | AF029890 | Genbank | 2745882 |
| 6158. | AF031141 | Genbank | 2623259 |
| 6159. | AF031379 | Genbank | 4894208 |
| 6160. | AF032922 | Genbank | 3820481 |
| 6161. | AF033026 | Genbank | 3378100 |
| 6162. | AF034795 | Genbank | 2853613 |
| 6163. | AF035286 | Genbank | 2661038 |
| 6164. | AF035737 | Genbank | 2827179 |
| 6165. | AF035840 | Genbank | 3800739 |
| 6166. | AF036613 | Genbank | 2687639 |
| 6167. | AF038176 | Genbank | 2795896 |
| 6168. | AF038187 | Genbank | 2795907 |
| 6169. | AF038202 | Genbank | 2795923 |
| 6170. | AF038954 | Genbank | 3329377 |
| 6171. | AF038955 | Genbank | 3329379 |
| 6172. | AF038960 | Genbank | 3329389 |
| 6173. | AF039656 | Genbank | 2773159 |
| 6174. | AF039693 | Genbank | 3170185 |
| 6175. | AF040707 | Genbank | 3688794 |
| 6176. | AF040964 | Genbank | 2792363 |
| 6177. | AF041432 | Genbank | 2791803 |
| 6178. | AF041459 | Genbank | 2827291 |
| 6179. | AF041483 | Genbank | 3493528 |
| 6180. | AF042081 | Genbank | 3337419 |
| 6181. | AF042166 | Genbank | 3298596 |
| 6182. | AF042385 | Genbank | 2828148 |
| 6183. | AF043325 | Genbank | 3005064 |
| 6184. | AF043644 | Genbank | 7408009 |
| 6185. | AF044671 | Genbank | 4105274 |
| 6186. | AF044955 | Genbank | 4164443 |
| 6187. | AF044958 | Genbank | 4164447 |
| 6188. | AF044959 | Genbank | 3348136 |
| 6189. | AF046059 | Genbank | 4105471 |
| 6190. | AF047020 | Genbank | 4204096 |
| 6191. | AF047182 | Genbank | 2909855 |
| 6192. | AF047184 | Genbank | 2909859 |
| 6193. | AF047433 | Genbank | 3335505 |
| 6194. | AF047435 | Genbank | 3335125 |
| 6195. | AF047439 | Genbank | 3335133 |
| 6196. | AF047440 | Genbank | 3335135 |
| 6197. | AF047470 | Genbank | 2906145 |
| 6198. | AF049140 | Genbank | 2947300 |
| 6199. | AF049907 | Genbank | 2952307 |
| 6200. | AF050637 | Genbank | 4164451 |
| 6201. | AF050638 | Genbank | 5326819 |
| 6202. | AF050639 | Genbank | 4164453 |
| 6203. | AF051323 | Genbank | 4091777 |
| 6204. | AF051334 | Genbank | 3126794 |
| 6205. | AF051894 | Genbank | 3095110 |
| 6206. | AF052051 | Genbank | 5668857 |
| 6207. | AF052092 | Genbank | 3360398 |
| 6208. | AF052094 | Genbank | 3360400 |
| 6209. | AF052135 | Genbank | 3360444 |
| 6210. | AF052164 | Genbank | 3360475 |
| 6211. | AF052169 | Genbank | 3360480 |
| 6212. | AF052178 | Genbank | 3360489 |
| 6213. | AF052182 | Genbank | 3360494 |
| 6214. | AF052578 | Genbank | 2967847 |
| 6215. | AF052642 | Genbank | 2981453 |
| 6216. | AF052955 | Genbank | 8117711 |
| 6217. | AF053551 | Genbank | 3283048 |
| 6218. | AF053630 | Genbank | 2997691 |
| 6219. | AF054175 | Genbank | 3341993 |
| 6220. | AF054184 | Genbank | 4092055 |
| 6221. | AF054187 | Genbank | 4092059 |
| 6222. | AF054840 | Genbank | 2997744 |
| 6223. | AF054988 | Genbank | 3005699 |
| 6224. | AF054990 | Genbank | 3005703 |
| 6225. | AF055032 | Genbank | 3005762 |
| 6226. | AF055033 | Genbank | 3005763 |
| 6227. | AF056087 | Genbank | 3033550 |
| 6228. | AF058696 | Genbank | 3098674 |
| 6229. | AF060703 | Genbank | 6648622 |
| 6230. | AF061016 | Genbank | 3127126 |
| 6231. | AF061258 | Genbank | 3108092 |
| 6232. | AF061736 | Genbank | 4335936 |
| 6233. | AF061737 | Genbank | 4335938 |
| 6234. | AF061739 | Genbank | 4335942 |
| 6235. | AF062534 | Genbank | 3851521 |
| 6236. | AF063020 | Genbank | 3283351 |
| 6237. | AF063605 | Genbank | 4071360 |
| 6238. | AF064084 | Genbank | 3135668 |
| 6239. | AF064257 | Genbank | 5881960 |
| 6240. | AF064603 | Genbank | 3152659 |
| 6241. | AF064861 | Genbank | 3171154 |
| 6242. | AF065388 | Genbank | 3152700 |
| 6243. | AF067170 | Genbank | 4894373 |
| 6244. | AF067420 | Genbank | 3201899 |
| 6245. | AF067972 | Genbank | 4927369 |
| 6246. | AF068235 | Genbank | 4321975 |
| 6247. | AF068295 | Genbank | 7634784 |
| 6248. | AF068846 | Genbank | 3201999 |
| 6249. | AF070416 | Genbank | 6578132 |
| 6250. | AF070523 | Genbank | 3764088 |
| 6251. | AF070540 | Genbank | 3387898 |
| 6252. | AF070556 | Genbank | 3387921 |
| 6253. | AF070561 | Genbank | 3387928 |
| 6254. | AF070596 | Genbank | 3387973 |
| 6255. | AF070597 | Genbank | 3387974 |
| 6256. | AF070638 | Genbank | 3283909 |
| 6257. | AF070646 | Genbank | 3283920 |
| 6258. | AF070649 | Genbank | 3283923 |
| 6259. | AF070650 | Genbank | 4454675 |
| 6260. | AF070654 | Genbank | 4454683 |
| 6261. | AF070655 | Genbank | 4454685 |
| 6262. | AF070661 | Genbank | 4454697 |
| 6263. | AF070665 | Genbank | 4454705 |
| 6264. | AF070667 | Genbank | 4454709 |
| 6265. | AF070668 | Genbank | 4454711 |
| 6266. | AF070669 | Genbank | 4454713 |
| 6267. | AF071172 | Genbank | 5107833 |
| 6268. | AF072097 | Genbank | 5725511 |
| 6269. | AF072250 | Genbank | 3800808 |
| 6270. | AF073298 | Genbank | 3641537 |
| 6271. | AF073770 | Genbank | 5305442 |
| 6272. | AF074000 | Genbank | 5577965 |
| 6273. | AF074331 | Genbank | 5052074 |
| 6274. | AF077030 | Genbank | 4689107 |
| 6275. | AF077034 | Genbank | 4689115 |
| 6276. | AF077037 | Genbank | 4689121 |
| 6277. | AF077041 | Genbank | 4689129 |
| 6278. | AF077043 | Genbank | 4689133 |
| 6279. | AF077046 | Genbank | 4689139 |
| 6280. | AF077050 | Genbank | 4689147 |
| 6281. | AF077200 | Genbank | 4679013 |
| 6282. | AF077202 | Genbank | 4679017 |
| 6283. | AF077207 | Genbank | 4679027 |
| 6284. | AF077208 | Genbank | 4679029 |
| 6285. | AF077353 | Genbank | 8885629 |
| 6286. | AF078845 | Genbank | 5531804 |
| 6287. | AF078848 | Genbank | 5531810 |
| 6288. | AF078850 | Genbank | 5531814 |
| 6289. | AF078852 | Genbank | 5531818 |
| 6290. | AF078854 | Genbank | 5531822 |
| 6291. | AF078855 | Genbank | 5531824 |
| 6292. | AF078858 | Genbank | 5531830 |
| 6293. | AF078860 | Genbank | 5531834 |
| 6294. | AF078861 | Genbank | 5531836 |
| 6295. | AF078863 | Genbank | 5531840 |
| 6296. | AF081484 | Genbank | 3420928 |
| 6297. | AF082889 | Genbank | 4249648 |
| 6298. | AF083420 | Genbank | 5326765 |
| 6299. | AF083441 | Genbank | 5813822 |
| 6300. | AF084523 | Genbank | 3550342 |
| 6301. | AF084555 | Genbank | 5813858 |
| 6302. | AF084943 | Genbank | 4191339 |
| 6303. | AF085355 | Genbank | 5114044 |
| 6304. | AF085734 | Genbank | 4808556 |
| 6305. | AF085832 | Genbank | 3483146 |
| 6306. | AF086172 | Genbank | 3483517 |
| 6307. | AF086234 | Genbank | 3483579 |
| 6308. | AF086292 | Genbank | 3483637 |
| 6309. | AF086557 | Genbank | 3483902 |
| 6310. | AF086559 | Genbank | 3483904 |
| 6311. | AF087659 | Genbank | 4191345 |
| 6312. | AF088038 | Genbank | 3523244 |
| 6313. | AF088070 | Genbank | 3523276 |
| 6314. | AF088851 | Genbank | 9621743 |
| 6315. | AF090891 | Genbank | 6690159 |
| 6316. | AF090913 | Genbank | 6690199 |
| 6317. | AF090947 | Genbank | 6690255 |
| 6318. | AF091035 | Genbank | 6002584 |
| 6319. | AF091076 | Genbank | 3859989 |
| 6320. | AF093535 | Genbank | 5114106 |
| 6321. | AF095192 | Genbank | 4322819 |
| 6322. | AF095352 | Genbank | 5771391 |
| 6323. | AF097362 | Genbank | 6165617 |
| 6324. | AF097492 | Genbank | 6002670 |
| 6325. | AF097535 | Genbank | 6165619 |
| 6326. | AF097725 | Genbank | 4323032 |
| 6327. | AF099013 | Genbank | 5081373 |
| 6328. | AF099100 | Genbank | 8885719 |
| 6329. | AF100620 | Genbank | 4808630 |
| 6330. | AF100748 | Genbank | 5410281 |
| 6331. | AF100753 | Genbank | 5410291 |
| 6332. | AF100755 | Genbank | 5410295 |
| 6333. | AF100756 | Genbank | 5410297 |
| 6334. | AF100757 | Genbank | 5410299 |
| 6335. | AF100761 | Genbank | 5410307 |
| 6336. | AF102803 | Genbank | 4092759 |
| 6337. | AF104238 | Genbank | 5880486 |
| 6338. | AF105253 | Genbank | 7532779 |
| 6339. | AF105423 | Genbank | 4689194 |
| 6340. | AF106564 | Genbank | 3941725 |
| 6341. | AF106862 | Genbank | 5757883 |
| 6342. | AF106966 | Genbank | 4008088 |
| 6343. | AF110643 | Genbank | 5730475 |
| 6344. | AF110731 | Genbank | 6103723 |
| 6345. | AF110774 | Genbank | 6523794 |
| 6346. | AF111167 | Genbank | 4572570 |
| 6347. | AF111170 | Genbank | 5468518 |
| 6348. | AF112222 | Genbank | 6563229 |
| 6349. | AF112227 | Genbank | 4545218 |
| 6350. | AF112299 | Genbank | 7657559 |
| 6351. | AF113011 | Genbank | 6642745 |
| 6352. | AF113127 | Genbank | 6523816 |
| 6353. | AF113129 | Genbank | 6523820 |
| 6354. | AF113251 | Genbank | 5852417 |
| 6355. | AF114264 | Genbank | 4768674 |
| 6356. | AF116030 | Genbank | 4164082 |
| 6357. | AF116606 | Genbank | 7959715 |
| 6358. | AF116612 | Genbank | 7959727 |
| 6359. | AF116628 | Genbank | 7959757 |
| 6360. | AF116639 | Genbank | 7959779 |
| 6361. | AF116679 | Genbank | 7959856 |
| 6362. | AF116692 | Genbank | 7959882 |
| 6363. | AF116719 | Genbank | 7959936 |
| 6364. | AF116827 | Genbank | 4768830 |
| 6365. | AF117230 | Genbank | 6563233 |
| 6366. | AF117231 | Genbank | 6563235 |
| 6367. | AF117236 | Genbank | 6563245 |
| 6368. | AF117892 | Genbank | 5565865 |
| 6369. | AF118649 | Genbank | 6840946 |
| 6370. | AF119850 | Genbank | 7770136 |
| 6371. | AF119891 | Genbank | 7770218 |
| 6372. | AF119897 | Genbank | 7770230 |
| 6373. | AF119905 | Genbank | 7770246 |
| 6374. | AF120981 | Genbank | 5739178 |
| 6375. | AF121858 | Genbank | 4689255 |
| 6376. | AF125097 | Genbank | 5106989 |
| 6377. | AF125102 | Genbank | 5106999 |
| 6378. | AF126780 | Genbank | 6318543 |
| 6379. | AF127577 | Genbank | 7019596 |
| 6380. | AF128527 | Genbank | 4928043 |
| 6381. | AF128536 | Genbank | 5305705 |
| 6382. | AF129512 | Genbank | 6469926 |
| 6383. | AF130358 | Genbank | 7547270 |
| 6384. | AF131738 | Genbank | 4406548 |
| 6385. | AF131742 | Genbank | 4406554 |
| 6386. | AF131807 | Genbank | 4406639 |
| 6387. | AF131808 | Genbank | 4406640 |
| 6388. | AF131838 | Genbank | 4406677 |
| 6389. | AF131842 | Genbank | 4406682 |
| 6390. | AF131846 | Genbank | 4406687 |
| 6391. | AF132968 | Genbank | 4680706 |
| 6392. | AF132973 | Genbank | 4680716 |
| 6393. | AF135162 | Genbank | 7259481 |
| 6394. | AF136630 | Genbank | 7416936 |
| 6395. | AF138300 | Genbank | 5532410 |
| 6396. | AF138859 | Genbank | 7340965 |
| 6397. | AF141346 | Genbank | 5031292 |
| 6398. | AF143235 | Genbank | 6449465 |
| 6399. | AF145316 | Genbank | 4929324 |
| 6400. | AF145477 | Genbank | 6910964 |
| 6401. | AF146191 | Genbank | 5678818 |
| 6402. | AF146651 | Genbank | 5020073 |
| 6403. | AF147334 | Genbank | 4761685 |
| 6404. | AF147717 | Genbank | 4877998 |
| 6405. | AF150089 | Genbank | 5107165 |
| 6406. | AF150959 | Genbank | 5031409 |
| 6407. | AF151020 | Genbank | 7106761 |
| 6408. | AF151029 | Genbank | 7106779 |
| 6409. | AF151032 | Genbank | 7106785 |
| 6410. | AF151064 | Genbank | 7106849 |
| 6411. | AF151075 | Genbank | 7106871 |
| 6412. | AF151077 | Genbank | 7106875 |
| 6413. | AF151103 | Genbank | 5758136 |
| 6414. | AF151109 | Genbank | 6166337 |
| 6415. | AF151793 | Genbank | 6424941 |
| 6416. | AF151818 | Genbank | 4929588 |
| 6417. | AF151839 | Genbank | 4929630 |
| 6418. | AF151840 | Genbank | 4929632 |
| 6419. | AF151841 | Genbank | 4929634 |
| 6420. | AF151872 | Genbank | 4929696 |
| 6421. | AF151874 | Genbank | 4929700 |
| 6422. | AF151875 | Genbank | 4929702 |
| 6423. | AF151876 | Genbank | 4929704 |
| 6424. | AF151878 | Genbank | 4929708 |
| 6425. | AF151883 | Genbank | 4929718 |
| 6426. | AF151887 | Genbank | 4929726 |
| 6427. | AF151893 | Genbank | 4929738 |
| 6428. | AF151907 | Genbank | 4929766 |
| 6429. | AF152363 | Genbank | 5669134 |
| 6430. | AF152462 | Genbank | 5565976 |
| 6431. | AF154415 | Genbank | 6090855 |
| 6432. | AF155238 | Genbank | 9186822 |
| 6433. | AF155652 | Genbank | 7677057 |
| 6434. | AF156965 | Genbank | 5731112 |
| 6435. | AF157321 | Genbank | 7688692 |
| 6436. | AF157324 | Genbank | 7688698 |
| 6437. | AF159056 | Genbank | 5566238 |
| 6438. | AF159548 | Genbank | 6525070 |
| 6439. | AF160213 | Genbank | 7677067 |
| 6440. | AF161390 | Genbank | 6841193 |
| 6441. | AF161406 | Genbank | 6841225 |
| 6442. | AF161448 | Genbank | 6841309 |
| 6443. | AF161473 | Genbank | 6841469 |
| 6444. | AF161476 | Genbank | 6841475 |
| 6445. | AF161477 | Genbank | 6841477 |
| 6446. | AF161503 | Genbank | 6841529 |
| 6447. | AF161511 | Genbank | 6841545 |
| 6448. | AF161517 | Genbank | 6841557 |
| 6449. | AF161518 | Genbank | 6841559 |
| 6450. | AF161530 | Genbank | 6841583 |
| 6451. | AF161556 | Genbank | 6841379 |
| 6452. | AF161886 | Genbank | 8886434 |
| 6453. | AF164795 | Genbank | 8895092 |
| 6454. | AF167438 | Genbank | 9622123 |
| 6455. | AF168716 | Genbank | 9437346 |
| 6456. | AF169035 | Genbank | 6466009 |
| 6457. | AF170583 | Genbank | 6318677 |
| 6458. | AF172066 | Genbank | 5733811 |
| 6459. | AF174496 | Genbank | 7108914 |
| 6460. | AF175265 | Genbank | 9622849 |
| 6461. | AF176646 | Genbank | 7542376 |
| 6462. | AF176698 | Genbank | 6467889 |
| 6463. | AF177758 | Genbank | 5853112 |
| 6464. | AF178581 | Genbank | 10800410 |
| 6465. | AF182076 | Genbank | 7453544 |
| 6466. | AF182289 | Genbank | 5919146 |
| 6467. | AF182292 | Genbank | 5919152 |
| 6468. | AF182844 | Genbank | 6003571 |
| 6469. | AF183419 | Genbank | 9963776 |
| 6470. | AF187554 | Genbank | 6653225 |
| 6471. | AF188611 | Genbank | 6470149 |
| 6472. | AF189723 | Genbank | 6826913 |
| 6473. | AF191649 | Genbank | 7739463 |
| 6474. | AF191771 | Genbank | 6601420 |
| 6475. | AF192793 | Genbank | 6601432 |
| 6476. | AF196779 | Genbank | 6180170 |
| 6477. | AF200465 | Genbank | 6690789 |
| 6478. | AF201077 | Genbank | 6456748 |
| 6479. | AF201933 | Genbank | 9295169 |
| 6480. | AF201935 | Genbank | 9295173 |
| 6481. | AF201937 | Genbank | 9295177 |
| 6482. | AF201950 | Genbank | 6563297 |
| 6483. | AF201951 | Genbank | 6563299 |
| 6484. | AF202445 | Genbank | 9081980 |
| 6485. | AF203815 | Genbank | 6979641 |
| 6486. | AF208844 | Genbank | 7582275 |
| 6487. | AF208849 | Genbank | 7582285 |
| 6488. | AF208850 | Genbank | 7582287 |
| 6489. | AF210052 | Genbank | 6606549 |
| 6490. | AF211972 | Genbank | 9885302 |
| 6491. | AF216751 | Genbank | 7341108 |
| 6492. | AF217511 | Genbank | 7688964 |
| 6493. | AF220187 | Genbank | 7689024 |
| 6494. | AF221595 | Genbank | 9651705 |
| 6495. | AF223469 | Genbank | 7578788 |
| 6496. | AF225898 | Genbank | 7021527 |
| 6497. | AF228421 | Genbank | 6942314 |
| 6498. | AF228603 | Genbank | 6984179 |
| 6499. | AF233747 | Genbank | 7576898 |
| 6500. | AF235097 | Genbank | 7417262 |
| 6501. | AF236056 | Genbank | 7271866 |
| 6502. | AF237982 | Genbank | 7533023 |
| 6503. | AF242205 | Genbank | 9837082 |
| 6504. | AF247661 | Genbank | 8101022 |
| 6505. | AF247704 | Genbank | 9963969 |
| 6506. | AF249273 | Genbank | 7582385 |
| 6507. | AF250226 | Genbank | 9049782 |
| 6508. | AF251043 | Genbank | 7542836 |
| 6509. | AF261085 | Genbank | 9802301 |
| 6510. | AF263452 | Genbank | 8778051 |
| 6511. | AF271897 | Genbank | 8896136 |
| 6512. | AF274753 | Genbank | 9502216 |
| 6513. | AF275719 | Genbank | 9082288 |
| 6514. | AF275948 | Genbank | 9247085 |
| 6515. | AF279145 | Genbank | 9857405 |
| 6516. | AF282626 | Genbank | 9082320 |
| 6517. | AF283771 | Genbank | 10281740 |
| 6518. | AF283772 | Genbank | 10281741 |
| 6519. | AF285442 | Genbank | 9230789 |
| 6520. | AF303378 | Genbank | 10186502 |
| 6521. | AJ000519 | Genbank | 2739214 |
| 6522. | AJ002572 | Genbank | 2959924 |
| 6523. | AJ004955 | Genbank | 3005935 |
| 6524. | AJ005866 | Genbank | 4008516 |
| 6525. | AJ010442 | Genbank | 3954884 |
| 6526. | AJ011915 | Genbank | 3757675 |
| 6527. | AJ011930 | Genbank | 3859769 |
| 6528. | AJ132545 | Genbank | 4753774 |
| 6529. | AJ132637 | Genbank | 5689741 |
| 6530. | AJ223324 | Genbank | 3392916 |
| 6531. | AJ223812 | Genbank | 2894518 |
| 6532. | AJ224878 | Genbank | 3402215 |
| 6533. | AJ225782 | Genbank | 4165269 |
| 6534. | AJ245874 | Genbank | 9715826 |
| 6535. | AJ249731 | Genbank | 5921469 |
| 6536. | AJ251595 | Genbank | 6491738 |
| 6537. | AJ272050 | Genbank | 10046713 |
| 6538. | AJ276485 | Genbank | 7320864 |
| 6539. | AJ276674 | Genbank | 7329720 |
| 6540. | AJ278463 | Genbank | 8671170 |
| 6541. | AJ297587 | Genbank | 10185081 |
| 6542. | AK000009 | Genbank | 7019813 |
| 6543. | AK000020 | Genbank | 7019831 |
| 6544. | AK000046 | Genbank | 7019874 |
| 6545. | AK000049 | Genbank | 7019880 |
| 6546. | AK000051 | Genbank | 7019883 |
| 6547. | AK000067 | Genbank | 7019912 |
| 6548. | AK000086 | Genbank | 7019944 |
| 6549. | AK000087 | Genbank | 7019946 |
| 6550. | AK000160 | Genbank | 7020066 |
| 6551. | AK000174 | Genbank | 7020088 |
| 6552. | AK000348 | Genbank | 7020373 |
| 6553. | AK000360 | Genbank | 7020393 |
| 6554. | AK000370 | Genbank | 7020411 |
| 6555. | AK000405 | Genbank | 7020471 |
| 6556. | AK000426 | Genbank | 7020505 |
| 6557. | AK000486 | Genbank | 7020607 |
| 6558. | AK000487 | Genbank | 7020609 |
| 6559. | AK000491 | Genbank | 7020616 |
| 6560. | AK000500 | Genbank | 7020630 |
| 6561. | AK000505 | Genbank | 7020640 |
| 6562. | AK000532 | Genbank | 7020691 |
| 6563. | AK000548 | Genbank | 7020717 |
| 6564. | AK000558 | Genbank | 7020734 |
| 6565. | AK000592 | Genbank | 7020792 |
| 6566. | AK000604 | Genbank | 7020812 |
| 6567. | AK000617 | Genbank | 7020830 |
| 6568. | AK000669 | Genbank | 7020909 |
| 6569. | AK000674 | Genbank | 7020916 |
| 6570. | AK000679 | Genbank | 7020923 |
| 6571. | AK000681 | Genbank | 7020926 |
| 6572. | AK000713 | Genbank | 7020973 |
| 6573. | AK000715 | Genbank | 7020977 |
| 6574. | AK000737 | Genbank | 7021010 |
| 6575. | AK000796 | Genbank | 7021099 |
| 6576. | AK000817 | Genbank | 7021127 |
| 6577. | AK000866 | Genbank | 7021190 |
| 6578. | AK000900 | Genbank | 7021855 |
| 6579. | AK000928 | Genbank | 7021901 |
| 6580. | AK000981 | Genbank | 7021979 |
| 6581. | AK000987 | Genbank | 7021987 |
| 6582. | AK001042 | Genbank | 7022067 |
| 6583. | AK001050 | Genbank | 7022077 |
| 6584. | AK001061 | Genbank | 7022095 |
| 6585. | AK001088 | Genbank | 7022136 |
| 6586. | AK001105 | Genbank | 7022160 |
| 6587. | AK001207 | Genbank | 7022315 |
| 6588. | AK001235 | Genbank | 7022362 |
| 6589. | AK001282 | Genbank | 7022438 |
| 6590. | AK001285 | Genbank | 7022444 |
| 6591. | AK001286 | Genbank | 7022445 |
| 6592. | AK001301 | Genbank | 7022470 |
| 6593. | AK001305 | Genbank | 7022477 |
| 6594. | AK001313 | Genbank | 7022490 |
| 6595. | AK001332 | Genbank | 7022524 |
| 6596. | AK001437 | Genbank | 7022693 |
| 6597. | AK001478 | Genbank | 7022760 |
| 6598. | AK001480 | Genbank | 7022762 |
| 6599. | AK001531 | Genbank | 7022843 |
| 6600. | AK001536 | Genbank | 7022851 |
| 6601. | AK001580 | Genbank | 7022920 |
| 6602. | AK001586 | Genbank | 7022930 |
| 6603. | AK001656 | Genbank | 7023047 |
| 6604. | AK001680 | Genbank | 7023088 |
| 6605. | AK001685 | Genbank | 7023097 |
| 6606. | AK001690 | Genbank | 7023105 |
| 6607. | AK001698 | Genbank | 7023119 |
| 6608. | AK001714 | Genbank | 7023146 |
| 6609. | AK001718 | Genbank | 7023153 |
| 6610. | AK001810 | Genbank | 7023313 |
| 6611. | AK001851 | Genbank | 7023374 |
| 6612. | AK001872 | Genbank | 7023409 |
| 6613. | AK001875 | Genbank | 7023414 |
| 6614. | AK001878 | Genbank | 7023418 |
| 6615. | AK001888 | Genbank | 7023435 |
| 6616. | AK001902 | Genbank | 7023455 |
| 6617. | AK001967 | Genbank | 7023560 |
| 6618. | AK001982 | Genbank | 7023588 |
| 6619. | AK001993 | Genbank | 7023607 |
| 6620. | AK002002 | Genbank | 7023620 |
| 6621. | AK002058 | Genbank | 7023709 |
| 6622. | AK002062 | Genbank | 7023716 |
| 6623. | AK002164 | Genbank | 7023875 |
| 6624. | AK002168 | Genbank | 7023882 |
| 6625. | AK023288 | Genbank | 10435159 |
| 6626. | AK024618 | Genbank | 10436934 |
| 6627. | AL008583 | Genbank | 4160195 |
| 6628. | AL008639 | Genbank | 6599068 |
| 6629. | AL008729 | Genbank | 2827472 |
| 6630. | AL020990 | Genbank | 3980348 |
| 6631. | AL020993 | Genbank | 3980107 |
| 6632. | AL021069 | Genbank | 2853183 |
| 6633. | AL021154 | Genbank | 3219576 |
| 6634. | AL021407 | Genbank | 2982498 |
| 6635. | AL021451 | Genbank | 2815069 |
| 6636. | AL021917 | Genbank | 10190813 |
| 6637. | AL021918 | Genbank | 3135967 |
| 6638. | AL021920 | Genbank | 3264535 |
| 6639. | AL022097 | Genbank | 3169107 |
| 6640. | AL022150 | Genbank | 3150085 |
| 6641. | AL022165 | Genbank | 3281985 |
| 6642. | AL022315 | Genbank | 3820991 |
| 6643. | AL022316 | Genbank | 4691242 |
| 6644. | AL022334 | Genbank | 3947839 |
| 6645. | AL022345 | Genbank | 6562625 |
| 6646. | AL022394 | Genbank | 9581758 |
| 6647. | AL022398 | Genbank | 3355547 |
| 6648. | AL022577 | Genbank | 3550044 |
| 6649. | AL023284 | Genbank | 3355875 |
| 6650. | AL023582 | Genbank | 3805923 |
| 6651. | AL031003 | Genbank | 4007185 |
| 6652. | AL031115 | Genbank | 4455584 |
| 6653. | AL031132 | Genbank | 9368411 |
| 6654. | AL031259 | Genbank | 3790132 |
| 6655. | AL031277 | Genbank | 4375907 |
| 6656. | AL031281 | Genbank | 4826460 |
| 6657. | AL031295 | Genbank | 4376011 |
| 6658. | AL031390 | Genbank | 5002609 |
| 6659. | AL031428 | Genbank | 5931719 |
| 6660. | AL031431 | Genbank | 4938290 |
| 6661. | AL031577 | Genbank | 3786166 |
| 6662. | AL031589 | Genbank | 4914506 |
| 6663. | AL031591 | Genbank | 6006484 |
| 6664. | AL031651 | Genbank | 6065866 |
| 6665. | AL031656 | Genbank | 5360983 |
| 6666. | AL031662 | Genbank | 9716901 |
| 6667. | AL031668 | Genbank | 10198603 |
| 6668. | AL031670 | Genbank | 4469083 |
| 6669. | AL031682 | Genbank | 9795187 |
| 6670. | AL031737 | Genbank | 4464258 |
| 6671. | AL031777 | Genbank | 10198609 |
| 6672. | AL031847 | Genbank | 9369286 |
| 6673. | AL033377 | Genbank | 4826462 |
| 6674. | AL033397 | Genbank | 4902626 |
| 6675. | AL033531 | Genbank | 6807582 |
| 6676. | AL034377 | Genbank | 4376000 |
| 6677. | AL034379 | Genbank | 5918013 |
| 6678. | AL034549 | Genbank | 9795176 |
| 6679. | AL035086 | Genbank | 4741478 |
| 6680. | AL035089 | Genbank | 6742111 |
| 6681. | AL035304 | Genbank | 4200231 |
| 6682. | AL035411 | Genbank | 6136940 |
| 6683. | AL035413 | Genbank | 6010110 |
| 6684. | AL035416 | Genbank | 4775629 |
| 6685. | AL035422 | Genbank | 5263001 |
| 6686. | AL035423 | Genbank | 4467309 |
| 6687. | AL035455 | Genbank | 9581749 |
| 6688. | AL035456 | Genbank | 9801541 |
| 6689. | AL035458 | Genbank | 6624641 |
| 6690. | AL035462 | Genbank | 10198627 |
| 6691. | AL035541 | Genbank | 10198628 |
| 6692. | AL035587 | Genbank | 6002306 |
| 6693. | AL035661 | Genbank | 6015535 |
| 6694. | AL035666 | Genbank | 4914532 |
| 6695. | AL035683 | Genbank | 7288039 |
| 6696. | AL049257 | Genbank | 4500001 |
| 6697. | AL049381 | Genbank | 4500168 |
| 6698. | AL049404 | Genbank | 4500192 |
| 6699. | AL049447 | Genbank | 4500230 |
| 6700. | AL049450 | Genbank | 4500236 |
| 6701. | AL049455 | Genbank | 4500244 |
| 6702. | AL049471 | Genbank | 4500266 |
| 6703. | AL049544 | Genbank | 4757047 |
| 6704. | AL049612 | Genbank | 5832952 |
| 6705. | AL049699 | Genbank | 5419832 |
| 6706. | AL049715 | Genbank | 9581791 |
| 6707. | AL049767 | Genbank | 10198636 |
| 6708. | AL049796 | Genbank | 8218111 |
| 6709. | AL049820 | Genbank | 8247261 |
| 6710. | AL049829 | Genbank | 8217859 |
| 6711. | AL049835 | Genbank | 5708093 |
| 6712. | AL049843 | Genbank | 5830432 |
| 6713. | AL049845 | Genbank | 5777574 |
| 6714. | AL049868 | Genbank | 9663379 |
| 6715. | AL049873 | Genbank | 8176896 |
| 6716. | AL049932 | Genbank | 4884176 |
| 6717. | AL049949 | Genbank | 4884196 |
| 6718. | AL049957 | Genbank | 4884209 |
| 6719. | AL050037 | Genbank | 4884277 |
| 6720. | AL050161 | Genbank | 4884375 |
| 6721. | AL050197 | Genbank | 4884434 |
| 6722. | AL050203 | Genbank | 4884442 |
| 6723. | AL050228 | Genbank | 4884472 |
| 6724. | AL050269 | Genbank | 4886444 |
| 6725. | AL050277 | Genbank | 4886454 |
| 6726. | AL050331 | Genbank | 5668655 |
| 6727. | AL050363 | Genbank | 4914597 |
| 6728. | AL050380 | Genbank | 4914582 |
| 6729. | AL078477 | Genbank | 5419778 |
| 6730. | AL078596 | Genbank | 6010168 |
| 6731. | AL078612 | Genbank | 5419788 |
| 6732. | AL079295 | Genbank | 5102607 |
| 6733. | AL079341 | Genbank | 8655533 |
| 6734. | AL080089 | Genbank | 5262506 |
| 6735. | AL080111 | Genbank | 5262538 |
| 6736. | AL080135 | Genbank | 5262576 |
| 6737. | AL080192 | Genbank | 5262673 |
| 6738. | AL080206 | Genbank | 5262692 |
| 6739. | AL080210 | Genbank | 5262699 |
| 6740. | AL096719 | Genbank | 5419854 |
| 6741. | AL096771 | Genbank | 6165369 |
| 6742. | AL096791 | Genbank | 6066332 |
| 6743. | AL096800 | Genbank | 7242638 |
| 6744. | AL096865 | Genbank | 9581792 |
| 6745. | AL096888 | Genbank | 9581828 |
| 6746. | AL109612 | Genbank | 5881342 |
| 6747. | AL109766 | Genbank | 7159614 |
| 6748. | AL109797 | Genbank | 8249456 |
| 6749. | AL109839 | Genbank | 6634462 |
| 6750. | AL109845 | Genbank | 9581793 |
| 6751. | AL109847 | Genbank | 6434631 |
| 6752. | AL109935 | Genbank | 7799423 |
| 6753. | AL109939 | Genbank | 6523730 |
| 6754. | AL110125 | Genbank | 5817019 |
| 6755. | AL110126 | Genbank | 5817020 |
| 6756. | AL110129 | Genbank | 5817024 |
| 6757. | AL110141 | Genbank | 5817036 |
| 6758. | AL110183 | Genbank | 5817095 |
| 6759. | AL110185 | Genbank | 5817098 |
| 6760. | AL110193 | Genbank | 5817109 |
| 6761. | AL110194 | Genbank | 5817111 |
| 6762. | AL110197 | Genbank | 5817115 |
| 6763. | AL110252 | Genbank | 5817211 |
| 6764. | AL117341 | Genbank | 7327972 |
| 6765. | AL117342 | Genbank | 6580447 |
| 6766. | AL117412 | Genbank | 5912102 |
| 6767. | AL117596 | Genbank | 5912164 |
| 6768. | AL117644 | Genbank | 5912234 |
| 6769. | AL117666 | Genbank | 5912264 |
| 6770. | AL117672 | Genbank | 7271126 |
| 6771. | AL117694 | Genbank | 8248721 |
| 6772. | AL121578 | Genbank | 5931935 |
| 6773. | AL121583 | Genbank | 9588417 |
| 6774. | AL121601 | Genbank | 7159760 |
| 6775. | AL121716 | Genbank | 7159797 |
| 6776. | AL121723 | Genbank | 7406637 |
| 6777. | AL121755 | Genbank | 7406634 |
| 6778. | AL121769 | Genbank | 6433831 |
| 6779. | AL121772 | Genbank | 8648920 |
| 6780. | AL121787 | Genbank | 7161753 |
| 6781. | AL121790 | Genbank | 8919824 |
| 6782. | AL121808 | Genbank | 7105860 |
| 6783. | AL121870 | Genbank | 7161190 |
| 6784. | AL121920 | Genbank | 9663353 |
| 6785. | AL121985 | Genbank | 7161187 |
| 6786. | AL121989 | Genbank | 8919105 |
| 6787. | AL122003 | Genbank | 6580480 |
| 6788. | AL122047 | Genbank | 6093240 |
| 6789. | AL122054 | Genbank | 6093250 |
| 6790. | AL122061 | Genbank | 6102852 |
| 6791. | AL122127 | Genbank | 6723485 |
| 6792. | AL132642 | Genbank | 8217878 |
| 6793. | AL132656 | Genbank | 8439394 |
| 6794. | AL132716 | Genbank | 8217880 |
| 6795. | AL132987 | Genbank | 8217887 |
| 6796. | AL133096 | Genbank | 6453550 |
| 6797. | AL133117 | Genbank | 6453608 |
| 6798. | AL133228 | Genbank | 8217426 |
| 6799. | AL133230 | Genbank | 8574103 |
| 6800. | AL133241 | Genbank | 7630038 |
| 6801. | AL133283 | Genbank | 7267032 |
| 6802. | AL133299 | Genbank | 7708221 |
| 6803. | AL133330 | Genbank | 9650514 |
| 6804. | AL133367 | Genbank | 7708222 |
| 6805. | AL133438 | Genbank | 6562637 |
| 6806. | AL133502 | Genbank | 8217902 |
| 6807. | AL133551 | Genbank | 9407715 |
| 6808. | AL133553 | Genbank | 9187146 |
| 6809. | AL133577 | Genbank | 6599158 |
| 6810. | AL133580 | Genbank | 6599163 |
| 6811. | AL133602 | Genbank | 6599209 |
| 6812. | AL133611 | Genbank | 6599222 |
| 6813. | AL133628 | Genbank | 6599269 |
| 6814. | AL135744 | Genbank | 6682291 |
| 6815. | AL135786 | Genbank | 8894169 |
| 6816. | AL135787 | Genbank | 9588110 |
| 6817. | AL135923 | Genbank | 7799631 |
| 6818. | AL135978 | Genbank | 7452884 |
| 6819. | AL136109 | Genbank | 9501152 |
| 6820. | AL136139 | Genbank | 8217463 |
| 6821. | AL136173 | Genbank | 8217465 |
| 6822. | AL136179 | Genbank | 8649149 |
| 6823. | AL136419 | Genbank | 7329702 |
| 6824. | AL136531 | Genbank | 11139883 |
| 6825. | AL136985 | Genbank | 9542710 |
| 6826. | AL137013 | Genbank | 8217489 |
| 6827. | AL137067 | Genbank | 8452467 |
| 6828. | AL137221 | Genbank | 9650522 |
| 6829. | AL137226 | Genbank | 7708225 |
| 6830. | AL137263 | Genbank | 6807692 |
| 6831. | AL137294 | Genbank | 6807754 |
| 6832. | AL137412 | Genbank | 6807964 |
| 6833. | AL137440 | Genbank | 6808001 |
| 6834. | AL137489 | Genbank | 6808110 |
| 6835. | AL137529 | Genbank | 6808199 |
| 6836. | AL137543 | Genbank | 6808222 |
| 6837. | AL137571 | Genbank | 6808280 |
| 6838. | AL137638 | Genbank | 6808439 |
| 6839. | AL137650 | Genbank | 6807715 |
| 6840. | AL137663 | Genbank | 6807784 |
| 6841. | AL137784 | Genbank | 9863487 |
| 6842. | AL137800 | Genbank | 9926422 |
| 6843. | AL137861 | Genbank | 9187172 |
| 6844. | AL138752 | Genbank | 8452480 |
| 6845. | AL138758 | Genbank | 8670587 |
| 6846. | AL138761 | Genbank | 8573811 |
| 6847. | AL139316 | Genbank | 8217940 |
| 6848. | AL139317 | Genbank | 8217941 |
| 6849. | AL139803 | Genbank | 8670911 |
| 6850. | AL157444 | Genbank | 7018445 |
| 6851. | AL157481 | Genbank | 7018522 |
| 6852. | AL158206 | Genbank | 8977646 |
| 6853. | AL158801 | Genbank | 8574301 |
| 6854. | AL159140 | Genbank | 7406476 |
| 6855. | AL161665 | Genbank | 8546785 |
| 6856. | AL161725 | Genbank | 10045359 |
| 6857. | AL161952 | Genbank | 7328002 |
| 6858. | AL161967 | Genbank | 7328055 |
| 6859. | AL162049 | Genbank | 7328093 |
| 6860. | AL162151 | Genbank | 8655500 |
| 6861. | AL163202 | Genbank | 7717242 |
| 6862. | AL163249 | Genbank | 7717307 |
| 6863. | AL163285 | Genbank | 7717384 |
| 6864. | AL353759 | Genbank | 8745068 |
| 6865. | AL353771 | Genbank | 8894296 |
| 6866. | AL353950 | Genbank | 7669991 |
| 6867. | AL354720 | Genbank | 8979470 |
| 6868. | AL354829 | Genbank | 8670914 |
| 6869. | AL355112 | Genbank | 7708237 |
| 6870. | AL355520 | Genbank | 9863726 |
| 6871. | AL355708 | Genbank | 7799100 |
| 6872. | AL357196 | Genbank | 8247633 |
| 6873. | AL359052 | Genbank | 8518175 |
| 6874. | AL359332 | Genbank | 8573959 |
| 6875. | AL359397 | Genbank | 8574316 |
| 6876. | AL359591 | Genbank | 8655656 |
| 6877. | AL390090 | Genbank | 9368539 |
| 6878. | AL390139 | Genbank | 9368836 |
| 6879. | AL442077 | Genbank | 10241715 |
| 6880. | AP000009 | Genbank | 4666255 |
| 6881. | AP000020 | Genbank | 4666264 |
| 6882. | AP000044 | Genbank | 3132354 |
| 6883. | AP000054 | Genbank | 3132364 |
| 6884. | AP000057 | Genbank | 3133144 |
| 6885. | AP000348 | Genbank | 5103011 |
| 6886. | AP000356 | Genbank | 5103019 |
| 6887. | AP000426 | Genbank | 8698836 |
| 6888. | AP000497 | Genbank | 5926684 |
| 6889. | AP000523 | Genbank | 5931501 |
| 6890. | AP000554 | Genbank | 5931540 |
| 6891. | AP000555 | Genbank | 5931541 |
| 6892. | AP000693 | Genbank | 6693637 |
| 6893. | AP000953 | Genbank | 6693632 |
| 6894. | AP000959 | Genbank | 6863077 |
| 6895. | AP000962 | Genbank | 6942330 |
| 6896. | AP001053 | Genbank | 6693603 |
| 6897. | AP001136 | Genbank | 6970360 |
| 6898. | AP001137 | Genbank | 6970361 |
| 6899. | AP001340 | Genbank | 7209828 |
| 6900. | AP001346 | Genbank | 7209834 |
| 6901. | AP001615 | Genbank | 7670569 |
| 6902. | AP001625 | Genbank | 7670579 |
| 6903. | AP002022 | Genbank | 7798582 |
| 6904. | AP002027 | Genbank | 9293862 |
| 6905. | AP002028 | Genbank | 9293863 |
| 6906. | AX011717 | Genbank | 9998241 |
| 6907. | AX013067 | Genbank | 10040233 |
| 6908. | AX013082 | Genbank | 10040248 |
| 6909. | AX013102 | Genbank | 10040268 |
| 6910. | AX013777 | Genbank | 10040487 |
| 6911. | AX014108 | Genbank | 10040578 |
| 6912. | AX014111 | Genbank | 10040581 |
| 6913. | AX014157 | Genbank | 10040604 |
| 6914. | AX014301 | Genbank | 10040655 |
| 6915. | AX014351 | Genbank | 10040705 |
| 6916. | AX014825 | Genbank | 10041092 |
| 6917. | AX014868 | Genbank | 10041135 |
| 6918. | AX014888 | Genbank | 10041155 |
| 6919. | AX014891 | Genbank | 10041158 |
| 6920. | AX014908 | Genbank | 10041175 |
| 6921. | AX014915 | Genbank | 10041182 |
| 6922. | AX015387 | Genbank | 10041367 |
| 6923. | AX015394 | Genbank | 10041374 |
| 6924. | AX015398 | Genbank | 10041378 |
| 6925. | AX015400 | Genbank | 10041380 |
| 6926. | AX015525 | Genbank | 10041407 |
| 6927. | AX017296 | Genbank | 10042214 |
| 6928. | AX017306 | Genbank | 10042224 |
| 6929. | AX017475 | Genbank | 10042272 |
| 6930. | AX017496 | Genbank | 10042293 |
| 6931. | AX017850 | Genbank | 10042453 |
| 6932. | AX017859 | Genbank | 10042462 |
| 6933. | AY007110 | Genbank | 9956004 |
| 6934. | AY007135 | Genbank | 9956038 |
| 6935. | AY007153 | Genbank | 9956064 |
| 6936. | D00017 | Genbank | 219909 |
| 6937. | D00022 | Genbank | 219653 |
| 6938. | D00422 | Genbank | 220063 |
| 6939. | D00760 | Genbank | 220023 |
| 6940. | D00761 | Genbank | 220025 |
| 6941. | D00860 | Genbank | 220019 |
| 6942. | D01059 | Genbank | 219887 |
| 6943. | D10040 | Genbank | 219899 |
| 6944. | D12775 | Genbank | 219456 |
| 6945. | D13119 | Genbank | 285909 |
| 6946. | D13639 | Genbank | 285990 |
| 6947. | D13757 | Genbank | 219458 |
| 6948. | D13866 | Genbank | 433410 |
| 6949. | D14043 | Genbank | 219924 |
| 6950. | D14524 | Genbank | 285902 |
| 6951. | D14530 | Genbank | 414348 |
| 6952. | D14658 | Genbank | 285940 |
| 6953. | D14659 | Genbank | 285942 |
| 6954. | D14661 | Genbank | 285946 |
| 6955. | D14662 | Genbank | 285948 |
| 6956. | D14694 | Genbank | 603801 |
| 6957. | D14696 | Genbank | 285962 |
| 6958. | D15057 | Genbank | 493244 |
| 6959. | D16111 | Genbank | 435637 |
| 6960. | D16234 | Genbank | 303617 |
| 6961. | D16469 | Genbank | 758583 |
| 6962. | D16562 | Genbank | 506336 |
| 6963. | D16563 | Genbank | 468447 |
| 6964. | D16937 | Genbank | 598856 |
| 6965. | D17400 | Genbank | 451207 |
| 6966. | D21090 | Genbank | 498147 |
| 6967. | D21254 | Genbank | 575577 |
| 6968. | D21260 | Genbank | 434760 |
| 6969. | D21262 | Genbank | 434764 |
| 6970. | D23662 | Genbank | 432362 |
| 6971. | D25542 | Genbank | 662389 |
| 6972. | D26308 | Genbank | 1384067 |
| 6973. | D26598 | Genbank | 565646 |
| 6974. | D28480 | Genbank | 516759 |
| 6975. | D31764 | Genbank | 498153 |
| 6976. | D31767 | Genbank | 505091 |
| 6977. | D31885 | Genbank | 505097 |
| 6978. | D38047 | Genbank | 1037163 |
| 6979. | D38048 | Genbank | 1531532 |
| 6980. | D38551 | Genbank | 1531549 |
| 6981. | D38583 | Genbank | 560790 |
| 6982. | D44467 | Genbank | 976226 |
| 6983. | D45131 | Genbank | 1304103 |
| 6984. | D45248 | Genbank | 1008914 |
| 6985. | D45887 | Genbank | 665587 |
| 6986. | D49355 | Genbank | 1020405 |
| 6987. | D49396 | Genbank | 682747 |
| 6988. | D49400 | Genbank | 1395161 |
| 6989. | D49489 | Genbank | 1136742 |
| 6990. | D49490 | Genbank | 1072306 |
| 6991. | D49737 | Genbank | 2588778 |
| 6992. | D49914 | Genbank | 7768885 |
| 6993. | D50063 | Genbank | 971269 |
| 6994. | D50372 | Genbank | 2605593 |
| 6995. | D50374 | Genbank | 2605597 |
| 6996. | D50405 | Genbank | 1665722 |
| 6997. | D50420 | Genbank | 2618577 |
| 6998. | D50525 | Genbank | 1167502 |
| 6999. | D50663 | Genbank | 1747307 |
| 7000. | D55654 | Genbank | 1255603 |
| 7001. | D55696 | Genbank | 1890049 |
| 7002. | D63486 | Genbank | 1469885 |
| 7003. | D63861 | Genbank | 1769811 |
| 7004. | D63874 | Genbank | 968887 |
| 7005. | D64015 | Genbank | 2281005 |
| 7006. | D67031 | Genbank | 2696053 |
| 7007. | D78014 | Genbank | 1330241 |
| 7008. | D78514 | Genbank | 4432955 |
| 7009. | D79205 | Genbank | 1754620 |
| 7010. | D79983 | Genbank | 1136383 |
| 7011. | D79996 | Genbank | 1136407 |
| 7012. | D82061 | Genbank | 1616919 |
| 7013. | D83004 | Genbank | 1181557 |
| 7014. | D83077 | Genbank | 1304131 |
| 7015. | D83780 | Genbank | 1228042 |
| 7016. | D84488 | Genbank | 2388543 |
| 7017. | D85181 | Genbank | 1906795 |
| 7018. | D85433 | Genbank | 1841371 |
| 7019. | D86198 | Genbank | 3062805 |
| 7020. | D86978 | Genbank | 1504029 |
| 7021. | D86985 | Genbank | 6634002 |
| 7022. | D87023 | Genbank | 2114292 |
| 7023. | D87127 | Genbank | 1817551 |
| 7024. | D87434 | Genbank | 1665762 |
| 7025. | D87445 | Genbank | 6634006 |
| 7026. | D87666 | Genbank | 1620016 |
| 7027. | D89077 | Genbank | 1694681 |
| 7028. | D89092 | Genbank | 2780747 |
| 7029. | D89667 | Genbank | 1731808 |
| 7030. | D90070 | Genbank | 219475 |
| 7031. | D90158 | Genbank | 219885 |
| 7032. | E00199 | Genbank | 2168495 |
| 7033. | E00359 | Genbank | 2168646 |
| 7034. | E00882 | Genbank | 2169143 |
| 7035. | E00985 | Genbank | 2169246 |
| 7036. | E01094 | Genbank | 2169353 |
| 7037. | E01349 | Genbank | 2169606 |
| 7038. | E01816 | Genbank | 2170068 |
| 7039. | E01915 | Genbank | 2170164 |
| 7040. | E01954 | Genbank | 2170202 |
| 7041. | E02628 | Genbank | 2170856 |
| 7042. | E02651 | Genbank | 2170879 |
| 7043. | E03569 | Genbank | 2171785 |
| 7044. | E05692 | Genbank | 2173879 |
| 7045. | E06721 | Genbank | 2174903 |
| 7046. | E07218 | Genbank | 2175359 |
| 7047. | E07219 | Genbank | 2175360 |
| 7048. | E08514 | Genbank | 2176629 |
| 7049. | E08515 | Genbank | 2176630 |
| 7050. | J00152 | Genbank | 183317 |
| 7051. | J00194 | Genbank | 188231 |
| 7052. | J00196 | Genbank | 188242 |
| 7053. | J00200 | Genbank | 188411 |
| 7054. | J00204 | Genbank | 188427 |
| 7055. | J00220 | Genbank | 184743 |
| 7056. | J00230 | Genbank | 184750 |
| 7057. | J00231 | Genbank | 185041 |
| 7058. | J00241 | Genbank | 185938 |
| 7059. | J00312 | Genbank | 339963 |
| 7060. | J02611 | Genbank | 178840 |
| 7061. | J02683 | Genbank | 179246 |
| 7062. | J02853 | Genbank | 598146 |
| 7063. | J02874 | Genbank | 178346 |
| 7064. | J02876 | Genbank | 182413 |
| 7065. | J02959 | Genbank | 187174 |
| 7066. | J03040 | Genbank | 338312 |
| 7067. | J03191 | Genbank | 190385 |
| 7068. | J03507 | Genbank | 179715 |
| 7069. | J03528 | Genbank | 188671 |
| 7070. | J03592 | Genbank | 339722 |
| 7071. | J03746 | Genbank | 183655 |
| 7072. | J03779 | Genbank | 179833 |
| 7073. | J04137 | Genbank | 177782 |
| 7074. | J04164 | Genbank | 177801 |
| 7075. | J04183 | Genbank | 186929 |
| 7076. | J04456 | Genbank | 187109 |
| 7077. | J04543 | Genbank | 338243 |
| 7078. | J04621 | Genbank | 184428 |
| 7079. | J05192 | Genbank | 178026 |
| 7080. | K00409 | Genbank | 188523 |
| 7081. | K01144 | Genbank | 188469 |
| 7082. | K01615 | Genbank | 188525 |
| 7083. | K01911 | Genbank | 189273 |
| 7084. | K02569 | Genbank | 182441 |
| 7085. | K02778 | Genbank | 338922 |
| 7086. | L03558 | Genbank | 291926 |
| 7087. | L05091 | Genbank | 388030 |
| 7088. | L05092 | Genbank | 388031 |
| 7089. | L05093 | Genbank | 401844 |
| 7090. | L05186 | Genbank | 182394 |
| 7091. | L07395 | Genbank | 190218 |
| 7092. | L07648 | Genbank | 506626 |
| 7093. | L07916 | Genbank | 183067 |
| 7094. | L09159 | Genbank | 307374 |
| 7095. | L09209 | Genbank | 291855 |
| 7096. | L10400 | Genbank | 187509 |
| 7097. | L10678 | Genbank | 190387 |
| 7098. | L10819 | Genbank | 179041 |
| 7099. | L11284 | Genbank | 307183 |
| 7100. | L11667 | Genbank | 348909 |
| 7101. | L12136 | Genbank | 181536 |
| 7102. | L12168 | Genbank | 178083 |
| 7103. | L12535 | Genbank | 434050 |
| 7104. | L12711 | Genbank | 388890 |
| 7105. | L13278 | Genbank | 292414 |
| 7106. | L13850 | Genbank | 292165 |
| 7107. | L19185 | Genbank | 440307 |
| 7108. | L19739 | Genbank | 431318 |
| 7109. | L19779 | Genbank | 306828 |
| 7110. | L20688 | Genbank | 404044 |
| 7111. | L20941 | Genbank | 507251 |
| 7112. | L20967 | Genbank | 347123 |
| 7113. | L21934 | Genbank | 4878021 |
| 7114. | L25080 | Genbank | 407696 |
| 7115. | L25081 | Genbank | 407698 |
| 7116. | L28010 | Genbank | 452047 |
| 7117. | L28809 | Genbank | 454151 |
| 7118. | L29008 | Genbank | 496077 |
| 7119. | L29156 | Genbank | 465171 |
| 7120. | L34587 | Genbank | 551605 |
| 7121. | L36588 | Genbank | 598240 |
| 7122. | L36645 | Genbank | 551613 |
| 7123. | L37127 | Genbank | 4164098 |
| 7124. | L38941 | Genbank | 1008855 |
| 7125. | L40410 | Genbank | 703109 |
| 7126. | L41351 | Genbank | 862304 |
| 7127. | L42345 | Genbank | 1160933 |
| 7128. | L42856 | Genbank | 992914 |
| 7129. | L43964 | Genbank | 951202 |
| 7130. | L47168 | Genbank | 976207 |
| 7131. | L48723 | Genbank | 1246239 |
| 7132. | L49345 | Genbank | 1405422 |
| 7133. | L54057 | Genbank | 1196416 |
| 7134. | L76200 | Genbank | 1196435 |
| 7135. | L77701 | Genbank | 1280205 |
| 7136. | M10036 | Genbank | 339840 |
| 7137. | M11146 | Genbank | 182504 |
| 7138. | M11147 | Genbank | 182513 |
| 7139. | M11233 | Genbank | 181179 |
| 7140. | M11353 | Genbank | 184092 |
| 7141. | M11354 | Genbank | 184090 |
| 7142. | M11560 | Genbank | 178350 |
| 7143. | M11867 | Genbank | 188229 |
| 7144. | M11886 | Genbank | 184173 |
| 7145. | M12523 | Genbank | 178343 |
| 7146. | M12623 | Genbank | 184233 |
| 7147. | M12670 | Genbank | 182482 |
| 7148. | M12759 | Genbank | 532596 |
| 7149. | M12937 | Genbank | 182506 |
| 7150. | M13560 | Genbank | 184517 |
| 7151. | M14144 | Genbank | 340218 |
| 7152. | M14219 | Genbank | 181169 |
| 7153. | M14333 | Genbank | 181171 |
| 7154. | M14539 | Genbank | 182836 |
| 7155. | M14630 | Genbank | 339690 |
| 7156. | M15796 | Genbank | 181271 |
| 7157. | M15887 | Genbank | 181960 |
| 7158. | M16117 | Genbank | 181181 |
| 7159. | M16247 | Genbank | 178044 |
| 7160. | M16447 | Genbank | 181552 |
| 7161. | M16827 | Genbank | 177963 |
| 7162. | M17563 | Genbank | 188182 |
| 7163. | M17733 | Genbank | 339688 |
| 7164. | M17885 | Genbank | 190231 |
| 7165. | M17886 | Genbank | 190233 |
| 7166. | M19713 | Genbank | 339953 |
| 7167. | M19961 | Genbank | 180940 |
| 7168. | M20429 | Genbank | 188394 |
| 7169. | M20472 | Genbank | 187054 |
| 7170. | M20506 | Genbank | 188206 |
| 7171. | M20867 | Genbank | 183059 |
| 7172. | M21575 | Genbank | 1311702 |
| 7173. | M21895 | Genbank | 189523 |
| 7174. | M21896 | Genbank | 189525 |
| 7175. | M22348 | Genbank | 190815 |
| 7176. | M22590 | Genbank | 179418 |
| 7177. | M22865 | Genbank | 181226 |
| 7178. | M22918 | Genbank | 189019 |
| 7179. | M22920 | Genbank | 189021 |
| 7180. | M23379 | Genbank | 182971 |
| 7181. | M23492 | Genbank | 187005 |
| 7182. | M23613 | Genbank | 189271 |
| 7183. | M24194 | Genbank | 187701 |
| 7184. | M24902 | Genbank | 189618 |
| 7185. | M25246 | Genbank | 340233 |
| 7186. | M25746 | Genbank | 338323 |
| 7187. | M26325 | Genbank | 186688 |
| 7188. | M26663 | Genbank | 618463 |
| 7189. | M27717 | Genbank | 179933 |
| 7190. | M28372 | Genbank | 643575 |
| 7191. | M29063 | Genbank | 337454 |
| 7192. | M29064 | Genbank | 337452 |
| 7193. | M29877 | Genbank | 178408 |
| 7194. | M30496 | Genbank | 340073 |
| 7195. | M30938 | Genbank | 186793 |
| 7196. | M32219 | Genbank | 186618 |
| 7197. | M33197 | Genbank | 182976 |
| 7198. | M34225 | Genbank | 181399 |
| 7199. | M34600 | Genbank | 180934 |
| 7200. | M34840 | Genbank | 189620 |
| 7201. | M35252 | Genbank | 180925 |
| 7202. | M36341 | Genbank | 178984 |
| 7203. | M36501 | Genbank | 177871 |
| 7204. | M37104 | Genbank | 179274 |
| 7205. | M37583 | Genbank | 184059 |
| 7206. | M37766 | Genbank | 187518 |
| 7207. | M55268 | Genbank | 177837 |
| 7208. | M58028 | Genbank | 340071 |
| 7209. | M59830 | Genbank | 188489 |
| 7210. | M59849 | Genbank | 182591 |
| 7211. | M60484 | Genbank | 190225 |
| 7212. | M60756 | Genbank | 184085 |
| 7213. | M60922 | Genbank | 793909 |
| 7214. | M63438 | Genbank | 184847 |
| 7215. | M63625 | Genbank | 340012 |
| 7216. | M64098 | Genbank | 183891 |
| 7217. | M64110 | Genbank | 179829 |
| 7218. | M64241 | Genbank | 190813 |
| 7219. | M65212 | Genbank | 180919 |
| 7220. | M65293 | Genbank | 183764 |
| 7221. | M68867 | Genbank | 181025 |
| 7222. | M74088 | Genbank | 182396 |
| 7223. | M74525 | Genbank | 184045 |
| 7224. | M74775 | Genbank | 187151 |
| 7225. | M75139 | Genbank | 186154 |
| 7226. | M75282 | Genbank | 186134 |
| 7227. | M75883 | Genbank | 432974 |
| 7228. | M76766 | Genbank | 339489 |
| 7229. | M77016 | Genbank | 339947 |
| 7230. | M81757 | Genbank | 337732 |
| 7231. | M82882 | Genbank | 180551 |
| 7232. | M83186 | Genbank | 181404 |
| 7233. | M84526 | Genbank | 178625 |
| 7234. | M85038 | Genbank | 190189 |
| 7235. | M85079 | Genbank | 339569 |
| 7236. | M86400 | Genbank | 189952 |
| 7237. | M86737 | Genbank | 184241 |
| 7238. | M87789 | Genbank | 185361 |
| 7239. | M87790 | Genbank | 185363 |
| 7240. | M88108 | Genbank | 189499 |
| 7241. | M90309 | Genbank | 182643 |
| 7242. | M90656 | Genbank | 183038 |
| 7243. | M90746 | Genbank | 182472 |
| 7244. | M93651 | Genbank | 338038 |
| 7245. | M94556 | Genbank | 188855 |
| 7246. | M94856 | Genbank | 182353 |
| 7247. | M95571 | Genbank | 181904 |
| 7248. | M96995 | Genbank | 181975 |
| 7249. | M97922 | Genbank | 186096 |
| 7250. | M97935 | Genbank | 2281070 |
| 7251. | S39127 | Genbank | 250802 |
| 7252. | S49006 | Genbank | 260617 |
| 7253. | S53268 | Genbank | 234745 |
| 7254. | S54005 | Genbank | 264772 |
| 7255. | S54641 | Genbank | 265483 |
| 7256. | S54761 | Genbank | 265221 |
| 7257. | S56449 | Genbank | 266242 |
| 7258. | S73591 | Genbank | 688296 |
| 7259. | S75725 | Genbank | 861520 |
| 7260. | S75755 | Genbank | 861469 |
| 7261. | S77601 | Genbank | 998394 |
| 7262. | S79522 | Genbank | 243887 |
| 7263. | S79895 | Genbank | 1195555 |
| 7264. | S81003 | Genbank | 1488376 |
| 7265. | S81752 | Genbank | 1438780 |
| 7266. | S82081 | Genbank | 1488412 |
| 7267. | S82297 | Genbank | 245387 |
| 7268. | S82451 | Genbank | 1699299 |
| 7269. | S82496 | Genbank | 1699161 |
| 7270. | U02619 | Genbank | 414932 |
| 7271. | U03105 | Genbank | 476094 |
| 7272. | U04627 | Genbank | 595266 |
| 7273. | U05875 | Genbank | 463549 |
| 7274. | U07231 | Genbank | 517195 |
| 7275. | U07550 | Genbank | 469170 |
| 7276. | U07802 | Genbank | 984508 |
| 7277. | U07919 | Genbank | 995897 |
| 7278. | U08023 | Genbank | 505664 |
| 7279. | U09117 | Genbank | 483919 |
| 7280. | U09813 | Genbank | 1008454 |
| 7281. | U10087 | Genbank | 1226232 |
| 7282. | U10248 | Genbank | 984280 |
| 7283. | U10323 | Genbank | 532312 |
| 7284. | U10360 | Genbank | 551490 |
| 7285. | U10485 | Genbank | 505685 |
| 7286. | U11276 | Genbank | 538270 |
| 7287. | U12404 | Genbank | 531170 |
| 7288. | U12465 | Genbank | 562073 |
| 7289. | U12535 | Genbank | 530822 |
| 7290. | U13616 | Genbank | 608024 |
| 7291. | U13665 | Genbank | 606922 |
| 7292. | U14394 | Genbank | 608128 |
| 7293. | U14967 | Genbank | 550014 |
| 7294. | U14969 | Genbank | 550018 |
| 7295. | U14970 | Genbank | 550020 |
| 7296. | U14971 | Genbank | 550022 |
| 7297. | U14972 | Genbank | 550024 |
| 7298. | U15008 | Genbank | 600747 |
| 7299. | U16660 | Genbank | 564064 |
| 7300. | U19523 | Genbank | 755461 |
| 7301. | U20998 | Genbank | 897850 |
| 7302. | U25816 | Genbank | 837262 |
| 7303. | U27109 | Genbank | 927595 |
| 7304. | U27143 | Genbank | 862932 |
| 7305. | U27460 | Genbank | 881393 |
| 7306. | U29953 | Genbank | 1144298 |
| 7307. | U30897 | Genbank | 2076716 |
| 7308. | U31525 | Genbank | 976399 |
| 7309. | U31814 | Genbank | 1667393 |
| 7310. | U31906 | Genbank | 1173564 |
| 7311. | U32515 | Genbank | 1262186 |
| 7312. | U34044 | Genbank | 1000283 |
| 7313. | U34070 | Genbank | 1041732 |
| 7314. | U34877 | Genbank | 1143231 |
| 7315. | U35143 | Genbank | 1016272 |
| 7316. | U35612 | Genbank | 2341055 |
| 7317. | U37518 | Genbank | 1149557 |
| 7318. | U38784 | Genbank | 1574947 |
| 7319. | U38817 | Genbank | 1401054 |
| 7320. | U39050 | Genbank | 1063685 |
| 7321. | U39360 | Genbank | 1066079 |
| 7322. | U39945 | Genbank | 1209686 |
| 7323. | U40268 | Genbank | 4433811 |
| 7324. | U40992 | Genbank | 6031211 |
| 7325. | U41514 | Genbank | 1136284 |
| 7326. | U41515 | Genbank | 1209723 |
| 7327. | U41668 | Genbank | 1477481 |
| 7328. | U42360 | Genbank | 1353698 |
| 7329. | U42361 | Genbank | 4096843 |
| 7330. | U43286 | Genbank | 1815621 |
| 7331. | U43374 | Genbank | 1163082 |
| 7332. | U44772 | Genbank | 1314354 |
| 7333. | U46751 | Genbank | 3077821 |
| 7334. | U47674 | Genbank | 3860239 |
| 7335. | U47742 | Genbank | 1517913 |
| 7336. | U47924 | Genbank | 1633547 |
| 7337. | U49245 | Genbank | 1216503 |
| 7338. | U49957 | Genbank | 1537016 |
| 7339. | U50535 | Genbank | 1531607 |
| 7340. | U50939 | Genbank | 1314559 |
| 7341. | U51205 | Genbank | 1730283 |
| 7342. | U51678 | Genbank | 1915966 |
| 7343. | U51903 | Genbank | 1262925 |
| 7344. | U52522 | Genbank | 1279762 |
| 7345. | U53468 | Genbank | 1373172 |
| 7346. | U54559 | Genbank | 2351379 |
| 7347. | U54993 | Genbank | 4097314 |
| 7348. | U57693 | Genbank | 1373376 |
| 7349. | U57847 | Genbank | 1373420 |
| 7350. | U60800 | Genbank | 1663566 |
| 7351. | U61397 | Genbank | 1518693 |
| 7352. | U62392 | Genbank | 2315851 |
| 7353. | U62740 | Genbank | 1518041 |
| 7354. | U64820 | Genbank | 2262194 |
| 7355. | U65579 | Genbank | 1935055 |
| 7356. | U67616 | Genbank | 1572587 |
| 7357. | U68093 | Genbank | 1562497 |
| 7358. | U68105 | Genbank | 1562509 |
| 7359. | U68494 | Genbank | 1546096 |
| 7360. | U68758 | Genbank | 4097815 |
| 7361. | U69127 | Genbank | 1575608 |
| 7362. | U70063 | Genbank | 1743866 |
| 7363. | U70660 | Genbank | 1945364 |
| 7364. | U72788 | Genbank | 1575796 |
| 7365. | U73522 | Genbank | 4098123 |
| 7366. | U77396 | Genbank | 1684871 |
| 7367. | U77942 | Genbank | 2337919 |
| 7368. | U81006 | Genbank | 1737489 |
| 7369. | U83460 | Genbank | 2315986 |
| 7370. | U84371 | Genbank | 1813879 |
| 7371. | U85193 | Genbank | 1814408 |
| 7372. | U86602 | Genbank | 1835785 |
| 7373. | U88320 | Genbank | 2196919 |
| 7374. | U89336 | Genbank | 1841547 |
| 7375. | U89505 | Genbank | 2078528 |
| 7376. | U90304 | Genbank | 1899219 |
| 7377. | U90547 | Genbank | 2062695 |
| 7378. | U90916 | Genbank | 1913897 |
| 7379. | U90920 | Genbank | 2522321 |
| 7380. | U91326 | Genbank | 1931583 |
| 7381. | U91328 | Genbank | 2088550 |
| 7382. | U92980 | Genbank | 2781398 |
| 7383. | U94728 | Genbank | 2055423 |
| 7384. | U95218 | Genbank | 3420001 |
| 7385. | U97519 | Genbank | 2213812 |
| 7386. | V00478 | Genbank | 28244 |
| 7387. | V00518 | Genbank | 31868 |
| 7388. | V00531 | Genbank | 32631 |
| 7389. | X00351 | Genbank | 28251 |
| 7390. | X00457 | Genbank | 36405 |
| 7391. | X01037 | Genbank | 36086 |
| 7392. | X01451 | Genbank | 36774 |
| 7393. | X01630 | Genbank | 28871 |
| 7394. | X01742 | Genbank | 35324 |
| 7395. | X02152 | Genbank | 34312 |
| 7396. | X02422 | Genbank | 33344 |
| 7397. | X02530 | Genbank | 33917 |
| 7398. | X03205 | Genbank | 36162 |
| 7399. | X04098 | Genbank | 28338 |
| 7400. | X04327 | Genbank | 29480 |
| 7401. | X04526 | Genbank | 31667 |
| 7402. | X04588 | Genbank | 37423 |
| 7403. | X04803 | Genbank | 6647297 |
| 7404. | X04828 | Genbank | 31743 |
| 7405. | X05236 | Genbank | 28596 |
| 7406. | X05332 | Genbank | 35740 |
| 7407. | X05857 | Genbank | 31988 |
| 7408. | X06882 | Genbank | 29736 |
| 7409. | X07109 | Genbank | 35492 |
| 7410. | X07369 | Genbank | 35319 |
| 7411. | X07549 | Genbank | 29707 |
| 7412. | X07743 | Genbank | 35517 |
| 7413. | X12597 | Genbank | 32326 |
| 7414. | X13238 | Genbank | 1200056 |
| 7415. | X13923 | Genbank | 30294 |
| 7416. | X14420 | Genbank | 30057 |
| 7417. | X15187 | Genbank | 37260 |
| 7418. | X15606 | Genbank | 32623 |
| 7419. | X15729 | Genbank | 38317 |
| 7420. | X15822 | Genbank | 30146 |
| 7421. | X16064 | Genbank | 37495 |
| 7422. | X17206 | Genbank | 34391 |
| 7423. | X51405 | Genbank | 29666 |
| 7424. | X51804 | Genbank | 35534 |
| 7425. | X52882 | Genbank | 311380 |
| 7426. | X52966 | Genbank | 34200 |
| 7427. | X53793 | Genbank | 28383 |
| 7428. | X54304 | Genbank | 34755 |
| 7429. | X54326 | Genbank | 31957 |
| 7430. | X54941 | Genbank | 29976 |
| 7431. | X55525 | Genbank | 30101 |
| 7432. | X55733 | Genbank | 288099 |
| 7433. | X56134 | Genbank | 37849 |
| 7434. | X56932 | Genbank | 23690 |
| 7435. | X57025 | Genbank | 33007 |
| 7436. | X57346 | Genbank | 23113 |
| 7437. | X57398 | Genbank | 35526 |
| 7438. | X57802 | Genbank | 33701 |
| 7439. | X57819 | Genbank | 33737 |
| 7440. | X59417 | Genbank | 35681 |
| 7441. | X60036 | Genbank | 38261 |
| 7442. | X60489 | Genbank | 31099 |
| 7443. | X60656 | Genbank | 31134 |
| 7444. | X60674 | Genbank | 28578 |
| 7445. | X61970 | Genbank | 296739 |
| 7446. | X62125 | Genbank | 38334 |
| 7447. | X62744 | Genbank | 36062 |
| 7448. | X63526 | Genbank | 31101 |
| 7449. | X64707 | Genbank | 29382 |
| 7450. | X67698 | Genbank | 37476 |
| 7451. | X67858 | Genbank | 33217 |
| 7452. | X67951 | Genbank | 287640 |
| 7453. | X70326 | Genbank | 38434 |
| 7454. | X70476 | Genbank | 298096 |
| 7455. | X72500 | Genbank | 298106 |
| 7456. | X73459 | Genbank | 313660 |
| 7457. | X74070 | Genbank | 395086 |
| 7458. | X74968 | Genbank | 439659 |
| 7459. | X75861 | Genbank | 456258 |
| 7460. | X78627 | Genbank | 607129 |
| 7461. | X79234 | Genbank | 495125 |
| 7462. | X80306 | Genbank | 515765 |
| 7463. | X80910 | Genbank | 531475 |
| 7464. | X81109 | Genbank | 535057 |
| 7465. | X81695 | Genbank | 940515 |
| 7466. | X81788 | Genbank | 1045058 |
| 7467. | X82153 | Genbank | 562756 |
| 7468. | X83218 | Genbank | 1008079 |
| 7469. | X83544 | Genbank | 1089849 |
| 7470. | X83973 | Genbank | 639692 |
| 7471. | X87689 | Genbank | 895844 |
| 7472. | X89401 | Genbank | 984142 |
| 7473. | X89593 | Genbank | 6630776 |
| 7474. | X89985 | Genbank | 929618 |
| 7475. | X90583 | Genbank | 1071680 |
| 7476. | X90840 | Genbank | 1212916 |
| 7477. | X98296 | Genbank | 1666074 |
| 7478. | Y00052 | Genbank | 30308 |
| 7479. | Y00062 | Genbank | 34275 |
| 7480. | Y00345 | Genbank | 35569 |
| 7481. | Y08132 | Genbank | 1707538 |
| 7482. | Y09328 | Genbank | 1885307 |
| 7483. | Y12781 | Genbank | 3021408 |
| 7484. | Y13286 | Genbank | 2853173 |
| 7485. | Y13620 | Genbank | 2181877 |
| 7486. | Y14155 | Genbank | 2251148 |
| 7487. | Y14737 | Genbank | 2765424 |
| 7488. | Y15286 | Genbank | 2584788 |
| 7489. | Y17293 | Genbank | 3821247 |
| 7490. | Y17957 | Genbank | 10241692 |
| 7491. | Z11692 | Genbank | 31107 |
| 7492. | Z11793 | Genbank | 36425 |
| 7493. | Z11890 | Genbank | 33202 |
| 7494. | Z11894 | Genbank | 33200 |
| 7495. | Z14244 | Genbank | 30150 |
| 7496. | Z29331 | Genbank | 483539 |
| 7497. | Z35227 | Genbank | 609016 |
| 7498. | Z47087 | Genbank | 860989 |
| 7499. | Z48950 | Genbank | 761715 |
| 7500. | Z50749 | Genbank | 1085027 |
| 7501. | Z74615 | Genbank | 1418927 |
| 7502. | Z78846 | Genbank | 1508124 |
| 7503. | Z80902 | Genbank | 6572412 |
| 7504. | Z82188 | Genbank | 5102611 |
| 7505. | Z82217 | Genbank | 2696013 |
| 7506. | Z83840 | Genbank | 4914518 |
| 7507. | Z83844 | Genbank | 4467204 |
| 7508. | Z84484 | Genbank | 2760548 |
| 7509. | Z84814 | Genbank | 1834458 |
| 7510. | Z85038 | Genbank | 1834749 |
| 7511. | Z85247 | Genbank | 1834958 |
| 7512. | Z85986 | Genbank | 4034056 |
| 7513. | Z85987 | Genbank | 5531490 |
| 7514. | Z85996 | Genbank | 2276311 |
| 7515. | Z93016 | Genbank | 9650703 |
| 7516. | Z94721 | Genbank | 2462374 |
| 7517. | Z95114 | Genbank | 5101742 |
| 7518. | Z97029 | Genbank | 3334760 |
| 7519. | Z97053 | Genbank | 9650676 |
| 7520. | Z97200 | Genbank | 3115994 |
| 7521. | Z97832 | Genbank | 6065887 |
| 7522. | Z98036 | Genbank | 2578066 |
| 7523. | Z98257 | Genbank | 2956660 |
| 7524. | Z98751 | Genbank | 2814366 |
| 7525. | Z99705 | Genbank | 2950206 |
| 7526. | AA001197 | EST | 1437282 |
| 7527. | AA004674 | EST | 1448211 |
| 7528. | AA005329 | EST | 1447881 |
| 7529. | AA009518 | EST | 1470717 |
| 7530. | AA009596 | EST | 1470755 |
| 7531. | AA009605 | EST | 1470746 |
| 7532. | AA009694 | EST | 1470557 |
| 7533. | AA010168 | EST | 1471344 |
| 7534. | AA010479 | EST | 1471525 |
| 7535. | AA010642 | EST | 1471688 |
| 7536. | AA010740 | EST | 1471835 |
| 7537. | AA011165 | EST | 1472192 |
| 7538. | AA011314 | EST | 1472361 |
| 7539. | AA015584 | EST | 1476650 |
| 7540. | AA015609 | EST | 1476657 |
| 7541. | AA017474 | EST | 1479639 |
| 7542. | AA018773 | EST | 1481451 |
| 7543. | AA019093 | EST | 1482610 |
| 7544. | AA019534 | EST | 1482153 |
| 7545. | AA019928 | EST | 1483782 |
| 7546. | AA022580 | EST | 1486670 |
| 7547. | AA022899 | EST | 1486988 |
| 7548. | AA024700 | EST | 1489613 |
| 7549. | AA024816 | EST | 1489730 |
| 7550. | AA025368 | EST | 1491343 |
| 7551. | AA025432 | EST | 1490914 |
| 7552. | AA026166 | EST | 1492637 |
| 7553. | AA026455 | EST | 1492355 |
| 7554. | AA026962 | EST | 1493153 |
| 7555. | AA027048 | EST | 1493238 |
| 7556. | AA029131 | EST | 1496533 |
| 7557. | AA029220 | EST | 1496624 |
| 7558. | AA031825 | EST | 1501788 |
| 7559. | AA032047 | EST | 1502019 |
| 7560. | AA033613 | EST | 1505441 |
| 7561. | AA033645 | EST | 1505473 |
| 7562. | AA034054 | EST | 1505863 |
| 7563. | AA034078 | EST | 1505905 |
| 7564. | AA034094 | EST | 1505921 |
| 7565. | AA034236 | EST | 1506264 |
| 7566. | AA035039 | EST | 1506982 |
| 7567. | AA036876 | EST | 1509951 |
| 7568. | AA037570 | EST | 1512670 |
| 7569. | AA037857 | EST | 1512993 |
| 7570. | AA039274 | EST | 1515552 |
| 7571. | AA039379 | EST | 1515796 |
| 7572. | AA039882 | EST | 1516225 |
| 7573. | AA040007 | EST | 1516311 |
| 7574. | AA041223 | EST | 1517457 |
| 7575. | AA041551 | EST | 1517804 |
| 7576. | AA043292 | EST | 1521146 |
| 7577. | AA043804 | EST | 1521717 |
| 7578. | AA044755 | EST | 1522958 |
| 7579. | AA044760 | EST | 1522963 |
| 7580. | AA045163 | EST | 1523365 |
| 7581. | AA045480 | EST | 1523696 |
| 7582. | AA045543 | EST | 1525306 |
| 7583. | AA046482 | EST | 1524475 |
| 7584. | AA047320 | EST | 1525354 |
| 7585. | AA047429 | EST | 1525475 |
| 7586. | AA047774 | EST | 1527444 |
| 7587. | AA053176 | EST | 1544385 |
| 7588. | AA053884 | EST | 1544855 |
| 7589. | AA055615 | EST | 1548161 |
| 7590. | AA055838 | EST | 1548240 |
| 7591. | AA056365 | EST | 1548705 |
| 7592. | AA056412 | EST | 1548897 |
| 7593. | AA056669 | EST | 1549009 |
| 7594. | AA058590 | EST | 1551397 |
| 7595. | AA062583 | EST | 1556851 |
| 7596. | AA063590 | EST | 1557557 |
| 7597. | AA069779 | EST | 1577147 |
| 7598. | AA070515 | EST | 1578012 |
| 7599. | AA071165 | EST | 1578526 |
| 7600. | AA071213 | EST | 1578593 |
| 7601. | AA074487 | EST | 1614538 |
| 7602. | AA075398 | EST | 1615269 |
| 7603. | AA075584 | EST | 1615453 |
| 7604. | AA075663 | EST | 1615533 |
| 7605. | AA076328 | EST | 1616197 |
| 7606. | AA077736 | EST | 1837224 |
| 7607. | AA079880 | EST | 1618772 |
| 7608. | AA081042 | EST | 1622977 |
| 7609. | AA081320 | EST | 1623125 |
| 7610. | AA082502 | EST | 1624683 |
| 7611. | AA082769 | EST | 1624844 |
| 7612. | AA082937 | EST | 1624994 |
| 7613. | AA083509 | EST | 1625569 |
| 7614. | AA083711 | EST | 1625771 |
| 7615. | AA084560 | EST | 1626616 |
| 7616. | AA085933 | EST | 1629355 |
| 7617. | AA088238 | EST | 1633750 |
| 7618. | AA088429 | EST | 1633949 |
| 7619. | AA086559 | EST | 1634064 |
| 7620. | AA088795 | EST | 1634341 |
| 7621. | AA089796 | EST | 1636288 |
| 7622. | AA090151 | EST | 1636667 |
| 7623. | AA092115 | EST | 1637112 |
| 7624. | AA093509 | EST | 1639094 |
| 7625. | AA099715 | EST | 1645807 |
| 7626. | AA099797 | EST | 1646670 |
| 7627. | AA099901 | EST | 1646047 |
| 7628. | AA102276 | EST | 1646687 |
| 7629. | AA113871 | EST | 1668073 |
| 7630. | AA114907 | EST | 1670119 |
| 7631. | AA115111 | EST | 1669959 |
| 7632. | AA115143 | EST | 1670567 |
| 7633. | AA125763 | EST | 1687753 |
| 7634. | AA126720 | EST | 1686238 |
| 7635. | AA126754 | EST | 1686254 |
| 7636. | AA127457 | EST | 1686792 |
| 7637. | AA127713 | EST | 1687002 |
| 7638. | AA128541 | EST | 1688513 |
| 7639. | AA128601 | EST | 1689996 |
| 7640. | AA129017 | EST | 1688906 |
| 7641. | AA129420 | EST | 1689185 |
| 7642. | AA130384 | EST | 1691527 |
| 7643. | AA130438 | EST | 1691721 |
| 7644. | AA131231 | EST | 1692739 |
| 7645. | AA131252 | EST | 1692814 |
| 7646. | AA131783 | EST | 1693309 |
| 7647. | AA131930 | EST | 1693428 |
| 7648. | AA132250 | EST | 1693849 |
| 7649. | AA135582 | EST | 1696613 |
| 7650. | AA135870 | EST | 1696844 |
| 7651. | AA136567 | EST | 1697777 |
| 7652. | AA143061 | EST | 1712449 |
| 7653. | AA143074 | EST | 1712578 |
| 7654. | AA146592 | EST | 1715983 |
| 7655. | AA146953 | EST | 1716325 |
| 7656. | AA147094 | EST | 1716467 |
| 7657. | AA147714 | EST | 1717086 |
| 7658. | AA147836 | EST | 1717208 |
| 7659. | AA148266 | EST | 1717664 |
| 7660. | AA149410 | EST | 1719926 |
| 7661. | AA149845 | EST | 1720925 |
| 7662. | AA150369 | EST | 1721900 |
| 7663. | AA150416 | EST | 1721929 |
| 7664. | AA151566 | EST | 1720184 |
| 7665. | AA151678 | EST | 1720233 |
| 7666. | AA151765 | EST | 1720479 |
| 7667. | AA155675 | EST | 1727481 |
| 7668. | AA155839 | EST | 1727457 |
| 7669. | AA155988 | EST | 1727606 |
| 7670. | AA156065 | EST | 1727699 |
| 7671. | AA156092 | EST | 1727708 |
| 7672. | AA157841 | EST | 1732652 |
| 7673. | AA158769 | EST | 1733571 |
| 7674. | AA159515 | EST | 1735058 |
| 7675. | AA160328 | EST | 1734907 |
| 7676. | AA160807 | EST | 1736174 |
| 7677. | AA164399 | EST | 1740577 |
| 7678. | AA164759 | EST | 1740937 |
| 7679. | AA165074 | EST | 1740302 |
| 7680. | AA167011 | EST | 1745386 |
| 7681. | AA167028 | EST | 1745403 |
| 7682. | AA167085 | EST | 1745461 |
| 7683. | AA167203 | EST | 1745770 |
| 7684. | AA169602 | EST | 1747990 |
| 7685. | AA172081 | EST | 1751139 |
| 7686. | AA173284 | EST | 1753416 |
| 7687. | AA173450 | EST | 1753599 |
| 7688. | AA176404 | EST | 1757553 |
| 7689. | AA179224 | EST | 1760576 |
| 7690. | AA179528 | EST | 1760905 |
| 7691. | AA181902 | EST | 1765379 |
| 7692. | AA186633 | EST | 1774732 |
| 7693. | AA187978 | EST | 1774425 |
| 7694. | AA190847 | EST | 1779493 |
| 7695. | AA191629 | EST | 1780293 |
| 7696. | AA192516 | EST | 1781738 |
| 7697. | AA194815 | EST | 1784818 |
| 7698. | AA194980 | EST | 1784901 |
| 7699. | AA195114 | EST | 1784804 |
| 7700. | AA195119 | EST | 1784830 |
| 7701. | AA197141 | EST | 1792715 |
| 7702. | AA203201 | EST | 1798911 |
| 7703. | AA203477 | EST | 1799204 |
| 7704. | AA203672 | EST | 1799391 |
| 7705. | AA205426 | EST | 1803478 |
| 7706. | AA206064 | EST | 1801436 |
| 7707. | AA214209 | EST | 1812828 |
| 7708. | AA214544 | EST | 1813169 |
| 7709. | AA215736 | EST | 1815480 |
| 7710. | AA219769 | EST | 1833844 |
| 7711. | AA224789 | EST | 1846213 |
| 7712. | AA225115 | EST | 1846489 |
| 7713. | AA225969 | EST | 1847268 |
| 7714. | AA227897 | EST | 1849441 |
| 7715. | AA228954 | EST | 1875048 |
| 7716. | AA229014 | EST | 1851979 |
| 7717. | AA229607 | EST | 1851604 |
| 7718. | AA232477 | EST | 1855264 |
| 7719. | AA233146 | EST | 1856141 |
| 7720. | AA234050 | EST | 1858191 |
| 7721. | AA234628 | EST | 1859148 |
| 7722. | AA234796 | EST | 1859289 |
| 7723. | AA236175 | EST | 1860605 |
| 7724. | AA236638 | EST | 1860658 |
| 7725. | AA237032 | EST | 1861125 |
| 7726. | AA243809 | EST | 1874620 |
| 7727. | AA251545 | EST | 1886509 |
| 7728. | AA251578 | EST | 1886542 |
| 7729. | AA252158 | EST | 1887121 |
| 7730. | AA253389 | EST | 1885694 |
| 7731. | AA255602 | EST | 1892449 |
| 7732. | AA256762 | EST | 1892526 |
| 7733. | AA258577 | EST | 1893701 |
| 7734. | AA259158 | EST | 1894593 |
| 7735. | AA279067 | EST | 1920532 |
| 7736. | AA280686 | EST | 1923391 |
| 7737. | AA280962 | EST | 1923643 |
| 7738. | AA281008 | EST | 1923752 |
| 7739. | AA281655 | EST | 1924558 |
| 7740. | AA281985 | EST | 1924809 |
| 7741. | AA282294 | EST | 1925210 |
| 7742. | AA284494 | EST | 1928811 |
| 7743. | AA284698 | EST | 1927389 |
| 7744. | AA285040 | EST | 1927721 |
| 7745. | AA285290 | EST | 1929600 |
| 7746. | AA291073 | EST | 1939180 |
| 7747. | AA291427 | EST | 1939465 |
| 7748. | AA291997 | EST | 1939974 |
| 7749. | AA292953 | EST | 1940848 |
| 7750. | AA298973 | EST | 1951305 |
| 7751. | AA300651 | EST | 1953221 |
| 7752. | AA305961 | EST | 1958290 |
| 7753. | AA306100 | EST | 1958428 |
| 7754. | AA306264 | EST | 1958592 |
| 7755. | AA306488 | EST | 1959058 |
| 7756. | AA306545 | EST | 1958874 |
| 7757. | AA307732 | EST | 1960060 |
| 7758. | AA309140 | EST | 1961465 |
| 7759. | AA310377 | EST | 1962706 |
| 7760. | AA312968 | EST | 1965595 |
| 7761. | AA314119 | EST | 1966448 |
| 7762. | AA314292 | EST | 1966621 |
| 7763. | AA314872 | EST | 1967221 |
| 7764. | AA315509 | EST | 1967858 |
| 7765. | AA315835 | EST | 1968184 |
| 7766. | AA316153 | EST | 1968502 |
| 7767. | AA316493 | EST | 1969063 |
| 7768. | AA316649 | EST | 1969037 |
| 7769. | AA328968 | EST | 1981211 |
| 7770. | AA341606 | EST | 1994025 |
| 7771. | AA343467 | EST | 1995726 |
| 7772. | AA345289 | EST | 1997545 |
| 7773. | AA348809 | EST | 2001046 |
| 7774. | AA353000 | EST | 2005320 |
| 7775. | AA363408 | EST | 2015728 |
| 7776. | AA364286 | EST | 2016604 |
| 7777. | AA371913 | EST | 2024294 |
| 7778. | AA373089 | EST | 2025643 |
| 7779. | AA397736 | EST | 2050627 |
| 7780. | AA397976 | EST | 2050688 |
| 7781. | AA399281 | EST | 2053016 |
| 7782. | AA400609 | EST | 2054480 |
| 7783. | AA405018 | EST | 2063340 |
| 7784. | AA410285 | EST | 2069246 |
| 7785. | AA411582 | EST | 2069115 |
| 7786. | AA412254 | EST | 2070824 |
| 7787. | AA418661 | EST | 2080480 |
| 7788. | AA421614 | EST | 2100430 |
| 7789. | AA422114 | EST | 2100938 |
| 7790. | AA424202 | EST | 2103207 |
| 7791. | AA424880 | EST | 2106985 |
| 7792. | AA425850 | EST | 2107670 |
| 7793. | AA428016 | EST | 2111744 |
| 7794. | AA428515 | EST | 2112512 |
| 7795. | AA428819 | EST | 2110370 |
| 7796. | AA429308 | EST | 2111921 |
| 7797. | AA429637 | EST | 2112738 |
| 7798. | AA429820 | EST | 2112993 |
| 7799. | AA430565 | EST | 2111149 |
| 7800. | AA431401 | EST | 2115109 |
| 7801. | AA431823 | EST | 2115531 |
| 7802. | AA431846 | EST | 2115554 |
| 7803. | AA436018 | EST | 2140932 |
| 7804. | AA436180 | EST | 2141094 |
| 7805. | AA436472 | EST | 2141386 |
| 7806. | AA437173 | EST | 2142087 |
| 7807. | AA442109 | EST | 2153987 |
| 7808. | AA442453 | EST | 2154331 |
| 7809. | AA443660 | EST | 2156335 |
| 7810. | AA446058 | EST | 2158723 |
| 7811. | AA446441 | EST | 2159106 |
| 7812. | AA446507 | EST | 2159172 |
| 7813. | AA449550 | EST | 2163300 |
| 7814. | AA449621 | EST | 2163371 |
| 7815. | AA449632 | EST | 2163382 |
| 7816. | AA449889 | EST | 2163639 |
| 7817. | AA452000 | EST | 2165669 |
| 7818. | AA452012 | EST | 2165681 |
| 7819. | AA453503 | EST | 2167172 |
| 7820. | AA454029 | EST | 2167698 |
| 7821. | AA454909 | EST | 2177685 |
| 7822. | AA456287 | EST | 2179497 |
| 7823. | AA458761 | EST | 2183668 |
| 7824. | AA459437 | EST | 2184344 |
| 7825. | AA459854 | EST | 2184761 |
| 7826. | AA460645 | EST | 2185765 |
| 7827. | AA460963 | EST | 2186083 |
| 7828. | AA461425 | EST | 2185289 |
| 7829. | AA465366 | EST | 2191533 |
| 7830. | AA468124 | EST | 2194658 |
| 7831. | AA468276 | EST | 2194810 |
| 7832. | AA469979 | EST | 2197288 |
| 7833. | AA470599 | EST | 2197908 |
| 7834. | AA478299 | EST | 2206933 |
| 7835. | AA479860 | EST | 2205746 |
| 7836. | AA480147 | EST | 2208298 |
| 7837. | AA481837 | EST | 2209515 |
| 7838. | AA483071 | EST | 2211916 |
| 7839. | AA485877 | EST | 2215096 |
| 7840. | AA486269 | EST | 2216485 |
| 7841. | AA489323 | EST | 2218925 |
| 7842. | AA489832 | EST | 2219364 |
| 7843. | AA492038 | EST | 2221600 |
| 7844. | AA494230 | EST | 2222887 |
| 7845. | AA501951 | EST | 2236918 |
| 7846. | AA502979 | EST | 2237946 |
| 7847. | AA503215 | EST | 2238182 |
| 7848. | AA504401 | EST | 2240561 |
| 7849. | AA505279 | EST | 2241416 |
| 7850. | AA505816 | EST | 2241953 |
| 7851. | AA507010 | EST | 2243449 |
| 7852. | AA507201 | EST | 2243640 |
| 7853. | AA507629 | EST | 2244068 |
| 7854. | AA507933 | EST | 2244372 |
| 7855. | AA513123 | EST | 2251535 |
| 7856. | AA514559 | EST | 2254159 |
| 7857. | AA515063 | EST | 2254663 |
| 7858. | AA515702 | EST | 2255302 |
| 7859. | AA522677 | EST | 2263389 |
| 7860. | AA522695 | EST | 2263407 |
| 7861. | AA523433 | EST | 2264145 |
| 7862. | AA523890 | EST | 2264818 |
| 7863. | AA524095 | EST | 2265023 |
| 7864. | AA526166 | EST | 2268235 |
| 7865. | AA526368 | EST | 2268437 |
| 7866. | AA527222 | EST | 2269291 |
| 7867. | AA527679 | EST | 2269748 |
| 7868. | AA527768 | EST | 2269837 |
| 7869. | AA531071 | EST | 2273777 |
| 7870. | AA534574 | EST | 2278827 |
| 7871. | AA535621 | EST | 2279874 |
| 7872. | AA548722 | EST | 2319004 |
| 7873. | AA551709 | EST | 2321961 |
| 7874. | AA552702 | EST | 2322956 |
| 7875. | AA554879 | EST | 2325418 |
| 7876. | AA554882 | EST | 2325421 |
| 7877. | AA555032 | EST | 2325571 |
| 7878. | AA557252 | EST | 2327729 |
| 7879. | AA557324 | EST | 2327801 |
| 7880. | AA559216 | EST | 2329412 |
| 7881. | AA563876 | EST | 2335515 |
| 7882. | AA563995 | EST | 2335634 |
| 7883. | AA564067 | EST | 2335706 |
| 7884. | AA566072 | EST | 2337711 |
| 7885. | AA569971 | EST | 2343951 |
| 7886. | AA570649 | EST | 2344629 |
| 7887. | AA576727 | EST | 2354201 |
| 7888. | AA577613 | EST | 2355087 |
| 7889. | AA577831 | EST | 2356015 |
| 7890. | AA584036 | EST | 2368645 |
| 7891. | AA587121 | EST | 2397935 |
| 7892. | AA588541 | EST | 2401716 |
| 7893. | AA588726 | EST | 2402457 |
| 7894. | AA588771 | EST | 2402502 |
| 7895. | AA594296 | EST | 2409646 |
| 7896. | AA594960 | EST | 2410310 |
| 7897. | AA595336 | EST | 2410686 |
| 7898. | AA599830 | EST | 2433455 |
| 7899. | AA603647 | EST | 2437508 |
| 7900. | AA604232 | EST | 2445141 |
| 7901. | AA604608 | EST | 2445472 |
| 7902. | AA608748 | EST | 2457176 |
| 7903. | AA608749 | EST | 2457177 |
| 7904. | AA608790 | EST | 2457218 |
| 7905. | AA609268 | EST | 2457696 |
| 7906. | AA609631 | EST | 2458059 |
| 7907. | AA610221 | EST | 2458649 |
| 7908. | AA610574 | EST | 2459002 |
| 7909. | AA612831 | EST | 2463869 |
| 7910. | AA614237 | EST | 2466371 |
| 7911. | AA614566 | EST | 2466762 |
| 7912. | AA634175 | EST | 2557389 |
| 7913. | AA643693 | EST | 2568911 |
| 7914. | AA650206 | EST | 2577534 |
| 7915. | AA650214 | EST | 2577542 |
| 7916. | AA653017 | EST | 2584669 |
| 7917. | AA654585 | EST | 2590739 |
| 7918. | AA665159 | EST | 2619772 |
| 7919. | AA665620 | EST | 2620233 |
| 7920. | AA677161 | EST | 2657683 |
| 7921. | AA678620 | EST | 2659142 |
| 7922. | AA679030 | EST | 2659552 |
| 7923. | AA679848 | EST | 2656315 |
| 7924. | AA682846 | EST | 2669529 |
| 7925. | AA687481 | EST | 2675672 |
| 7926. | AA687931 | EST | 2674837 |
| 7927. | AA688211 | EST | 2675117 |
| 7928. | AA693948 | EST | 2694886 |
| 7929. | AA700939 | EST | 2704104 |
| 7930. | AA701033 | EST | 2704198 |
| 7931. | AA702002 | EST | 2705115 |
| 7932. | AA702018 | EST | 2705131 |
| 7933. | AA722982 | EST | 2740689 |
| 7934. | AA723194 | EST | 2740971 |
| 7935. | AA731507 | EST | 2753663 |
| 7936. | AA732005 | EST | 2753956 |
| 7937. | AA737692 | EST | 2768449 |
| 7938. | AA741356 | EST | 2779948 |
| 7939. | AA743119 | EST | 2782625 |
| 7940. | AA746240 | EST | 2786226 |
| 7941. | AA748711 | EST | 2788669 |
| 7942. | AA748791 | EST | 2788749 |
| 7943. | AA757004 | EST | 2804867 |
| 7944. | AA758228 | EST | 2806091 |
| 7945. | AA764734 | EST | 2815972 |
| 7946. | AA765345 | EST | 2816583 |
| 7947. | AA766985 | EST | 2819566 |
| 7948. | AA770537 | EST | 2821775 |
| 7949. | AA772127 | EST | 2823910 |
| 7950. | AA772159 | EST | 2823942 |
| 7951. | AA773350 | EST | 2824921 |
| 7952. | AA775449 | EST | 2834783 |
| 7953. | AA777491 | EST | 2836970 |
| 7954. | AA806272 | EST | 2875022 |
| 7955. | AA807519 | EST | 2875586 |
| 7956. | AA808719 | EST | 2878125 |
| 7957. | AA810250 | EST | 2879609 |
| 7958. | AA811365 | EST | 2880976 |
| 7959. | AA811830 | EST | 2881441 |
| 7960. | AA825727 | EST | 2899039 |
| 7961. | AA828157 | EST | 2900520 |
| 7962. | AA829128 | EST | 2902227 |
| 7963. | AA829414 | EST | 2902513 |
| 7964. | AA832372 | EST | 2905471 |
| 7965. | AA835801 | EST | 2910120 |
| 7966. | AA843137 | EST | 2929655 |
| 7967. | AA843197 | EST | 2929715 |
| 7968. | AA844277 | EST | 2930728 |
| 7969. | AA845550 | EST | 2933309 |
| 7970. | AA854340 | EST | 2941878 |
| 7971. | AA856697 | EST | 2944999 |
| 7972. | AA873459 | EST | 2969581 |
| 7973. | AA873539 | EST | 2969661 |
| 7974. | AA877636 | EST | 2986601 |
| 7975. | AA877877 | EST | 2986842 |
| 7976. | AA878116 | EST | 2987081 |
| 7977. | AA878376 | EST | 2987341 |
| 7978. | AA887285 | EST | 3002393 |
| 7979. | AA890327 | EST | 3017206 |
| 7980. | AA894740 | EST | 3031141 |
| 7981. | AA902835 | EST | 3037958 |
| 7982. | AA905102 | EST | 3040225 |
| 7983. | AA916271 | EST | 3055663 |
| 7984. | AA916434 | EST | 3055826 |
| 7985. | AA918621 | EST | 3058511 |
| 7986. | AA922030 | EST | 3069339 |
| 7987. | AA937302 | EST | 3095413 |
| 7988. | AA946724 | EST | 3110119 |
| 7989. | AA953123 | EST | 3117270 |
| 7990. | AA971510 | EST | 3146800 |
| 7991. | AA983273 | EST | 3161798 |
| 7992. | AA985060 | EST | 3163585 |
| 7993. | AA991288 | EST | 3177777 |
| 7994. | AA993268 | EST | 3179813 |
| 7995. | AA995351 | EST | 3181840 |
| 7996. | AA996032 | EST | 3182521 |
| 7997. | AF109302 | EST | 6782697 |
| 7998. | AI004627 | EST | 3214137 |
| 7999. | AI014626 | EST | 3229007 |
| 8000. | AI016806 | EST | 3231142 |
| 8001. | AI018201 | EST | 3232720 |
| 8002. | AI018628 | EST | 3233147 |
| 8003. | AI022462 | EST | 3237703 |
| 8004. | AI027500 | EST | 3246430 |
| 8005. | AI033685 | EST | 3254638 |
| 8006. | AI034371 | EST | 3255324 |
| 8007. | AI038569 | EST | 3277763 |
| 8008. | AI064904 | EST | 6359176 |
| 8009. | AI065088 | EST | 6359360 |
| 8010. | AI065139 | EST | 6359411 |
| 8011. | AI066739 | EST | 3367025 |
| 8012. | AI074350 | EST | 3400994 |
| 8013. | AI075068 | EST | 3399848 |
| 8014. | AI076593 | EST | 3405771 |
| 8015. | AI078851 | EST | 3411663 |
| 8016. | AI080007 | EST | 3416258 |
| 8017. | AI081740 | EST | 3418532 |
| 8018. | AI083696 | EST | 3422119 |
| 8019. | AI085036 | EST | 3423459 |
| 8020. | AI090461 | EST | 3429520 |
| 8021. | AI090805 | EST | 3429864 |
| 8022. | AI091131 | EST | 3430190 |
| 8023. | AI096685 | EST | 3446179 |
| 8024. | AI114630 | EST | 6359975 |
| 8025. | AI122887 | EST | 3538653 |
| 8026. | AI127795 | EST | 3596309 |
| 8027. | AI133475 | EST | 6360791 |
| 8028. | AI140748 | EST | 3648205 |
| 8029. | AI143557 | EST | 3665366 |
| 8030. | AI146265 | EST | 3673947 |
| 8031. | AI146343 | EST | 3674025 |
| 8032. | AI148065 | EST | 3675747 |
| 8033. | AI151434 | EST | 3679903 |
| 8034. | AI160954 | EST | 3694259 |
| 8035. | AI167693 | EST | 3700863 |
| 8036. | AI168619 | EST | 3701789 |
| 8037. | AI174394 | EST | 3721247 |
| 8038. | AI174693 | EST | 6361071 |
| 8039. | AI188506 | EST | 3739715 |
| 8040. | AI192664 | EST | 3743873 |
| 8041. | AI204353 | EST | 3756959 |
| 8042. | AI207527 | EST | 6361535 |
| 8043. | AI218600 | EST | 3798415 |
| 8044. | AI219042 | EST | 3801245 |
| 8045. | AI219477 | EST | 3801680 |
| 8046. | AI220445 | EST | 3802648 |
| 8047. | AI224992 | EST | 3807705 |
| 8048. | AI241744 | EST | 3837141 |
| 8049. | AI243595 | EST | 3838992 |
| 8050. | AI245007 | EST | 3840404 |
| 8051. | AI246419 | EST | 3841816 |
| 8052. | AI247293 | EST | 3842690 |
| 8053. | AI247963 | EST | 3843360 |
| 8054. | AI249008 | EST | 3844405 |
| 8055. | AI249187 | EST | 3844584 |
| 8056. | AI249323 | EST | 3845852 |
| 8057. | AI254731 | EST | 3862256 |
| 8058. | AI261630 | EST | 3869833 |
| 8059. | AI262772 | EST | 3870975 |
| 8060. | AI264803 | EST | 3873006 |
| 8061. | AI269205 | EST | 3888372 |
| 8062. | AI269580 | EST | 3888747 |
| 8063. | AI270707 | EST | 3889874 |
| 8064. | AI273183 | EST | 3895451 |
| 8065. | AI274508 | EST | 3896776 |
| 8066. | AI274922 | EST | 3897196 |
| 8067. | AI275045 | EST | 3897319 |
| 8068. | AI277008 | EST | 3899276 |
| 8069. | AI279852 | EST | 3918086 |
| 8070. | AI281837 | EST | 3920070 |
| 8071. | AI282281 | EST | 3920514 |
| 8072. | AI288114 | EST | 3930891 |
| 8073. | AI288748 | EST | 3932251 |
| 8074. | AI298674 | EST | 3958410 |
| 8075. | AI302102 | EST | 3961448 |
| 8076. | AI307210 | EST | 4001966 |
| 8077. | AI310231 | EST | 4005102 |
| 8078. | AI310592 | EST | 4005463 |
| 8079. | AI312542 | EST | 4018147 |
| 8080. | AI332597 | EST | 4069156 |
| 8081. | AI338048 | EST | 4074975 |
| 8082. | AI338338 | EST | 4075265 |
| 8083. | AI341838 | EST | 4078765 |
| 8084. | AI343480 | EST | 4080686 |
| 8085. | AI348026 | EST | 4085232 |
| 8086. | AI354283 | EST | 4094436 |
| 8087. | AI355336 | EST | 4095489 |
| 8088. | AI357940 | EST | 4109561 |
| 8089. | AI358226 | EST | 4109847 |
| 8090. | AI358784 | EST | 4110405 |
| 8091. | AI358910 | EST | 4110531 |
| 8092. | AI360145 | EST | 4111766 |
| 8093. | AI368943 | EST | 4147696 |
| 8094. | AI369209 | EST | 4147962 |
| 8095. | AI371024 | EST | 4149777 |
| 8096. | AI373037 | EST | 4152903 |
| 8097. | AI374615 | EST | 4174605 |
| 8098. | AI377021 | EST | 4186874 |
| 8099. | AI379646 | EST | 4189499 |
| 8100. | AI379920 | EST | 4189773 |
| 8101. | AI380549 | EST | 4190402 |
| 8102. | AI380932 | EST | 4190785 |
| 8103. | AI393043 | EST | 4222590 |
| 8104. | AI418024 | EST | 4263955 |
| 8105. | AI432462 | EST | 4281890 |
| 8106. | AI433021 | EST | 4286257 |
| 8107. | AI434809 | EST | 4298775 |
| 8108. | AI435826 | EST | 4305899 |
| 8109. | AI436456 | EST | 4281845 |
| 8110. | AI439089 | EST | 4301881 |
| 8111. | AI440263 | EST | 4281427 |
| 8112. | AI445131 | EST | 4286848 |
| 8113. | AI446373 | EST | 4294755 |
| 8114. | AI479221 | EST | 4372389 |
| 8115. | AI479671 | EST | 4372839 |
| 8116. | AI500659 | EST | 4392641 |
| 8117. | AI510700 | EST | 4409605 |
| 8118. | AI523763 | EST | 4437898 |
| 8119. | AI525758 | EST | 4439893 |
| 8120. | AI539578 | EST | 4453713 |
| 8121. | AI557059 | EST | 4489422 |
| 8122. | AI559486 | EST | 4509691 |
| 8123. | AI560904 | EST | 4511245 |
| 8124. | AI561151 | EST | 4511492 |
| 8125. | AI567351 | EST | 4525803 |
| 8126. | AI567993 | EST | 4526445 |
| 8127. | AI568925 | EST | 4532299 |
| 8128. | AI570786 | EST | 4534160 |
| 8129. | AI572617 | EST | 4535991 |
| 8130. | AI572686 | EST | 4536060 |
| 8131. | AI581511 | EST | 4567408 |
| 8132. | AI613096 | EST | 4622263 |
| 8133. | AI613492 | EST | 4622659 |
| 8134. | AI634808 | EST | 4686138 |
| 8135. | AI635287 | EST | 4686617 |
| 8136. | AI638096 | EST | 4690330 |
| 8137. | AI640551 | EST | 4703660 |
| 8138. | AI650376 | EST | 4734355 |
| 8139. | AI658914 | EST | 4762484 |
| 8140. | AI659386 | EST | 4762956 |
| 8141. | AI660061 | EST | 4763631 |
| 8142. | AI668938 | EST | 4833712 |
| 8143. | AI672424 | EST | 4852155 |
| 8144. | AI674618 | EST | 4875098 |
| 8145. | AI675094 | EST | 4875574 |
| 8146. | AI675783 | EST | 4876263 |
| 8147. | AI681676 | EST | 4891858 |
| 8148. | AI682779 | EST | 4892961 |
| 8149. | AI684877 | EST | 4896171 |
| 8150. | AI686597 | EST | 4897891 |
| 8151. | AI689640 | EST | 4900934 |
| 8152. | AI690702 | EST | 4902004 |
| 8153. | AI693263 | EST | 4970603 |
| 8154. | AI693459 | EST | 4970799 |
| 8155. | AI694320 | EST | 4971660 |
| 8156. | AI696809 | EST | 4984709 |
| 8157. | AI697030 | EST | 4984930 |
| 8158. | AI697660 | EST | 4985571 |
| 8159. | AI697873 | EST | 4985773 |
| 8160. | AI699484 | EST | 4987384 |
| 8161. | AI700092 | EST | 4987992 |
| 8162. | AI701137 | EST | 4989037 |
| 8163. | AI702169 | EST | 4990069 |
| 8164. | AI702645 | EST | 4990545 |
| 8165. | AI741162 | EST | 5109450 |
| 8166. | AI742420 | EST | 5110708 |
| 8167. | AI744514 | EST | 5112802 |
| 8168. | AI751158 | EST | 5129422 |
| 8169. | AI753576 | EST | 5131840 |
| 8170. | AI753623 | EST | 5131887 |
| 8171. | AI758869 | EST | 5152594 |
| 8172. | AI760357 | EST | 5176024 |
| 8173. | AI767750 | EST | 5234259 |
| 8174. | AI783530 | EST | 5325339 |
| 8175. | AI795996 | EST | 5361459 |
| 8176. | AI803205 | EST | 5368677 |
| 8177. | AI805082 | EST | 5391648 |
| 8178. | AI808313 | EST | 5394879 |
| 8179. | AI815494 | EST | 5431040 |
| 8180. | AI816004 | EST | 5431550 |
| 8181. | AI820745 | EST | 5439824 |
| 8182. | AI824566 | EST | 5445237 |
| 8183. | AI827550 | EST | 5448221 |
| 8184. | AI829721 | EST | 5450392 |
| 8185. | AI862132 | EST | 5526239 |
| 8186. | AI863067 | EST | 5527174 |
| 8187. | AI867036 | EST | 5531143 |
| 8188. | AI868740 | EST | 5542718 |
| 8189. | AI869367 | EST | 5543335 |
| 8190. | AI874151 | EST | 5548200 |
| 8191. | AI879185 | EST | 5553234 |
| 8192. | AI879221 | EST | 5553270 |
| 8193. | AI884672 | EST | 5589836 |
| 8194. | AI885490 | EST | 5590654 |
| 8195. | AI887288 | EST | 5592452 |
| 8196. | AI904980 | EST | 6495367 |
| 8197. | AI905070 | EST | 6495457 |
| 8198. | AI905477 | EST | 6495864 |
| 8199. | AI908407 | EST | 6499087 |
| 8200. | AI909118 | EST | 6499798 |
| 8201. | AI912264 | EST | 5632119 |
| 8202. | AI917586 | EST | 5637441 |
| 8203. | AI919058 | EST | 5638913 |
| 8204. | AI923257 | EST | 5659221 |
| 8205. | AI925156 | EST | 5661120 |
| 8206. | AI925357 | EST | 5661321 |
| 8207. | AI926041 | EST | 5662005 |
| 8208. | AI928802 | EST | 5664795 |
| 8209. | AI929657 | EST | 5665621 |
| 8210. | AI932311 | EST | 5671048 |
| 8211. | AI954959 | EST | 5747269 |
| 8212. | AI963275 | EST | 5755988 |
| 8213. | AI971137 | EST | 5767963 |
| 8214. | AI971785 | EST | 5768611 |
| 8215. | AI984360 | EST | 5811637 |
| 8216. | AI989439 | EST | 5836243 |
| 8217. | AL035803 | EST | 5405440 |
| 8218. | AL036115 | EST | 5927646 |
| 8219. | AL036165 | EST | 5927669 |
| 8220. | AL041380 | EST | 5420731 |
| 8221. | AL042114 | EST | 5421459 |
| 8222. | AL042732 | EST | 5422181 |
| 8223. | AL043248 | EST | 5935832 |
| 8224. | AL045546 | EST | 5433677 |
| 8225. | AL046836 | EST | 5936250 |
| 8226. | AL047150 | EST | 5435180 |
| 8227. | AL047767 | EST | 4727955 |
| 8228. | AL047880 | EST | 4728068 |
| 8229. | AL047958 | EST | 4728791 |
| 8230. | AL047968 | EST | 4728801 |
| 8231. | AL048311 | EST | 4727451 |
| 8232. | AL048386 | EST | 4727526 |
| 8233. | AL079895 | EST | 5936639 |
| 8234. | AL079982 | EST | 5435556 |
| 8235. | AL119257 | EST | 5925156 |
| 8236. | AL121328 | EST | 5927329 |
| 8237. | AL135003 | EST | 6603190 |
| 8238. | AL135169 | EST | 6603356 |
| 8239. | AV646693 | EST | 9867707 |
| 8240. | AV646708 | EST | 9867722 |
| 8241. | AV646747 | EST | 9867761 |
| 8242. | AV648085 | EST | 9869099 |
| 8243. | AV648142 | EST | 9869156 |
| 8244. | AV650952 | EST | 9871966 |
| 8245. | AV652020 | EST | 9873034 |
| 8246. | AV653875 | EST | 9874889 |
| 8247. | AV655169 | EST | 9876183 |
| 8248. | AV658313 | EST | 9879327 |
| 8249. | AV658478 | EST | 9879492 |
| 8250. | AV681961 | EST | 10283824 |
| 8251. | AW006446 | EST | 5855224 |
| 8252. | AW007915 | EST | 5856693 |
| 8253. | AW015771 | EST | 5864528 |
| 8254. | AW019925 | EST | 5873455 |
| 8255. | AW021091 | EST | 5874621 |
| 8256. | AW021454 | EST | 5874984 |
| 8257. | AW025001 | EST | 5878531 |
| 8258. | AW025765 | EST | 5879295 |
| 8259. | AW026204 | EST | 5879723 |
| 8260. | AW055289 | EST | 5920992 |
| 8261. | AW063617 | EST | 8887554 |
| 8262. | AW081690 | EST | 6036842 |
| 8263. | AW088025 | EST | 6043830 |
| 8264. | AW089689 | EST | 6047033 |
| 8265. | AW104145 | EST | 6074880 |
| 8266. | AW104724 | EST | 6075459 |
| 8267. | AW104819 | EST | 6075554 |
| 8268. | AW137495 | EST | 6141813 |
| 8269. | AW138881 | EST | 6143199 |
| 8270. | AW148579 | EST | 6196475 |
| 8271. | AW149495 | EST | 6197391 |
| 8272. | AW152264 | EST | 6200164 |
| 8273. | AW161377 | EST | 6300410 |
| 8274. | AW170193 | EST | 6401707 |
| 8275. | AW170553 | EST | 6402078 |
| 8276. | AW172466 | EST | 6438414 |
| 8277. | AW172722 | EST | 6438670 |
| 8278. | AW188491 | EST | 6462927 |
| 8279. | AW195722 | EST | 6474914 |
| 8280. | AW195762 | EST | 6474966 |
| 8281. | AW241861 | EST | 6575615 |
| 8282. | AW242732 | EST | 6576577 |
| 8283. | AW291106 | EST | 6697742 |
| 8284. | AW291767 | EST | 6698403 |
| 8285. | AW292757 | EST | 6699393 |
| 8286. | AW297004 | EST | 6703640 |
| 8287. | AW327357 | EST | 6797852 |
| 8288. | AW327450 | EST | 6797945 |
| 8289. | AW328098 | EST | 6798594 |
| 8290. | AW339672 | EST | 6836298 |
| 8291. | AW361153 | EST | 6865803 |
| 8292. | AW362330 | EST | 6866950 |
| 8293. | AW363484 | EST | 6868134 |
| 8294. | AW365229 | EST | 6869879 |
| 8295. | AW368358 | EST | 6873008 |
| 8296. | AW371251 | EST | 6875905 |
| 8297. | AW374051 | EST | 6878705 |
| 8298. | AW382167 | EST | 6886826 |
| 8299. | AW388052 | EST | 6892620 |
| 8300. | AW391787 | EST | 6896446 |
| 8301. | AW393541 | EST | 6898200 |
| 8302. | AW401602 | EST | 6920288 |
| 8303. | AW404447 | EST | 6923504 |
| 8304. | AW404726 | EST | 6923783 |
| 8305. | AW404795 | EST | 6923852 |
| 8306. | AW405198 | EST | 6924255 |
| 8307. | AW405207 | EST | 6924264 |
| 8308. | AW405787 | EST | 6924844 |
| 8309. | AW405817 | EST | 6924874 |
| 8310. | AW405972 | EST | 6925029 |
| 8311. | AW406330 | EST | 6925387 |
| 8312. | AW406384 | EST | 6925441 |
| 8313. | AW473579 | EST | 7043685 |
| 8314. | AW500909 | EST | 7113948 |
| 8315. | AW501476 | EST | 7115041 |
| 8316. | AW512227 | EST | 7150305 |
| 8317. | AW514672 | EST | 7152754 |
| 8318. | AW517187 | EST | 7155269 |
| 8319. | AW518693 | EST | 7156699 |
| 8320. | AW518923 | EST | 7157005 |
| 8321. | AW575798 | EST | 7247337 |
| 8322. | AW579065 | EST | 7254114 |
| 8323. | AW579163 | EST | 7254212 |
| 8324. | AW581720 | EST | 7256769 |
| 8325. | AW602770 | EST | 7307511 |
| 8326. | AW607249 | EST | 7312094 |
| 8327. | AW608043 | EST | 7312784 |
| 8328. | AW613560 | EST | 7318746 |
| 8329. | AW629819 | EST | 7376609 |
| 8330. | AW629844 | EST | 7376634 |
| 8331. | AW630198 | EST | 7376988 |
| 8332. | AW754479 | EST | 7669411 |
| 8333. | AW770132 | EST | 7702171 |
| 8334. | AW770207 | EST | 7702270 |
| 8335. | AW798194 | EST | 7850168 |
| 8336. | AW801867 | EST | 7853737 |
| 8337. | AW818125 | EST | 7911119 |
| 8338. | AW819542 | EST | 7912536 |
| 8339. | AW827150 | EST | 7920846 |
| 8340. | AW875830 | EST | 8013776 |
| 8341. | AW878774 | EST | 8040784 |
| 8342. | AW936014 | EST | 8111420 |
| 8343. | AW949732 | EST | 8139367 |
| 8344. | AW949954 | EST | 8139594 |
| 8345. | AW951215 | EST | 8140883 |
| 8346. | AW951985 | EST | 8141664 |
| 8347. | AW952445 | EST | 8142142 |
| 8348. | AW953825 | EST | 8143628 |
| 8349. | AW954311 | EST | 8143994 |
| 8350. | AW954850 | EST | 8144533 |
| 8351. | AW955125 | EST | 8144808 |
| 8352. | AW956316 | EST | 8145999 |
| 8353. | AW957984 | EST | 8147667 |
| 8354. | AW958015 | EST | 8147698 |
| 8355. | AW959381 | EST | 8149065 |
| 8356. | AW960065 | EST | 8149749 |
| 8357. | AW960470 | EST | 8150154 |
| 8358. | AW961797 | EST | 8151483 |
| 8359. | AW963044 | EST | 8152880 |
| 8360. | AW964495 | EST | 8154331 |
| 8361. | AW964530 | EST | 8154366 |
| 8362. | AW965824 | EST | 8155660 |
| 8363. | AW966633 | EST | 8156469 |
| 8364. | AW967129 | EST | 8156965 |
| 8365. | AW967351 | EST | 8157188 |
| 8366. | AW967783 | EST | 8157622 |
| 8367. | AW969112 | EST | 8158953 |
| 8368. | AW974542 | EST | 8165742 |
| 8369. | AW977782 | EST | 8168932 |
| 8370. | AW978309 | EST | 8169573 |
| 8371. | AW993264 | EST | 8253415 |
| 8372. | AW997560 | EST | 8257794 |
| 8373. | BE002012 | EST | 8262245 |
| 8374. | BE002677 | EST | 8262910 |
| 8375. | BE010930 | EST | 8271163 |
| 8376. | BE044283 | EST | 8361336 |
| 8377. | BE044400 | EST | 8361453 |
| 8378. | BE047339 | EST | 8364392 |
| 8379. | BE047738 | EST | 8364791 |
| 8380. | BE061504 | EST | 8406154 |
| 8381. | BE080127 | EST | 8470414 |
| 8382. | BE080383 | EST | 8470670 |
| 8383. | BE084693 | EST | 8475048 |
| 8384. | BE091215 | EST | 8481667 |
| 8385. | BE092955 | EST | 8483407 |
| 8386. | BE147912 | EST | 8610636 |
| 8387. | BE161780 | EST | 8624501 |
| 8388. | BE218125 | EST | 8905372 |
| 8389. | BE222972 | EST | 8910290 |
| 8390. | BE241616 | EST | 9093339 |
| 8391. | BE250168 | EST | 9120275 |
| 8392. | BE251014 | EST | 9121133 |
| 8393. | BE253379 | EST | 9123534 |
| 8394. | BE253785 | EST | 9124206 |
| 8395. | BE255966 | EST | 9126417 |
| 8396. | BE258873 | EST | 9129368 |
| 8397. | BE259326 | EST | 9129827 |
| 8398. | BE261844 | EST | 9134361 |
| 8399. | BE262386 | EST | 9135359 |
| 8400. | BE262748 | EST | 9136120 |
| 8401. | BE265237 | EST | 9138802 |
| 8402. | BE265426 | EST | 9138994 |
| 8403. | BE266369 | EST | 9139945 |
| 8404. | BE267154 | EST | 9140742 |
| 8405. | BE267562 | EST | 9141155 |
| 8406. | BE268593 | EST | 9142200 |
| 8407. | BE269388 | EST | 9143008 |
| 8408. | BE269786 | EST | 9143413 |
| 8409. | BE270073 | EST | 9143703 |
| 8410. | BE274196 | EST | 9149136 |
| 8411. | BE274676 | EST | 9149620 |
| 8412. | BE277616 | EST | 9152587 |
| 8413. | BE297662 | EST | 9181242 |
| 8414. | BE311800 | EST | 9148368 |
| 8415. | BE314266 | EST | 9135185 |
| 8416. | BE315514 | EST | 9146407 |
| 8417. | BE315533 | EST | 9146455 |
| 8418. | BE328696 | EST | 9202472 |
| 8419. | BE348754 | EST | 9260607 |
| 8420. | BE350991 | EST | 9262772 |
| 8421. | BE378712 | EST | 9324077 |
| 8422. | BE380016 | EST | 9325381 |
| 8423. | BE390367 | EST | 9335732 |
| 8424. | BE392209 | EST | 9337574 |
| 8425. | BE392746 | EST | 9338098 |
| 8426. | BE393360 | EST | 9338821 |
| 8427. | BE393709 | EST | 9339061 |
| 8428. | BE407448 | EST | 9343898 |
| 8429. | BE409202 | EST | 9345652 |
| 8430. | BE439578 | EST | 9439060 |
| 8431. | BE463708 | EST | 9509483 |
| 8432. | BE467469 | EST | 9513244 |
| 8433. | BE503820 | EST | 9706228 |
| 8434. | BE513907 | EST | 9721119 |
| 8435. | BE531073 | EST | 9759718 |
| 8436. | BE536009 | EST | 9764654 |
| 8437. | BE537329 | EST | 9765974 |
| 8438. | BE539551 | EST | 9768196 |
| 8439. | BE543220 | EST | 9771865 |
| 8440. | BE549459 | EST | 9778104 |
| 8441. | BE549671 | EST | 9791363 |
| 8442. | BE562037 | EST | 9805757 |
| 8443. | BE564779 | EST | 9808499 |
| 8444. | BE565183 | EST | 9808903 |
| 8445. | BE565516 | EST | 9809236 |
| 8446. | BE565858 | EST | 9809578 |
| 8447. | BE567133 | EST | 9810853 |
| 8448. | BE568236 | EST | 9811956 |
| 8449. | BE645241 | EST | 9969552 |
| 8450. | BE669441 | EST | 10029982 |
| 8451. | BE739783 | EST | 10153775 |
| 8452. | BE746368 | EST | 10160360 |
| 8453. | BE833262 | EST | 10265640 |
| 8454. | BE843975 | EST | 10276353 |
| 8455. | BE882091 | EST | 10330867 |
| 8456. | F01237 | EST | 708316 |
| 8457. | F06900 | EST | 672518 |
| 8458. | F17743 | EST | 1134010 |
| 8459. | F18959 | EST | 4825376 |
| 8460. | F22763 | EST | 2061939 |
| 8461. | H05231 | EST | 868783 |
| 8462. | H42133 | EST | 918185 |
| 8463. | H61787 | EST | 1014619 |
| 8464. | H73375 | EST | 1047625 |
| 8465. | H82025 | EST | 1060114 |
| 8466. | H83590 | EST | 1062261 |
| 8467. | H88681 | EST | 1070941 |
| 8468. | L44281 | EST | 1048741 |
| 8469. | M85929 | EST | 274581 |
| 8470. | N21213 | EST | 1126383 |
| 8471. | N25157 | EST | 1139307 |
| 8472. | N26844 | EST | 1141192 |
| 8473. | N57978 | EST | 1201868 |
| 8474. | N58953 | EST | 1202843 |
| 8475. | N62799 | EST | 1210628 |
| 8476. | N74353 | EST | 1231638 |
| 8477. | N79666 | EST | 1242367 |
| 8478. | N98328 | EST | 1269973 |
| 8479. | R07181 | EST | 759104 |
| 8480. | R10004 | EST | 761960 |
| 8481. | R48898 | EST | 810924 |
| 8482. | R62742 | EST | 834621 |
| 8483. | T17239 | EST | 519401 |
| 8484. | T50506 | EST | 652366 |
| 8485. | T52858 | EST | 654718 |
| 8486. | T66102 | EST | 675147 |
| 8487. | U54711 | EST | 1381131 |
| 8488. | W02248 | EST | 1274227 |
| 8489. | W51959 | EST | 1349213 |
| 8490. | W60504 | EST | 1367331 |
| 8491. | W63634 | EST | 1371215 |
| 8492. | W72551 | EST | 1382238 |
| 8493. | W80646 | EST | 1391663 |
| 8494. | W87562 | EST | 1401617 |
| 8495. | A09447 | NUCPATENT | 490550 |
| 8496. | A16010 | NUCPATENT | 492019 |
| 8497. | A26283 | NUCPATENT | 1247686 |
| 8498. | A26358 | NUCPATENT | 904919 |
| 8499. | A26385 | NUCPATENT | 904942 |
| 8500. | A27054 | NUCPATENT | N/A |
| 8501. | A37037 | NUCPATENT | N/A |
| 8502. | A43567 | NUCPATENT | 2298754 |
| 8503. | T18059 | NUCPATENT | 462845 |
| 8504. | T59274 | NUCPATENT | 661111 |
| 8505. | T88982 | NUCPATENT | 717495 |
| 8506. | T91302 | NUCPATENT | 723215 |
| 8507. | V46313 | NUCPATENT | N/A |
| 8508. | X02994 | NUCPATENT | 28379 |
| 8509. | X27245 | NUCPATENT | N/A |
| 8510. | X37458 | NUCPATENT | N/A |
| 8511. | X39469 | NUCPATENT | N/A |
| 8512. | X61371 | NUCPATENT | N/A |
| 8513. | Z42097 | NUCPATENT | 564336 |
| 8514. | Z42848 | NUCPATENT | 570421 |
| 8515. | AC31081 | PREPATNUC | N/A |
| 8516. | AC41996 | PREPATNUC | N/A |
| 8517. | AC46032 | PREPATNUC | N/A |
| 8518. | AC46455 | PREPATNUC | N/A |
| 8519. | A06800 | Genbank | 490045 |
| 8520. | A08691 | Genbank | 412179 |
| 8521. | A14571 | Genbank | 490133 |
| 8522. | A16794 | Genbank | 512417 |
| 8523. | A17546 | Genbank | 490012 |
| 8524. | A21185 | Genbank | 579591 |
| 8525. | A23013 | Genbank | 825583 |
| 8526. | A29119 | Genbank | 1247520 |
| 8527. | A30438 | Genbank | 1567031 |
| 8528. | A31920 | Genbank | 1247569 |
| 8529. | A36460 | Genbank | 2293778 |
| 8530. | A36461 | Genbank | 2293779 |
| 8531. | A49472 | Genbank | 2302930 |
| 8532. | A61385 | Genbank | 3715797 |
| 8533. | A69527 | Genbank | 4774176 |
| 8534. | AB000887 | Genbank | 2189952 |
| 8535. | AB001106 | Genbank | 1850083 |
| 8536. | AB001993 | Genbank | 3046868 |
| 8537. | AB002332 | Genbank | 2224608 |
| 8538. | AB002370 | Genbank | 2280483 |
| 8539. | AB002377 | Genbank | 6634024 |
| 8540. | AB002390 | Genbank | 2280487 |
| 8541. | AB002409 | Genbank | 2335034 |
| 8542. | AB002439 | Genbank | 2943814 |
| 8543. | AB003476 | Genbank | 2081606 |
| 8544. | AB003730 | Genbank | 2627128 |
| 8545. | AB004066 | Genbank | 2308996 |
| 8546. | AB004303 | Genbank | 4176415 |
| 8547. | AB004546 | Genbank | 3308972 |
| 8548. | AB005293 | Genbank | 3041770 |
| 8549. | AB005791 | Genbank | 2789439 |
| 8550. | AB006534 | Genbank | 2924619 |
| 8551. | AB006621 | Genbank | 2564313 |
| 8552. | AB007619 | Genbank | 2465179 |
| 8553. | AB007858 | Genbank | 2662076 |
| 8554. | AB007865 | Genbank | 2662090 |
| 8555. | AB007869 | Genbank | 2662098 |
| 8556. | AB007888 | Genbank | 2887430 |
| 8557. | AB007916 | Genbank | 6683704 |
| 8558. | AB007927 | Genbank | 3413877 |
| 8559. | AB007940 | Genbank | 3413903 |
| 8560. | AB007956 | Genbank | 3413930 |
| 8561. | AB007960 | Genbank | 3413934 |
| 8562. | AB008109 | Genbank | 2554613 |
| 8563. | AB009010 | Genbank | 2647407 |
| 8564. | AB011089 | Genbank | 3043557 |
| 8565. | AB011100 | Genbank | 6683714 |
| 8566. | AB011102 | Genbank | 3043583 |
| 8567. | AB011108 | Genbank | 3043595 |
| 8568. | AB011141 | Genbank | 3043661 |
| 8569. | AB011159 | Genbank | 3043697 |
| 8570. | AB011173 | Genbank | 3043725 |
| 8571. | AB013139 | Genbank | 3169124 |
| 8572. | AB014080 | Genbank | 5672592 |
| 8573. | AB014458 | Genbank | 3928761 |
| 8574. | AB014486 | Genbank | 4062959 |
| 8575. | AB014536 | Genbank | 3327085 |
| 8576. | AB014547 | Genbank | 3327107 |
| 8577. | AB014560 | Genbank | 3327133 |
| 8578. | AB014597 | Genbank | 3327207 |
| 8579. | AB015333 | Genbank | 3970855 |
| 8580. | AB015639 | Genbank | 5821139 |
| 8581. | AB015907 | Genbank | 4218063 |
| 8582. | AB015982 | Genbank | 5926628 |
| 8583. | AB016247 | Genbank | 3721881 |
| 8584. | AB017494 | Genbank | 4589719 |
| 8585. | AB017644 | Genbank | 4586929 |
| 8586. | AB017708 | Genbank | 3650434 |
| 8587. | AB018257 | Genbank | 3882148 |
| 8588. | AB018272 | Genbank | 3882178 |
| 8589. | AB018327 | Genbank | 3882288 |
| 8590. | AB018329 | Genbank | 3882292 |
| 8591. | AB018330 | Genbank | 3882294 |
| 8592. | AB018566 | Genbank | 4126977 |
| 8593. | AB019437 | Genbank | 4512267 |
| 8594. | AB019438 | Genbank | 4512277 |
| 8595. | AB019439 | Genbank | 4512287 |
| 8596. | AB019440 | Genbank | 4512300 |
| 8597. | AB019568 | Genbank | 3885371 |
| 8598. | AB019569 | Genbank | 3885372 |
| 8599. | AB020532 | Genbank | 6497098 |
| 8600. | AB020629 | Genbank | 4240129 |
| 8601. | AB020634 | Genbank | 4240142 |
| 8602. | AB020641 | Genbank | 4240156 |
| 8603. | AB020658 | Genbank | 4240190 |
| 8604. | AB020680 | Genbank | 4240234 |
| 8605. | AB020692 | Genbank | 4240258 |
| 8606. | AB020700 | Genbank | 4240274 |
| 8607. | AB020703 | Genbank | 4240280 |
| 8608. | AB020721 | Genbank | 4240316 |
| 8609. | AB020863 | Genbank | 4003383 |
| 8610. | AB021288 | Genbank | 4038732 |
| 8611. | AB022192 | Genbank | 6682846 |
| 8612. | AB022655 | Genbank | 5360676 |
| 8613. | AB022663 | Genbank | 5019617 |
| 8614. | AB023051 | Genbank | 5672606 |
| 8615. | AB023209 | Genbank | 4589627 |
| 8616. | AB023210 | Genbank | 4589629 |
| 8617. | AB023230 | Genbank | 4589675 |
| 8618. | AB023234 | Genbank | 4589683 |
| 8619. | AB023652 | Genbank | 4630770 |
| 8620. | AB024518 | Genbank | 4520327 |
| 8621. | AB024703 | Genbank | 5931544 |
| 8622. | AB026898 | Genbank | 4835382 |
| 8623. | AB027258 | Genbank | 6429662 |
| 8624. | AB028639 | Genbank | 5102943 |
| 8625. | AB028893 | Genbank | 6552364 |
| 8626. | AB028945 | Genbank | 5689380 |
| 8627. | AB028961 | Genbank | 5689412 |
| 8628. | AB028963 | Genbank | 5689416 |
| 8629. | AB028973 | Genbank | 5689436 |
| 8630. | AB028981 | Genbank | 5689452 |
| 8631. | AB029032 | Genbank | 5689554 |
| 8632. | AB029036 | Genbank | 5689562 |
| 8633. | AB029309 | Genbank | 6594305 |
| 8634. | AB032948 | Genbank | 6329727 |
| 8635. | AB032969 | Genbank | 6329965 |
| 8636. | AB032971 | Genbank | 6330018 |
| 8637. | AB032973 | Genbank | 6330032 |
| 8638. | AB032983 | Genbank | 6330128 |
| 8639. | AB033034 | Genbank | 6382021 |
| 8640. | AB033049 | Genbank | 6330616 |
| 8641. | AB033051 | Genbank | 6330667 |
| 8642. | AB033064 | Genbank | 6330771 |
| 8643. | AB033071 | Genbank | 6330825 |
| 8644. | AB033079 | Genbank | 6382025 |
| 8645. | AB034205 | Genbank | 6899845 |
| 8646. | AB037730 | Genbank | 7242972 |
| 8647. | AB037763 | Genbank | 7243064 |
| 8648. | AB037765 | Genbank | 7243068 |
| 8649. | AB037788 | Genbank | 7243114 |
| 8650. | AB037790 | Genbank | 7243118 |
| 8651. | AB037795 | Genbank | 7243128 |
| 8652. | AB037801 | Genbank | 7243140 |
| 8653. | AB037803 | Genbank | 7243144 |
| 8654. | AB037835 | Genbank | 7243208 |
| 8655. | AB037841 | Genbank | 7243220 |
| 8656. | AB037842 | Genbank | 7243222 |
| 8657. | AB037849 | Genbank | 7243236 |
| 8658. | AB037851 | Genbank | 7243240 |
| 8659. | AB037856 | Genbank | 7243267 |
| 8660. | AB039667 | Genbank | 7209865 |
| 8661. | AB040927 | Genbank | 7959248 |
| 8662. | AC000015 | Genbank | 4263638 |
| 8663. | AC000064 | Genbank | 1669369 |
| 8664. | AC000394 | Genbank | 2133911 |
| 8665. | AC000403 | Genbank | 2133864 |
| 8666. | AC002038 | Genbank | 2226439 |
| 8667. | AC002064 | Genbank | 2076723 |
| 8668. | AC002076 | Genbank | 2078461 |
| 8669. | AC002451 | Genbank | 2337882 |
| 8670. | AC002480 | Genbank | 2340098 |
| 8671. | AC002528 | Genbank | 2388554 |
| 8672. | AC002546 | Genbank | 2583101 |
| 8673. | AC003016 | Genbank | 2547254 |
| 8674. | AC003075 | Genbank | 2588637 |
| 8675. | AC003108 | Genbank | 2833632 |
| 8676. | AC003682 | Genbank | 3264845 |
| 8677. | AC003692 | Genbank | 2731601 |
| 8678. | AC003958 | Genbank | 2828773 |
| 8679. | AC003976 | Genbank | 2828772 |
| 8680. | AC003985 | Genbank | 2769692 |
| 8681. | AC004022 | Genbank | 3598727 |
| 8682. | AC004032 | Genbank | 5032329 |
| 8683. | AC004053 | Genbank | 3687218 |
| 8684. | AC004098 | Genbank | 3097872 |
| 8685. | AC004148 | Genbank | 3482960 |
| 8686. | AC004230 | Genbank | 4028938 |
| 8687. | AC004236 | Genbank | 2914668 |
| 8688. | AC004448 | Genbank | 11128431 |
| 8689. | AC004452 | Genbank | 2979602 |
| 8690. | AC004477 | Genbank | 3688107 |
| 8691. | AC004526 | Genbank | 3779035 |
| 8692. | AC004527 | Genbank | 4760422 |
| 8693. | AC004584 | Genbank | 3417305 |
| 8694. | AC004603 | Genbank | 3077822 |
| 8695. | AC004605 | Genbank | 3337395 |
| 8696. | AC004615 | Genbank | 3080660 |
| 8697. | AC004686 | Genbank | 3688105 |
| 8698. | AC004837 | Genbank | 3845417 |
| 8699. | AC004850 | Genbank | 4309890 |
| 8700. | AC004854 | Genbank | 4827328 |
| 8701. | AC004900 | Genbank | 5757547 |
| 8702. | AC004902 | Genbank | 5757546 |
| 8703. | AC004940 | Genbank | 3953511 |
| 8704. | AC004955 | Genbank | 4753267 |
| 8705. | AC005027 | Genbank | 4753250 |
| 8706. | AC005045 | Genbank | 4508117 |
| 8707. | AC005062 | Genbank | 4150931 |
| 8708. | AC005082 | Genbank | 5836159 |
| 8709. | AC005156 | Genbank | 3242759 |
| 8710. | AC005206 | Genbank | 3319117 |
| 8711. | AC005324 | Genbank | 3366582 |
| 8712. | AC005385 | Genbank | 6249680 |
| 8713. | AC005392 | Genbank | 3399669 |
| 8714. | AC005409 | Genbank | 4249432 |
| 8715. | AC005479 | Genbank | 4508155 |
| 8716. | AC005509 | Genbank | 4176355 |
| 8717. | AC005544 | Genbank | 3650045 |
| 8718. | AC005603 | Genbank | 3522968 |
| 8719. | AC005612 | Genbank | 3540153 |
| 8720. | AC005740 | Genbank | 3687210 |
| 8721. | AC005799 | Genbank | 3789715 |
| 8722. | AC005837 | Genbank | 3849820 |
| 8723. | AC005877 | Genbank | 6249671 |
| 8724. | AC005899 | Genbank | 3935221 |
| 8725. | AC005912 | Genbank | 4165005 |
| 8726. | AC005922 | Genbank | 3873182 |
| 8727. | AC006022 | Genbank | 4153867 |
| 8728. | AC006033 | Genbank | 4309948 |
| 8729. | AC006050 | Genbank | 4079627 |
| 8730. | AC006064 | Genbank | 4572650 |
| 8731. | AC006077 | Genbank | 3935193 |
| 8732. | AC006084 | Genbank | 3953485 |
| 8733. | AC006159 | Genbank | 7243950 |
| 8734. | AC006165 | Genbank | 3980464 |
| 8735. | AC006196 | Genbank | 6001976 |
| 8736. | AC006252 | Genbank | 4309923 |
| 8737. | AC006344 | Genbank | 4508150 |
| 8738. | AC006352 | Genbank | 4753268 |
| 8739. | AC006480 | Genbank | 6289252 |
| 8740. | AC006487 | Genbank | 11128438 |
| 8741. | AC006512 | Genbank | 4926863 |
| 8742. | AC006530 | Genbank | 4680764 |
| 8743. | AC006559 | Genbank | 4887253 |
| 8744. | AC006581 | Genbank | 4914350 |
| 8745. | AC006971 | Genbank | 4731074 |
| 8746. | AC007000 | Genbank | 7243924 |
| 8747. | AC007041 | Genbank | 5708471 |
| 8748. | AC007055 | Genbank | 4885691 |
| 8749. | AC007115 | Genbank | 4895146 |
| 8750. | AC007130 | Genbank | 5001538 |
| 8751. | AC007199 | Genbank | 4558768 |
| 8752. | AC007204 | Genbank | 4559317 |
| 8753. | AC007314 | Genbank | 4662678 |
| 8754. | AC007401 | Genbank | 10440760 |
| 8755. | AC007537 | Genbank | 4914348 |
| 8756. | AC007563 | Genbank | 6289217 |
| 8757. | AC007564 | Genbank | 4982534 |
| 8758. | AC007655 | Genbank | 4895153 |
| 8759. | AC007690 | Genbank | 5815494 |
| 8760. | AC007750 | Genbank | 6094634 |
| 8761. | AC007966 | Genbank | 5836190 |
| 8762. | AC008008 | Genbank | 5656683 |
| 8763. | AC008012 | Genbank | 5762548 |
| 8764. | AC008040 | Genbank | 5922025 |
| 8765. | AC008115 | Genbank | 5801654 |
| 8766. | AC008123 | Genbank | 6006046 |
| 8767. | AC008126 | Genbank | 5923639 |
| 8768. | AC008417 | Genbank | 6730695 |
| 8769. | AC008572 | Genbank | 7341444 |
| 8770. | AC008865 | Genbank | 7019284 |
| 8771. | AC009223 | Genbank | 6042098 |
| 8772. | AC009403 | Genbank | 10440729 |
| 8773. | AC010197 | Genbank | 6143841 |
| 8774. | AC010202 | Genbank | 6598666 |
| 8775. | AC010386 | Genbank | 7105518 |
| 8776. | AC010494 | Genbank | 7109400 |
| 8777. | AC010517 | Genbank | 6223625 |
| 8778. | AC011088 | Genbank | 7960348 |
| 8779. | AC011362 | Genbank | 6094545 |
| 8780. | AC012087 | Genbank | 7114465 |
| 8781. | AC012262 | Genbank | 7381642 |
| 8782. | AC016138 | Genbank | 7021552 |
| 8783. | AC020949 | Genbank | 7211911 |
| 8784. | AC021092 | Genbank | 6693370 |
| 8785. | AF000231 | Genbank | 2149974 |
| 8786. | AF000381 | Genbank | 2565195 |
| 8787. | AF000547 | Genbank | 2232070 |
| 8788. | AF000652 | Genbank | 2795862 |
| 8789. | AF000982 | Genbank | 2580549 |
| 8790. | AF000992 | Genbank | 2580569 |
| 8791. | AF001862 | Genbank | 2232149 |
| 8792. | AF001902 | Genbank | 2078326 |
| 8793. | AF002668 | Genbank | 2232173 |
| 8794. | AF004327 | Genbank | 2257932 |
| 8795. | AF004561 | Genbank | 2209346 |
| 8796. | AF005422 | Genbank | 2209234 |
| 8797. | AF006010 | Genbank | 4101694 |
| 8798. | AF006086 | Genbank | 2282037 |
| 8799. | AF006636 | Genbank | 2198854 |
| 8800. | AF007544 | Genbank | 2970122 |
| 8801. | AF007791 | Genbank | 3779196 |
| 8802. | AF008442 | Genbank | 2266928 |
| 8803. | AF009202 | Genbank | 2454507 |
| 8804. | AF011793 | Genbank | 2352903 |
| 8805. | AF012073 | Genbank | 2735856 |
| 8806. | AF013622 | Genbank | 3135406 |
| 8807. | AF013759 | Genbank | 3153208 |
| 8808. | AF014882 | Genbank | 2582056 |
| 8809. | AF015283 | Genbank | 2384720 |
| 8810. | AF015767 | Genbank | 2353176 |
| 8811. | AF015812 | Genbank | 2599359 |
| 8812. | AF017104 | Genbank | 2736085 |
| 8813. | AF017269 | Genbank | 2394305 |
| 8814. | AF017445 | Genbank | 2582184 |
| 8815. | AF017782 | Genbank | 3986404 |
| 8816. | AF019369 | Genbank | 2623562 |
| 8817. | AF023266 | Genbank | 4103447 |
| 8818. | AF024714 | Genbank | 2558941 |
| 8819. | AF025772 | Genbank | 5757624 |
| 8820. | AF026169 | Genbank | 5091668 |
| 8821. | AF026291 | Genbank | 2559007 |
| 8822. | AF026445 | Genbank | 2739086 |
| 8823. | AF027153 | Genbank | 2739093 |
| 8824. | AF027159 | Genbank | 2623586 |
| 8825. | AF027205 | Genbank | 2598967 |
| 8826. | AF027964 | Genbank | 2695662 |
| 8827. | AF028832 | Genbank | 3287488 |
| 8828. | AF030339 | Genbank | 3176761 |
| 8829. | AF030409 | Genbank | 2944232 |
| 8830. | AF031379 | Genbank | 4894208 |
| 8831. | AF033095 | Genbank | 2645728 |
| 8832. | AF034799 | Genbank | 3309532 |
| 8833. | AF035286 | Genbank | 2661038 |
| 8834. | AF035289 | Genbank | 2661043 |
| 8835. | AF035810 | Genbank | 2674167 |
| 8836. | AF035940 | Genbank | 2909829 |
| 8837. | AF038202 | Genbank | 2795923 |
| 8838. | AF038451 | Genbank | 3779225 |
| 8839. | AF038960 | Genbank | 3329389 |
| 8840. | AF039656 | Genbank | 2773159 |
| 8841. | AF039687 | Genbank | 3170173 |
| 8842. | AF039907 | Genbank | 2754858 |
| 8843. | AF041259 | Genbank | 3335396 |
| 8844. | AF043906 | Genbank | 2832292 |
| 8845. | AF045184 | Genbank | 3417598 |
| 8846. | AF047020 | Genbank | 4204096 |
| 8847. | AF047439 | Genbank | 3335133 |
| 8848. | AF047448 | Genbank | 2961148 |
| 8849. | AF047826 | Genbank | 2935422 |
| 8850. | AF049910 | Genbank | 3435156 |
| 8851. | AF050163 | Genbank | 3293304 |
| 8852. | AF051160 | Genbank | 2961198 |
| 8853. | AF051684 | Genbank | 3047259 |
| 8854. | AF051941 | Genbank | 3893858 |
| 8855. | AF052097 | Genbank | 3360404 |
| 8856. | AF052115 | Genbank | 3360422 |
| 8857. | AF052146 | Genbank | 3360455 |
| 8858. | AF052155 | Genbank | 3360466 |
| 8859. | AF052179 | Genbank | 3360490 |
| 8860. | AF052578 | Genbank | 2967847 |
| 8861. | AF054174 | Genbank | 3341991 |
| 8862. | AF054181 | Genbank | 3342003 |
| 8863. | AF054284 | Genbank | 4033734 |
| 8864. | AF054988 | Genbank | 3005699 |
| 8865. | AF054990 | Genbank | 3005703 |
| 8866. | AF054999 | Genbank | 3005714 |
| 8867. | AF056332 | Genbank | 3044135 |
| 8868. | AF057160 | Genbank | 3694919 |
| 8869. | AF057356 | Genbank | 3063652 |
| 8870. | AF058953 | Genbank | 3766196 |
| 8871. | AF061016 | Genbank | 3127126 |
| 8872. | AF061261 | Genbank | 3779239 |
| 8873. | AF062072 | Genbank | 3668065 |
| 8874. | AF062149 | Genbank | 3170760 |
| 8875. | AF062232 | Genbank | 3170930 |
| 8876. | AF062233 | Genbank | 3170932 |
| 8877. | AF062249 | Genbank | 3170964 |
| 8878. | AF063605 | Genbank | 4071360 |
| 8879. | AF063611 | Genbank | 4731856 |
| 8880. | AF064801 | Genbank | 3395786 |
| 8881. | AF064824 | Genbank | 3290171 |
| 8882. | AF064879 | Genbank | 4321848 |
| 8883. | AF065391 | Genbank | 4191326 |
| 8884. | AF065481 | Genbank | 4867995 |
| 8885. | AF065482 | Genbank | 3152937 |
| 8886. | AF065483 | Genbank | 3152939 |
| 8887. | AF065485 | Genbank | 3873215 |
| 8888. | AF067420 | Genbank | 3201899 |
| 8889. | AF069301 | Genbank | 3193335 |
| 8890. | AF069307 | Genbank | 4884549 |
| 8891. | AF069313 | Genbank | 7145105 |
| 8892. | AF069601 | Genbank | 7239697 |
| 8893. | AF070418 | Genbank | 3236451 |
| 8894. | AF070523 | Genbank | 3764088 |
| 8895. | AF070525 | Genbank | 3387880 |
| 8896. | AF070542 | Genbank | 3387901 |
| 8897. | AF070555 | Genbank | 3387920 |
| 8898. | AF070556 | Genbank | 3387921 |
| 8899. | AF070561 | Genbank | 3387928 |
| 8900. | AF070635 | Genbank | 3283905 |
| 8901. | AF070640 | Genbank | 3283913 |
| 8902. | AF070646 | Genbank | 3283920 |
| 8903. | AF070649 | Genbank | 3283923 |
| 8904. | AF070652 | Genbank | 4454679 |
| 8905. | AF070656 | Genbank | 4454687 |
| 8906. | AF070667 | Genbank | 4454709 |
| 8907. | AF070668 | Genbank | 4454711 |
| 8908. | AF073298 | Genbank | 3641537 |
| 8909. | AF073344 | Genbank | 5410229 |
| 8910. | AF075587 | Genbank | 3319325 |
| 8911. | AF077030 | Genbank | 4689107 |
| 8912. | AF077037 | Genbank | 4689121 |
| 8913. | AF077046 | Genbank | 4689139 |
| 8914. | AF077050 | Genbank | 4689147 |
| 8915. | AF077053 | Genbank | 4689153 |
| 8916. | AF077200 | Genbank | 4679013 |
| 8917. | AF077202 | Genbank | 4679017 |
| 8918. | AF077205 | Genbank | 4679023 |
| 8919. | AF077207 | Genbank | 4679027 |
| 8920. | AF077515 | Genbank | 3832694 |
| 8921. | AF077951 | Genbank | 3643106 |
| 8922. | AF078845 | Genbank | 5531804 |
| 8923. | AF078850 | Genbank | 5531814 |
| 8924. | AF080092 | Genbank | 4322311 |
| 8925. | AF081195 | Genbank | 3928854 |
| 8926. | AF081484 | Genbank | 3420928 |
| 8927. | AF082569 | Genbank | 4206702 |
| 8928. | AF083190 | Genbank | 3599414 |
| 8929. | AF083384 | Genbank | 3746839 |
| 8930. | AF083395 | Genbank | 4106817 |
| 8931. | AF083441 | Genbank | 5813822 |
| 8932. | AF083930 | Genbank | 4416182 |
| 8933. | AF084520 | Genbank | 5052120 |
| 8934. | AF084555 | Genbank | 5813858 |
| 8935. | AF085359 | Genbank | 5114052 |
| 8936. | AF085481 | Genbank | 4336415 |
| 8937. | AF086002 | Genbank | 3483347 |
| 8938. | AF086175 | Genbank | 3483520 |
| 8939. | AF086214 | Genbank | 3483559 |
| 8940. | AF088867 | Genbank | 6652811 |
| 8941. | AF090095 | Genbank | 4063630 |
| 8942. | AF090904 | Genbank | 6690184 |
| 8943. | AF090928 | Genbank | 6690222 |
| 8944. | AF091076 | Genbank | 3859989 |
| 8945. | AF091263 | Genbank | 4140646 |
| 8946. | AF091622 | Genbank | 6648927 |
| 8947. | AF091871 | Genbank | 4262371 |
| 8948. | AF092128 | Genbank | 5138905 |
| 8949. | AF092135 | Genbank | 5138919 |
| 8950. | AF095891 | Genbank | 4185494 |
| 8951. | AF097362 | Genbank | 6165617 |
| 8952. | AF097514 | Genbank | 4808600 |
| 8953. | AF098807 | Genbank | 4877581 |
| 8954. | AF099935 | Genbank | 3860092 |
| 8955. | AF100141 | Genbank | 3978516 |
| 8956. | AF100741 | Genbank | 5138992 |
| 8957. | AF103768 | Genbank | 6179855 |
| 8958. | AF104238 | Genbank | 5880486 |
| 8959. | AF104914 | Genbank | 4206125 |
| 8960. | AF104921 | Genbank | 9409793 |
| 8961. | AF105421 | Genbank | 4838128 |
| 8962. | AF106019 | Genbank | 5359630 |
| 8963. | AF106681 | Genbank | 5410327 |
| 8964. | AF106862 | Genbank | 5757883 |
| 8965. | AF106966 | Genbank | 4008088 |
| 8966. | AF110368 | Genbank | 6502538 |
| 8967. | AF110460 | Genbank | 4324698 |
| 8968. | AF110774 | Genbank | 6523794 |
| 8969. | AF110824 | Genbank | 4927639 |
| 8970. | AF112204 | Genbank | 6563195 |
| 8971. | AF113015 | Genbank | 6642753 |
| 8972. | AF113016 | Genbank | 6642755 |
| 8973. | AF113686 | Genbank | 6855614 |
| 8974. | AF113700 | Genbank | 6855634 |
| 8975. | AF113702 | Genbank | 6855636 |
| 8976. | AF115384 | Genbank | 4566745 |
| 8977. | AF115402 | Genbank | 4559272 |
| 8978. | AF116347 | Genbank | 6650721 |
| 8979. | AF118091 | Genbank | 6650827 |
| 8980. | AF118808 | Genbank | 11131781 |
| 8981. | AF123462 | Genbank | 4240565 |
| 8982. | AF125097 | Genbank | 5106989 |
| 8983. | AF125158 | Genbank | 4455117 |
| 8984. | AF126181 | Genbank | 4732088 |
| 8985. | AF126736 | Genbank | 4454564 |
| 8986. | AF129537 | Genbank | 6164623 |
| 8987. | AF130366 | Genbank | 4530576 |
| 8988. | AF131738 | Genbank | 4406548 |
| 8989. | AF131802 | Genbank | 4406633 |
| 8990. | AF131817 | Genbank | 4406652 |
| 8991. | AF131838 | Genbank | 4406677 |
| 8992. | AF131857 | Genbank | 4406704 |
| 8993. | AF132000 | Genbank | 4530586 |
| 8994. | AF132047 | Genbank | 4838516 |
| 8995. | AF132940 | Genbank | 4680650 |
| 8996. | AF132947 | Genbank | 4680664 |
| 8997. | AF135162 | Genbank | 7259481 |
| 8998. | AF135488 | Genbank | 5668619 |
| 8999. | AF138300 | Genbank | 5532410 |
| 9000. | AF138302 | Genbank | 5532412 |
| 9001. | AF141347 | Genbank | 4929133 |
| 9002. | AF143889 | Genbank | 4895034 |
| 9003. | AF144700 | Genbank | 5107075 |
| 9004. | AF145385 | Genbank | 4929329 |
| 9005. | AF146651 | Genbank | 5020073 |
| 9006. | AF147331 | Genbank | 4761682 |
| 9007. | AF147334 | Genbank | 4761685 |
| 9008. | AF147367 | Genbank | 4761718 |
| 9009. | AF151028 | Genbank | 7106777 |
| 9010. | AF151050 | Genbank | 7106821 |
| 9011. | AF151538 | Genbank | 6288762 |
| 9012. | AF151803 | Genbank | 4929558 |
| 9013. | AF151826 | Genbank | 4929604 |
| 9014. | AF151872 | Genbank | 4929696 |
| 9015. | AF151878 | Genbank | 4929708 |
| 9016. | AF151885 | Genbank | 4929722 |
| 9017. | AF152961 | Genbank | 5499740 |
| 9018. | AF153821 | Genbank | 5002378 |
| 9019. | AF156165 | Genbank | 7145093 |
| 9020. | AF156965 | Genbank | 5731112 |
| 9021. | AF161384 | Genbank | 6841181 |
| 9022. | AF161458 | Genbank | 6841439 |
| 9023. | AF161466 | Genbank | 6841455 |
| 9024. | AF161473 | Genbank | 6841469 |
| 9025. | AF161501 | Genbank | 6841525 |
| 9026. | AF161522 | Genbank | 6841567 |
| 9027. | AF161530 | Genbank | 6841583 |
| 9028. | AF161541 | Genbank | 6841349 |
| 9029. | AF161547 | Genbank | 6841361 |
| 9030. | AF161554 | Genbank | 6841375 |
| 9031. | AF163254 | Genbank | 5733599 |
| 9032. | AF165124 | Genbank | 5738137 |
| 9033. | AF172093 | Genbank | 7248635 |
| 9034. | AF173856 | Genbank | 7021320 |
| 9035. | AF174113 | Genbank | 5834185 |
| 9036. | AF174496 | Genbank | 7108914 |
| 9037. | AF174591 | Genbank | 6164724 |
| 9038. | AF176574 | Genbank | 5762481 |
| 9039. | AF176700 | Genbank | 6103638 |
| 9040. | AF177765 | Genbank | 6175872 |
| 9041. | AF180322 | Genbank | 5881245 |
| 9042. | AF180473 | Genbank | 6856202 |
| 9043. | AF182844 | Genbank | 6003571 |
| 9044. | AF184764 | Genbank | 6110571 |
| 9045. | AF187554 | Genbank | 6653225 |
| 9046. | AF188611 | Genbank | 6470149 |
| 9047. | AF191298 | Genbank | 7656642 |
| 9048. | AF191339 | Genbank | 6180012 |
| 9049. | AF196969 | Genbank | 6289073 |
| 9050. | AF201077 | Genbank | 6456748 |
| 9051. | AF201947 | Genbank | 6563291 |
| 9052. | AF201951 | Genbank | 6563299 |
| 9053. | AF203815 | Genbank | 6979641 |
| 9054. | AF205588 | Genbank | 6531675 |
| 9055. | AF207550 | Genbank | 6470333 |
| 9056. | AF213969 | Genbank | 6626178 |
| 9057. | AF224669 | Genbank | 7012905 |
| 9058. | AF226614 | Genbank | 7109248 |
| 9059. | AF241728 | Genbank | 7263192 |
| 9060. | AJ000881 | Genbank | 2924308 |
| 9061. | AJ001306 | Genbank | 2370148 |
| 9062. | AJ001309 | Genbank | 3171907 |
| 9063. | AJ002308 | Genbank | 2959871 |
| 9064. | AJ002675 | Genbank | 4007582 |
| 9065. | AJ010442 | Genbank | 3954884 |
| 9066. | AJ010842 | Genbank | 3646129 |
| 9067. | AJ130733 | Genbank | 4995298 |
| 9068. | AJ131244 | Genbank | 3947687 |
| 9069. | AJ131245 | Genbank | 3947689 |
| 9070. | AJ132637 | Genbank | 5689741 |
| 9071. | AJ223812 | Genbank | 2894518 |
| 9072. | AJ224162 | Genbank | 2897594 |
| 9073. | AJ224442 | Genbank | 2911586 |
| 9074. | AJ225773 | Genbank | 2808994 |
| 9075. | AJ239341 | Genbank | 4456503 |
| 9076. | AJ242975 | Genbank | 5101765 |
| 9077. | AJ243207 | Genbank | 5263023 |
| 9078. | AJ243663 | Genbank | 6688144 |
| 9079. | AJ244946 | Genbank | 4995351 |
| 9080. | AJ245004 | Genbank | 4995466 |
| 9081. | AJ250915 | Genbank | 6996445 |
| 9082. | AJ272197 | Genbank | 7242619 |
| 9083. | AJ387747 | Genbank | 6562532 |
| 9084. | AK000049 | Genbank | 7019880 |
| 9085. | AK000087 | Genbank | 7019946 |
| 9086. | AK000093 | Genbank | 7019956 |
| 9087. | AK000191 | Genbank | 7020112 |
| 9088. | AK000231 | Genbank | 7020177 |
| 9089. | AK000260 | Genbank | 7020221 |
| 9090. | AK000290 | Genbank | 7020272 |
| 9091. | AK000299 | Genbank | 7020288 |
| 9092. | AK000414 | Genbank | 7020486 |
| 9093. | AK000462 | Genbank | 7020566 |
| 9094. | AK000509 | Genbank | 7020648 |
| 9095. | AK000544 | Genbank | 7020710 |
| 9096. | AK000548 | Genbank | 7020717 |
| 9097. | AK000567 | Genbank | 7020750 |
| 9098. | AK000587 | Genbank | 7020782 |
| 9099. | AK000624 | Genbank | 7020839 |
| 9100. | AK000635 | Genbank | 7020858 |
| 9101. | AK000639 | Genbank | 7020863 |
| 9102. | AK000648 | Genbank | 7020877 |
| 9103. | AK000669 | Genbank | 7020909 |
| 9104. | AK000680 | Genbank | 7020924 |
| 9105. | AK000688 | Genbank | 7020934 |
| 9106. | AK000713 | Genbank | 7020973 |
| 9107. | AK000714 | Genbank | 7020975 |
| 9108. | AK000724 | Genbank | 7020989 |
| 9109. | AK000790 | Genbank | 7021092 |
| 9110. | AK000946 | Genbank | 7021928 |
| 9111. | AK000953 | Genbank | 7021938 |
| 9112. | AK001043 | Genbank | 7022068 |
| 9113. | AK001050 | Genbank | 7022077 |
| 9114. | AK001067 | Genbank | 7022104 |
| 9115. | AK001112 | Genbank | 7022170 |
| 9116. | AK001224 | Genbank | 7022345 |
| 9117. | AK001311 | Genbank | 7022486 |
| 9118. | AK001313 | Genbank | 7022490 |
| 9119. | AK001319 | Genbank | 7022500 |
| 9120. | AK001364 | Genbank | 7022577 |
| 9121. | AK001478 | Genbank | 7022760 |
| 9122. | AK001486 | Genbank | 7022771 |
| 9123. | AK001488 | Genbank | 7022775 |
| 9124. | AK001521 | Genbank | 7022828 |
| 9125. | AK001528 | Genbank | 7022839 |
| 9126. | AK001536 | Genbank | 7022851 |
| 9127. | AK001718 | Genbank | 7023153 |
| 9128. | AK001721 | Genbank | 7023158 |
| 9129. | AK001775 | Genbank | 7023255 |
| 9130. | AK001801 | Genbank | 7023300 |
| 9131. | AK001886 | Genbank | 7023430 |
| 9132. | AK001915 | Genbank | 7023475 |
| 9133. | AK001926 | Genbank | 7023492 |
| 9134. | AK001930 | Genbank | 7023500 |
| 9135. | AK001934 | Genbank | 7023506 |
| 9136. | AK001943 | Genbank | 7023520 |
| 9137. | AK002038 | Genbank | 7023677 |
| 9138. | AK002100 | Genbank | 7023777 |
| 9139. | AK002149 | Genbank | 7023853 |
| 9140. | AK002157 | Genbank | 7023865 |
| 9141. | AK002173 | Genbank | 7023889 |
| 9142. | AL020990 | Genbank | 3980348 |
| 9143. | AL021546 | Genbank | 2826890 |
| 9144. | AL021940 | Genbank | 3115962 |
| 9145. | AL021997 | Genbank | 3169112 |
| 9146. | AL022101 | Genbank | 3171895 |
| 9147. | AL022166 | Genbank | 4034471 |
| 9148. | AL022170 | Genbank | 3281976 |
| 9149. | AL022312 | Genbank | 4914501 |
| 9150. | AL031295 | Genbank | 4376011 |
| 9151. | AL031347 | Genbank | 4160208 |
| 9152. | AL031432 | Genbank | 4375969 |
| 9153. | AL031591 | Genbank | 6006484 |
| 9154. | AL031670 | Genbank | 4469083 |
| 9155. | AL034379 | Genbank | 5918013 |
| 9156. | AL034582 | Genbank | 5830348 |
| 9157. | AL035086 | Genbank | 4741478 |
| 9158. | AL035209 | Genbank | 4160217 |
| 9159. | AL035409 | Genbank | 5514663 |
| 9160. | AL035422 | Genbank | 5263001 |
| 9161. | AL035593 | Genbank | 5531494 |
| 9162. | AL035658 | Genbank | 4775634 |
| 9163. | AL035661 | Genbank | 6015535 |
| 9164. | AL035666 | Genbank | 4914532 |
| 9165. | AL035701 | Genbank | 5679754 |
| 9166. | AL049176 | Genbank | 4808226 |
| 9167. | AL049397 | Genbank | 4500188 |
| 9168. | AL049404 | Genbank | 4500192 |
| 9169. | AL049795 | Genbank | 6010175 |
| 9170. | AL049830 | Genbank | 6729561 |
| 9171. | AL049844 | Genbank | 5924019 |
| 9172. | AL050022 | Genbank | 4884091 |
| 9173. | AL050037 | Genbank | 4884277 |
| 9174. | AL050087 | Genbank | 4884322 |
| 9175. | AL050091 | Genbank | 4884111 |
| 9176. | AL050197 | Genbank | 4884434 |
| 9177. | AL050205 | Genbank | 4884444 |
| 9178. | AL050228 | Genbank | 4884472 |
| 9179. | AL050254 | Genbank | 4886422 |
| 9180. | AL050262 | Genbank | 4886482 |
| 9181. | AL050331 | Genbank | 5668655 |
| 9182. | AL050341 | Genbank | 5791509 |
| 9183. | AL050373 | Genbank | 4914577 |
| 9184. | AL078599 | Genbank | 5804908 |
| 9185. | AL078612 | Genbank | 5419788 |
| 9186. | AL078621 | Genbank | 6013067 |
| 9187. | AL078638 | Genbank | 6273498 |
| 9188. | AL080102 | Genbank | 5262526 |
| 9189. | AL080113 | Genbank | 5262540 |
| 9190. | AL080206 | Genbank | 5262692 |
| 9191. | AL096710 | Genbank | 5738652 |
| 9192. | AL096719 | Genbank | 5419854 |
| 9193. | AL096740 | Genbank | 5419896 |
| 9194. | AL096801 | Genbank | 6478163 |
| 9195. | AL109614 | Genbank | 8745190 |
| 9196. | AL109758 | Genbank | 8217865 |
| 9197. | AL109847 | Genbank | 6434631 |
| 9198. | AL110140 | Genbank | 5817035 |
| 9199. | AL110141 | Genbank | 5817036 |
| 9200. | AL110183 | Genbank | 5817095 |
| 9201. | AL110197 | Genbank | 5817115 |
| 9202. | AL110297 | Genbank | 5817256 |
| 9203. | AL117237 | Genbank | 5834563 |
| 9204. | AL117412 | Genbank | 5912102 |
| 9205. | AL117513 | Genbank | 5912025 |
| 9206. | AL117590 | Genbank | 5912154 |
| 9207. | AL117595 | Genbank | 5912159 |
| 9208. | AL117602 | Genbank | 5912171 |
| 9209. | AL117664 | Genbank | 5912260 |
| 9210. | AL121919 | Genbank | 8655577 |
| 9211. | AL121997 | Genbank | 7159788 |
| 9212. | AL133215 | Genbank | 7228177 |
| 9213. | AL133216 | Genbank | 6983473 |
| 9214. | AL133245 | Genbank | 7159621 |
| 9215. | AL133396 | Genbank | 6562003 |
| 9216. | AL133544 | Genbank | 7406719 |
| 9217. | AL135744 | Genbank | 6682291 |
| 9218. | AL135858 | Genbank | 7159622 |
| 9219. | AL136543 | Genbank | 6807646 |
| 9220. | AL136593 | Genbank | 7018431 |
| 9221. | AL137163 | Genbank | 6752286 |
| 9222. | AL137269 | Genbank | 6807703 |
| 9223. | AL137321 | Genbank | 6807811 |
| 9224. | AL137327 | Genbank | 6807818 |
| 9225. | AL137330 | Genbank | 6807822 |
| 9226. | AL137515 | Genbank | 6808173 |
| 9227. | AL137543 | Genbank | 6808222 |
| 9228. | AL137663 | Genbank | 6807784 |
| 9229. | AL137861 | Genbank | 9187172 |
| 9230. | AL138752 | Genbank | 8452480 |
| 9231. | AL139825 | Genbank | 8217585 |
| 9232. | AL157361 | Genbank | 7899161 |
| 9233. | AL157427 | Genbank | 7018456 |
| 9234. | AL157438 | Genbank | 7018513 |
| 9235. | AL157482 | Genbank | 7018523 |
| 9236. | AL160191 | Genbank | 7708226 |
| 9237. | AL161968 | Genbank | 7328057 |
| 9238. | AL356596 | Genbank | 8670766 |
| 9239. | AP000024 | Genbank | 3132334 |
| 9240. | AP000065 | Genbank | 4579986 |
| 9241. | AP000217 | Genbank | 4827264 |
| 9242. | AP000496 | Genbank | 5926683 |
| 9243. | AP000535 | Genbank | 5931513 |
| 9244. | AP000554 | Genbank | 5931540 |
| 9245. | AP000555 | Genbank | 5931541 |
| 9246. | AP001597 | Genbank | 7670551 |
| 9247. | D00015 | Genbank | 2618612 |
| 9248. | D00017 | Genbank | 219909 |
| 9249. | D00099 | Genbank | 219941 |
| 9250. | D00137 | Genbank | 219427 |
| 9251. | D10040 | Genbank | 219899 |
| 9252. | D10245 | Genbank | 220069 |
| 9253. | D10924 | Genbank | 219868 |
| 9254. | D11094 | Genbank | 219930 |
| 9255. | D13119 | Genbank | 285909 |
| 9256. | D13315 | Genbank | 219663 |
| 9257. | D13626 | Genbank | 285994 |
| 9258. | D13629 | Genbank | 285984 |
| 9259. | D13630 | Genbank | 286000 |
| 9260. | D13641 | Genbank | 285986 |
| 9261. | D13720 | Genbank | 399657 |
| 9262. | D13892 | Genbank | 474983 |
| 9263. | D13900 | Genbank | 433412 |
| 9264. | D14041 | Genbank | 2326266 |
| 9265. | D14043 | Genbank | 219924 |
| 9266. | D14530 | Genbank | 414348 |
| 9267. | D14661 | Genbank | 285946 |
| 9268. | D14662 | Genbank | 285948 |
| 9269. | D14664 | Genbank | 285952 |
| 9270. | D14696 | Genbank | 285962 |
| 9271. | D14710 | Genbank | 559324 |
| 9272. | D15050 | Genbank | 457560 |
| 9273. | D16224 | Genbank | 532332 |
| 9274. | D16469 | Genbank | 758583 |
| 9275. | D16562 | Genbank | 506336 |
| 9276. | D16919 | Genbank | 598752 |
| 9277. | D23660 | Genbank | 432358 |
| 9278. | D25283 | Genbank | 457443 |
| 9279. | D25542 | Genbank | 662389 |
| 9280. | D26069 | Genbank | 436227 |
| 9281. | D26350 | Genbank | 450468 |
| 9282. | D28463 | Genbank | 461189 |
| 9283. | D29011 | Genbank | 558525 |
| 9284. | D29640 | Genbank | 473930 |
| 9285. | D29954 | Genbank | 473940 |
| 9286. | D31885 | Genbank | 505097 |
| 9287. | D32129 | Genbank | 699597 |
| 9288. | D38112 | Genbank | 644480 |
| 9289. | D38524 | Genbank | 633070 |
| 9290. | D42039 | Genbank | 577290 |
| 9291. | D42047 | Genbank | 577306 |
| 9292. | D43948 | Genbank | 603950 |
| 9293. | D44466 | Genbank | 1808577 |
| 9294. | D45370 | Genbank | 871884 |
| 9295. | D45421 | Genbank | 662289 |
| 9296. | D49355 | Genbank | 1020405 |
| 9297. | D49396 | Genbank | 682747 |
| 9298. | D50372 | Genbank | 2605593 |
| 9299. | D50683 | Genbank | 1827474 |
| 9300. | D50810 | Genbank | 1304118 |
| 9301. | D50916 | Genbank | 1469174 |
| 9302. | D55654 | Genbank | 1255603 |
| 9303. | D55696 | Genbank | 1890049 |
| 9304. | D63486 | Genbank | 1469885 |
| 9305. | D63875 | Genbank | 961441 |
| 9306. | D67031 | Genbank | 2696053 |
| 9307. | D78013 | Genbank | 1330239 |
| 9308. | D78275 | Genbank | 1526425 |
| 9309. | D83018 | Genbank | 1827484 |
| 9310. | D83407 | Genbank | 1435039 |
| 9311. | D83780 | Genbank | 1228042 |
| 9312. | D84476 | Genbank | 1805499 |
| 9313. | D84488 | Genbank | 2388543 |
| 9314. | D84557 | Genbank | 1944481 |
| 9315. | D86724 | Genbank | 1694632 |
| 9316. | D86963 | Genbank | 1503999 |
| 9317. | D87258 | Genbank | 1513058 |
| 9318. | D87434 | Genbank | 1665762 |
| 9319. | D87442 | Genbank | 1665772 |
| 9320. | D87455 | Genbank | 1665798 |
| 9321. | D87666 | Genbank | 1620016 |
| 9322. | D88208 | Genbank | 4586400 |
| 9323. | E01094 | Genbank | 2169353 |
| 9324. | E01246 | Genbank | 2169505 |
| 9325. | E01321 | Genbank | 2169580 |
| 9326. | E01396 | Genbank | 2169652 |
| 9327. | E01497 | Genbank | 2169753 |
| 9328. | E01650 | Genbank | 2169903 |
| 9329. | E01888 | Genbank | 2170137 |
| 9330. | E01956 | Genbank | 2170204 |
| 9331. | E01979 | Genbank | 2170227 |
| 9332. | E02339 | Genbank | 2170574 |
| 9333. | E02628 | Genbank | 2170856 |
| 9334. | E02823 | Genbank | 2171051 |
| 9335. | E05110 | Genbank | 2173304 |
| 9336. | E06721 | Genbank | 2174903 |
| 9337. | E07218 | Genbank | 2175359 |
| 9338. | E08514 | Genbank | 2176629 |
| 9339. | E08663 | Genbank | 2176776 |
| 9340. | E13292 | Genbank | 3252097 |
| 9341. | J00152 | Genbank | 183317 |
| 9342. | J00194 | Genbank | 188231 |
| 9343. | J00196 | Genbank | 188242 |
| 9344. | J00200 | Genbank | 188411 |
| 9345. | J02642 | Genbank | 182862 |
| 9346. | J02763 | Genbank | 179765 |
| 9347. | J02874 | Genbank | 178346 |
| 9348. | J02876 | Genbank | 182413 |
| 9349. | J02902 | Genbank | 189427 |
| 9350. | J02923 | Genbank | 189501 |
| 9351. | J02959 | Genbank | 187174 |
| 9352. | J03005 | Genbank | 183183 |
| 9353. | J03015 | Genbank | 337755 |
| 9354. | J03040 | Genbank | 338312 |
| 9355. | J03132 | Genbank | 184534 |
| 9356. | J03198 | Genbank | 183224 |
| 9357. | J03223 | Genbank | 190419 |
| 9358. | J03507 | Genbank | 179715 |
| 9359. | J03779 | Genbank | 179833 |
| 9360. | J03799 | Genbank | 186840 |
| 9361. | J03801 | Genbank | 187243 |
| 9362. | J03827 | Genbank | 340418 |
| 9363. | J04058 | Genbank | 182250 |
| 9364. | J04080 | Genbank | 179645 |
| 9365. | J04164 | Genbank | 177801 |
| 9366. | J04183 | Genbank | 186929 |
| 9367. | J04478 | Genbank | 179697 |
| 9368. | J04543 | Genbank | 338243 |
| 9369. | J04607 | Genbank | 339666 |
| 9370. | J04621 | Genbank | 184428 |
| 9371. | J04970 | Genbank | 5809681 |
| 9372. | J04977 | Genbank | 186791 |
| 9373. | J04988 | Genbank | 184422 |
| 9374. | J05024 | Genbank | 187824 |
| 9375. | J05036 | Genbank | 181193 |
| 9376. | J05550 | Genbank | 188675 |
| 9377. | K00089 | Genbank | 178422 |
| 9378. | K00558 | Genbank | 340020 |
| 9379. | K00901 | Genbank | 337624 |
| 9380. | K01911 | Genbank | 189273 |
| 9381. | K02885 | Genbank | 338928 |
| 9382. | K03432 | Genbank | 337377 |
| 9383. | L00160 | Genbank | 189904 |
| 9384. | L00212 | Genbank | 190837 |
| 9385. | L00634 | Genbank | 292030 |
| 9386. | L05092 | Genbank | 388031 |
| 9387. | L05093 | Genbank | 401844 |
| 9388. | L05492 | Genbank | 187220 |
| 9389. | L06070 | Genbank | 292509 |
| 9390. | L06237 | Genbank | 437000 |
| 9391. | L07633 | Genbank | 186512 |
| 9392. | L08177 | Genbank | 292056 |
| 9393. | L08895 | Genbank | 292289 |
| 9394. | L09159 | Genbank | 307374 |
| 9395. | L09753 | Genbank | 349277 |
| 9396. | L10091 | Genbank | 185221 |
| 9397. | L10284 | Genbank | 186522 |
| 9398. | L10678 | Genbank | 190387 |
| 9399. | L10717 | Genbank | 307507 |
| 9400. | L10910 | Genbank | 405191 |
| 9401. | L11066 | Genbank | 307322 |
| 9402. | L11670 | Genbank | 180145 |
| 9403. | L12168 | Genbank | 178083 |
| 9404. | L13977 | Genbank | 431320 |
| 9405. | L16785 | Genbank | 349475 |
| 9406. | L19437 | Genbank | 4995970 |
| 9407. | L20941 | Genbank | 507251 |
| 9408. | L22253 | Genbank | 506401 |
| 9409. | L22569 | Genbank | 348706 |
| 9410. | L25085 | Genbank | 459833 |
| 9411. | L27841 | Genbank | 450276 |
| 9412. | L38486 | Genbank | 790816 |
| 9413. | L38941 | Genbank | 1008855 |
| 9414. | L39068 | Genbank | 994714 |
| 9415. | L40386 | Genbank | 703084 |
| 9416. | L41887 | Genbank | 950423 |
| 9417. | L48723 | Genbank | 1246239 |
| 9418. | L49506 | Genbank | 1236234 |
| 9419. | L78440 | Genbank | 1479978 |
| 9420. | M10036 | Genbank | 339840 |
| 9421. | M10119 | Genbank | 182517 |
| 9422. | M11146 | Genbank | 182504 |
| 9423. | M11147 | Genbank | 182513 |
| 9424. | M11315 | Genbank | 180817 |
| 9425. | M11560 | Genbank | 178350 |
| 9426. | M11867 | Genbank | 188229 |
| 9427. | M12759 | Genbank | 532596 |
| 9428. | M12824 | Genbank | 339426 |
| 9429. | M12886 | Genbank | 339009 |
| 9430. | M12937 | Genbank | 182506 |
| 9431. | M12938 | Genbank | 182515 |
| 9432. | M13560 | Genbank | 184517 |
| 9433. | M14058 | Genbank | 179643 |
| 9434. | M14144 | Genbank | 340218 |
| 9435. | M14219 | Genbank | 181169 |
| 9436. | M14630 | Genbank | 339690 |
| 9437. | M15353 | Genbank | 306486 |
| 9438. | M15661 | Genbank | 337577 |
| 9439. | M15856 | Genbank | 187209 |
| 9440. | M15885 | Genbank | 338414 |
| 9441. | M15992 | Genbank | 540443 |
| 9442. | M16086 | Genbank | 540445 |
| 9443. | M16247 | Genbank | 178044 |
| 9444. | M16342 | Genbank | 184266 |
| 9445. | M16597 | Genbank | 181237 |
| 9446. | M16660 | Genbank | 184420 |
| 9447. | M16954 | Genbank | 188243 |
| 9448. | M17733 | Genbank | 339688 |
| 9449. | M17885 | Genbank | 190231 |
| 9450. | M17886 | Genbank | 190233 |
| 9451. | M17987 | Genbank | 179316 |
| 9452. | M19283 | Genbank | 178042 |
| 9453. | M19645 | Genbank | 183644 |
| 9454. | M20430 | Genbank | 188437 |
| 9455. | M21154 | Genbank | 178517 |
| 9456. | M21305 | Genbank | 179068 |
| 9457. | M21574 | Genbank | 189733 |
| 9458. | M21726 | Genbank | 178941 |
| 9459. | M21895 | Genbank | 189523 |
| 9460. | M22382 | Genbank | 190126 |
| 9461. | M22430 | Genbank | 190888 |
| 9462. | M22431 | Genbank | 190885 |
| 9463. | M22918 | Genbank | 189019 |
| 9464. | M22920 | Genbank | 189021 |
| 9465. | M23254 | Genbank | 511636 |
| 9466. | M24194 | Genbank | 187701 |
| 9467. | M24543 | Genbank | 341200 |
| 9468. | M24594 | Genbank | 186262 |
| 9469. | M24795 | Genbank | 178670 |
| 9470. | M24847 | Genbank | 338054 |
| 9471. | M24902 | Genbank | 189618 |
| 9472. | M25246 | Genbank | 340233 |
| 9473. | M25280 | Genbank | 187182 |
| 9474. | M26038 | Genbank | 188303 |
| 9475. | M26663 | Genbank | 618463 |
| 9476. | M26919 | Genbank | 337790 |
| 9477. | M26920 | Genbank | 337791 |
| 9478. | M27394 | Genbank | 179307 |
| 9479. | M27487 | Genbank | 703088 |
| 9480. | M27492 | Genbank | 186289 |
| 9481. | M28209 | Genbank | 550059 |
| 9482. | M28215 | Genbank | 550069 |
| 9483. | M29064 | Genbank | 337452 |
| 9484. | M29696 | Genbank | 186365 |
| 9485. | M29871 | Genbank | 190825 |
| 9486. | M29877 | Genbank | 178408 |
| 9487. | M32019 | Genbank | 181487 |
| 9488. | M32578 | Genbank | 188305 |
| 9489. | M34840 | Genbank | 189620 |
| 9490. | M35368 | Genbank | 1196441 |
| 9491. | M35416 | Genbank | 190851 |
| 9492. | M36501 | Genbank | 177871 |
| 9493. | M37104 | Genbank | 179274 |
| 9494. | M37130 | Genbank | 182926 |
| 9495. | M37197 | Genbank | 179968 |
| 9496. | M37716 | Genbank | 338266 |
| 9497. | M38467 | Genbank | 1160614 |
| 9498. | M55542 | Genbank | 183001 |
| 9499. | M55543 | Genbank | 829176 |
| 9500. | M60457 | Genbank | 181249 |
| 9501. | M60828 | Genbank | 186738 |
| 9502. | M60857 | Genbank | 181334 |
| 9503. | M62840 | Genbank | 178068 |
| 9504. | M63573 | Genbank | 337998 |
| 9505. | M64098 | Genbank | 183891 |
| 9506. | M64241 | Genbank | 190813 |
| 9507. | M64595 | Genbank | 183708 |
| 9508. | M64779 | Genbank | 181449 |
| 9509. | M65181 | Genbank | 177128 |
| 9510. | M69175 | Genbank | 184347 |
| 9511. | M69226 | Genbank | 187354 |
| 9512. | M73548 | Genbank | 190163 |
| 9513. | M74491 | Genbank | 178161 |
| 9514. | M74775 | Genbank | 187151 |
| 9515. | M81757 | Genbank | 337732 |
| 9516. | M81780 | Genbank | 972768 |
| 9517. | M82882 | Genbank | 180551 |
| 9518. | M84349 | Genbank | 180149 |
| 9519. | M86400 | Genbank | 189952 |
| 9520. | M86667 | Genbank | 189066 |
| 9521. | M87789 | Genbank | 185361 |
| 9522. | M87790 | Genbank | 185363 |
| 9523. | M94345 | Genbank | 187455 |
| 9524. | M95601 | Genbank | 178407 |
| 9525. | M95767 | Genbank | 180502 |
| 9526. | M95809 | Genbank | 179568 |
| 9527. | M96322 | Genbank | 183615 |
| 9528. | M96803 | Genbank | 338442 |
| 9529. | M98398 | Genbank | 180110 |
| 9530. | S49994 | Genbank | 260191 |
| 9531. | S50223 | Genbank | 260311 |
| 9532. | S54761 | Genbank | 265221 |
| 9533. | S64477 | Genbank | 408838 |
| 9534. | S67171 | Genbank | 453131 |
| 9535. | S69272 | Genbank | 546087 |
| 9536. | S70290 | Genbank | 546602 |
| 9537. | S72008 | Genbank | 560622 |
| 9538. | S73591 | Genbank | 688296 |
| 9539. | S74017 | Genbank | 693841 |
| 9540. | S74134 | Genbank | 707046 |
| 9541. | S78234 | Genbank | 998471 |
| 9542. | S79522 | Genbank | 243887 |
| 9543. | S79873 | Genbank | 1195541 |
| 9544. | S79895 | Genbank | 1195555 |
| 9545. | U03877 | Genbank | 458227 |
| 9546. | U07086 | Genbank | 515984 |
| 9547. | U07802 | Genbank | 984508 |
| 9548. | U08191 | Genbank | 476273 |
| 9549. | U08212 | Genbank | 487438 |
| 9550. | U09813 | Genbank | 1008454 |
| 9551. | U09848 | Genbank | 495567 |
| 9552. | U10248 | Genbank | 984280 |
| 9553. | U10323 | Genbank | 532312 |
| 9554. | U10485 | Genbank | 505685 |
| 9555. | U10526 | Genbank | 508798 |
| 9556. | U12404 | Genbank | 531170 |
| 9557. | U12789 | Genbank | 555832 |
| 9558. | U14970 | Genbank | 550020 |
| 9559. | U14971 | Genbank | 550022 |
| 9560. | U16306 | Genbank | 608514 |
| 9561. | U16660 | Genbank | 564064 |
| 9562. | U17032 | Genbank | 687592 |
| 9563. | U18121 | Genbank | 915283 |
| 9564. | U18728 | Genbank | 642533 |
| 9565. | U19495 | Genbank | 1754834 |
| 9566. | U23028 | Genbank | 806853 |
| 9567. | U26174 | Genbank | 829637 |
| 9568. | U26424 | Genbank | 1203795 |
| 9569. | U26455 | Genbank | 870785 |
| 9570. | U27109 | Genbank | 927595 |
| 9571. | U27460 | Genbank | 881393 |
| 9572. | U28042 | Genbank | 1142709 |
| 9573. | U29091 | Genbank | 1374791 |
| 9574. | U29615 | Genbank | 1050957 |
| 9575. | U30246 | Genbank | 903681 |
| 9576. | U30521 | Genbank | 963091 |
| 9577. | U30888 | Genbank | 940181 |
| 9578. | U31656 | Genbank | 951330 |
| 9579. | U31906 | Genbank | 1173564 |
| 9580. | U32519 | Genbank | 1051169 |
| 9581. | U33760 | Genbank | 995823 |
| 9582. | U36764 | Genbank | 1036804 |
| 9583. | U37143 | Genbank | 1185451 |
| 9584. | U41060 | Genbank | 1256000 |
| 9585. | U41515 | Genbank | 1209723 |
| 9586. | U43701 | Genbank | 1399085 |
| 9587. | U46006 | Genbank | 1314358 |
| 9588. | U46837 | Genbank | 1197662 |
| 9589. | U47105 | Genbank | 4457236 |
| 9590. | U47924 | Genbank | 1633547 |
| 9591. | U53225 | Genbank | 1293679 |
| 9592. | U54559 | Genbank | 2351379 |
| 9593. | U54562 | Genbank | 2351381 |
| 9594. | U54831 | Genbank | 1354506 |
| 9595. | U55937 | Genbank | 1575044 |
| 9596. | U57646 | Genbank | 1373337 |
| 9597. | U57847 | Genbank | 1373420 |
| 9598. | U59185 | Genbank | 2463627 |
| 9599. | U59209 | Genbank | 3287472 |
| 9600. | U59305 | Genbank | 1695872 |
| 9601. | U59435 | Genbank | 2697004 |
| 9602. | U59863 | Genbank | 1518017 |
| 9603. | U62740 | Genbank | 1518041 |
| 9604. | U63830 | Genbank | 1621610 |
| 9605. | U65676 | Genbank | 1654350 |
| 9606. | U66616 | Genbank | 1549240 |
| 9607. | U67156 | Genbank | 1679667 |
| 9608. | U67171 | Genbank | 2326174 |
| 9609. | U69274 | Genbank | 4097821 |
| 9610. | U70063 | Genbank | 1743866 |
| 9611. | U70323 | Genbank | 1679683 |
| 9612. | U72935 | Genbank | 1778349 |
| 9613. | U73529 | Genbank | 2209284 |
| 9614. | U73824 | Genbank | 1857236 |
| 9615. | U79258 | Genbank | 1710211 |
| 9616. | U79415 | Genbank | 1947070 |
| 9617. | U83194 | Genbank | 4580012 |
| 9618. | U88320 | Genbank | 2196919 |
| 9619. | U90549 | Genbank | 2062699 |
| 9620. | U91323 | Genbank | 3582311 |
| 9621. | U96113 | Genbank | 2072500 |
| 9622. | V00518 | Genbank | 31868 |
| 9623. | V00710 | Genbank | 13683 |
| 9624. | X01742 | Genbank | 35324 |
| 9625. | X01750 | Genbank | 28868 |
| 9626. | X02152 | Genbank | 34312 |
| 9627. | X03205 | Genbank | 36162 |
| 9628. | X03350 | Genbank | 28415 |
| 9629. | X03693 | Genbank | 35978 |
| 9630. | X03742 | Genbank | 28518 |
| 9631. | X04011 | Genbank | 37983 |
| 9632. | X04098 | Genbank | 28338 |
| 9633. | X04588 | Genbank | 37423 |
| 9634. | X07466 | Genbank | 31769 |
| 9635. | X13238 | Genbank | 1200056 |
| 9636. | X14303 | Genbank | 36360 |
| 9637. | X14420 | Genbank | 30057 |
| 9638. | X15729 | Genbank | 38317 |
| 9639. | X15822 | Genbank | 30146 |
| 9640. | X17115 | Genbank | 33450 |
| 9641. | X51405 | Genbank | 29666 |
| 9642. | X51408 | Genbank | 35012 |
| 9643. | X52142 | Genbank | 30292 |
| 9644. | X52856 | Genbank | 30160 |
| 9645. | X52947 | Genbank | 29916 |
| 9646. | X55733 | Genbank | 288099 |
| 9647. | X56158 | Genbank | 37724 |
| 9648. | X56932 | Genbank | 23690 |
| 9849. | X57025 | Genbank | 33007 |
| 9650. | X57129 | Genbank | 31967 |
| 9651. | X57351 | Genbank | 311373 |
| 9652. | X57352 | Genbank | 311374 |
| 9653. | X59417 | Genbank | 35681 |
| 9654. | X59710 | Genbank | 35049 |
| 9655. | X59969 | Genbank | 29451 |
| 9656. | X60221 | Genbank | 509290 |
| 9657. | X60489 | Genbank | 31099 |
| 9658. | X60656 | Genbank | 31134 |
| 9659. | X61615 | Genbank | 34365 |
| 9660. | X63237 | Genbank | 37570 |
| 9661. | X63432 | Genbank | 28335 |
| 9662. | X65873 | Genbank | 34082 |
| 9663. | X65923 | Genbank | 31302 |
| 9664. | X66401 | Genbank | 34634 |
| 9665. | X66533 | Genbank | 31685 |
| 9666. | X67698 | Genbank | 37476 |
| 9667. | X67906 | Genbank | 33582 |
| 9668. | X69086 | Genbank | 34811 |
| 9669. | X70421 | Genbank | 33068 |
| 9670. | X71125 | Genbank | 398375 |
| 9671. | X71635 | Genbank | 297099 |
| 9672. | X71810 | Genbank | 297905 |
| 9673. | X74801 | Genbank | 671526 |
| 9674. | X74968 | Genbank | 439659 |
| 9675. | X76534 | Genbank | 666042 |
| 9676. | X77548 | Genbank | 469145 |
| 9677. | X77744 | Genbank | 456189 |
| 9678. | X78627 | Genbank | 607129 |
| 9679. | X80062 | Genbank | 663208 |
| 9680. | X80303 | Genbank | 516141 |
| 9681. | X81695 | Genbank | 940515 |
| 9682. | X81696 | Genbank | 940516 |
| 9683. | X82200 | Genbank | 899299 |
| 9684. | X82321 | Genbank | 1617117 |
| 9685. | X82390 | Genbank | 565028 |
| 9686. | X83218 | Genbank | 1008079 |
| 9687. | X84908 | Genbank | 1502344 |
| 9688. | X85018 | Genbank | 971458 |
| 9689. | X85117 | Genbank | 1161563 |
| 9690. | X85372 | Genbank | 806563 |
| 9691. | X92689 | Genbank | 1296629 |
| 9692. | X93920 | Genbank | 1418933 |
| 9693. | X94323 | Genbank | 1213612 |
| 9694. | X94910 | Genbank | 3413292 |
| 9695. | X99585 | Genbank | 1770518 |
| 9696. | Y00062 | Genbank | 34275 |
| 9697. | Y00281 | Genbank | 36052 |
| 9698. | Y00345 | Genbank | 35569 |
| 9699. | Y00371 | Genbank | 32466 |
| 9700. | Y00815 | Genbank | 34266 |
| 9701. | Y08915 | Genbank | 1877201 |
| 9702. | Y09022 | Genbank | 1653999 |
| 9703. | Y09328 | Genbank | 1885307 |
| 9704. | Y11215 | Genbank | 2252495 |
| 9705. | Y12065 | Genbank | 2230877 |
| 9706. | Y13286 | Genbank | 2853173 |
| 9707. | Y13620 | Genbank | 2181877 |
| 9708. | Y14737 | Genbank | 2765424 |
| 9709. | Y16414 | Genbank | 2924334 |
| 9710. | Y17957 | Genbank | 10241692 |
| 9711. | Y18314 | Genbank | 5457356 |
| 9712. | Z11793 | Genbank | 36425 |
| 9713. | Z12006 | Genbank | 28884 |
| 9714. | Z12011 | Genbank | 28888 |
| 9715. | Z12012 | Genbank | 28889 |
| 9716. | Z14070 | Genbank | 35822 |
| 9717. | Z14195 | Genbank | 30977 |
| 9718. | Z47087 | Genbank | 860989 |
| 9719. | Z47727 | Genbank | 717186 |
| 9720. | Z48042 | Genbank | 662993 |
| 9721. | Z48501 | Genbank | 693936 |
| 9722. | Z48950 | Genbank | 761715 |
| 9723. | Z50101 | Genbank | 1000703 |
| 9724. | Z61867 | Genbank | 1034245 |
| 9725. | Z62968 | Genbank | 1035346 |
| 9726. | Z73639 | Genbank | 2632099 |
| 9727. | Z73965 | Genbank | 1370122 |
| 9728. | Z77853 | Genbank | 1483352 |
| 9729. | Z79305 | Genbank | 1508583 |
| 9730. | Z81326 | Genbank | 1785653 |
| 9731. | Z82188 | Genbank | 5102611 |
| 9732. | Z85986 | Genbank | 4034056 |
| 9733. | Z95114 | Genbank | 5101742 |
| 9734. | Z95125 | Genbank | 2440066 |
| 9735. | Z98036 | Genbank | 2578066 |
| 9736. | Z98049 | Genbank | 2462401 |
| 9737. | Z98200 | Genbank | 6002294 |
| 9738. | Z98716 | Genbank | 2340903 |
| 9739. | Z98884 | Genbank | 5304861 |
| 9740. | AA001673 | EST | 1445230 |
| 9741. | AA004905 | EST | 1448365 |
| 9742. | AA005233 | EST | 1448695 |
| 9743. | AA017175 | EST | 1479340 |
| 9744. | AA017533 | EST | 1479686 |
| 9745. | AA029143 | EST | 1496545 |
| 9746. | AA029569 | EST | 1497037 |
| 9747. | AA029679 | EST | 1497082 |
| 9748. | AA031703 | EST | 1501685 |
| 9749. | AA031824 | EST | 1501787 |
| 9750. | AA035003 | EST | 1506966 |
| 9751. | AA035421 | EST | 1507078 |
| 9752. | AA035777 | EST | 1507605 |
| 9753. | AA036712 | EST | 1509760 |
| 9754. | AA037179 | EST | 1512323 |
| 9755. | AA039948 | EST | 1516243 |
| 9756. | AA039967 | EST | 1516280 |
| 9757. | AA040130 | EST | 1516408 |
| 9758. | AA040144 | EST | 1516422 |
| 9759. | AA040405 | EST | 1516766 |
| 9760. | AA041467 | EST | 1517765 |
| 9761. | AA043141 | EST | 1520995 |
| 9762. | AA043259 | EST | 1521133 |
| 9763. | AA043503 | EST | 1521427 |
| 9764. | AA044586 | EST | 1522977 |
| 9765. | AA045949 | EST | 1525843 |
| 9766. | AA046077 | EST | 1526026 |
| 9767. | AA055649 | EST | 1547988 |
| 9768. | AA056104 | EST | 1548441 |
| 9769. | AA056121 | EST | 1548670 |
| 9770. | AA056182 | EST | 1548520 |
| 9771. | AA056358 | EST | 1548698 |
| 9772. | AA058906 | EST | 1551740 |
| 9773. | AA062552 | EST | 1556726 |
| 9774. | AA062844 | EST | 1557469 |
| 9775. | AA063373 | EST | 1557041 |
| 9776. | AA069784 | EST | 1577152 |
| 9777. | AA074792 | EST | 1614679 |
| 9778. | AA075752 | EST | 1615817 |
| 9779. | AA075781 | EST | 1615653 |
| 9780. | AA075787 | EST | 1615659 |
| 9781. | AA076290 | EST | 1616159 |
| 9782. | AA082157 | EST | 1624278 |
| 9783. | AA082800 | EST | 1624857 |
| 9784. | AA082856 | EST | 1624975 |
| 9785. | AA083585 | EST | 1625645 |
| 9786. | AA083950 | EST | 1626024 |
| 9787. | AA084009 | EST | 1626066 |
| 9788. | AA084560 | EST | 1626616 |
| 9789. | AA084885 | EST | 1626942 |
| 9790. | AA085281 | EST | 1627408 |
| 9791. | AA088671 | EST | 1634192 |
| 9792. | AA091539 | EST | 1637002 |
| 9793. | AA092847 | EST | 1637860 |
| 9794. | AA094871 | EST | 1640456 |
| 9795. | AA098789 | EST | 1644759 |
| 9796. | AA099195 | EST | 1645712 |
| 9797. | AA099520 | EST | 1645466 |
| 9798. | AA099567 | EST | 1645584 |
| 9799. | AA099976 | EST | 1646109 |
| 9800. | AA102564 | EST | 1647756 |
| 9801. | AA102609 | EST | 1647895 |
| 9802. | AA112893 | EST | 1664244 |
| 9803. | AA114070 | EST | 1668135 |
| 9804. | AA114890 | EST | 1670003 |
| 9805. | AA114984 | EST | 1670079 |
| 9806. | AA126666 | EST | 1686222 |
| 9807. | AA127737 | EST | 1687100 |
| 9808. | AA130200 | EST | 1691408 |
| 9809. | AA132199 | EST | 1693690 |
| 9810. | AA143151 | EST | 1712521 |
| 9811. | AA143300 | EST | 1712671 |
| 9812. | AA146994 | EST | 1716363 |
| 9813. | AA147833 | EST | 1717205 |
| 9814. | AA148771 | EST | 1721626 |
| 9815. | AA148773 | EST | 1721628 |
| 9816. | AA148822 | EST | 1721659 |
| 9817. | AA150422 | EST | 1721935 |
| 9818. | AA151483 | EST | 1719988 |
| 9819. | AA151687 | EST | 1720242 |
| 9820. | AA152264 | EST | 1721601 |
| 9821. | AA152396 | EST | 1718607 |
| 9822. | AA155865 | EST | 1727500 |
| 9823. | AA156847 | EST | 1728499 |
| 9824. | AA160053 | EST | 1734518 |
| 9825. | AA160066 | EST | 1734531 |
| 9826. | AA160278 | EST | 1734855 |
| 9827. | AA161490 | EST | 1735800 |
| 9828. | AA161515 | EST | 1735825 |
| 9829. | AA165662 | EST | 1741713 |
| 9830. | AA166628 | EST | 1745092 |
| 9831. | AA166908 | EST | 1745418 |
| 9832. | AA169404 | EST | 1748344 |
| 9833. | AA172039 | EST | 1751096 |
| 9834. | AA173045 | EST | 1754315 |
| 9835. | AA173098 | EST | 1753340 |
| 9836. | AA173470 | EST | 1753798 |
| 9837. | AA173559 | EST | 1753691 |
| 9838. | AA178874 | EST | 1760397 |
| 9839. | AA179711 | EST | 1761134 |
| 9840. | AA180517 | EST | 1761790 |
| 9841. | AA180914 | EST | 1764390 |
| 9842. | AA181288 | EST | 1764754 |
| 9843. | AA181618 | EST | 1765085 |
| 9844. | AA187106 | EST | 1775216 |
| 9845. | AA188045 | EST | 1774295 |
| 9846. | AA188875 | EST | 1775902 |
| 9847. | AA190575 | EST | 1779550 |
| 9848. | AA192304 | EST | 1781525 |
| 9849. | AA192374 | EST | 1781595 |
| 9850. | AA194946 | EST | 1784637 |
| 9851. | AA195119 | EST | 1784830 |
| 9852. | AA195815 | EST | 1791397 |
| 9853. | AA196223 | EST | 1791805 |
| 9854. | AA196448 | EST | 1792079 |
| 9855. | AA199662 | EST | 1795369 |
| 9856. | AA199896 | EST | 1795630 |
| 9857. | AA203152 | EST | 1798878 |
| 9858. | AA203251 | EST | 1798969 |
| 9859. | AA203274 | EST | 1798992 |
| 9860. | AA215680 | EST | 1815443 |
| 9861. | AA215830 | EST | 1815584 |
| 9862. | AA221006 | EST | 1839749 |
| 9863. | AA224042 | EST | 1844583 |
| 9864. | AA224123 | EST | 1844682 |
| 9865. | AA224876 | EST | 1846242 |
| 9866. | AA226850 | EST | 1848385 |
| 9867. | AA227413 | EST | 1848975 |
| 9868. | AA232128 | EST | 1855613 |
| 9869. | AA233292 | EST | 1856285 |
| 9870. | AA244003 | EST | 1874726 |
| 9871. | AA251109 | EST | 1886071 |
| 9872. | AA251165 | EST | 1886173 |
| 9873. | AA251598 | EST | 1886623 |
| 9874. | AA251850 | EST | 1886812 |
| 9875. | AA258389 | EST | 1893521 |
| 9876. | AA258963 | EST | 1894088 |
| 9877. | AA262132 | EST | 1898258 |
| 9878. | AA262508 | EST | 1898003 |
| 9879. | AA278524 | EST | 1919862 |
| 9880. | AA280235 | EST | 1921773 |
| 9881. | AA280328 | EST | 1921866 |
| 9882. | AA280671 | EST | 1923532 |
| 9883. | AA281220 | EST | 1924107 |
| 9884. | AA281527 | EST | 1924207 |
| 9885. | AA282903 | EST | 1925836 |
| 9886. | AA286702 | EST | 1933584 |
| 9887. | AA286805 | EST | 1933668 |
| 9888. | AA292011 | EST | 1939988 |
| 9889. | AA292443 | EST | 1940422 |
| 9890. | AA293028 | EST | 1940924 |
| 9891. | AA293759 | EST | 1941542 |
| 9892. | AA295420 | EST | 1947828 |
| 9893. | AA298475 | EST | 1950818 |
| 9894. | AA298941 | EST | 1951324 |
| 9895. | AA300705 | EST | 1953038 |
| 9896. | AA302521 | EST | 1955094 |
| 9897. | AA303072 | EST | 1955402 |
| 9898. | AA303184 | EST | 1955537 |
| 9899. | AA303300 | EST | 1955634 |
| 9900. | AA304947 | EST | 1957274 |
| 9901. | AA304964 | EST | 1957291 |
| 9902. | AA305253 | EST | 1957641 |
| 9903. | AA307313 | EST | 1959713 |
| 9904. | AA307555 | EST | 1960104 |
| 9905. | AA307720 | EST | 1960048 |
| 9906. | AA307779 | EST | 1960177 |
| 9907. | AA308281 | EST | 1960610 |
| 9908. | AA310570 | EST | 1962917 |
| 9909. | AA310946 | EST | 1963274 |
| 9910. | AA311227 | EST | 1963627 |
| 9911. | AA312054 | EST | 1964424 |
| 9912. | AA312870 | EST | 1965218 |
| 9913. | AA313692 | EST | 1966021 |
| 9914. | AA314472 | EST | 1966821 |
| 9915. | AA315010 | EST | 1967340 |
| 9916. | AA315348 | EST | 1967697 |
| 9917. | AA315384 | EST | 1967712 |
| 9918. | AA315511 | EST | 1967860 |
| 9919. | AA316089 | EST | 1968579 |
| 9920. | AA316423 | EST | 1968973 |
| 9921. | AA316546 | EST | 1968874 |
| 9922. | AA316681 | EST | 1969009 |
| 9923. | AA316893 | EST | 1969465 |
| 9924. | AA316962 | EST | 1969310 |
| 9925. | AA318372 | EST | 1970858 |
| 9926. | AA318934 | EST | 1971260 |
| 9927. | AA323618 | EST | 1975944 |
| 9928. | AA324270 | EST | 1976513 |
| 9929. | AA324751 | EST | 1977037 |
| 9930. | AA328843 | EST | 1981087 |
| 9931. | AA332856 | EST | 1985162 |
| 9932. | AA333526 | EST | 1985779 |
| 9933. | AA334020 | EST | 1986264 |
| 9934. | AA335771 | EST | 1988012 |
| 9935. | AA336842 | EST | 1989081 |
| 9936. | AA339643 | EST | 1992035 |
| 9937. | AA340927 | EST | 1993166 |
| 9938. | AA343749 | EST | 1995986 |
| 9939. | AA344621 | EST | 1996857 |
| 9940. | AA345836 | EST | 1998073 |
| 9941. | AA347572 | EST | 1999810 |
| 9942. | AA348987 | EST | 2001223 |
| 9943. | AA349541 | EST | 2001780 |
| 9944. | AA349688 | EST | 2002028 |
| 9945. | AA354891 | EST | 2007210 |
| 9946. | AA357445 | EST | 2009774 |
| 9947. | AA360212 | EST | 2012532 |
| 9948. | AA364309 | EST | 2016627 |
| 9949. | AA366087 | EST | 2018468 |
| 9950. | AA366995 | EST | 2019354 |
| 9951. | AA370116 | EST | 2022432 |
| 9952. | AA370279 | EST | 2022816 |
| 9953. | AA377218 | EST | 2029535 |
| 9954. | AA397468 | EST | 2050509 |
| 9955. | AA398819 | EST | 2050918 |
| 9956. | AA404281 | EST | 2059005 |
| 9957. | AA404673 | EST | 2058911 |
| 9958. | AA410818 | EST | 2069976 |
| 9959. | AA416946 | EST | 2076992 |
| 9960. | AA417772 | EST | 2079652 |
| 9961. | AA418052 | EST | 2079889 |
| 9962. | AA418770 | EST | 2080571 |
| 9963. | AA419457 | EST | 2079209 |
| 9964. | AA420624 | EST | 2094502 |
| 9965. | AA420758 | EST | 2094637 |
| 9966. | AA421065 | EST | 2099880 |
| 9967. | AA421118 | EST | 2099943 |
| 9968. | AA421303 | EST | 2100145 |
| 9969. | AA421911 | EST | 2100728 |
| 9970. | AA422079 | EST | 2100903 |
| 9971. | AA422171 | EST | 2101022 |
| 9972. | AA424365 | EST | 2103326 |
| 9973. | AA424980 | EST | 2107133 |
| 9974. | AA425400 | EST | 2106156 |
| 9975. | AA428544 | EST | 2112756 |
| 9976. | AA428694 | EST | 2110272 |
| 9977. | AA429538 | EST | 2112638 |
| 9978. | AA429938 | EST | 2113147 |
| 9979. | AA431048 | EST | 2113768 |
| 9980. | AA435620 | EST | 2140534 |
| 9981. | AA436116 | EST | 2141030 |
| 9982. | AA441787 | EST | 2153671 |
| 9983. | AA442040 | EST | 2153918 |
| 9984. | AA442925 | EST | 2155600 |
| 9985. | AA443496 | EST | 2156171 |
| 9986. | AA445937 | EST | 2158602 |
| 9987. | AA446083 | EST | 2158748 |
| 9988. | AA446158 | EST | 2158823 |
| 9989. | AA446282 | EST | 2158947 |
| 9990. | AA448618 | EST | 2162288 |
| 9991. | AA448965 | EST | 2162985 |
| 9992. | AA449236 | EST | 2162699 |
| 9993. | AA450163 | EST | 2163913 |
| 9994. | AA452500 | EST | 2166169 |
| 9995. | AA456527 | EST | 2179103 |
| 9996. | AA461228 | EST | 2186348 |
| 9997. | AA463819 | EST | 2188703 |
| 9998. | AA476405 | EST | 2204616 |
| 9999. | AA478669 | EST | 2207303 |
| 10000. | AA478889 | EST | 2207523 |
| 10001. | AA479306 | EST | 2207862 |
| 10002. | AA480178 | EST | 2208329 |
| 10003. | AA481177 | EST | 2210729 |
| 10004. | AA481485 | EST | 2211037 |
| 10005. | AA481979 | EST | 2209657 |
| 10006. | AA482212 | EST | 2209890 |
| 10007. | AA483243 | EST | 2212056 |
| 10008. | AA484752 | EST | 2214137 |
| 10009. | AA487483 | EST | 2217647 |
| 10010. | AA488006 | EST | 2215437 |
| 10011. | AA489527 | EST | 2219129 |
| 10012. | AA490220 | EST | 2219402 |
| 10013. | AA491601 | EST | 2221163 |
| 10014. | AA492280 | EST | 2221842 |
| 10015. | AA492492 | EST | 2222054 |
| 10016. | AA495988 | EST | 2229309 |
| 10017. | AA496082 | EST | 2229403 |
| 10018. | AA501770 | EST | 2236737 |
| 10019. | AA502979 | EST | 2237946 |
| 10020. | AA503861 | EST | 2238828 |
| 10021. | AA504404 | EST | 2240564 |
| 10022. | AA504813 | EST | 2240973 |
| 10023. | AA505884 | EST | 2242021 |
| 10024. | AA507867 | EST | 2244306 |
| 10025. | AA515994 | EST | 2255594 |
| 10026. | AA521127 | EST | 2261670 |
| 10027. | AA523252 | EST | 2263964 |
| 10028. | AA523498 | EST | 2264210 |
| 10029. | AA526057 | EST | 2268126 |
| 10030. | AA527298 | EST | 2269367 |
| 10031. | AA527737 | EST | 2269806 |
| 10032. | AA527805 | EST | 2269874 |
| 10033. | AA528106 | EST | 2270175 |
| 10034. | AA528645 | EST | 2270714 |
| 10035. | AA533057 | EST | 2277153 |
| 10036. | AA533271 | EST | 2277367 |
| 10037. | AA534891 | EST | 2279144 |
| 10038. | AA535762 | EST | 2280015 |
| 10039. | AA535790 | EST | 2280043 |
| 10040. | AA548284 | EST | 2318566 |
| 10041. | AA548658 | EST | 2318940 |
| 10042. | AA551536 | EST | 2321788 |
| 10043. | AA552858 | EST | 2323112 |
| 10044. | AA553826 | EST | 2324365 |
| 10045. | AA557127 | EST | 2327604 |
| 10046. | AA569895 | EST | 2343875 |
| 10047. | AA572689 | EST | 2347218 |
| 10048. | AA572934 | EST | 2347462 |
| 10049. | AA573364 | EST | 2347892 |
| 10050. | AA573645 | EST | 2360881 |
| 10051. | AA573762 | EST | 2348277 |
| 10052. | AA573952 | EST | 2348467 |
| 10053. | AA574022 | EST | 2348537 |
| 10054. | AA575933 | EST | 2350448 |
| 10055. | AA576547 | EST | 2354047 |
| 10056. | AA578577 | EST | 2356761 |
| 10057. | AA580417 | EST | 2355744 |
| 10058. | AA582853 | EST | 2360213 |
| 10059. | AA585392 | EST | 2385280 |
| 10060. | AA587874 | EST | 2402049 |
| 10061. | AA587917 | EST | 2402092 |
| 10062. | AA588844 | EST | 2401274 |
| 10063. | AA592908 | EST | 2408670 |
| 10064. | AA595117 | EST | 2410467 |
| 10065. | AA595471 | EST | 2410821 |
| 10066. | AA598697 | EST | 2432369 |
| 10067. | AA599881 | EST | 2433506 |
| 10068. | AA600212 | EST | 2433837 |
| 10069. | AA601882 | EST | 2436035 |
| 10070. | AA602357 | EST | 2436335 |
| 10071. | AA602957 | EST | 2436009 |
| 10072. | AA603107 | EST | 2436968 |
| 10073. | AA603219 | EST | 2437080 |
| 10074. | AA604229 | EST | 2445138 |
| 10075. | AA609521 | EST | 2457949 |
| 10076. | AA620750 | EST | 2524689 |
| 10077. | AA620932 | EST | 2524871 |
| 10078. | AA621970 | EST | 2525846 |
| 10079. | AA622735 | EST | 2526611 |
| 10080. | AA627344 | EST | 2540361 |
| 10081. | AA627550 | EST | 2539645 |
| 10082. | AA630741 | EST | 2553352 |
| 10083. | AA632226 | EST | 2555640 |
| 10084. | AA632742 | EST | 2556156 |
| 10085. | AA633630 | EST | 2556844 |
| 10086. | AA633918 | EST | 2557132 |
| 10087. | AA641659 | EST | 2566877 |
| 10088. | AA642033 | EST | 2567251 |
| 10089. | AA649075 | EST | 2575504 |
| 10090. | AA654557 | EST | 2590711 |
| 10091. | AA657516 | EST | 2593670 |
| 10092. | AA657677 | EST | 2593831 |
| 10093. | AA659719 | EST | 2595873 |
| 10094. | AA662920 | EST | 2616911 |
| 10095. | AA668865 | EST | 2630364 |
| 10096. | AA682310 | EST | 2669627 |
| 10097. | AA703606 | EST | 2713524 |
| 10098. | AA707351 | EST | 2717269 |
| 10099. | AA730124 | EST | 2751406 |
| 10100. | AA743621 | EST | 2783127 |
| 10101. | AA745592 | EST | 2785578 |
| 10102. | AA746366 | EST | 2786352 |
| 10103. | AA748038 | EST | 2787996 |
| 10104. | AA760966 | EST | 2809896 |
| 10105. | AA766596 | EST | 2817834 |
| 10106. | AA767033 | EST | 2819614 |
| 10107. | AA769622 | EST | 2820860 |
| 10108. | AA772439 | EST | 2824222 |
| 10109. | AA773062 | EST | 2824633 |
| 10110. | AA773462 | EST | 2825033 |
| 10111. | AA774273 | EST | 2825571 |
| 10112. | AA776459 | EST | 2835793 |
| 10113. | AA777014 | EST | 2836345 |
| 10114. | AA779515 | EST | 2838846 |
| 10115. | AA780836 | EST | 2840167 |
| 10116. | AA781073 | EST | 2840404 |
| 10117. | AA806561 | EST | 2875311 |
| 10118. | AA809057 | EST | 2878463 |
| 10119. | AA809889 | EST | 2879295 |
| 10120. | AA813108 | EST | 2883093 |
| 10121. | AA814672 | EST | 2884268 |
| 10122. | AA827291 | EST | 2899732 |
| 10123. | AA827878 | EST | 2900241 |
| 10124. | AA829527 | EST | 2902626 |
| 10125. | AA829760 | EST | 2902859 |
| 10126. | AA834873 | EST | 2908472 |
| 10127. | AA835500 | EST | 2909228 |
| 10128. | AA836965 | EST | 2912164 |
| 10129. | AA877458 | EST | 2985165 |
| 10130. | AA883673 | EST | 2993203 |
| 10131. | AA887453 | EST | 3003141 |
| 10132. | AA888059 | EST | 3003734 |
| 10133. | AA888097 | EST | 3003772 |
| 10134. | AA889422 | EST | 3016301 |
| 10135. | AA902995 | EST | 3038118 |
| 10136. | AA906427 | EST | 3041550 |
| 10137. | AA906920 | EST | 3042380 |
| 10138. | AA918127 | EST | 3058017 |
| 10139. | AA928143 | EST | 3077299 |
| 10140. | AA931528 | EST | 3085914 |
| 10141. | AA935806 | EST | 3092963 |
| 10142. | AA937773 | EST | 3095884 |
| 10143. | AA939102 | EST | 3099015 |
| 10144. | AA984134 | EST | 3162659 |
| 10145. | AA992606 | EST | 3179362 |
| 10146. | AA992643 | EST | 3178377 |
| 10147. | AA995028 | EST | 3181517 |
| 10148. | AF001541 | EST | 2529713 |
| 10149. | AF001542 | EST | 2529714 |
| 10150. | AI002304 | EST | 3202638 |
| 10151. | AI003783 | EST | 3213293 |
| 10152. | AI017689 | EST | 3232025 |
| 10153. | AI017925 | EST | 3232261 |
| 10154. | AI018166 | EST | 3232685 |
| 10155. | AI018522 | EST | 3233041 |
| 10156. | AI021964 | EST | 3237577 |
| 10157. | AI024860 | EST | 3240473 |
| 10158. | AI025735 | EST | 3241348 |
| 10159. | AI031979 | EST | 3250191 |
| 10160. | AI041328 | EST | 3280522 |
| 10161. | AI042017 | EST | 3281211 |
| 10162. | AI057009 | EST | 3330798 |
| 10163. | AI064691 | EST | 6358963 |
| 10164. | AI066497 | EST | 3367199 |
| 10165. | AI073501 | EST | 3400145 |
| 10166. | AI079233 | EST | 3415484 |
| 10167. | AI080476 | EST | 3416727 |
| 10168. | AI081356 | EST | 3418148 |
| 10169. | AI085616 | EST | 3424039 |
| 10170. | AI086530 | EST | 3424953 |
| 10171. | AI088454 | EST | 3427513 |
| 10172. | AI090600 | EST | 3429659 |
| 10173. | AI092761 | EST | 3431737 |
| 10174. | AI095375 | EST | 3434351 |
| 10175. | AI096521 | EST | 3446015 |
| 10176. | AI110753 | EST | 6359618 |
| 10177. | AI125169 | EST | 3593683 |
| 10178. | AI128839 | EST | 3597353 |
| 10179. | AI129714 | EST | 3598228 |
| 10180. | AI130843 | EST | 3600859 |
| 10181. | AI133208 | EST | 6360524 |
| 10182. | AI133613 | EST | 6360929 |
| 10183. | AI139036 | EST | 3645008 |
| 10184. | AI139652 | EST | 3645624 |
| 10185. | AI139990 | EST | 3647447 |
| 10186. | AI143319 | EST | 3665128 |
| 10187. | AI148306 | EST | 3675988 |
| 10188. | AI149825 | EST | 3678294 |
| 10189. | AI160114 | EST | 3693494 |
| 10190. | AI174675 | EST | 6361053 |
| 10191. | AI174956 | EST | 6361364 |
| 10192. | AI189911 | EST | 3741120 |
| 10193. | AI191990 | EST | 3743199 |
| 10194. | AI192200 | EST | 3743409 |
| 10195. | AI198311 | EST | 3750917 |
| 10196. | AI202490 | EST | 3755096 |
| 10197. | AI204698 | EST | 3757304 |
| 10198. | AI206663 | EST | 3765335 |
| 10199. | AI215617 | EST | 3784658 |
| 10200. | AI217003 | EST | 3789657 |
| 10201. | AI246688 | EST | 3842085 |
| 10202. | AI250797 | EST | 3847326 |
| 10203. | AI251963 | EST | 3848492 |
| 10204. | AI253330 | EST | 3850451 |
| 10205. | AI253395 | EST | 3850350 |
| 10206. | AI253436 | EST | 3850391 |
| 10207. | AI267158 | EST | 3886325 |
| 10208. | AI267185 | EST | 3886352 |
| 10209. | AI267270 | EST | 3886437 |
| 10210. | AI267282 | EST | 3886449 |
| 10211. | AI267289 | EST | 3886456 |
| 10212. | AI267391 | EST | 3886558 |
| 10213. | AI267395 | EST | 3886562 |
| 10214. | AI267397 | EST | 3886564 |
| 10215. | AI267405 | EST | 3886572 |
| 10216. | AI267440 | EST | 3886607 |
| 10217. | AI267664 | EST | 3886831 |
| 10218. | AI269642 | EST | 3888809 |
| 10219. | AI270418 | EST | 3889585 |
| 10220. | AI273945 | EST | 3896213 |
| 10221. | AI274084 | EST | 3896352 |
| 10222. | AI275850 | EST | 3898124 |
| 10223. | AI276450 | EST | 3898724 |
| 10224. | AI278024 | EST | 3900292 |
| 10225. | AI278129 | EST | 3900397 |
| 10226. | AI279897 | EST | 3918131 |
| 10227. | AI281509 | EST | 3919742 |
| 10228. | AI290833 | EST | 3933607 |
| 10229. | AI300188 | EST | 3959534 |
| 10230. | AI309431 | EST | 4004302 |
| 10231. | AI309564 | EST | 4004435 |
| 10232. | AI338757 | EST | 4075684 |
| 10233. | AI339463 | EST | 4076390 |
| 10234. | AI340582 | EST | 4077509 |
| 10235. | AI343713 | EST | 4080919 |
| 10236. | AI346427 | EST | 4083633 |
| 10237. | AI346736 | EST | 4083942 |
| 10238. | AI357472 | EST | 4109093 |
| 10239. | AI358107 | EST | 4109728 |
| 10240. | AI373635 | EST | 4153501 |
| 10241. | AI374628 | EST | 4174618 |
| 10242. | AI378301 | EST | 4188154 |
| 10243. | AI378581 | EST | 4188434 |
| 10244. | AI417745 | EST | 4261249 |
| 10245. | AI433548 | EST | 4289949 |
| 10246. | AI433797 | EST | 4291694 |
| 10247. | AI439089 | EST | 4301881 |
| 10248. | AI440293 | EST | 4281669 |
| 10249. | AI445432 | EST | 4288171 |
| 10250. | AI452485 | EST | 4307457 |
| 10251. | AI453203 | EST | 4308715 |
| 10252. | AI469038 | EST | 4331128 |
| 10253. | AI473779 | EST | 4326824 |
| 10254. | AI476478 | EST | 4329512 |
| 10255. | AI478418 | EST | 4371644 |
| 10256. | AI494568 | EST | 4395571 |
| 10257. | AI522052 | EST | 4436187 |
| 10258. | AI524007 | EST | 4438142 |
| 10259. | AI524304 | EST | 4438439 |
| 10260. | AI536688 | EST | 4450823 |
| 10261. | AI557497 | EST | 4489860 |
| 10262. | AI566742 | EST | 4525194 |
| 10263. | AI573140 | EST | 4536514 |
| 10264. | AI582177 | EST | 4568074 |
| 10265. | AI589301 | EST | 4598349 |
| 10266. | AI589889 | EST | 4598937 |
| 10267. | AI591025 | EST | 4600073 |
| 10268. | AI597808 | EST | 4606856 |
| 10269. | AI608953 | EST | 4618120 |
| 10270. | AI613073 | EST | 4622240 |
| 10271. | AI620037 | EST | 4629163 |
| 10272. | AI625109 | EST | 4650040 |
| 10273. | AI627542 | EST | 4664342 |
| 10274. | AI630369 | EST | 4681699 |
| 10275. | AI630593 | EST | 4681923 |
| 10276. | AI632113 | EST | 4683443 |
| 10277. | AI636113 | EST | 4687443 |
| 10278. | AI640202 | EST | 4703311 |
| 10279. | AI650352 | EST | 4734331 |
| 10280. | AI651195 | EST | 4735174 |
| 10281. | AI651840 | EST | 4735819 |
| 10282. | AI654672 | EST | 4738651 |
| 10283. | AI658993 | EST | 4762563 |
| 10284. | AI660656 | EST | 4764239 |
| 10285. | AI669253 | EST | 4834027 |
| 10286. | AI670064 | EST | 4834838 |
| 10287. | AI672364 | EST | 4852095 |
| 10288. | AI676178 | EST | 4876658 |
| 10289. | AI679667 | EST | 4889849 |
| 10290. | AI681372 | EST | 4891554 |
| 10291. | AI683431 | EST | 4893613 |
| 10292. | AI684170 | EST | 4895464 |
| 10293. | AI684650 | EST | 4895944 |
| 10294. | AI684654 | EST | 4895948 |
| 10295. | AI685733 | EST | 4897027 |
| 10296. | AI686567 | EST | 4897861 |
| 10297. | AI686957 | EST | 4898251 |
| 10298. | AI688725 | EST | 4900019 |
| 10299. | AI689248 | EST | 4900542 |
| 10300. | AI690042 | EST | 4901336 |
| 10301. | AI693969 | EST | 4971309 |
| 10302. | AI703412 | EST | 4991312 |
| 10303. | AI732472 | EST | 5053585 |
| 10304. | AI741778 | EST | 5110066 |
| 10305. | AI744486 | EST | 5112774 |
| 10306. | AI751039 | EST | 5129391 |
| 10307. | AI752391 | EST | 5130655 |
| 10308. | AI758445 | EST | 5152168 |
| 10309. | AI761728 | EST | 5177484 |
| 10310. | AI766305 | EST | 5232814 |
| 10311. | AI769450 | EST | 5235959 |
| 10312. | AI769941 | EST | 5236450 |
| 10313. | AI791537 | EST | 5339253 |
| 10314. | AI809013 | EST | 5395579 |
| 10315. | AI815198 | EST | 5426403 |
| 10316. | AI815380 | EST | 5430926 |
| 10317. | AI815388 | EST | 5430934 |
| 10318. | AI815806 | EST | 5431352 |
| 10319. | AI815972 | EST | 5431518 |
| 10320. | AI816249 | EST | 5431795 |
| 10321. | AI816446 | EST | 5431992 |
| 10322. | AI818955 | EST | 5438034 |
| 10323. | AI831610 | EST | 5452281 |
| 10324. | AI886016 | EST | 5591101 |
| 10325. | AI888922 | EST | 5594086 |
| 10326. | AI906328 | EST | 6496715 |
| 10327. | AI906336 | EST | 6496723 |
| 10328. | AI917587 | EST | 5637442 |
| 10329. | AI925863 | EST | 5661827 |
| 10330. | AI929111 | EST | 5665075 |
| 10331. | AI929762 | EST | 5665726 |
| 10332. | AI949077 | EST | 5741387 |
| 10333. | AI979015 | EST | 5804045 |
| 10334. | AL036165 | EST | 5927669 |
| 10335. | AL036339 | EST | 5927742 |
| 10336. | AL036446 | EST | 5405998 |
| 10337. | AL037235 | EST | 5406663 |
| 10338. | AL037638 | EST | 5406999 |
| 10339. | AL037661 | EST | 5407021 |
| 10340. | AL037712 | EST | 5407060 |
| 10341. | AL040146 | EST | 5409111 |
| 10342. | AL042027 | EST | 5421373 |
| 10343. | AL042265 | EST | 5421608 |
| 10344. | AL042324 | EST | 5421666 |
| 10345. | AL042404 | EST | 5421751 |
| 10346. | AL048026 | EST | 4728859 |
| 10347. | AL048037 | EST | 4728870 |
| 10348. | AL080049 | EST | 5435623 |
| 10349. | AL119219 | EST | 5925118 |
| 10350. | AL119859 | EST | 5925758 |
| 10351. | AL120840 | EST | 5926841 |
| 10352. | AL120964 | EST | 5926965 |
| 10353. | AL121010 | EST | 5927011 |
| 10354. | AL135297 | EST | 6603484 |
| 10355. | AL135401 | EST | 6603588 |
| 10356. | AL135582 | EST | 6603769 |
| 10357. | AL135735 | EST | 6603922 |
| 10358. | AW020109 | EST | 5873639 |
| 10359. | AW022223 | EST | 5875753 |
| 10360. | AW026359 | EST | 5879889 |
| 10361. | AW027434 | EST | 5886190 |
| 10362. | AW052046 | EST | 5914405 |
| 10363. | AW103451 | EST | 6074186 |
| 10364. | AW135761 | EST | 6139894 |
| 10365. | AW156915 | EST | 6228316 |
| 10366. | AW163632 | EST | 6302665 |
| 10367. | AW169658 | EST | 6401183 |
| 10368. | AW170193 | EST | 6401707 |
| 10369. | AW172722 | EST | 6438670 |
| 10370. | AW172972 | EST | 6438920 |
| 10371. | AW173641 | EST | 6439589 |
| 10372. | AW179011 | EST | 6445048 |
| 10373. | AW190104 | EST | 6464584 |
| 10374. | AW231970 | EST | 6564273 |
| 10375. | AW236346 | EST | 6568735 |
| 10376. | AW238475 | EST | 6570864 |
| 10377. | AW238730 | EST | 6571119 |
| 10378. | AW247691 | EST | 6590684 |
| 10379. | AW248279 | EST | 6591272 |
| 10380. | AW264516 | EST | 6641332 |
| 10381. | AW274416 | EST | 6661446 |
| 10382. | AW274631 | EST | 6661661 |
| 10383. | AW328621 | EST | 6799117 |
| 10384. | AW340834 | EST | 6837460 |
| 10385. | AW341060 | EST | 6837605 |
| 10386. | AW361325 | EST | 6865975 |
| 10387. | AW363168 | EST | 6867818 |
| 10388. | AW364389 | EST | 6869039 |
| 10389. | AW364521 | EST | 6869171 |
| 10390. | AW365954 | EST | 6870604 |
| 10391. | AW370224 | EST | 6874878 |
| 10392. | AW372807 | EST | 6877461 |
| 10393. | AW374672 | EST | 6879326 |
| 10394. | AW375944 | EST | 6880598 |
| 10395. | AW376930 | EST | 6881593 |
| 10396. | AW378191 | EST | 6882850 |
| 10397. | AW379230 | EST | 6883889 |
| 10398. | AW380311 | EST | 6884879 |
| 10399. | AW380979 | EST | 6885638 |
| 10400. | AW383328 | EST | 6887987 |
| 10401. | AW383978 | EST | 6888637 |
| 10402. | AW384930 | EST | 6889589 |
| 10403. | AW386882 | EST | 6891541 |
| 10404. | AW389868 | EST | 6894527 |
| 10405. | AW390268 | EST | 6894927 |
| 10406. | AW390747 | EST | 6895406 |
| 10407. | AW401375 | EST | 6920157 |
| 10408. | AW401500 | EST | 6920108 |
| 10409. | AW402094 | EST | 6920780 |
| 10410. | AW402940 | EST | 6921729 |
| 10411. | AW403150 | EST | 6922005 |
| 10412. | AW403588 | EST | 6922573 |
| 10413. | AW403676 | EST | 6922685 |
| 10414. | AW403935 | EST | 6922902 |
| 10415. | AW404044 | EST | 6923101 |
| 10416. | AW404099 | EST | 6923156 |
| 10417. | AW404217 | EST | 6923274 |
| 10418. | AW404734 | EST | 6923791 |
| 10419. | AW405207 | EST | 6924264 |
| 10420. | AW405840 | EST | 6924897 |
| 10421. | AW408122 | EST | 6927179 |
| 10422. | AW419014 | EST | 6946946 |
| 10423. | AW419384 | EST | 6947316 |
| 10424. | AW419457 | EST | 6947389 |
| 10425. | AW438516 | EST | 6973822 |
| 10426. | AW438867 | EST | 6974173 |
| 10427. | AW440427 | EST | 6975733 |
| 10428. | AW440655 | EST | 6975961 |
| 10429. | AW466931 | EST | 7037037 |
| 10430. | AW499520 | EST | 7111261 |
| 10431. | AW503073 | EST | 7118135 |
| 10432. | AW511241 | EST | 7149319 |
| 10433. | AW512447 | EST | 7150525 |
| 10434. | AW575067 | EST | 7246606 |
| 10435. | AW580040 | EST | 7255089 |
| 10436. | AW580650 | EST | 7255699 |
| 10437. | AW581990 | EST | 7257039 |
| 10438. | AW951149 | EST | 8140816 |
| 10439. | D51079 | EST | 950832 |
| 10440. | D53396 | EST | 955293 |
| 10441. | D54275 | EST | 956172 |
| 10442. | D56472 | EST | 971087 |
| 10443. | D58694 | EST | 968328 |
| 10444. | D59491 | EST | 960597 |
| 10445. | D80228 | EST | 1178105 |
| 10446. | D81727 | EST | 1180358 |
| 10447. | F07635 | EST | 673321 |
| 10448. | F08552 | EST | 677119 |
| 10449. | H00742 | EST | 863675 |
| 10450. | H02470 | EST | 865403 |
| 10451. | H02725 | EST | 865658 |
| 10452. | H15671 | EST | 880491 |
| 10453. | H16423 | EST | 881243 |
| 10454. | H25505 | EST | 894628 |
| 10455. | H38073 | EST | 907572 |
| 10456. | H39777 | EST | 915829 |
| 10457. | H82745 | EST | 1060834 |
| 10458. | N20605 | EST | 1125560 |
| 10459. | N26942 | EST | 1141290 |
| 10460. | N27716 | EST | 1142197 |
| 10461. | N28466 | EST | 1146702 |
| 10462. | N36819 | EST | 1157961 |
| 10463. | N43012 | EST | 1166756 |
| 10464. | N44316 | EST | 1182834 |
| 10465. | N47378 | EST | 1188544 |
| 10466. | N62491 | EST | 1210320 |
| 10467. | N79738 | EST | 1242439 |
| 10468. | N94740 | EST | 1267029 |
| 10469. | R13437 | EST | 766513 |
| 10470. | R41605 | EST | 799829 |
| 10471. | R53758 | EST | 815660 |
| 10472. | R69737 | EST | 843254 |
| 10473. | T05916 | EST | 317066 |
| 10474. | T25412 | EST | 2947349 |
| 10475. | T50226 | EST | 652086 |
| 10476. | T64797 | EST | 673842 |
| 10477. | T81910 | EST | 704917 |
| 10478. | W01279 | EST | 1273259 |
| 10479. | W01739 | EST | 1273719 |
| 10480. | W01801 | EST | 1274002 |
| 10481. | W03533 | EST | 1275408 |
| 10482. | W04336 | EST | 1276235 |
| 10483. | W07169 | EST | 1281180 |
| 10484. | W15195 | EST | 1289576 |
| 10485. | W19723 | EST | 1295622 |
| 10486. | W28518 | EST | 1308466 |
| 10487. | W38691 | EST | 1320417 |
| 10488. | W47066 | EST | 1331705 |
| 10489. | W55981 | EST | 1357870 |
| 10490. | W72967 | EST | 1383110 |
| 10491. | W73765 | EST | 1383910 |
| 10492. | W76249 | EST | 1386631 |
| 10493. | Z19286 | EST | 29115 |
| 10494. | Z30248 | EST | 456628 |
| 10495. | Z45496 | EST | 574710 |
| 10496. | A16030 | NUCPATENT | 492040 |
| 10497. | T59274 | NUCPATENT | 661111 |
| 10498. | V04699 | NUCPATENT | N/A |
| 10499. | V05728 | NUCPATENT | N/A |
| 10500. | V24133 | NUCPATENT | N/A |
| 10501. | V34185 | NUCPATENT | N/A |
| 10502. | V35457 | NUCPATENT | N/A |
| 10503. | V69700 | NUCPATENT | N/A |
| 10504. | X04354 | NUCPATENT | 60995 |
| 10505. | X24011 | NUCPATENT | N/A |
| 10506. | X40197 | NUCPATENT | N/A |
| 10507. | X59868 | NUCPATENT | 50357 |
| 10508. | X89854 | NUCPATENT | 927383 |
| 10509. | X98008 | NUCPATENT | 1360601 |
| 10510. | Z15187 | NUCRATENT | 288856 |
| 10511. | Z18356 | NUCPATENT | 30768 |
| 10512. | Z23888 | NUCPATENT | 394088 |
| 10513. | Z24392 | NUCPATENT | 394592 |
| 10514. | Z28693 | NUCPATENT | 434350 |
| 10515. | Z29299 | NUCPATENT | 440480 |
| 10516. | Z33584 | NUCPATENT | 488673 |
| 10517. | Z50012 | NUCPATENT | 1297046 |
| 10518. | Z50890 | NUCPATENT | 1232190 |
| 10519. | Z51355 | NUCPATENT | 1232655 |
| 10520. | Z52474 | NUCPATENT | 1233774 |
| 10521. | Z58233 | NUCPATENT | 1029464 |
| 10522. | Z58568 | NUCPATENT | 1029799 |
| 10523. | Z97238 | NUCPATENT | 2245218 |
| 10524. | AC31942 | PREPATNUC | N/A |
| 10525. | AC39892 | PREPATNUC | N/A |
| 10526. | AC41861 | PREPATNUC | N/A |
| 10527. | AC42476 | PREPATNUC | N/A |
| 10528. | AC42921 | PREPATNUC | N/A |
| 10529. | A12178 | Genbank | 490111 |
| 10530. | A14133 | Genbank | 490127 |
| 10531. | A26221 | Genbank | 904883 |
| 10532. | AB000888 | Genbank | 2467297 |
| 10533. | AB002374 | Genbank | 2280484 |
| 10534. | AB002380 | Genbank | 2224704 |
| 10535. | AB006651 | Genbank | 3201570 |
| 10536. | AB007885 | Genbank | 2887424 |
| 10537. | AB007893 | Genbank | 2887436 |
| 10538. | AB007916 | Genbank | 6683704 |
| 10539. | AB007922 | Genbank | 6634036 |
| 10540. | AB008109 | Genbank | 2554613 |
| 10541. | AB011145 | Genbank | 3043669 |
| 10542. | AB014575 | Genbank | 3327163 |
| 10543. | AB014586 | Genbank | 3327185 |
| 10544. | AB015317 | Genbank | 3641673 |
| 10545. | AB016207 | Genbank | 3721865 |
| 10546. | AB018272 | Genbank | 3882178 |
| 10547. | AB019038 | Genbank | 6970469 |
| 10548. | AB019564 | Genbank | 3885367 |
| 10549. | AB019568 | Genbank | 3885371 |
| 10550. | AB020631 | Genbank | 4240136 |
| 10551. | AB020634 | Genbank | 4240142 |
| 10552. | AB020656 | Genbank | 4240186 |
| 10553. | AB020878 | Genbank | 4003398 |
| 10554. | AB020980 | Genbank | 6467174 |
| 10555. | AB022537 | Genbank | 6942217 |
| 10556. | AB023212 | Genbank | 4589633 |
| 10557. | AB024057 | Genbank | 4514652 |
| 10558. | AB028624 | Genbank | 5103045 |
| 10559. | AB028948 | Genbank | 5689386 |
| 10560. | AB029290 | Genbank | 5821433 |
| 10561. | AB030251 | Genbank | 6759254 |
| 10562. | AB032251 | Genbank | 6683491 |
| 10563. | AB032255 | Genbank | 6683499 |
| 10564. | AB033033 | Genbank | 6330496 |
| 10565. | AB033045 | Genbank | 6330589 |
| 10566. | AB033070 | Genbank | 6330818 |
| 10567. | AB033082 | Genbank | 6330910 |
| 10568. | AB037737 | Genbank | 7243012 |
| 10569. | AB037790 | Genbank | 7243118 |
| 10570. | AB037835 | Genbank | 7243208 |
| 10571. | AB037841 | Genbank | 7243220 |
| 10572. | AC000040 | Genbank | 7923866 |
| 10573. | AC000120 | Genbank | 1809224 |
| 10574. | AC000353 | Genbank | 6970735 |
| 10575. | AC000397 | Genbank | 2133896 |
| 10576. | AC001228 | Genbank | 1935053 |
| 10577. | AC001234 | Genbank | 4028939 |
| 10578. | AC002038 | Genbank | 2226439 |
| 10579. | AC002045 | Genbank | 2951945 |
| 10580. | AC002051 | Genbank | 7798752 |
| 10581. | AC002064 | Genbank | 2076723 |
| 10582. | AC002067 | Genbank | 2076720 |
| 10583. | AC002074 | Genbank | 2078467 |
| 10584. | AC002094 | Genbank | 2155224 |
| 10585. | AC002096 | Genbank | 2599243 |
| 10586. | AC002326 | Genbank | 2288970 |
| 10587. | AC002366 | Genbank | 2739349 |
| 10588. | AC002404 | Genbank | 2329922 |
| 10589. | AC002449 | Genbank | 2337886 |
| 10590. | AC002451 | Genbank | 2337882 |
| 10591. | AC002459 | Genbank | 2337870 |
| 10592. | AC002487 | Genbank | 2341013 |
| 10593. | AC002519 | Genbank | 2815551 |
| 10594. | AC002531 | Genbank | 2580476 |
| 10595. | AC002549 | Genbank | 2992475 |
| 10596. | AC002559 | Genbank | 3868737 |
| 10597. | AC002565 | Genbank | 2896804 |
| 10598. | AC002978 | Genbank | 2961441 |
| 10599. | AC003007 | Genbank | 2911728 |
| 10600. | AC003072 | Genbank | 2588642 |
| 10601. | AC003964 | Genbank | 2981253 |
| 10602. | AC003989 | Genbank | 2772538 |
| 10603. | AC003991 | Genbank | 2772535 |
| 10604. | AC004033 | Genbank | 4581183 |
| 10605. | AC004051 | Genbank | 3367539 |
| 10606. | AC004061 | Genbank | 3108028 |
| 10607. | AC004066 | Genbank | 4001527 |
| 10608. | AC004084 | Genbank | 2822156 |
| 10609. | AC004098 | Genbank | 3097872 |
| 10610. | AC004099 | Genbank | 3650064 |
| 10611. | AC004112 | Genbank | 2828785 |
| 10612. | AC004142 | Genbank | 2880078 |
| 10613. | AC004230 | Genbank | 4028938 |
| 10614. | AC004236 | Genbank | 2914668 |
| 10615. | AC004459 | Genbank | 2979594 |
| 10616. | AC004460 | Genbank | 2981263 |
| 10617. | AC004500 | Genbank | 2996639 |
| 10618. | AC004520 | Genbank | 3004572 |
| 10619. | AC004527 | Genbank | 4760422 |
| 10620. | AC004549 | Genbank | 3046306 |
| 10621. | AC004582 | Genbank | 3337307 |
| 10622. | AC004587 | Genbank | 3150014 |
| 10623. | AC004594 | Genbank | 3063516 |
| 10624. | AC004605 | Genbank | 3337395 |
| 10625. | AC004626 | Genbank | 3337396 |
| 10626. | AC004677 | Genbank | 3126881 |
| 10627. | AC004686 | Genbank | 3688105 |
| 10628. | AC004703 | Genbank | 3228509 |
| 10629. | AC004741 | Genbank | 3152631 |
| 10630. | AC004796 | Genbank | 4454488 |
| 10631. | AC004800 | Genbank | 3184503 |
| 10632. | AC004804 | Genbank | 3406032 |
| 10633. | AC004814 | Genbank | 5708498 |
| 10634. | AC004890 | Genbank | 4508146 |
| 10635. | AC004902 | Genbank | 5757546 |
| 10636. | AC004904 | Genbank | 4156180 |
| 10637. | AC004918 | Genbank | 4153871 |
| 10638. | AC004972 | Genbank | 4926904 |
| 10639. | AC004986 | Genbank | 4309812 |
| 10640. | AC004999 | Genbank | 3970963 |
| 10641. | AC005007 | Genbank | 4156151 |
| 10642. | AC005031 | Genbank | 3688109 |
| 10643. | AC005037 | Genbank | 4827310 |
| 10644. | AC005050 | Genbank | 5757513 |
| 10645. | AC005062 | Genbank | 4150931 |
| 10646. | AC005065 | Genbank | 4153861 |
| 10647. | AC005069 | Genbank | 4753224 |
| 10648. | AC005074 | Genbank | 4153859 |
| 10649. | AC005083 | Genbank | 4150930 |
| 10650. | AC005084 | Genbank | 3659503 |
| 10651. | AC005166 | Genbank | 3242769 |
| 10652. | AC005221 | Genbank | 3282158 |
| 10653. | AC005226 | Genbank | 3970969 |
| 10654. | AC005239 | Genbank | 3287673 |
| 10655. | AC005524 | Genbank | 3451031 |
| 10656. | AC005550 | Genbank | 3478668 |
| 10657. | AC005630 | Genbank | 4159882 |
| 10658. | AC005692 | Genbank | 4159889 |
| 10659. | AC005696 | Genbank | 3819097 |
| 10660. | AC005740 | Genbank | 3687210 |
| 10661. | AC005746 | Genbank | 3808088 |
| 10662. | AC005787 | Genbank | 3702285 |
| 10663. | AC005796 | Genbank | 3702268 |
| 10664. | AC005829 | Genbank | 3873300 |
| 10665. | AC005838 | Genbank | 3947427 |
| 10666. | AC005856 | Genbank | 3849821 |
| 10667. | AC005875 | Genbank | 6513908 |
| 10668. | AC005876 | Genbank | 6249672 |
| 10669. | AC005905 | Genbank | 4090182 |
| 10670. | AC005921 | Genbank | 4982556 |
| 10671. | AC006021 | Genbank | 4731073 |
| 10672. | AC006031 | Genbank | 4309811 |
| 10673. | AC006033 | Genbank | 4309948 |
| 10674. | AC006040 | Genbank | 10801470 |
| 10675. | AC006059 | Genbank | 4544348 |
| 10676. | AC006079 | Genbank | 4006838 |
| 10677. | AC006084 | Genbank | 3953485 |
| 10678. | AC006130 | Genbank | 3970932 |
| 10679. | AC006146 | Genbank | 5836157 |
| 10680. | AC006237 | Genbank | 4062175 |
| 10681. | AC006238 | Genbank | 4204704 |
| 10682. | AC006312 | Genbank | 4582483 |
| 10683. | AC006335 | Genbank | 4753229 |
| 10684. | AC006352 | Genbank | 4753268 |
| 10685. | AC006359 | Genbank | 4753263 |
| 10686. | AC006462 | Genbank | 10801468 |
| 10687. | AC006474 | Genbank | 4454627 |
| 10688. | AC006475 | Genbank | 4753283 |
| 10689. | AC006501 | Genbank | 4309874 |
| 10690. | AC006548 | Genbank | 7109503 |
| 10691. | AC006840 | Genbank | 6227027 |
| 10692. | AC006977 | Genbank | 5931479 |
| 10693. | AC006978 | Genbank | 4753257 |
| 10694. | AC006987 | Genbank | 5123989 |
| 10695. | AC006991 | Genbank | 10801457 |
| 10696. | AC007009 | Genbank | 7322010 |
| 10697. | AC007014 | Genbank | 4371263 |
| 10698. | AC007016 | Genbank | 4760420 |
| 10699. | AC007032 | Genbank | 5523832 |
| 10700. | AC007036 | Genbank | 5649379 |
| 10701. | AC007040 | Genbank | 5306302 |
| 10702. | AC007052 | Genbank | 4510438 |
| 10703. | AC007276 | Genbank | 6587937 |
| 10704. | AC007308 | Genbank | 5903110 |
| 10705. | AC007347 | Genbank | 6806840 |
| 10706. | AC007372 | Genbank | 4927301 |
| 10707. | AC007384 | Genbank | 5708473 |
| 10708. | AC007390 | Genbank | 6358845 |
| 10709. | AC007393 | Genbank | 5306300 |
| 10710. | AC007533 | Genbank | 6682595 |
| 10711. | AC007684 | Genbank | 5836173 |
| 10712. | AC007695 | Genbank | 5815500 |
| 10713. | AC007878 | Genbank | 5732161 |
| 10714. | AC007899 | Genbank | 6289213 |
| 10715. | AC008014 | Genbank | 5815493 |
| 10716. | AC008039 | Genbank | 5454236 |
| 10717. | AC008040 | Genbank | 5922025 |
| 10718. | AC008070 | Genbank | 8748906 |
| 10719. | AC008071 | Genbank | 5836182 |
| 10720. | AC008078 | Genbank | 6006920 |
| 10721. | AC008079 | Genbank | 7940363 |
| 10722. | AC008123 | Genbank | 6006046 |
| 10723. | AC008151 | Genbank | 5629925 |
| 10724. | AC008180 | Genbank | 9558559 |
| 10725. | AC008269 | Genbank | 10716637 |
| 10726. | AC008553 | Genbank | 7670114 |
| 10727. | AC008585 | Genbank | 6721144 |
| 10728. | AC008772 | Genbank | 8844106 |
| 10729. | AC008934 | Genbank | 7158896 |
| 10730. | AC009181 | Genbank | 6692751 |
| 10731. | AC009230 | Genbank | 7408128 |
| 10732. | AC009235 | Genbank | 9454633 |
| 10733. | AC009303 | Genbank | 10716638 |
| 10734. | AC009318 | Genbank | 7656679 |
| 10735. | AC009478 | Genbank | 7770675 |
| 10736. | AC009958 | Genbank | 7408124 |
| 10737. | AC010168 | Genbank | 6855156 |
| 10738. | AC010202 | Genbank | 6598666 |
| 10739. | AC010382 | Genbank | 6850297 |
| 10740. | AC011331 | Genbank | 9929686 |
| 10741. | AC011592 | Genbank | 6514033 |
| 10742. | AC012087 | Genbank | 7114465 |
| 10743. | AC016681 | Genbank | 7321924 |
| 10744. | AC018641 | Genbank | 8705046 |
| 10745. | AC018816 | Genbank | 9755439 |
| 10746. | AC019106 | Genbank | 8570227 |
| 10747. | AC019181 | Genbank | 8748861 |
| 10748. | AC020633 | Genbank | 7658301 |
| 10749. | AC020728 | Genbank | 7770044 |
| 10750. | AC022493 | Genbank | 9558552 |
| 10751. | AC024083 | Genbank | 9789634 |
| 10752. | AC027663 | Genbank | 8698752 |
| 10753. | AF001549 | Genbank | 3355302 |
| 10754. | AF006070 | Genbank | 3309104 |
| 10755. | AF007544 | Genbank | 2970122 |
| 10756. | AF010313 | Genbank | 6468761 |
| 10757. | AF012072 | Genbank | 9967556 |
| 10758. | AF013758 | Genbank | 3046899 |
| 10759. | AF014402 | Genbank | 3123847 |
| 10760. | AF014882 | Genbank | 2582056 |
| 10761. | AF015812 | Genbank | 2599359 |
| 10762. | AF016507 | Genbank | 2909776 |
| 10763. | AF020202 | Genbank | 2431999 |
| 10764. | AF023268 | Genbank | 2564910 |
| 10765. | AF026445 | Genbank | 2739086 |
| 10766. | AF028832 | Genbank | 3287488 |
| 10767. | AF029689 | Genbank | 4545074 |
| 10768. | AF032922 | Genbank | 3820481 |
| 10769. | AF035429 | Genbank | 2665724 |
| 10770. | AF038458 | Genbank | 2689079 |
| 10771. | AF042857 | Genbank | 2952271 |
| 10772. | AF047020 | Genbank | 4204096 |
| 10773. | AF050641 | Genbank | 5326822 |
| 10774. | AF052113 | Genbank | 3360420 |
| 10775. | AF054990 | Genbank | 3005703 |
| 10776. | AF060981 | Genbank | 3851501 |
| 10777. | AF061258 | Genbank | 3108092 |
| 10778. | AF061737 | Genbank | 4335938 |
| 10779. | AF064867 | Genbank | 3800768 |
| 10780. | AF070540 | Genbank | 3387898 |
| 10781. | AF070649 | Genbank | 3283923 |
| 10782. | AF077052 | Genbank | 4689151 |
| 10783. | AF077202 | Genbank | 4679017 |
| 10784. | AF077951 | Genbank | 3643106 |
| 10785. | AF078849 | Genbank | 5531812 |
| 10786. | AF078860 | Genbank | 5531834 |
| 10787. | AF080092 | Genbank | 4322311 |
| 10788. | AF086311 | Genbank | 3483656 |
| 10789. | AF086735 | Genbank | 3859945 |
| 10790. | AF087659 | Genbank | 4191345 |
| 10791. | AF088055 | Genbank | 3523261 |
| 10792. | AF090884 | Genbank | 6690150 |
| 10793. | AF090928 | Genbank | 6690222 |
| 10794. | AF090935 | Genbank | 6690235 |
| 10795. | AF091214 | Genbank | 3719420 |
| 10796. | AF091433 | Genbank | 3769613 |
| 10797. | AF092907 | Genbank | 4210952 |
| 10798. | AF093097 | Genbank | 6002622 |
| 10799. | AF100620 | Genbank | 4808630 |
| 10800. | AF103907 | Genbank | 6165973 |
| 10801. | AF103908 | Genbank | 6165974 |
| 10802. | AF106943 | Genbank | 4454994 |
| 10803. | AF107885 | Genbank | 3928926 |
| 10804. | AF110368 | Genbank | 6502538 |
| 10805. | AF111345 | Genbank | 4731238 |
| 10806. | AF113016 | Genbank | 6642755 |
| 10807. | AF113699 | Genbank | 6855632 |
| 10808. | AF113702 | Genbank | 6855636 |
| 10809. | AF117229 | Genbank | 6563231 |
| 10810. | AF117829 | Genbank | 4151947 |
| 10811. | AF121202 | Genbank | 6572527 |
| 10812. | AF123534 | Genbank | 4680297 |
| 10813. | AF127577 | Genbank | 7019596 |
| 10814. | AF129076 | Genbank | 4325383 |
| 10815. | AF131215 | Genbank | 8272457 |
| 10816. | AF131738 | Genbank | 4406548 |
| 10817. | AF131742 | Genbank | 4406554 |
| 10818. | AF131750 | Genbank | 4406566 |
| 10819. | AF132952 | Genbank | 4680674 |
| 10820. | AF135162 | Genbank | 7259481 |
| 10821. | AF142063 | Genbank | 5456839 |
| 10822. | AF146367 | Genbank | 4877991 |
| 10823. | AF147331 | Genbank | 4761682 |
| 10824. | AF147394 | Genbank | 4761745 |
| 10825. | AF148461 | Genbank | 5002304 |
| 10826. | AF150735 | Genbank | 6690509 |
| 10827. | AF151063 | Genbank | 7106847 |
| 10828. | AF151103 | Genbank | 5758136 |
| 10829. | AF153609 | Genbank | 5231142 |
| 10830. | AF161383 | Genbank | 6841179 |
| 10831. | AF161384 | Genbank | 6841181 |
| 10832. | AF161419 | Genbank | 6841251 |
| 10833. | AF161458 | Genbank | 6841439 |
| 10834. | AF161800 | Genbank | 5353769 |
| 10835. | AF164609 | Genbank | 5802809 |
| 10836. | AF176555 | Genbank | 6318854 |
| 10837. | AF176574 | Genbank | 5762481 |
| 10838. | AF182289 | Genbank | 5919146 |
| 10839. | AF187554 | Genbank | 6653225 |
| 10840. | AF192043 | Genbank | 7141064 |
| 10841. | AF203815 | Genbank | 6979641 |
| 10842. | AF223408 | Genbank | 6911266 |
| 10843. | AF224669 | Genbank | 7012905 |
| 10844. | AF240627 | Genbank | 7263183 |
| 10845. | AF250841 | Genbank | 8896064 |
| 10846. | AJ000332 | Genbank | 2274967 |
| 10847. | AJ001382 | Genbank | 2764618 |
| 10848. | AJ005273 | Genbank | 3850703 |
| 10849. | AJ009870 | Genbank | 4218430 |
| 10850. | AJ225782 | Genbank | 4165269 |
| 10851. | AJ227870 | Genbank | 3183908 |
| 10852. | AJ250075 | Genbank | 6013007 |
| 10853. | AJ250915 | Genbank | 6996445 |
| 10854. | AJ400877 | Genbank | 8052236 |
| 10855. | AK000115 | Genbank | 7019992 |
| 10856. | AK000427 | Genbank | 7020507 |
| 10857. | AK000447 | Genbank | 7020542 |
| 10858. | AK000509 | Genbank | 7020648 |
| 10859. | AK000540 | Genbank | 7020703 |
| 10860. | AK000548 | Genbank | 7020717 |
| 10861. | AK000571 | Genbank | 7020756 |
| 10862. | AK000624 | Genbank | 7020839 |
| 10863. | AK000712 | Genbank | 7020971 |
| 10864. | AK000715 | Genbank | 7020977 |
| 10865. | AK000726 | Genbank | 7020992 |
| 10866. | AK000820 | Genbank | 7021131 |
| 10867. | AK000869 | Genbank | 7021195 |
| 10868. | AK001054 | Genbank | 7022084 |
| 10869. | AK001075 | Genbank | 7022117 |
| 10870. | AK001253 | Genbank | 7022393 |
| 10871. | AK001293 | Genbank | 7022457 |
| 10872. | AK001429 | Genbank | 7022680 |
| 10873. | AK001461 | Genbank | 7022732 |
| 10874. | AK001559 | Genbank | 7022884 |
| 10875. | AK001662 | Genbank | 7023057 |
| 10876. | AK001667 | Genbank | 7023064 |
| 10877. | AK001741 | Genbank | 7023192 |
| 10878. | AK001886 | Genbank | 7023430 |
| 10879. | AK001948 | Genbank | 7023529 |
| 10880. | AK002088 | Genbank | 7023760 |
| 10881. | AL020990 | Genbank | 3980348 |
| 10882. | AL021368 | Genbank | 3080468 |
| 10883. | AL021579 | Genbank | 4375984 |
| 10884. | AL021878 | Genbank | 3204432 |
| 10885. | AL021939 | Genbank | 3135969 |
| 10886. | AL022476 | Genbank | 4493520 |
| 10887. | AL023281 | Genbank | 3449132 |
| 10888. | AL023798 | Genbank | 3550031 |
| 10889. | AL023803 | Genbank | 9408726 |
| 10890. | AL024508 | Genbank | 3445456 |
| 10891. | AL031003 | Genbank | 4007185 |
| 10892. | AL031259 | Genbank | 3790132 |
| 10893. | AL031277 | Genbank | 4375907 |
| 10894. | AL031589 | Genbank | 4914506 |
| 10895. | AL031652 | Genbank | 10198596 |
| 10896. | AL031669 | Genbank | 8218059 |
| 10897. | AL031777 | Genbank | 10198609 |
| 10898. | AL031780 | Genbank | 4140349 |
| 10899. | AL031781 | Genbank | 4038570 |
| 10900. | AL033531 | Genbank | 6807582 |
| 10901. | AL034343 | Genbank | 5931807 |
| 10902. | AL034375 | Genbank | 5763835 |
| 10903. | AL034380 | Genbank | 7981257 |
| 10904. | AL034417 | Genbank | 5102616 |
| 10905. | AL034421 | Genbank | 10198621 |
| 10906. | AL034553 | Genbank | 5419653 |
| 10907. | AL035071 | Genbank | 5002606 |
| 10908. | AL035209 | Genbank | 4160217 |
| 10909. | AL035420 | Genbank | 5918161 |
| 10910. | AL035457 | Genbank | 6143575 |
| 10911. | AL035530 | Genbank | 5918320 |
| 10912. | AL035563 | Genbank | 5360972 |
| 10913. | AL035633 | Genbank | 6580404 |
| 10914. | AL049367 | Genbank | 4500158 |
| 10915. | AL049381 | Genbank | 4500168 |
| 10916. | AL049564 | Genbank | 4902757 |
| 10917. | AL049697 | Genbank | 6456830 |
| 10918. | AL049742 | Genbank | 5419783 |
| 10919. | AL049766 | Genbank | 5763746 |
| 10920. | AL049779 | Genbank | 8176894 |
| 10921. | AL049782 | Genbank | 4902604 |
| 10922. | AL049783 | Genbank | 4902605 |
| 10923. | AL049797 | Genbank | 5419825 |
| 10924. | AL049834 | Genbank | 6634060 |
| 10925. | AL049859 | Genbank | 5706480 |
| 10926. | AL049911 | Genbank | 6114591 |
| 10927. | AL050082 | Genbank | 4884316 |
| 10928. | AL050141 | Genbank | 4884352 |
| 10929. | AL050144 | Genbank | 4884151 |
| 10930. | AL050273 | Genbank | 4886450 |
| 10931. | AL050290 | Genbank | 4886512 |
| 10932. | AL050302 | Genbank | 6522975 |
| 10933. | AL050341 | Genbank | 5791509 |
| 10934. | AL050366 | Genbank | 4914599 |
| 10935. | AL078583 | Genbank | 5805131 |
| 10936. | AL078612 | Genbank | 5419788 |
| 10937. | AL079298 | Genbank | 5102736 |
| 10938. | AL080202 | Genbank | 5262687 |
| 10939. | AL080250 | Genbank | 5804917 |
| 10940. | AL080276 | Genbank | 5763753 |
| 10941. | AL109613 | Genbank | 6136931 |
| 10942. | AL109758 | Genbank | 8217865 |
| 10943. | AL109923 | Genbank | 8894632 |
| 10944. | AL110185 | Genbank | 5817098 |
| 10945. | AL110252 | Genbank | 5817211 |
| 10946. | AL117339 | Genbank | 6624931 |
| 10947. | AL117341 | Genbank | 7327972 |
| 10948. | AL117350 | Genbank | 7159817 |
| 10949. | AL117353 | Genbank | 6165379 |
| 10950. | AL117423 | Genbank | 5911854 |
| 10951. | AL117441 | Genbank | 5911883 |
| 10952. | AL117596 | Genbank | 5912164 |
| 10953. | AL121735 | Genbank | 6012990 |
| 10954. | AL121748 | Genbank | 6624930 |
| 10955. | AL121790 | Genbank | 8919824 |
| 10956. | AL121909 | Genbank | 8218087 |
| 10957. | AL121913 | Genbank | 7161781 |
| 10958. | AL121963 | Genbank | 7161783 |
| 10959. | AL121993 | Genbank | 8218080 |
| 10960. | AL122062 | Genbank | 6102854 |
| 10961. | AL122072 | Genbank | 6102870 |
| 10962. | AL132656 | Genbank | 8439394 |
| 10963. | AL132777 | Genbank | 6562026 |
| 10964. | AL132855 | Genbank | 7019733 |
| 10965. | AL133084 | Genbank | 6453532 |
| 10966. | AL133216 | Genbank | 6983473 |
| 10967. | AL133227 | Genbank | 9187135 |
| 10968. | AL133255 | Genbank | 8894156 |
| 10969. | AL133282 | Genbank | 8246854 |
| 10970. | AL133367 | Genbank | 7708222 |
| 10971. | AL133555 | Genbank | 6599122 |
| 10972. | AL135744 | Genbank | 6682291 |
| 10973. | AL135940 | Genbank | 7076431 |
| 10974. | AL136097 | Genbank | 7799632 |
| 10975. | AL136125 | Genbank | 9187152 |
| 10976. | AL136543 | Genbank | 6807646 |
| 10977. | AL136593 | Genbank | 7018431 |
| 10978. | AL137067 | Genbank | 8452467 |
| 10979. | AL137228 | Genbank | 7263862 |
| 10980. | AL137681 | Genbank | 6807931 |
| 10981. | AL137818 | Genbank | 7009598 |
| 10982. | AL138758 | Genbank | 8670587 |
| 10983. | AL157440 | Genbank | 7018515 |
| 10984. | AL157879 | Genbank | 8217606 |
| 10985. | AL157952 | Genbank | 8894241 |
| 10986. | AL161670 | Genbank | 7523421 |
| 10987. | AL163202 | Genbank | 7717242 |
| 10988. | AL163285 | Genbank | 7717384 |
| 10989. | AP000009 | Genbank | 4666255 |
| 10990. | AP000022 | Genbank | 4666266 |
| 10991. | AP000024 | Genbank | 3132334 |
| 10992. | AP000031 | Genbank | 3132341 |
| 10993. | AP000052 | Genbank | 3132362 |
| 10994. | AP000057 | Genbank | 3133144 |
| 10995. | AP000131 | Genbank | 4730900 |
| 10996. | AP000216 | Genbank | 4827262 |
| 10997. | AP000217 | Genbank | 4827264 |
| 10998. | AP000345 | Genbank | 5103008 |
| 10999. | AP000352 | Genbank | 6016843 |
| 11000. | AP000426 | Genbank | 8698836 |
| 11001. | AP000431 | Genbank | 7262567 |
| 11002. | AP000493 | Genbank | 5926660 |
| 11003. | AP000528 | Genbank | 5931506 |
| 11004. | AP000529 | Genbank | 5931507 |
| 11005. | AP000533 | Genbank | 5931511 |
| 11006. | AP000534 | Genbank | 5931512 |
| 11007. | AP000535 | Genbank | 5931513 |
| 11008. | AP000547 | Genbank | 5931525 |
| 11009. | AP000697 | Genbank | 6712194 |
| 11010. | AP001035 | Genbank | 6693585 |
| 11011. | AP001036 | Genbank | 6693586 |
| 11012. | AP001037 | Genbank | 6693587 |
| 11013. | AP001172 | Genbank | 6983884 |
| 11014. | AP001330 | Genbank | 8698845 |
| 11015. | D00759 | Genbank | 220021 |
| 11016. | D10040 | Genbank | 219899 |
| 11017. | D10704 | Genbank | 219540 |
| 11018. | D13388 | Genbank | 219587 |
| 11019. | D13629 | Genbank | 285984 |
| 11020. | D14710 | Genbank | 559324 |
| 11021. | D21209 | Genbank | 452189 |
| 11022. | D25542 | Genbank | 662389 |
| 11023. | D26070 | Genbank | 559322 |
| 11024. | D29954 | Genbank | 473940 |
| 11025. | D29956 | Genbank | 473944 |
| 11026. | D38112 | Genbank | 644480 |
| 11027. | D43717 | Genbank | 600558 |
| 11028. | D44466 | Genbank | 1808577 |
| 11029. | D45198 | Genbank | 971271 |
| 11030. | D63861 | Genbank | 1769811 |
| 11031. | D63998 | Genbank | 974733 |
| 11032. | D64015 | Genbank | 2281005 |
| 11033. | D80009 | Genbank | 1136433 |
| 11034. | D84064 | Genbank | 2618587 |
| 11035. | D84294 | Genbank | 1632761 |
| 11036. | D86985 | Genbank | 6634002 |
| 11037. | D87438 | Genbank | 2055294 |
| 11038. | D87666 | Genbank | 1620016 |
| 11039. | D89667 | Genbank | 1731808 |
| 11040. | D89675 | Genbank | 2055308 |
| 11041. | D90359 | Genbank | 559319 |
| 11042. | E01094 | Genbank | 2169353 |
| 11043. | E02628 | Genbank | 2170856 |
| 11044. | E06721 | Genbank | 2174903 |
| 11045. | E13124 | Genbank | 3251936 |
| 11046. | J01415 | Genbank | 1944628 |
| 11047. | J02846 | Genbank | 339505 |
| 11048. | J03779 | Genbank | 179833 |
| 11049. | J03934 | Genbank | 189245 |
| 11050. | J04177 | Genbank | 179729 |
| 11051. | J04443 | Genbank | 551175 |
| 11052. | J04615 | Genbank | 338246 |
| 11053. | J04988 | Genbank | 184422 |
| 11054. | K00422 | Genbank | 184322 |
| 11055. | K01911 | Genbank | 189273 |
| 11056. | K03432 | Genbank | 337377 |
| 11057. | L00160 | Genbank | 189904 |
| 11058. | L00352 | Genbank | 187094 |
| 11059. | L04284 | Genbank | 184393 |
| 11060. | L05091 | Genbank | 388030 |
| 11061. | L05093 | Genbank | 401844 |
| 11062. | L12168 | Genbank | 178083 |
| 11063. | L22009 | Genbank | 347313 |
| 11064. | L29050 | Genbank | 517061 |
| 11065. | L29496 | Genbank | 460280 |
| 11066. | L32592 | Genbank | 1723157 |
| 11067. | L34840 | Genbank | 2766555 |
| 11068. | L38951 | Genbank | 893287 |
| 11069. | L40521 | Genbank | 704413 |
| 11070. | L43509 | Genbank | 896282 |
| 11071. | M10119 | Genbank | 182517 |
| 11072. | M11560 | Genbank | 178350 |
| 11073. | M13692 | Genbank | 178256 |
| 11074. | M16553 | Genbank | 339503 |
| 11075. | M16768 | Genbank | 339399 |
| 11076. | M17886 | Genbank | 190233 |
| 11077. | M18157 | Genbank | 186640 |
| 11078. | M19283 | Genbank | 178042 |
| 11079. | M20132 | Genbank | 178627 |
| 11080. | M22382 | Genbank | 190126 |
| 11081. | M22430 | Genbank | 190888 |
| 11082. | M23077 | Genbank | 189830 |
| 11083. | M23483 | Genbank | 186996 |
| 11084. | M24486 | Genbank | 190785 |
| 11085. | M24545 | Genbank | 187434 |
| 11086. | M24902 | Genbank | 189618 |
| 11087. | M26325 | Genbank | 186688 |
| 11088. | M26481 | Genbank | 619789 |
| 11089. | M27024 | Genbank | 341598 |
| 11090. | M27507 | Genbank | 179400 |
| 11091. | M28211 | Genbank | 550067 |
| 11092. | M29064 | Genbank | 337452 |
| 11093. | M29065 | Genbank | 337448 |
| 11094. | M34661 | Genbank | 184314 |
| 11095. | M34840 | Genbank | 189620 |
| 11096. | M36693 | Genbank | 338285 |
| 11097. | M55409 | Genbank | 189596 |
| 11098. | M64098 | Genbank | 183891 |
| 11099. | M64571 | Genbank | 187382 |
| 11100. | M72709 | Genbank | 179073 |
| 11101. | M74002 | Genbank | 178996 |
| 11102. | M83822 | Genbank | 1580780 |
| 11103. | M85164 | Genbank | 338034 |
| 11104. | M95571 | Genbank | 181904 |
| 11105. | S63912 | Genbank | 399757 |
| 11106. | S73591 | Genbank | 688296 |
| 11107. | S74678 | Genbank | 241477 |
| 11108. | S75755 | Genbank | 861469 |
| 11109. | S77127 | Genbank | 998582 |
| 11110. | S79895 | Genbank | 1195555 |
| 11111. | U07086 | Genbank | 515984 |
| 11112. | U07088 | Genbank | 515986 |
| 11113. | U09500 | Genbank | 510270 |
| 11114. | U13948 | Genbank | 538276 |
| 11115. | U16660 | Genbank | 564064 |
| 11116. | U17032 | Genbank | 687592 |
| 11117. | U19822 | Genbank | 849082 |
| 11118. | U25165 | Genbank | 887792 |
| 11119. | U26424 | Genbank | 1203795 |
| 11120. | U30826 | Genbank | 1049079 |
| 11121. | U31906 | Genbank | 1173564 |
| 11122. | U32989 | Genbank | 993045 |
| 11123. | U33760 | Genbank | 995823 |
| 11124. | U36341 | Genbank | 1020318 |
| 11125. | U40369 | Genbank | 1103903 |
| 11126. | U41060 | Genbank | 1256000 |
| 11127. | U41384 | Genbank | 1145886 |
| 11128. | U41514 | Genbank | 1136284 |
| 11129. | U43604 | Genbank | 1171236 |
| 11130. | U47924 | Genbank | 1633547 |
| 11131. | U59305 | Genbank | 1695872 |
| 11132. | U65676 | Genbank | 1654350 |
| 11133. | U66048 | Genbank | 1519273 |
| 11134. | U67171 | Genbank | 2326174 |
| 11135. | U68140 | Genbank | 2406564 |
| 11136. | U69141 | Genbank | 1549326 |
| 11137. | U69274 | Genbank | 4097821 |
| 11138. | U69645 | Genbank | 1575614 |
| 11139. | U69668 | Genbank | 1850341 |
| 11140. | U75272 | Genbank | 1658285 |
| 11141. | U78303 | Genbank | 6648546 |
| 11142. | U79415 | Genbank | 1947070 |
| 11143. | U80017 | Genbank | 1737211 |
| 11144. | U82226 | Genbank | 1848270 |
| 11145. | U91321 | Genbank | 2951946 |
| 11146. | U91322 | Genbank | 2344876 |
| 11147. | U91328 | Genbank | 2088550 |
| 11148. | U91985 | Genbank | 2065560 |
| 11149. | U95742 | Genbank | 2339843 |
| 11150. | X00470 | Genbank | 36334 |
| 11151. | X03205 | Genbank | 36162 |
| 11152. | X06234 | Genbank | 34772 |
| 11153. | X12447 | Genbank | 28613 |
| 11154. | X15132 | Genbank | 34794 |
| 11155. | X15187 | Genbank | 37260 |
| 11156. | X15729 | Genbank | 38317 |
| 11157. | X17206 | Genbank | 34391 |
| 11158. | X52882 | Genbank | 311380 |
| 11159. | X55733 | Genbank | 288099 |
| 11160. | X56160 | Genbank | 37226 |
| 11161. | X62996 | Genbank | 13000 |
| 11162. | X65965 | Genbank | 36540 |
| 11163. | X68968 | Genbank | 452315 |
| 11164. | X70476 | Genbank | 298096 |
| 11165. | X71810 | Genbank | 297905 |
| 11166. | X74262 | Genbank | 397375 |
| 11167. | X74961 | Genbank | 439657 |
| 11168. | X74963 | Genbank | 439661 |
| 11169. | X74967 | Genbank | 439664 |
| 11170. | X74968 | Genbank | 439659 |
| 11171. | X78262 | Genbank | 587440 |
| 11172. | X78520 | Genbank | 854101 |
| 11173. | X81696 | Genbank | 940516 |
| 11174. | X83006 | Genbank | 929656 |
| 11175. | X83299 | Genbank | 603027 |
| 11176. | X85133 | Genbank | 728590 |
| 11177. | X89763 | Genbank | 976086 |
| 11178. | X95073 | Genbank | 2879814 |
| 11179. | X97675 | Genbank | 1834512 |
| 11180. | Y00371 | Genbank | 32466 |
| 11181. | Y00481 | Genbank | 29571 |
| 11182. | Y16241 | Genbank | 3378195 |
| 11183. | Z47087 | Genbank | 860989 |
| 11184. | Z48633 | Genbank | 1165102 |
| 11185. | Z55101 | Genbank | 1021142 |
| 11186. | Z55198 | Genbank | 1021239 |
| 11187. | Z57944 | Genbank | 1029175 |
| 11188. | Z70715 | Genbank | 1332506 |
| 11189. | Z73963 | Genbank | 6562178 |
| 11190. | Z82203 | Genbank | 3164070 |
| 11191. | Z82243 | Genbank | 3219581 |
| 11192. | Z83843 | Genbank | 2578095 |
| 11193. | Z84485 | Genbank | 3980102 |
| 11194. | Z85986 | Genbank | 4034056 |
| 11195. | Z93784 | Genbank | 2315175 |
| 11196. | Z94054 | Genbank | 2281935 |
| 11197. | Z95400 | Genbank | 4007180 |
| 11198. | AA001792 | EST | 1445606 |
| 11199. | AA004528 | EST | 1448105 |
| 11200. | AA010305 | EST | 1471331 |
| 11201. | AA011168 | EST | 1472195 |
| 11202. | AA018703 | EST | 1481977 |
| 11203. | AA026757 | EST | 1492555 |
| 11204. | AA033869 | EST | 1505752 |
| 11205. | AA035648 | EST | 1507458 |
| 11206. | AA037151 | EST | 1512259 |
| 11207. | AA037466 | EST | 1512565 |
| 11208. | AA044115 | EST | 1522029 |
| 11209. | AA044691 | EST | 1522912 |
| 11210. | AA064886 | EST | 1559007 |
| 11211. | AA069688 | EST | 1577108 |
| 11212. | AA075762 | EST | 1615693 |
| 11213. | AA079524 | EST | 1618416 |
| 11214. | AA082754 | EST | 1624812 |
| 11215. | AA082839 | EST | 1624914 |
| 11216. | AA083069 | EST | 1625127 |
| 11217. | AA086000 | EST | 1629567 |
| 11218. | AA090257 | EST | 1636749 |
| 11219. | AA090462 | EST | 1635038 |
| 11220. | AA094831 | EST | 1640416 |
| 11221. | AA095070 | EST | 1640663 |
| 11222. | AA102815 | EST | 1648660 |
| 11223. | AA114179 | EST | 1668065 |
| 11224. | AA115162 | EST | 1670359 |
| 11225. | AA131250 | EST | 1692812 |
| 11226. | AA135632 | EST | 1697068 |
| 11227. | AA136601 | EST | 1697846 |
| 11228. | AA137228 | EST | 1696979 |
| 11229. | AA143798 | EST | 1713203 |
| 11230. | AA147027 | EST | 1716398 |
| 11231. | AA147425 | EST | 1716796 |
| 11232. | AA149073 | EST | 1719363 |
| 11233. | AA152386 | EST | 1718701 |
| 11234. | AA159176 | EST | 1733969 |
| 11235. | AA160677 | EST | 1736062 |
| 11236. | AA161288 | EST | 1735524 |
| 11237. | AA164729 | EST | 1740889 |
| 11238. | AA167035 | EST | 1745410 |
| 11239. | AA169564 | EST | 1747952 |
| 11240. | AA186560 | EST | 1774659 |
| 11241. | AA187337 | EST | 1773530 |
| 11242. | AA193339 | EST | 1782929 |
| 11243. | AA193627 | EST | 1783038 |
| 11244. | AA195458 | EST | 1785222 |
| 11245. | AA203416 | EST | 1799143 |
| 11246. | AA203646 | EST | 1799365 |
| 11247. | AA209531 | EST | 1807492 |
| 11248. | AA224835 | EST | 1846123 |
| 11249. | AA226458 | EST | 1847804 |
| 11250. | AA228939 | EST | 1851758 |
| 11251. | AA228959 | EST | 1875053 |
| 11252. | AA234149 | EST | 1858244 |
| 11253. | AA234844 | EST | 1859336 |
| 11254. | AA243867 | EST | 1874696 |
| 11255. | AA248197 | EST | 1878786 |
| 11256. | AA248298 | EST | 1879135 |
| 11257. | AA251512 | EST | 1886494 |
| 11258. | AA251939 | EST | 1886900 |
| 11259. | AA258003 | EST | 1894435 |
| 11260. | AA279945 | EST | 1921410 |
| 11261. | AA280216 | EST | 1921817 |
| 11262. | AA280928 | EST | 1923815 |
| 11263. | AA282819 | EST | 1925735 |
| 11264. | AA283016 | EST | 1925940 |
| 11265. | AA285126 | EST | 1928107 |
| 11266. | AA287203 | EST | 1934228 |
| 11267. | AA287231 | EST | 1934257 |
| 11268. | AA287548 | EST | 1933432 |
| 11269. | AA287551 | EST | 1933435 |
| 11270. | AA291248 | EST | 1939226 |
| 11271. | AA297631 | EST | 1949965 |
| 11272. | AA304743 | EST | 1957144 |
| 11273. | AA311227 | EST | 1963627 |
| 11274. | AA311311 | EST | 1963732 |
| 11275. | AA313922 | EST | 1966472 |
| 11276. | AA315943 | EST | 1968272 |
| 11277. | AA318114 | EST | 1970440 |
| 11278. | AA321241 | EST | 1973568 |
| 11279. | AA323448 | EST | 1975773 |
| 11280. | AA324756 | EST | 1977052 |
| 11281. | AA329272 | EST | 1981516 |
| 11282. | AA329295 | EST | 1981698 |
| 11283. | AA333781 | EST | 1986035 |
| 11284. | AA342969 | EST | 1995205 |
| 11285. | AA351232 | EST | 2003571 |
| 11286. | AA354965 | EST | 2007283 |
| 11287. | AA367531 | EST | 2019848 |
| 11288. | AA375231 | EST | 2027550 |
| 11289. | AA378567 | EST | 2030927 |
| 11290. | AA378613 | EST | 2030963 |
| 11291. | AA380469 | EST | 2032787 |
| 11292. | AA385937 | EST | 2038325 |
| 11293. | AA393236 | EST | 2046205 |
| 11294. | AA393806 | EST | 2046773 |
| 11295. | AA397417 | EST | 2050430 |
| 11296. | AA401802 | EST | 2057286 |
| 11297. | AA401862 | EST | 2055881 |
| 11298. | AA410455 | EST | 2069561 |
| 11299. | AA410849 | EST | 2069957 |
| 11300. | AA411565 | EST | 2069149 |
| 11301. | AA417174 | EST | 2077255 |
| 11302. | AA420758 | EST | 2094637 |
| 11303. | AA424097 | EST | 2103058 |
| 11304. | AA425617 | EST | 2106373 |
| 11305. | AA429796 | EST | 2113003 |
| 11306. | AA436588 | EST | 2141502 |
| 11307. | AA441799 | EST | 2153683 |
| 11308. | AA442415 | EST | 2154293 |
| 11309. | AA452200 | EST | 2165869 |
| 11310. | AA458692 | EST | 2183599 |
| 11311. | AA468515 | EST | 2195049 |
| 11312. | AA469156 | EST | 2195690 |
| 11313. | AA469162 | EST | 2195696 |
| 11314. | AA478077 | EST | 2206711 |
| 11315. | AA478889 | EST | 2207523 |
| 11316. | AA480070 | EST | 2208221 |
| 11317. | AA481621 | EST | 2211173 |
| 11318. | AA484929 | EST | 2213996 |
| 11319. | AA485303 | EST | 2214522 |
| 11320. | AA485460 | EST | 2214679 |
| 11321. | AA487750 | EST | 2215181 |
| 11322. | AA492143 | EST | 2221705 |
| 11323. | AA493901 | EST | 2223742 |
| 11324. | AA503408 | EST | 2238375 |
| 11325. | AA508397 | EST | 2245900 |
| 11326. | AA513880 | EST | 2252301 |
| 11327. | AA514269 | EST | 2253937 |
| 11328. | AA522850 | EST | 2263562 |
| 11329. | AA524966 | EST | 2265894 |
| 11330. | AA548600 | EST | 2318882 |
| 11331. | AA553485 | EST | 2324024 |
| 11332. | AA555316 | EST | 2325855 |
| 11333. | AA558280 | EST | 2328757 |
| 11334. | AA565860 | EST | 2337499 |
| 11335. | AA568747 | EST | 2341801 |
| 11336. | AA570519 | EST | 2344499 |
| 11337. | AA572805 | EST | 2347333 |
| 11338. | AA578904 | EST | 2357088 |
| 11339. | AA579173 | EST | 2357357 |
| 11340. | AA601511 | EST | 2435136 |
| 11341. | AA609112 | EST | 2457540 |
| 11342. | AA625219 | EST | 2537604 |
| 11343. | AA629991 | EST | 2552602 |
| 11344. | AA631460 | EST | 2554071 |
| 11345. | AA635400 | EST | 2559242 |
| 11346. | AA635423 | EST | 2559265 |
| 11347. | AA640474 | EST | 2565724 |
| 11348. | AA641092 | EST | 2566342 |
| 11349. | AA643720 | EST | 2568938 |
| 11350. | AA648040 | EST | 2574469 |
| 11351. | AA651721 | EST | 2583373 |
| 11352. | AA651866 | EST | 2583518 |
| 11353. | AA651934 | EST | 2583586 |
| 11354. | AA653775 | EST | 2589929 |
| 11355. | AA657463 | EST | 2593617 |
| 11356. | AA658409 | EST | 2594563 |
| 11357. | AA659388 | EST | 2595542 |
| 11358. | AA662920 | EST | 2616911 |
| 11359. | AA676215 | EST | 2656737 |
| 11360. | AA687283 | EST | 2675474 |
| 11361. | AA693664 | EST | 2694602 |
| 11362. | AA700461 | EST | 2703424 |
| 11363. | AA702852 | EST | 2705965 |
| 11364. | AA702893 | EST | 2706006 |
| 11365. | AA722626 | EST | 2740333 |
| 11366. | AA723076 | EST | 2740783 |
| 11367. | AA740645 | EST | 2779237 |
| 11368. | AA740866 | EST | 2779458 |
| 11369. | AA742452 | EST | 2782034 |
| 11370. | AA768311 | EST | 2819326 |
| 11371. | AA769310 | EST | 2820548 |
| 11372. | AA788780 | EST | 2848900 |
| 11373. | AA826158 | EST | 2899470 |
| 11374. | AA827714 | EST | 2901273 |
| 11375. | AA832333 | EST | 2905432 |
| 11376. | AA837026 | EST | 2912225 |
| 11377. | AA843086 | EST | 2929604 |
| 11378. | AA848158 | EST | 2934676 |
| 11379. | AA856776 | EST | 2945078 |
| 11380. | AA889466 | EST | 3016345 |
| 11381. | AA903707 | EST | 3038830 |
| 11382. | AA905490 | EST | 3040613 |
| 11383. | AA908774 | EST | 3048179 |
| 11384. | AA923172 | EST | 3070481 |
| 11385. | AA926637 | EST | 3075534 |
| 11386. | AA927862 | EST | 3076606 |
| 11387. | AA939040 | EST | 3098953 |
| 11388. | AA985311 | EST | 3163836 |
| 11389. | AF001541 | EST | 2529713 |
| 11390. | AF001542 | EST | 2529714 |
| 11391. | AI000633 | EST | 3191187 |
| 11392. | AI018686 | EST | 3232484 |
| 11393. | AI024446 | EST | 3240059 |
| 11394. | AI027754 | EST | 3245193 |
| 11395. | AI038062 | EST | 3277256 |
| 11396. | AI041564 | EST | 3280758 |
| 11397. | AI056276 | EST | 3330142 |
| 11398. | AI065061 | EST | 6359333 |
| 11399. | AI081636 | EST | 3418428 |
| 11400. | AI088590 | EST | 3427649 |
| 11401. | AI095119 | EST | 3434095 |
| 11402. | AI114651 | EST | 6359996 |
| 11403. | AI124870 | EST | 3593384 |
| 11404. | AI133562 | EST | 6360878 |
| 11405. | AI142088 | EST | 3649545 |
| 11406. | AI174840 | EST | 6361237 |
| 11407. | AI184903 | EST | 3735541 |
| 11408. | AI216975 | EST | 3789629 |
| 11409. | AI217003 | EST | 3789657 |
| 11410. | AI217155 | EST | 3796970 |
| 11411. | AI247293 | EST | 3842690 |
| 11412. | AI253330 | EST | 3850451 |
| 11413. | AI253436 | EST | 3850391 |
| 11414. | AI262138 | EST | 3870341 |
| 11415. | AI267182 | EST | 3886349 |
| 11416. | AI267405 | EST | 3886572 |
| 11417. | AI267664 | EST | 3886831 |
| 11418. | AI267940 | EST | 3887107 |
| 11419. | AI270183 | EST | 3889350 |
| 11420. | AI275943 | EST | 3898217 |
| 11421. | AI276504 | EST | 3898778 |
| 11422. | AI278978 | EST | 3917212 |
| 11423. | AI278998 | EST | 3917232 |
| 11424. | AI281779 | EST | 3920012 |
| 11425. | AI289817 | EST | 3932841 |
| 11426. | AI299534 | EST | 3959119 |
| 11427. | AI318089 | EST | 4033869 |
| 11428. | AI345464 | EST | 4082670 |
| 11429. | AI357115 | EST | 4108736 |
| 11430. | AI361854 | EST | 4113475 |
| 11431. | AI366465 | EST | 4126154 |
| 11432. | AI366549 | EST | 4126238 |
| 11433. | AI370812 | EST | 4149565 |
| 11434. | AI381882 | EST | 4194663 |
| 11435. | AI401216 | EST | 4244303 |
| 11436. | AI422328 | EST | 4268259 |
| 11437. | AI425029 | EST | 4270960 |
| 11438. | AI432218 | EST | 4308644 |
| 11439. | AI434281 | EST | 4295081 |
| 11440. | AI439006 | EST | 4301300 |
| 11441. | AI439787 | EST | 4306763 |
| 11442. | AI457369 | EST | 4310238 |
| 11443. | AI457814 | EST | 4310683 |
| 11444. | AI458477 | EST | 4311056 |
| 11445. | AI460057 | EST | 4312938 |
| 11446. | AI473554 | EST | 4326599 |
| 11447. | AI474360 | EST | 4327405 |
| 11448. | AI476098 | EST | 4329143 |
| 11449. | AI500146 | EST | 4392128 |
| 11450. | AI521964 | EST | 4436099 |
| 11451. | AI538850 | EST | 4452985 |
| 11452. | AI564166 | EST | 4522623 |
| 11453. | AI571000 | EST | 4534374 |
| 11454. | AI590351 | EST | 4599399 |
| 11455. | AI627652 | EST | 4664452 |
| 11456. | AI636943 | EST | 4688273 |
| 11457. | AI656529 | EST | 4740508 |
| 11458. | AI675328 | EST | 4875808 |
| 11459. | AI679035 | EST | 4889217 |
| 11460. | AI684682 | EST | 4895976 |
| 11461. | AI685429 | EST | 4896723 |
| 11462. | AI687127 | EST | 4898421 |
| 11463. | AI691149 | EST | 4902451 |
| 11464. | AI692314 | EST | 4969654 |
| 11465. | AI695005 | EST | 4982905 |
| 11466. | AI696956 | EST | 4984856 |
| 11467. | AI741391 | EST | 5109679 |
| 11468. | AI768825 | EST | 5235334 |
| 11469. | AI791918 | EST | 5339634 |
| 11470. | AI797578 | EST | 5363050 |
| 11471. | AI798114 | EST | 5363576 |
| 11472. | AI798810 | EST | 5364282 |
| 11473. | AI805082 | EST | 5391648 |
| 11474. | AI808359 | EST | 5394925 |
| 11475. | AI821879 | EST | 5440958 |
| 11476. | AI824947 | EST | 5445618 |
| 11477. | AI863409 | EST | 5527516 |
| 11478. | AI866934 | EST | 5531041 |
| 11479. | AI879040 | EST | 5553089 |
| 11480. | AI885416 | EST | 5590580 |
| 11481. | AI902808 | EST | 6493195 |
| 11482. | AI904896 | EST | 6495283 |
| 11483. | AI907439 | EST | 6497869 |
| 11484. | AI907610 | EST | 6498045 |
| 11485. | AI908390 | EST | 6499070 |
| 11486. | AI909257 | EST | 6499937 |
| 11487. | AI916533 | EST | 5636388 |
| 11488. | AI935353 | EST | 5674223 |
| 11489. | AI936166 | EST | 5675036 |
| 11490. | AI936277 | EST | 5675147 |
| 11491. | AI950381 | EST | 5742691 |
| 11492. | AI951118 | EsT | 5743428 |
| 11493. | AI972980 | EST | 5769806 |
| 11494. | AI983045 | EST | 5810264 |
| 11495. | AJ223808 | EST | 2894149 |
| 11496. | AL036115 | EST | 5927646 |
| 11497. | AL037799 | EST | 5928278 |
| 11498. | AL037810 | EST | 5407145 |
| 11499. | AL040146 | EST | 5409111 |
| 11500. | AL044891 | EST | 5433089 |
| 11501. | AL118933 | EST | 5924832 |
| 11502. | AL134261 | EST | 6602448 |
| 11503. | AW002364 | EST | 5849280 |
| 11504. | AW006621 | EST | 5855399 |
| 11505. | AW008737 | EST | 5857515 |
| 11506. | AW024055 | EST | 5877585 |
| 11507. | AW052046 | EST | 5914405 |
| 11508. | AW068542 | EST | 6023540 |
| 11509. | AW090498 | EST | 6047842 |
| 11510. | AW090549 | EST | 6047893 |
| 11511. | AW129659 | EST | 6117603 |
| 11512. | AW130697 | EST | 6132229 |
| 11513. | AW131596 | EST | 6133203 |
| 11514. | AW137451 | EST | 6141769 |
| 11515. | AW148750 | EST | 6196646 |
| 11516. | AW160361 | EST | 6299394 |
| 11517. | AW167692 | EST | 6399217 |
| 11518. | AW170764 | EST | 6402289 |
| 11519. | AW175604 | EST | 6441745 |
| 11520. | AW175646 | EST | 6441787 |
| 11521. | AW175662 | EST | 6441803 |
| 11522. | AW175786 | EST | 6441823 |
| 11523. | AW293039 | EST | 6699675 |
| 11524. | AW299971 | EST | 6709648 |
| 11525. | AW302028 | EST | 6711705 |
| 11526. | AW363699 | EST | 6868349 |
| 11527. | AW368592 | EST | 6873242 |
| 11528. | AW369681 | EST | 6874335 |
| 11529. | AW372279 | EST | 6877037 |
| 11530. | AW373663 | EST | 6878317 |
| 11531. | AW373685 | EST | 6878339 |
| 11532. | AW380068 | EST | 6884727 |
| 11533. | AW380640 | EST | 6885299 |
| 11534. | AW385934 | EST | 6890593 |
| 11535. | AW387746 | EST | 6892405 |
| 11536. | AW390087 | EST | 6894746 |
| 11537. | AW411336 | EST | 6936877 |
| 11538. | AW419384 | EST | 6947316 |
| 11539. | AW439494 | EST | 6974800 |
| 11540. | AW450987 | EST | 6991753 |
| 11541. | AW514896 | EST | 7152978 |
| 11542. | AW572781 | EST | 7237514 |
| 11543. | AW577352 | EST | 7252401 |
| 11544. | AW577426 | EST | 7252475 |
| 11545. | AW580534 | EST | 7255583 |
| 11546. | AW605197 | EST | 7309938 |
| 11547. | AW607394 | EST | 7312135 |
| 11548. | AW794726 | EST | 7846596 |
| 11549. | AW811239 | EST | 7904233 |
| 11550. | AW851829 | EST | 7947346 |
| 11551. | AW864017 | EST | 7998067 |
| 11552. | AW879411 | EST | 8041421 |
| 11553. | AW889716 | EST | 8053921 |
| 11554. | AW890845 | EST | 8055050 |
| 11555. | AW935467 | EST | 8110873 |
| 11556. | AW939901 | EST | 8115347 |
| 11557. | AW954494 | EST | 8144177 |
| 11558. | AW970055 | EST | 8159900 |
| 11559. | BE046480 | EST | 8363533 |
| 11560. | BE086429 | EST | 8476822 |
| 11561. | BE144858 | EST | 8607582 |
| 11562. | BE168126 | EST | 8630847 |
| 11563. | BE179948 | EST | 8659009 |
| 11564. | D57966 | EST | 964588 |
| 11565. | H12642 | EST | 877462 |
| 11566. | H26466 | EST | 895589 |
| 11567. | H60498 | EST | 1013330 |
| 11568. | H64038 | EST | 1018839 |
| 11569. | H66929 | EST | 1025669 |
| 11570. | H72624 | EST | 1044440 |
| 11571. | H84066 | EST | 1062737 |
| 11572. | H85791 | EST | 1067370 |
| 11573. | N24491 | EST | 1138641 |
| 11574. | N30415 | EST | 1148935 |
| 11575. | N41317 | EST | 1165348 |
| 11576. | N43818 | EST | 1182346 |
| 11577. | N49466 | EST | 1190632 |
| 11578. | N51481 | EST | 1192647 |
| 11579. | N56475 | EST | 1199323 |
| 11580. | N64522 | EST | 1212351 |
| 11581. | R00988 | EST | 750724 |
| 11582. | R10015 | EST | 761971 |
| 11583. | R17957 | EST | 771567 |
| 11584. | R59333 | EST | 830028 |
| 11585. | R80340 | EST | 856621 |
| 11586. | R94022 | EST | 969417 |
| 11587. | R97758 | EST | 983418 |
| 11588. | T60687 | EST | 663724 |
| 11589. | T86669 | EST | 715021 |
| 11590. | T88675 | EST | 717027 |
| 11591. | U46401 | EST | 1245069 |
| 11592. | W73836 | EST | 1383989 |
| 11593. | W80468 | EST | 1391485 |
| 11594. | Z19286 | EST | 29115 |
| 11595. | Z32861 | EST | 483725 |
| 11596. | Z42255 | EST | 565359 |
| 11597. | A02479 | NUCPATENT | 512039 |
| 11598. | A06583 | NUCPATENT | N/A |
| 11599. | A06593 | NUCPATENT | N/A |
| 11600. | A06599 | NUCPATENT | N/A |
| 11601. | T58248 | NUCPATENT | 660109 |
| 11602. | V60015 | NUCPATENT | N/A |
| 11603. | V69855 | NUCPATENT | N/A |
| 11604. | X04366 | NUCPATENT | 29663 |
| 11605. | X40569 | NUCPATENT | N/A |
| 11606. | X40607 | NUCPATENT | N/A |
| 11607. | Z15132 | NUCPATENT | 65134 |
| 11608. | Z18355 | NUCPATENT | 30767 |
| 11609. | Z24502 | NUCPATENT | 394851 |
| 11610. | Z40833 | NUCPATENT | 566574 |
| 11611. | Z41384 | NUCPATENT | 567528 |
| 11612. | Z80029 | NUCPATENT | 3153090 |
| 11613. | Z80662 | NUCPATENT | 1770975 |
| 11614. | Z94215 | NUCPATENT | 1945209 |
| 11615. | Z97314 | NUCPATENT | 2245294 |
| 11616. | AC37143 | PREPATNUC | N/A |
| 11617. | AC40326 | PREPATNUC | N/A |

TABLE 2

| | |
|---|---|
| Page 1/3 | |
| Prepatent Sequences from cMhqao library | |
| Sequence 8515: Found in Patent publication WO99/46374 | |
| CCCCCCTCGNGAGTACTTCTAGAA11AATTAACGCGGGGTCCATTCTGCGCAGTATCTCA | SEQ ID NO:1 |
| ACTGCCGTTCAACAATCGCAAGAGGAAGGTGGAGCAGGTTTCTTCATCTTACAGUGAGA | |
| AAACAGAGACTCAGAAGGGCTTCTTAGTTCATGTTTCCCTTAGCGCCTCAGTGATTTTTT | |
| CATGGTGGCTTAGGCCAAAAGAAATATCTAACCATTCAATTTATAAATAAUAGGTCCCC | |
| AACGAATTAAATATTATGTCCTACCAACTTATTAGCTGCTTGAAAAATATAATACACATA | |
| AATAAAAAAATATATTTTTCATTTCTATTTCATTGTTAATCACAACTACUACTAAGGAG | |
| ATGTATGCACCTATTGGACACTGTGCAACTTCTCACCTGGAATGAGAUGGACACTGCTG | |
| CCCTCATTTTCTGCTCCATGTTGGTGTCCATATAGTACTTGATTTTTTATCAGATGGCCT | |
| GGAAAACCCAGTCTCACAAAAATATGAAATTATCAGAAGGAUATAGTGCAATCTTATGT | |
| TGAAAGAATGAACTACCTCACTAGTAGTTCACGTGATGTCTGACAGATGTTGAGTTTCATT | |
| Sequence 8516: Found in Patent publication WO00/18916.1065 | |
| GCCCCCCCTCGGAAGTCTTCTAGNATTAATTAACGCGGGGGGAAAGCCTTCAGTCCTTTC | SEQ ID NO:2 |
| TGTTTCTTTCAACTACGTGAAAGGATTCACAGTGGAGAAAGACCCTGTAAGATAATTGGC | |
| TTTAAATTACGAGAGACTTGTGATAGGACAGTAAAACCTAGAGTTGGAGTTGGATCTCTG | |
| GATTGTGTTATGTCAGTGTTGGTAGGTAGGTTCTAGATTTCCCAGAATCCATTCCATTT | |
| GTGATTCCATGATACAATTCACCAGTAACCTATCTTACATGAGATTCGGAAGTAAGTTAA | |
| GAAGGCATTAGTCATGGTTTGGAAGCACCATACAGGGAGACAGCTGTGTGAATACAGGCT | |
| GTATGGACACTTGCTTCCATCCCATTTTCCTGCTTCTTTGGGTTGCCAATCAAGAGTATC | |
| CTCAAAACGACTTGACTTTAATTTTCTCGGAGGTGATAGGCTTCCACACAGGTCTCCAGA | |
| AGCCCTGCATTGAATATCGATCCACACTTTGGTTTTCCTTCAGACATTATTATGTCTGTA | |
| CTAGGCAACTAATTCAGACTGTCCTGGTTGGGAATATTCTGTGATGCTCTGACTCCCCTAGT | |
| Sequence 8517: Found in Patent publication WO00/44900.90 | |
| CGGGCCCCCCCTCGGAAGTCTTCTAGAATTAATTAACGCGGGGGGAGCGGGCGAGCGGAG | SEQ ID NO:3 |
| TTAGCAGGGCTTTACTGCAGAGCGCGCCGGGCACTCCAGCGACCGTGGGGATCAGCGTAG | |
| GTGAGCTGTGGCCTTTTGCGAGGTGCTGCAGCCATAGCTACGTGCGUCGCTACGAGGAT | |
| TGAGCGTCTCCACCCAGTAAGTGGGCAAGAGGCGGCAGGAAGTGGGTACGCAGGGGCGCA | |
| AGGCGCACAGCCTCTAGACGACTCGCTTTCCCTCCGGCCAACCTCTGAAGCCGCGTCCTA | |
| CTTTGACAGCTGCAGGGCCGCGGCCTGGTCTTCTGTGCTTCACCATCTACATAATGAATC | |
| CCAGTATGAAGCAGAAACAAGAAGAAATCAAAGAGAATATAAAGAATAGTTCTGTCCCAA | |
| GAAGAACTCTGAAGATGATTCAGCCTTCTGCATCTGGATCTCTTGTTGGAAGAGAAAATG | |
| AGCTGTCCGCAGGCUGTCCAAAAGGAAACATCGGAATGACCACTTAACATCTACAACTT | |
| CCAGCCCTGGGGTTATTGTCCCAGAATCTAGTGAAAATAAAAATCTTGGAGGAGTCACC | |
| Sequence 8518: Found in Patent publication WO00/49043.21 | |
| GTACCGGGCCCCCCCCTCGGAAATACTTCTAGNATTAATTAACGCGGGTGAAGAAAAAAA | SEQ ID NO:4 |
| ACGACTTGAGGAAAAACAAAGAGCAGCCCGCAAAAACAGGTCCAAGTCAGAAGAGGACTG | |
| GAAGACGAGGTGGTTCCATCAAGGTCCTAATCCCTACAATGGAGCACAGGACTGGATTTA | |
| CTCTGGCAGCTACTGGGACAGAAATTACTTCAATTTGCCTGACATTTATTAAAATGCATA | |
| CAAGTCAGGGTGTTTGGCTAATCTACAAATAAGTCTTAAACCTATGTTTTTAAATTTTTT | |
| TCCCTTGGTTTCTACTTATCTTTTAAAAAAAAAATGAAAAAACACTCATGAGATAACTGC | |
| ATTTCACCCAACAAAAGCAGGGTATAAGGCGATATTGGTGATGAAAGTCTTAGGAAAAAT | |
| GCATAATTTTGCTATAAAATGTACTTATTTGGAATACTATTTTATATAGAGGTAAGAGAA | |
| CACTGCTGGGGAATATGCTTTTTATGGTTGCTGTTGCCATATTTACTGAAGGTTTATACC | |
| TAAATGTAACTTTAGCTTTATGGAACTATATAGTAATCCCAAATCAAGTTATTTTGAATA | |
| TTTTTATGCTGTCATGCT | |
| Prepatent Sequences from cMhqaq library | |
| Sequence 3683: Found in Patent publication U56096516.21 | |
| ATGCTGCACCAACTGTATCCATCTTCCCAGCATCCAGTGAGCAGTTAACATCTGGAGGTG | SEQ ID NO:5 |
| CCTCAGTCGTGTGCTTCTTGAACAACTTCTACCCCAAAGACATCAATGTCAAGTGGAAGA | |
| TTGATGGCAGTGAACGACAAAATGGCGTCCTGAACAGTTGGACTGATCAGGACAGCAAAG | |
| ACAGCACCTACAGCATGAGCAGCACCCTCACGTTGACCAAGGACAAGTTTGAACGACATA | |
| ACAGC | |
| Page 2/3 | |
| Prepatent Sequences from cMhqar library | |
| Sequence 5658: Found in Patent publication WO00/12544 | |
| CCGGGCCCCCCCTCGGAAGTCTTCTAGAATTAATTAACGCGGGTATATTGTGGCATATAG | SEQ ID NO:6 |
| TCAACATGGAAGTAGACCAGCTCTGCTGATTTGAAATTTAGATTTTTTAAATTATGTACT | |
| GGGGACAGGTTTTTGTCGCTTTACATTGCTTCCTAGTTTACAGCATGATGCAAATGATTT | |
| TCTAACTTAGTGTTAGGAGAAATTATTTTCCATCTTTAACCTCTTAGTTGTCTAAGAGTT | |
| AAATATTACTGAAUTCAGACGTTCAAATTGATCATCACAAATCCTTTAAAACAATTACC | |
| TAAAAGAAACCAAAAATCCTGCCTTCTTTGTGGGGGAGGGGGGAGAGAGGGGAAGGAAAT | |
| GGAACAAGTTGTGTTTGTGTTAGCATGTGGGTGATGTAAACTTCAAAUGGGAGATGTTC | |
| CGACCCCCACTCCCATATAAGCATTCAATCAGTGTAACTTGCAAAATGCATAAACATCCG | |
| ACAGTTTGAATTTATAGTGTTGATGGAAGAAATCATTTTTAATGTGTACTGTAAAACUG | |
| AAATACTCAGGAGCTGAGTGAATATGT | |
| Sequence 5659: Found in Patent publication WO00/37634.t10 | |
| GCCCCCCCTCGGAAGTCUCTAGNATTAATTAACGCGGGTTTACATACAGATGGCAAACT | SEQ ID NO:7 |
| TCATTTCCTTTTCTCTAATGCAACAAGGTCATCCCAAGATCAGGCTTCCTTCAGTTTC | |
| TGTGGTAAGTAGTGATGGACACTTATGGAGTTTTCAGAGACTTATGCATTGGGTAACAAG | |
| GCACTGCAAGAAACCCCAGATAGCACAGCATCATCTCACATTACACCACATCACATCAA | |
| CATCGATGCTAGGAGGTCTAAAGCTGATGCCACCTTCAGAGCTGCAAGTATCCAAAAGAC | |
| TCCACTCATGACACCAGCTCACTCCTGAGTCACATCAGCCCCTTTCAGAGTCACAGCCCC | |
| AGAACTAAGGCTATCCATATGGGTTGATTATAAAATCCAAAAAGCAGCTCCAAATATTAG | |
| AAACACTTATTTGTCTGGCAGACACATTCCCTAATTTAAAGCCTCTTATACTGTGCTTAA | |
| AAATATTCAACAGTCTTAGCAAAATGACAAAATAAACAGGTAGCAGCCATGGGGTGGGCA | |
| GGGTAAGGTGGCGTTGAAAGAAAAGGCATTTAGCAAGGTAAGTAGAGAGTGCTGATGG | |
| Sequence 5660: Found in Patent publication WO00/43420.8 | |
| CCGGGCCCCCCCTCGAAATACTTCTAGNATTAATTAACGCGGGGGGGGAAGCCATCTGCC | SEQ ID NO:8 |
| TTCGAGCCTGCCACTGAAATGCAAAAGTCTGTCCCAAATAAAGCCTTGGAATTGAAAAAT | |
| GAACAAACATTGAGAGCAGATGAGATACTCCCATCAGAATCCAAACAAAAGGACTATGAA | |
| GAAAATTCTTGGGATACTGAGAGTCTCTGTGAGACTGTTTCACAGAAGGATGTGTGTTTA | |
| CCCAAGGCTGCGCATCAAAAAGAAATAGATAAAATAAATGGAAAATTAGAAGGGTCTCCT | |
| GTTAAAGATGGTCTTCTGAAGGCTAACTGCGGAATGAAAGTTTCTATTCCAACTAAAGCC | |
| TTAGAATTGATGGACATGCAAACTTTCAAAGCAGAGCCTCCCGAGAAGCCATCTGCCTTC | |
| GAGCCTGCCATTGAAATGCAAAAGTCTGTTCCAAATAAAGCCTTGGAATTGAAGAATGAA | |
| CAAACATTGAGAGCAGATGAGATACTCCCATCAGAAATCAAACAAAGGACTATGAAGAAA | |
| GTTCTTGGGATTCTG | |
| Prepatent Sequences from cMhqaj library | |
| Sequence 10524: Found in Patent publication WO99/09168 | |
| ACAGTTCATAAGGCCTTTGTCATTGTTAGTCACCTTCGATGTGAATATCAGTAGGGCCAT | SEQ ID NO:9 |
| TAAATTAAAGCTGGCCTAAAAAGTTTTCAAAGTGCTACTGTTTCCAACTGATGAAATCTT | |
| CAGTTTTCTGGATTTTCTGGGTGACGATTATTTTCAATTCTTTTTTTCTGGTGATATATA | |
| CAAGAAGTTATAGCAGATATATAAAGGGAAGATCAGAAGCCTGCTGTCCAAGTTCATCAC | |
| CACTTGTTCCTGATCCTTTTCAAGTGTAAGTATTTGATAGCCAGTTGTTCTACTACATAT | |
| TAGTTTTCCACTACTATCAAAAGAAGCCAACGTAATCTAAATGCTATGCTCCTTCGAGG | |
| CTGTAAACTGACAGATCCGCTACATGGCTGATTCAGTGTATTGCGTTTGAAAATGATGTA | |
| TCGATTGGTGTAAGTTACAAGTAGGTCGAAGCCGAAGTTAAAACCTGTCCAACGCCAGCA | |
| GTATTCACCATCTTTGGCAAGCTTTCTACCACACCTCATGCTGTTTCCCTCTAGTTTCTT | |
| CTTTATTGATUCTTGAGGCCCCACCTCACTGTCCTGTTCTGCCCGGAGCATAGCAAACC | |
| ACTGCTGTTTATACACAGAAGANGAGCCATTCTTGAAAGGNACCTCCGGCCCGTCTAGAA | |
| CTAGGTGGAATCCCCCCG | |
| Sequence 10525: Found in Patent publication WO00/20590.13 | |
| CCNCCGCGGGGGCGGCCGAGGTNCCTTNCATTGGATTGTCCATGGNGCATCGCTTTAGCA | SEQ ID NO:10 |
| AGCGGCGTGACTCTCCACTGCCCGTCATCTTGGCCAATATCTATCTGCTGGTTCCTCCTG | |
| TGCTCAACCCAATTGTCTATGGAGTGAAGACAAAGGAGATTCGACAGCGCATCCTTCGAC | |
| TTTTCCATGTGGCCACACACGCTTCAGAACCCTAGGTGTCAGTGATCAAACTTCTTTTCC | |
| ATTCAGAGTCCTCTGATTCAGATTTNAATGTTAACATTGTGGAAGACAGTNTTCANAAAA | |
| Page 3/3 | |
| AAAATTTCCTTAATAAAAATACAACTCANATCCTTCAAATATGAAACTGGTTGGGGAATC | |
| TCCATTTTTTCAATANTATTTNCTTCTTNGNNNNCNTGCTACATATAA11ATTAATACCC | |
| TGACTAGGTTGNGGTTGGANGGTTATTACTTTTGATACCATGCAGTCCAAATCTAAACT | |
| GCTTCTACTGATGGTTTACAGCATTCTGAGATAAGAATGGTCATCTAGAGAACATTTGCCAA | |
| Sequence 10526: Found in Patent publication WO00/18916.893 | |
| ACTCCACCGCGGTGGCGGCCGNGGTACCATGGTACAAACCAGCATGCCGGGNAACACTTG | SEQ ID NO:11 |
| TCCTCTGTTTGCCTCAAATAAAGATTATTAGTGCTGGGCACAAGTATATGGAACCTCTGC | |
| AGGAGATTCCATTTGTTATCCCACGACCCATCCTTGAAGAAGGTGATGCTTTTCCTTGGA | |
| CGATCAGCTTGCATAATTTCAGCATATATACCCTTCTTGGAAAACAAGTGACACTTTGCC | |
| TAGTGGAACCTATGGGTTGCACCTCCACTCTAGCTGTCACGTCTCAAAAACTGCTTGCTA | |
| CGGGACCTGATACACGACATTCATTTGTTGTCTGTCTCCATGTTGACCTAGAGTCACTAG | |
| AGATAAAATGCTCTAATCCCCA | |
| Sequence 10527: Found in Patent publication WO00/29422.34 | |
| CCGCGGTGGCGGCCGAGGTACCTCCAGNACAGGNAGGATCTGCCAGTTACTACUGGAAC | SEQ ID NO:12 |
| CTCCTCCCUCTCTCTTCCCTGTATGATGTATTTTCTTTCATGGCTTAGTGGTAGCTCAA | |
| AGCTCAAGGTGACCAGAATGATTCCCTTACAGCAGTGGUCCCAAACATTGATGTGCAAC | |
| AGAACAACTTGGGTAGTUGTAGAAAGGGCAGATTTCCTGGCCCTATTCCCAGAGATTCG | |
| GAATTAGTAGGTTTGAGGTGGGGCCCAGGAATCTGCACTCAAATGTGCTACTCTCAGGAT | |
| CTGACCTAAGGGGCCTTTCATATGTGGCCCCCTTTCCTCACCGGAGGACATAAGTGCATC | |
| ACACAAACCTCTTGCTGTGGGCACTTCCTTTGGGGTTAAAAATGGGACCATTTGATTAAG | |
| GCAACCATTGCTAACCATCTTACTTCCCTGGGCTGGAGAAATATCAATGTCACATTTTA | |
| TGTTCTTAAATCTCAGGGCCACAGATTTCCTTTGACAATGACATGCTCTGTAACATGAA | |
| ATTTATATCCTAAAGCCTTTTGTCCTTCCTTCTT | |
| Sequence 10528: Found in Patent publication WO00/29583.35 | |
| ACAGGTCCCCGGAGGCATCCTGGCTGGGTGGGAAGTTTCTGGCGGTCACGCCCTGTCCGC | SEQ ID NO:13 |
| TTTCGCTCCAGGTCACACTGAGTGGCTCCTGGGGGAAGAAGCCCTGGACCAGGCAGGCGA | |
| TGACCACGTTCCCATCTGGCTGGGTGCTGCAGAGGCTCAGCGGGAAGACCTTGGGGCTGG | |
| TCGGGGATGCTGAGGAGACGGTGACCAGAGTTCCCTGGCCCCAGGGGTCAATCGACTGTT | |
| CGTCAAAATACTCGGCTTTTGCCGCACAGTGATATAGAGCCGTGTCTGCAGCGGTCACAG | |
| AGGTCAACTTCAGGGAGAACTGATTCTTGGACGTGTCAAAGGACATGGTGACTCGACTCT | |
| TGAGGGACGGGTTGTGGAAAATACTCCCATAATATGCGATGGTCCCAACCCACTCCAGGC | |
| CCTTCCTTGGGGGGCTTGGCNGNATCCACCCCCANAAGGAACCACTACTGGTAATGGANA | |
| CACCANANACANTGCAAGTGANGGACANGGTCTNCNAAGGCTTNACCANTCCTGGG | |
| Prepatent Sequences from cMhqal library | |
| Sequence 11616: Found in Patent publication WO99/64576 | |
| ACCTTTTTTTTTTTTTTTTTTTTTTTGGGGAAGGGGGGGTAAACCCAAAACCCTTTTTAA | SEQ ID NO:14 |
| ATGGGGGGGTTTTTTAACCCCCACATGGGGGGAAAAATTTTTACCCTTTTTAAAAAGGTT | |
| TTTTCCAAAGGCAAAAAAAACTTTCCTTTTTTGAACAAAATTAAATTTCAAAGGGGGTT | |
| TTAAGGGGTTTTGGGGCCAAATTAAAAGGGAAACAAAATTTTTTTTTGAAAAACCATTT | |
| TTCACCAGGGTGGGGGGGTTTGCCCC | |
| Sequence 11617: Found in Patent publication WO00/18916.629 | |
| ACGCGGGGTGGGAAATAAATCAGGGCGGCCTTTGGCATTTCACAGGCATAACTCCTCTAT | SEQ ID NO:15 |
| TCTTCTGCTGCAAATTTAGAGCTTACAAGATGCTAATGGCACCTCAGTTTCCAGGATAGT | |
| CTCTGCAGACTGCTTTCACTCAGCAGGCTTCCTTTCATGATGGCTGCCAGTAACAGCTAC | |
| CCCTCATTTACATCCAGCAGAGATGAGGGGTATCTCTTCGGGGAGTTCGTGCAAAAAAAA | |
| GCAAGAAAAGCTGAAATCTTCTCACTTCAAAGGAATGGCACATGACAGCACTTAGCCAATA | |

TABLE 3A
Liver Mets

| | | | | |
|---|---|---|---|---|
| GI\|2166979 | GI\|2955793 | GI\|2432430 | GI\|2617817 | GI\|2111406 |
| GI\|1192046 | GI\|2179508 | GI\|771572g | GI\|1379503 | GI\|3154368 |
| GI\|2159454 | GI\|2994907 | GI\|3149107 | GI\|3001978 | GI\|2183756 |
| GI\|1548168 | GI\|2177373 | GI\|816068g | GI\|2179345 | GI\|3405896 |
| GI\|2525281 | GI\|831676g | GI\|1102589 | GI\|1182737 | GI\|3231129 |
| GI\|1212669 | GI\|2111121 | GI\|2618146 | GI\|1023769 | GI\|2657287 |
| GI\|1792569 | GI\|2110926 | GI\|2159071 | GI\|890830g | GI\|2839089 |
| GI\|2714531 | GI\|1472203 | GI\|2051032 | GI\|1886979 | GI\|2240463 |
| GI\|4110658 | GI\|2945934 | GI\|1496296 | GI\|1486874 | GI\|2165555 |
| GI\|2713314 | GI\|1719469 | GI\|1196234 | GI\|1436888 | GI\|1471250 |
| GI\|2142138 | GI\|2217195 | GI\|3070014 | GI\|2155679 | GI\|3147260 |
| GI\|1009937 | GI\|891744g | GI\|1218594 | GI\|2053778 | GI\|2189568 |
| GI\|2216635 | GI\|2141183 | GI\|1692160 | GI\|2219395 | GI\|2107186 |
| GI\|767048g | GI\|885481g | GI\|1064170 | GI\|3240393 | GI\|2241054 |
| GI\|2835208 | GI\|2178011 | GI\|1687643 | GI\|835476g | GI\|1679074 |
| GI\|2631015 | GI\|891548g | GI\|2178341 | GI\|1925386 | GI\|2968058 |
| GI\|883043g | GI\|2115783 | GI\|2214433 | GI\|2541504 | GI\|3230029 |
| GI\|656953g | GI\|1231112 | GI\|2617430 | GI\|1687588 | GI\|869870g |
| GI\|673514g | GI\|2954944 | GI\|2215024 | GI\|2569563 | GI\|697470g |
| GI\|2167419 | GI\|2070642 | GI\|1751182 | GI\|3869944 | GI\|1198084 |
| GI\|865164g | GI\|2112377 | GI\|3181670 | GI\|821229g | GI\|4124377 |
| GI\|3231778 | GI\|789775g | GI\|967342g | GI\|2432144 | GI\|1634222 |
| GI\|1741729 | GI\|958710g | GI\|694526g | GI\|2177487 | GI\|3700837 |
| GI\|1147099 | GI\|1544421 | GI\|3182593 | GI\|2718216 | GI\|3031088 |
| GI\|1697431 | GI\|1224880 | GI\|1940891 | GI\|675043g | GI\|3154160 |
| GI\|3203959 | GI\|2166602 | GI\|2631056 | GI\|1182504 | GI\|3058536 |
| GI\|1211438 | GI\|1191963 | GI\|2432803 | GI\|1697746 | GI\|3057539 |
| GI\|2930110 | GI\|2834575 | GI\|2703567 | GI\|5054970 | GI\|1764615 |
| GI\|1188883 | GI\|839785g | GI\|2107859 | GI\|3239652 | GI\|759218g |
| GI\|2184225 | GI\|1398641 | GI\|1271153 | GI\|2524189 | GI\|3230283 |
| GI\|3162085 | GI\|1319591 | GI\|717200g | GI\|2179534 | GI\|2138791 |
| GI\|2177884 | GI\|2834870 | GI\|892219g | GI\|3190810 | GI\|985443g |
| GI\|1231416 | GI\|2185067 | GI\|3094650 | GI\|2834950 | GI\|3182061 |
| GI\|2955433 | GI\|1670627 | GI\|1520983 | GI\|2968580 | GI\|2107862 |
| GI\|2715060 | GI\|1426069 | GI\|1042590 | GI\|919369g | GI\|2537954 |
| GI\|2141192 | GI\|1733156 | GI\|1435274 | GI\|1919792 | GI\|1689729 |
| GI\|1549730 | GI\|3232370 | GI\|685981g | GI\|2216792 | GI\|1329704 |
| GI\|1124481 | GI\|2051509 | GI\|2957943 | GI\|1695573 | GI\|2835197 |
| GI\|2209964 | GI\|861690g | GI\|2839059 | GI\|2206902 | GI\|2106972 |
| GI\|1152351 | GI\|704523g | GI\|994995g | GI\|680496g | GI\|3413101 |
| GI\|1149200 | GI\|1773895 | GI\|2955292 | GI\|1312710 | GI\|1047231 |
| GI\|2155757 | GI\|3154742 | GI\|1319036 | GI\|1188649 | GI\|3215096 |
| GI\|2185559 | GI\|2100543 | GI\|2524970 | GI\|2969813 | GI\|1426013 |
| GI\|2077802 | GI\|1748319 | GI\|1785305 | GI\|4124202 | GI\|1859553 |
| GI\|3058260 | GI\|2703544 | GI\|1016909 | GI\|1157469 | GI\|3116842 |
| GI\|2191680 | GI\|2538277 | GI\|2557019 | GI\|3048520 | GI\|751040g |
| GI\|2718954 | GI\|1860336 | GI\|813202g | GI\|1694462 | GI\|1109117 |
| GI\|2229975 | GI\|1269934 | GI\|2218334 | GI\|2165947 | GI\|880662g |
| GI\|1502260 | GI\|2524934 | GI\|1389303 | GI\|1426093 | GI\|2630541 |
| GI\|1068723 | GI\|1698017 | GI\|2101007 | GI\|1118736 | GI\|2204485 |
| GI\|2112539 | GI\|1099415 | GI\|1685895 | GI\|3231631 | GI\|2955952 |
| GI\|2106462 | GI\|666684g | GI\|2185500 | GI\|1109119 | GI\|2220971 |
| GI\|1190314 | GI\|2432100 | GI\|3173567 | GI\|2103515 | GI\|900124g |
| GI\|891906g | | | | |
| GI\|2240425 | | | | |
| GI\|2163639 | | | | |
| GI\|2178961 | | | | |
| GI\|2229773 | | | | |
| GI\|1156914 | | | | |
| GI\|2155634 | | | | |
| GI\|1426474 | | | | |
| GI\|2214720 | | | | |
| GI\|994204g | | | | |
| GI\|874758g | | | | |
| GI\|3151403 | | | | |
| GI\|2215333 | | | | |
| GI\|1776165 | | | | |
| GI\|2183390 | | | | |
| GI\|1057622 | | | | |
| GI\|3190867 | | | | |
| GI\|1137533 | | | | |
| GI\|2556872 | | | | |
| GI\|2077689 | | | | |
| GI\|882949g | | | | |
| GI\|1328950 | | | | |
| GI\|4148131 | | | | |
| GI\|2214845 | | | | |
| GI\|3181720 | | | | |
| GI\|815062g | | | | |
| GI\|1271101 | | | | |
| GI\|1791370 | | | | |
| GI\|2080358 | | | | |
| GI\|1313788 | | | | |
| GI\|713327g | | | | |
| GI\|1026014 | | | | |
| GI\|3069635 | | | | |
| GI\|782116g | | | | |
| GI\|1625747 | | | | |
| GI\|1212234 | | | | |
| GI\|1753559 | | | | |
| GI\|3250604 | | | | |
| GI\|2945645 | | | | |
| GI\|2713337 | | | | |
| GI\|857060g | | | | |
| GI\|1615592 | | | | |
| GI\|1740942 | | | | |
| GI\|1153038 | | | | |
| GI\|2056563 | | | | |
| GI\|2165924 | | | | |
| GI\|771553g | | | | |

TABLE 3B
Bone Mets

| | | | | |
|---|---|---|---|---|
| GI\|2166979 | GI\|2112539 | GI\|1109117 | GI\|2557019 | GI\|994995 |
| GI\|1192046 | GI\|2077802 | GI\|1218594 | GI\|2101007 | GI\|2552195 |
| GI\|2159454 | GI\|1860336 | GI\|1633650 | GI\|659253 | GI\|3231631 |
| GI\|1792569 | GI\|3058260 | GI\|2219395 | GI\|2185067 | GI\|2215024 |
| GI\|2713314 | GI\|1426069 | GI\|1099415 | GI\|1389303 | GI\|2103515 |
| GI\|1548168 | GI\|2141183 | GI\|666684 | GI\|3173567 | GI\|2111406 |
| GI\|2714531 | GI\|2718954 | GI\|1182737 | GI\|2617817 | GI\|1109119 |
| GI\|3162085 | GI\|2191680 | GI\|2432100 | GI\|2141166 | GI\|3405896 |
| GI\|673514 | GI\|831676 | GI\|2115783 | GI\|891906 | GI\|2969813 |
| GI\|4110658 | GI\|3232370 | GI\|2189568 | GI\|2215333 | GI\|2839089 |
| GI\|2142138 | GI\|2834575 | GI\|1191963 | GI\|2179345 | GI\|3147260 |
| GI\|2525281 | GI\|1068723 | GI\|2185559 | GI\|2958792 | GI\|2183390 |
| GI\|2631015 | GI\|1190314 | GI\|1520983 | GI\|1685895 | GI\|2240463 |
| GI\|3203959 | GI\|3001978 | GI\|1149200 | GI\|1269934 | GI\|2107186 |
| GI\|767048 | GI\|3153990 | GI\|2159071 | GI\|2618146 | GI\|1064170 |
| GI\|1748319 | GI\|3174796 | GI\|2631056 | GI\|985443 | GI\|1679074 |
| GI\|2216635 | GI\|810869 | GI\|1544421 | GI\|2051032 | GI\|2657287 |
| GI\|2184225 | GI\|1472203 | GI\|1692160 | GI\|1687646 | GI\|697470 |
| GI\|1009937 | GI\|2161827 | GI\|1733156 | GI\|1486874 | GI\|2080358 |
| GI\|2835208 | GI\|3094650 | GI\|2214433 | GI\|2218334 | GI\|2432430 |
| GI\|656953 | GI\|865164 | GI\|2178341 | GI\|2432803 | GI\|2538277 |
| GI\|1231416 | GI\|1157469 | GI\|3149107 | GI\|2155679 | GI\|1634222 |
| GI\|1147099 | GI\|2217195 | GI\|2524189 | GI\|3240393 | GI\|3700837 |
| GI\|2167419 | GI\|2945934 | GI\|2524970 | GI\|2138791 | GI\|908122 |
| GI\|3231778 | GI\|1719469 | GI\|1751182 | GI\|2835197 | GI\|2432144 |
| GI\|1212669 | GI\|885481 | GI\|1398641 | GI\|2206902 | GI\|3057539 |
| GI\|1741729 | GI\|1042590 | GI\|2994907 | GI\|2053778 | GI\|890830 |
| GI\|2955793 | GI\|891548 | GI\|1687588 | GI\|2541504 | GI\|1764615 |
| GI\|1697431 | GI\|2179508 | GI\|3181670 | GI\|2183756 | GI\|3230283 |
| GI\|2177373 | GI\|2839059 | GI\|3182593 | GI\|3869944 | GI\|771553 |
| GI\|1211438 | GI\|2954944 | GI\|694526 | GI\|1379503 | GI\|2056264 |
| GI\|2930110 | GI\|2070642 | GI\|1940891 | GI\|3155289 | GI\|3190867 |
| GI\|2177884 | GI\|789775 | GI\|1925386 | GI\|5339519 | GI\|2537954 |
| GI\|2165555 | GI\|3070014 | GI\|2107859 | GI\|675043 | GI\|869870 |
| GI\|816068 | GI\|2178011 | GI\|1271153 | GI\|685981 | GI\|2230309 |
| GI\|1231112 | GI\|2166602 | GI\|1313788 | GI\|2177487 | GI\|873369 |
| GI\|1152351 | GI\|891744 | GI\|717200 | GI\|2189501 | GI\|1471250 |
| GI\|2112377 | GI\|679658 | GI\|1678709 | GI\|1697746 | GI\|2115520 |
| GI\|2111121 | GI\|839785 | GI\|1023769 | GI\|1647035 | GI\|1385053 |
| GI\|2703544 | GI\|1319591 | GI\|1319036 | GI\|1859553 | GI\|2825465 |
| GI\|2229975 | GI\|771572 | GI\|3154368 | GI\|1740540 | GI\|3215096 |
| GI\|883043 | GI\|1502260 | GI\|2207688 | GI\|1436888 | GI\|1426013 |
| GI\|2955433 | GI\|1670627 | GI\|919369 | GI\|673670 | GI\|2718216 |
| GI\|2715060 | GI\|861690 | GI\|1694462 | GI\|3239652 | GI\|3413101 |
| GI\|2141192 | GI\|2703567 | GI\|3802401 | GI\|3190810 | GI\|4148131 |
| GI\|994204 | GI\|1886979 | GI\|835476 | GI\|1016909 | GI\|2968058 |
| GI\|2110926 | GI\|2155757 | GI\|2957943 | GI\|1919792 | GI\|3057621 |
| GI\|1188883 | GI\|1698017 | GI\|1496296 | GI\|680496 | GI\|2630541 |
| GI\|1549730 | GI\|2617430 | GI\|2955292 | GI\|676255 | GI\|2959188 |
| GI\|2106462 | GI\|2051509 | GI\|1558941 | GI\|3048520 | GI\|751040 |
| GI\|1124481 | GI\|2524934 | GI\|4124377 | GI\|1188649 | GI\|2184557 |
| GI\|2209964 | GI\|2100543 | GI\|2569563 | GI\|3058536 | GI\|2204485 |
| GI\|3087302 | GI\|3154742 | GI\|1687643 | GI\|1182504 | GI\|2220971 |
| GI\|3182061 | GI\|1211001 | | | |
| GI\|3116842 | GI\|1615592 | | | |
| GI\|782116 | GI\|1153038 | | | |
| GI\|3231129 | GI\|823208 | | | |
| GI\|2216521 | GI\|2056563 | | | |
| GI\|1785305 | GI\|1740942 | | | |
| GI\|2229773 | GI\|3069635 | | | |
| GI\|1759218 | | | | |
| GI\|821229 | | | | |
| GI\|2155634 | | | | |
| GI\|1426474 | | | | |
| GI\|2214720 | | | | |
| GI\|2216792 | | | | |
| GI\|2240425 | | | | |
| GI\|854768 | | | | |
| GI\|4089551 | | | | |
| GI\|1329704 | | | | |
| GI\|3151403 | | | | |
| GI\|3090156 | | | | |
| GI\|2177338 | | | | |
| GI\|774407 | | | | |
| GI\|2968580 | | | | |
| GI\|1047231 | | | | |
| GI\|1776165 | | | | |
| GI\|1226962 | | | | |
| GI\|2220444 | | | | |
| GI\|4124202 | | | | |
| GI\|2207710 | | | | |
| GI\|5054970 | | | | |
| GI\|1694027 | | | | |
| GI\|1694133 | | | | |
| GI\|1317334 | | | | |
| GI\|3174317 | | | | |
| GI\|2163639 | | | | |
| GI\|1156914 | | | | |
| GI\|2214845 | | | | |
| GI\|1328950 | | | | |
| GI\|2704667 | | | | |
| GI\|3181720 | | | | |
| GI\|2077689 | | | | |
| GI\|1474363 | | | | |
| GI\|2167664 | | | | |
| GI\|4195611 | | | | |
| GI\|1271101 | | | | |
| GI\|1044095 | | | | |
| GI\|2178961 | | | | |
| GI\|1274863 | | | | |
| GI\|954437 | | | | |
| GI\|1063766 | | | | |
| GI\|3096732 | | | | |
| GI\|1026014 | | | | |
| GI\|1212234 | | | | |
| GI\|2713337 | | | | |

TABLE 3C
Nodes 1

| | | | | |
|---|---|---|---|---|
| GI\|2106361 | GI\|4006671 | GI\|2553160 | GI\|2631921 | GI\|1472123 |
| GI\|1298130 | GI\|1404003 | GI\|1922030 | GI\|943035g | GI\|2217285 |
| GI\|3178079 | GI\|1229554 | GI\|2557242 | GI\|3961758 | GI\|3232314 |
| GI\|1382386 | GI\|2553060 | GI\|2656653 | GI\|2557593 | GI\|3173606 |
| GI\|681271g | GI\|1577585 | GI\|2069141 | GI\|1448676 | GI\|2064231 |
| GI\|2969281 | GI\|2059017 | GI\|1241603 | GI\|2834698 | GI\|1751065 |
| GI\|877158g | GI\|1057558 | GI\|848553g | GI\|1383953 | GI\|2968300 |
| GI\|1265737 | GI\|3148404 | GI\|2177973 | GI\|981453g | GI\|838420g |
| GI\|2899640 | GI\|3152269 | GI\|2569897 | GI\|2987201 | GI\|709617g |
| GI\|3959340 | GI\|2631907 | GI\|2079740 | GI\|3154137 | GI\|3001900 |
| GI\|685371g | GI\|1102303 | GI\|1521981 | GI\|2552522 | GI\|1225127 |
| GI\|2140473 | GI\|1056127 | GI\|667189g | GI\|2216791 | GI\|1392194 |
| GI\|2166568 | GI\|1227608 | GI\|2215243 | GI\|2191425 | GI\|2180221 |
| GI\|2969700 | GI\|944711g | GI\|2064295 | GI\|2569306 | GI\|1238679 |
| GI\|884771g | GI\|1212669 | GI\|2219302 | GI\|1547621 | GI\|2141509 |
| GI\|3539498 | GI\|654424g | GI\|898562g | GI\|1269949 | GI\|2216133 |
| GI\|1670891 | GI\|2180437 | GI\|1379214 | GI\|2077417 | |
| GI\|1760440 | GI\|1264955 | GI\|3190456 | GI\|3148748 | |
| GI\|815244g | GI\|690558g | GI\|5339928 | GI\|3151560 | |
| GI\|2458026 | GI\|750595g | GI\|1231416 | GI\|2220485 | |
| GI\|2191830 | GI\|4075718 | GI\|3240739 | GI\|1523377 | |
| GI\|1926106 | GI\|814695g | GI\|824339g | GI\|1390477 | |
| GI\|2657925 | GI\|756035g | GI\|2215228 | GI\|1218283 | |
| GI\|1046649 | GI\|1422713 | GI\|1271799 | GI\|1548284 | |
| GI\|3190819 | GI\|863689g | GI\|675350g | GI\|2963453 | |
| GI\|2215501 | GI\|1779210 | GI\|3155125 | GI\|4113151 | |
| GI\|5439783 | GI\|1470749 | GI\|1920287 | GI\|869327g | |
| GI\|1162368 | GI\|2553411 | GI\|1137549 | GI\|3801103 | |
| GI\|3147004 | GI\|3182593 | GI\|2630554 | GI\|1721784 | |
| GI\|2186050 | GI\|3214861 | GI\|2079775 | GI\|2837490 | |
| GI\|2617972 | GI\|4110658 | GI\|2945739 | GI\|2969182 | |
| GI\|661912g | GI\|4175726 | GI\|2541504 | GI\|3231142 | |
| GI\|676493g | GI\|1400607 | GI\|873946g | GI\|883261g | |
| GI\|1013005 | GI\|3134705 | GI\|2617817 | GI\|1321069 | |
| GI\|727760g | GI\|876628g | GI\|5055283 | GI\|690326g | |
| GI\|1447936 | GI\|2111484 | GI\|1219947 | GI\|1123323 | |
| GI\|2569781 | GI\|3190867 | GI\|1940891 | GI\|900410g | |
| GI\|1188883 | GI\|2557323 | GI\|3092851 | GI\|2100274 | |
| GI\|3172991 | GI\|2618186 | GI\|883043g | GI\|2189627 | |
| GI\|2167780 | GI\|2538171 | GI\|1577716 | GI\|1921238 | |
| GI\|2552715 | GI\|3214705 | GI\|1004992 | GI\|759814g | |
| GI\|1312849 | GI\|2839303 | GI\|4124063 | GI\|2217845 | |
| GI\|4087432 | GI\|880657g | GI\|870604g | GI\|1693580 | |
| GI\|2834870 | GI\|1447862 | GI\|1049931 | GI\|2184225 | |
| GI\|810117g | GI\|1523633 | GI\|2166979 | GI\|2191228 | |
| GI\|2834591 | GI\|2214958 | GI\|1099880 | GI\|3133534 | |
| GI\|2987845 | GI\|2113033 | GI\|2955952 | GI\|746925g | |
| GI\|3049733 | GI\|1741823 | GI\|1329704 | GI\|3179034 | |
| GI\|2069690 | GI\|2835208 | GI\|1167095 | GI\|3177937 | |
| GI\|4006803 | GI\|815357g | GI\|2945715 | GI\|2215669 | |
| GI\|990464g | GI\|2841668 | GI\|2106945 | GI\|1728255 | |
| GI\|1734698 | GI\|958710g | GI\|2432536 | GI\|923067g | |
| GI\|2217431 | GI\|2107116 | GI\|2183553 | GI\|2552630 | |

TABLE 3D
Nodes 2

| | | | | |
|---|---|---|---|---|
| GI\|2159454 | GI\|1502260 | GI\|1486874 | GI\|1697746 | GI\|1925386 |
| GI\|2166979 | GI\|1231416 | GI\|2051032 | GI\|2178341 | GI\|3190839 |
| GI\|1192046 | GI\|789775g | GI\|885481g | GI\|2432144 | GI\|985443g |
| GI\|2525281 | GI\|3154742 | GI\|1685895 | GI\|1224560 | GI\|1779952 |
| GI\|1792569 | GI\|675043g | GI\|2432430 | GI\|839785g | GI\|1426069 |
| GI\|1212669 | GI\|1697431 | GI\|3182593 | GI\|882836g | GI\|1109119 |
| GI\|1548168 | GI\|2955793 | GI\|3182061 | GI\|3000750 | GI\|697470g |
| GI\|1149200 | GI\|2214433 | GI\|1544538 | GI\|1544421 | GI\|986104g |
| GI\|2714531 | GI\|3148463 | GI\|2524934 | GI\|1319036 | GI\|831641g |
| GI\|2713314 | GI\|1472203 | GI\|673670g | GI\|2631056 | GI\|2524970 |
| GI\|4110658 | GI\|1520983 | GI\|2240463 | GI\|666684g | GI\|3057539 |
| GI\|3231778 | GI\|2930110 | GI\|1389303 | GI\|680496g | GI\|1064170 |
| GI\|2432100 | GI\|2834575 | GI\|3230015 | GI\|1436888 | GI\|2541504 |
| GI\|2835208 | GI\|1496296 | GI\|2718954 | GI\|1044095 | GI\|1156914 |
| GI\|2167419 | GI\|2106462 | GI\|1719469 | GI\|959821g | GI\|3070014 |
| GI\|1549730 | GI\|2955433 | GI\|2191680 | GI\|2220971 | GI\|3151403 |
| GI\|2631015 | GI\|2715060 | GI\|2056264 | GI\|4077581 | GI\|2617817 |
| GI\|1687646 | GI\|1271153 | GI\|2106972 | GI\|2618146 | GI\|2214845 |
| GI\|767048g | GI\|2180417 | GI\|1211438 | GI\|3173567 | GI\|2986424 |
| GI\|2155757 | GI\|1319591 | GI\|2100274 | GI\|751040g | GI\|3154160 |
| GI\|1147099 | GI\|1210223 | GI\|892706g | GI\|2657287 | GI\|2183390 |
| GI\|2209964 | GI\|2217431 | GI\|2630541 | GI\|2432235 | GI\|2240940 |
| GI\|869870 | GI\|2185559 | GI\|3069635 | GI\|3214705 | GI\|2838269 |
| GI\|1741729 | GI\|1751182 | GI\|1398641 | GI\|1099415 | GI\|3190867 |
| GI\|2216635 | GI\|717200g | GI\|694526g | GI\|2100543 | GI\|1238840 |
| GI\|1152351 | GI\|1471250 | GI\|3215096 | GI\|2969813 | GI\|2240425 |
| GI\|673514 | GI\|891548g | GI\|1940891 | GI\|2835197 | GI\|2458143 |
| GI\|656953 | GI\|4124210 | GI\|3869944 | GI\|2215460 | GI\|2178011 |
| GI\|2954944 | GI\|2177373 | GI\|2955292 | GI\|774407g | GI\|2185500 |
| GI\|1009937 | GI\|3178750 | GI\|2101007 | GI\|2056809 | GI\|2188816 |
| GI\|2159071 | GI\|2141183 | GI\|2217195 | GI\|3241587 | GI\|2142013 |
| GI\|3162085 | GI\|2538277 | GI\|3058260 | GI\|2969477 | GI\|2107862 |
| GI\|2138791 | GI\|2184225 | GI\|2185067 | GI\|2216521 | GI\|2631622 |
| GI\|1188883 | GI\|1496324 | GI\|2656603 | GI\|2051509 | GI\|1047501 |
| GI\|1191963 | GI\|2155634 | GI\|2569298 | GI\|3181670 | GI\|5054970 |
| GI\|2142138 | GI\|1687643 | GI\|994204g | GI\|2111087 | GI\|1694133 |
| GI\|1124481 | GI\|2657687 | GI\|1269934 | GI\|1764615 | GI\|3181720 |
| GI\|2569563 | GI\|1068723 | GI\|2111121 | GI\|2077711 | GI\|2839059 |
| GI\|1670627 | GI\|891744g | GI\|2703544 | GI\|816068g | GI\|1297931 |
| GI\|831676g | GI\|2703567 | GI\|2718216 | GI\|2957943 | GI\|1186402 |
| GI\|3203959 | GI\|883043g | GI\|1692160 | GI\|3230283 | GI\|1748319 |
| GI\|2229975 | GI\|865164g | GI\|1329704 | GI\|3413101 | GI\|2053778 |
| GI\|1426093 | GI\|2617430 | GI\|771572g | GI\|1124600 | GI\|2103515 |
| GI\|1694462 | GI\|2077802 | GI\|2115783 | GI\|2177487 | GI\|2945645 |
| GI\|2141192 | GI\|2107859 | GI\|3230629 | GI\|2112377 | GI\|3231631 |
| GI\|2155679 | GI\|2218904 | GI\|880662g | GI\|1109117 | GI\|890830g |
| GI\|2524189 | GI\|1231112 | GI\|2945934 | GI\|685981g | GI\|958978g |
| GI\|2112539 | GI\|3190825 | GI\|1118450 | GI\|2070642 | GI\|2206902 |
| GI\|1042590 | GI\|1425855 | GI\|1190314 | GI\|1218594 | GI\|1016909 |
| GI\|3232370 | GI\|2179508 | GI\|1224626 | GI\|3153990 | GI\|2557019 |
| GI\|2166602 | GI\|861690g | GI\|2184570 | GI\|1070800 | GI\|984989g |
| GI\|3405896 | GI\|2215024 | GI\|2994907 | GI\|2189568 | GI\|900124g |
| GI\|2177884 | GI\|883033g | GI\|3149107 | GI\|3240393 | GI\|3094813 |
| GI\|2229773 | GI\|2141241 | GI\|2538171 | | |
| GI\|3239652 | GI\|1203526 | GI\|2189441 | | |
| GI\|4124202 | GI\|2162333 | GI\|1634222 | | |
| GI\|2107017 | GI\|2703845 | GI\|2141904 | | |
| GI\|2111406 | GI\|835476g | GI\|2220543 | | |
| GI\|2210367 | GI\|2955952 | GI\|2216356 | | |
| GI\|1859553 | GI\|2524458 | GI\|3163643 | | |
| GI\|1775317 | GI\|2107186 | GI\|1063766 | | |
| GI\|3094650 | GI\|2207710 | GI\|1886979 | | |
| GI\|2620691 | GI\|1182504 | GI\|815372g | | |
| GI\|2220023 | GI\|1919792 | GI\|657198g | | |
| GI\|875862g | GI\|2836273 | GI\|1271101 | | |
| GI\|1157469 | GI\|2178571 | GI\|659417g | | |
| GI\|1153038 | GI\|2432803 | GI\|679658g | | |
| GI\|1698017 | GI\|766634g | GI\|1496524 | | |
| GI\|2188377 | GI\|2216133 | GI\|2215333 | | |
| GI\|781747g | GI\|2184103 | GI\|2080614 | | |
| GI\|2204485 | GI\|815330g | GI\|1489578 | | |
| GI\|3048520 | GI\|3182434 | GI\|3432904 | | |
| GI\|1615592 | GI\|2716905 | GI\|2057038 | | |
| GI\|1493108 | GI\|854768g | GI\|2064562 | | |
| GI\|2100083 | GI\|1190804 | GI\|3087302 | | |
| GI\|4124063 | GI\|3250604 | GI\|2703053 | | |
| GI\|1733156 | GI\|1118736 | GI\|2618096 | | |
| GI\|900225g | GI\|1647035 | GI\|2167664 | | |
| GI\|1558941 | GI\|874858g | GI\|3202922 | | |
| GI\|1547995 | GI\|664308g | GI\|1379503 | | |
| GI\|3016666 | GI\|1689729 | GI\|2077689 | | |
| GI\|2177973 | GI\|759218g | GI\|1423619 | | |
| GI\|2110926 | GI\|986432g | GI\|1426013 | | |
| GI\|1776165 | GI\|1860336 | GI\|2106439 | | |
| GI\|994995g | GI\|1548308 | GI\|2158953 | | |
| GI\|2834557 | GI\|1229241 | GI\|1751134 | | |
| GI\|2184081 | GI\|1928812 | GI\|2155834 | | |
| GI\|1471634 | GI\|2216792 | GI\|1426474 | | |
| GI\|2191425 | GI\|2620979 | GI\|2220064 | | |
| GI\|1023769 | GI\|1792305 | GI\|4124377 | | |
| GI\|1182737 | GI\|2702889 | | | |
| GI\|2210002 | GI\|1099787 | | | |
| GI\|3230029 | GI\|2179345 | | | |
| GI\|4195611 | GI\|1920153 | | | |
| GI\|3870010 | GI\|919369g | | | |
| GI\|869327g | GI\|2702845 | | | |
| GI\|3190810 | GI\|2556872 | | | |
| GI\|1026014 | GI\|3700837 | | | |
| GI\|2525287 | GI\|901787g | | | |
| GI\|3116842 | GI\|1328950 | | | |
| GI\|1298130 | GI\|757275g | | | |
| GI\|2214720 | GI\|824112g | | | |
| GI\|2179534 | GI\|3181309 | | | |
| GI\|1687588 | GI\|874758g | | | |
| GI\|2945212 | GI\|1124612 | | | |
| GI\|2219395 | GI\|2103505 | | | |

What is claimed is:

1. A method of assessing whether a patient is afflicted with prostate cancer, the method comprising comparing:
a) the level of expression of a marker gene in a patient sample, wherein the marker gene is selected from the group consisting of the marker genes listed in Tables 1-3D, and
b) the normal level of expression of the marker gene in a control non-prostate cancer sample,
wherein a significant increase between the level of expression of the marker gene in the patient sample and the normal level is an indication that the patient is afflicted with prostate cancer.

2. A method for determining whether prostate cancer has metastasized in a patient, the method comprising comparing:
a) the level of expression of one or several prostate cancer marker genes in a patient sample, and
b) the normal level or non-metastatic level of expression of one or several of said marker genes in a control sample
wherein at least one of said marker genes is selected from the group consisting of the marker genes listed in Tables 1-3D, and a significant increase in the level of expression of one or several of said marker genes in the patient sample from the normal level or non-metastatic level is an indication that the prostate cancer has mestastasized.

3. A method for assessing the aggressiveness of prostate cancer comprising comparing:
a) the level of expression of one or several prostate cancer marker genes in a sample, and
b) the normal level of expression of one or several of said marker genes in a control sample,
wherein at least one of said marker genes is selected from the marker genes of Tables 1-3D, and a significant increase in the level of expression of one or several of said marker genes in the sample from the normal level is an indication that the cancer is aggressive.

4. A method for assessing the indolence of prostate cancer comprising comparing:
a) the level of expression of one or several prostate cancer marker genes in a sample, and
b) the normal level of expression of one or several of said marker genes in a control sample,
wherein at least one of said marker genes is selected from the marker genes of Tables 1-3D, and a significant decrease in the level of expression of one or several of said marker genes in the sample from the normal level is an indication that the cancer is indolent.

5. A method for determining whether prostate cancer has metastasized to the liver, or is likely to metastasize to the liver, the method comprising comparing:
a) the level of expression of one or several prostate cancer marker genes in a patient sample, wherein at least one such marker gene is selected from the marker genes of Table 3A and
b) the level of expression of the same marker gene(s) in a sample from a control subject having non-metastasized prostate cancer
wherein a significantly increased level of expression in the patient sample, relative to the level of expression in the control, is an indication that the patient is afflicted with metastatic prostate cancer that has metastasized to the liver, or is likely to metastasize to the liver.

6. A method for determining whether prostate cancer has metastasized to bone tissue, or is likely to metastasize to bone tissue, the method comprising comparing:
a) the level of expression of one or several prostate cancer marker genes in a patient sample, wherein at least one such marker gene is selected from the marker genes of Table 3B and
b) the level of expression of the same marker gene(s) in a sample from a control subject having non-metastasized prostate cancer
wherein a significantly increased level of expression in the patient sample, relative to the level of expression in the control, is an indication that the patient is afflicted with metastatic prostate cancer that has metastasized to bone tissue, or is likely to metastasize to bone tissue.

7. A method for determining whether prostate cancer has metastasized to lymph nodes, or is likely to metastasize to lymph nodes, the method comprising comparing:
a) the level of expression of one or several prostate cancer marker genes in a patient sample, wherein at least one such marker gene is selected from the marker genes of Tables 3C or 3D, and
b) the level of expression of the same marker gene(s) in a sample from a control subject having non-metastasized prostate cancer
wherein a significantly increased level of expression in the patient sample, relative to the level of expression in the control, is an indication that the patient is afflicted with metastatic prostate cancer that has metastasized to lymph nodes, or is likely to metastasize to lymph nodes.

8. The method of claims 1-7, wherein the marker gene corresponds to a secreted protein.

9. The method of claims 1-7, wherein the marker gene corresponds to a transcribed polynucleotide or portion thereof, wherein the polynucleotide comprises the marker gene.

10. The method of claims 1-7, wherein the sample comprises cells obtained from the patient.

11. The method of claim 10, wherein the sample is a prostate tissue sample.

12. The method of claim 10, wherein the cells are in a fluid selected from the group consisting of blood fluids, semen, prostate fluid, lymph and urine.

13. The method of claims 1-7, wherein the level of expression of the marker gene in the sample is assessed by detecting the presence in the sample of a protein or protein fragment corresponding to the marker gene.

14. The method of claim 13, wherein the presence of the protein or protein fragment is detected using a reagent which specifically binds with the protein or protein fragment.

15. The method of claim 14, wherein the reagent is selected from the group consisting of an antibody, an antibody derivative, and an antibody fragment.

16. The method of claims 1-7, wherein the level of expression of the marker gene in the sample is assessed by detecting the presence in the sample of a transcribed polynucleotide or portion thereof, wherein the transcribed polynucleotide comprises the marker gene.

17. The method of claim 16, wherein the transcribed polynucleotide is an mRNA.

18. The method of claim 16, wherein the transcribed polynucleotide is a cDNA.

19. The method of claim 16, wherein the step of detecting further comprises amplifying the transcribed polynucleotide.

20. The method of claims 1-7, wherein the level of expression of the marker gene in the sample is assessed by detecting the presence in the sample of a transcribed polynucleotide which anneals with the marker gene or anneals with a portion of a polynucleotide wherein the polynucleotide comprises the marker gene, under stringent hybridization conditions.

21. A method for monitoring the progression of prostate cancer in a patient, the method comprising:
a) detecting in a patient sample at a first point in time, the expression of a marker gene, wherein the marker gene is selected from the group consisting of the marker genes listed in Tables 1-3D;
b) repeating step a) at a subsequent point in time; and
c) comparing the level of expression detected in steps a) and b), and therefrom monitoring the progression of prostate cancer.

22. The method of claim 21, wherein the marker gene corresponds to a secreted protein.

23. The method of claim 21, wherein the marker gene corresponds to a transcribed polynucleotide or portion thereof, wherein the polynucleotide comprises the marker gene.

24. The method of claim 21, wherein the sample comprises cells obtained from the patient.

25. The method of claim 24, wherein the patient sample is a prostate tissue sample.

26. The method of claim 21, wherein between the first point in time and the subsequent point in time, the patient has undergone surgery to remove prostate tissue.

27. The method of claim 21, wherein between the first point in time and the subsequent point in time, the patient has undergone chemotherapy to remove prostate cancer cells.
